SEQUENCE LISTING (196 sequences)

SEQ ID NO: 1 - RNA, 4319 nt, Severe acute respiratory syndrome coronavirus 2
  source 1..4319: mol_type = genomic RNA
gggaataaactagtattcttctggtccccacagactcagagagaacccgccaccatgttcgtgttcctggtgctgctgcctctggtgtccagccagtgtgtgaacctgaccaccagaacacagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgtttaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccaggacgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggaatctgcgccagctaccagacacagacaaacagccctcggagagccagaagcgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagcaggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccagaatgcccaggcactgaacaccctggtcaagcagctgtcctccaacttcggcgccatcagctctgtgctgaacgatatcctgagcagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatatgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctgaagggcgtgaaactgcactacacatgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcagcggccgcggccgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 2 - DNA, 22 bp, synthetic construct
  source 1..22: mol_type = other DNA
  modified_base 1: mod_base = OTHER; note = Nucleotide with phosphorothioate linkage
  modified_base 2: mod_base = OTHER; note = Nucleotide with phosphorothioate linkage
  modified_base 3: mod_base = OTHER; note = Nucleotide with phosphorothioate linkage
  modified_base 4: mod_base = OTHER; note = Nucleotide with phosphorothioate linkage
  modified_base 5: mod_base = OTHER; note = Nucleotide with phosphorothioate linkage
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = Nucleotide with phosphorothioate linkage
  modified_base 17: mod_base = OTHER; note = Nucleotide with phosphorothioate linkage
  modified_base 18: mod_base = OTHER; note = Nucleotide with phosphorothioate linkage
  modified_base 19: mod_base = OTHER; note = Nucleotide with phosphorothioate linkage
  modified_base 20: mod_base = OTHER; note = Nucleotide with phosphorothioate linkage
  modified_base 21: mod_base = OTHER; note = Nucleotide with phosphorothioate linkage
tgactgtgaacgttcgagatga

SEQ ID NO: 3 - DNA, 24 bp, synthetic construct
  source 1..24: mol_type = other DNA
  modified_base 1: mod_base = OTHER; note = Nucleotide with phosphorothioate linkage
  modified_base 2: mod_base = OTHER; note = Nucleotide with phosphorothioate linkage
  modified_base 3: mod_base = OTHER; note = Nucleotide with phosphorothioate linkage
  modified_base 4: mod_base = OTHER; note = Nucleotide with phosphorothioate linkage
  modified_base 5: mod_base = OTHER; note = Nucleotide with phosphorothioate linkage
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = Nucleotide with phosphorothioate linkage
  modified_base 17: mod_base = OTHER; note = Nucleotide with phosphorothioate linkage
  modified_base 18: mod_base = OTHER; note = Nucleotide with phosphorothioate linkage
  modified_base 19: mod_base = OTHER; note = Nucleotide with phosphorothioate linkage
  modified_base 20: mod_base = OTHER; note = Nucleotide with phosphorothioate linkage
  modified_base 21: mod_base = OTHER; note = Nucleotide with phosphorothioate linkage
  modified_base 22: mod_base = OTHER; note = Nucleotide with phosphorothioate linkage
  modified_base 23: mod_base = OTHER; note = Nucleotide with phosphorothioate linkage
tcgtcgttttgtcgttttgtcgtt

SEQ ID NO: 4 - DNA, 20 bp, synthetic construct
  source 1..20: mol_type = other DNA
  modified_base 1: mod_base = OTHER; note = Nucleotide with phosphorothioate linkage
  modified_base 2: mod_base = OTHER; note = Nucleotide with phosphorothioate linkage
  modified_base 15: mod_base = OTHER; note = Nucleotide with phosphorothioate linkage
  modified_base 16: mod_base = OTHER; note = Nucleotide with phosphorothioate linkage
  modified_base 17: mod_base = OTHER; note = Nucleotide with phosphorothioate linkage
  modified_base 18: mod_base = OTHER; note = Nucleotide with phosphorothioate linkage
  modified_base 19: mod_base = OTHER; note = Nucleotide with phosphorothioate linkage
ggggtcaacgttgagggggg

SEQ ID NO: 5 - DNA, 20 bp, synthetic construct
  source 1..20: mol_type = other DNA
  modified_base 1: mod_base = OTHER; note = Nucleotide with phosphorothioate linkage
  modified_base 2: mod_base = OTHER; note = Nucleotide with phosphorothioate linkage
  modified_base 3: mod_base = OTHER; note = Nucleotide with phosphorothioate linkage
  modified_base 4: mod_base = OTHER; note = Nucleotide with phosphorothioate linkage
  modified_base 5: mod_base = OTHER; note = Nucleotide with phosphorothioate linkage
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = Nucleotide with phosphorothioate linkage
  modified_base 17: mod_base = OTHER; note = Nucleotide with phosphorothioate linkage
  modified_base 18: mod_base = OTHER; note = Nucleotide with phosphorothioate linkage
  modified_base 19: mod_base = OTHER; note = Nucleotide with phosphorothioate linkage
tccatgacgttcctgatgct

SEQ ID NO: 6 - DNA, 20 bp, synthetic construct
  source 1..20: mol_type = other DNA
  modified_base 1: mod_base = OTHER; note = Nucleotide with phosphorothioate linkage
  modified_base 2: mod_base = OTHER; note = Nucleotide with phosphorothioate linkage
  modified_base 15: mod_base = OTHER; note = Nucleotide with phosphorothioate linkage
  modified_base 16: mod_base = OTHER; note = Nucleotide with phosphorothioate linkage
  modified_base 17: mod_base = OTHER; note = Nucleotide with phosphorothioate linkage
  modified_base 18: mod_base = OTHER; note = Nucleotide with phosphorothioate linkage
  modified_base 19: mod_base = OTHER; note = Nucleotide with phosphorothioate linkage
gggggacgatcgtcgggggg

SEQ ID NO: 7 - DNA, 21 bp, synthetic construct
  source 1..21: mol_type = other DNA
  modified_base 1: mod_base = OTHER; note = Nucleotide with phosphorothioate linkage
  modified_base 2: mod_base = OTHER; note = Nucleotide with phosphorothioate linkage
  modified_base 3: mod_base = OTHER; note = Nucleotide with phosphorothioate linkage
  modified_base 16: mod_base = OTHER; note = Nucleotide with phosphorothioate linkage
  modified_base 17: mod_base = OTHER; note = Nucleotide with phosphorothioate linkage
  modified_base 18: mod_base = OTHER; note = Nucleotide with phosphorothioate linkage
  modified_base 19: mod_base = OTHER; note = Nucleotide with phosphorothioate linkage
  modified_base 20: mod_base = OTHER; note = Nucleotide with phosphorothioate linkage
ggggacgacgtcgtggggggg

SEQ ID NO: 8 - DNA, 22 bp, synthetic construct
  source 1..22: mol_type = other DNA
  modified_base 1: mod_base = OTHER; note = Nucleotide with phosphorothioate linkage
  modified_base 2: mod_base = OTHER; note = Nucleotide with phosphorothioate linkage
  modified_base 3: mod_base = OTHER; note = Nucleotide with phosphorothioate linkage
  modified_base 4: mod_base = OTHER; note = Nucleotide with phosphorothioate linkage
  modified_base 5: mod_base = OTHER; note = Nucleotide with phosphorothioate linkage
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = Nucleotide with phosphorothioate linkage
  modified_base 17: mod_base = OTHER; note = Nucleotide with phosphorothioate linkage
  modified_base 18: mod_base = OTHER; note = Nucleotide with phosphorothioate linkage
  modified_base 19: mod_base = OTHER; note = Nucleotide with phosphorothioate linkage
  modified_base 20: mod_base = OTHER; note = Nucleotide with phosphorothioate linkage
  modified_base 21: mod_base = OTHER; note = Nucleotide with phosphorothioate linkage
tcgtcgttttcggcgcgcgccg

SEQ ID NO: 9 - DNA, 21 bp, synthetic construct
  source 1..21: mol_type = other DNA
  modified_base 1: mod_base = OTHER; note = Nucleotide with phosphorothioate linkage
  modified_base 2: mod_base = OTHER; note = Nucleotide with phosphorothioate linkage
  modified_base 3: mod_base = OTHER; note = Nucleotide with phosphorothioate linkage
  modified_base 4: mod_base = OTHER; note = Nucleotide with phosphorothioate linkage
  modified_base 5: mod_base = OTHER; note = Nucleotide with phosphorothioate linkage
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = Nucleotide with phosphorothioate linkage
  modified_base 17: mod_base = OTHER; note = Nucleotide with phosphorothioate linkage
  modified_base 18: mod_base = OTHER; note = Nucleotide with phosphorothioate linkage
  modified_base 19: mod_base = OTHER; note = Nucleotide with phosphorothioate linkage
  modified_base 20: mod_base = OTHER; note = Nucleotide with phosphorothioate linkage
tcgtcgtttttcggtcgtttt

SEQ ID NO: 10 - DNA, 185 bp, synthetic construct
  source 1..185: mol_type = other DNA
  misc_difference 1: note = a, c, t, g, unknown or other
  misc_difference 2: note = a, c, t, g, unknown or other
  misc_difference 3: note = a, c, t, g, unknown or other
  misc_difference 7: note = a, c, t, g, unknown or other
  misc_difference 8: note = a, c, t, g, unknown or other
  misc_difference 12: note = a, c, t, g, unknown or other
  misc_difference 13: note = a, c, t, g, unknown or other
  misc_difference 17: note = a, c, t, g, unknown or other
  misc_difference 18: note = a, c, t, g, unknown or other
  misc_difference 22: note = a, c, t, g, unknown or other
  misc_difference 23: note = a, c, t, g, unknown or other
  misc_difference 24: note = a, c, t, g, unknown or other
  misc_difference 25: note = a, c, t, g, unknown or other
  misc_difference 26: note = a, c, t, g, unknown or other
  misc_difference 29: note = a, c, t, g, unknown or other
  misc_difference 30: note = a, c, t, g, unknown or other
  misc_difference 33: note = a, c, t, g, unknown or other
  misc_difference 34: note = a, c, t, g, unknown or other
  misc_difference 37: note = a, c, t, g, unknown or other
  misc_difference 38: note = a, c, t, g, unknown or other
  misc_difference 41: note = a, c, t, g, unknown or other
  misc_difference 42: note = a, c, t, g, unknown or other
  misc_difference 45: note = a, c, t, g, unknown or other
  misc_difference 46: note = a, c, t, g, unknown or other
  misc_difference 49: note = a, c, t, g, unknown or other
  misc_difference 50: note = a, c, t, g, unknown or other
  misc_difference 53: note = a, c, t, g, unknown or other
  misc_difference 54: note = a, c, t, g, unknown or other
  misc_difference 57: note = a, c, t, g, unknown or other
  misc_difference 58: note = a, c, t, g, unknown or other
  misc_difference 61: note = a, c, t, g, unknown or other
  misc_difference 62: note = a, c, t, g, unknown or other
  misc_difference 65: note = a, c, t, g, unknown or other
  misc_difference 66: note = a, c, t, g, unknown or other
  misc_difference 69: note = a, c, t, g, unknown or other
  misc_difference 70: note = a, c, t, g, unknown or other
  misc_difference 73: note = a, c, t, g, unknown or other
  misc_difference 74: note = a, c, t, g, unknown or other
  misc_difference 77: note = a, c, t, g, unknown or other
  misc_difference 78: note = a, c, t, g, unknown or other
  misc_difference 81: note = a, c, t, g, unknown or other
  misc_difference 82: note = a, c, t, g, unknown or other
  misc_difference 85: note = a, c, t, g, unknown or other
  misc_difference 86: note = a, c, t, g, unknown or other
  misc_difference 89: note = a, c, t, g, unknown or other
  misc_difference 90: note = a, c, t, g, unknown or other
  misc_difference 93: note = a, c, t, g, unknown or other
  misc_difference 94: note = a, c, t, g, unknown or other
  misc_difference 97: note = a, c, t, g, unknown or other
  misc_difference 98: note = a, c, t, g, unknown or other
  misc_difference 101: note = a, c, t, g, unknown or other
  misc_difference 102: note = a, c, t, g, unknown or other
  misc_difference 105: note = a, c, t, g, unknown or other
  misc_difference 106: note = a, c, t, g, unknown or other
  misc_difference 109: note = a, c, t, g, unknown or other
  misc_difference 110: note = a, c, t, g, unknown or other
  misc_difference 113: note = a, c, t, g, unknown or other
  misc_difference 114: note = a, c, t, g, unknown or other
  misc_difference 117: note = a, c, t, g, unknown or other
  misc_difference 118: note = a, c, t, g, unknown or other
  misc_difference 121: note = a, c, t, g, unknown or other
  misc_difference 122: note = a, c, t, g, unknown or other
  misc_difference 125: note = a, c, t, g, unknown or other
  misc_difference 126: note = a, c, t, g, unknown or other
  misc_difference 129: note = a, c, t, g, unknown or other
  misc_difference 130: note = a, c, t, g, unknown or other
  misc_difference 133: note = a, c, t, g, unknown or other
  misc_difference 134: note = a, c, t, g, unknown or other
  misc_difference 137: note = a, c, t, g, unknown or other
  misc_difference 138: note = a, c, t, g, unknown or other
  misc_difference 141: note = a, c, t, g, unknown or other
  misc_difference 142: note = a, c, t, g, unknown or other
  misc_difference 145: note = a, c, t, g, unknown or other
  misc_difference 146: note = a, c, t, g, unknown or other
  misc_difference 149: note = a, c, t, g, unknown or other
  misc_difference 150: note = a, c, t, g, unknown or other
  misc_difference 153: note = a, c, t, g, unknown or other
  misc_difference 154: note = a, c, t, g, unknown or other
  misc_difference 157: note = a, c, t, g, unknown or other
  misc_difference 158: note = a, c, t, g, unknown or other
  misc_difference 161: note = a, c, t, g, unknown or other
  misc_difference 162: note = a, c, t, g, unknown or other
  misc_difference 165: note = a, c, t, g, unknown or other
  misc_difference 166: note = a, c, t, g, unknown or other
  misc_difference 169: note = a, c, t, g, unknown or other
  misc_difference 170: note = a, c, t, g, unknown or other
  misc_difference 173: note = a, c, t, g, unknown or other
  misc_difference 174: note = a, c, t, g, unknown or other
  misc_difference 177: note = a, c, t, g, unknown or other
  misc_difference 178: note = a, c, t, g, unknown or other
  misc_difference 181: note = a, c, t, g, unknown or other
  misc_difference 182: note = a, c, t, g, unknown or other
  misc_difference 1..3: note = This region may encompass 0-3 nucleotides
  misc_difference 4..23: note = This region may encompass 1-4 tcg(n)q repeating units, wherein q = 0-2
  misc_difference 24..25: note = This region may encompass 0-2 nucleotides
  misc_difference 26..185: note = This region may encompass 1-20 nncgnn(cg)p repeating units, wherein p = 0-1
nnntcgnntcgnntcgnntcgnnnnnncgnncgnncgnncgnncgnncgnncgnncgnncgnncgnncgnncgnncgnncgnncgnncgnncgnncgnncgnncgnncgnncgnncgnncgnncgnncgnncgnncgnncgnncgnncgnncgnncgnncgnncgnncgnncgnncgnncgnncg

SEQ ID NO: 11 - RNA, 18 nt, synthetic construct
  source 1..18: mol_type = other RNA
ccgtctgttgtgtgactc

SEQ ID NO: 12-14 - skipped (3 entries)

SEQ ID NO: 15 - RNA, 20 nt, synthetic construct
  source 1..20: mol_type = other RNA
gccaccgagcgttgtgtacc

SEQ ID NO: 16 - RNA, 20 nt, synthetic construct
  source 1..20: mol_type = other RNA
gccaccgagcattgtgaacc

SEQ ID NO: 17 - RNA, 20 nt, synthetic construct
  source 1..20: mol_type = other RNA
gccaccgagcattgagaacc

SEQ ID NO: 18 - RNA, 20 nt, synthetic construct
  source 1..20: mol_type = other RNA
gccaccgagcaatgagaacc

SEQ ID NO: 19 - RNA, 20 nt, synthetic construct
  source 1..20: mol_type = other RNA
gccaccgagcaaggtgaacc

SEQ ID NO: 20 - RNA, 20 nt, synthetic construct
  source 1..20: mol_type = other RNA
tatatatatatatatatata

SEQ ID NO: 21 - RNA, 20 nt, synthetic construct
  source 1..20: mol_type = other RNA
ttattattattattattatt

SEQ ID NO: 22 - RNA, 20 nt, synthetic construct
  source 1..20: mol_type = other RNA
tttatttatttatttattta

SEQ ID NO: 23 - RNA, 20 nt, synthetic construct
  source 1..20: mol_type = other RNA
ttttattttattttatttta

SEQ ID NO: 24 - RNA, 20 nt, synthetic construct
  source 1..20: mol_type = other RNA
tgtgtgtgtgtgtgtgtgtg

SEQ ID NO: 25 - RNA, 20 nt, synthetic construct
  source 1..20: mol_type = other RNA
ttgttgttgttgttgttgtt

SEQ ID NO: 26 - RNA, 20 nt, synthetic construct
  source 1..20: mol_type = other RNA
tttgtttgtttgtttgtttg

SEQ ID NO: 27 - RNA, 20 nt, synthetic construct
  source 1..20: mol_type = other RNA
ttttgttttgttttgttttg

SEQ ID NO: 28 - RNA, 16 nt, synthetic construct
  source 1..16: mol_type = other RNA
ccgagccgcttgtccc

SEQ ID NO: 29 - RNA, 16 nt, synthetic construct
  source 1..16: mol_type = other RNA
ccgagccgctttcccc

SEQ ID NO: 30 - DNA, 16 bp, synthetic construct
  source 1..16: mol_type = other DNA
  misc_feature 1..10: note = RNA
  misc_feature 11: note = DNA
  misc_feature 12: note = RNA
  misc_feature 13: note = DNA
  misc_feature 14..16: note = RNA
ccgagccgcttttccc

SEQ ID NO: 31 - RNA, 16 nt, synthetic construct
  source 1..16: mol_type = other RNA
ccgagccgctgttccc

SEQ ID NO: 32 - RNA, 16 nt, synthetic construct
  source 1..16: mol_type = other RNA
ccgagccgcctgtccc

SEQ ID NO: 33 - RNA, 16 nt, synthetic construct
  source 1..16: mol_type = other RNA
ccgagccgctattccc

SEQ ID NO: 34 - DNA, 16 bp, synthetic construct
  source 1..16: mol_type = other DNA
  misc_feature 1..12: note = RNA
  misc_feature 13: note = DNA
  misc_feature 14..16: note = RNA
ccgagccgcttttccc

SEQ ID NO: 35 - DNA, 16 bp, synthetic construct
  source 1..16: mol_type = other DNA
  misc_feature 1..9: note = RNA
  misc_feature 10: note = DNA
  misc_feature 11..16: note = RNA
ccgagccgcttgtccc

SEQ ID NO: 36 - RNA, 16 nt, synthetic construct
  source 1..16: mol_type = other RNA
ccgagccgcgtttccc

SEQ ID NO: 37 - RNA, 16 nt, synthetic construct
  source 1..16: mol_type = other RNA
ccgagccgcatttccc

SEQ ID NO: 38 - RNA, 16 nt, synthetic construct
  source 1..16: mol_type = other RNA
ccgagccgcatgtccc

SEQ ID NO: 39 - RNA, 16 nt, synthetic construct
  source 1..16: mol_type = other RNA
ccgagccgcctttccc

SEQ ID NO: 40 - RNA, 16 nt, synthetic construct
  source 1..16: mol_type = other RNA
ccgagccgctttaccc

SEQ ID NO: 41 - RNA, 16 nt, synthetic construct
  source 1..16: mol_type = other RNA
ccgagccgcttgcccc

SEQ ID NO: 42 - RNA, 16 nt, synthetic construct
  source 1..16: mol_type = other RNA
ccgagccgctcttccc

SEQ ID NO: 43 - DNA, 16 bp, synthetic construct
  source 1..16: mol_type = other DNA
  misc_feature 1..9: note = RNA
  misc_feature 10: note = DNA
  misc_feature 11..16: note = RNA
ccgagccgctttcccc

SEQ ID NO: 44 - RNA, 16 nt, synthetic construct
  source 1..16: mol_type = other RNA
ccgagccgcattcccc

SEQ ID NO: 45 - DNA, 16 bp, synthetic construct
  source 1..16: mol_type = other DNA
  misc_feature 1..12: note = RNA
  misc_feature 13: note = DNA
  misc_feature 14..16: note = RNA
ccgagccgcttctccc

SEQ ID NO: 46 - RNA, 16 nt, synthetic construct
  source 1..16: mol_type = other RNA
ccgagccgctctcccc

SEQ ID NO: 47 - DNA, 16 bp, synthetic construct
  source 1..16: mol_type = other DNA
  misc_feature 1..9: note = RNA
  misc_feature 10: note = DNA
  misc_feature 11..16: note = RNA
ccgagccgcttttccc

SEQ ID NO: 48 - RNA, 16 nt, synthetic construct
  source 1..16: mol_type = other RNA
ccgagccgcttgaccc

SEQ ID NO: 49 - RNA, 16 nt, synthetic construct
  source 1..16: mol_type = other RNA
ccgagccgctataccc

SEQ ID NO: 50 - RNA, 16 nt, synthetic construct
  source 1..16: mol_type = other RNA
ccgagccgcgttcccc

SEQ ID NO: 51 - RNA, 16 nt, synthetic construct
  source 1..16: mol_type = other RNA
ccgagccgcatatccc

SEQ ID NO: 52 - RNA, 16 nt, synthetic construct
  source 1..16: mol_type = other RNA
ccgagccgcatacccc

SEQ ID NO: 53 - RNA, 16 nt, synthetic construct
  source 1..16: mol_type = other RNA
ccgagccgctatcccc

SEQ ID NO: 54 - RNA, 16 nt, synthetic construct
  source 1..16: mol_type = other RNA
ccgagccgcgctcccc

SEQ ID NO: 55 - RNA, 16 nt, synthetic construct
  source 1..16: mol_type = other RNA
ccgagccgcctacccc

SEQ ID NO: 56 - RNA, 16 nt, synthetic construct
  source 1..16: mol_type = other RNA
ccgagccgcgtacccc

SEQ ID NO: 57 - RNA, 16 nt, synthetic construct
  source 1..16: mol_type = other RNA
ccgagccgcgtgcccc

SEQ ID NO: 58 - RNA, 16 nt, synthetic construct
  source 1..16: mol_type = other RNA
ccgagccgcctgcccc

SEQ ID NO: 59 - DNA, 16 bp, synthetic construct
  source 1..16: mol_type = other DNA
  misc_feature 1..11: note = RNA
  misc_feature 12: note = DNA
  misc_feature 13..16: note = RNA
ccgagccgcttttccc

SEQ ID NO: 60 - RNA, 16 nt, synthetic construct
  source 1..16: mol_type = other RNA
ccgagccgcttctccc

SEQ ID NO: 61 - RNA, 16 nt, synthetic construct
  source 1..16: mol_type = other RNA
ccgagccgcataaccc

SEQ ID NO: 62 - RNA, 16 nt, synthetic construct
  source 1..16: mol_type = other RNA
ccgagccgcctaaccc

SEQ ID NO: 63 - RNA, 16 nt, synthetic construct
  source 1..16: mol_type = other RNA
ccgagccgcttttccc

SEQ ID NO: 64 - RNA, 16 nt, synthetic construct
  source 1..16: mol_type = other RNA
ccgagccgcattaccc

SEQ ID NO: 65 - RNA, 16 nt, synthetic construct
  source 1..16: mol_type = other RNA
  modified_base 12: mod_base = OTHER; note = Inosine
ccgagccgcttntccc

SEQ ID NO: 66 - RNA, 16 nt, synthetic construct
  source 1..16: mol_type = other RNA
ccgagccgcttatccc

SEQ ID NO: 67 - RNA, 16 nt, synthetic construct
  source 1..16: mol_type = other RNA
ccgagccgcctccccc

SEQ ID NO: 68 - RNA, 16 nt, synthetic construct
  source 1..16: mol_type = other RNA
ccgagccgcatccccc

SEQ ID NO: 69 - RNA, 16 nt, synthetic construct
  source 1..16: mol_type = other RNA
ccgagccgcgtccccc

SEQ ID NO: 70 - RNA, 16 nt, synthetic construct
  source 1..16: mol_type = other RNA
ccgagccgattgtacc

SEQ ID NO: 71 - RNA, 16 nt, synthetic construct
  source 1..16: mol_type = other RNA
ccgagccgatttcacc

SEQ ID NO: 72 - DNA, 16 bp, synthetic construct
  source 1..16: mol_type = other DNA
  misc_feature 1..10: note = RNA
  misc_feature 11: note = DNA
  misc_feature 12: note = RNA
  misc_feature 13: note = DNA
  misc_feature 14..16: note = RNA
ccgagccgattttacc

SEQ ID NO: 73 - RNA, 16 nt, synthetic construct
  source 1..16: mol_type = other RNA
ccgagccgatgttacc

SEQ ID NO: 74 - RNA, 16 nt, synthetic construct
  source 1..16: mol_type = other RNA
ccgagccgactgtacc

SEQ ID NO: 75 - RNA, 16 nt, synthetic construct
  source 1..16: mol_type = other RNA
ccgagccgatattacc

SEQ ID NO: 76 - DNA, 16 bp, synthetic construct
  source 1..16: mol_type = other DNA
  misc_feature 1..12: note = RNA
  misc_feature 13: note = DNA
  misc_feature 14..16: note = RNA
ccgagccgattttacc

SEQ ID NO: 77 - DNA, 16 bp, synthetic construct
  source 1..16: mol_type = other DNA
  misc_feature 1..9: note = RNA
  misc_feature 10: note = DNA
  misc_feature 11..16: note = RNA
ccgagccgattgtacc

SEQ ID NO: 78 - RNA, 16 nt, synthetic construct
  source 1..16: mol_type = other RNA
ccgagccgagtttacc

SEQ ID NO: 79 - RNA, 16 nt, synthetic construct
  source 1..16: mol_type = other RNA
ccgagccgaatttacc

SEQ ID NO: 80 - RNA, 16 nt, synthetic construct
  source 1..16: mol_type = other RNA
ccgagccgaatgtacc

SEQ ID NO: 81 - RNA, 16 nt, synthetic construct
  source 1..16: mol_type = other RNA
ccgagccgactttacc

SEQ ID NO: 82 - RNA, 16 nt, synthetic construct
  source 1..16: mol_type = other RNA
ccgagccgatttaacc

SEQ ID NO: 83 - RNA, 16 nt, synthetic construct
  source 1..16: mol_type = other RNA
ccgagccgattgcacc

SEQ ID NO: 84 - RNA, 16 nt, synthetic construct
  source 1..16: mol_type = other RNA
ccgagccgatcttacc

SEQ ID NO: 85 - DNA, 16 bp, synthetic construct
  source 1..16: mol_type = other DNA
  misc_feature 1..9: note = RNA
  misc_feature 10: note = DNA
  misc_feature 11..16: note = RNA
ccgagccgatttcacc

SEQ ID NO: 86 - RNA, 16 nt, synthetic construct
  source 1..16: mol_type = other RNA
ccgagccgaattcacc

SEQ ID NO: 87 - DNA, 16 bp, synthetic construct
  source 1..16: mol_type = other DNA
  misc_feature 1..12: note = RNA
  misc_feature 13: note = DNA
  misc_feature 14..16: note = RNA
ccgagccgattctacc

SEQ ID NO: 88 - RNA, 16 nt, synthetic construct
  source 1..16: mol_type = other RNA
ccgagccgatctcacc

SEQ ID NO: 89 - DNA, 16 bp, synthetic construct
  source 1..16: mol_type = other DNA
  misc_feature 1..9: note = RNA
  misc_feature 10: note = DNA
  misc_feature 11..16: note = RNA
ccgagccgattttacc

SEQ ID NO: 90 - RNA, 16 nt, synthetic construct
  source 1..16: mol_type = other RNA
ccgagccgattgaacc

SEQ ID NO: 91 - RNA, 16 nt, synthetic construct
  source 1..16: mol_type = other RNA
ccgagccgatataacc

SEQ ID NO: 92 - RNA, 16 nt, synthetic construct
  source 1..16: mol_type = other RNA
ccgagccgagttcacc

SEQ ID NO: 93 - RNA, 16 nt, synthetic construct
  source 1..16: mol_type = other RNA
ccgagccgaatatacc

SEQ ID NO: 94 - RNA, 16 nt, synthetic construct
  source 1..16: mol_type = other RNA
ccgagccgaatacacc

SEQ ID NO: 95 - RNA, 16 nt, synthetic construct
  source 1..16: mol_type = other RNA
ccgagccgatatcacc

SEQ ID NO: 96 - RNA, 16 nt, synthetic construct
  source 1..16: mol_type = other RNA
ccgagccgagctcacc

SEQ ID NO: 97 - RNA, 16 nt, synthetic construct
  source 1..16: mol_type = other RNA
ccgagccgactacacc

SEQ ID NO: 98 - RNA, 16 nt, synthetic construct
  source 1..16: mol_type = other RNA
ccgagccgagtacacc

SEQ ID NO: 99 - RNA, 16 nt, synthetic construct
  source 1..16: mol_type = other RNA
ccgagccgagtgcacc

SEQ ID NO: 100 - RNA, 16 nt, synthetic construct
  source 1..16: mol_type = other RNA
ccgagccgactgcacc

SEQ ID NO: 101 - DNA, 16 bp, synthetic construct
  source 1..16: mol_type = other DNA
  misc_feature 1..11: note = RNA
  misc_feature 12: note = DNA
  misc_feature 13..16: note = RNA
ccgagccgattttacc

SEQ ID NO: 102 - RNA, 16 nt, synthetic construct
  source 1..16: mol_type = other RNA
ccgagccgattctacc

SEQ ID NO: 103 - RNA, 16 nt, synthetic construct
  source 1..16: mol_type = other RNA
ccgagccgaataaacc

SEQ ID NO: 104 - RNA, 16 nt, synthetic construct
  source 1..16: mol_type = other RNA
ccgagccgactaaacc

SEQ ID NO: 105 - RNA, 16 nt, synthetic construct
  source 1..16: mol_type = other RNA
ccgagccgattttacc

SEQ ID NO: 106 - RNA, 16 nt, synthetic construct
  source 1..16: mol_type = other RNA
ccgagccgaattaacc

SEQ ID NO: 107 - RNA, 16 nt, synthetic construct
  source 1..16: mol_type = other RNA
  modified_base 12: mod_base = OTHER; note = Inosine
ccgagccgattntacc

SEQ ID NO: 108 - RNA, 16 nt, synthetic construct
  source 1..16: mol_type = other RNA
ccgagccgattatacc

SEQ ID NO: 109 - RNA, 16 nt, synthetic construct
  source 1..16: mol_type = other RNA
ccgagccgactccacc

SEQ ID NO: 110 - RNA, 16 nt, synthetic construct
  source 1..16: mol_type = other RNA
ccgagccgaatccacc

SEQ ID NO: 111 - RNA, 16 nt, synthetic construct
  source 1..16: mol_type = other RNA
ccgagccgagtccacc

SEQ ID NO: 112 - RNA, 20 nt, synthetic construct
  source 1..20: mol_type = other RNA
tgtccttcaatgtccttcaa

SEQ ID NO: 113 - RNA, 19 nt, synthetic construct
  source 1..19: mol_type = other RNA
tggtaattgaaggacaggt

SEQ ID NO: 114 - RNA, 19 nt, synthetic construct
  source 1..19: mol_type = other RNA
agcttaacctgtccttcaa

SEQ ID NO: 115 - RNA, 18 nt, synthetic construct
  source 1..18: mol_type = other RNA
gttgtggttgtggttgtg

SEQ ID NO: 116 - RNA, 10 nt, synthetic construct
  source 1..10: mol_type = other RNA
gtagtgtgtg

SEQ ID NO: 117 - RNA, 12 nt, synthetic construct
  source 1..12: mol_type = other RNA
gtctgttgtgtg

SEQ ID NO: 118 - RNA, 125 nt, synthetic construct
  source 1..125: mol_type = other RNA
taatacgactcactatagggttggttggtttttttttgggtttttttttttgttttttttttttttttttttttttttttttttttgtggtttttttttggtgtttttttggttttgtttggttt

SEQ ID NO: 119 - RNA, 122 nt, synthetic construct
  source 1..122: mol_type = other RNA
taatacgactcactataggccatcctgtttttttccctttttttttttcttttttttttttttttttttttttttttttttttctcctttttttttcctctttttttccttttctttccttt

SEQ ID NO: 120 - RNA, 122 nt, synthetic construct
  source 1..122: mol_type = other RNA
taatacgactcactatagggaaggaaggaaaaaaaaagggaaaaaaaaaagaaaaaaaaaaaaaaaaaaaaaaaaaaaaaaaagaggaaaaaaaaaggagaaaaaaaggaaaagaaaggaaa

SEQ ID NO: 121 - RNA, 67 nt, synthetic construct
  source 1..67: mol_type = other RNA
taatacgactcactataggccatcctgtttttttccctttttttttttctttttttttttttttttt

SEQ ID NO: 122 - RNA, 42 nt, synthetic construct
  source 1..42: mol_type = other RNA
taatacgactcactataggccatcctgtttttttcccttttt

SEQ ID NO: 123 - RNA, 125 nt, synthetic construct
  source 1..125: mol_type = other RNA
taatacgactcactatagggaaaggaaagaaaaggaaaaaaagaggaaaaaaaaaggagaaaaaaaaaaaaaaaaaaaaaaaaaaaaaaaaaagaaaaaaaaaaagggaaaaaaacaggatggcc

SEQ ID NO: 124 - RNA, 42 nt, synthetic construct
  source 1..42: mol_type = other RNA
taatacgactcactatagggacttgatctgcagagaggccag

SEQ ID NO: 125 - RNA, 41 nt, synthetic construct
  source 1..41: mol_type = other RNA
taatacgactcactatagggccagcccccgattgggggcga

SEQ ID NO: 126 - RNA, 117 nt, synthetic construct
  source 1..117: mol_type = other RNA
taatacgactcactatgggaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 127 - RNA, 24 nt, synthetic construct
  source 1..24: mol_type = other RNA
gacgctgaccctgaagttcatctt

SEQ ID NO: 128 - RNA, 24 nt, synthetic construct
  source 1..24: mol_type = other RNA
aagatgaacttcagggtcagcgtc

SEQ ID NO: 129 - RNA, 23 nt, synthetic construct
  source 1..23: mol_type = other RNA
agatgaacttcagggtcagcgtc

SEQ ID NO: 130 - RNA, 24 nt, synthetic construct
  source 1..24: mol_type = other RNA
aacacacacacacacacacacttt

SEQ ID NO: 131 - RNA, 21 nt, synthetic construct
  source 1..21: mol_type = other RNA
gtgtgtgtgtgtgtgtgtgtt

SEQ ID NO: 132 - RNA, 22 nt, synthetic construct
  source 1..22: mol_type = other RNA
agtgtgtgtgtgtgtgtgtgtt

SEQ ID NO: 133 - RNA, 23 nt, synthetic construct
  source 1..23: mol_type = other RNA
aagtgtgtgtgtgtgtgtgtgtt

SEQ ID NO: 134 - RNA, 24 nt, synthetic construct
  source 1..24: mol_type = other RNA
aaagtgtgtgtgtgtgtgtgtgtt

SEQ ID NO: 135 - RNA, 25 nt, synthetic construct
  source 1..25: mol_type = other RNA
aaaagtgtgtgtgtgtgtgtgtgtt

SEQ ID NO: 136 - RNA, 26 nt, synthetic construct
  source 1..26: mol_type = other RNA
aaaaagtgtgtgtgtgtgtgtgtgtt

SEQ ID NO: 137 - RNA, 24 nt, synthetic construct
  source 1..24: mol_type = other RNA
gacacacacacacacacacacttt

SEQ ID NO: 138 - RNA, 21 nt, synthetic construct
  source 1..21: mol_type = other RNA
gtgtgtgtgtgtgtgtgtgtc

SEQ ID NO: 139 - RNA, 22 nt, synthetic construct
  source 1..22: mol_type = other RNA
agtgtgtgtgtgtgtgtgtgtc

SEQ ID NO: 140 - RNA, 23 nt, synthetic construct
  source 1..23: mol_type = other RNA
aagtgtgtgtgtgtgtgtgtgtc

SEQ ID NO: 141 - RNA, 24 nt, synthetic construct
  source 1..24: mol_type = other RNA
aaagtgtgtgtgtgtgtgtgtgtc

SEQ ID NO: 142 - RNA, 25 nt, synthetic construct
  source 1..25: mol_type = other RNA
aaaagtgtgtgtgtgtgtgtgtgtc

SEQ ID NO: 143 - RNA, 26 nt, synthetic construct
  source 1..26: mol_type = other RNA
aaaaagtgtgtgtgtgtgtgtgtgtc

SEQ ID NO: 144 - RNA, 24 nt, synthetic construct
  source 1..24: mol_type = other RNA
cacacacacacacacacacacttt

SEQ ID NO: 145 - RNA, 21 nt, synthetic construct
  source 1..21: mol_type = other RNA
gtgtgtgtgtgtgtgtgtgtg

SEQ ID NO: 146 - RNA, 22 nt, synthetic construct
  source 1..22: mol_type = other RNA
agtgtgtgtgtgtgtgtgtgtg

SEQ ID NO: 147 - RNA, 23 nt, synthetic construct
  source 1..23: mol_type = other RNA
aagtgtgtgtgtgtgtgtgtgtg

SEQ ID NO: 148 - RNA, 24 nt, synthetic construct
  source 1..24: mol_type = other RNA
aaagtgtgtgtgtgtgtgtgtgtg

SEQ ID NO: 149 - RNA, 25 nt, synthetic construct
  source 1..25: mol_type = other RNA
aaaagtgtgtgtgtgtgtgtgtgtg

SEQ ID NO: 150 - RNA, 26 nt, synthetic construct
  source 1..26: mol_type = other RNA
aaaaagtgtgtgtgtgtgtgtgtgtg

SEQ ID NO: 151 - RNA, 24 nt, synthetic construct
  source 1..24: mol_type = other RNA
tacacacacacacacacacacttt

SEQ ID NO: 152 - RNA, 21 nt, synthetic construct
  source 1..21: mol_type = other RNA
gtgtgtgtgtgtgtgtgtgta

SEQ ID NO: 153 - RNA, 22 nt, synthetic construct
  source 1..22: mol_type = other RNA
agtgtgtgtgtgtgtgtgtgta

SEQ ID NO: 154 - RNA, 23 nt, synthetic construct
  source 1..23: mol_type = other RNA
aagtgtgtgtgtgtgtgtgtgta

SEQ ID NO: 155 - RNA, 24 nt, synthetic construct
  source 1..24: mol_type = other RNA
aaagtgtgtgtgtgtgtgtgtgta

SEQ ID NO: 156 - RNA, 25 nt, synthetic construct
  source 1..25: mol_type = other RNA
aaaagtgtgtgtgtgtgtgtgtgta

SEQ ID NO: 157 - RNA, 26 nt, synthetic construct
  source 1..26: mol_type = other RNA
aaaaagtgtgtgtgtgtgtgtgtgta

SEQ ID NO: 158 - RNA, 78 nt, synthetic construct
  source 1..78: mol_type = other RNA
gacgcttaacaaataaacaacaaaaatgagaaaaacaatcatatgtctgttttttctttgatctggttgttaagcgtc

SEQ ID NO: 159 - RNA, 1565 nt, Influenza A virus
  source 1..1565: mol_type = genomic RNA
agcaaaagcagggtagataatcactcactgagtgacatcaaaatcatggcgtcccaaggcaccaaacggtcttacgaacagatggagactgatggagaacgccagaatgccactgaaatcagagcatccgtcggaaaaatgattggtggaattggacgattctacatccaaatgtgcacagaacttaaactcagtgattatgagggacggttgatccaaaacagcttaacaatagagagaatggtgctctctgcttttgacgaaaggagaaataaatacctggaagaacatcccagtgcggggaaggatcctaagaaaactggaggacctatatacagaagagtaaacggaaagtggatgagagaactcatcctttatgacaaagaagaaataaggcgaatctggcgccaagctaataatggtgacgatgcaacggctggtctgactcacatgatgatctggcattccaatttgaatgatgcaacttatcagaggacaagggctcttgttcgcaccggaatggatcccaggatgtgctctctgatgcaaggttcaactctccctaggaggtctggagccgcaggtgctgcagtcaaaggagttggaacaatggtgatggaattggtcaggatgatcaaacgtgggatcaatgatcggaacttctggaggggtgagaatggacgaaaaacaagaattgcttatgaaagaatgtgcaacattctcaaagggaaatttcaaactgctgcacaaaaagcaatgatggatcaagtgagagagagccgggacccagggaatgctgagttcgaagatctcacttttctagcacggtctgcactcatattgagagggtcggttgctcacaagtcctgcctgcctgcctgtgtgtatggacctgccgtagccagtgggtacgactttgaaagagagggatactctctagtcggaatagaccctttcagactgcttcaaaacagccaagtgtacagcctaatcagaccaaatgagaatccagcacacaagagtcaactggtgtggatggcatgccattctgccgcatttgaagatctaagagtattgagcttcatcaaagggacgaaggtggtcccaagagggaagctttccactagaggagttcaaattgcttccaatgaaaatatggagactatggaatcaagtacacttgaactgagaagcaggtactgggccataaggaccagaagtggaggaaacaccaatcaacagagggcatctgcgggccaaatcagcatacaacctacgttctcagtacagagaaatctcccttttgacagaacaaccgttatggcagcattcactgggaatacagaggggagaacatctgacatgaggaccgaaatcataaggatgatggaaagtgcaagaccagaagatgtgtctttccaggggcggggagtcttcgagctctcggacgaaaaggcagcgagcccgatcgtgccttcctttgacatgagtaatgaaggatcttatttcttcggagacaatgcagaggagtacgacaattaaagaaaaatacccttgtttctact

SEQ ID NO: 160 - RNA, 11932 nt, Rabies virus
  source 1..11932: mol_type = genomic RNA
acgcttaacaaccagatcaaagaaaaaacagacagcgtcaatggcagagcaaaaatgtaacacctctacaatggatgccgacaagattgtattcaaagtcaataatcaggtggtctctttgaagcctgagattatcgtggatcaatatgagtacaagtaccctgccatcaaagatttgaaaaagccctgtataactctaggaaaggctcccgatttaaataaagcatacaagtcagttttatcatgcatgagcgccgccaaacttgatcctgacgatgtatgttcctatttggcggcggcaatgcagttttttgaggggacatgtccggaagactggaccagctatggaatcgtgattgcacgaaaaggagataagatcaccccaggttctctggtggagataaaacgtactgatgtagaagggaattgggctctgacaggaggcatggaactgacaagagaccccactgtccctgagcatgcgtccttagtcggtcttctcttgagtctgtataggttgagcaaaatatccgggcaaagcactggtaactataagacaaacattgcagacaggatagagcagatttttgagacagccccttttgttaaaatcgtggaacaccatactctaatgacaactcacaaaatgtgtgctaattggagtactataccaaacttcagatttttggccggaacctatgacatgtttttctcccggattgagcatctatattcagcaatcagagtgggcacagttgtcactgcttatgaagactgttcaggactggtgtcatttactgggttcataaaacaaatcaatctcaccgctagagaggcaatactatatttcttccacaagaactttgaggaagagataagaagaatgtttgagccagggcaggagacagctgttcctcactcttatttcatccacttccgttcactaggcttgagtgggaaatctccttattcatcaaatgctgttggtcacgtgttcaatctcattcactttgtaggatgctatatgggtcaagtcagatccctaaatgcaacggttattgctgcatgtgctcctcatgaaatgtctgttctagggggctatctgggagaggaattcttcgggaaagggacatttgaaagaagattcttcagagatgagaaagaacttcaagaatacgaggcggctgaactgacaaagactgacgtagcactggcagatgatggaactgtcaactctgacgacgaggactacttctcaggtgaaaccagaagtccggaagctgtttatactcgaatcataatgaatggaggtcgactgaagagatcgcacatacggagatatgtctcagtcagttccaatcatcaagctcgtccaaactcattcgccgagtttctaaacaagacatattcgagtgactcataagaagttgaataacaaaatgccggaaatctacggattgtgtatatccatcatgaaaaaaactaacacccctcctttcgaaccaccccaaacatgagcaagatctttgtcaatcctagtgctattagagccggtctggccgatcttgagatggctgaagaaactgttgatctgatcaatagaaatatcgaagacaatcaggctcatctccaaggggaacccatagaagtggacaatctccctgaggatatggggcgacttcacctggatgatggaaaatcgcccaaccctggtgagatggccaaggtgggagaaggcaagtatcgagaggactttcagatggatgaaggagaggatcctagcctcctgttccagtcatacctggacaatgttggagtccaaatagtcagacaaataaggtcaggagagagatttctcaagatatggtcacagaccgtagaagagattatatcctatgtcgcggtcaactttcccaaccctccaggaaagtcttcagaggataaatcaacccagactaccggccgagagctcaagaaggagacaacacccactccttctcagagagaaagccaatcctcgaaagccaggatggcggctcaaactgcttctggccctccagcccttgaatggtcggccaccaatgaagaggatgatctatcagtggaggctgagatcgctcaccagattgcagaaagtttctccaaaaaatataagtttccctctcgatcctcagggatactcttgtataattttgagcaattgaaaatgaaccttgatgatatagttaaagaggcaaaaaatgtaccaggtgtgacccgtttagcccgtgacgggtccaaactccccctaagatgtgtactgggatgggtcgccttggccaactctaagaaattccagttgttagtcgaatccaacaagctgagtaaaatcatgcaagatgacttgaatcgctatacatcttgctaaccgaacctctccactcagtccctctagacaataaagtccgagatgtcctaaagtcaacatgaaaaaaacaggcaacaccactgataaaatgaactttctacgtaagatagtgaaaaattgcagggacgaggacactcaaaaaccctctcccgtgtcagcccctctggatgacgatgacttgtggcttccaccccctgaatacgtcccgctaaaagaacttacaagcaagaagaacaggaggaacttttgtatcaacggaggggttaaagtgtgtagcccgaatggttactcgttcgggatcctgcggcacattctgagatcattcgacgagatatattctgggaatcataggatggtcgggttagtcaaagtagttattggactggctttgtcaggagctccagtccctgagggcatgaactgggtatacaagttgaggagaacccttatcttccagtgggctgattccaggggccctcttgaaggggaggagttggaatactctcaggagatcacttgggatgataatactgagttcgtcggattgcaaataagagtgagtgcaaaacagtgtcatatccggggcagaatctggtgtatcaacatgaactcgagagcaggtcaactatggtctgacatgtctcttcagacacaaaggtccgaagaggacaaagattcctctctgcttctagaataatcagattatatcccgcaaatttatcacttgtttacctctggaggagagaacatatgggctcaactccaacccttgggggcaatataacaaaaaaacatgttatggtgccattaaaccgctgcatttcatcaaagtcaagttaattacctttacattttgatcctcttggatgtgaaaaaaactattaacatccctcaaaagactcaaggaaagatggttcctcaggctctcctgtttgtaccccttctggtttttccattgtgttttgggaaattccctatttacacgataccagacaagcttggtccctggagcccgattgacatacatcacctcagctgcccaaacaatttggtagtggaggacgaaggatgcaccaacctgtcagggttctcctacatggaacttaaagttggatacatctcagccataaaaatgaacgggttcacttgcacaggcgttgtgacggaggctgaaacctacactaacttcgttggttatgtcacaaccacgttcaaaagaaagcatttccgcccaacaccagatgcatgtagagccgcgtacaactggaagatggccggtgaccccagatatgaagagtctctacacaatccgtaccctgactaccactggcttcgaactgtaaaaaccaccaaggagtctctcgttatcatatctccaagtgtggcagatttggacccatatgacagatcccttcactcgagggtcttccctggcgggaattgctcaggagtagcggtgtcttctacctactgctccactaaccacgattacaccatttggatgcccgagaatccgagactagggatgtcttgtgacatttttaccaatagtagagggaagagagcatccaaagggagtgagacttgcggctttgtagatgaaagaggcctatataagtctttaaaaggagcatgcaaactcaagttatgtggagttctaggacttagacttatggatggaacatgggtcgcgatgcaaacatcaaatgaaaccaaatggtgccctcccggtcagttggtgaatttgcacgactttcgctcagacgaaattgagcaccttgttgtagaggagttggtcaagaagagagaggagtgtctggatgcactagagtccatcatgaccaccaagtcagtgagtttcagacgtctcagtcatttaagaaaacttgtccctgggtttggaaaagcatataccatattcaacaagaccttgatggaagccgatgctcactacaagtcagtcagaacttggaatgagatcatcccttcaaaagggtgtttaagagttggggggaggtgtcatcctcatgtaaacggggtatttttcaatggtataatattaggacctgacggcaatgtcttaatcccagagatgcaatcatccctcctccagcaacatatggagttgttggtatcctcggttatcccccttatgcaccccctggcagacccgtctaccgttttcaagaacggtgacgaggctgaggattttgttgaagttcaccttcccgatgtgcacgaacggatctcaggagttgacttgggtctcccgaactgggggaagtatgtattactgagtgcaggggccctgactgccttgatgttgataattttcctgatgacatgctggagaagagtcaatcgatcggaacctacacaacacaatctcagagggacagggagggaggtgtcagtcactccccaaagcgggaagatcatatcttcatgggaatcatacaagagcgggggtgagaccggactgtgagagctggccgtcctttcaacgatccaagtcctgaagatcacctccccttggggggttctttttgaaaaaaaacctgggttcaatagtcctccttgaactccatgcaactgggtagattcaagagtcatgagattttcattaatcctctcagttgatcaagcaagatcatgtagattctcataataggggagatcttctagcagtttcagtgactaacggtgctttcattctccaggaactgacaccaacagttgtagacaaatcacggggtgtctcaggtgattctgcgcttgggcacagacaaaggtcatggtgtgttccatgatagcggactcaggatgagttaattgagagaggcaatcttcctcccgtgaaggacacaagcagtagctcacaatcatctcgtgtttcagcaaagtgtgcataattataaagtgctgggtcatctaagcttttcagtcgagaaaaaaacagtagatcagaagaacaactggcaacacttctcatcctgagacctacttcaagatgctcgatcctggagaggtctatgatgaccctattgacccaatcgagttagaggctgaacccagaggaacccccactgtccccaacatcttgaggaactctgactacaatctcaactctcctttgatagaagatcctgctagactaatgttagaatggttaaaaacagggaatagaccttatcggatgactctaacagacaattgctccaggtctttcagagttttgaaagattatttcaagaaggtagatttgggttccctcaaggtgggcggaatggctgcacagtcaatgatttctctctggttatatggtgcccactctgaatccaacaggagccggagatgtataacagacttggcccatttctattccaagtcgtcccccatagagaagctgttaaatctcacgctaggaaatagagggctgagaatccccccagagggagtgttaagttgccttgagagggttgattatgataatgcatttggaaggtatcttgccaacacgtattcctcttacttgttcttccatgtaatcaccttatacatgaacgccctagactgggatgaagaaaagaccatcctagcattatggaaagatttaacctcagtggacatcgggaaggacttggtaaagttcaaagaccaaatatggggactgctgatcgtgacaaaggactttgtttactcccaaagttccaattgtctttttgacagaaactacacacttatgctaaaagatcttttcttgtctcgcttcaactccttaatggtcttactttctcccccagagccccgatactcagatgacttgatatctcagctatgccagctgtacattgctggggatcaagtcttgtctatgtgtggaaactccggctatgaagtcatcaaaatattggagccatatgtcgtgaatagtttagtccagagagcagaaaagtttaggcctctcattcattccttgggagactttcctgtatttataaaagacaaggtaagtcaactcgaagagacgttcggttcctgtgcaagaaggttctttagggctctggatcaattcgacaacatacatgacttggtttttgtgtatggctgttacaggcattgggggcacccatatatagattatcgaaagggtctgtcaaaactatatgatcaggttcacattaaaaaagtgatagataagtcctaccaggagtgcttagcaagcgacctagccaggaggatccttagatggggttttgataagtactccaagtggtatctggattcacgattcctagcccgagaccaccccttgactccttatatcaaaacccaaacatggccacccaaacatattgtagatttggtgggggatacatggcacaagctcccgatcacgcaaatctttgagattcctgaatcaatggatccatcagaaatattggatgacaaatcacattctttcaccagaacgagactagcttcttggctgtcagaaaaccgaggggggcctgttcctagcgaaaaagttattatcacggccctgtctaagccgcctgtcaatccccgagagtttctgaagtctatagacctcggaggattgccagatgaagacttgataattggcctcaagccaaaggaacgggaattgaagattgaaggtcgattctttgctctaatgtcatggaatctaagattgtattttgtcatcactgaaaaactcttggccaactacatcttgccactttttgacgcgctgactatgacagacaacctgaacaaggtgtttaaaaagctgatcgacagggtcaccgggcaagggcttctggactattcaagggtcacatatgcatttcacctggactatgaaaagtggaacaaccatcaaagattagagtcaacagaggatgtattttctgtcctagatcaagtgtttggattgaagagagtgttttctagaacacacgagttttttcagaagtcctggatctattattcagacagatcagacctcatcgggttacgggaggatcaaatatactgcttagatgcgtccaacggcccaacctgttggaatggccaggatggcgggctagaaggcttacggcagaagggctggagtctagtcagcttattgatgatagatagagaatctcaaatcaggaacacaagaaccaaagtactagctcaaggagacaaccaggttttatgtccgacatatatgttgtcgccagggctatctcaagaggggctcctctatgaattggagagcatatcaaggaatgcattttcgatatacagagccgtcgaggaaggggcatctaaactagggctgatcatcaagaaagaagagaccatgtgtagttatgacttcctcatctatggaaaaacccctttgtttagaggtaacatattggtgcctgagtccaaaagatgggccagagtctcttgcgtctctaatgaccaaatagtcaacctcgccaatataatgtcgacagtgtccaccaacgcgctaacagtggcacaacactctcaatctttgatcaaaccgatgagggattttctgctcatgtcagtacaggcagtctttcactacctgctatttagcccaatcttaaagggaagagtttacaagattctgagcgctgaaggggagagctttctcctagccatgtcaaggataatctatctagatccttctttgggaggggtatctggaatgtccctcggaagattccatatacgacagttctcagaccctgtctctgaagggttatccttctggagagagatctggttaagctcccacgagtcctggattcacgcgttgtgtcaagaggctggaaacccagatcttggagagagaacactcgagagcttcactcgccttctagaagatcctaccaccttaaatatcagaggaggggccagtcctaccattctactcaaggatgcaatcagaaaggctttatatgacgaggtggacaaggtggagaactcagagtttcgagaggcaatcctgttgtccaagacccatagagataattttatactcttcttaacatctgttgagcctctgtttcctcgatttctcagtgagctattcagttcgtcttttttgggaatccccgagtcaatcattggactgatacaaaactcccgaacgataagaaggcagtttagaaagagtctctcaaaaactttagaagaatccttctacaactcagagatccacgggattagtcggatgacccagacacctcagagggttgggggggtgtggccttgctcttcagagagggcagatctacttagggagatctcttggggaagaaaagtggtaggcacgacagttcctcacccttctgagatgttggggttacttcccaagtcctctatttcttgcacttgtggagcaacaggaggaggcaatcctagagtttctgtatcagtactcccgtcttttgatcagtcatttttttgcacggggcccctaaaggggtacttgggctcgtccacctctatgtcgacccagctattccatgcatgggaaaaagtcactaatgttcatgtggtgaagagagctctatcgttaaaagaatctataaactggttcattactagagattccaacttggctcaaactctaattaggaacattgtgtctctgacaggccctgatttccctctagaggaggcccctgttttcaaaaggacggggtcagccttgcataggttcaagtctgccagatacagcgaaggagggtattcttctgtatgcccgaacctcctctctcatatttctgttagtacagacaccatgtctgatttgacccaagacgggaagaactacgatttcatgttccagccattgatgctttatgcacagacatggacatcagagctggtacagagagacacaaggctaagagactctacgtttcattggcacctccaatgcaacaggtgtgtgagacccattgacgacgtgaccctggagacctctcagatcttcgagtttccggatgtgtcgaaaagaatatccagaatggtttctggggctgtgcctcacttccagaggcttcccgatatccgtctgagaccaggagattttgaatctctaagcggtagagaaaagtctcaccatatcggatcagctcaggggctcttatactcaatcttagtggcaattcacgactcaggatacaatgatggaaccatcttccctgtcaacatatacggcaaggtttcccctagagactatttgagagggctcgcaaggggagtattgataggatcctcgatttgcttcttgacgagaatgacaaatatcaatattaatagacctcttgaattgatctcaggggtaatctcatatattctcctgaggctagataaccatccctccttgtacataatgctcagagaaccgtcttttagagaagagatattttctatccctcagaaaatccccgccgcttatccaaccactatgaaagaaggcaacagatcaatcttgtgttatctccaacatgtgctacgctatgagcgagaggtaatcacggcgtctccagagaatgactggctatggatcttttcagactttagaagtgccaaaatgacgtacctaaccctcattacttaccagtctcatcttctactccagagggttgagagaaacctatctaagagtatgagagataacctgcgacaattgagttccttgatgaggcaggtgctgggcgggcacggagaagataccttagagtcagacgacaacattcaacgactactaaaagactctttacgaaggacaagatgggtggatcaagaggtgcgccatgcagctagaaccatgactggagattacagccccaacaagaaggtgtcccgtaaggtaggatgttcagaatgggtctgctctgctcaacaggttgcagtctctacctcagcaaacccggcccctgtctcggagcttgacataagggccctctctaagaggttccagaaccctttgatctcgggcttgagagtggttcagtgggcaaccggtgctcattataagcttaagcctattctagatgatctcaatgttttcccatctctctgccttgtagttggggacgggtcaggggggatatcaagggcagtcctcaacatgtttccagatgccaagcttgtgttcaacagtcttttagaggtgaatgacctgatggcttccggaacacatccactgcctccttcagcaatcatgaggggaggaaatgatatcgtctccagagtgatagattttgactcaatctgggaaaaaccgtccgacttgagaaacttggctacctggaaatacttccagtcagtccaaaagcaggtcaacatgtcctatgacctcattatttgcgatgcagaagttactgacattgcatctatcaaccggataaccctgttaatgtccgattttgcattgtctatagatggaccactctatttggtcttcaaaacttatgggactatgctagtaaatccaaactacaaggctattcaacacctgtcaagagcgttcccctcggtcacagggtttatcacccaagtaacttcgtctttttcatctgagctctaccttcgattctccaaacgagggaagtttttcagagatgctgagtacttgacctcttccacccttcgagaaatgagccttgtgttattcaattgtagcagccccaagagtgagatgcagagagctcgttccttgaactatcaggatcttgtgagaggatttcctgaagaaatcatatcaaatccttacaatgagatgatcataactctgattgacagtgatgtagaatcttttctagtccacaagatggtggatgatcttgagttacagaggggaactctgtctaaagtggctatcattatagccatcatgatagttttctccaacagagtcttcaacgtttccaaacccctaactgaccccttgttctatccaccgtctgatcccaaaatcctgaggcacttcaacatatgttgcagtactatgatgtatctatctactgctttaggtgacgtccctagcttcgcaagacttcacgacctgtataacagacctataacttattacttcagaaagcaagtcattctagggaacgtttatctatcttggagttggtccaacgacacctcagtgttcaaaagggtagcctgtaattctagcctgagtctgtcatctcactggatcaggttgatttacaagatagtgaagactaccagactcgttggcagcatcaaggatctatccggagaagtggaaagacaccttcataggtacaacaggtggatcaccctagagaatatcagatctagatcatccctactagactacagttgcctgtgcatcggatactcctggaagcctgcccatgctaagactcttgtgtgatgtattttgaaaaaaacaagatcttaaatctgaacctctagttgtttgattgtttttctcatttttgttgtttatttgttaagcgt

SEQ ID NO: 161 - RNA, 15186 nt, unidentified
  source 1..15186: mol_type = unassigned RNA
accaaacagagaatcggtgagttacgataaaaggcgaaggagcaattgaagtcgcacgggtagaaggtgtgaatctcgagtgcgagcccgaagcacaaactcgaggaagccttctgccaacatgtcttccgtattcgacgagtacgaacagctcctcgcggctcagactcgccccaatggagctcatggagggggggagaaagggagtaccttaaaagtagacgtcccggtattcactcttaacagtgatgacccagaagataggtggagctttgtggtattctgcctccggattgctgttagcgaagatgccaacaaaccactcaggcaaggtgctctcatatctcttttatgctcccactcacaggtaatgaggaaccatgttgcccttgcagggaaacagaatgaagccacattggccgtgcttgagattgatggctttgccaacggcacgccccagttcaacaataggagtggagtgtctgaagagagagcacagagatttgcgatgatagcaggatctctccctcgggcatgcagcaacggcaccccgttcgtcacagccggggctgaagatgatgcaccagaagacatcaccgataccctggagaggatcctctctatccaggctcaagtatgggtcacagtagcaaaagccatgactgcgtatgagactgcagatgagtcggaaacaaggcgaatcaataagtatatgcagcaaggcagggtccaaaagaaatacatcctctaccccgtatgcaggagcacaatccaactcacgatcagacagtctcttgcagtccgcatctttttggttagcgagctcaagagaggccgcaacacggcaggtggtacctctacttattataacctagtaggggacgtagactcatatatcaggaataccgggcttactgcattcttcttgacactcaagtacggaatcaacaccaagacatcagcccttgcacttagtagcctctcaggcgacatccagaagatgaagcagctcatgcgtttgtatcggatgaaaggagataatgcgccgtacatgacattacttggtgatagtgaccagatgagctttgcgcctgccgagtatgcacaactttactcctttgccatgggtatggcatcagtcctagataaaggtactgggaaataccaatttgccaaggactttatgagcacatcattctggagacttggagtagagtacgctcaggctcagggaagtagcattaacgaggatatggctgccgagctaaagctaaccccggcagcaaggaggggcctggcagctgctgcccaacgagtctccgaggtgaccagcagcatagacatgcctactcaacaagtcggagtcctcactgggcttagcgaggggggatcccaagccctacaaggcggatcgaatagatcgcaagggcaaccagaagccggggatggggagacccaattcctggatctgatgagagcggtagcaaatagcatgagggaggcgccaaactctgcacagggcactccccaatcggggcctcccccaactcctgggccatcccaagataacgacaccgactgggggtattgattgacaaaacccagcctgcttctacaagaacatcccaatgctctcacccgtagtcgacccctcgatttgcggctctatatgaccacaccctcaaacaaacatccccctctttcctccctccccctgctgtacaactccgcacgccctaggcaacagaggcacaatgcggctcactaacaatcaaaacagagccgagggaattagaaaaaagtacgggtagaagagggatattcagagatcagggcaagtctcccgagtctctgctctctcctctacctgatagaccaggacaaacatggccacctttacagatgcagagatcgacgagctatttgagacaagtggaactgtcattgacaacataattacagcccagggtaaaccagcagagactgttggaaggagtgcaatcccacaaggcaagaccaaggtgctgagcgcagcatgggagaagcatgggagcatccagccaccggccagtcaagacaaccccgatcgacaggacagatctgacaaacaaccatccacacccgagcaaacgaccccgcatgacagcccgccggccacatccgctgaccagccccccacccaggccacagacgaagccgtcgacacacagctcaggaccggagcaagcaactctctgctgttgatgcttgacaagctcagcaataaatcgtccaatgctaaaaagggcccatggtcgagcccccaagaggggaatcaccaacgtccgactcaacagcaggggagtcaacccagtcgcggaaacagtcaggaaagaccgcagaaccaagtcaaggccgcccctggaaaccagggcacagacgtgaacacagcatatcatggacaatgggaggagtcacaactatcagctggtgcaacccctcatggtctccgatcaaagcagagccaaaacaatacccctgtttctgcggatcatttccacccacctgtagactttgtgcaagcgatgatgtctattatggaggggatttcccaaagagtaagtaaggttgcctatcaggtagatcttgtttttaaacagacatcctccatccctatgatggggtccgaaatccaacagctgaaaacatttgttgcagtcatggaagccaacttgggaatgatgaagattttggatcccggttgtgccaacatttcatctttgagtgatctacgggcagttgcccgatctcacccggttttagtttcaggccctggagacccatctccctatgtgatacaaggaggcgaaatggcacttaataaactttcgcaaccagtgccacatccatctgaattgattaaacccgccactgcatgcgggcctgatataggagtggagagggacactgtccgtgcattgatcatgtcacgcccaatgcacccgagttcttcagccaagctcctaagcaagttagatgcagccgggtcgatcgaggaaatcaggaaaatcaagcgccttgctctaaatggctaattactactgccacacgtagcgggtccctgtccactcggcatcacacggaatctgcaccgagttcccccccgcagacccaaggtccaactctccaagcggcaatcctctctcgcttcctcagccccactgaatgatcgcgcaaccgcaattaatctagctacattaaggattaagaaaaaatacgggtagaattggagtgccccaattgtgccaagatggactcatctaggacaattgggctgtactttgattctgcccattcttctagcaacctgttagcatttccgatcgtcctacaagacacaggagatgggaagaagcaaatcgccccgcaatataggatccagcgccttgacttgtggactgatagtaaggaagactcagtattcatcaccacctatggattcatctttcaagttgggaatgaagaagccactgtcggcattatcgatgataaacccaagcgcgagttactttccgctgcgatgctctgcctaggaagcgtcccaaataccggagaccttattgagctggcaagggcctgtctcactatgatggtcacatgcaagaagagtgcaactaatactgagagaatggttttctcagtagtgcaggcaccccaagtgctgcaaagctgtagggttgtggcaaacaaatactcatcagtgaatgcagtcaagcacgtgaaagcgccagagaagatccccgggagtggaaccctagaatacaaggtgaactttgtctccttgactgtggtaccgaagaaggatgtctacaagatcccagctgcagtattgaagatttctggctcgagtctgtacaatcttgcgctcaatgtcactattaatgtggaggtagacccgaggagtcctttggttaaatctctgtctaagtctgacagcggatactatgctaacctcttcttgcatattggacttatgaccaccgtagataggaaggggaagaaagtgacatttgacaagctggaaaagaaaataaggagccttgatctatctgtcgggctcagtgatgtgctcgggccttccgtgttggtaaaagcaagaggtgcacggactaagcttttggcacctttcttctctagcagtgggacagcctgctatcccatagcaaatgcttctcctcaggtggccaagatactctggagtcaaaccgcgtgcctgcggagcgttaaaatcattatccaagcaggtacccaacgcgctgtcgcagtgaccgctgaccacgaggttacctctactaagctggagaaggggcacacccttgccaaatacaatccttttaagaaataagctgcgtctctgagattgcgctccgcccactcacccagatcatcatgacacaaaaaactaatctgtcttgattatttacagttagtttacctgtccatcaagttagaaaaaacacgggtagaagactctggatcccggttggcgccctccaggtgcaggatgggctccagaccttttaccaagaacccagcacctatgatgctgactatccgggtcgcgctggtattgagttgcatctgtccggcaaactccattgatggcaggccttttgcagctgcaggaattgtggttacaggagacaaagcagtcaacatatacacctcatcccagacaggatcaatcatagttaagctcctcccgaatctgcccaaggataaggaggcatgtgcgaaagcccccttggatgcatacaacaggacattgaccactttgctcaccccccttggtgactctatccgtaggatacaagagtctgtgactacatctggaggggggagacaggggcgccttataggcgccattattggcggtgtggctcttggggttgcaactgccgcacaaataacagcggccgcagctctgatacaagccaaacaaaatgctgccaacatcctccgacttaaagagagcattgccgcaaccaatgaggctgtgcatgaggtcactgacggattatcccaactagcagtggcagttgggaagatgcagcagtttgttaatgaccaatttaataaaacagctcaggaattagactgcataaaaattgcacagcaagttggtgtagagctcaacctgtacctaaccgaattgactacagtattcggaccacaaatcacttcacctgccttaaacaagctgactattcaggcactttacaatctagctggtgggaatatggattacttattgactaagttaggtatagggaacaatcaactcagctcattaatcggtagcggcttaatcaccggtaaccctattctatacgactcacagactcaactcttgggtatacaggtaactctaccttcagtcgggaacctaaataatatgcgtgccacctacttggaaaccttatccgtaagcacaaccaggggatttgcctcggcacttgtcccaaaagtggtgacacaggtcggttctgtgatagaagaacttgacacctcatactgtatagaaactgacttagatttatattgtacaagaatagtaacgttccctatgtcccctggtatttactcctgcttgagcggcaatacatcggcctgtatgtactcaaagaccgaaggcgcacttactacaccatatatgactatcaaaggctcagtcatcgctaactgcaagatgacaacatgtagatgtgtaaaccccccgggtatcatatcgcaaaactatggagaagccgtgtctctaatagataaacaatcatgcaatgttttatccttaggcgggataactttaaggctcagtggggaattcgatgtaacttatcagaagaatatctcaatacaagattctcaagtaataataacaggcaatcttgatatctcaactgagcttgggaatgtcaacaactcgatcagtaatgctttgaataagttagaggaaagcaacagaaaactagacaaagtcaatgtcaaactgaccagcacatctgctctcattacctatatcgttttgactatcatatctcttgtttttggtatacttagcctgattctagcatgctacctaatgtacaagcaaaaggcgcaacaaaagaccttattatggcttgggaataataccctagatcagatgagagccactacaaaaatgtgaacacagatgaggaacgaaggtttccctaatagtaatttgtgtgaaagttctggtagtctgtcagttcggagagttaagaaaaaactaccggttgtagatgaccaaaggacgatatacgggtagaacggtaagagaggccgcccctcaattgcgagccagacttcacaacctccgttctaccgcttcaccgacaacagtcctcaatcatggaccgcgccgttagccaagttgcgttagagaatgatgaaagagaggcaaaaaatacatggcgcttgatattccggattgcaatcttattcttaacagtagtgaccttggctatatctgtagcctcccttttatatagcatgggggctagcacacctagcgatcttgtaggcataccgactaggatttccagggcagaagaaaagattacatctacacttggttccaatcaagatgtagtagataggatatataagcaagtggcccttgagtctccattggcattgttaaatactgagaccacaattatgaacgcaataacatctctctcttatcagattaatggagctgcaaacaacagcgggtggggggcacctattcatgacccagattatataggggggataggcaaagaactcattgtagatgatgctagtgatgtcacatcattctatccctctgcatttcaagaacatctgaattttatcccggcgcctactacaggatcaggttgcactcgaataccctcatttgacatgagtgctacccattactgctacacccataatgtaatattgtctggatgcagagatcactcacactcatatcagtatttagcacttggtgtgctccggacatctgcaacagggagggtattcttttctactctgcgttccatcaacctggacgacacccaaaatcggaagtcttgcagtgtgagtgcaactcccctgggttgtgatatgctgtgctcgaaagccacggagacagaggaagaagattataactcagctgtccctacgcggatggtacatgggaggttagggttcgacggccaatatcacgaaaaggacctagatgtcacaacattattcggggactgggtggccaactacccaggagtagggggtggatcttttattgacagccgcgtatggttctcagtctacggagggttaaaacccaattcacccagtgacactgtacaggaagggaaatatgtgatatacaagcgatacaatgacacatgcccagatgagcaagactaccagattcgaatggccaagtcttcgtataagcctggacggtttggtgggaaacgcatacagcaggctatcttatctatcaaagtgtcaacatccttaggcgaagacccggtactgactgtaccgcccaacacagtcacactcatgggggccgaaggcagaattctcacagtagggacatcccatttcttgtatcagcgagggtcatcatacttctctcccgcgttattatatcctatgacagtcagcaacaaaacagccactcttcatagtccttatacattcaatgccttcactcggccaggtagtatcccttgccaggcttcagcaagatgccccaactcgtgtgttactggagtctatacagatccatatcccctaatcttctatagaaaccacaccttgcgaggggtattcgggacaatgcttgatggtgaacaagcaagacttaaccctgcgtctgcagtattcgatagcacatcccgcagtcgcataactcgagtgagttcaagcagcatcaaagcagcatacacaacatcaacttgttttaaagtggtcaagaccaataagacctattgtctcagcattgctgaaatatctaatactctcttcggagaattcagaatcgtcccgttactagttgagatcctcaaagatgacggggttagagaagccaggtctggctagttgagtcaactatgaaagagttggaaagatggcattgtatcacctatcttctgcgacatcaagaatcaaaccgaatgccggcgcgtgctcgaattccatgtcgccagttgaccacaatcagccagtgctcatgcgatcagattaagccttgtcaatagtctcttgattaagaaaaaatgtaagtggcaatgagatacaaggcaaaacagctcatggtaaataatacgggtaggacatggcgagctccggtcctgaaagggcagagcatcagattatcctaccagagtcacacctgtcttcaccattggtcaagcacaaactactctattattggaaattaactgggctaccgcttcctgatgaatgtgacttcgaccacctcattctcagccgacaatggaaaaaaatacttgaatcggcctctcctgatactgagagaatgataaaactcggaagggcagtacaccaaactcttaaccacaattccagaataaccggagtactccaccccaggtgtttagaagaactggctaatattgaggtccctgattcaaccaacaaatttcggaagattgagaagaagatccaaattcacaacacgagatatggagaactgttcacaaggctgtgtacgcatatagagaagaaactgctggggtcatcttggtctaacaatgtcccccggtcagaggagttcagcagcattcgtacggatccggcattctggtttcactcaaaatggtccacagccaagtttgcatggctccatataaaacagatccagaggcatctgattgtggcagctaggacaaggtctgcggccaacaaattggtgatgctaacccataaggtaggccaagtctttgtcactcctgaacttgttgttgtgacgcatacgaatgagaacaagttcacatgtcttacccaggaacttgtattgatgtatgcagatatgatggagggcagagatatggtcaacataatatcaaccacggcggtgcatctcagaagcttatcagagaaaattgatgacattttgcggttaatagacgctctggcaaaagacttgggtaatcaagtctacgatgttgtatcactaatggagggatttgcatacggagctgtccagctactcgagccgtcaggtacatttgcgggagatttcttcgcattcaacctgcaggagcttaaagacattctaattggcctcctccccaatgatatagcagaatccgtgactcatgcaatcgctactgtattctctggtttagaacagaatcaagcagctgagatgttgtgcctgttgcgtctgtggggtcacccactgcttgagtcccgtattgcagcaaaggcagtcaggagccaaatgtgcgcaccgaaaatggtagactttgatatgatccttcaggtactgtctttcttcaagggaacaatcatcaacggatacagaaagaagaatgcaggtgtgtggccgcgagtcaaagtggatacaatatatgggaaggtcattgggcaactacatgcagattcagcagagatttcacacgatatcatgttgagagagtataagagtttatctgcacttgaatttgagccatgtatagaatacgaccctgtcactaacctgagcatgttcctaaaagacaaggcaatcgcacaccccaacgataattggcttgcctcgtttaggcggaaccttctctccgaagaccagaagaaacatgtaaaggaagcgacttcgactaaccgcctcttgatagagtttttagagtcaaatgattttgatccatataaagagatggaatatctgacgacccttgagtaccttagagatgacaatgtggcagtatcatactcgctcaaagagaaggaagtgaaagttaatggacggatcttcgctaagctgacaaagaagttaaggaactgtcaggtgatggcggaagggatcctagccgatcagattgcacctttctttcagggaaatggagtcattcaggatagcatatccttgaccaagagtatgctagcgatgagtcaactgtcttttaacagcaataagaaacgtatcactgactgtaaagaaagagtatcttcaaaccgcaatcatgatccgaaaagcaagaaccgtcggagagttgcaaccttcataacaactgacctgcaaaagtactgtcttaattggagatatcagacgatcaaattgttcgctcatgccatcaatcagttgatgggcctacctcatttcttcgagtggattcacctaagactgatggacactacgatgttcgtaggagaccctttcaatcctccaagtgaccctactgactgtgacctctcaagagtccctaatgatgacatatatattgtcagtgccagagggggtatcgaaggattatgccagaagctatggacaatgatctcaattgctgcaatccaacttgctgcagctagatcgcattgtcgtgttgcctgtatggtacagggtgataatcaagtaatagcagtaacgagagaggtaagatcagatgactctccggagatggtgttgacacagttgcatcaagccagtgataatttcttcaaggaattaatccatgtcaatcatttgattggccataatttgaaggatcgtgaaaccatcaggtcagacacattcttcatatacagcaaacgaatcttcaaagatggagcaatcctcagtcaagtcctcaaaaattcatctaaattagtgctagtgtcaggtgatctcagtgaaaacaccgtaatgtcctgtgccaacattgcctctactgtagcacggctatgcgagaacgggcttcccaaagacttctgttactatttaaactatataatgagttgtgtgcagacatactttgactctgagttctccatcaccaacaattcgcaccccgatcttaatcagtcgtggattgaggacatctcttttgtgcactcatatgttctgactcctgcccaattagggggactgagtaaccttcaatactcaaggctctacactagaaatatcggtgacccggggactactgcttttgcagagatcaagcgactagaagcagtgggactactgagtcctaacattatgactaatatcttaactaggccgcctgggaatggagattgggccagtctgtgcaacgacccatactctttcaattttgagactgttgcaagcccaaatattgttcttaagaaacatacgcaaagagtcctatttgaaacttgttcaaatcccttattgtctggagtgcacacagaggataatgaggcagaagagaaggcattggctgaattcttgcttaatcaagaggtgattcatccccgcgttgcgcatgccatcatggaggcaagctctgtaggtaggagaaagcaaattcaagggcttgttgacacaacaaacactgtaattaagattgcgcttactaggaggccattaggcatcaagaggctgatgcggatagtcaattattctagcatgcatgcaatgctgtttagagacgatgttttttcctctagtagatccaaccaccccttagtctcttctaatatgtgttctctgacactggcagactatgcacggaatagaagctggtcacctttgacgggaggcaggaaaatactgggtgtatctaatcctgatacgatagaactcgtagagggtgagattcttagtgtaagcggagggtgtacaagatgtgacagcggagatgaacaatttacttggttccatcttccaagcaatatagaattgaccgatgacaccagcaagaatcctccgatgagggtaccatatctcgggtcaaagacacaggagaggagagctgcctcacttgcgaaaatagctcatatgtcgccacatgtgaaggctgccctaagggcatcatccgtgttgatctgggcttatggggataatgaagtaaattggactgctgctctcacgattgcaaaatctcggtgtaatgtaaacttagagtatcttcggttactgtcccctttacccacggctgggaatcttcaacatagactagatgatggtataactcagatgacattcacccctgcatctctctacaggtgtcaccttacattcacatatccaatgattctcaaaggctgttcactgaagaaggagtcaaagaggggaatgtggtttaccaacagagtcatgctcttgggtttatctctaatcgaatcgatctttccaatgacaacaaccagaacatatgatgagatcacactgcacctacatagtaaatttagttgctgtatcagggaagcacctgttgcggttcctttcgagctacttggggtggcaccggaactgaggacagtgacctcaaataagtttatgtatgatcctagccctgtatcggagggagactttgcgagacttgacttagctatcttcaagagttatgagcttaatctggagtcatatcccacgatagagctaatgaacattctttcaatatccagcgggaagttgattggccagtctgtggtttcttatgatgaagatacctccataaagaatgatgccataatagtgtatgacaatacccgaaattggatcagtgaagctcagaattcagatgtggtccgcctatttgaatatgcagcacttgaagtgctcctccaccgttcttaccaactctattacctgagagtaagaggcctagacaatattgtcttatatatgggtgatttatacaagaatatgccaggaattctactttccaacattgcagctacaatatctcatcctgtcattcattcaaggttacatgcagtgggcctggtcaaccatgacggatcacaccaacttgcagatacggattttatcgaaatgtctgcaaaactgttagtatcttgcacccgacgtgtgatctccggcttatattcaggaaataagtatgatctgctgttcccatctgtcttagatgataacctgaatgagaagatgcttcagctgatatcccggttatgctgtctgtacacggtactctttgctacaacaagagaaatcccgaaaataagaggcttaactgcagaagagaaatgttcaatactcactgagtatttactgtcggatgctgtgaaaccattacttagccccgatcaagtgagctctatcatgtctcctaacataattacattcccagctaatctgtactacatgtctcggaagagcctcaatttgatcagggaaagggaggacagggatactatcctggcgttgttgttcccccaagagccattattagagttcccttctgtgcaagatattggtgctcgagtgaaagatccattcacccgacaacctgcggcatttttgcaagagttagatttgagtgctccagcaaggtatgacgcattcacacttagtcagattcatcctgaactcacatctccaaatccggaggaagactacttagtacgatacttgttcagagggatagggactgcatcttcctcttggtataaggcatcccatctcctttctgtacccgaggtaagatgtgcaagacacgggaactccttatacttggctgaaggaagcggagccatcatgagtcttcttgaactgcatgtaccacatgaaactatctattacaatacgctcttttcaaatgagatgaaccccccgcaacgacatttcgggccgaccccaactcagtttttgaattcggttgtttataggaatctacaggcggaggtaacatgcaaggatggatttgtccaagagttccgtccattatggagagaaaatacagaggaaagtgacctgacctcagataaagcagtggggtatattacatctgcagtaccctacagatctgtatcattgctgcattgtgacattgaaattcctccagggtccaatcaaagcttactagatcaactagctatcaatttatctctgattgccatgcattctgtaagggagggcggggtagtaatcatcaaagtgttgtatgcaatgggatactactttcatctactcatgaacttgtttgctccgtgttccacaaaaggatatattctctctaatggttatgcatgtcgaggggatatggagtgttacctggtatttgtcatgggttacctgggcgggcctacatttgtacatgaggtggtgaggatggcaaaaactctggtgcagcggcacggtacgctcttgtctaaatcagatgagatcacactgaccaggttattcacctcacagcggcagcgtgtgacagacatcctatccagtcctttaccaagattaataaagtacttgaggaagaatattgacactgcgctgattgaagccgggggacagcccgtccgtccattttgtgcggaaagtttggtgagcacgctagcgaacataactcagataacccagattatcgctagtcacattgacacagtcatccggtctgtgatatatatggaagctgagggtgatctcgctgacacagtatttctatttaccccttacaatctctctactgacgggaaaaagaggacatcacttaaacagtgcacgagacagatcctagaggttacaatactaggtcttagagtcgaaaatctcaataaaataggcgatataatcagcctagtgcttaaaggcatgatctccatggaggaccttatcccactaaggacatacttgaagcatagtacctgccctaaatatttgaaggctgtcctaggtattaccaaactcaaagaaatgtttacagacacttctgtactgtacttgactcgtgctcaacaaaaattctacatgaaaactataggcaatgcagtcaaaggatattacagtaactgtgactcctaacgaaaatcacatattaataggctccttttttggccaattgtattcttgttgatttaattatattatgttagaaaaaagttgaactctgactccttaggactcgaattcgaactcaaataaatgtctttaaaaaaggttgcgcacaattattcttgagtgtagtctcgtcattcaccaaatctttgtttggt

SEQ ID NO: 162 - RNA, 11161 nt, Vesicular stomatitis Indiana virus
  source 1..11161: mol_type = genomic RNA
acgaagacaaacaaaccattattatcattaaaaggctcaggagaaactttaacagtaatcaaaatgtctgttacagtcaagagaatcattgacaacacagtcatagttccaaaacttcctgcaaatgaggatccagtggaatacccggcagattacttcagaaaatcaaaggagattcctctttacatcaatactacaaaaagtttgtcagatctaagaggatatgtctaccaaggcctcaaatccggaaatgtatcaatcatacatgtcaacagctacttgtatggagcattaaaggacatccggggtaagttggataaagattggtcaagtttcggaataaacatcgggaaagcaggggatacaatcggaatatttgaccttgtatccttgaaagccctggacggcgtacttccagatggagtatcggatgcttccagaaccagcgcagatgacaaatggttgcctttgtatctacttggcttatacagagtgggcagaacacaaatgcctgaatacagaaaaaagctcatggatgggctgacaaatcaatgcaaaatgatcaatgaacagtttgaacctcttgtgccagaaggtcgtgacatttttgatgtgtggggaaatgacagtaattacacaaaaattgtcgctgcagtggacatgttcttccacatgttcaaaaaacatgaatgtgcctcgttcagatacggaactattgtttccagattcaaagattgtgctgcattggcaacatttggacacctctgcaaaataaccggaatgtctacagaagatgtaacgacctggatcttgaaccgagaagttgcagatgaaatggtccaaatgatgcttccaggccaagaaattgacaaggccgattcatacatgccttatttgatcgactttggattgtcttctaagtctccatattcttccgtcaaaaaccctgccttccacttctgggggcaattgacagctcttctgctcagatccaccagagcaaggaatgcccgacagcctgatgacattgagtatacatctcttactacagcaggtttgttgtacgcttatgcagtaggatcctctgccgacttggcacaacagttttgtgttggagataacaaatacactccagatgatagtaccggaggattgacgactaatgcaccgccacaaggcagagatgtggtcgaatggctcggatggtttgaagatcaaaacagaaaaccgactcctgatatgatgcagtatgcgaaaagagcagtcatgtcactgcaaggcctaagagagaagacaattggcaagtatgctaagtcagaatttgacaaatgaccctataattctcagatcacctattatatattatgctacatatgaaaaaaactaacagatatcatggataatctcacaaaagttcgtgagtatctcaagtcctattctcgtctggatcaggcggtaggagagatagatgagatcgaagcacaacgagctgaaaagtccaattatgagttgttccaagaggatggagtggaagagcatactaagccctcttattttcaggcagcagatgattctgacacagaatctgaaccagaaattgaagacaatcaaggtttgtatgcacaggatccagaagctgagcaagttgaaggctttatacaggggcctttagatgactatgcagatgaggaagtggatgttgtatttacttcggactggaaaccacctgagcttgaatctgacgagcatggaaagaccttacggttgacatcgccagagggtttaagtggagagcagaaatcccagtggctttcgacgattaaagcagtcgtgcaaagtgccaaatactggaatctggcagagtgcacatttgaagcatcgggagaaggggtcattatgaaggagcgccagataactccggatgtatataaggtcactccagtgatgaacacacatccgtcccaatcagaagcagtatcagatgtttggtctctctcaaagacatccatgactttccaacccaagaaagcaagtcttcagcctctcaccatatccttggatgaattgttctcatctagaggagagttcatctctgtcggaggtgacggacgaatgtctcataaagaggccatcctgctcggcctgagatacaaaaagttgtacaatcaggcgagagtcaaatattctctgtagactatgaaaaaaagtaacagatatcacgatctaagtgttatcccaatccattcatcatgagttccttaaagaagattctcggtctgaaggggaaaggtaagaaatctaagaaattagggatcgcaccacccccttatgaagaggacactagcatggagtatgctccgagcgctccaattgacaaatcctattttggagttgacgagatggacacctatgatccgaatcaattaagatatgagaaattcttctttacagtgaaaatgacggttagatctaatcgtccgttcagaacatactcagatgtggcagccgctgtatcccattgggatcacatgtacatcggaatggcagggaaacgtcccttctacaaaatcttggcttttttgggttcttctaatctaaaggccactccagcggtattggcagatcaaggtcaaccagagtatcacactcactgcgaaggcagggcttatttgccacataggatggggaagacccctcccatgctcaatgtaccagagcacttcagaagaccattcaatataggtctttacaagggaacgattgagctcacaatgaccatctacgatgatgagtcactggaagcagctcctatgatctgggatcatttcaattcttccaaattttctgatttcagagagaaggccttaatgtttggcctgattgtcgagaaaaaggcatctggagcgtgggtcctggattctatcagccacttcaaatgagctagtctaacttctagcttctgaacaatccccggtttactcagtctctcctaattccagcctctcgaacaactaatatcctgtcttttctatccctatgaaaaaaactaacagagatcgatctgtttccttgacactatgaagtgccttttgtacttagcctttttattcattggggtgaattgcaagttcaccatagtttttccacacaaccaaaaaggaaactggaaaaatgttccttctaattaccattattgcccgtcaagctcagatttaaattggcataatgacttaataggcacagccatacaagtcaaaatgcccaagagtcacaaggctattcaagcagacggttggatgtgtcatgcttccaaatgggtcactacttgtgatttccgctggtatggaccgaagtatataacacagtccatccgatccttcactccatctgtagaacaatgcaaggaaagcattgaacaaacgaaacaaggaacttggctgaatccaggcttccctcctcaaagttgtggatatgcaactgtgacggatgccgaagcagtgattgtccaggtgactcctcaccatgtgctggttgatgaatacacaggagaatgggttgattcacagttcatcaacggaaaatgcagcaattacatatgccccactgtccataactctacaacctggcattctgactataaggtcaaagggctatgtgattctaacctcatttccatggacatcaccttcttctcagaggacggagagctatcatccctgggaaaggagggcacagggttcagaagtaactactttgcttatgaaactggaggcaaggcctgcaaaatgcaatactgcaagcattggggagtcagactcccatcaggtgtctggttcgagatggctgataaggatctctttgctgcagccagattccctgaatgcccagaagggtcaagtatctctgctccatctcagacctcagtggatgtaagtctaattcaggacgttgagaggatcttggattattccctctgccaagaaacctggagcaaaatcagagcgggtcttccaatctctccagtggatctcagctatcttgctcctaaaaacccaggaaccggtcctgctttcaccataatcaatggtaccctaaaatactttgagaccagatacatcagagtcgatattgctgctccaatcctctcaagaatggtcggaatgatcagtggaactaccacagaaagggaactgtgggatgactgggcaccatatgaagacgtggaaattggacccaatggagttctgaggaccagttcaggatataagtttcctttatacatgattggacatggtatgttggactccgatcttcatcttagctcaaaggctcaggtgttcgaacatcctcacattcaagacgctgcttcgcaacttcctgatgatgagagtttattttttggtgatactgggctatccaaaaatccaatcgagcttgtagaaggttggttcagtagttggaaaagctctattgcctcttttttctttatcatagggttaatcattggactattcttggttctccgagttggtatccatctttgcattaaattaaagcacaccaagaaaagacagatttatacagacatagagatgaaccgacttggaaagtaactcaaatcctgcacaacagattcttcatgtttggaccaaatcaacttgtgataccatgctcaaagaggcctcaattatatttgagtttttaatttttatgaaaaaaactaacagcaatcatggaagtccacgattttgagaccgacgagttcaatgatttcaatgaagatgactatgccacaagagaattcctgaatcccgatgagcgcatgacgtacttgaatcatgctgattacaatttgaattctcctctaattagtgatgatattgacaatttgatcaggaaattcaattctcttccgattccctcgatgtgggatagtaagaactgggatggagttcttgagatgttaacatcatgtcaagccaatcccatctcaacatctcagatgcataaatggatgggaagttggttaatgtctgataatcatgatgccagtcaagggtatagttttttacatgaagtggacaaagaggcagaaataacatttgacgtggtggagaccttcatccgcggctggggcaacaaaccaattgaatacatcaaaaaggaaagatggactgactcattcaaaattctcgcttatttgtgtcaaaagtttttggacttacacaagttgacattaatcttaaatgctgtctctgaggtggaattgctcaacttggcgaggactttcaaaggcaaagtcagaagaagttctcatggaacgaacatatgcaggattagggttcccagcttgggtcctacttttatttcagaaggatgggcttacttcaagaaacttgatattctaatggaccgaaactttctgttaatggtcaaagatgtgattatagggaggatgcaaacggtgctatccatggtatgtagaatagacaacctgttctcagagcaagacatcttctcccttctaaatatctacagaattggagataaaattgtggagaggcagggaaatttttcttatgacttgattaaaatggtggaaccgatatgcaacttgaagctgatgaaattagcaagagaatcaaggcctttagtcccacaattccctcattttgaaaatcatatcaagacttctgttgatgaaggggcaaaaattgaccgaggtataagattcctccatgatcagataatgagtgtgaaaacagtggatctcacactggtgatttatggatcgttcagacattggggtcatccttttatagattattacactggactagaaaaattacattcccaagtaaccatgaagaaagatattgatgtgtcatatgcaaaagcacttgcaagtgatttagctcggattgttctatttcaacagttcaatgatcataaaaagtggttcgtgaatggagacttgctccctcatgatcatccctttaaaagtcatgttaaagaaaatacatggcccacagctgctcaagttcaagattttggagataaatggcatgaacttccgctgattaaatgttttgaaatacccgacttactagacccatcgataatatactctgacaaaagtcattcaatgaataggtcagaggtgttgaaacatgtccgaatgaatccgaacactcctatccctagtaaaaaggtgttgcagactatgttggacacaaaggctaccaattggaaagaatttcttaaagagattgatgagaagggcttagatgatgatgatctaattattggtcttaaaggaaaggagagggaactgaagttggcaggtagatttttctccctaatgtcttggaaattgcgagaatactttgtaattaccgaatatttgataaagactcatttcgtccctatgtttaaaggcctgacaatggcggacgatctaactgcagtcattaaaaagatgttagattcctcatccggccaaggattgaagtcatatgaggcaatttgcatagccaatcacattgattacgaaaaatggaataaccaccaaaggaagttatcaaacggcccagtgttccgagttatgggccagttcttaggttatccatccttaatcgagagaactcatgaattttttgagaaaagtcttatatactacaatggaagaccagacttgatgcgtgttcacaacaacacactgatcaattcaacctcccaacgagtttgttggcaaggacaagagggtggactggaaggtctacggcaaaaaggatggactatcctcaatctactggttattcaaagagaggctaaaatcagaaacactgctgtcaaagtcttggcacaaggtgataatcaagttatttgcacacagtataaaacgaagaaatcgagaaacgttgtagaattacagggtgctctcaatcaaatggtttctaataatgagaaaattatgactgcaatcaaaatagggacagggaagttaggacttttgataaatgacgatgagactatgcaatctgcagattacttgaattatggaaaaataccgattttccgtggagtgattagagggttagagaccaagagatggtcacgagtgacttgtgtcaccaatgaccaaatacccacttgtgctaatataatgagctcagtttccacaaatgctctcaccgtagctcattttgctgagaacccaatcaatgccatgatacagtacaattattttgggacatttgctagactcttgttgatgatgcatgatcctgctcttcgtcaatcattgtatgaagttcaagataagataccgggcttgcacagttctactttcaaatacgccatgttgtatttggacccttccattggaggagtgtcgggcatgtctttgtccaggtttttgattagagccttcccagatcccgtaacagaaagtctctcattctggagattcatccatgtacatgctcgaagtgagcatctgaaggagatgagtgcagtatttggaaaccccgagatagccaagtttcgaataactcacatagacaagctagtagaagatccaacctctctgaacatcgctatgggaatgagtccagcgaacttgttaaagactgaggttaaaaaatgcttaatcgaatcaagacaaaccatcaggaaccaggtgattaaggatgcaaccatatatttgtatcatgaagaggatcggctcagaagtttcttatggtcaataaatcctctgttccctagatttttaagtgaattcaaatcaggcacttttttgggagtcgcagacgggctcatcagtctatttcaaaattctcgtactattcggaactcctttaagaaaaagtatcatagggaattggatgatttgattgtgaggagtgaggtatcctctttgacacatttagggaaacttcatttgagaaggggatcatgtaaaatgtggacatgttcagctactcatgctgacacattaagatacaaatcctggggccgtacagttattgggacaactgtaccccatccattagaaatgttgggtccacaacatcgaaaagagactccttgtgcaccatgtaacacatcagggttcaattatgtttctgtgcattgtccagacgggatccatgacgtctttagttcacggggaccattgcctgcttatctagggtctaaaacatctgaatctacatctattttgcagccttgggaaagggaaagcaaagtcccactgattaaaagagctacacgtcttagagatgctatctcttggtttgttgaacccgactctaaactagcaatgactatactttctaacatccactctttaacaggcgaagaatggaccaaaaggcagcatgggttcaaaagaacagggtctgcccttcataggttttcgacatctcggatgagccatggtgggttcgcatctcagagcactgcagcattgaccaggttgatggcaactacagacaccatgagggatctgggagatcagaatttcgactttttattccaagcaacgttgctctatgctcaaattaccaccactgttgcaagagacggatggatcaccagttgtacagatcattatcatattgcctgtaagtcctgtttgagacccatagaagagatcaccctggactcaagtatggactacacgcccccagatgtatcccatgtgctgaagacatggaggaatggggaaggttcgtggggacaagagataaaacagatctatcctttagaagggaattggaagaatttagcacctgctgagcaatcctatcaagtcggcagatgtataggttttctatatggagacttggcgtatagaaaatctactcatgccgaggacagttctctatttcctctatctatacaaggtcgtattagaggtcgaggtttcttaaaagggttgctagacggattaatgagagcaagttgctgccaagtaatacaccggagaagtctggctcatttgaagaggccggccaacgcagtgtacggaggtttgatttacttgattgataaattgagtgtatcacctccattcctttctcttactagatcaggacctattagagacgaattagaaacgattccccacaagatcccaacctcctatccgacaagcaaccgtgatatgggggtgattgtcagaaattacttcaaataccaatgccgtctaattgaaaagggaaaatacagatcacattattcacaattatggttattctcagatgtcttatccatagacttcattggaccattctctatttccaccaccctcttgcaaatcctatacaagccatttttatctgggaaagataagaatgagttgagagagctggcaaatctttcttcattgctaagatcaggagaggggtgggaagacatacatgtgaaattcttcaccaaggacatattattgtgtccagaggaaatcagacatgcttgcaagttcgggattgctaaggataataataaagacatgagctatcccccttggggaagggaatccagagggacaattacaacaatccctgtttattatacgaccaccccttacccaaagatgctagagatgcctccaagaatccaaaatcccctgctgtccggaatcaggttgggccaattaccaactggcgctcattataaaattcggagtatattacatggaatgggaatccattacagggacttcttgagttgtggagacggctccggagggatgactgctgcattactacgagaaaatgtgcatagcagaggaatattcaatagtctgttagaattatcagggtcagtcatgcgaggcgcctctcctgagccccccagtgccctagaaactttaggaggagataaatcgagatgtgtaaatggtgaaacatgttgggaatatccatctgacttatgtgacccaaggacttgggactatttcctccgactcaaagcaggcttggggcttcaaattgatttaattgtaatggatatggaagttcgggattcttctactagcctgaaaattgagacgaatgttagaaattatgtgcaccggattttggatgagcaaggagttttaatctacaagacttatggaacatatatttgtgagagcgaaaagaatgcagtaacaatccttggtcccatgttcaagacggtcgacttagttcaaacagaatttagtagttctcaaacgtctgaagtatatatggtatgtaaaggtttgaagaaattaatcgatgaacccaatcccgattggtcttccatcaatgaatcctggaaaaacctgtacgcattccagtcatcagaacaggaatttgccagagcaaagaaggttagtacatactttaccttgacaggtattccctcccaattcattcctgatccttttgtaaacattgagactatgctacaaatattcggagtacccacgggtgtgtctcatgcggctgccttaaaatcatctgatagacctgcagatttattgaccattagccttttttatatggcgattatatcgtattataacatcaatcatatcagagtaggaccgatacctccgaaccccccatcagatggaattgcacaaaatgtggggatcgctataactggtataagcttttggctgagtttgatggagaaagacattccactatatcaacagtgtttagcagttatccagcaatcattcccgattaggtgggaggctgtttcagtaaaaggaggatacaagcagaagtggagtactagaggtgatgggctcccaaaagatacccgaacttcagactccttggccccaatcgggaactggatcagatctctggaattggtccgaaaccaagttcgtctaaatccattcaatgagatcttgttcaatcagctatgtcgtacagtggataatcatttgaaatggtcaaatttgcgaagaaacacaggaatgattgaatggatcaatagacgaatttcaaaagaagaccggtctatactgatgttgaagagtgacctacacgaggaaaactcttggagagattaaaaaatcatgaggagactccaaactttaagtatgaaaaaaactttgatccttaagaccctcttgtggtttttattttttatctggttttgtggtcttcgt

SEQ ID NO: 163 - RNA, 15894 nt, Measles virus
  source 1..15894: mol_type = genomic RNA
accaaacaaagttgggtaaggatagatcaatcaatgatcatattctagtacacttaggattcaagatcctattatcagggacaagagcaggattagggatatccgagatggccacacttttgaggagcttagcattgttcaaaagaaacaaggacaaaccacccattacatcaggatccggtggagccatcagaggaatcaaacacattattatagtaccaattcctggagattcctcaattaccactcgatccagactactggaccggttggtcaggttaattggaaacccggatgtgagcgggcccaaactaacaggggcactaataggtatattatccttatttgtggagtctccaggtcaattgattcagaggatcaccgatgaccctgacgttagcatcaggctgttagaggttgttcagagtgaccagtcacaatctggccttaccttcgcatcaagaggtaccaacatggaggatgaggcggaccaatacttttcacatgatgatccaagcagtagtgatcaatccaggtccggatggttcgagaacaaggaaatctcagatattgaagtgcaagaccctgagggattcaacatgattctgggtaccattctagcccagatctgggtcttgctcgcaaaggcggttacggccccagacacggcagctgattcggagctaagaaggtggataaagtacacccaacaaagaagggtagttggtgaatttagattggagagaaaatggttggatgtggtgaggaacaggattgccgaggacctctctttacgccgattcatggtggctctaatcctggatatcaagaggacacccgggaacaaacctaggattgctgaaatgatatgtgacattgatacatatatcgtagaggcaggattagccagttttatcctgactattaagtttgggatagaaactatgtatcctgctcttggactgcatgaatttgctggtgagttatccacacttgagtccttgatgaatctttaccagcaaatgggagaaactgcaccctacatggtaatcctagagaactcaattcagaacaagttcagtgcaggatcataccctctgctctggagctatgccatgggagtaggagtggaacttgaaaactccatgggaggtttgaactttggtcgatcttactttgatccagcatattttagattagggcaagagatggtgaggaggtcagctggaaaggtcagttccacattggcatccgaactcggtatcactgccgaggatgcaaggcttgtttcagagattgcaatgcatactactgaggacaggatcagtagagcggtcggacccagacaagcccaagtgtcatttctacacggtgatcaaagtgagaatgagctaccaggattggggggcaaggaagataggagggtcaaacagggtcggggagaagccagggagagctacagagaaaccgggtccagcagagcaagtgatgcgagagctgcccatcctccaaccagcatgcccctagacattgacactgcatcggagtcaggccaagatccgcaggacagtcgaaggtcagctgacgccctgctcaggctgcaagccatggcaggaatcttggaagaacaaggctcagacacggacacccctagggtatacaatgacagagatcttctagactaggtgcgagaggccgaggaccagaacaacatccgcctaccctccatcattgttataaaaaacttaggaaccaggtccacacagccgccagccaaccaaccatccactcccacgactggagccgatggcagaagagcaggcacgccatgtcaaaaacggactggaatgcatccgggctctcaaggccgagcccatcggctcactggccgtcgaggaagccatggcagcatggtcagaaatatcagacaacccaggacaggaccgagccacctgcaaggaagaggaggcaggcagttcgggtctcagcaaaccatgcctctcagcaattggatcaactgaaggcggtgcacctcgcatccgcggtcagggatctggagaaagcgatgacgacgctgaaactttgggaatcccctcaagaaatctccaggcatcaagcactgggttacagtgttatcatgtttatgatcacagcggtgaagcggttaagggaatccaagatgctgactctatcatggttcaatcaggccttgatggtgatagcaccctctcaggaggagacgatgaatctgaaaacagcgatgtggatattggcgaacctgataccgagggatatgctatcactgaccggggatctgctcccatctctatggggttcagggcttctgatgttgaaactgcagaaggaggggagatccacgagctcctgaaactccaatccagaggcaacaactttccgaagcttgggaaaactctcaatgttcctccgcccccgaaccccagtagggccagcacttccgagacacccattaaaaagggcacagacgcgagattggcctcatttggaacggagatcgcgtctttattgacaggtggtgcaacccaatgtgctcgaaagtcaccctcggaaccatcagggccaggtgcacctgcggggaatgtccccgagtgtgtgagcaatgccgcactgatacaggagtggacacccgaatctggtaccacaatctccccgagatcccagaataatgaagaagggggagactattatgatgatgagctgttctccgatgtccaagacatcaaaacagccttggccaaaatacacgaggataatcagaagataatctccaagctagaatcattgctgttattgaagggagaagttgagtcaattaagaagcagatcaacaggcaaaatatcagcatatccaccctggaaggacacctctcaagcatcatgattgccattcctggacttgggaaggatcccaacgaccccactgcagatgtcgaactcaatcccgacctgaaacccatcataggcagagattcaggccgagcactggccgaagttctcaagaagcccgttgccagccgacaactccagggaatgactaatggacggaccagttccagaggacagctgctgaaggaatttcaactaaagccgatcgggaaaaaggtgagctcagccgtcgggtttgttcctgacaccggccctgcatcacgcagtgtaatccgctccattataaaatccagccggctagaggaggatcggaagcgttacctgatgactctccttgatgatatcaaaggagccaacgatcttgccaagttccaccagatgctgatgaagataataatgaagtagctacagctcaacttacctgccaaccccatgccagtcgacctaattagtacaacctaaatccattataaaaaacttaggagcaaagtgattgcctcctaagttccacaatgacagagatctacgatttcgacaagtcggcatgggacatcaaagggtcgatcgctccgatacaacctaccacctacagtgatggcaggctggtgccccaggtcagagtcatagatcctggtctaggtgataggaaggatgaatgctttatgtacatgtttctgctgggggttgttgaggacagcgatcccctagggcctccaatcgggcgagcattcgggtccctgcccttaggtgttggtagatccacagcaaaacccgaggaactcctcaaagaggccactgagcttgacatagttgttagacgtacagcagggctcaatgaaaaactggtgttctacaacaacaccccactaaccctcctcacaccttggagaaaggtcctaacaacagggagtgtcttcaatgcaaaccaagtgtgcaatgcggttaatctaataccgctggacaccccgcagaggttccgtgttgtttatatgagcatcacccgtctttcggataacgggtattacaccgttcccagaagaatgctggaattcagatcggtcaatgcagtggccttcaacctgctagtgacccttaggattgacaaggcgattggccctgggaagatcatcgacaatgcagagcaacttcctgaggcaacatttatggtccacatcgggaacttcaggagaaagaagagtgaagtctactctgccgattattgcaaaatgaaaatcgaaaagatgggcctggtttttgcacttggtgggatagggggcaccagtcttcacattagaagcacaggcaaaatgagcaagactctccatgcacaactcgggttcaagaagaccttatgttacccactgatggatatcaatgaagaccttaatcggttactctggaggagcagatgcaagatagtaagaatccaggcagttttgcagccatcagttcctcaagaattccgcatttacgacgacgtgatcataaatgatgaccaaggactattcaaagttctgtagaccgcagtgcccagcaatacccgaaaacgacccccctcataatgacagccagaaggcccggacaaaaaagccccctccagaagactccacggaccaagcgagaggccagccagcagccgacagcaagtgtggacaccaggcggcccaagcacagaacagccccgacacaaggccaccaccagccatcccaatctgcgtcctcctcgtgggacccccgaggaccaaccccgaaggtcgctccgaacacagaccaccaaccgcatccccacagctcccgggaaaggaacccccagcaactggaaggcccctccccccctcccccaacgcaagaaccccacaaccgaaccgcacaagcgaccgaggtgacccaaccgcaggcatccgactccttagacagatcctctccccccggcatactaaacaaaacttagggccaaggaacacacacactcgacagaacccagaccccggcccgcggcaccgcgcccccaccccccgaaaaccagagggagcccccaaccaaacccgccggcccccccggtgcccacaggtaggcacaccaacccccgaccagacccagcacccagccaccgacaatccaagacggggggccccccccaaaaaaaggcccccaggggccgacagccagcatcgcgaggaagcacacccaccccacacacgaccacggcaaccgaaccagagtccagaccaccctgggccaccagctcccagactcggccatcaccccgcaaaaaggaaaggccacaacccgcgcaccccagccccgatccggcgggcagccactcaacccgaaccagcacccaagagcgatccctgggggacccccaaaccgcaaaagacatcagtatcccacagcctctccaagtcccccggtctcctcctcttctcgaagggaccaaaagatcaatccaccacatccgacgacactcaattccccacccccaaaggagacaccgggaatcccagaatcaagactcatccagtgtccatcatgggtctcaaggtgaacgtctctgccatattcatggcagtactgttaactctccaaacacccaccggtcaaatccattggggcaatctctctaagataggggtggtagggataggaagtgcaagctacaaagttatgactcgttccagccatcaatcattggtcataaaattaatgcccaatataactctcctcaataactgcacgagggtagagatcgcagaatacaggagactactgagaacagttttggaaccaattagagatgcacttaatgcaatgacccagaatataagaccggttcagagtgtagcttcaagtaggagacacaagagatttgcgggagttgtcctggcaggtgcggccctaggcgttgccacagctgctcagataacagccggcattgcacttcaccagtccatgctgaactctcaagccatcgacaatctgagagcaagcctggaaactactaatcaggcaattgaggcaatcagacaagcagggcaggagatgatattggctgttcagggtgtccaagactacatcaataatgagctgataccgtctatgaaccaactatcttgtgatttaatcggccagaagctagggctcaaattgctcagatactatacagaaatcctgtcattatttggccccagcttacgggaccccatatctgcggagatatccatccaggctttgagctatgcgcttggaggagatatcaataaggtattagaaaagctcggatacagtggaggtgatttactgggcatcttagagagcagaggaataaaggcccggataactcacgtcgacacagagtcctacttcattgtactcagtatagcctatccgacgctgtccgagattaagggggtgattgtccaccggctagagggggtctcgtacaatataggctctcaagagtggtataccactgtgcccaagtatgttgcaacccaagggtaccttatctcgaattttgatgagtcatcgtgtactttcatgccagaggggactgtgtgcagccaaaatgccttgtacccgatgagtcctctgctccaagaatgcctccgggggtccaccaagtcctgtgctcgtacactcgtatccgggtcttttgggaaccggttcattttatcacaagggaacctaatagccaattgtgcatcaatcctctgcaagtgttacacaacaggaacgatcattaatcaagaccctgacaagatcctaacatacattgctgccgatcactgcccggtggtcgaggtgaacggtgtgaccatccaagtcgggagcaggaggtatccggacgcggtgtacctgcacagaattgacctcggtcctcccatatcattggagaggttggacgtagggacaaatctggggaatgcaattgctaagttggaggatgccaaggaattgttggagtcatcggaccagatattgaggagtatgaaaggtttatcgagcactagcatagtttacatcctgattgcagtgtgtcttggagggttgatagggatccccgctttaatatgttgctgcagggggcgctgtaacaaaaagggagaacaagttggtatgtcaagaccaggcctaaagcctgatcttacagggacatcaaaatcctatgtaaggtcgctctgatcctctacaactcttgaaacacagatttcccacaagtctcctcttcgtcatcaagcaaccaccgcatccagcatcaagcccacctgaaattgtctccggcttccctctggccgaacgatatcggtagttaattaaaacttagggtgcaagatcatccacaatgtcaccacaacgagaccgaataaatgccttctacaaagacaacccacatcctaagggaagtaggatagttattaacagagaacatcttatgattgatagaccttatgttttgctggctgttctattcgtcatgtttctgagcttgatcgggttgctagccattgcaggcattagactccatcgtgcagccatctacaccgcagagatccataagagcctcagcaccaatctagatgtaactaactcgatcgagcatcaggtcaaggacgtgctgacaccactcttcaagatcattggtgatgaagtgggcctgaggacacctcagagattcactgacctagtgaaattcatctctgacaaaattaaattccttaatccggatagggagtacgacttcagagatctcacttggtgtatcaacccgccagagagaatcaaattggattatgatcaatactgtgcagatgtggctgctgaagaactcatgaatgcattggtgaactcaactctactggaggccagggcaaccaatcagttcctagctgtctcaaagggaaactgctcagggcccactacaatcagaggtcaattctcaaacatgtcgctgtccctgttggacttgtatttaagtcgaggttacaatgtgtcatctatagtcactatgacatcccagggaatgtacgggggaacttacctagtgggaaagcctaatctgagcagtaaagggtcagagttgtcacaactgagcatgcaccgagtgtttgaagtaggggttatcagaaatccgggtttgggggctccggtgttccatatgacaaactattttgagcaaccagtcagtaatgatttcagcaactgcatggtggctttgggggagcttaaattcgcagccctctgtcacagggaagattctatcacaattccctatcaggggtcagggaaaggtgtcagcttccagctcgtcaagctaggtgtctggaaatccccaaccgacatgcgatcctgggtccccctatcaacggatgatccagtgatagataggctttacctctcatctcacagaggtgttatcgctgacaatcaagcaaaatgggctgtcccgacaacacggacagatgacaagttgcgaatggagacatgcttccagcaggcgtgtaagggtaaaaaccaagcactctgcgagaatcccgagtgggcaccattgaaggataacaggattccttcatacggggtcttgtctgttaatctgagtctgacagttgagcttaaaatcaaaattgcttcaggattcgggccattgatcacacacggttcagggatggacctatacaaaaccaaccacaacaatgtgtattggctgactatcccgccaatgaagaacctagccttaggtgtaatcaacacattggagtggataccgagattcaaggttagtcccaacctcttcactgttccaatcaaggaagcaggcgaggactgccatgccccaacatacctacctgcggaggtggatggtgatgtcaaactcagttccaatctggtaattctacctggtcaggatctccaatatgttttggcaacctacgatacttccagggttgaacatgctgtggtttattatgtttacagcccaagccgctcattttcttacttttatccttttaggttgcctataaagggggtcccaatcgaattacaagtggaatgcttcacatgggacaaaaaactctggtgccgtcacttctgtgtgcttgcggactcagaatctggtggacatatcactcactctgggatggtgggcatgggagtcagctgcacagtcactcgggaagatggaaccaatcgcagatagggctgccagtgaaccgatcacatgatgtcacccagacatcaggcatacccactagtgtgaaatagacatcagaattaagaaaaacgtagggtccaagtggtttcccgttatggactcgctatctgtcaaccagatcttataccctgaagttcacctagatagcccgatagttaccaataagatagtagctatcctggagtatgctcgagtccctcacgcttacagcctggaggaccctacactgtgtcagaacatcaagcaccgcctaaaaaacggattctccaaccaaatgattataaacaatgtggaagttgggaatgtcatcaagtccaagcttaggagttatccggcccactctcatattccatatccaaattgtaatcaggatttatttaacatagaagacaaagagtcaacaaggaagatccgtgagctcctaaaaaagggaaattcgctgtactccaaagtcagtgataaggttttccaatgcctgagggacactaactcacggcttggcctaggctccgaattgagggaggacatcaaggagaaaattattaacttgggagtttacatgcacagctcccaatggtttgagccctttctgttttggtttacagtcaagactgagatgaggtcagtgattaaatcacaaacccatacttgccataggaggagacacacacctgtattcttcactggtagttcagttgagctgttaatctctcgtgaccttgttgctataatcagtaaggagtctcaacatgtatattacctgacgtttgaactggttttgatgtattgtgatgtcatagaggggaggttaatgacagagaccgctatgaccattgatgctaggtatgcagaacttctaggaagagtcagatacatgtggaaactgatagatggtttcttccctgcactcgggaatccaacttatcaaattgtagccatgctggagccactttcacttgcttacctgcaactgagggatataacagtagaactcagaggtgctttccttaaccactgctttactgaaatacatgatgttcttgaccaaaacgggttttctgatgaaggtacttatcatgagttaattgaagccctagattacattttcataactgatgacatacatctgacaggggagattttctcatttttcagaagtttcggccaccccagacttgaagcagtaacggctgctgaaaatgtcaggaaatacatgaatcagcctaaagtcattgtgtatgagactctgatgaaaggtcatgccatattttgtggaatcataatcaacggctatcgtgacaggcacggaggcagttggccacccctgaccctccccctgcatgctgcagacacaatccggaatgctcaagcttcaggtgaagggttaacacatgagcagtgcgttgataactggaaatcatttgctggagtgagatttggctgttttatgcctcttagcctggacagtgatctgacaatgtacctaaaggacaaggcacttgctgctctccaaagggaatgggattcagtttacccgaaagagttcctgcgttacgatcctcccaagggaaccgggtcacggaggcttgtagatgttttccttaatgattcgagctttgacccatatgatatgataatgtatgtcgtaagtggagcctacctccatgaccctgagttcaacctgtcttacagcctgaaagaaaaggagatcaaggaaacaggtagacttttcgctaaaatgacttacaaaatgagggcatgccaagtgatcgctgaaaatctaatctcaaacgggattggcaagtattttaaggacaatgggatggccaaggatgagcacgatttgactaaggcactccacactctggctgtctcaggagtccccaaagatctcaaagaaagtcacaggggggggccagtcttaaaaacctactcccgaagcccagtccacacaagtaccaggaacgttaaagcagaaaaagggtttgtaggattccctcatgtaattcggcagaatcaagacactgatcatccggagaatatagaaacctacgagacagtcagcgcatttatcacgactgatctcaagaagtactgccttaattggagatatgagaccatcagcttatttgcacagaggctaaatgagatttacggattaccctcattttttcagtggctgcataagaggcttgaaacctctgtcctctatgtaagtgaccctcattgcccccccgaccttgacgcccatgtcccgttatgcaaagtccccaatgaccaaatcttcatcaagtaccctatgggaggtatagaagggtattgtcagaagctgtggaccatcagcaccattccctacttatacctggctgcttatgagagcggggtaaggattgcttcgttagtgcaaggggacaatcagaccatagccgtaacaaaaagggtacccagcacatggccttacaaccttaagaaacgggaagctgctagagtaactagagattactttgtaattcttaggcaaaggctacatgacattggccatcacctcaaggcaaatgagacaattgtttcatcacatttttttgtctattcaaaaggaatatattatgatgggctacttgtgtcccaatcactcaagagcatcgcaagatgtgtattctggtcagagactatagttgatgaaacaagggcagcatgcagtaatattgctacaacaatggctaaaagcatcgagagaggttatgaccgttatcttgcatattccctgaacgtcctaaaagtgatacagcaaattttgatctctcttggcttcacaatcaattcaaccatgacccgagatgtagtcatacccctcctcacaaacaacgatctcttaataaggatggcactgttgcccgctcctattggggggatgaattatctgaatatgagcaggctgtttgtcagaaacatcggtgatccagtaacatcatcaattgctgatctcaagagaatgattctcgcatcactaatgcctgaagagaccctccatcaagtaatgacacaacaaccgggggactcttcattcctagactgggctagcgacccttactcagcaaatcttgtatgcgtccagagcatcactagactcctcaagaacataactgcaaggtttgtcctaatccatagtccaaacccaatgttaaaagggttattccatgatgacagtaaagaagaggacgagagactggcggcattcctcatggacaggcatattatagtacctagggcagctcatgaaatcctggatcatagtgtcacaggggcaagagagtctattgcaggcatgctagataccacaaaaggcctgattcgagccagcatgaggaagggggggttaacctctcgagtgataaccagattgtccaattatgactatgaacaatttagagcagggatggtgctattgacaggaagaaagagaaatgtcctcattgacaaagagtcatgttcagtgcagctggctagagccctaagaagccatatgtgggcaagactagctcgaggacggcctatttacggccttgaggtccctgatgtactagaatctatgcgaggccaccttattcggcgtcatgagacatgtgtcatctgcgagtgtggatcagtcaactacggatggttttttgtcccctcgggttgccaactggatgatattgacaaggaaacatcatccttgagagtcccatatattggttctaccactgatgagagaacagacatgaagctcgccttcgtaagagccccaagtagatccttgcgatctgccgttagaatagcaacagtgtactcatgggcttacggtgatgatgatagctcttggaacgaagcctggttgttggcaaggcaaagggccaatgtgagcctggaggagctaagggtgatcactcccatctcgacttcgactaatttagcgcataggttgagggatcgtagcactcaagtgaaatactcaggtacatcccttgtccgagtggcaaggtataccacaatctccaacgacaatctctcatttgtcatatcagataagaaggttgatactaactttatataccaacaaggaatgcttctagggttgggtgttttagaaacattgtttcgactcgagaaagatactggatcatctaacacggtattacatcttcacgtcgaaacagattgttgcgtgatcccgatgatagatcatcccaggatacccagctcccgcaagctagagctgagggcagagctatgtaccaacccattgatatatgataatgcacctttaattgacagagatgcaacaaggctatacacccagagccataggaggcaccttgtggaatttgttacatggtccacaccccaactatatcacattctagctaagtccacagcactatctatgattgacctggtaacaaaatttgagaaggaccatatgaatgaaatttcagctctcataggggatgacgatatcaatagtttcataactgagtttctgcttatagagccaagattattcaccatctacttgggccagtgtgcagccatcaattgggcatttgatgtacattatcatagaccatcagggaaatatcagatgggtgagctgttgtcttcgttcctttctagaatgagcaaaggagtgtttaaggtgcttgtcaatgctctaagccacccaaagatctacaagaaattctggcattgtggtattatagagcctatccatggtccttcacttgatgctcaaaacttgcacacaactgtgtgcaacatggtttacacatgctatatgacctacctcgacctgttgttgaatgaagagttagaagagttcacatttcttttgtgtgaaagcgatgaggatgtagtaccggacagattcgacaacatccaggcaaaacacttgtgtgttctggcagatttgtactgtcaaccagggacctgcccaccgattcgaggtctaaggccggtagagaaatgtgcagttctaaccgatcatatcaaggcagaggctaggttatctccagcaggatcttcgtggaacataaatccaattattgtagaccattactcatgctctctgacttatctccgtcgaggatctatcaaacagataagattgagagttgatccaggattcatttttgacgccctcgctgaggtaaatgtcagtcagccaaaggtcggcagcaacaacatctcaaatatgagcatcaaggatttcagacctccacacgatgatgttgcaaaattgctcaaagatatcaacacaagcaagcacaatcttcccatttcagggggtagtctcgccaattatgaaatccatgctttccgcagaatcgggttaaactcatctgcttgctacaaagctgttgagatatcaacattaattaggagatgccttgagccaggggaagacggcttgttcttgggtgaggggtcgggttctatgttgatcacttataaggagatactaaaactaaacaagtgcttctataatagtggggtttccgccaattctagatctggtcaaagggaattagcaccctatccctccgaagttggccttgtcgaacacagaatgggagtaggtaatattgtcaaggtgctctttaacgggaggcccgaagtcacgtgggtaggcagtatagattgcttcaatttcatagtcagtaatatccctacctctagtgtggggtttatccattcagatatagagaccttacctaacaaagatactatagagaagctagaggaattggcagccatcttatcgatggctctactccttggcaaaataggatcaatactggtgattaagcttatgcctttcagcggggattttgttcagggatttataagctatgtagggtctcattatagagaagtgaaccttgtctaccctaggtacagcaacttcatatctactgaatcttatttagttatgacagatctcaaagctaaccggctaatgaatcctgaaaagatcaagcagcagataattgaatcatctgtgcggacttcacctggacttataggtcacatcctatccattaagcaactaagctgcatacaagcaattgtgggaggcgcagttagtagaggtgatatcaaccctattctgaaaaaacttacacctatagagcaggtgctgatcagttgcgggttggcaattaacggacctaaactgtgcaaagaattaatccaccatgatgttgcctcagggcaagatggattgcttaactctatactcatcctctacagggagttggcaagattcaaagacaaccaaagaagtcaacaagggatgttccacgcttaccccgtattggtaagtagtaggcaacgagaacttgtatctaggatcactcgcaaattttgggggcatattcttctttactccgggaacagaaagttgataaatcggtttatccagaatctcaagtccggttatctagtactagacttacaccagaatatcttcgttaagaatctatccaagtcagagaaacagattattatgacggggggtttaaaacgtgagtgggtttttaaggtaacagtcaaggagaccaaagaatggtacaagttagtcggatacagcgctctgattaaggattaattggttgaactccggaaccctaatcctgccctaggtagttaggcattatttgcaatatattaaagaaaactttgaaaatacgaagtttctattcccagctttgtctggt

SEQ ID NO: 164 - RNA, 15191 nt, unidentified
  source 1..15191: mol_type = unassigned RNA
acgcgaaaaaatgcgtacaacaaacttgcgtaaaccaaaaaaatggggcaaataagaatttgataagtaccacttaaatttaactcccttggttagagatgggcagcaattcattgagtatgataaaagttagattacaaaatttgtttgacaatgatgaagtagcattgttaaaaataacatgctatactgacaaattaatacatttaactaatgcattggctaaggcagtgatacatacaatcaaattgaatggcattgtatttgtgcatgttattacaagtagtgatatttgccctaataataatattgtagtgaaatccaatttcacaacaatgccagtgttacaaaatggaggttatatatgggaaatgatggaattaacacactgctctcaacctaatggcctaatagatgacaattgtgaaattaaattctccaaaaaactaagtgattcaacaatgaccaattatatgaatcaattatctgaattacttggatttgatcttaatccataaattataataaatatcaactagcaaatcaatgtcactaacaccattagttaatataaaacttgacagaagataaaaatggggcaaataaatcaattcagccgacccaaccatggacacaacacacaatgacaccacaccacaaagactgatgatcacagacatgagaccattgtcacttgagactataataatatcactaaccagagacatcataacacacagatttatatacttgataaatcatgaatgtatagtgagaaaacttgatgaaagacaggccacatttacattcctggtcaactatgaaatgaaactattgcacaaagtgggaagcactaaatacaaaaaatatactgaatacaacacaaaatatggcacttttcctatgccaatatttatcaatcatgatgggttcttagaatgcattggcattaagcctacaaagcacactcccataatatacaagtatgatctcaatccatgaatttcaacacaagagtcacacaatctgaaataacaacttcatgcataaccacactccatagttcaaatggagcctgaaaattatagtaatttaaaattaaggagagacataagatgaaagatggggcaaatacaaaaatggctcttagcaaagtcaagttgaatgatacactcaacaaagatcaacttctgtcatccagcaaatacaccatccaacggagcacaggagatagtattgatactcctaattatgatgtgcagaaacacatcaataagttatgtggcatgttattaatcacagaagatgctaatcataaattcactgggttaataggtatgttatatgctatgtctagattaggaagagaagacaccataaaaatactcagagatgcgggatatcatgtaaaagcaaatggagtggatgtaacaacacatcgtcaagatattaatgggaaagaaatgaaatttgaagtgttaacattgtcaagcttaacaactgaaattcaaatcaacattgagatagaatctagaaaatcctacaaaaaaatgctaaaagaaatgggagaggtagctccagaatacaggcatgactctcctgattgtgggatgataatattatgtatagcggcattagtaataaccaaattagcagcaggggatagatctggtcttacagctgtgattaggagggctaataatgtcctaaaaaatgaaatgaaacgttataaaggcttactacccaaggatatagccaacagcttctatgaagtgtttgaaaaatatcctcactttatagatgtttttgttcattttggtatagcacaatcttctaccagaggtggcagtagagttgaagggatttttgctggattgtttatgaatgcctatggtgcagggcaagtgatgttacggtggggggtcttagcaaaatcagttaaaaatattatgctaggacacgctagtgtgcaagcagaaatggaacaagttgtggaggtttatgaatatgcccaaaaattgggtggagaagcagggttctaccatatattgaacaacccaaaagcatcattattgtctttgactcaatttcctcacttctccagtgtagtattaggcaatgctgctggcctaggcataatgggagaatacagaggtacaccaaggaatcaagatctatatgatgctgcaaaagcatatgctgaacaactcaaagaaaatggtgtgattaactacagtgtattagacttgacagcagaagaactagaggctatcaaacatcagcttaatccaaaagataatgatgtagagctttgagttaataaaaaaaatggggcaaataaaacatcatggaaaagtttgctcctgaattccatggagaagatgcaaacaacagagctaccaaattcctagaatcaataaagggcaaattcacatcacctaaagatcccaagaaaaaagatagtatcatatctgtcaactcaatagatatagaagtaaccaaagaaagccctataacatcaaattcaaccattataaacccaacaaatgagacagatgatactgtagggaacaagcccaattatcaaagaaaacctctagtaagtttcaaagaagaccctacgccaagtgataatcccttttcaaaactatacaaagaaaccatagaaacatttgataacaatgaagaagaatctagctattcatatgaagaaataaatgatcagacaaacgataatataacagcaagattagataggattgatgaaaaattaagtgaaatactaggaatgcttcacacattagtagtagcgagtgcaggacctacatctgctcgggatggtataagagatgccatggttggtttaagagaagacatgatagaaaaaatcagaactgaagcattaatgaccaatgacagactagaagctatggcaagactcaggaatgaggaaagtgaaaagatggcaaaagacacatcagatgaagtgtctctcaatccaacatcagagaaattgaacaacctgttggaagggaatgatagtgacaatgatctatcacttgatgatttctgatcagttaccaatctgtacatcaacacacaacaccaacagaagaccaacaaacaaaccaactcacccatccaaccaaacatctatacgccaatcagccaatccaaaactagccacccggaaaaaatagatactatagttacaaaaaaagatggggcaaatatggaaacatacgtgaacaaacttcacgaaggctccacatacacagctgctgttcaatacaatgtcttagaaaaagacgatgaccctgcatcacttacaatatgggtgcccatgttccaatcatccatgccagcagatttacttataaaagaactagctaatgtcaacatactagtgaaacaaatatccacacccaatggaccttcattaagagtcatgataaactcaagaagtgcagtgctagcacaaatgcccagcaaatttaccatatgtgccaatgtgtccttggatgaaagaagcaagctggcatatgatgtaaccacaccctgtgaaatcaaggcatgtagtctaacatgcctaaaatcaaaaaatatgttaactacagttaaagatctcactatgaaaacactcaacccaacacatgacatcattgctttatgtgaatttgaaaatatagtaacatcaaaaaaagtcataataccaacatacctaagatccatcagtgtcagaaataaagatctgaacacacttgaaaatataacaaccactgaattcaaaaatgccatcacaaatgcaaaaatcatcccttactcaggattactgttagtcatcacagtgactgacaacaaaggagcattcaaatacataaagccacaaagtcaatttatagtagatcttggagcttacctagaaaaagaaagtatatattatgttacaacaaattggaagcacacagctacacgatttgcaatcaaacccatggaagattaacctttttcttctacatcagtgagttgattcatacaaactttctacctacattcttcacttcaccatcataatcaccaaccctctgtggttcaactaatcaaacaaaacccatctggagcctcagatcatcccaagtcattgttcatcagatctagtactcaaataagttaataaaaatatccacatggggcaaataatcattggaggaaatccaactaatcacaatatctgtcaacatagacaagtcaacacgccaggcaaaatcaaccaatggaaaatacatccataacaatagaattctcaagcaaattctggccttactttacactaatacacatgataacaacaataatctctttgctaatcataatctccatcatgattgcaatactgaacaaactctgtgaatataacgtattccataacaaaacctttgagctaccaagagctcgagtcaatacatagcattcaccaatctgatggcacaaaacagtaaccttgcatttgtaagtgaacaaccctcacctctttacaaaaccacatcaacatctcaccatgcaagccatcatccatattataaagtagttaattaaaaataatcataacaatgaactaagatattaagactaacaataacgttggggcaaatgcaaacatgtccaaaaacaaggaccaacgcaccaccaagacactagaaaagacctgggacactctcaatcatctattattcatatcatcgtgcttatacaagttaaatcttaaatctatagcacaaatcacattatccattctggcaatgataatctcaacttcacttataattgcagccatcatattcatagcctcggcaaaccacaaagtcacactaacaactgcaatcatacaagatgcaacaagccagatcaagaacacaaccccaacatacctcacccagaatccccagcttggaatcagcttctccaatctgtctgaaactacatcacaaaccaccaccatactagcttcaacaacaccaagtgtcaagtcaaccctgcaatccacaacagtcaagaccaaaaacacaacaacaaccaaaatacaacccagcaagcccaccacaaaacaacgccaaaacaaaccaccaaacaaacccaataatgattttcactttgaagtgttcaactttgtaccttgcagcatatgcagcaacaatccaacctgctgggctatctgtaaaagaataccaaacaaaaaacctggaaagaaaaccaccaccaagcccacaaaaaaaccaaccatcaagacaaccaaaaaagatctcaaacctcaaaccacaaaaccaaaggaagtacctaccaccaagcccacagaaaagccaaccatcaacaccaccaaaacaaacatcagaactacactgctcaccaacaataccacaggaaatccagaacacacaagtcaaaagggaaccctccactcaacctcctccgatggcaatccaagcccttcacaagtctatacaacatccgagtacctatcacaacctccatctccatccaacacaacaaaccagtagtcattaaaaagcgtattattgcaaaaagccatgaccaaatcaaccagaatcaaaatcaactctggggcaaataacaatggagttgccaatcctcaaaacaaatgcaattaccgcaatccttgctgcagtcacactctgttttgcttccagtcaaaacatcactgaagaattttatcaaacaacatgcagtgcagtcagcaaaggctatcttagtgctctaagaactggttggtatactagtgttataactatagaattaagtaatatcaaggaaaataagtgtaatggaacagacgctaaggtaaaattgataaaacaagaattagataaatataaaagtgctgtaacagaattgcagttgctcatgcaaagcacaccggcaaccaacaatcgagccagaagagaactaccaaggtttatgaattatacactcaacaataccaaaaataccaatgtaacattaagcaagaaaaggaaaagaagatttcttggctttttgttaggtgttggatctgcaatcgccagtggcattgctgtatctaaggtcctgcacctagaaggggaagtgaacaaaatcaaaagtgctctactatccacaaacaaggctgtagtcagcttatcaaatggagttagtgtcttaaccagcaaagtgttagacctcaaaaactatatagataaacagttgttacctattgtgaacaagcaaagctgtagcatatcaaacattgaaactgtgatagagttccaacaaaagaacaacagactactagagattaccagggaatttagtgttaatgcaggtgtaactacacctgtaagcacttatatgttaacaaatagtgaattattatcattaatcaatgatatgcctataacaaatgatcagaaaaagttaatgtccaacaatgttcaaatagttagacagcaaagttactctatcatgtccataataaaggaggaagtcttagcatatgtagtacaattaccactatatggtgtaatagatacaccttgttggaaactgcacacatcccctctatgtacaaccaacacaaaggaagggtccaacatctgtttaacaagaaccgacagaggatggtactgtgacaatgcaggatcagtatctttcttcccactagctgaaacatgtaaagttcaatcgaatcgagtattttgtgacacaatgaacagtttaacattaccaagtgaagtaaatctctgcaacattgacatattcaaccccaaatatgattgcaaaattatgacttcaaaaacagatgtaagcagctccgttatcacatctctaggagccattgtgtcatgctatggcaaaactaaatgtacagcatccaataaaaatcgtggaatcataaagacattttctaacgggtgcgattatgtatcaaataagggggttgacactgtgtctgtaggtaatacattatattatgtaaataagcaagaaggcaaaagtctctatgtaaaaggtgaaccaataataaatttctatgacccattagtgttcccctctgatgaatttgatgcatcaatatctcaagtcaatgagaagattaaccagagcctagcatttattcgtaaatccgatgaattattacataatgtaaatgctggtaaatccaccataaatatcatgataactactataattatagtgattatagtaatattgttatcattaattgccgttggactgctcctatactgcaaggccagaagcacaccagtcacactaagcaaggatcaactgagtggtataaataatattgcatttagtaactaaataaaaatagcacctaatcatgttcttacaatggtttcatatctgctcatagacaacccatctatcattggattttcttaaaatctgaacttcatcgaaactctcatctataaaccatctcacttacattatttaagtagattcctagtttatagttatataaaacaattgaataccagattaacttactatttgtaaaaaatgagaactggggcaaatatgtcacgaaggaatccttgcaaatttgaaattcgaggtcattgcttgaatggtaagaggtgtcattttagtcataattattttgaatggccaccccatgcactgcttgtaagacaaaactttatgttaaacagaatacttaagtctatggataaaagcatcgatactttatcagaaataagtggagctgcagagttggacagaacagaagagtatgccctcggtgtagttggagtgctagagagttatataggatctataaataatataactaaacaatcagcatgtgttgccatgagcaaactcctcactgaactcaacagtgatgacatcaaaaaactgagggacaatgaagagccaaattcacccaagataagagtgtacaatactgtcatatcatatattgaaagcaacaggaaaaacaataaacaaactatccatctgttaaaaagattgccagcagacgtattgaagaaaaccataaaaaccacattggatatccacaagagcataaccatcaataacccaaaagaatcaactgttagtgatataaacgaccatgccaaaaataatgatactacctgacaaatatccttgtagtataaattccatactaataacaagtagttgtagagttactatgtataatcaaaagaacacactatatttcaatcaaaacaaccaaaataaccatatatactcaccgaatcaaccattcaatgaaatccattggacctctcaagacttgattgatgcaattcaaaattttctacaacatctaggtattactgatgatatatacacaatatatatattagtgtcataacactcaatcctaatgcttaccacatcatcaaactattaactcaaacaattcaagccatgggacaaaatggatcccattattaatggaaattctgctaatgtgtatctaaccgatagttatttaaaaggtgttatttctttctcagaatgtaatgctttaggaagttacatattcaatggtccttatctcaaaaatgattataccaacttaattagtagacaaaatccattaatagaacacataaatctaaagaaactaaatataacacagtccttaatgtctaagtatcataaaggtgaaataaaaatagaagaacctacttattttcagtcattacttatgacatacaagagtatgacctcgttagaacagattactaccactaatttacttaaaaagataataagaagagctatagaaattagtgatgtcaaagtctatgctatattgaataaactggggcttaaagaaaaagacaagattaaatccaacaatggacaagatgaagacaactcagttattacaaccataatcaaagatgatatacttttagctgttaaggataatcaatctcatcttaaagcagtcaaaaatcactctacaaaacaaaaagatacaatcaaaacaacactcttgaagaaattaatgtgttcaatgcaacatcctccatcatggttaatacattggtttaatttatacacaaaattaaacaacatattaacacagtatcgatcaagtgaggtaaaaaaccatggttttatattgatagacaatcatactctcaatggattccaatttattttgaatcaatatggttgtatagtttatcataaggaactcaaaagaattactgtgacaacctataatcaattcttgacatggaaaaatattagccttagtagattaaatgtttgtttaattacatggattagtaactgtttgaacacattaaataaaagcttaggcttaagatgcggattcaataatgttatcttgacacaactattcctctatggagattgtatactaaaactattccacaatgaggggttctacataataaaagaggtagagggatttattatgtctctaattttaaatataacagaagaagatcaattcagaaaacggttttataatagtatgctcaacaacatcacagatgctgctaataaagctcagaaaaatctgctatcaagagtatgtcatacattattagataagacagtatccgataatataataaatggcagatggataattctattaagtaagttccttaaattaattaagcttgcaggtgacaataaccttaacaatctgagtgaattatattttttgttcagaatatttggacacccaatggtagatgaaagacaagccatggatgctgttaaagttaattgcaacgagaccaaattttacttgttaagcagtttgagtatgttaagaggtgcctttatatatagaattataaaaggatttgtaaataattacaacagatggcctactttaaggaatgctattgttttacccttaagatggttaacttactataaactaaacacttatccttccttgttggaacttacagaaagagatttgattgttttatcaggactacgtttctatcgtgagtttcggttgcctaaaaaagtggatcttgaaatgatcataaatgataaggctatatcacctcctaaaaatttgatatggactagtttccctagaaattatatgccgtcacacatacaaaattatatagaacatgaaaaattaaaattttccgagagtgataaatcaagaagagtattagagtactatttaagagataacaaattcaatgaatgtgatttatacaactgtgtagttaatcaaagttatcttaacaaccctaatcatgtggtatctttgacaggcaaagaaagagaactcagtgtaggtagaatgtttgcaatgcaaccaggaatgttcagacaagttcaaatattagcagagaaaatgatagctgaaaacattttacaattctttcctgaaagtcttacaagatatggtgatctagaactacagaaaatattagaattgaaagcaggaataagtaacaaatcaaatcgttacaatgataattacaacaattacattagtaagtgctctatcatcacagatctcagcaaattcaatcaagcatttcgatatgaaacatcatgtatttgtagtgatgtactggatgaactgcatggtgtacaatctctatttttctggttacatttagctattcctcatgtcacaataatatgcacatataggcatgcacccccctatataagagatcatattgtagatcttaacaatgtagatgaacaaagtggattatatagatatcatatgggtggtatcgaagggtggtgtcaaaaactatggaccatagaagctatatcactattggatctaatatctctcaaagggaaattctcaattactgctttaattaatggtgacaatcaatcaatagatataagtaaaccagtcagactcatggaaggtcaaactcatgctcaagcagattatttgctagcattaaatagtcttaaattactgtataaagagtatgcaggcataggccacaaattaaaaggaactgagacttatatatcaagagatatgcaatttatgagtaaaacaattcaacataacggtgtatattacccagctagtataaagaaagtcctaagagtgggaccgtggataaacactatacttgatgatttcaaagtgagtctagaatctataggtagtttgacacaagaattagaatatagaggagaaagtctattatgcagtttaatatttagaaatgtatggttatataatcaaattgctttacaactaaaaaatcatgcattatgtaacaataaattatatttggacatattaaaggttctgaaacacttaaaaaccttttttaatcttgataatattgatacagcattaacattgtatatgaatttgcccatgttatttggtggtggtgatcccaacttgttatatcgaagtttctatagaagaactcctgatttcctcacagaggctatagttcactctgtgttcatacttagttattatacaaaccatgatttaaaagataaacttcaagatctgtcagatgatagattgaataagttcttaacatgcataatcacatttgacaaaaaccctaatgctgaattcgtaacattgatgagagatcctcaagctttagggtctgagagacaagctaaaattactagcgaaatcaatagactggcagttactgaggttttgagcacagctccaaacaaaatattctccaaaagtgcacaacactataccactacagagatagatctaaatgatattatgcaaaatatagaacctacatatcctcacgggctaagagttgtttatgaaagtttacccttttataaagcagagaaaatagtaaatcttatatccggtacaaaatctataactaacatactggaaaagacttctgccatagacttaacagatattgatagagccactgagatgatgaggaaaaacataactttgcttataaggatatttccattagattgtaacagagataaaagggaaatattgagtatggaaaacctaagtattactgaattaagcaaatatgttagggaaagatcttggtctttatccaatatagttggtgttacatcacctagtatcatgtatacaatggacatcaaatatacaacaagcactatagctagtggcataatcatagagaaatataatgttaacagtttaacacgtggtgagagaggacccactaaaccatgggttggttcatctacacaagagaaaaaaacaatgccagtttataatagacaagttttaaccaaaaaacagagagatcaaatagatctattagcaaaattggattgggtgtatgcatctatagataacaaggatgaattcatggaagaacttagcataggaattcttgggttaacatatgagaaagccaaaaaattatttccacaatatttaagtgttaactatttgcatcgccttacagtcagtagtagaccatgtgaattccctgcatcaataccagcttatagaactacaaattatcactttgatactagccctattaatcgcatattaacagaaaagtatggtgatgaagatattgatatagtattccaaaactgtataagctttggccttagcttaatgtcagtagtagaacaatttactaatgtatgtcctaacagaattattcttatacctaagcttaatgagatacatttaatgaaacctcccatattcacaggtgatgttgatattcacaagttaaaacaagtgatacaaaaacagcatatgtttttaccagacaaaataagtttgactcaatatgtggaattattcttaagtaataaaacactcaaatctggatctcatgttaattctaatttaatattggcgcataagatatctgactattttcataatacttacattttaagtactaatttagctggacattggattctgattatacaacttatgaaagattctaagggtatttttgaaaaagattggggagagggatatataactgatcatatgttcattaatttgaaagttttcttcaatgcttataagacctatctcttgtgttttcataaaggttacggcagagcaaagctggagtgtgatatgaatacttcagatctcctatgtgtattggaattaatagacagtagttattggaagtctatgtctaaggtatttttagaacaaaaagttatcaaatacattcttagccaggatgcaagtttacatagagtaaaaggatgtcatagcttcaaactatggtttcttaaacgtcttaatgtagcagaattcacagtttgcccttgggttgttaacatagattatcatccaacacatatgaaagcaatattaacttatatagatcttgttagaatgggattgataaatatagataaaatatacattaaaaataaacacaaattcaatgatgaattttatacttctaatctcttttacattaattataacttctcagataatactcatctattaactaaacatataaggattgctaattctgaattagaaaataattacaacaaattatatcatcctacaccagaaaccctagaaaatatactaaccaatccggttaaatgtaatgacaaaaagacactgaatgactattgtataggtaaaaatgttgactcaataatgttaccattgttatctaataagaagcttattaaatcgcctacaatgattagaaccaattacagcaaacaagatttgtataatttatttcctacggttgtgattgataaaattatagatcattcaggtaatacagccaaatctaaccaactttacactactacttctcatcaaatacctttagtgcacaatagcacatcactttattgcatgcttccttggcatcatattaatagattcaattttgtatttagttctacaggttgtaaaattagtatagagtatattttaaaagaccttataattaaagatcctaattgtatagcattcataggtgaaggagcagggaatttattattgcgtacagtagtggaacttcatcccgatataagatatatttacagaagtctgaaggattgcaatgatcatagtttacctattgagtttttaaggctgtacaatggacatatcaacattgattatggtgaaaatttgaccattcctgctacagatgcaaccaacaacattcattggtcttatttacatataaagtttgctgaacctatcagtctttttgtctgtgatgctgaattgcctgtaacagtcaactggagtaaaattataatagagtggagcaagcatgtaagaaaatgcaagtactgttcctcagttaataaatgtacgttaatagtaaaatatcatgctcaagatgatatcgatttcaaattagacaatataactatattaaaaacttatgtatgcttaggcagtaagttaaaggggtctgaagtttacttagtccttacaataggtcctgcaaatgtgttcccagtatttaatgtagtacaaaatgctaaattgatactatcaagaaccaaaaatttcatcatgcctaagaaggctgataaagagtctattgatgcaaatattaaaagtttgataccctttctttgttaccctataacaaaaaaaggaattaatactgcattgtcaaaactaaagagtgttgttagtggagatatactatcatattctatagcaggacgtaatgaagttttcagcaataaacttataaatcataagcatatgaacatcttaaaatggttcaatcatgttttaaatttcagatcaacagaactaaactataatcatttatatatggtagaatctacatatccttatctaagtgaattgttaaacagcttgacaactaatgaacttaaaaaactgattaaaatcacaggtagtttgttatacaactttcataatgaataatgaataaaaatcttatattaaaaattcccatagctacacactaacactgtattcaattatagttatttaaaattaaaaattatataattttttaataacttttagtgaactaatcctaaaattatcattttgatctaggaggaataaatttaaatccaaatctaattggtttatatgtatattaactaaactacgagatattagtttttgacactttttttctcgt

SEQ ID NO: 165 - RNA, 15384 nt, Sendai virus
  source 1..15384: mol_type = genomic RNA
accaaacaagagaagaaacttgtttggaatatataatgaagttagacaggattttagggtcaaagtatccaccctgaagagcaggttccagacccttaactttgctgccaaagtacacgatggctgggttgttgagcaccttcgacacgtttagctctaggaggagtgagagtattaataagtcaggaggaggtgctgttatccccggccagaggagcacagtctcagtgttcgtactaggcccaagtgttactgatgatgcagacaagttatccattgcaaccaccttcttagctcactcattagacacagataagcagcactctcagagaggagggttcctagtctccctacttgccatggcttacagtagtccagaattatacttgacaacaaatggagtaaatgccgatgttaagtacgtgatctacaacatagagaaggaccctaagaggacgaagacggatggatttattgtgaagacaagggacatggaatatgagaggaccacagagtggctgttcggacctatggtcaacaagagtccgcttttccaggggcaacgagatgctgcagaccctgatacgctccttcaaatctatgggtatccggcatgcctaggggcaattattgtccaggtctggattgtgctggtgaaggcaatcacgagtagtgccggcctaaggaaaggatttttcaatagattagaggctttcagacaagatggcactgtgaaaggtgccttagttttcactggagagacagttgaggggataggctcggtcatgcgatcccagcaaagcttggtatctctcatggttgagacccttgtgactatgaacactgcgaggtccgatctcactacactagagaagaacatccagatcgttgggaactacatccgagatgcaggactagcctctttcatgaataccatcaaatatggggtagagacaaagatggcggccttaacgctgtcaaacctgaggcccgacatcaacaagctgagaagcctcatagacacctacctgtcaaaaggccccagggctccttttatttgtatcctaaaggaccctgtccatggtgagtttgcaccaggcaactatccagcactctggagctatgcaatgggagtcgccgttgtacagaataaggcgatgcagcagtacgtcacagggagaacataccttgatatggagatgttcttactaggacaagctgtagcaaaggatgctgagtccaagatcagcagtgctctggaggacgagctaggagtgacagatacggctaaggagaggctcagacatcatttggcaaatctgtccggtggggatggcgcgtaccacaagccaacaggcggcggtgcaatcgaggtagctctagacaatgccgatatagacctggagcctgaagctcacacagatcaggacgccagaggttggggtggggatagcggtgacagatgggctcgctcgatgggtagtggccactttatcacactacatggggcagaacggttggaggaggagaccaatgatgaagatgtctccgacatagagagaagaatagctaggaggcttgcagagagacggcaagaggacgctacaacccacgaagacgaaggccgtaataacggtgttgatcatgacgaagaggatgatgccgcggcagcagctgggatgggaggaatctaagatcacacgaggatttaggatacatgacccgtagtaagaaaaacttagggtgaaagctcatctaccgatccgcccagaccaggccacatccaactccattgactaagcccagcagtccagacaaccacggcttcggctacacttcccgcatggatcaagatgccctcatttctaaagaagattctgaagttgagagggaggcgtcaggaggacgagagtcgctctcggatgttatcggattcctcgacgcagtcctatcaagtgaaccaactgacatcggaggggacagaagctggctccacaataccatcaacaccctccaaaggccaggctctacccacagagtcaaaggtgagggcgagggagaagtctcgacatcgtcgacccaagataatcgatcaggtgaggagagtagagtctctgggggaacaagcgagccagaggcagaagcacatgctagaaacgttgataaacaaaatatacactgggccactgggagaggagctagtacagactctgtacctcaggatctgggcaatggaagagactccggaatccttgaagatcctccaaatgagggaggatatccgagatcaggtgctgaagatgaaaaccgagagatggctgcgaaccctgataagaggggagaagaccaagctgaaggacttccagaagagatacgaagaagtgcacccctacctgatgaaagagaaggtagagcagataataatggaagaggcgtggagcctggcagcccacatagtgcaagagtgactggagtcttggtgatccccagtcctgagctcgaagaggccgtgctacaaaggaacaagagacgacccgccaacagcgggtccagatctctcaccccagtggtcgtgcctagcactcggtctccaccaccggaccatgacaatagtacaaggtcaccaccaaggaaacccccaaccacgcaggatgagcacaccaaccccaggaacacccccgccgtcaggatcaaggatcggagacccccaacagggactcgctccgccccagaccgcccgaccgacggctatccaacccacccaagtccagagaccgatgcaacaaaaaagggcatagaagagaacacatcatctatgaaagagatggctacattgttaacgagtcttggtgtaatccagtctgctcaagaattcgagtcgtcccgagacgcgagttatgtgtttgcaaagcgtgccctaaaatctgcaaactatgcagagatggcatttaatgtatgcggcttgatcctttctgctgagaaatctttcgccaatagagtagacgagaataaacagctgcttaaacagatccaagaaagtgtagagtcatttcgggacatatacaagagattctctgagtatcagaaggaacagaactcactgctgatgtctaacctatctacccttcatattatcacagacagaggtggcaagactgacaatccagattctcccacaaggtccccctctgtttttgcaaaaacaaaagagaacaagaccaaagcaactaggtttgacccgtctatggagaccatgggggatatgaggtataaaccagacctactccgagaggatgaatttagagaagagatccgcaacccggtgtaccaggaaagagataccgaacccagagcatccaatgcatcacgtctgcttccatcaagagagaagcctacaatacactccctcaagctcgtcatagagagcagtcccctaagcagagctgaaaaggcagcatatgtgaaatcattatccaagtgcaagacagaccaagaagttaaggccgtcatggagcttgtagaagaagacatagaatcactgactaattaagaactaggtgaggcatctaatcatcctcagtagtagagcgacctaattcatcattagcatcagccagtagagattaagaaaaacttagggtgaaagaaatttcacctaataagatacaatggccgacatctatagattccccaagttctcatatgaggataacggtactgtagagcctctgcctctgagaactggtccggataagaaggccatccctcatatcaggattgtcaaggtaggagaccctccaaagcatggagtgagatacctagatttgttgctcttaggcttctttgagacgccgaagcaaacggccagcttagggagtgtgtccgacctgacagagcacaccggctactcaatctgtggctccggatcgttacccataggtgtagctaaataccacgggtctgatcaggaactcttaaaggcctgcactgatctcagaattacggtgaggaggactgttcgagcaggggagatgatagtatacatggtggattcgatcggtgctccactcctaccatggtcgggcaggctgagacagggaatgatatttaatgccaacaaggtcgcgcttgccccccagtgcctcccggtggacaaggacataagattcagagtggtgtttgtcaatgggacatccctgggggcgatcactatagccaagatcccaaagactcttgcagaccttgcattgcctaattctatctctgttaacctactagtgacactcaagaccgggatctccacggagcaaaagggggtactcccagtactcgatgatcaaggagagaagaagctcaattttatggtgcatcttgggttgatcagaagaaaagtcgggaagatatactccgttgagtactgcaagagcaagatcgagaggatgcggctgatcttctcacttgggttaattggcggcataagcttccatgtccaggtcactgggaccctatctaagacattcatgggccagctcgcatggaaaagagcggtctgcttcccattgatggacgtgaatcctcatatgaacctggtgatttgggcggcgtctgttgagatcacagacgttgatgcggttttccagccggccatccctcgtgatttccgctactaccctaatgtcgtggcaaagaacatagggaggatcaggaagctgtaaatgtgcatccatcagggacctgcgacaatgcctagagcagacatcacttggcagccggagccatagcattgctctttgtattaagtaagaaaaacttagggataaagtcccttgcgtgtgccctgtcgtagaactttctttttggggaaaatggcgacttatatccagagggtacagtgcatatctgcattattgtcagttgtgctcacaacactggtctcgtgtcaaattcctagagataggctctctaatataggggtcatagtcgacgaagggaaatcactgaagattgccgggtcccacgaatctaggtacatcgtattaagtctggtcccagggatagacctcgaaaatgggtgcggaacagctcaggttatccagtataagagcctactgaaccggttgttaatcccattgagagatgccttagatcttcaggaggctctgataactgtcactaatgacacgatgaccggtgctgatgtcccacagtcaagattcttcggcgccgtgattgggaccatagcacttggagtggcaacatcagcacagataaccgcagggatcgcgctagctgaggcgagggaggccaagagagacatagcactcataaaagaatcaatgacaaagacacacaaatctatagaactgctgcaaaacgctgtgggggaacaaattcttgctctaaagacgctccaggatttcgtgaatgatgagatcaaacccgcgataagcgaattgggttgtgagactgccgctttaagactggggataaaactgacacaacattactccgagctgctgactgcatttggatctaactttggaaccatcggagagaagagcctcacactgcaggcactgtcttcactctactctgctaacatcactgagattatgaccacgatcaggacagggcagtccaacatctatgacgtcatttatacggagcagatcaagggaactgtgattgatgtagatctagagagatacatggttaccctgtctgtaaaaatccccattctttctgaagtcccaggtgtgcttatacacaaagcatcgtctatttcttacaatatagacggggaggagtggtatgtgactgtccccagtcacatactcagtcgtgcctccttcctggggggtgcaaacatagctgattgtgtagagtccagattgacctacatatgccctagggatcctgcacaattgatacctgacagccagcaaaaatgtatcttgggggacacaacaaggtgtcctgtcacaaaggttgtggataacatcatccccaaatttgcctttgtaaatggaggtgtcgttgcgaactgcatagcatccacatgtacctgtgggacaggccgaaggccaatcagtcaggatcgctctaaaggtgtagtattcttaactcatgataactgtggactcataggagtcaatgggatagaattgtatgctaataggaaagggcatgatgccacttggggggtccagaatttgacagtcggtcctgcaatcgctatcagacccgtggacatttctctcaaccttgccgctgctactgacttcctgcaagactctagagctgaacttgagaaggcacgaaaaatcctctctgaagtaggtagatggtacaattcaggggcgactttgattacgatcatagtagtcatgattgtagtattggtggtcattatagtgattgtcattgtgctctacagactcaggagatcaatgctaatgagtaatccagccggtcggatatcaagagacacatatacattagagccgaagataaggcatatgtatactaacggtgggttcgatgctatgactgagaaaagatgaccataactcttgttagatatttggtaaagcaggtatggtatttgctgaggtctgtacataataagaaaaacttagggtgaaagttaggttgctcaccactatagctttcacctcaagtaagtacagatcatggatggtgacaggagcaagcgggactcgtactggtctacctcccctggtggtagcactacaaaattagtgtcagactcagagagatcaggtaaagttgatacctggctactgattctcgcattcacccaatgggctttgtcaattgccacggttatcatttgcatcgtaatcgctgctaggcaagggtatagtatggaaagatattcaatgaccgtggaggcactaaacacaagcaacaaagaagtgaaggagtcactcactagtctaataaggcaagaggttatcactagagccgctaacattcagagttctgtgcaaaccgggatcccagtcttgttgaacaagaacagcagagatgtcatccggctgattgaaaagtcgtgcaatagacaggagctcacgcaactttgcgatagcacaattgcagtccatcatgctgagggaattgccccacttgaaccgcatagcttctggagatgccctgcaggagaaccgtatctaagttcagatcctgaagtctcattgctgcctggcccaagcctgttgtctggttcgacaacgatctctggatgtgttaggctcccttcactctcaattggtgaggcgatctatgcctattcgtcaaatctcattacacaaggctgtgctgacatagggaagtcatatcaggtcctgcagctagggtatatatcactaaattcagatatgtttcctgaccttaaccccgtagtgtcccacacttatgacatcaacgacaaccggaaatcgtgctcggtagtggcaactgggaccaggggttatcagctctgctccatgccgattgtagacgaaagaaccgactactcgagtgatggtatcgaagatttggtccttgacattctggatcttaaagggaggactaaatctcatcggtatagcaacagtgagatagatcttgaccacccgttttctgcattataccctagtgtgggcagcggcattgcaacagagggttcactgatattccttgggtatggtgggctaaccaccccgctacaaggtgatacgaaatgtaggatccaaggatgccaacaggtgtcgcaagacacatgcaatgaagctctgaagattacatggctgggcgggaaacaggtagtcagtgtgctcatccaggttaatgattatctttcagagagaccaagaataagagtcacaaccattccaatcactcagaactacctcggagcagaaggcagattactaaaattaggtgatcaagtatacatctacacgagatcatcaggttggcattcccaactgcagataggggtacttgatgtcagccaccctctgactatcagctggacaccccatgaagccttgtctaggccaggaaacgaagattgcaattggtacaatacttgtccgaaggaatgcatatcaggtgtatataccgacgcttatcccctgtctcctgatgcagctaatgtagccaccgttacgttgtatgccaacacttcacgcgtcaatccaacaatcatgtattccaacaccactaacattataaatatgttaaggataaaagatgtccaattagaggctgcatataccacgacatcatgtatcacacactttggcaaaggctactgtttccacatcatcgagatcaaccaaaagagcttgaacaccctgcaacctatgctctttaagaccagcattccgaagttatgcaaggccgagtcgtgaatttcaccgatcaacaggctagtcggctctgccgacacaggggtcattactggatatcctcaacatctcctagtctctcacattctccacagtattaagaaaaacccagggtgaatgggaagcctgtcattggctatggacgggcaagagtctacccaaaacccttctgacatactctatccagaatgtcacctgaactcccctatagtgaggggaaaaatagcacaattacacgttttgttagatgttaaccagccctacattctgaaagatgacagtataataaatatcacaaagcacaaaatcaggaatgggggtttgtcccttcgtcagattaagatcagatctctaggcaaggctcttcaacgcacaataaaggatctagaccgatacacgtttgaaccgtacccaacctactcccaggagttacttaggctcgatataccagagatatgcgacaagatccgatctgttttcgcggtctcggatcggctgactaaggagctgtctaatgggttccaggatctctggttgaatatctttaagcaactaggtaacatagaagggagagaggggtacgatccattgcaggatattagcaccatcccagagataactgagaggtatagcaggaacaaatggtataggccattcctaacttggttcagtatcaaatatgatatgcggtggatgcagaaaactagaccagggggacctctagatacctctaactcacataaccttctagaatgcaaatcatacactctagtaacatatggagatcttgtcatgatactgaacaagtcgacattgacagggtatatcctaacacctgagctggttctgatgtattgtgatgttgtggaagggaggtggaatatgtctgctgcagggcagttagataaaaggtctactgggataacaagcaaaggagaggagttatgggagttagtggattccctcttttcgagtctcggggaggaaatatacaatgtcatcgcattactcgagcctctatcacttgctctcatacaattaagtgatcctgttatacctctacgtggagcatttatgaggcatgtgttgacagagctacagactgttttgacgagtaaagacgtgtatacggacccggaggcagatgctattgtggagtcgttactagctatatttcatgggacttctattgatgagaaagcagagatcttttcattctttcggacattcggtcatcctagcttggaagctgtcaccgctgccgacaaggtgagagctcatatgtatgcacaaaaggcaataaagcttaagaccctacacgagtgtcatgcagtgttctgcacaatcatcataaatggatatagagagaggcatggcgggcagtggcccccttgcgacttccctgatcacgtgtgcctggaacttagaaatgctcaagggtccaatacggccatctcttacgagtgtgcagtagacaactatacaagtttcataggcttcaagttccggaagtttatagaaccacaactggatgaagatctcacgatatatatgaaggacaaagcattatcgcccaggaaggaagcatgggactctgtgtatccagacagtaacctgtactataaagttccagagtctgaagagaccaggaggctcatcgaagtgttcataaatgatgagaatttcaacccagaagacatcatcgattacgtggagtcaggagactggttaaaagatgagaagttcaacatctcgtatagtttgaaagagaaagagatcaagcaagagggccgtctgttcgcaaagatgacttacaaaatgcgagctgtacaggtcctagcagagacactgttggcgaaaggaataggagagttgttcagcgaaaatggaatggtgaaaggagagatagacttacttaaaagactaactactctctctgtctcaggagttcctaggactgattcagtgtacaataatcctagatcatcagagaagagaaatgaaagcatgaagaagagaaactcaaaagggtactgggacgaaaagaagaggtcgagacatgaatttaaagcaacagattcatccacagacggttatgagacgttaagttgtttcctaacaacagacctcaagaagtactgcttaaattggagatttgagagcactgcacttttcgggcagaggtgcaatgagatatttggcttcaagacctttttcaattggatgcatccagtccttgaaaagtgtacaatatatgttggagacccttactgtccggtcgccgacaggatgcatcgacaactgcaagatcatgcagactctggtattttcatacacaatcctagaggaggaatagaaggttattgccagaaactatggaccttaatctcaatcagtgcgatccacctagcagctgtgagggtaggtgtcagggtctctgcaatggttcaaggcgacaatcaagctatagcagtaacatcaagagtacccgtagcccagacttacaagcagaagaaaaatcatgtctatgaagagatcactcgttatttcggtgctctaagacatgtcatgtttgatataggacatgagctaaaattgaacgagaccatcattagtagtaaaatgtttgtctatagtaaaaggatatattatgatggaaagatcctaccgcaatgtctgaaggccttgactagatgtgtattctggtccgagacattggtcgatgagaacagatctgcttgttccaatatctcaacatccatagcaaaggccatcgagaatgggtattctcctatactaggctactgcattgcgttgtacaagacttgtcagcaagtgtgtatatcattagggatgactataaatccaacaatcagcccgaccgtaagagatcaatactttaagggtaaaaattggctgaggtgtgcagtgttaattccggccaatgttgggggattcaactatatgtctacatctaggtgctttgtcaggaatattggggatccggcagtcgcagccctagctgatctcaagaggttcatcagagcagatcttctagataaacaggtgttgtatagggtaatgaatcaagagcctggtgattctagttttctagattgggcttcagatccttattcatgcaaccttccgcactctcagagtataactacaattataaagaatatcactgctaggtctgtgctgcaggaatctccgaatcctctactgtctggtctttttacagagaccagtggcgaagaggatctcaacctggcctcgtttcttatggaccggaaagtcatcctacctagagtggctcatgagatcttgagtaattccttaacaggggttagggaagcaattgcagggatgcttgatacaaccaagtctctagtgagagcgagtgttaagaggggaggattgtcatatgggatattgaggagacttgtcaattatgatctgttgcagtacgagacactgactaggactctcaggaagccggtgaaagacaacattgagtatgagtatatgtgctcagttgagctggctgtcggtttaaggcagaagatgtggatccacctgacgtatgggagacctatacacgggctagaaacaccagaccctttagagctcttaaggggaacatttattgaaggctcagaggtgtgtaagctttgcaggtctgaaggagcggaccccatctatacatggttttacctcccagacaatatagacctggatacacttacaaacgggagtccagctatacggattccctattttggatcagcaactgatgaaagatcagaagctcaactcggatatgtaaggaatctgagtaaaccagcaaaggcggctatacggatagctatggtgtacacgtgggcttacgggacagatgagatatcgtggatggaagctgccctcatagcccagacaagggctaatctgagcttagaaaatctaaagctgctgactcctgtttctacatcaactaatctctctcataggttaaaagacacggcaacccagatgaagttctccagtgcaacactagtccgtgcaagtcggttcataacaatatcaaatgacaacatggcactcaaagaagcaggggagtcgaaggacactaatctcgtatatcagcagattatgctaactgggctaagcttgtttgagttcaatatgaggtataagaaaggttccttagagaagccactgatattacacctacatcttaacaacgggtgctgtataatggaatctccacaggaagcaaatatccccccaagatccactttggatttggaaatcacacaagaaaacaacaaattgatctatgatcccgatccactcagggatgtagacctagagctatttagcaaggtcagagatgtcgtacatacagttgacatgacctattggtcagacgatgaagttatcagggctactagtatatgtactgcaatgacaatagcggatacaatgtctcaattagatagagacaacttaaaggagatgatagcactggtaaatgatgatgatgtcaacagcctgattactgagtttatggtgattgatgttcctctattttgctcaacattcggaggtatcctagtcaatcagtttgcatactcactctacggcttgaatatcaggggaagggaagaaatatggggacacgtagtccggattctgaaagatacctcccacgcagtcttgaaagtcttatccaatgctctgtcccatcccaagatcttcaaacgattctggaatgcaggtgttgtagagcctgtgtatgggcctaacctttcaaatcaggataagacactactagccctctctgtatgtgagtattctgtggacctattcatgcacgactggcaaggaggtgtaccgcttgaggtctttatctgtgataatgacccagatgtggctgacatgaggaggtcctctttcttggctagacaccttgcatacctgtgcagcttggcagagatatctagggatgggccaagactagagtcaatgaactctcttgagaggctagagacattaaagagttatctggaactcacgttccttgatgaccctgtactgaggtacagtcaattgactggcttagtcatcaaagtattcccgtcaactttgacttacatccggaagtcatctataaaggtattaagaacaagaggaataggagtccccgaagttttagaagattgggaccccgaggcggataatgcactgctagacggcatcgcagcagaaatacaacagaatattcccttaggacatcagactagagcccctttttgggggctgagggtatccaagtcacaggtactgcgtctccgggggtacgaagagatcacaaggggtgaggtaggcagatcaggtgttggtctgacgttaccattcgatgggaggtatctatctcaccagctgaggctctttggtgttaacagtactagctgcttaaaagcacttgaacttacctacttattaagccccttggttgacaaggataaagataggctgtttttaggggaaggagctggggctatgctctcctgttatgacgctactcttggcccatgcatcaactattataactcgggggtatactcttgtgatgtcaatgggcagagagagttaaatatatatcctgctgaggtggcattagtgggaaagaaattaaataatgttaccagtctaggtcagagggttaaggtattgttcaacgggaatcctggctcgacatggatcgggaatgatgaatgtgaggctttgatttggaatgaactacagaataattcgataggcctagtccactgtgacatggaaggaggagatcataaggatgatcaagttgtactgcatgagcattacagtgtgatcaggattgcgtatctggtgggggatagagacgttgtgctgataagtaagattgctcccaggctgggcacagattggaccaggcagcttagcctttacctgaggtactgggatgaggtcaacctggtagtgcttaaaacatctaatcctgcttctacagagatgtatctcctctcaaggcatcccaaatctgacatcatagaagatagcaagacagtgctggccagtcttcaccctctgtcaaaggaagacagcatcaagatagaaaagtggatcttgatagagaaagcaaaggctcacgaatgggttacccgggagttgagagaaggaagctcgtcgtcagggatgcttagaccttaccaccaggcactgcagacgtttggttttgaacccaatttatataaactgagcagggatttcctgtccactatgaacatagctgacacccacaactgcatgacagccttcaacagagttttaaaggatacaatctttgagtgggctagaataaccgagtcagacaaaaggctcaaactaactggtaagtatgacctgtaccctgttagagactcaggcaagttgaagacaatttctagaagacttgtgctgtcttgggtatcattatctatgtctacaagactggtaactgggtctttccccgaccaaaagtttgaggcaagactccaattagggatagtttcactgtcatctcgtgaaatcaggaacctgagggttatcacaaaaactatattagataggtttgagaatactatacatagtataacatatagatttctcaccaaagaagtaaagattttgatgaagattttaggagctgtcaagatgttcggggctaggcagaatgaatacacaaccgtggttgatgatggatcattagatgacattgagccatatgacagcttgtgataagtaatcattatcatgcagggttatttaaactccacggtgtctagaagtcttggacttatccatatggcaatagtaagaaaaacttacaaggagacaagaaaatttgaaagaataaatatctcttaaactcttgtctggt

SEQ ID NO: 166 - RNA, 19 nt, synthetic construct
  source 1..19: mol_type = other RNA
gcatgcgacctctgtttga

SEQ ID NO: 167 - RNA, 19 nt, synthetic construct
  source 1..19: mol_type = other RNA
cgtacgctggagacaaact

SEQ ID NO: 168 - RNA, 720 nt, synthetic construct
  source 1..720: mol_type = other RNA
atggtgagcaagggcgaggaggacaacatggccatcatcaaggagttcatgcggttcaaggtgcacatggagggcagcgtgaacggccacgagttcgagatcgagggcgagggcgagggccggccctacgagggcacccagaccgccaagctgaaggtgaccaagggcggccccctgcccttcgcctgggacatcctgagcccccagttcatgtacggcagcaaggcctacgtgaagcaccccgccgacatccccgactacctgaagctgagcttccccgagggcttcaagtgggagcgggtgatgaacttcgaggacggcggcgtggtgaccgtgacccaggacagcagcctgcaggacggcgagttcatctacaaggtgaagctgcggggcaccaacttccccagcgacggccccgtgatgcagaagaagaccatgggctgggaggccagcagcgagcggatgtaccccgaggacggcgccctgaagggcgagatcaagcagcggctgaagctgaaggacggcggccactacgacgccgaggtgaagaccacctacaaggccaagaagcccgtgcagctgcccggcgcctacaacgtgaacatcaagctggacatcaccagccacaacgaggactacaccatcgtggagcagtacgagcgggccgagggccggcacagcaccggcggcatggacgagctgtacaagagcggcaactga

SEQ ID NO: 169 - DNA, 4319 bp, synthetic construct
  source 1..4319: mol_type = other DNA
gggaataaactagtattcttctggtccccacagactcagagagaacccgccaccatgttcgtgttcctggtgctgctgcctctggtgtccagccagtgtgtgaacctgaccaccagaacacagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgtttaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccaggacgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggaatctgcgccagctaccagacacagacaaacagccctcggagagccagaagcgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagcaggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccagaatgcccaggcactgaacaccctggtcaagcagctgtcctccaacttcggcgccatcagctctgtgctgaacgatatcctgagcagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatatgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctgaagggcgtgaaactgcactacacatgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcagcggccgcggccgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 170 - protein, 664 aa, synthetic construct
GARVQRVVGSAPAASLGISTGDVITAVDGAPINSATAMADALNGHHPGDVISVTWQTKSGGTRTGNVTLAEGPPAEFMVDFGALPPEINSARMYAGPGSASLVAAAQMWDSVASDLFSAASAFQSVVWGLTVGSWIGSSAGLMVAAASPYVAWMSVTAGQAELTAAQVRVAAAAYETAYGLTVPPPVIAENRAELMILIATNLLGQNTPAIAVNEAEYGEMWAQDAAAMFGYAAATATATATLLPFEEAPEMTSAGGLLEQAAAVEEASDTAAANQLMNNVPQALQQLAQPTQGTTPSSKLGGLWKTVSPHRSPISNMVSMANNHMSMTNSGVSMTNTLSSMLKGFAPAAAAQAVQTAAQNGVRAMSSLGSSLGSSGLGGGVAANLGRAASVGSLSVPQAWAAANQAVTPAARALPLTSLTSAAERGPGQMLGGLPVGQMGARAGGGLSGVLRVPPRPVMPHSPAAGDIAPPALSQDRFADFPALPLDPSAMVAQVGPQVVNINTKLGYNNAVGAGTGIVIDPNGVVLTNNHVIAGATDINAFSVGSGQTYGVDVVGYDRTQDVAVLQLRGAGGLPSAAIGGGVAVGEPVVAMGNSGGQGGTPRAVPGRVVALGQTVQASDSLTGAEETLNGLIQFDAAIQPGDAGGPVVNGLGQVVGMNTAAS

SEQ ID NO: 171 - protein, 623 aa, unidentified
MGTVNKPVVGVLMGFGIITGTLRITNPVRASVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSTCLYHPNAPQCLSHMNSGCTFTSPHLAQRVASTVYQNCEHADNYTAYCLGISHMEPSFGLILHDGGTTLKFVDTPESLSGLYVFVVYFNGHVEAVAYTVVSTVDHFVNAIEERGFPPTAGQPPATTKPKEITPVNPGTSPLLRYAAWTGGLAAVVLLCLVIFLICTAKRMRVKAYRVDKSPYNQSMYYAGLPVDDFEDSESTDTEEEFGNAIGGSHGGSSYTVYIDKTR

SEQ ID NO: 172 - protein, 535 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATGSGSAICSGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSRTLPSEVNLCNVDIFNPKYDCKIMTSKTDVSSSVITSLGAIVSCYGKTKCTASNKNRGIIKTFSNGCDYVSNKGVDTVSVGNTLYCVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 173 - protein, 510 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATGSGSAICSGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSRTLPSEVNLCNVDIFNPKYDCKIMTSKTDVSSSVITSLGAIVSCYGKTKCTASNKNRGIIKTFSNGCDYVSNKGVDTVSVGNTLYCVNKQEGKSLYVKGEPIINFYDPLVFPSDEFDASISQVNEKINQSLAFIRKSDELLHNVNAGKSTTNIMITTIIIVIIVILLSLIAVGLLLY

SEQ ID NO: 174 - protein, 529 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATGSGSAICSGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSRTLPSEVNLCNVDIFNPKYDCKIMTSKTDVSSSVITSLGAIVSCYGKTKCTASNKNRGIIKTFSNGCDYVSNKGVDTVSVGNTLYCVNKQEGKSLYVKGEPIINFYDPLVFPSDEFDASISQVNEKINQSLAFIRKSDELLSAIGGYIPEAPRDGQAYVRKDGEWVLLSTFLGGLVPRGSHHHHHHSAWSHPQFEK

SEQ ID NO: 175 - protein, 574 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 176 - protein, 410 aa, synthetic construct
MDPNANPNANPNANPNANPNANPNANPNANPNANPNANPNANPNANPNANPNANPNANPNANPNANPNANPNANPNKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKMEKCSSVPVTNMENITSGFLGPLLVLQAGFFLLTRILTIPQSLDSWWTSLNFLGGSPVCLGQNSQSPTSNHSPTSCPPICPGYRWMCLRRFIIFLFILLLCLIFLLVLLDYQGMLPVCPLIPGSTTTNTGPCKTCTTPAQGNSMFPSCCCTKPTDGNCTCIPIPSSWAFAKYLWEWASVRFSWLSLLVPFVQWFVGLSPTVWLSAIWMMWYWGPSLYSIVSPFIPLLPIFFCLWVYI

SEQ ID NO: 177 - protein, 392 aa, synthetic construct
MMAPDPNANPNANPNANPNANPNANPNANPNANPNANPNANPNANPNANPNANPNANPNANPNANPNANPNANPNANPNKRNVDENANANSAVKNNNNEEPSDKHIKEYLNKIQNSLSTEWSPCSVTCGNGIQVRIKPGSANKPMEKCSSVFNVVNSSIGLGPVTNMENITSGFLGPLLVLQAGFFLLTRILTIPQSLDSWWTSLNFLGGSPVCLGQNSQSPTSNHSPTSCPPICPGYRWMCLRRFIIFLFILLLCLIFLLVLLDYQGMLPVCPLIPGSTTTNTGPCKTCTTPAQGNSMFPSCCCTKPTDGNCTCIPIPSSWAFAKYLWEWASVRFSWLSLLVPFVQWFVGLSPTVWLSAIWMMWYWGPSLYSIVSPFIPLLPIFFCLWVYI

SEQ ID NO: 178 - protein, 226 aa, synthetic construct
MENITSGFLGPLLVLQAGFFLLTRILTIPQSLDSWWTSLNFLGGSPVCLGQNSQSPTSNHSPTSCPPICPGYRWMCLRRFIIFLFILLLCLIFLLVLLDYQGMLPVCPLIPGSTTTNTGPCKTCTTPAQGNSMFPSCCCTKPTDGNCTCIPIPSSWAFAKYLWEWASVRFSWLSLLVPFVQWFVGLSPTVWLSAIWMMWYWGPSLYSIVSPFIPLLPIFFCLWVYI

SEQ ID NO: 179 - protein, 725 aa, synthetic construct
MHHTAASDNFQLSQGGQGFAIPIGQAMAIAGQIRSGGGSPTVHIGPTAFLGLGVVDNNGNGARVQRVVGSAPAASLGISTGDVITAVDGAPINSATAMADALNGHHPGDVISVTWQTKSGGTRTGNVTLAEGPPAEFMVDFGALPPEINSARMYAGPGSASLVAAAQMWDSVASDLFSAASAFQSVVWGLTVGSWIGSSAGLMVAAASPYVAWMSVTAGQAELTAAQVRVAAAAYETAYGLTVPPPVIAENRAELMILIATNLLGQNTPAIAVNEAEYGEMWAQDAAAMFGYAAATATATATLLPFEEAPEMTSAGGLLEQAAAVEEASDTAAANQLMNNVPQALQQLAQPTQGTTPSSKLGGLWKTVSPHRSPISNMVSMANNHMSMTNSGVSMTNTLSSMLKGFAPAAAAQAVQTAAQNGVRAMSSLGSSLGSSGLGGGVAANLGRAASVGSLSVPQAWAAANQAVTPAARALPLTSLTSAAERGPGQMLGGLPVGQMGARAGGGLSGVLRVPPRPYVMPHSPAAGDIAPPALSQDRFADFPALPLDPSAMVAQVGPQVVNINTKLGYNNAVGAGTGIVIDPNGVVLTNNHVIAGATDINAFSVGSGQTYGVDVVGYDRTQDVAVLQLRGAGGLPSAAIGGGVAVGEPVVAMGNSGGQGGTPRAVPGRVVALGQTVQASDSLTGAEETLNGLIQFDAAIQPGDAGGPVVNGLGQVVGMNTAAS

SEQ ID NO: 180 - protein, 228 aa, synthetic construct
MKTIIALSYILCLVFAQKLPGNDNSTATLCLGHHAVPNGTIVKTITNDQIEVTNATELVFPGCGVLKLATGMRNVPEKQTRGIFGAIAGFIENGWEGMVDGWYGFRHQNSEGIGQAADLKSTQAAINQINGMVNRVIELMEQGGPDCYLAELLVALLNQHTIDLTDSEMRKLFERTKKQLRENAEDMGNGCFKIYHKCDNACIGSIRNGTYDHDVYRDEALNNRFQIK

SEQ ID NO: 181 - protein, 388 aa, synthetic construct
MYKIVVIIALLGAVKGLDKICLGHHAVANGTIVKTLTNEQEEVTNATELVFPGCGVLMLATGMRNVPELIQGRGLFGAIAGFLENGWEGMVDGWYGFRHQNAQGTGQAADYKSTQAAIDQITGMVNRVVALMAQGGPDCYLAELLVAMLNQHVIDMADSEMRNLYERVRKQLRQNAEEDGKGCFEIYHACDDSCMESIRNNTYDHSQYREEALLNRLNINSGGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 182 - protein, 215 aa, synthetic construct
MKAKLLVLLCTFTATYADTICIGYHANNSTDTVDTVLEKNVTVTHSVNLGSGLRMVTGLRNIPQRETRGLFGAIAGFIEGGWTGMVDGWYGYHHQNEQGSGYAADQKSTQNAINGITNMVNSVIEKMGSGGSGTDLAELLVLLLNERTLDFHDSNVKNLYEKVKSQLKNNAKEIGNGCFEFYHKCNNECMESVKNGTYDYPKYSEESKLNREKID

SEQ ID NO: 183 - protein, 229 aa, synthetic construct
MNTQILVFALIAIIFAQKLPGNPTNADKICLGHHAVSNGTKVNTLTERGVEVVNATELVFPGCGVLKLATGMKNVPEIPKGRGLFGAIAGFIENGWEGLIDGWYGFRHQNAQGEGTAADYKSTQSAIDQITGMVNRVIELMEQGGPDCYLAELLVAMLNQHVIDLADSEMDKLYERVKRQLRENAEEDGTGCFEIFHKCDDDCMASIRNNTYDHSKYREEAMQNRIQID

SEQ ID NO: 184 - protein, 512 aa, synthetic construct
MKAFSVTCLSFAVFSSSICVNINILQQIGYIKQQVRQLSYYSQSSSSYIVVKLLPNIQPTDDSCEFKSVTQYNKTLSNLLLPIAENINNIASPSPGSRRHGGGAGIAIGIAALGVATAAQVTAAVSLVQAQTNARAIAAMKNSIQATNRAVFEVKEGTQQLAIAVQAIQDHINTIMNTQLNNMSCQILDNQLATSLGLYLTELTTCFQPQLTNPALSPISIQCLRSLLGSMTPAVVQATLSTSISAAEILSAGLMEGQIISVLLDEMQMIVKINIPTIVTQSNALVIDFYSISSFINNQESIIQLPDRILEIGNEQWSYPAKNCKLTRHHIFCQYNEAERLSLESKLCLAGNISACVFSPIAGSYMRRFVALDGTIVANCRSLTCLCKSPSYPIYQPDHHAVTTIDLTACQTLSLDGLDFSIVSLSNITYAENLTISLSQTINTQPIISTELSKVNASLQNAVKYIKESNHQLQSVSVNSKIIEDKIEEILSKIYHIENEIARIKKLIGEAP

SEQ ID NO: 185 - protein, 959 aa, synthetic construct
MYSMQLASCVTLTLVLLVNSQIHWGNLSKIGVVGIGSASYKVMTRSSHQSLVIKLMPNITLLNNCTRVEIAEYRRLLRTVLEPIRDALNAVTQNIRPVQSVASSRRHGGGAGVVLAGAALGVATAAQITAGIALHQSMLNSQAIDNLRASLETTNQAIEAICQAGQECILAVQGVQDYINNELIPSMNQLSCDLIGQKLGLKLLRYYTEILSLFGPSLRDPISAEISIQALSYALGGDINKVLEKLGYSGGDLLGILESRGIKARITHVDTESYFIVLSIAYPTLSEIKGVIVHRLEGVSYNIGSQEWYTTVPKYVATQGYLISNFDESSCTFMPEGTVCSQNALYPMSPLLQECLRGSTKSCARTLVSGSFGNRFILSQGNLIANCASILCKCYTTGTIINQDPDKILTYIAADHCPVVEVNGVTIQVGSRRYPDAVYLHRIDLGPPISLERLDVGTNLGNAIAKLEDAKELLESSDQILSAIEDKIEEILSKIYHIENEIARIKKLIGEAPGSGGGGGGFLAVSKGNCSGPTTIRGQFSNMSLSLLDLYLGRGYNVSSIVTMTSQGMYGGTYLVEKPNLSSKRSELSQLSMYRVFEVGVIRNPGLGAPVFHMTNYLEQPVSNDLSNCMVALGELKLAALCHGEDSITIPYQGSGKGVSFQLVKLGVWKSPADMQSWVPLSTDDPVIDRLYLSSHRGVIADNQAKWAVPTTRTDDKLRMETCFQQACKGKIQTLCENPEWAPLKDNRIPSYGVLSVDLSLTVELKIKIASGFGPLITHGSGMDLYKSNHNNVYWLTIPPMKNLALGVINTLEWIPRFKVSPYLFTVPIKEAGEDCHAPTYLPAEVDGDVKLSSNLVILPGQDLQYVLATYDTSRVEHAVVYYVYSPGRSFSYFYPFRLPIKGVPIELQVECFTWDQKLWCRHFCVLADSESGGHITHSGMVGMGVSCTVTREDGTNRR

SEQ ID NO: 186 - protein, 172 aa, synthetic construct
DTITLPCRPAPPPHCSSNITGLILTRQGGYSNDNTVIFRPSGGDWRDIARCQIAGTVVSTQLFLNGSLAEEEVVIRSEDWRDNAKSICVQLNTSVEINCTGAGHCNISRAKWNNTLKQIASKLREQYGNKTIIFKPSSGGDPEFVNHSFNCGGEFFYCDSTQLFNSTWFNST

SEQ ID NO: 187 - protein, 500 aa, synthetic construct
MWRPSDSTVYVPPPNPVSKVVATDAYVTRTNIFYHASSSRLLAVGHPYFSIKRANKTVVPKVSGYQYRVFKVVLPDPNKFALPDSSLFDPTTQRLVWACTGLEVGRGQPLGVGVSGHPFLNKYDDVENSGSGGNPGQDNRVNVGMDYKQTQLCMVGCAPPLGEHWGKGKQCTNTPVQAGDCPPLELITSVIQDGDMVDTGFGAMNFADLQTNKSDVPIDICGTTCKYPDYLQMAADPYGDRLFFFLRKEQMFARHFFNRAGEVGEPVPDTLIIKGSGNRTSVGSSIYVNTPSGSLVSSEAQLFNKPYWLQKAQGHNNGICWGNQLFVTVVDTTRSTNMTLCASVTTSSTYTNSDYKEYMRHVEEYDLQFIFQLCSITLSAEVMAYIHTMNPSVLEDWNFGLSPPPNGTLEDTYRYVQSQAITCQKPTPEKEKPDPYKNLSFWEVNLKEKFSSELDQYPLGRKFLLQSGYRGRSSIRTGVKRPAVSKASAAPKRKRAKTKR

SEQ ID NO: 188 - protein, 500 aa, synthetic construct; identical to SEQ ID NO: 187

SEQ ID NO: 189 - protein, 501 aa, synthetic construct
MWRPSDSTVYVPPPNPVSKVVATDAYVKRTNIFYHASSSRLLAVGHPYYSIKKVNKTVVPKVSGYQYRVFKVVLPDPNKFALPDSSLFDPTTQRLVWACTGLEVGRGQPLGVGVSGHPLLNKYDDVENSGGYGGNPGQDNRVNVGMDYKQTQLCMVGCAPPLGEHWGKGTQCSNTSVQNGDCPPLELITSVIQDGDMVDTGFGAMNFADLQTNKSDVPLDICGTVCKYPDYLQMAADPYGDRLFFYLRKEQMFARHFFNRAGTVGEPVPDDLLVKGGNNRSSVASSIYVHTPSGSLVSSEAQLFNKPYWLQKAQGHNNGICWGNHLFVTVVDTTRSTNMTLCASVSKSATYTNSDYKEYMRHVEEFDLQFIFQLCSITLSAEVMAYIHTMNPSVLEDWNFGLSPPPNGTLEDTYRYVQSQAITCQKPTPEKEKQDPYKDMSFWEVNLKEKFSSELDQFPLGRKFLLQSGYRGRTSARTGIKRPAVSKPSTAPKRKRTKTKK

SEQ ID NO: 190 - protein, 505 aa, synthetic construct
MSLWLPSEATVYLPPVPVSKVVSTDEYVARTNIYYHAGTSRLLAVGHPYFPIKKPNNNKILVPKVSGLQYRVFRIHLPDPNKFGFPDTSFYNPDTQRLVWACVGVEVGRGQPLGVGISGHPLLNKLDDTENASAYAANAGVDNRECISMDYKQTQLCLIGCKPPIGEHWGKGSPCTNVAVNPGDCPPLELINTVIQDGDMVDTGFGAMDFTTLQANKSEVPLDICTSICKYPDYIKMVSEPYGDSLFFYLRREQMFVRHLFNRAGAVGENVPDDLYIKGSGSTANLASSNYFPTPSGSMVTSDAQIFNKPYWLQRAQGHNNGICWGNQLFVTVVDTTRSTNMSLCAAISTSETTYKNTNFKEYLRHGEEYDLQFIFQLCKITLTADVMTYIHSMNSTILEDWNFGLQPPPGGTLEDTYRFVTSQAIACQKHTPPAPKEDPLKKYTFWEVNLKEKFSADLDQFPLGRKFLLQAGLKAKPKFTLGKRKATPTTSSTSTTAKRKKRKL

SEQ ID NO: 191 - protein, 568 aa, synthetic construct
MCLYTRVLILHYHLLPLYGPLYHPQPLPLHSILVYMVHIIICGHYIILFLRNVNVFPIFLQMALWRPSDNTVYLPPPSVARVVNTDDYVTRTSIFYHAGSSRLLTVGNPYFRVPAGGGNKQDIPKVSAYQYRVFRVQLPDPNKFGLPDNSIYNPETQRLVWACAGVEIGRGQPLGVGLSGHPFYNKLDDTESSHAATSNVSEDVRDNVSVDYKQTQLCILGCAPAIGEHWAKGTACKSRPLSQGDCPPLELKNTVLEDGDMVDTGYGAMDFSTLQDTKCEVPLDICQSICKYPDYLQMSADPYGDSMFFCLRREQLFARHFWNRAGTMGDTVPQSLYIKGTGMRASPGSCVYSPSPSGSIVTSDSQLFNKPYWLHKAQGHNNGVCWHNQLFVTVVDTTRSTNLTICASTQSPVPGQYDATKFKQYSRHVEEYDLQFIFQLCTITLTADVMSYIHSMNSSILEDWNFGVPPPPTTSLVDTYRFVQSVAITCQKDAAPAENKDPYDKLKFWNVDLKEKFSLDLDQYPLGRKFLVQAGLRRKPTIGPRKRSAPSATTSSKPAKRVRVRARK

SEQ ID NO: 192 - protein, 504 aa, synthetic construct
MSLWRPSEATVYLPPVPVSKVVSTDEYVTRTNIYYHAGSARLLTVGHPYYSIPKSDNPKKIVVPKVSGLQYRVFRVRLPDPNKFGFPDTSFYNPETQRLVWACVGLEVGRGQPLGVGISGHPLLNKFDDTENSNRYAGGPGTDNRECISMDYKQTQLCLLGCKPPIGEHWGKGSPCSNNAITPGDCPPLELKNSVIQDGDMVDTGFGAMDFTALQDTKSNVPLDICNSICKYPDYLKMVAEPYGDTLFFYLRREQMFVRHFFNRSGTVGESVPTDLYIKGSGSTATLANSTYFPTPSGSMVTSDAQIFNKPYWMQRAQGHNNGICWGNQLFVTVVDTTRSTNMSVCAAIANSDTTFKSSNFKEYLRHGEEFDLQFIFQLCKITLSADIMTYIHSMNPAILEDWNFGLTTPPSGSLEDTYRFVTSQAITCQKTAPQKPKEDPFKDYVFWEVNLKEKFSADLDQFPLGRKFLLQAGYRARPKFKAGKRSAPSASTTTPAKRKKTKK

SEQ ID NO: 193 - protein, 499 aa, synthetic construct
MSVWRPSEATVYLPPVPVSKVVSTDEYVSRTSIYYYAGSSRLLAVGHPYFSIKNPTNAKKLLVPKVSGLQYRVFRVRLPDPNKFGFPDTSFYNPDTQRLVWACVGLEIGRGQPLGVGISGHPLLNKFDDTETGNKYPGQPGADNRECLSMDYKQTQLCLLGCKPPTGEHWGKGVACTNAAPANDCPPLELINTIIEDGDMVDTGFGCMDFKTLQANKSDVPIDICGSTCKYPDYLKMTSEPYGDSLFFFLRREQMFVRHFFNRAGTLGEAVPDDLYIKGSGTTASIQSSAFFPTPSGSMVTSESQLFNKPYWLQRAQGHNNGICWGNQVFVTVVDTTRSTNMTLCTQVTSDSTYKNENFKEYIRHVEEYDLQFVFQLCKVTLTAEVMTYIHAMNPDILEDWQFGLTPPPSASLQDTYRFVTSQAITCQKTVPPKEKEDPLGKYTFWEVDLKEKFSADLDQFPLGRKFLLQAGLKAKPKLKRAAPTSTRTSSAKRKKVKK

SEQ ID NO: 194 - protein, 539 aa, synthetic construct
MAHNIIYGHGIIIFLKNVNVFPIFLQMALWRPSDSTVYLPPPSVARVVSTDDYVSRTSIFYHAGSSRLLTVGNPYFRVVPNGAGNKQAVPKVSAYQYRVFRVALPDPNKFGLPDSTIYNPETQRLVWACVGMEIGRGQPLGIGLSGHPFYNKLDDTESAHAATAVITQDVRDNVSVDYKQTQLCILGCVPAIGEHWAKGTLCKPAQLQPGDCPPLELKNTIIEDGDMVDTGYGAMDFSTLQDTKCEVPLDICQSICKYPDYLQMSADPYGDSMFFCLRREQLFARHFWNRAGVMGDTVPTDLYIKGTSANMRETPGSCVYSPSPSGSIITSDSQLFNKPYWLHKAQGHNNGICWHNQLFVTVVDTTRSTNLTLCASTQNPVPSTYDPTKFKQYSRHVEEYDLQFIFQLCTITLTAEVMSYIHSMNSSILENWNFGVPPPPTTSLVDTYRFVQSVAVTCQKDTTPPEKQDPYDKLKFWTVDLKEKFSSDLDQYPLGRKFLVQAGLRRRPTIGPRKRPAASTSTASTASRPAKRVRIRSKK

SEQ ID NO: 195 - protein, 529 aa, synthetic construct
MVQILFYILVIFYYVAGVNVFHIFLQMSVWRPSEATVYLPPVPVSKVVSTDEYVSRTSIYYYAGSSRLLTVGHPYFSIKNTSSGNGKKVLVPKVSGLQYRVFRIKLPDPNKFGFPDTSFYNPETQRLVWACTGLEIGRGQPLGVGISGHPLLNKFDDTETSNKYAGKPGIDNRECLSMDYKQTQLCILGCKPPIGEHWGKGTPCNNNSGNPGDCPPLQLINSVIQDGDMVDTGFGCMDFNTLQASKSDVPIDICSSVCKYPDYLQMASEPYGDSLFFFLRREQMFVRHFFNRAGTLGDPVPGDLYIQGSNSGNTATVQSSAFFPTPSGSMVTSESQLFNKPYWLQRAQGHNNGICWGNQLFVTVVDTTRSTNMTLCAEVKKESTYKNENFKEYLRHGEEFDLQFIFQLCKITLTADVMTYIHKMDATILEDWQFGLTPPPSASLEDTYRFVTSTAITCQKNTPPKGKEDPLKDYMFWEVDLKEKFSADLDQFPLGRKFLLQAGLQARPKLKRPASSAPRTSTKKKKVKR

SEQ ID NO: 196 - protein, 524 aa, synthetic construct
MVLILCCTLAILFCVADVNVFHIFLQMSVWRPSEATVYLPPVPVSKVVSTDEYVSRTSIYYYAGSSRLLAVGNPYFSIKSPNNNKKVLVPKVSGLQYRVFRVRLPDPNKFGFPDTSFYNPDTQRLVWACVGLEIGRGQPLGVGVSGHPYLNKFDDTETSNRYPAQPGSDNRECLSMDYKQTQLCLIGCKPPTGEHWGKGVACNNNAAATDCPPLELFNSIIEDGDMVDTGFGCMDFGTLQANKSDVPIDICNSTCKYPDYLKMASEPYGDSLFFFLRREQMFVRHFFNRAGKLGEAVPDDLYIKGSGNTAVIQSSAFFPTPSGSIVTSESQLFNKPYWLQRAQGHNNGICWGNQLFVTVVDTTRSTNMTLCTEVTKEGTYKNDNFKEYVRHVEEYDLQFVFQLCKITLTAEIMTYIHTMDSNILEDWQFGLTPPPSASLQDTYRFVTSQAITCQKTAPPKEKEDPLNKYTFWEVNLKEKFSADLDQFPLGRKFLLQSGLKAKPRLKRSAPTTRAPSTKRKKVKK

(19) United States
(12) Patent Application Publication
(10) Pub. No.: US 2026/0007741 A1
(43) Pub. Date: Jan. 8, 2026

(54) LIPID NANOPARTICLES FOR DELIVERY OF NUCLEIC ACIDS AND VACCINES

(71) Applicant: Akagera Medicines, Inc., Boxford, MA (US)

(72) Inventors: Daryl C. Drummond, Lincoln, MA (US); Ross Fulton, Southborough, MA (US); Mark E. Hayes, Mill Valley, CA (US); Dmitri Kirpotin, San Francisco, CA (US); Alexander Koshkaryev, Norwood, MA (US)

(73) Assignee: Akagera Medicines, Inc., Boxford, MA (US)

(21) Appl. No.: 19/243,498

(22) Filed: Jun. 19, 2025

Related U.S. Application Data
(60) Provisional application No. 63/662,926, filed on Jun. 21, 2024.

(51) Int. Cl. A61K 39/39; A61K 9/51; A61K 39/00

(52) U.S. Cl. CPC A61K 39/39; A61K 9/5146; A61K 2039/55555; A61K 2039/55561

(57) ABSTRACT

Aspects of the present disclosure provides for improved vaccine compositions of ionizable lipid nanoparticles for the delivery of immunogenic nucleic acids to cells. Anionic phospholipids, including phosphatidylserine and phosphatidylglycerol are included in the lipid nanoparticles to increase the transfection efficiency in dendritic cells. In some embodiments, the incorporation of mono-unsaturated alkyl chain analogs in dimethylaminopropyl-dioxolane or heterocyclic ketal ionizable lipids in the formulation provided high levels of transfection in human dendritic cells, compared to other ionizable lipids in the same family, and demonstrated good stability to oxidative damage.

### RELATED APPLICATIONS

This patent application claims the benefit of and priority to U.S. Provisional Patent Application No. 63/662,926, filed on Jun. 21, 2024, which is incorporated herein by reference in its entirety.

### SEQUENCE LISTING

The instant application contains a Sequence Listing which has been submitted electronically in XML format and is hereby incorporated by reference in its entirety. Said XML copy, created on Jun. 16, 2025, is named 191016-011003_US_SL.xml and is 407,188 bytes in size.

### FIELD

Aspects of present disclosure relates to lipid nanoparticles (LNP) incorporating nucleic acids such as synthetic CpG oligodeoxynucleotides (ODNs) or single-stranded oligoribonucleotides (ORN) that activate Toll-like receptors (TLRs).

### BACKGROUND

The innate immune system has developed germline-encoded pattern recognition receptors (PRRs) that recognized conserved microbial pathogen-associated molecular patterns (PAMPs) and danger-associated molecular patterns (DAMPs). Ligand binding to PRRs triggers specific signaling pathways that initiate inflammatory responses tailored to the type of stimuli, whether it be sterile tissue damage or microbial in nature, such as bacterial, viral, or fungal. The innate immune system is a critical component of this surveillance system and central to the development of long-lived immunological memory.

Vaccines are critical in the prevention of infectious diseases and are foundational to protecting public health. Vaccines also show promise in therapeutic treatment of some types of cancer. Central to a vaccine's ability to induce protective humoral and cellular immunity is its activation of PRRs that initiates signaling cascades instructing the ensuing antigen-specific immune response and development of long-lived memory B and T cells. In the absence of sufficient PRR activation, immune responses are weak or tolerogenic, short-lived, and insufficient to provide robust protection against subsequent infection. Vaccines consisting of live attenuated or whole/partial inactivated bacteria and viruses inherently maintain their ability to engage with PRRs. As vaccine technology has improved and subunit or purified recombinant antigens have been developed, the specificity and safety profile of vaccines has improved. However, this has reduced or removed their ability to sufficiently activate the immune system, and thus additional adjuvants are required to improve immunogenicity. An adjuvant is any component that enhances the immune response by activating the innate immune system and increasing both the magnitude and quality of the antigen-specific adaptive immune response. The use of adjuvants allows for lower doses of antigen (dose sparing), increased immunogenicity in individuals with weakened immune systems (e.g. people that are immunocompromised or are >65 years of age), lowering the cost of goods and ease of distribution (Pulendran B et al. Nat Rev Drug Discov. 2021 June; 20(6):454-475, PMID: 33824489; Weinberger B. Curr Opin Pharmacol. 2018 August; 41:34-41. PMID: 29677646). Most approved adjuvants today have been in use for decades and are insufficient to provide robust protective immunity to many pathogens. Thus there is a demand for novel adjuvants that are more effective yet maintain a good safety profile.

A subset of PRRs have evolved to detect foreign nucleic acids. A class of PRRs, Toll-like receptors (TLRs), recognize various properties of microbial nucleic acids and are sequestered to the endolysosomal compartment as a means of differentiating between self and pathogen-derived nucleic acids (Kawai T, Akira S. Nat Immunol. 2006 February; 7(2):131-7. PMID: 16424890). Canonically, TLR3 recognizes double-stranded RNA, TLR7 and TLR8 recognize single-stranded RNA, and TLR9 recognizes unmethylated CpG dinucleotide motifs that are enriched in prokaryotic genomes compared to that of eukaryotes. There are also cytosolic PRRs such as retinoic acid-inducible gene I (RIG-I) and melanoma differentiation-associated protein 5 (MDA5) that canonically recognize uncapped 5′ triphosphate RNA (single-stranded and double-stranded) and long double-stranded RNA (dsRNA), respectively, that triggers anti-viral type I interferon signaling pathways. These PRR signaling pathways are of interest to vaccine adjuvant development because they elicit T_{H}1-type B and T cell responses that provide superior protection against intracellular viruses and bacteria (Yang J X et al. Pharmaceutics. 2022 Feb. 16; 14(2):423. PMID: 35214155; Pulendran B et al. Nat Rev Drug Discov. 2021 June; 20(6):454-475. PMID: 33824489). Natural and synthetic ligands to these TLRs have been developed that are potent adjuvants capable of driving antigen-specific memory B cells, long-lived plasma cells and memory T cells. Several adjuvants in this TLR class have been approved for clinical use. However, major hurdles still exist in translating many of these compounds to the clinic. Common challenges include unacceptable safety profiles due to poor therapeutic indexes, instability and unfavorable pharmacokinetics. There is extensive interest in developing delivery platforms to overcome these challenges and that are readily scalable to large scale production.

Immunostimulatory nucleic acids have been encapsulated in lipid nanoparticles (U.S. Pat. No. 8,354,522B2/RNA and U.S. Pat. No. 8,304,396B2/CpG (Coley); U.S. Ser. No. 10/568,958/CureVac; Wilson K D, Tam Y K. Expert Rev Clin Pharmacol. 2009 March; 2(2):181-93. PMID: 24410650) for a variety of applications, and in some instances for co-delivering antigens and adjuvants (Krishnamachari Y, Salem A K. Adv Drug Deliv Rev. 2009 Mar. 28; 61(3):205-17. PMID: 19272328). However, many of the formulations contained lipids like DOTAP for imparting a positive charge on the surface of the LNP to improve cellular uptake or relied on the natural tropism of the LNP to allow for relatively nonspecific and inefficient uptake by immune cells. Targeting of LNPs for more efficient presentation and activation of immune cells offers the promise of improving potency and decreasing toxicity of the LNPs.

Dinucleotide unmethylated CpG motifs common in prokaryotic DNA are natural ligands to TLR9. Synthetic CpG oligodeoxynucleotides (ODN) that are potent TLR9 agonists have been developed. In humans, TLR9 is only expressed by plasmacytoid DCs (pDCs) and B cells; in rodents TLR9 is more broadly expressed by monocytes, macrophages, and other DC populations (Desmet C J, Ishii K J. Nat Rev Immunol. 2012 Jun. 22; 12(7):479-91. PMID: 22728526.). Class A, B and C synthetic CpG ODNs have been described that differ in the number of CpG dinucleotides, flanking nucleotides, 5′ and 3′ motifs, and type of phosphate backbone, all of which influence the efficiency of uptake into the endolysosomal compartment and ligation with TLR9 (Bode C et al. Expert Rev Vaccines. 2011 April; 10(4):499-511. PMID: 21506647; Krieg A M. Annu Rev Immunol. 2002; 20:709-60. PMID: 11861616). CpG ODNs induce IL-12 and type I interferons that promote potent T_{H}1-type B cell isotype switching and CD8 T cell responses against intracellular bacterial and dsDNA viruses (Desmet C J, Ishii K J. Nat Rev Immunol. 2012 Jun. 22; 12(7):479-91. PMID: 22728526). These T_{H}1-type adaptive immune responses have been shown to be protective in many animal infection models. In humans, the class B CpG 1018 was recently approved for the hepatitis B virus vaccine Heplisav-B. Use of CpG 1018 with the HBV surface antigen HBsAg improved vaccine immunogenicity over Engerix-B, which uses the adjuvant aluminum hydroxide (alum) that skews toward a T_{H}2-type immune response with poor cellular immunity. Heplisav-B is able to achieve protective levels of antibodies after two immunizations compared to three for Engerix-B, and immunity is longer lasting (Campbell J D. Methods Mol Biol. 2017; 1494:15-27. PMID: 27718183). CpG 1018 also has a favorable vaccine adjuvant safety profile with low toxicity and off-target effects (European Medicines Agency, 10 Dec. 2020 CHMP Assessment Report, EMA/1767/202; Bode C et al. Expert Rev Vaccines. 2011 April; 10(4):499-511. PMID: 21506647).

Despite this success, the pharmacokinetics of CpG ODN are not ideal: due to the low molecular weight (CpG 1018 is 22 nucleotides long and 8.27 kDa), after intramuscular injection CpG rapidly diffuses out of the lymph and draining lymph nodes (dLN) into the circulation, where a significant proportion distributes in the kidney and liver (European Medicines Agency, 10 Dec. 2020 CHMP Assessment Report, EMA/1767/2021). This requires relatively large doses of CpG to be co-administered with the antigen. Importantly, the accumulation of CpG ODN in draining LNs after immunization influences the strength of the antigen-specific adaptive immune response. In a publication by Liu et al., following subcutaneous injection of CpG ODN in mice less than 0.3% was found to accumulate in the LNs (Liu H et al. Nature. 2014 Mar. 27; 507(7493):519-22. PMID: 24531764). By linking various lipophilic tails to CpG, CpG accumulation in the dLNs could be dramatically improved. When modified CpG was co-administered subcutaneously in mice with protein antigen, the magnitude of IFN-γ-producing antigen-specific CD8 T cells was as much as 10-fold greater than unmodified CpG. There was a strong correlation between the level of CpG in the dLNs and the expansion of antigen-specific CD8 T cells. Thus, improving the delivery and biodistribution of CpG into the dLNs increases vaccine immunogenicity and decreases the dose. The development of novel nanoparticle delivery platforms, such as lipid nanoparticles targeting innate immune cells, could further improve the bioactivity of CpG adjuvants while further improving their safety profile and extend their use to other vaccine indications.

Another class of nucleic acid adjuvants are TLR7 and 8 agonists. Similar to TLR9, TLR7 and TLR8 are located with the endosomal compartment and canonically recognize ssRNA. TLR7/8 activation results in type I interferon production as well as acute proinflammatory cytokines such as IL-6, TNF-α, IL-12, and IFN-γ depending on the cell type (Desmet C J, Ishii K J. Nat Rev Immunol. 2012 Jun. 22; 12(7):479-91. PMID: 22728526). Whereas TLR9 expression in humans is restricted to pDC and B cells, TLR7 and 8 are more broadly expressed by pDCs, B cells, conventional DCs, monocytes, macrophages and neutrophils. Natural viral RNA sequences have been identified as TLR7/8 agonists, and synthetic single-stranded oligoribonucleotides (ORNs) enriched with GU or AU motifs have been identified as potent agonists that preferentially activate TLR7 or TLR8 depending on the sequence (Forsbach A et al. J Immunol. 2008 Mar. 15; 180(6):3729-38. PMID: 18322178; Bourquin C et al. J Immunol. 2009 Nov. 15; 183(10):6078-86. PMID: 19890064). These ORNs can be synthesized with phosphorothioate linkages to improve resistance to nuclease degradation, but even in vitro they require delivery with liposomes (e.g. the cationic lipid DOTAP) to promote uptake into the endosomal compartment where TLR7 and 8 reside.

Multiple synthetic agonists such as imidazoquinoline derivatives (IMDs) agonists have been developed for the clinic (Bhagchandani S et al. Adv Drug Deliv Rev. 2021 August; 175:113803. PMID: 34058283). Several agonists have been approved for topical administration for the treatment of HPV-associated wart, actinic keratosis and basal cell carcinoma. However, given the broader expression of TLR7/8 compared to TLR9 and the potency of these agonists, translation of these compounds for other indications has been hampered with unacceptable toxicity and a narrow therapeutic index. Intramuscular and oral administration has been pursued for cancer immunotherapy and improvements in delivery have been made to decrease systemic toxicity. Use of synthetic agonists has shown promise as vaccine adjuvants using bioengineering approaches that complexes agonists with protein antigens, polymers or nanoparticles that improves local retention and reduces systemic exposure (Holbrook B C et al. Immunology. 2018 March; 153(3):357-367. PMID: 28940186; Bhagchandani S et al. Adv Drug Deliv Rev. 2021 August; 175:113803. PMID: 34058283).

There remains an unmet need for targeted lipid nanoparticles useful for improving the efficiency and specificity of ORN delivery to TLR7/8 innate immune cells in draining LNs with reduced off-target systemic effects.

### SUMMARY

Lipid nanoparticle (LNP) compositions are provided herein, and methods of making and using the same. In some embodiments, the present disclosure provides targeted lipid nanoparticle (LNP) compositions comprising nucleic acids. In some embodiments, the LNP compositions comprise nucleic acids that are recognized by pattern recognition receptors (PRRs) that recognized conserved microbial pathogen-associated molecular patterns (PAMPs) and danger-associated molecular patterns (DAMPs).

In some aspects, the PRRs can be certain Toll-like receptors (TLRs). For example, the nucleic acid can be: (1) a CpG oligodeoxynucleotide (ODN) recognized by Toll-like Receptor 9 (TLR9) (e.g., a TLR9 agonist), (2) a ssRNA oligonucleotide recognized by Toll-like Receptor 7 (TLR7) and/or Toll-like Receptor 8 (TLR8), (3) a nucleic acid recognized by retinoic acid-inducible gene-I-like receptors (RIG-I) like Receptor (RLR), (4) a dsRNA recognized by melanoma differentiation-associated protein 5 (MDA5), or other nucleic acid PRR sensors (including without limitation: NOD2, LGP2, DDX1-DDX21-DHX36, DDX60, DHX9, DHX36, DDX41, AIM2, IF16, ZBP1, LRRFIP1, STING).

In some embodiments, a dendritic-cell targeted LNP comprising one or more cationic ionizable lipid(s) is useful for delivery of CpG ODNs that are TLR9 agonists, for dendritic cell targeting or methods of using these LNP compositions as a vaccine for the prevention of tuberculosis or other mycobacterial infections. In some embodiments, a dendritic-cell targeted LNP comprising one or more cationic ionizable lipid(s) is useful for delivery of CpG ODNs that are TLR9 agonists, for dendritic cell targeting or methods of using these LNP compositions.

In some embodiments, the present disclosure provides targeted lipid nanoparticle (LNP) compositions to improve the efficiency and specificity of ORN delivery to TLR7/8 innate immune cells in draining LNs with reduced off-target systemic effects.

In some embodiments, the LNP composition comprises: (a) a TLR-activating nucleic acid; (b) an ionizable cationic lipid comprising a KC3 ionizable cationic lipid at a N/P ratio of 3 to 7 relative to the nucleic acid, the ionizable cationic lipid present in the LNP composition in a total amount of 46-54 mol % of a total lipid content of the LNP composition; (c) one or more phospholipids in a total amount of 5-20 mol % of the total lipid content of the LNP composition; (d) one or more anionic phospholipids in a total amount of 2-8 mol % of the total lipid content of the LNP composition; (e) a conjugated lipid in a total amount of 1-3.5 mol % of the total lipid content of the LNP composition; and (f) a sterol such cholesterol (e.g., in an amount providing the remainder of the LNP composition). In some embodiments, the TLR-activating nucleic acid is mRNA. In some embodiments, the TLR-activating nucleic acid is a DNA oligonucleotide.

In some embodiments, uses of LNP compositions comprising nucleic acids recognized by PRRs (e.g., TLRs) and related methods of treatment and manufacture include: (a) the use of the LNP as a vaccine adjuvant (e.g., to protect against pathogens such as viruses, bacteria or fungi, or to protect against drugs, natural or synthetic compounds such as opioids), (b) for cancer prevention, therapeutic tumor-associated antigens or neoadjuvant immunotherapy, or (c) as vaccines for targeting factors involved in non-communicable diseases such as cardiovascular disease, stroke or Alzheimer's Disease (AD), as well as the preparation or manufacture of medicaments for such uses or methods. Provided in some aspects of the disclosure is a pharmaceutical composition comprising the lipid nanoparticle described herein, and a pharmaceutically acceptable carrier. In some embodiments, the composition is a vaccine.

In some embodiments, a lipid nanoparticle (LNP) composition comprises a TLR-activating nucleic acid; an ionizable cationic lipid; a sterol; one or more phospholipids; and optionally further comprising a conjugated lipid. In some embodiments, a lipid nanoparticle (LNP) composition comprises a TLR-activating nucleic acid; an ionizable cationic lipid disclosed herein; cholesterol; one or more phospholipids selected from the group consisting of: DSPC, DPPC and DOPC; and a PS lipid selected from the group consisting of: DPPS, DSPS and DOPS; and optionally further comprising a conjugated lipid comprising PEG.

Aspects of the disclosure relate to a lipid nanoparticle (LNP) composition comprising a TLR-activating nucleic acid. Other aspects relate to a pharmaceutical composition comprising the lipid nanoparticle composition. In some embodiments the pharmaceutical composition is a vaccine.

In some embodiments, the TLR-activating nucleic acid is an RNA oligonucleotide or a DNA oligonucleotide. In some embodiments, the nucleic acid is selected from the group consisting of SEQ ID NO:118-SEQ ID NO:126 (Table 56) or SEQ ID NO:127-167 (Table 60). In some embodiments, the TLR-activating nucleic acid is a TLR9-activating DNA oligonucleotide and/or a TLR7/8-activating RNA oligonucleotide. In some embodiments, the LNP composition further comprises a TLR4 agonist. In some embodiments, the TLR4 agonist is selected from the group consisting of Monophosphoryl Lipid A (MPL), Monophosphoryl Lipid A-504, Monophosphoryl 3-Deacyl Lipid A, and Monophosphoryl Hexa-acyl Lipid A, and 3-Deacyl.

In some embodiments, the TLR-activating nucleic acid is a TLR9-activating oligonucleotide, for example CPG1018. In some embodiments, the TLR-activating nucleic acid comprises a TLR7 and/or TLR-8 activating RNA oligonucleotide.

In some embodiments, the TLR-activating nucleic acid comprises both a TLR-activating DNA oligonucleotide and a TLR-activating RNA oligonucleotide.

In some embodiments, the TLR-activating nucleic acid is a DNA comprising a CpG site. In some embodiments, the TLR-activating nucleic acid activates human TLR9. In some embodiments, the CpG is selected from the group consisting of: CpG-ODN 1018 (SEQ ID NO. 2), CpG-ODN 2006 (SEQ ID NO. 3; also known as ODN 7909 or PF3512676), CpG-ODN 2216 (SEQ ID NO. 6), CpG-ODN 2336 (SEQ ID NO. 7) and CpG-ODN 2395 (SEQ ID NO. 8).

In some embodiments, the TLR-activating nucleic acid comprises a TLR7 and/or TLR-8 activating nucleic acid selected from the group consisting of: R-0006 (SEQ ID NO. 25), R-1075 (SEQ ID NO. 11), 9.2dr (SEQ ID NO. 212), 9.3as (SEQ ID NO. 113), 9.2s (SEQ ID NO. 114) and HIV sequence 1 (SEQ ID NO. 116).

In some embodiments, the TLR-activating nucleic acid activates RIG-I. In some embodiments, the TLR-activating nucleic acid is selected from the group consisting of: 3P-GFP2 (SEQ ID NO. 127) annealed with either SEQ ID NO. 128 or SEQ ID NO. 129, 3P-A24 (SEQ ID NO. 130) annealed with either SEQ ID NO. 133 or SEQ ID NO. 134, 3P-G24 (SEQ ID NO. 130) annealed with either SEQ ID NO. 140 or SEQ ID NO. 141, 3P-C24 (SEQ ID NO. 130) annealed with either SEQ ID NO. 147 or SEQ ID NO. 148 and 3P-U24 (SEQ ID NO. 151) annealed with either SEQ ID NO. 154 or SEQ ID NO. 155.

Aspects of the disclosure relate to a (LNP) composition comprising or consisting of: (a) a nucleic acid antigen recognized by a germline-encoded pattern recognition receptor (PRR); (b) an ionizable cationic lipid comprising an ionizable cationic lipid at a N/P ratio of 4 to 6 relative to the mRNA, the ionizable cationic lipid present in the LNP composition in a total amount of 46-54 mol % of a total lipid content of the LNP composition; (c) one or more phospholipids selected from the group consisting of distearoylphosphatidylcholine (DSPC), hydrogenated soy phosphatidylcholine (HSPC), and dipalmitoylphosphatidylcholine (DPPC), in a total amount of 10-18 mol % of the total lipid content of the LNP composition; (d) one or more anionic phospholipids selected from the group consisting of dipalmitoylphosphatidyl-L-serine (DPPS), or distearoylphosphatidyl-L-serine (DSPS), distearoylphosphatidylglycerol (DSPG), and dipalmitoyphosphatidylglycerol (DPPG) in a total amount of 2-8 mol % of the total lipid content of the LNP composition; (e) PEG(2000)-dimyristoylglycerol (PEG-DMG) in a total amount of 1-3.5 mol % of the total lipid content of the LNP composition; and (f) cholesterol.

Other aspects of the disclosure relate to a pharmaceutical composition comprising the lipid nanoparticle composition. In some embodiments the pharmaceutical composition is a vaccine.

In some embodiments, the PRR recognizes a conserved microbial pathogen-associated molecular patterns (PAMPs) and danger-associated molecular patterns (DAMPs).

In some embodiments, the nucleic acid antigen is a CpG oligodeoxynucleotide recognized by TLR9.

In some embodiments, the nucleic acid antigen is a ssRNA oligonucleotide recognized by TLR7 or TLR8.

In some embodiments, the nucleic acid antigen is a ssRNA oligonucleotide recognized by TLR7 and TLR8.

In some embodiments, the nucleic acid antigen is a RIG-I recognizes uncapped 5′ triphosphate RNA (aka pppRNA, 3pRNA). In some embodiments, the nucleic acid antigen is a 5′ppp ssRNA with poly-U/UG, poly-U/UC, poly-A/AG. In some embodiments, the nucleic acid antigen comprises RNA regions enriched with polyuridine and interspersed guanosines (poly-U/UG), polyuridine with interspersed cytidines, polyadenosine interspersed with guanosines.

In some embodiments, the nucleic acid antigen encodes MDA5 (melanoma differentiation-associated protein 5).

In some embodiments, the nucleic acid antigen is selected from the group consisting of NOD2, LGP2, DDX1-DDX21-DHX36, DDX60, DHX9, DHX36, DDX41, AIM2, IF16, ZBP1, LRRFIP1, and STING.

In some embodiments, the nucleic acid antigen is a CpG oligodeoxynucleotide (ODN) 1018 (SEQ ID NO: 2).

In some embodiments, the nucleic acid antigen is a CpG-ODN 2006 (SEQ ID NO: 3). In some embodiments, the nucleic acid antigen is TLR7/8 ssRNA oligonucleotide 5′-UUGUUGUUGUUGUUGUUGUU-3′ (SEQ ID NO: 25).

In some embodiments, the nucleic acid antigen is mRNA encoding for SARS-CoV-2 Spike protein (SEQ ID NO: 1).

In some embodiments, the nucleic acid antigen is selected from the group consisting of SEQ ID NO:1, SEQ ID NO:2, or SEQ ID NO:3.

In some embodiments, the nucleic acid antigen is a TLR-9 CpG-ODN adjuvant.

In some embodiments, the nucleic acid antigen is selected from the group consisting of SEQ ID NO:11-SEQ ID NO:117 (Table 55).

### BRIEF DESCRIPTION OF THE DRAWINGS

FIG. 1 Oxidative degradation of liposomes containing KC3 (DLin-KC3-DMA), a polyunsaturated ICL with a single methylene between two olefins, to liposomes containing ICLs with monounsaturated alkyl chains (KC3-OA, KC3-PA, or KC3-C17(C8:1)) and the fully saturated ICL, KC3-C17. Effect of hydrogen peroxide on the stability of individual ionizable cationic lipids measured by CAD-HPLC.

FIG. 2 Comparison of the mCherry expression in murine dendritic cells of LNPs containing the polyunsaturated KC3, the monounsaturated KC3-OA, KC3-PA, or KC3C17(C8:1), and the fully saturated KC3C17, all with or without DPPS (NH₄⁺ salt), at 0.3 or 1 μg/mL mRNA for 24 h. UT sample corresponds to cells where no LNPs were added.

FIG. 3 Comparison of the mCherry expression in human dendritic cells of LNPs containing the polyunsaturated KC2 or KC3 with a single methylene between two olefins, polyunsaturated KC3-01 with four methylenes between two olefins, monounsaturated KC3-OA, KC3-PA, or KC3C17(C8:1), and ALC-0315, all with except ALC-0315 with DPPS (NH₄⁺ salt), at 0.1 or 1 μg/mL mRNA for 24 h. Untreated samples correspond to cells where no LNPs were added.

FIG. 4 Comparison of the mCherry expression of LNP formulations with 5 mol % DSPS and 46-54 mol % of KC3-OA to ALC-0315 and SM-102 LNP controls, at 0.1 and 1 μg/mL mRNA for 24 h in human dendritic cells. Untreated DC sample corresponds to human dendritic cells where no LNPs were added.

FIG. 5 Comparison of the mCherry expression of LNP formulations with 0 or 5 mol % DSPS and 50 mol % KC2-01 at N/P ratios of 4-7, at 0.1 μg/mL mRNA for 24 h in human dendritic cells. These were also compared to LNPs containing KC3-OA and 5 mol % DSPS at N/P of 5. Untreated DC sample corresponds to human dendritic cells where no LNPs were added.

FIG. 6A Comparison of the mCherry expression of UO-1 or KC3-01 containing LNP formulations with 0-10 mol % of DSPG in human dendritic cells following incubation for 24 h at 1 μg/mL mRNA. ALC-0315 and SM-102 LNPs controls were also included at 1 μg/mL mRNA and untreated DC sample corresponds to human dendritic cells where no LNPs were added.

FIG. 6B Comparison of the mCherry expression of UO-1 or KC3-01 containing LNP formulations with 0-10 mol % of DSPG in human dendritic cells following incubation for 24 h at 0.1 μg/mL mRNA. ALC-0315 and SM-102 LNPs controls were also included at 0.1 μg/mL mRNA and untreated DC sample corresponds to human dendritic cells where no LNPs were added.

FIG. 7A Comparison of the mCherry expression of 48 mol % KC3-OA containing LNP formulations with 5 mol % of various anionic phospholipids in human dendritic cells following incubation for 24 h at 1 μg/mL mRNA. All LNPs included 2.5 mol % of DSPC, 50 mol % of UO-1, and 1.5 mol % of PEG-DMG. The anionic phospholipids included the phosphatidylglycerols, DOPG, DSPG, DPPG, and DMPG, as well as DSPS. In some LNPs, the DSPG and DSPS were combined either alone or together with DSPC. Two donors were used to produce human dendritic cells in this study and untreated DC sample corresponds to human dendritic cells where no LNPs were added.

FIG. 7B Comparison of the mCherry expression of 48 mol % KC3-OA containing LNP formulations with 5 mol % of various anionic phospholipids in human dendritic cells following incubation for 24 h at 0.1 μg/mL mRNA. All LNPs included 2.5 mol % of DSPC, 50 mol % of UO-1, and 1.5 mol % of PEG-DMG. The anionic phospholipids included the phosphatidylglycerols, DOPG, DSPG, DPPG, and DMPG, as well as DSPS. In some LNPs, the DSPG and DSPS were combined either alone or together with DSPC. Two donors were used to produce human dendritic cells in this study and untreated DC sample corresponds to human dendritic cells where no LNPs were added.

FIG. 8 Comparison of the mCherry expression in murine dendritic cells of LNPs containing KC3-OA LNPs with either 5 mol % DSPS (Na⁺ salt) or 5 mol % DPPS (NH₄⁺ salt) after incubation at 1 μg/mL mRNA for 24 h. ALC-0315 and SM-102 LNPs controls were also included at 1 μg/mL mRNA. UT sample corresponds to cells where no LNPs were added.

FIG. 9A is a scheme showing the synthesis of 2-((S)-2,2-di((6Z,12Z)-octadeca-6,12-dien-1-yl)-1,3-dioxolan-4-yl)-N,N-dimethylethan-1-amine (AKG-KC2-01) and 3-((S)-2,2-di((6Z,12Z)-octadeca-6,12-dien-1-yl)-1,3-dioxolan-4-yl)-N,N-dimethylpropan-1-amine (AKG-KC3-01) according to some embodiments of the disclosure.

FIG. 9B is a scheme showing the synthesis 2-((S)-2,2-di((Z)-octadec-9-en-1-yl)-1,3-dioxolan-4-yl)-N,N-dimethylethan-1-amine (AKG-KC2-OA), 2-((S)-2,2-di((Z)-hexadec-9-en-1-yl)-1,3-dioxolan-4-yl)-N,N-dimethylethan-1-amine (AKG-KC2-PA), 3-((S)-2,2-di((Z)-octadec-9-en-1-yl)-1,3-dioxolan-4-yl)-N,N-dimethylpropan-1-amine (AKG-KC3-OA), and 3-((S)-2,2-di((Z)-hexadec-9-en-1-yl)-1,3-dioxolan-4-yl)-N,N-dimethylpropan-1-amine, (AKG-KC3-PA, 0-12418) according to some embodiments of the disclosure.

FIG. 9C is a scheme showing the synthesis of 3-((S)-2,2-di((Z)-heptadec-8-en-1-yl)-1,3-dioxolan-4-yl)-N,N-dimethylpropan-1-amine, AKG-KC3-C17(C8:1) and (S)-3-(2,2-diheptadecyl-1,3-dioxolan-4-yl)-N,N-dimethylpropan-1-amine, AKG-KC3-C17 according to some embodiments of the disclosure.

FIG. 9D is a reaction scheme for the preparation of a KC3-X1 ionizable lipid.

FIG. 9E is a reaction scheme for the preparation of a KC3-X2 ionizable lipid.

FIG. 10 is a schematic for an in vivo murine inflammatory profile of a synthetic nucleic acid TLR9 agonist delivered by a PS targeted lipid nanoparticle (Example 22).

FIG. 11A is a graph showing INF-α detected in plasma of mice in an in vivo murine inflammatory profile of a synthetic nucleic acid TLR9 agonist delivered by a PS targeted lipid nanoparticle (Example 22).

FIG. 11B is a graph showing INF-α detected in draining lymph nodes of mice in an in vivo murine inflammatory profile of a synthetic nucleic acid TLR9 agonist delivered by a PS targeted lipid nanoparticle (Example 22).

FIG. 12A is a graph showing IFN-γ detected in plasma of mice in an in vivo murine inflammatory profile of a synthetic nucleic acid TLR9 agonist delivered by a PS targeted lipid nanoparticle (Example 22).

FIG. 12B is a graph showing IFN-γ detected in draining lymph nodes of mice in an in vivo murine inflammatory profile of a synthetic nucleic acid TLR9 agonist delivered by a PS targeted lipid nanoparticle (Example 22).

FIG. 13A is a graph showing IL-6 detected in plasma of mice in an in vivo murine inflammatory profile of a synthetic nucleic acid TLR9 agonist delivered by a PS targeted lipid nanoparticle (Example 22).

FIG. 13B is a graph showing IL-6 detected in draining lymph nodes of mice in an in vivo murine inflammatory profile of a synthetic nucleic acid TLR9 agonist delivered by a PS targeted lipid nanoparticle (Example 22).

FIG. 14A is a graph showing IL-12p70 detected in plasma of mice in an in vivo murine inflammatory profile of a synthetic nucleic acid TLR9 agonist delivered by a PS targeted lipid nanoparticle (Example 22).

FIG. 14B is a graph showing IL-12p70 detected in draining lymph nodes of mice in an in vivo murine inflammatory profile of a synthetic nucleic acid TLR9 agonist delivered by a PS targeted lipid nanoparticle (Example 22).

FIG. 15A is a graph showing chemokine CCL2/MCP-1 detected in plasma of mice in an in vivo murine inflammatory profile of a synthetic nucleic acid TLR9 agonist delivered by a PS targeted lipid nanoparticle (Example 22).

FIG. 15B is a graph showing chemokine CCL2/MCP-1 detected in draining lymph nodes of mice in an in vivo murine inflammatory profile of a synthetic nucleic acid TLR9 agonist delivered by a PS targeted lipid nanoparticle (Example 22).

FIG. 16 is a graph showing TNF-α detected in plasma and draining lymph nodes of mice in an in vivo murine inflammatory profile of a synthetic nucleic acid TLR9 agonist delivered by a PS targeted lipid nanoparticle (Example 22).

FIG. 17A is a graph showing levels of cytokine IL-6 in the blood of mice observed in the in vivo inflammatory profile of a synthetic nucleic acid TLR 7/8 agonist delivered by a PS-targeted nanoparticle (Example 21).

FIG. 17B is a graph showing levels of cytokine TL-6 in the draining lymph nodes of mice observed in the in vivo inflammatory profile of a synthetic nucleic acid TLR 7/8 agonist delivered by a PS-targeted nanoparticle (Example 21).

FIG. 18A is a graph showing levels of IL-1β in the blood of mice observed in the in vivo inflammatory profile of a synthetic nucleic acid TLR 7/8 agonist delivered by a PS-targeted nanoparticle (Example 21).

FIG. 18B is a graph showing levels of IL-1β in the draining lymph nodes of mice observed in the in vivo inflammatory profile of a synthetic nucleic acid TLR 7/8 agonist delivered by a PS-targeted nanoparticle (Example 21).

FIG. 19A is a graph showing levels of TNF-α in the blood of mice observed in the in vivo inflammatory profile of a synthetic nucleic acid TLR 7/8 agonist delivered by a PS-targeted nanoparticle (Example 21).

FIG. 19B is a graph showing levels of TNF-α in the draining lymph nodes of mice observed in the in vivo inflammatory profile of a synthetic nucleic acid TLR 7/8 agonist delivered by a PS-targeted nanoparticle (Example 21).

FIG. 20A is a graph showing levels of IFN-γ in the blood of mice observed in the in vivo inflammatory profile of a synthetic nucleic acid TLR 7/8 agonist delivered by a PS-targeted nanoparticle (Example 21).

FIG. 20B is a graph showing levels of IFN-γ in the draining lymph nodes of mice observed in the in vivo inflammatory profile of a synthetic nucleic acid TLR 7/8 agonist delivered by a PS-targeted nanoparticle (Example 21).

FIG. 21A is a graph showing levels of keratinocyte chemoattractant (KC) in the blood of mice observed in the in vivo inflammatory profile of a synthetic nucleic acid TLR 7/8 agonist delivered by a PS-targeted nanoparticle (Example 21).

FIG. 21B is a graph showing levels of keratinocyte chemoattractant (KC) in the draining lymph nodes of mice observed in the in vivo inflammatory profile of a synthetic nucleic acid TLR 7/8 agonist delivered by a PS-targeted nanoparticle (Example 21).

FIG. 22A is a graph showing levels of monocyte chemoattractant CCL2 in the blood of mice observed in the in vivo inflammatory profile of a synthetic nucleic acid TLR 7/8 agonist delivered by a PS-targeted nanoparticle (Example 21).

FIG. 22B is a graph showing levels of monocyte chemoattractant CCL2 in the draining lymph nodes of mice observed in the in vivo inflammatory profile of a synthetic nucleic acid TLR 7/8 agonist delivered by a PS-targeted nanoparticle (Example 21).

FIG. 23A is a graph showing levels of CXCL10 in the blood of mice observed in the in vivo inflammatory profile of a synthetic nucleic acid TLR 7/8 agonist delivered by a PS-targeted nanoparticle (Example 21).

FIG. 23B is a graph showing levels of CXCL10 in the draining lymph nodes of mice observed in the in vivo inflammatory profile of a synthetic nucleic acid TLR 7/8 agonist delivered by a PS-targeted nanoparticle (Example 21).

FIG. 24A is a graph showing the concentration of IFN-γ in the serum of mice in an in vivo profile inflammatory profile of a synthetic nucleic acid TLR9 agonist delivered by PS-targeted lipid nanoparticles (Example 27).

FIG. 24B is a graph showing the concentration of IFN-γ in the draining lymph nodes (dLNs) of mice in an in vivo profile inflammatory profile of a synthetic nucleic acid TLR9 agonist delivered by PS-targeted lipid nanoparticles (Example 27).

FIG. 25A is a graph showing the concentration of TNF-α in the serum of mice in an in vivo profile inflammatory profile of a synthetic nucleic acid TLR9 agonist delivered by PS-targeted lipid nanoparticles (Example 27).

FIG. 25B is a graph showing the concentration of TNF-α in the draining lymph nodes (dLNs) of mice in an in vivo profile inflammatory profile of a synthetic nucleic acid TLR9 agonist delivered by PS-targeted lipid nanoparticles (Example 27).

FIG. 26A is a graph showing the concentration of IL-12p70 in the serum of mice in an in vivo profile inflammatory profile of a synthetic nucleic acid TLR9 agonist delivered by PS-targeted lipid nanoparticles (Example 27).

FIG. 26B is a graph showing the concentration of IL-12p70 in the draining lymph nodes (dLNs) of mice in an in vivo profile inflammatory profile of a synthetic nucleic acid TLR9 agonist delivered by PS-targeted lipid nanoparticles (Example 27).

FIG. 27A is a graph showing the concentration of IFN-α in the serum of mice in an in vivo profile inflammatory profile of a synthetic nucleic acid TLR9 agonist delivered by PS-targeted lipid nanoparticles (Example 27).

FIG. 27B is a graph showing the concentration of IFN-α in the draining lymph nodes (dLNs) of mice in an in vivo profile inflammatory profile of a synthetic nucleic acid TLR9 agonist delivered by PS-targeted lipid nanoparticles (Example 27).

FIG. 28A is a graph showing the concentration of IL-6 in the serum of mice in an in vivo profile inflammatory profile of a synthetic nucleic acid TLR9 agonist delivered by PS-targeted lipid nanoparticles (Example 27).

FIG. 28B is a graph showing the concentration of IL-6 in the draining lymph nodes (dLNs) of mice in an in vivo profile inflammatory profile of a synthetic nucleic acid TLR9 agonist delivered by PS-targeted lipid nanoparticles (Example 27).

FIG. 29A is a graph showing the concentration of IL-1β in the serum of mice in an in vivo profile inflammatory profile of a synthetic nucleic acid TLR9 agonist delivered by PS-targeted lipid nanoparticles (Example 27).

FIG. 29B is a graph showing the concentration of IL-1β in the draining lymph nodes (dLNs) of mice in an in vivo profile inflammatory profile of a synthetic nucleic acid TLR9 agonist delivered by PS-targeted lipid nanoparticles (Example 27).

FIG. 30A is a graph showing the concentration of IL-10 in the serum of mice in an in vivo profile inflammatory profile of a synthetic nucleic acid TLR9 agonist delivered by PS-targeted lipid nanoparticles (Example 27).

FIG. 30B is a graph showing the concentration of IL-10 in the draining lymph nodes (dLNs) of mice in an in vivo profile inflammatory profile of a synthetic nucleic acid TLR9 agonist delivered by PS-targeted lipid nanoparticles (Example 27).

FIG. 31A is a graph showing the concentration of GM-CSF in the serum of mice in an in vivo profile inflammatory profile of a synthetic nucleic acid TLR9 agonist delivered by PS-targeted lipid nanoparticles (Example 27).

FIG. 31B is a graph showing the concentration of GM-CSF in the draining lymph nodes (dLNs) of mice in an in vivo profile inflammatory profile of a synthetic nucleic acid TLR9 agonist delivered by PS-targeted lipid nanoparticles (Example 27).

FIG. 32A is a graph showing the concentration of KC in the serum of mice in an in vivo profile inflammatory profile of a synthetic nucleic acid TLR9 agonist delivered by PS-targeted lipid nanoparticles (Example 27).

FIG. 32B is a graph showing the concentration of KC in the draining lymph nodes (dLNs) of mice in an in vivo profile inflammatory profile of a synthetic nucleic acid TLR9 agonist delivered by PS-targeted lipid nanoparticles (Example 27).

FIG. 33A is a graph showing the concentration of MCP-1 (CCL2) in the serum of mice in an in vivo profile inflammatory profile of a synthetic nucleic acid TLR9 agonist delivered by PS-targeted lipid nanoparticles (Example 27).

FIG. 33B is a graph showing the concentration of MCP-1 (CCL2) in the draining lymph nodes (dLNs) of mice in an in vivo profile inflammatory profile of a synthetic nucleic acid TLR9 agonist delivered by PS-targeted lipid nanoparticles (Example 27).

FIG. 34A is a graph showing the concentration of IP-10 (CXCL10) in the serum of mice in an in vivo profile inflammatory profile of a synthetic nucleic acid TLR9 agonist delivered by PS-targeted lipid nanoparticles (Example 27).

FIG. 34B is a graph showing the concentration of IP-10 (CXCL10) in the draining lymph nodes (dLNs) of mice in an in vivo profile inflammatory profile of a synthetic nucleic acid TLR9 agonist delivered by PS-targeted lipid nanoparticles (Example 27).

FIG. 35A is a graph showing the concentration of RANTES (CCL5) in the serum of mice in an in vivo profile inflammatory profile of a synthetic nucleic acid TLR9 agonist delivered by PS-targeted lipid nanoparticles (Example 27).

FIG. 35B is a graph showing the concentration of RANTES (CCL5) in the draining lymph nodes (dLNs) of mice in an in vivo profile inflammatory profile of a synthetic nucleic acid TLR9 agonist delivered by PS-targeted lipid nanoparticles (Example 27).

FIG. 36A is a graph showing mCherry expression in MutuDC1940 cells 24 hrs after transfection with 0.3 ug/mL mCherry mRNA encapsulated in LNPs using the KC-OA series of ICLs.

FIG. 36B is a graph showing mCherry expression in MutuDC1940 cells 24 hrs after transfection with 0.1 ug/mL mCherry mRNA encapsulated in LNPs using the KC-OA series of ICLs.

FIG. 37A is a graph showing the concentration of IL-6 in the serum of mice in an in vivo profile inflammatory profile of a UUGU oligoribonucleotide to TLR7/8 located intracellularly in endolysosomal compartments (Example 28).

FIG. 37B is a graph showing the concentration of IL-6 in the draining lymph nodes (dLNs) of mice in an in vivo profile inflammatory profile of a s UUGU oligoribonucleotide to TLR7/8 located intracellularly in endolysosomal compartments (Example 28).

FIG. 38A is a graph showing the concentration of IL-1β in the serum of mice in an in vivo profile inflammatory profile of a UUGU oligoribonucleotide to TLR7/8 located intracellularly in endolysosomal compartments (Example 28).

FIG. 38B is a graph showing the concentration of IL-1β in the draining lymph nodes (dLNs) of mice in an in vivo profile inflammatory profile of a s UUGU oligoribonucleotide to TLR7/8 located intracellularly in endolysosomal compartments (Example 28).

FIG. 39A is a graph showing the concentration of TNF-α in the serum of mice in an in vivo profile inflammatory profile of a UUGU oligoribonucleotide to TLR7/8 located intracellularly in endolysosomal compartments (Example 28).

FIG. 39B is a graph showing the concentration of TNF-α in the draining lymph nodes (dLNs) of mice in an in vivo profile inflammatory profile of a s UUGU oligoribonucleotide to TLR7/8 located intracellularly in endolysosomal compartments (Example 28).

FIG. 40A is a graph showing the concentration of IFN-γ in the serum of mice in an in vivo profile inflammatory profile of a UUGU oligoribonucleotide to TLR7/8 located intracellularly in endolysosomal compartments (Example 28).

FIG. 40B is a graph showing the concentration of IFN-γ in the draining lymph nodes (dLNs) of mice in an in vivo profile inflammatory profile of a s UUGU oligoribonucleotide to TLR7/8 located intracellularly in endolysosomal compartments (Example 28).

FIG. 41A is a graph showing the concentration of KC in the serum of mice in an in vivo profile inflammatory profile of a UUGU oligoribonucleotide to TLR7/8 located intracellularly in endolysosomal compartments (Example 28).

FIG. 41B is a graph showing the concentration of KC in the draining lymph nodes (dLNs) of mice in an in vivo profile inflammatory profile of a s UUGU oligoribonucleotide to TLR7/8 located intracellularly in endolysosomal compartments (Example 28).

FIG. 42A is a graph showing the concentration of MCP-1/CCL2 in the serum of mice in an in vivo profile inflammatory profile of a UUGU oligoribonucleotide to TLR7/8 located intracellularly in endolysosomal compartments (Example 28).

FIG. 42B is a graph showing the concentration of MCP-1/CCL2 in the draining lymph nodes (dLNs) of mice in an in vivo profile inflammatory profile of a s UUGU oligoribonucleotide to TLR7/8 located intracellularly in endolysosomal compartments (Example 28).

FIG. 43A is a graph showing the concentration of IP-10/CXCL10 in the serum of mice in an in vivo profile inflammatory profile of a UUGU oligoribonucleotide to TLR7/8 located intracellularly in endolysosomal compartments (Example 28).

FIG. 43B is a graph showing the concentration of IP-10/CXCL10 in the draining lymph nodes (dLNs) of mice in an in vivo profile inflammatory profile of a s UUGU oligoribonucleotide to TLR7/8 located intracellularly in endolysosomal compartments (Example 28).

FIG. 44A is a graph showing the concentration of IFN-α in the serum of mice in an in vivo profile inflammatory profile of a UUGU oligoribonucleotide to TLR7/8 located intracellularly in endolysosomal compartments (Example 28).

FIG. 44B is a graph showing the concentration of IFN-α in the draining lymph nodes (dLNs) of mice in an in vivo profile inflammatory profile of a s UUGU oligoribonucleotide to TLR7/8 located intracellularly in endolysosomal compartments (Example 28).

FIG. 45 is a graph showing the concentration of IFN-β in the draining lymph nodes (dLNs) of mice in an in vivo profile inflammatory profile of a s UUGU oligoribonucleotide to TLR7/8 located intracellularly in endolysosomal compartments (Example 28).

FIG. 46 is a scheme showing the conjugation of TLR7/8 Agonist 1 (1a) and TLR7/8 Agonist 6 (1b) to PEG-DSPE.

FIGS. 47A-47C Quantification of VZV gE-specific CD4 T cells after vaccination with CpG-1018 or UUGU oligonucleotide adjuvants delivered by LNPs. Mice were prime/boost vaccinated with gE antigen and various adjuvants. Splenic T cell responses were quantified 12 days after the boost. FIG. 47A shows the percentage of gE-specific CD4 T cells in mice vaccinated with CpG or AS01 adjuvants. FIG. 47B shows the percentage of gE-specific CD4 T cells in mice vaccinated with CpG or UUGU adjuvants. FIG. 47C shows the proportion of gE-specific CD4 T cells that produce both IFN-g and IL-2, IFN-g alone, or IL-2 alone. SP, single positive.

FIGS. 48A-48B Quantification of VZV gE-specific IgG antibodies after vaccination with CpG-1018 or UUGU oligonucleotide adjuvants delivered by LNPs. Mice were prime/boost vaccinated with gE antigen and various adjuvants. Serum antibody titers were measured 12 days after the boost. FIG. 48A compares total gE-binding IgG titers induced by various CpG-1018 and AS01 adjuvants. Left panel, comparison between CpG-treated groups. Right panel, comparison between CpG- and AS01-treated groups. FIG. 48B compares total gE-binding IgG titers induced by various UUGU and AS01 adjuvants. Left panel, comparison between UUGU-treated groups; One-way ANOVA statistical comparisons were made between free UUGU and all other groups. Right panel, comparison between UUGU- and AS01-treated groups; One-way ANOVA statistical comparisons were made between AS01 (2 ug) and all other groups.

FIGS. 49A-49H Concentrations of pro-inflammatory cytokines and chemokines in the blood 6 hrs after VZV gE immunization of mice with various CpG adjuvants (left panels) or UUGU adjuvants (right panels). FIG. 49A graphs show IFN-gamma plasma concentrations. FIG. 49B graphs show TNF-alpha plasma concentrations. FIG. 49C graphs show IL-12p70 plasma concentrations. FIG. 49D graphs show IL-1beta plasma concentrations. FIG. 49E graphs show IFN-alpha plasma concentrations. FIG. 49F graphs show IFN-beta plasma concentrations. FIG. 49G graphs show CXCL1 plasma concentrations. FIG. 49H graphs show CXCL10 plasma concentrations.

FIG. 50 Quantification of hepatitis B surface antigen (HBsAg)-specific IgG antibodies after vaccination with CpG-1018 or UUGU oligonucleotide adjuvants delivered by PS-targeted LNPs. Mice were vaccinated with HBsAg and various CpG or UUGU adjuvants. On day 24 post-vaccination, serum antibody titers were measured.

FIG. 51 Quantification of respiratory syncytial virus prefusion-stabilized F (RSVpreF)-specific IgG antibodies after vaccination with CpG-1018 oligonucleotide adjuvants delivered by PS-targeted LNPs. Mice were vaccinated with RSVpreF antigen and various CpG adjuvants. On day 25 post-vaccination, serum antibody titers were measured.

### DETAILED DESCRIPTION

In some embodiments, the compounds and compositions described herein promote or provide delivery of TLR agonist nucleic acids to boost the antigen specific immune responses against infectious viruses or bacteria when co-administered with an antigen to elicit the desired immune response to protect against corresponding infections is a result. In some embodiments, the nucleic acid is a synthetic nucleic acid. In some embodiments the composition is an adjuvant comprises one or more nucleic acid oligonucleotides encoding one or more TLR-activating protein(s). In some embodiments the adjuvant uses a combination of TLR-activating nucleic acid oligonucleotides. In some embodiments the TLR-activating nucleic acids activate TLR7, TLR8, TLR9, or RIG-I. In some embodiments the TLR-activating nucleic acids have phosphorothioate linkages between nucleic acids. In some aspects, the nucleic acid sequence encodes a polypeptide that is recognized by T cells. Peptide fragments can be generated from an antigen that are recognized by T cell receptors. In some embodiments the nucleotides are linked together with phosphorothioate linkages, and/or nucleic acid sequences encoding polypeptides capable of activating TLR7, TLR8, TLR9, TLR3, or RIG-I.

In some embodiments, the compounds and compositions described herein promote efficient uptake and transfection of target cells, including tissue macrophages and dendritic cells.

In some embodiments, a lipid nanoparticle (LNP) composition comprises an ionizable cationic lipid, sterol, one or more phospholipids comprising at least one anionic phospholipid, a conjugated lipid and one or more nucleic acid sequences capable of activating TLR7, TLR8, TLR9, TLR3, or RIG-I. In some embodiments, a lipid nanoparticle (LNP) composition comprises an ionizable cationic lipid, sterol, one or more phospholipids comprising at least one anionic phospholipid, a conjugated lipid and one or more nucleic acid sequences agonizing TLR9 or TLR7/8. In some embodiments, a lipidic nanoparticle composition comprises lipids and nucleic acids, the lipidic nanoparticles comprising a compounds disclosed herein, combinations thereof or pharmaceutically acceptable salts thereof.

It is to be understood that both the foregoing general description and the following detailed description are exemplary and explanatory only and are not restrictive of the compositions and methods of the present disclosure.

### (I) DEFINITIONS

For convenience, certain terms employed in the specification, examples, and appended claims are collected here. Unless defined otherwise, all technical and scientific terms used herein have the same meaning as commonly understood by one of ordinary skill in the art to which this disclosure belongs.

As used herein, the following terms and phrases are intended to have the following meanings:

The articles “a” and “an” are used herein to refer to one or to more than one (i.e., to at least one) of the grammatical object of the article. By way of example, “an element” means one element or more than one element.

As used herein the term “comprising” or “comprises” is used in reference to compositions, methods, and respective component(s) thereof, that are present in a given embodiment, yet open to the inclusion of unspecified elements.

As used herein the term “consisting essentially of” refers to those elements required for a given embodiment. The term permits the presence of additional elements that do not materially affect the basic and novel or functional characteristic(s) of that embodiment of the disclosure.

The term “consisting of” refers to compositions, methods, and respective components thereof as described herein, which are exclusive of any element not recited in that description of the embodiment.

The term “comprising” when used in the specification includes “consisting of” and “consisting essentially of”.

If it is referred to “as mentioned above” or “mentioned above”, “supra” within the description it is referred to any of the disclosures made within the specification in any of the preceding pages.

If it is referred to “as mentioned herein”, “described herein”, “provided herein,” or “as mentioned in the present text,” or “stated herein” within the description it is referred to any of the disclosures made within the specification in any of the preceding or subsequent pages.

As used herein, the term “about” means acceptable variations within 20%, within 10% and within 5% of the stated value. In certain embodiments, “about” can mean a variation of +/−1%, 2%, 3%, 4%, 5%, 10% or 20%.

An “antigen” according to aspects of the disclosure covers any substance that will elicit an immune response. In particular, an “antigen” relates to any substance, preferably a peptide or protein, that reacts specifically with antibodies or T-lymphocytes (T cells). As used herein, the term “antigen” comprises any molecule which comprises at least one epitope. Preferably, an antigen in the context of the present disclosure is a molecule which, optionally after processing, induces an immune reaction, which is preferably specific for the antigen (including cells expressing the antigen). According to aspects of the present disclosure, any suitable antigen may be used, which is a candidate for an immune reaction, wherein the immune reaction is preferably a cellular immune reaction. In the context of the embodiments of the present disclosure, the antigen is presented by a cell, for example by an antigen presenting cell which includes a diseased cell, in particular a cancer cell, in the context of MHC molecules, which results in an immune reaction against the antigen. An antigen can be a product which corresponds to or is derived from a naturally occurring antigen. Such naturally occurring antigens may include tumor antigens.

As used herein, an “antigen peptide” refers to a portion or fragment of an antigen which is capable of stimulating an immune response, preferably a cellular response against the antigen or cells characterized by expression of the antigen and preferably by presentation of the antigen such as diseased cells, in particular cancer cells. Preferably, an antigen peptide is capable of stimulating a cellular response against a cell characterized by presentation of an antigen with class I MHC and preferably is capable of stimulating an antigen-responsive cytotoxic T-lymphocyte (CTL). The antigen peptides according to embodiments are MHC class I and/or class II presented peptides or can be processed to produce MHC class I and/or class II presented peptides. In some embodiments, the antigen peptides comprise an amino acid sequence substantially corresponding to the amino acid sequence of a fragment of an antigen. In some embodiments, said fragment of an antigen is an MHC class I and/or class II presented peptide. In some embodiments, an antigen peptide comprises an amino acid sequence substantially corresponding to the amino acid sequence of such fragment and is processed to produce such fragment, i.e., an MHC class I and/or class II presented peptide derived from an antigen. According to some embodiments, if a peptide is to be presented directly, i.e., without processing, in particular without cleavage, the peptide has a length which is suitable for binding to an MHC molecule, in particular a class I MHC molecule.

In some embodiments, the peptide has a length of 7-20 amino acids, 7-12 amino acids, 8-11 amino acids, for example 7, 8, 9, 10, 11, 12, 13, 14, 15, 16, 17, 18, 19, 20 amino acids in length. As used herein, “cellular immune response”, a “cellular response”, a “cellular response against an antigen” or a similar term are meant to include a cellular response directed to cells characterized by presentation of an antigen with class I or class II MHC. The cellular response relates to cells called T cells or T-lymphocytes which act as either “helper cells” or “killer cells”. The helper T cells (also termed CD4+ T cells) play a central role by regulating the immune response and the killer cells (also termed cytotoxic T cells, cytolytic T cells, CD8+ T cells or CTLS) kill diseased cells such as cancer cells, preventing the production of more diseased cells. In some embodiments, the present disclosure involves the stimulation of an anti-Mycobacterium tuberculosis CTL response against the mycobacterium expressing one or more expressed antigens and preferably presenting such expressed antigens with class I MHC.

As used herein, the term “RNA” refers to a molecule which comprises ribonucleotide residues and preferably being entirely or substantially composed of ribonucleotide residues. As used herein, the term “ribonucleotide” refers to a nucleotide with a hydroxyl group at the 2′-position of a B-D-ribofuranosyl group. As used herein, the term “RNA” comprises double-stranded RNA, single-stranded RNA, isolated RNA such as partially or completely purified RNA, essentially pure RNA, synthetic RNA, and recombinantly generated RNA such as modified RNA which differs from naturally occurring RNA by addition, deletion, substitution and/or alteration of one or more nucleotides. Such alterations can include addition of non-nucleotide material, such as to the end(s) of a RNA or internally, for example at one or more nucleotides of the RNA. Nucleotides in RNA molecules can also comprise non-standard nucleotides, such as non-naturally occurring nucleotides or chemically synthesized nucleotides or deoxynucleotides. These altered RNAs can be referred to as analogs or analogs of naturally occurring RNA.

In some embodiments, the RNA is a mRNA. As used herein, the term “mRNA” means “messenger RNA” and refers to a “transcript” which can be generated by using a DNA template and encodes a peptide or polypeptide. Typically, an mRNA comprises a 5′-UTR, a protein coding region, and a 3′-UTR. mRNA only possesses limited half-life in cells and in vitro. In the context of aspects of the present disclosure, mRNA may be generated by in vitro transcription from a DNA template. As used herein, the term “modification” in the context of the RNA used in aspects of the disclosure includes any modification of an RNA which is not naturally present in said RNA. According to some embodiments, the RNA does not have uncapped 5′-triphosphates. Removal of such uncapped 5′-triphosphates can be achieved by treating RNA with a phosphatase. The RNA according to aspects of the disclosure may have modified ribonucleotides in order to increase its stability and/or decrease cytotoxicity. For example, in some embodiment, 5-methylcytidine in the RNA is substituted partially or completely, for cytidine. In some embodiments, 5-methylcytidine in the RNA is substituted completely for cytidine. Alternatively or additionally, in some embodiments, pseudouridine in the RNA used is substituted partially or completely, for uridine. In some embodiments, pseudouridine in the RNA used is substituted completely for uridine.

In some embodiments, the RNA can be provided with a 5-cap or 5′-cap analog. The term “5-cap” refers to a cap structure found on the 5′-end of an mRNA molecule and generally consists of a guanosine nucleotide connected to the mRNA via an unusual 5′ to 5 triphosphate linkage. In some embodiments, this guanosine is methylated at the 7-position. The term “conventional 5′-cap” refers to a naturally occurring RNA 5′-cap, for example to the 7-methylguanosine cap (m′G). In some embodiments, the 5′-cap includes a 5′-cap analog that resembles the RNA cap structure and is modified to possess the ability to stabilize RNA and/or enhance translation of RNA if attached thereto, preferably in vivo and/or in a cell.

“Parenteral” as used herein in the context of administration means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

The phrases “parenteral administration” and “administered parenterally” as used herein refer to modes of administration other than enteral (i.e., via the digestive tract) and topical administration, usually by injection or infusion, and includes, without limitation, intravenous, intramuscular, intraarterial, intrathecal, intracapsular, intraorbital, intracardiac, intradermal, intraperitoneal, transtracheal, subcutaneous, subcuticular, intraarticular, inhalation, subcapsular, subarachnoid, respiratory mucosal, intraspinal, epidural and intrasternal injection and infusion. Intravenous injection and infusion are often (but not exclusively) used for liposomal drug administration.

As used herein, the term “compound”, “drug” and “active agent” are used interchangeably.

The term “effective amount” as used herein with respect to a compound or the composition means the amount of active compound (also referred herein as active agent or drug) sufficient to cause a bactericidal or bacteriostatic effect. In some embodiments, the effective amount is a “therapeutically effective amount” meaning the amount of active compound that is sufficient alleviate the symptoms of the bacterial infection being treated.

The term “subject” (or, alternatively, “patient”) as used herein refers to an animal, preferably a mammal, most preferably a human that receives either prophylactic or therapeutic treatment.

The term “administration” or “administering” as used herein includes all means of introducing the compounds or the pharmaceutical compositions to the subject in need thereof, including but not limited to, oral, intravenous, intramuscular, intraperitoneal, subcutaneous, transdermal, inhalation, buccal, ocular, sublingual, vaginal, rectal and the like. Administration of the compound or the composition is suitably parenteral. For example, the compounds or the composition can be preferentially administered intravenously, but can also be administered intraperitoneally or via inhalation like is currently used in the clinic for liposomal amikacin in the treatment of Mycobacterium avium (see Shirley et al., Amikacin Liposome Inhalation Suspension: A Review in Mycobacterium avium Complex Lung Disease. Drugs. 2019 April; 79(5):555-562)

The terms “treat,” “treating,” and “treatment,” as used herein, refer to therapeutic or preventative measures such as those described herein.

The term “pharmaceutically acceptable salt” refers to a relatively non-toxic, inorganic or organic acid addition salt of a compound of the present disclosure which salt possesses the desired pharmacological activity.

The term “alkyl” means saturated carbon chains having from one to twenty carbon atoms which may be linear or branched or combinations thereof, unless the carbon chain is defined otherwise. Examples of alkyl groups include methyl, ethyl, propyl, isopropyl, butyl, sec- and tert-butyl, pentyl, hexyl, heptyl, octyl, and the like. Unless stated otherwise specifically in the specification, an alkyl group is optionally substituted.

The term “phosphatidylserine”, with any of it's acyl chain compositions, refers to the L-isomer of serine in the headgroup unless specified in a particular example.

The term “lipid conjugate” refers to a conjugated lipid that inhibits aggregation of lipid particles. Such lipid conjugates include, but are not limited to, polysarcosine (see e.g. WO2021191265A1 which is herein incorporated by reference in its entirety for all purposes), polyamide oligomers (e.g., ATTA-lipid conjugates), PEG-lipid conjugates, such as PEG coupled to dialkyloxypropyls, PEG coupled to diacylglycerols, PEG coupled to cholesterol, PEG coupled to phosphatidylethanolamines, PEG conjugated to ceramides (see, e.g., U.S. Pat. No. 5,885,613, the disclosure of which is herein incorporated by reference in its entirety for all purposes), cationic PEG lipids, and mixtures thereof. PEG can be conjugated directly to the lipid or may be linked to the lipid via a linker moiety. Any linker moiety suitable for coupling the PEG to a lipid can be used including, e.g., non-ester containing linker moieties and ester-containing linker moieties. In preferred embodiments, non-ester containing linker moieties are used.

The abbreviations for the ionizable cationic lipids may be truncated in the Examples from that used in the Tables. For example, AKG-UO-1 may be referred to as UO1:

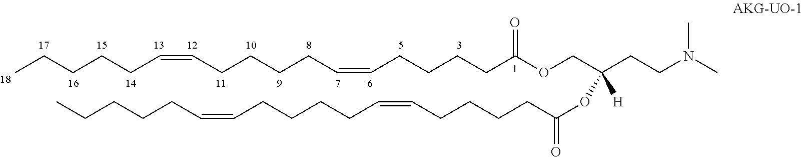 [SMILES: CCCCC/C=C\CCCC/C=C\CCCCC(=O)OC[C@H](CCN(C)C)OC(=O)CCCC/C=C\CCCC/C=C\CCCCC]

For example, AKG-KC2-01 may be referred to as KC2-01.

The abbreviation UT used in various studies refers to untreated samples.

The term “lipidic nanoparticle”, or “LNP”, refers to particles having a diameter of from about 5 to 500 nm. In some embodiments, the lipid nanoparticle comprises one or more active agents. In some embodiments, the lipid nanoparticle comprises a nucleic acid. In some embodiments, the nucleic acid is condensed in the interior of the nanoparticle with a cationic lipid, polymer, or polyvalent small molecule and an external lipid coat that interacts with the biological milieu. Due to the repulsive forces between phosphate groups, nucleic acids are naturally stiff polymers and prefer elongated configurations. In the cell, to cope with volume constraints DNA can pack itself in the appropriate solution conditions with the help of ions and other molecules. Usually, DNA condensation is defined as the collapse of extended DNA chains into compact, orderly particles containing only one or a few molecules. By binding to phosphate groups, cationic lipidic can condense DNA by neutralizing the phosphate charges and allow close packing.

In some embodiments, the active agent is encapsulated into the LNP. In some embodiments, the active agent can be an anionic compounds, for example, but not limited to DNA, RNA, natural and synthetic oligonucleotides (including antisense oligonucleotides, interfering RNA and small interfering RNA), nucleoprotein, peptide, nucleic acid, ribozyme, DNA-containing nucleoprotein, such as an intact or partially deproteinated viral particles (virions), oligomeric and polymeric anionic compounds other than DNA (for example, acid polysaccharides and glycoproteins)). In some embodiments, the active agent can be intermixed with an adjuvant.

In a LNP vaccine product, the active agent is generally contained in the interior of the LNP. In some embodiments, the active agent comprises a nucleic acid. Typically, water soluble nucleic acids are condensed with cationic lipids or polycationic polymers in the interior of the particle and the surface of the particle is enriched in neutral lipids or PEG-lipid derivatives. Additional ionizable cationic lipid may also be at the surface and respond to acidification in the environment by becoming positively charged, facilitating endosomal escape.

Ionizable lipids can have different properties or functions with respect to LNPs. Due to the pKa of the amino group, the lipid molecules can become positively charged in acidic conditions. Under these conditions, lipid molecules can electrostatically bind to the phosphate groups of the nucleic acid which allows the formation of LNPs and the entrapment of the nucleic acid. In some embodiments, the pKa can be low enough that it renders the LNP substantially neutral in surface charge in biological fluids, such as blood, which are at physiological pH values. High LNP surface charge is associated with toxicity, rapid clearance from the circulation by the fixed and free macrophages, hemolytic toxicities, including immune activation (Filion et al Biochim Biophys Acta. 1997 Oct. 23; 1329(2):345-56).

In some embodiments, pKa can be high enough that the ionizable cationic lipid can adopt a positively charged form at acidic endosomal pH values. This way, the cationic lipids can combine with endogenous endosomal anionic lipids to promote membrane lytic nonbilayer structures such as the hexagonal HII phase, resulting in more efficient intracellular delivery. In some embodiments, the pKa ranges between 6.2-7.5. For example, the pKa can be about 6.2, about 6.3, about 6.4, about 6.5, about 6.6, about 6.7, about 6.8, about 6.9, about 7, about 7.1, about 7.2, about 7.3, about 7.4 or about 7.5. Unsaturated tails also contribute to the lipids' ability to adopt nonbilayer structures. (Jayaraman et al., Angew Chem Int Ed Engl. 2012 Aug. 20; 51(34):8529-33).

Release of nucleic acids from LNP formulations, among other characteristics such as liposomal clearance and circulation half-life, can be modified by the presence of polyethylene glycol and/or sterols (e.g. cholesterol) or other potential additives in the LNP, as well as the overall chemical structure, including pKa of any ionizable cationic lipid included as part of the formulation.

The terms “encapsulation” and “entrapped,” as used herein, refer to the incorporation or association of the mRNA, DNA, siRNA or other nucleic acid pharmaceutical agent in or with a lipidic nanoparticle. As used herein, the term “encapsulated” refers to complete encapsulation or partial encapsulation. A siRNA may be capable of selectively knocking down or down regulating expression of a gene of interest. For example, an siRNA could be selected to silence a gene associated with a particular disease, disorder, or condition upon administration to a subject in need thereof of a nanoparticle composition including the siRNA. A siRNA may comprise a sequence that is complementary to an mRNA sequence that encodes a gene or protein of interest.

With regard to a small molecule TLR agonist, the term “encapsulation” includes encapsulation of the agonist into the LNP, incorporation of the agonist into the bilayer structure of the LNP, and/or attachment of the agonist moiety to the surface of the LNP, including attachment via a spacer group, such as a hydrophilic polymer chain.

The term “mol %” of a lipid component in the LNP composition refers to the molar percent of the lipid component relative to the sum of molar amounts of all lipid components in the nanoparticle (total lipid). The term “N/P” or “N/P ratio” of a nanoparticle comprising a nucleic acid and a cationic lipid (CL) is the ratio between the total number of cationically charged nitrogen atoms of the cationic lipid and the total number of anionically charged phosphate groups of the nucleic acid in the nanoparticle composition. The term “N/P” or “N/P ratio” of a nanoparticle comprising a nucleic acid and an ionizable cationic lipid (ICL) refers to the ratio between the total number of nitrogen atoms of the cationic lipid ionizable to cationic charge and the total number of anionically charged phosphate groups of the nucleic acid in the nanoparticle composition. The N/P ratio is calculated as the amount of ICL expressed in gram-equivalents of the ionizable nitrogen groups divided by the amount of the nucleic acid expressed in gram-equivalents of the nucleic acid phosphate groups. For calculations of N/P ratios an average equivalent weight of an RNA nucleotide (corresponding to one phosphate group) was assumed to be 330 Da.

As used herein, the term “pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

The term “peptide,” “polypeptide” and “protein” are used interchangeably to denote a sequence polymer of at least two amino acids covalently linked by an amide bond (also referred herein as peptide bond).

“Identity,” as known in the art, is a relationship between two or more polypeptide or protein sequences, or nucleic acid sequences as determined by comparing the sequences. In the art, “identity” also refers to the degree of sequence relatedness between polypeptides or proteins, as determined by the match between strings of such sequences. “Identity” can be readily calculated by any bioinformational methods known in the art. “Percent (%) identity” is defined as the percentage of residues (amino acid residues or nucleic acid residues) in the candidate amino acid or nucleic acid sequence that are identical with the residues in the amino acid sequence or nucleic acid sequence of a second sequence after aligning the sequences and introducing gaps, if necessary, to achieve the maximum percent identity.

The term “substantial identity” or “substantial similarity,” as used herein, when referring to a nucleic acid or fragment thereof, indicates that when optimally aligned with appropriate nucleotide insertions or deletions with another nucleic acid (or its complementary strand), there is nucleotide sequence identity in at least about 95% to 99% of the sequence. The term “substantial identity” or “substantial similarity,” as used herein, when referring to a protein or fragment thereof, indicates that when optimally aligned there is an amino acid sequence identity in at least about 95% to 99% of the sequence.

Various aspects and embodiments are described in further detail in the following subsections.

### (II) LIPOSOMAL COMPOSITIONS

Liposomal nanoparticle (LNP) compositions can comprise an ionizable lipid, a sterol, and one or more phospholipids. In some embodiments, the LNP compositions further comprise a nucleic acid such as mRNA or DNA for administration in a pharmaceutical composition such as a vaccine. In some embodiments, the LNP compositions optionally further comprise a conjugated lipid. Other aspects relate to compositions comprising lipidic nanoparticles comprising ionizable cationic lipid, the lipidic nanoparticles containing nucleic acids. In some embodiments, nucleic acids are encapsulated into the lipidic nanoparticles.

In some embodiments, a lipid nanoparticle (LNP) composition comprises an ionizable cationic lipid, sterol, one or more phospholipids comprising at least one anionic phospholipid, a conjugated lipid and one or more nucleic acid sequences agonizing TLR9 or TLR 7/8 combined with antigens recognized by T cells. In some embodiments, a lipid nanoparticle (LNP) composition comprises an ionizable cationic lipid, sterol, one or more phospholipids comprising at least one anionic phospholipid, a conjugated lipid and one or more nucleic acid sequences agonizing TLR9 or TLR 7/8 combined with Mycobacterium tuberculosis antigens recognized by T cells. In some aspects, the nucleic acid sequence encodes a polypeptide that is recognized by T cells. Peptide fragments can be generated from an antigen that are recognized by T cell receptors.

In some embodiments, a lipid nanoparticle (LNP) composition comprises an ionizable cationic lipid, sterol, one or more phospholipids comprising at least one anionic phospholipid, a conjugated lipid and one or more nucleic acid sequence encoding a T cell epitope from Mycobacterium tuberculosis (Mtb). In some embodiments, a lipid nanoparticle (LNP) composition comprises an ionizable cationic lipid, sterol, one or more phospholipids comprising at least one anionic phospholipid, a conjugated lipid and one or more nucleic acid sequence encoding Mycobacterium tuberculosis antigens recognized by T cells.

Aspects of the disclosure provide for improved compositions of ionizable lipid nanoparticles for the delivery of therapeutic nucleic acids to cells. Anionic phospholipids, including phosphatidylserine and phosphatidylglycerol are included in the lipid nanoparticles to increase the transfection efficiency in dendritic cells. The further incorporation of ionizable lipids in an LNP formulation with gem di-substitution of mono-unsaturated alkyl chains (single olefin) on 2-position of 1,3-dioxolane or ketal demonstrated high levels of transfection in human dendritic cells, compared to other ionizable lipids in the same family, and demonstrated good stability to oxidative damage.

### (A) TLR-activating Nucleic Acids and Small Molecules

Compositions comprising TLR-activating compositions are provided. In some embodiments, a lipid nanoparticle (LNP) composition comprises one or more TLR-activating nucleic acids and/or small molecule(s), in a LNP composition comprising an ionizable cationic lipid, sterol, one or more phospholipids comprising at least one anionic phospholipid, and a conjugated lipid.

In some embodiments, the TLR-activating composition is a nucleic acid. As used herein, a “nucleic acid” refers to a deoxyribonucleic acid (DNA) or ribonucleic acid (RNA). In some embodiments, the nucleic acid is an RNA, for example an in vitro transcribed RNA (IVT RNA) or synthetic RNA. Nucleic acids include according to aspects of the disclosure genomic DNA, cDNA, mRNA, recombinantly produced and chemically synthesized molecules. According to aspects of the disclosure, a nucleic acid may be present as a single-stranded or double-stranded and linear or covalently circularly closed molecule. A nucleic acid can, according to aspects of the disclosure, be isolated. In some embodiments, the nucleic acid (i) was amplified in vitro, for example via polymerase chain reaction (PCR), (ii) was produced recombinantly by cloning, (iii) was purified, for example, by cleavage and separation by gel electrophoresis, or (iv) was synthesized, for example, by chemical synthesis. A nucleic can be employed for introduction into, i.e. transfection of cells, in particular, in the form of RNA which can be prepared by in vitro transcription from a DNA template. The RNA can moreover be modified before application by stabilizing sequences, capping, and polyadenylation. In some embodiments, some or all uridine nucleosides in the TLR-activating mRNA are substituted with N1-methyl-pseudouridine.

According to aspects of the disclosure, the stability and translation efficiency of RNA may be modified as required. For example, RNA may be stabilized and its translation increased by one or more modifications having a stabilizing effects and/or increasing translation efficiency of RNA. Such modifications are described, for example, in PCT/EP2006/009448 incorporated herein by reference in its entirety. In order to increase expression of the RNA used according to aspects of the present disclosure, it may be modified within the coding region, i.e. the sequence encoding the expressed peptide or protein, preferably without altering the sequence of the expressed peptide or protein, so as to increase the GC content to increase mRNA stability and to perform a codon optimization and, thus, enhance translation in cells.

In some embodiments, the ionizable lipid encapsulate the nucleic acid in a LNP formulation. In some embodiments, the nucleic acid is a mRNA molecule. In some embodiments, the nucleic acid is entrapped in the lipidic nanoparticle with a compound disclosed herein, including compounds of disclosed herein or combinations thereof, wherein the nucleic acid is either RNA or DNA. In some embodiments, the nucleic acid is mRNA. In some embodiments, the nucleic acid is siRNA. In some embodiments, the nucleic acid is DNA.

In some embodiments, the TLR-activating composition is a nucleic acid. In some embodiments, the TLR-activating nucleic acid is RNA oligonucleotide. In some embodiments, the TLR-activating nucleic acid is a DNA oligonucleotide. In some embodiments, the TLR-activating nucleic acid comprises more than one nucleic acid. Provided in some aspect of the disclosure is a TLR-activating nucleic acid lipid nanoparticle (LNP) composition comprising: a nucleic acid having at least 90% identity (e.g. 90%, 91%, 92%, 93%, 94%, 95%, 96%, 97%, 98%, 99% or more) with a nucleic acid sequence set forth herein.

In some embodiments, the nucleic acid is a TLR9-activating oligonucleotide, CPG1018. In some embodiments, one of the nucleotides is a DNA TLR9 agonist oligonucleotide and/or one of the nucleic acids is a TLR7/8 RNA oligonucleotide agonist. In some embodiments, a lipid nanoparticle (LNP) composition comprises an ionizable cationic lipid, sterol, one or more phospholipids comprising at least one anionic phospholipid, a conjugated lipid and one or more nucleic acid sequences activating TLR 9 selected from the group consisting of: SEQ ID NO:2, SEQ ID NO:3, SEQ ID NO:6, SEQ ID NO:7 and SEQ ID NO:8.

In some embodiments, a lipid nanoparticle (LNP) composition comprises a nucleic acid sequence comprising one or more nucleic acid sequences activating TLR 7 or 8 selected from the group consisting of: SEQ ID NO: 11, SEQ ID NO:25, SEQ ID NO: 113, SEQ ID NO: 114, SEQ ID NO: 116 and SEQ ID NO:212, an ionizable cationic lipid, sterol, one or more phospholipids comprising at least one anionic phospholipid, and a conjugated lipid. In some embodiments, a lipid nanoparticle (LNP) composition comprises an ionizable cationic lipid, sterol, one or more phospholipids comprising at least one anionic phospholipid, a conjugated lipid and one or more nucleic acid sequences activating RIG-I. In some embodiments, a lipid nanoparticle (LNP) composition comprises an ionizable cationic lipid, sterol, one or more phospholipids comprising at least one anionic phospholipid, a conjugated lipid and a nucleic acid sequence comprising one or more nucleic acid sequences selected from the group consisting of: 3P-GFP2 (SEQ ID NO. 127) annealed with either SEQ ID NO. 128 or SEQ ID NO. 129, 3P-A24 (SEQ ID NO. 130) annealed with either SEQ ID NO. 133 or SEQ ID NO. 134, 3P-G24 (SEQ ID NO. 130) annealed with either SEQ ID NO. 140 or SEQ ID NO. 141, 3P-C24 (SEQ ID NO. 130) annealed with either SEQ ID NO. 147 or SEQ ID NO. 148 and 3P-U24 (SEQ ID NO. 151) annealed with either SEQ ID NO. 154 or SEQ ID NO. 155.

In some embodiments, the LNP composition has a N/P ratio of 4 to 7. In some embodiments, the LNP composition has a N/P ratio of 5 to 6. In some embodiments, the composition has a N/P ratio of 5.3. In some embodiments, the N/P ratio is 3. In some embodiments, the N/P ratio is 7. In some embodiments, the TLR-activating nucleic acid is a RNA oligonucleotide TLR 7 and/or 8 agonist, and the N/P ratio is between 3 and 8.

In some embodiments, the LNP composition further comprises an adjuvant. In some embodiments, the LNP comprises a small molecule agonist adjuvant such as compound that activates RIG-I as an adjuvant, such as KIN1148 or a pharmaceutically acceptable salt thereof (See, e.g., Hemann et al., 2023, J Immunol. 210:1247-1256).

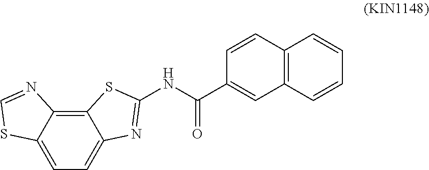 [SMILES: O=C(Nc1nc2ccc3scnc3c2s1)c1ccc2ccccc2c1]

In some embodiments, the TLR agonist comprised in the LNP composition is a small molecule compound. As used herein, a “small molecule compound” is a compound with a molecular weight between 0.1 and 2.0 KDa, typically between 0.1 and 1.0 KDa, and lacking a motif of multiple repetitive units characteristic of a polymer. A number of small molecule TLR agonists are known in the art. They include, without limitation, TLR4 agonists (Romerio and Peri, 2020, Front. Immunol. 11:1210), and TLR7/8 agonists (Bhagchandani et al. 2021, Adv Drug Delivery Rev. 175:113803). Another class of small molecule agonists are agonists of retinoid acid-inducible gene I (RIG-I)-like receptors (RLR), such as KIN1148 (Hemann et al, 2023, J Immunol. 210:1247-1256).

In some embodiments, the composition further comprises an additional TLR4 agonist. In some embodiments, the additional TLR4 agonist is chosen from Monophosphoryl Lipid A (MPL), Monophosphoryl Lipid A-504, Monophosphoryl 3-Deacyl Lipid A, and Monophosphoryl Hexa-acyl Lipid A, 3-Deacyl.

Other aspects of the disclosure relate to lipid nanoparticles or targeted lipid nanoparticles that incorporate mRNA coding for major histocompatibility complex class I (MHC-I) or class II (MHC-II) epitopes. In some embodiments, mRNAs coding for MHC-I and MHC-II epitopes are incorporated into a single LNP vaccine. In some embodiments, the epitopes are enriched for those present in Mycobacterium tuberculosis when compared to BCG or nontuberculosis mycobacterium (NTM). In some embodiments, the epitopes in the mRNA cassette are linked with nonimmunogenic linkers. In other embodiments the junctions between epitopes have been optimized to reduce the propensity for forming neoepitopes.

### (B) Ionizable Cationic Lipids

In an embodiment, the LNP comprises an ionizable cationic lipid. As used herein “ionizable cationic lipid”, “ionizable lipid” and “ICL” are used interchangeably. An ICL is a lipid that comprises an ionizable moiety capable of bearing a charge (e.g., a positive charge e.g., a cationic lipid) under certain conditions (e.g., at a certain pH range, e.g., under physiological conditions). The ionizable moiety may comprise an amine, and preferably a substituted amine. An ionizable lipid may be a cationic lipid or an anionic lipid. In addition to an ionizable moiety, an ionizable lipid may contain an alkyl or alkenyl group, e.g., greater than six carbon atoms in length (e.g., greater than about 8 carbons, 10 carbons, 12 carbons, 14 carbons, 16 carbons, 18 carbons, 20 carbons or more in length). Additional ionizable lipids that may be included in an LNP described herein are disclosed in Jayaraman et al. (Angew. Chem. Int. Ed. 51:8529-8533 (2012)), and Semple et al. Nature Biotechnol. 28:172-176 (2010)), each of which is incorporated herein by reference in its entirety.

In some embodiments, the ionizable cationic lipids are cationic (i.e. positively charged) at acidic pH, such as encountered intracellularly following endocytosis or phagocytosis by a cell. The same lipids, and compositions containing them, are near neutral in charge when present at pH 7.4. These lipids may also have a single olefin group present in their alkyl or acyl groups. In some embodiments, the lipidic nanoparticles are in an aqueous medium.

In some embodiments, ionizable cationic lipids (ICLs) are provided. In some embodiments, cationic lipids are engineered with improved stability to oxidative degradation while in storage, while retaining high transfection activity or potency in cells. Aspects of the disclosure are based in part on the discovery that LNP compositions comprising mRNA and certain ionizable cationic lipids (ICL) enhanced expression of the mRNA in human dendritic cells.

In some embodiments, ionizable cationic lipid compositions useful in the preparation of liposomal nanoparticle (LNP) compositions are provided.

In some embodiments, a lipid nanoparticle (LNP) composition comprises an ionizable lipid having a chemical structure consisting of a pair of linear polyunsaturated lipid tails covalently bound to a head group, the head group comprising a dialkyl amino group; the head group comprising a heterocyclyl or alkyl portion covalently bound to the dialkyl amino group and optionally further comprising a phosphate group; and each polyunsaturated lipid tail being unsaturated except for at least two olefins separated by at least two methylene groups along the length of the lipid tail, and optionally comprising a single acyl group at the end of the lipid tail covalently bound to the head group. In some aspects, each lipid tail in the ionizable lipid is identical, and each lipid tail has a total of two olefins separated only by an unsubstituted ethylene, n-propyl, or n-butyl. In some embodiments, each lipid tail of the ioniziable lipid further comprises an acyl group joined to an oxygen of the headgroup to form an ester, and has a total of 16 or 18 carbon atoms including the acyl group.

In some embodiments, liposomal compositions are provided comprising an ionizable cationic lipid having (a) a pair of linear C₁₆ or C₁₈ hydrocarbon chains each comprising a single unsaturated alkenyl double bond within each polyene hydrocarbon chain, covalently bound to a head group comprising a dialkyl amino alkyl group. In some embodiments, the head group of the ionizable cationic lipid has a dialkyl amino group having a pKa of about 6.3-7.5. In some embodiments, the head group of the ionizable cationic lipid comprises a heterocyclyl or alkyl portion covalently bound to the dialkyl amino group. In some embodiments, the head group of the ionizable cationic lipid optionally further comprises a phosphate group. In some embodiments, each lipid tail of the ionizable cationic lipid compound is identical, and each lipid tail has a total of one olefin with a total length of 15, 16, 17 or 18 carbons.

In some embodiments, the nucleic acid is entrapped in the lipidic nanoparticle with a compound disclosed herein, including compounds of Formula (I), (I-A), (II), (III) or (IV-A) combinations thereof.

In some embodiments, a lipid nanoparticle (LNP) composition comprises an ionizable cationic lipid having the chemical structure of Formula (I),

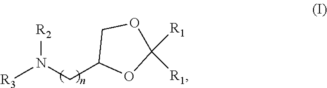

wherein the total length of the R₁ hydrocarbon chain is C₁₅-C₁₈; R₂ and R₃ are each independently (C₁-C₄) alkyl optionally substituted with hydroxyl; and n is an integer equal to 2, 3 or 4. In some embodiments, the total length of the R₁ hydrocarbon chain in Formula (I) is C₁₆-C₁₈. In some embodiments, the total length of the R₁ hydrocarbon chain is C₁₆ or C₁₈.

In some embodiments, a lipid nanoparticle (LNP) composition comprises an ionizable cationic lipid having the chemical structure of Formula (I):

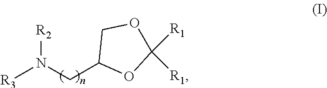

wherein
   R₁ is

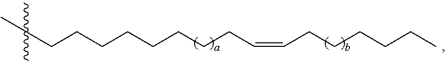

wherein a is 0 or 1; b is 1, 2, 3 or 4, provided the sum a+b is 1, 2, 3 or 4;
R₂ and R₃ are each independently (C₁-C₄) alkyl optionally substituted with hydroxyl; and
n is an integer equal to 2, 3 or 4.

In some embodiments, ionizable cationic lipid compositions are provided. In some embodiments, a lipid nanoparticle (LNP) composition comprises an ionizable cationic lipid having the chemical structure of Formula (I), wherein a is 0 or 1; b is 1, 2, 3 or 4, provided the sum a+b is 1, 2, 3 or 4; R₂ and R₃ are each methyl; and n is an integer equal to 3. In some embodiments, n is 2 or 3. In some embodiments, a is 0. In some embodiments, b is 1, 2 or 3. In some embodiments, a is 1. In some embodiments, b is 1, 2 or 3. In some embodiments, R₂ and R₃ are each methyl. In some embodiments, each R₁ is independently

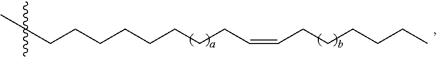

a is 0 or 1 and b is 1 or 3; R₂ and R₃ are each methyl; and n is 2 or 3. In some embodiments, n is 3. In some embodiments, a lipid nanoparticle (LNP) composition comprises an ionizable cationic lipid having the chemical structure of Formula (I), wherein R₂ and R₃ are each independently methyl; and n is an integer equal to 2 or 3. In some embodiments, a is 0. In some embodiments, b is 1. In some embodiments, b is 3. In some embodiments, a is 1. In some embodiments, b is 1. In some embodiments, b is 3. In some embodiments, n is 2. In some embodiments, n is 3.

In some embodiments, a lipid nanoparticle (LNP) composition comprises an ionizable cationic lipid having the chemical structure of Formula (I-A):

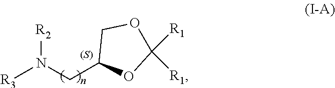

wherein each R₁ is a hydrocarbon chain having a total length of C₁₅-C₁₈ and optionally comprising one or more olefin and R₂ and R₃ are each independently (C₁-C₄) alkyl optionally substituted with hydroxyl; and n is an integer equal to 2, 3 or 4.

In some embodiments, a lipid nanoparticle (LNP) composition comprises an ionizable cationic lipid having the chemical structure of Formula (I-A):

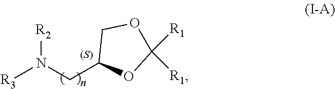

wherein
   R₁ is,

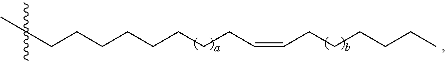

wherein a is 0 or 1; b is 1, 2, 3 or 4, provided the sum a+b is 1, 2, 3 or 4;
R₂ and R₃ are each methyl; and
n is an integer equal to 3.

In some embodiments, a and b of the two R₁ hydrocarbon chains are the same. In some embodiments, a and b of the two R₁ hydrocarbon chains are different. In some embodiments, one of the two R₁ hydrocarbon chains is a saturated C₁₂-C₁₈ alkyl.

In some embodiments, a lipid nanoparticle (LNP) composition comprises an ionizable cationic lipid having the chemical structure of Formula (I-A), wherein R₁ is a saturated C15-C18 hydrocarbon chain, R₂ and R₃ are each methyl; and n is an integer equal to 3. In some embodiments, a and b of the two R₁ hydrocarbon chains are the same. In some embodiments, a and b of the two R₁ hydrocarbon chains are different. In some embodiments, one of the two R₁ hydrocarbon chains is a saturated C₁₂-C₁₈ alkyl. In some embodiments, a lipid nanoparticle (LNP) composition comprises an ionizable cationic lipid having the chemical structure of Formula (I-A), wherein R₂ and R₃ are each independently (C₁-C₄) alkyl optionally substituted with hydroxyl; and n is an integer equal to 2, 3 or 4. In some embodiments, a lipid nanoparticle (LNP) composition comprises an ionizable cationic lipid having the chemical structure of Formula (I-A), wherein one of the two R₁ hydrocarbon chains is a saturated C₁₂-C₁₈ alkyl.

In some embodiments, a lipid nanoparticle (LNP) composition comprises an ionizable cationic lipid having the chemical structure of Formula (I) or (IA), wherein a is 0 and b is 1, 2, 3 or 4. In some embodiments, a lipid nanoparticle (LNP) composition comprises an ionizable cationic lipid having the chemical structure of Formula (I), wherein a is 0 and b is 1 or 3. In some embodiments, a lipid nanoparticle (LNP) composition comprises an ionizable cationic lipid having the chemical structure of Formula (I), wherein a is 0 and b is 1. In some embodiments, a lipid nanoparticle (LNP) composition comprises an ionizable cationic lipid having the chemical structure of Formula (I), wherein a is 0 and b is 3.

In some embodiments, a lipid nanoparticle (LNP) composition comprises an ionizable cationic lipid having the chemical structure of Formula (I), wherein a is 1 and b is 1, 2, 3 or 4. In some embodiments, a lipid nanoparticle (LNP) composition comprises an ionizable cationic lipid having the chemical structure of Formula (I), wherein a is 1 and b is 1 or 3. In some embodiments, a lipid nanoparticle (LNP) composition comprises an ionizable cationic lipid having the chemical structure of Formula (I), wherein a is 1 and b is 1. In some embodiments, a lipid nanoparticle (LNP) composition comprises an ionizable cationic lipid having the chemical structure of Formula (I), wherein a is 1 and b is 3.

In some embodiments, a lipid nanoparticle (LNP) composition comprises an ionizable cationic lipid having the chemical structure of Formula (I), wherein a is 0 or 1; b is 1, 2, 3 or 4, provided the sum a+b is 1, 2, 3 or 4; R₂ and R₃ are each methyl; and n is an integer equal to 2, 3 or 4. In some embodiments, a lipid nanoparticle (LNP) composition comprises an ionizable cationic lipid having the chemical structure of Formula (I), wherein a is 0 or 1; b is 1, 2, 3 or 4, provided the sum a+b is 1, 2, 3 or 4; R₂ and R₃ are each methyl; and n is an integer equal to 2 or 3. In some embodiments, a lipid nanoparticle (LNP) composition comprises an ionizable cationic lipid having the chemical structure of Formula (I), wherein a is 0 or 1; b is 1, 2, 3 or 4, provided the sum a+b is 1, 2, 3 or 4; R₂ and R₃ are each methyl; and n is an integer equal to 2. n some embodiments, a lipid nanoparticle (LNP) composition comprises an ionizable cationic lipid having the chemical structure of Formula (I), wherein a is 0 or 1; b is 1, 2, 3 or 4, provided the sum a+b is 1, 2, 3 or 4; R₂ and R₃ are each methyl; and n is an integer equal to 3.

In some embodiments, the LNP compositions comprises a KC3 ionizable cationic lipid, a KC4 ionizable cationic lipid or a mixture of a KC3 ionizable cationic lipid and a KC4 ionizable cationic lipid.

In some embodiments, the LNP compositions comprises a KC3 ionizable cationic lipid.

Unless otherwise indicated, the term “KC3 ionizable cationic lipid” as used herein refers to an ionizable cationic lipid having the chemical formula:

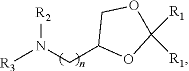

wherein each R₁ is the same or different and is a linear C₁₅ to C₁₉ hydrocarbon chain each comprising one or more unsaturated alkenyl double bond within each polyene hydrocarbon chain; R₂ and R₃ are each independently methyl; and n is 3. In some aspects, each R₁ in the KC3 ionizable cationic lipid is the same and is a linear C₁₆ or C₁₈ hydrocarbon chain each comprising a single unsaturated alkenyl double bond within each polyene hydrocarbon chain.

In some embodiments, the LNP compositions comprises a KC4 ionizable cationic lipid. Unless otherwise indicated, the term “KC4 ionizable cationic lipid” as used herein refers to an ionizable cationic lipid having the chemical formula:

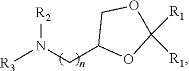

, wherein each R₁ is the same or different and is a linear C₁₅ to C₁₉ hydrocarbon chain each comprising one or more unsaturated alkenyl double bond within each polyene hydrocarbon chain; R₂ and R₃ are each independently methyl; and n is 4. In some aspects, each R₁ in the KC4 ionizable cationic lipid is the same and is a linear C₁₆ or C₁₈ hydrocarbon chain each comprising a single unsaturated alkenyl double bond within each polyene hydrocarbon chain.

In some aspects, each R₁ in the KC3 or a KC4 ionizable cationic lipid is the same or different and is

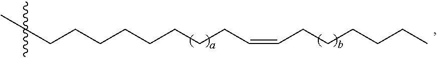

herein a is 0 or 1; b is 1, 2, 3 or 4, provided the sum a+b is 1, 2, 3 or 4. In some aspects, each R₁ in the KC3 or a KC4 ionizable cationic lipid is the same or different and is

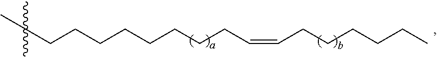

wherein a is 1 and b is 1 or 3.

In some aspects, each R₁ in the KC3 or a KC4 ionizable cationic lipid is the same or different and is

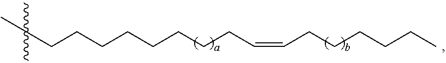

wherein a is 1 and b is 1. In some aspects, each R₁ in the KC3 or a KC4 ionizable cationic lipid is the same or different and is

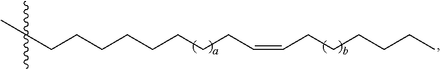

wherein a is 1 and b is 3.

In some aspects, each R₁ in the KC3 or a KC4 ionizable cationic lipid is the same or different and is

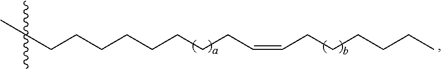

wherein a is 0 and b is 1 or 3. In some aspects, each R₁ in the KC3 or a KC4 ionizable cationic lipid is the same or different and is

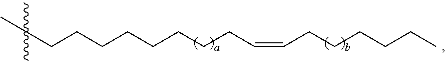

wherein a is 0 and b is 1. In some aspects, each R₁ in the KC3 or a KC4 ionizable cationic lipid is the same or different and is

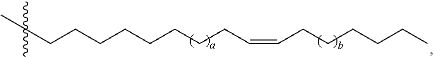

wherein a is 0 and b is 3.

In some aspects, each R₁ in the KC3 or a KC4 ionizable cationic lipid is the same or different and is

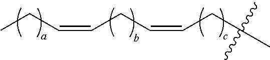

wherein
   a is 1, 2, 3 or 4;
   b is 2, 3 or 4; and
   c is 3, 4, 5, 6, or 7,
provided that the sum of a, b and c is 10, 11, 12 or 13.

In some aspects, each R₁ in the KC3 or a KC4 ionizable cationic lipid is the same or different and is

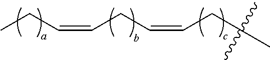

wherein
   a is 1, 2, 3 or 4;
   b is 4; and
   c is 3, 4, 5, 6, or 7,
provided that the sum of a, b and c is 11 or 13.

In some aspects, each R₁ in the KC3 or a KC4 ionizable cationic lipid is the same and is a linear C₁₆ hydrocarbon chain each comprising one unsaturated alkenyl double bond within each polyene hydrocarbon chain. In some aspects, each R₁ in the KC3 or a KC4 ionizable cationic lipid is the same and is a linear C₁₈ hydrocarbon chain each comprising one unsaturated alkenyl double bond within each polyene hydrocarbon chain. In some aspects, each R₁ in the KC3 or a KC4 ionizable cationic lipid is the same and is a linear C₁₆ hydrocarbon chain each comprising two unsaturated alkenyl double bonds within each polyene hydrocarbon chain. In some aspects, each R₁ in the KC3 or a KC4 ionizable cationic lipid is the same and is a linear C₁₆ hydrocarbon chain each comprising two unsaturated alkenyl double bonds within each polyene hydrocarbon chain, wherein the alkenyl double are separated by two or more saturated alkylene groups. In some aspects, each R₁ in the KC3 or a KC4 ionizable cationic lipid is the same and is a linear C₁₈ hydrocarbon chain each comprising one unsaturated alkenyl double bond within each polyene hydrocarbon chain. In some aspects, each R₁ in the KC3 or a KC4 ionizable cationic lipid is the same or different and is a linear C₁₆ or C₁₈ hydrocarbon chain each comprising one or two unsaturated alkenyl double bond within each polyene hydrocarbon chain.

In some embodiments, the LNP compositions comprises a mixture of a KC3 ionizable cationic lipid and a KC4 ionizable cationic lipid. Aspects of the disclosure are based in part on the discovery that selection of certain cationic ionizable lipids can enhance the transfection of human dendritic cells. For example, the KC3 cationic ionic lipids were more active in transfecting human dendritic cells in LNP compositions than either the KC2 or diacyl ionizable lipids (UO series). Among the LNP compositions comprising KC3 ionizable cationic lipids, those LNP compositions with ionizable cationic lipids having monounsaturated alkyl chains were unexpectedly both more active and more stable to oxidative degradation than those containing those with the dilinoleyl alkyl chains.

In some aspects, the ionizable cationic lipid comprises 3-((S)-2,2-di((Z)-octadec-9-en-1-yl)-1,3-dioxolan-4-yl)-N,N-dimethylpropan-1-amine (KC3-OA). In some aspects, the ionizable cationic lipid further comprises a KC4 ionizable cationic lipid, such as 4-rac-2,2-di((Z)-octadec-9-en-1-yl)-1,3-dioxolan-4-yl)-N,N-dimethylbutan-1-amine (AKG-KC4-OA).

In some embodiments, the present disclosure provides compositions comprising ionizable cationic lipids. Aspects of the disclosure include compositions comprising 3-rac-2,2-di((Z)-octadec-9-en-1-yl)-1,3-dioxolan-4-yl)-N,N-dimethylpropan-1-amine (AKG-KC3-OA racemate) or chiral purified forms of the AKG-KC3-OA racemate such as KC3-OA(S) and KC3-OA(R), and methods of making and purifying the same. Aspects of the disclosure include compositions comprising 4-rac-2,2-di((Z)-octadec-9-en-1-yl)-1,3-dioxolan-4-yl)-N,N-dimethylbutan-1-amine (AKG-KC4-OA), and methods of making the same. In some embodiments, a composition comprises an ionizable cationic lipid selected from one or more of the following: (a) a racemic mixture of 3-rac-2,2-di((Z)-octadec-9-en-1-yl)-1,3-dioxolan-4-yl)-N,N-dimethylpropan-1-amine (KC3-OA racemate), or KC3-OA enantiomer; and (b) 4-rac-2,2-di((Z)-octadec-9-en-1-yl)-1,3-dioxolan-4-yl)-N,N-dimethylbutan-1-amine (AKG-KC4-OA racemate). In some embodiments, a composition comprises a mixture of (R) and (S) enantiomers of KC3-OA ionizable cationic lipid, or a mixture of (R) and (S) enantiomers of KC4-OA ionizable cationic lipid. In some embodiments, a composition comprises a mixture of (R) and (S) enantiomers of KC3-OA ionizable cationic lipid, or a mixture of (R) and (S) enantiomers of KC4-OA ionizable cationic lipid, and the mixture is racemic.

Some aspects of the disclosure relate to bioreducible ionizable cationic lipids of Formula (I) or (I-A), wherein each R₁ is independently linear hydrocarbon alkyl chains each comprising a hydrolysable or bioreducible group, such as an ester group (e.g, —O—CO— or —CO—O—); and R₂ and R₃ are each independently (C₁-C₄) alkyl optionally substituted with hydroxyl; and n is an integer equal to 2, 3 or 4.

In some embodiments, a bioreducible or hydrolysable ionizable cationic lipid comprises a compound of Formula (I) or Formula (I-A) wherein each of R₂ and R₃ is methyl and n is 3 or 4 and each R₁ is the same. In some embodiments, a bioreducible or hydrolysable ionizable cationic lipid comprises a compound of Formula (I) or Formula (I-A) wherein each of R₂ and R₃ is methyl and each R₁ is a hydrocarbon chain comprising a single ester group with a total 15-22 atoms, and optionally comprising one or two olefin groups. In some embodiments, a bioreducible or hydrolysable ionizable cationic lipid comprises a compound of Formula (I) or Formula (I-A) wherein each of R₂ and R₃ is methyl and each R₁ is a hydrocarbon chain comprising a single ester group with a total 15-22 atoms, and optionally comprising one olefin group. In some embodiments, a bioreducible or hydrolysable ionizable cationic lipid comprises a compound of Formula (I) or Formula (I-A) wherein each of R₂ and R₃ is methyl and each R₁ is a hydrocarbon chain comprising a single ester group with a total 15-22 atoms, and optionally comprising two olefin groups separated by at least two methylene groups.

In some embodiments, a bioreducible or hydrolysable ionizable cationic lipid comprises a compound of Formula (I) or Formula (I-A) wherein each of R₂ and R₃ is methyl, n is 3 or 4 and each R₁ comprises an ester joining a proximal saturated C₇₋₈ hydrocarbon and a distal C₁₂₋₁₅ hydrocarbon optionally comprising one or more olefins and one or more methyl substituents. In some embodiments, a bioreducible or hydrolysable ionizable cationic lipid comprises a compound of Formula (I) or Formula (I-A) wherein each of R₂ and R₃ is methyl, n is 3 or 4 and each R₁ comprises an ester joining a proximal saturated C₄₋₁₀ hydrocarbon and a distal C₁₀₋₂₀ hydrocarbon optionally comprising one or more olefins and one or more methyl substituents.

In some embodiments, a bioreducible or hydrolysable ionizable cationic lipid comprises a compound of Formula (I) or Formula (I-A) wherein each of R₂ and R₃ is methyl, n is 3 or 4 and each R₁ comprises a proximal C₇₋₁₀ hydrocarbon and a distal saturated isoprenoid alkane group joined by an ester. In some embodiments, a bioreducible or hydrolysable ionizable cationic lipid comprises a compound of Formula (I) or Formula (I-A) wherein each of R₂ and R₃ is methyl, n is 3 or 4 and each R₁ comprises a proximal C₇₋₁₀ hydrocarbon and a distal saturated phytane group joined by an ester. In some embodiments, a bioreducible or hydrolysable ionizable cationic lipid comprises a compound of Formula (I) or Formula (I-A) wherein each of R₂ and R₃ is methyl, n is 3 or 4 and each R₁ comprises a proximal C₇₋₁₀ hydrocarbon and a distal unsaturated phytane group comprising one or more olefins joined by an ester.

In some embodiments, a bioreducible or hydrolysable ionizable cationic lipid comprises a compound of Formula (I) or Formula (I-A) wherein each of R₂ and R₃ is methyl, n is 3 or 4 and each R₁ is —(CH₂)₇₋₁₀-A-Y, wherein A is —C(═O)—O— or —O—C(═O)—, and Y is —(CH₂—CH₂—CH(—CH₃)—CH₂)₃—H or —(CH₂—CH₂—CH(—CH₃)—CH₂)₃—H or —CH₂—CH₂—(CH₂—CH₂—CH(—CH₃)—CH₂)₂—H. In some embodiments, a bioreducible or hydrolysable ionizable cationic lipid comprises a compound of Formula (I) or Formula (I-A) wherein each of R₂ and R₃ is methyl, n is 3 or 4 and each R₁ is —(CH₂)₇-A-Y, wherein A is —C(═O)—O— or —O—C(═O)—, and Y is —(CH₂—CH₂—CH(—CH₃)—CH₂)₃—H. In some embodiments, a bioreducible or hydrolysable ionizable cationic lipid comprises a compound of Formula (I) or Formula (I-A) wherein each of R₂ and R₃ is methyl, n is 3 or 4 and each R₁ is —(CH₂)₈-A-Y, wherein A is —C(═O)—O— or —O—C(═O)—, and Y is —(CH₂—CH₂—CH(—CH₃)—CH₂)₃—H or —(CH₂)₂—CH═CH—(CH₂)₈—H or —(CH₂)₁₂—H. In some embodiments, a bioreducible or hydrolysable ionizable cationic lipid comprises a compound of Formula (I) or Formula (I-A) wherein each of R₂ and R₃ is methyl, n is 3 or 4 and each R₁ is —(CH₂)₁₀-A-Y, wherein A is —C(═O)—O— or —O—C(═O)—, and Y is —CH₂—CH₂—(CH₂—CH₂—CH(—CH₃)—CH₂)₂—H

In some embodiments, an ionizable cationic lipid comprises the chemical structure of Formula (II):

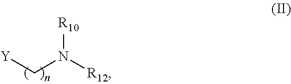

or a pharmaceutically acceptable salt thereof, wherein
   Y is,

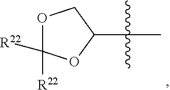

n is an integer 2, 3 or 4;
R²² is a hydrocarbon chain with a single olefin and a total length of C₁₅-C₁₈; and
each of R₁₀ and R₁₂ is independently (C₁-C₄)alkyl optionally substituted with hydroxyl.

In some aspects, R²² in Formula (II) is a polyene hydrorcarbon chain.

In some aspects, R₁₀ and R₁₂ in Formula (II) are each independently selected from methyl, ethyl, propyl, —(CH₂)(CH₂)OH, and —(CH₂)₂(CH₂)OH. In some aspects, R₁₀ and R₁₂ are each independently methyl in Formula (II). In some aspects, R₁₀ and R₁₂ are each independently ethyl in Formula (II). In some aspects, at least one of R₁₀ and R₁₂ is n-propyl optionally substituted with hydroxyl in Formula (II). In some aspects, R₁₀ is methyl and R₁₂ is selected from methyl, ethyl, —(CH₂)(CH₂)OH, and —(CH₂)₂(CH₂)OH in Formula (II). In some aspects, R₁₀ is methyl and R₁₂ is selected from —(CH₂)(CH₂)OH, and —(CH₂)₂(CH₂)OH in Formula (II). In some aspects, R₁₀ is methyl and R₁₂ is selected from —(CH₂)(CH₂)OH, and —(CH₂)₂(CH₂)OH in a compound comprising the chemical structure of Formula (II). In some aspects, R₁₀ and R₁₂ are independently selected from methyl or ethyl, optionally substituted with one or more hydroxyl in Formula (II). In some aspects, one or both of R₁₀ and R₁₂ in Formula (II) are —(CH₂)(CH₂)OH, or —(CH₂)₂(CH₂)OH in Formula (II). In some aspects, R₁₀ is methyl and R₁₂ is methyl or ethyl substituted with hydroxyl in Formula (II). In some aspects, one or both of R₁₀ in Formula (II) is methyl and R₁₂ is —(CH₂)(CH₂)OH in Formula (II). In some aspects, one or both of R₁₀ in Formula (II) is methyl and R₁₂ is —(CH₂)₂(CH₂)OH in Formula (II).

In some embodiments, an LNP comprises an ionizable lipid having a structure of Formula (III), or a pharmaceutically acceptable salt thereof,

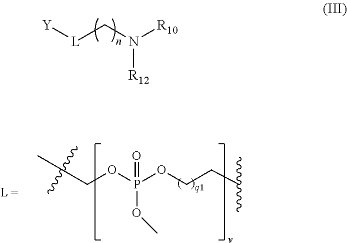

wherein each of R₁₀ and R₁₂ is independently (C₁-C₄)alkyl optionally substituted with hydroxyl; v is 0 or 1; ql is 1 or 2; Y is

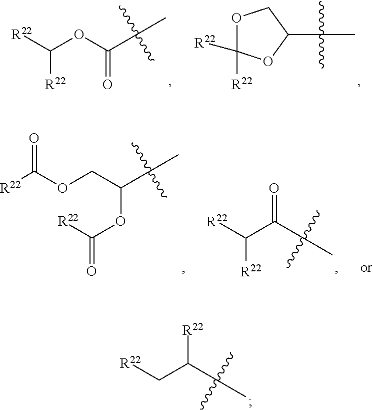

R²² is

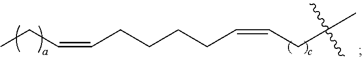

a is 1, 2, 3, 4 or 5; and c is 4, 5, 6, 7 or 8.

In some embodiments, v equals 0 for compounds of Formula (III). In some embodiments, v equals 1 for compounds of Formula (III). In some embodiments, v equals 1 and ql equals 1 for compounds of Formula (III). In some embodiments, v equals 1 and ql equals 2 for compounds of Formula (III).

In some embodiments, the sum of a and c is 6, 7, 8 or 9 in R²² for compounds of Formula (III). In some embodiments, the sum of a and c is 6 in R²² for compounds of Formula (III). In some embodiments, the sum of a and c is 7 in R²² for compounds of Formula (III). In some embodiments, the sum of a and c is 9 in R²² for compounds of Formula (III).

In some embodiments, v equals 0 and the sum of a and c is 6, 7, 8 or 9 in R²² for compounds of Formula (III). In some embodiments, v equals 0 and the sum of a and c is 6 in R²² for compounds of Formula (III). In some embodiments, v equals 0 and the sum of a and c is 7 in R²² for compounds of Formula (III). In some embodiments, v equals 0 and the sum of a and c is 9 in R²² for compounds of Formula (III).

In some embodiments, R₁₀ and R₁₂ are independently selected from methyl, ethyl, —(CH₂)(CH₂)OH, and —(CH₂)₂(CH₂)OH for compounds of Formula (III). In some embodiments, R₁₀ and R₁₂ are each methyl and the sum of a and c is 6, 7, 8 or 9 in R²² for compounds of Formula (III). In some embodiments, R₁₀ and R₁₂ are each methyl, v is 0 and the sum of a and c is 6, 7, 8 or 9 in R²² for compounds of Formula (III).

In some embodiments, v equals 0 and Y is

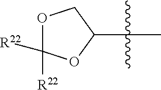

for compounds of Formula (III). In some embodiments, v equals 0 and Y is

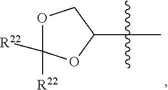

and the sum of a and c is 7 or 9 for compounds of Formula (III). In some embodiments, v equals 0 and Y is

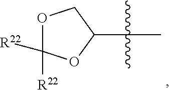

and a is 4 and c is 5 for compounds of Formula (III). In some embodiments, v equals 0 and Y is

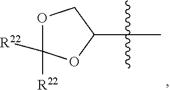

and a is 1 and c is 8 for compounds of Formula (III). In some embodiments, v equals 0 and Y is

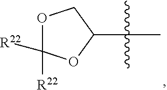

and a is 2 and c is 5 for compounds of Formula (III).

In some embodiments, the LNP composition comprises an ionizable lipid wherein the ionizable lipid comprises:
(a) a dialkyl amino portion of the head group has a chemical structure of Formula (IV-A)

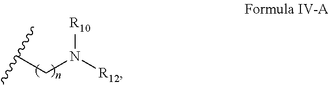

wherein
   n is 2, 3 or 4 in Formula (IV-A); and
   R₁₀ and R₁₂ in Formula (IV-A) are each independently selected from an alkyl group selected from the group consisting of: methyl, ethyl, and propyl, wherein the alkyl in R₁₀ and R₁₂ is optionally substituted with one or more hydroxyl; and
(b) the ionizable lipid further comprises the chemical structure

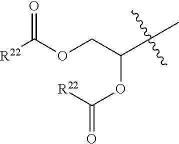

comprising the acyl group of each lipid tail covalently bound to the portion of the head group distal to the dialkyl amino portion of Formula (IV-A), wherein [symbol] indicates attachment to Formula IV-A within the head group, and R²² indicates a portion of each lipid tail covalently bound to the acyl group and having the chemical structure of Formula A:

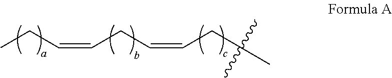

wherein [symbol] in Formula A indicates attachment of Formula A to R²² within each lipid tail; and
   a is 4, 1, 2, or 3;
   b is 4, 2, or 3; and
   c is 4, 3, 5, 6, or 7,
provided that the sum of a, b and c is in Formula A is 12, 10, 11, or 13.

In some embodiments, the ionizable lipid is a compound of Formula (IV-A), wherein R₁₀ and R₁₂ in Formula (IV-A) are each independently methyl, ethyl, —(CH₂)(CH₂)OH, or —(CH₂)₂(CH₂)OH. In some embodiments, the ionizable lipid is a compound of Formula (IV-A), wherein b is 4 and R₁₀ and R₁₂ in Formula (IV-A) are each methyl.

In some embodiments, the compounds have the structure of the compounds listed in the tables below. Table IA show examples of cationic lipids.

TABLE 1A
Exemplary cationic lipids

| | |
|---|---|
| 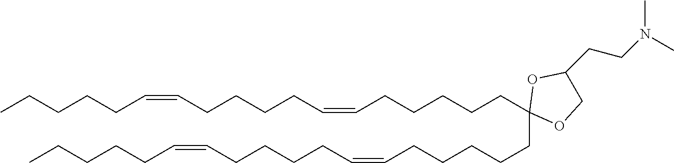 [SMILES: CCCCC/C=C\CCCC/C=C\CCCCCC1(CCCCC/C=C\CCCC/C=C\CCCCC)OCC(CCN(C)C)O1] | 1 |
| 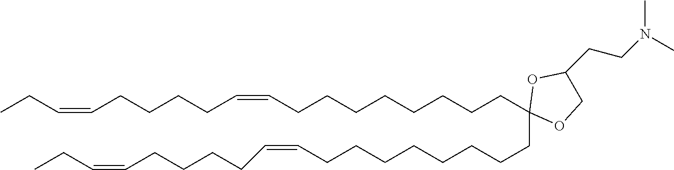 [SMILES: CC/C=C\CCCC/C=C\CCCCCCCCC1(CCCCCCCC/C=C\CCCC/C=C\CC)OCC(CCN(C)C)O1] | 2 |
| 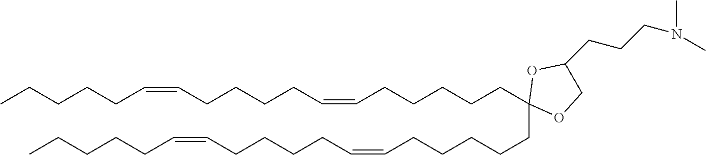 [SMILES: CCCCC/C=C\CCCC/C=C\CCCCCC1(CCCCC/C=C\CCCC/C=C\CCCCC)OCC(CCCN(C)C)O1] | 3 |
| 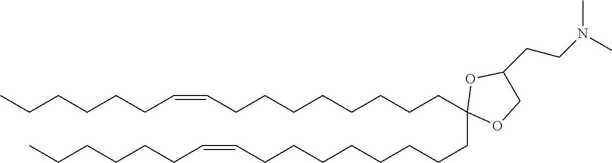 [SMILES: CCCCCC/C=C\CCCCCCCCC1(CCCCCCCC/C=C\CCCCCC)OCC(CCN(C)C)O1] | 4 |
| 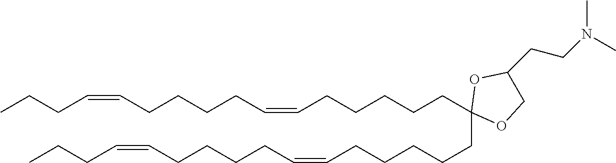 [SMILES: CCC/C=C\CCCC/C=C\CCCCCC1(CCCCC/C=C\CCCC/C=C\CCC)OCC(CCN(C)C)O1] | 5 |
| 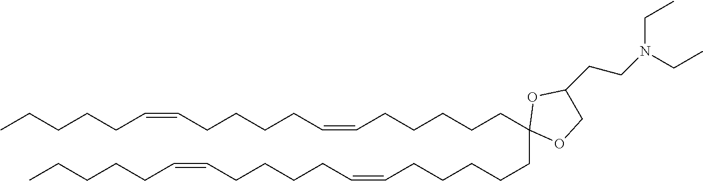 [SMILES: CCCCC/C=C\CCCC/C=C\CCCCCC1(CCCCC/C=C\CCCC/C=C\CCCCC)OCC(CCN(CC)CC)O1] | 6 |
| 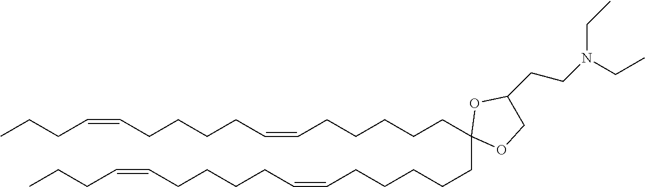 [SMILES: CCC/C=C\CCCC/C=C\CCCCCC1(CCCCC/C=C\CCCC/C=C\CCC)OCC(CCN(CC)CC)O1] | 7 |
| 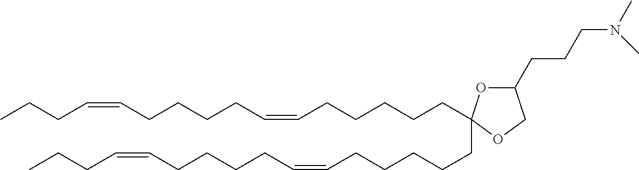 [SMILES: CCC/C=C\CCCC/C=C\CCCCCC1(CCCCC/C=C\CCCC/C=C\CCC)OCC(CCCN(C)C)O1] | 8 |

In some embodiments, a LNP composition comprises an ionizable cationic lipid comprises a pair of identical, lipid hydrocarbon tails having a total of 15, 16, 17 or 18 carbons and comprising a single olefin group, or a pair of olefin groups. In some embodiments, a LNP composition comprises an ionizable cationic lipid selected from the group consisting of:

TABLE 1B

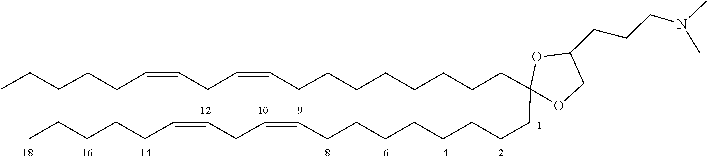 [SMILES: CCCCC/C=C\C/C=C\CCCCCCCCC1(CCCCCCCC/C=C\C/C=C\CCCCC)OCC(CCCN(C)C)O1]

DLIN-KC3-DMA

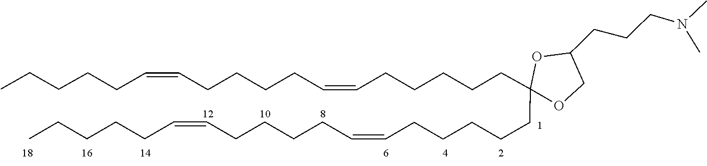 [SMILES: CCCCC/C=C\CCCC/C=C\CCCCCC1(CCCCC/C=C\CCCC/C=C\CCCCC)OCC(CCCN(C)C)O1]

KC3-O1

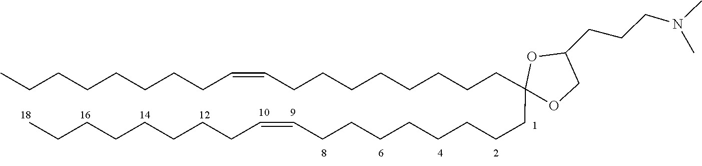 [SMILES: CCCCCCCC/C=C\CCCCCCCCC1(CCCCCCCC/C=C\CCCCCCCC)OCC(CCCN(C)C)O1]

KC3-OA

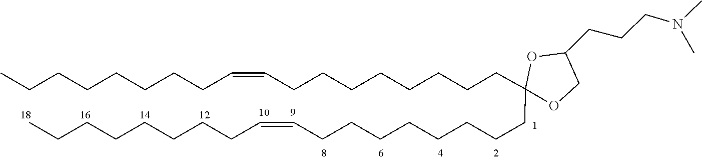 [SMILES: CCCCCCCC/C=C\CCCCCCCCC1(CCCCCCCC/C=C\CCCCCCCC)OCC(CCCN(C)C)O1]

KC3-PA

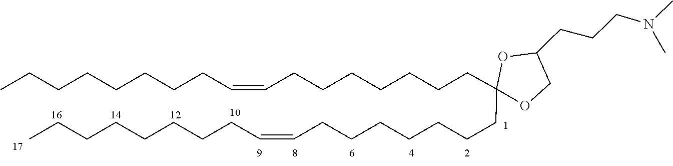 [SMILES: CCCCCCCC/C=C\CCCCCCCC1(CCCCCCC/C=C\CCCCCCCC)OCC(CCCN(C)C)O1]

KC3-C17 (C8:1)

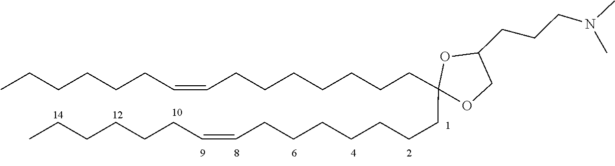 [SMILES: CCCCCC/C=C\CCCCCCCC1(CCCCCCC/C=C\CCCCCC)OCC(CCCN(C)C)O1]

KC3-C15 (C8:1)

In some embodiments, the ionizable cationic lipid is KC3-PA. In some embodiments, the ionizable cationic lipid is KC3-C15 (C8:1). In some embodiments, the ionizable cationic lipid is KC3-C16 (C8:1). In some embodiments, the ionizable cationic lipid is KC3-C17 (C8:1). In some embodiments, the ionizable cationic lipid is KC3-C18 (C8:1). In some embodiments, the ionizable cationic lipid is KC3-15. In some embodiments, the ionizable cationic lipid is KC3-16. In some embodiments, the ionizable cationic lipid is KC3-17. In some embodiments, the ionizable cationic lipid is KC3-18. In some embodiments, the cationic lipid is KC3-OA, KC3-PA, KC3-01, KC3-C₁₇ (8:1), or KC3-C15 (C8:1). In some embodiments, the ionizable cationic lipid is KC3-OA.

In some embodiments, a lipid nanoparticle (LNP) composition comprises an ionizable cationic lipid having the chemical structure of Formula (III) or Formula (IVA), wherein R₁₀ and R₁₂ are the same. In some embodiments, a lipid nanoparticle (LNP) composition comprises an ionizable cationic lipid having the chemical structure of Formula (III) or Formula (IVA), wherein R₁₀ and R₁₂ are each (C₁-C₄)alkyl optionally substituted with hydroxyl. In some embodiments, a lipid nanoparticle (LNP) composition comprises an ionizable cationic lipid having the chemical structure of Formula (III) or Formula (IVA), wherein R₁₀ and R₁₂ are each (C₁-C₄)alkyl. In some embodiments, a lipid nanoparticle (LNP) composition comprises an ionizable cationic lipid having the chemical structure of Formula (III) or Formula (IVA), wherein R₁₀ and R₁₂ are each methyl. In some embodiments, a lipid nanoparticle (LNP) composition comprises an ionizable cationic lipid having the chemical structure of Formula (III) or Formula (IVA), wherein R₁₀ and R₁₂ are each ethyl. In some embodiments, a lipid nanoparticle (LNP) composition comprises an ionizable cationic lipid having the chemical structure of Formula (III) or Formula (IVA), wherein R₁₀ and R₁₂ are each independently selected from methyl or ethyl. In some embodiments, a lipid nanoparticle (LNP) composition comprises an ionizable cationic lipid having the chemical structure of Formula (III) or Formula (IVA), wherein R₁₀ and R₁₂ are each independently selected from methyl, ethyl, —(CH₂)(CH₂)OH, and —(CH₂)₂(CH₂)OH.

In some embodiments, the ionizable cationic lipid is an ionizable cationic lipid from Table 2A:

TABLE 2A
Exemplary ionizable cationic lipids

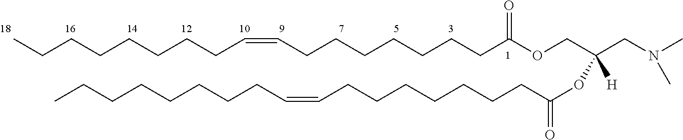 [SMILES: CCCCCCCC/C=C\CCCCCCCC(=O)OC[C@H](CN(C)C)OC(=O)CCCCCCC/C=C\CCCCCCCC]

DODAP

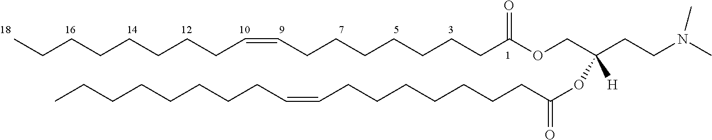 [SMILES: CCCCCCCC/C=C\CCCCCCCC(=O)OC[C@H](CCN(C)C)OC(=O)CCCCCCC/C=C\CCCCCCCC]

AKG-OA-DM2

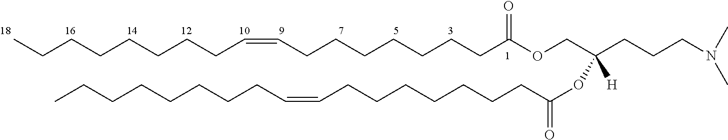 [SMILES: CCCCCCCC/C=C\CCCCCCCC(=O)OC[C@H](CCCN(C)C)OC(=O)CCCCCCC/C=C\CCCCCCCC]

AKG-OA-DM3

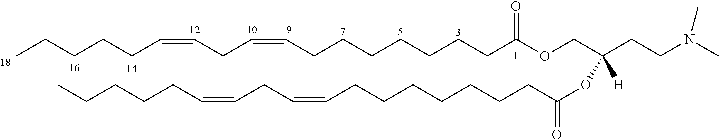 [SMILES: CCCCC/C=C\C/C=C\CCCCCCCC(=O)OC[C@H](CCN(C)C)OC(=O)CCCCCCC/C=C\C/C=C\CCCCC]

O-11769

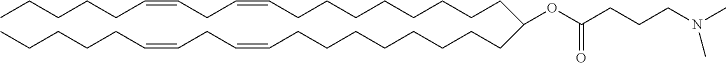 [SMILES: CCCCC/C=C\C/C=C\CCCCCCCCC(CCCCCCCC/C=C\C/C=C\CCCCC)OC(=O)CCCN(C)C]

Dlin-MC3-DMA

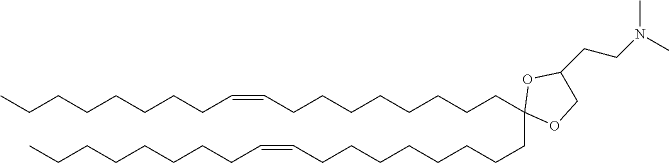 [SMILES: CCCCCCCC/C=C\CCCCCCCCC1(CCCCCCCC/C=C\CCCCCCCC)OCC(CCN(C)C)O1]

AKG-KC2-OA

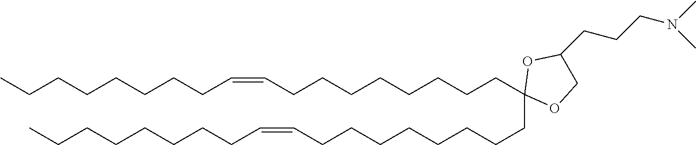 [SMILES: CCCCCCCC/C=C\CCCCCCCCC1(CCCCCCCC/C=C\CCCCCCCC)OCC(CCCN(C)C)O1]

AKG-KC3-OA

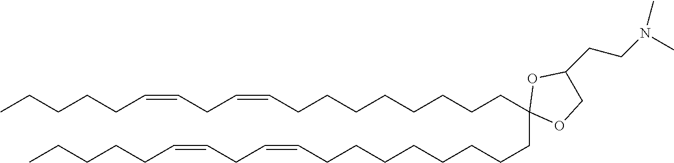 [SMILES: CCCCC/C=C\C/C=C\CCCCCCCCC1(CCCCCCCC/C=C\C/C=C\CCCCC)OCC(CCN(C)C)O1]

Dlin-KC2-DMA

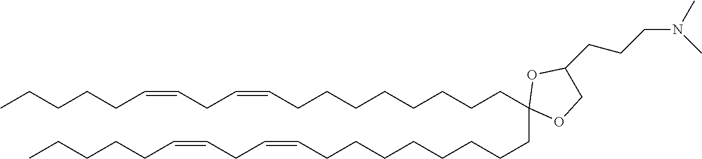 [SMILES: CCCCC/C=C\C/C=C\CCCCCCCCC1(CCCCCCCC/C=C\C/C=C\CCCCC)OCC(CCCN(C)C)O1]

Dlin-KC3-DMA

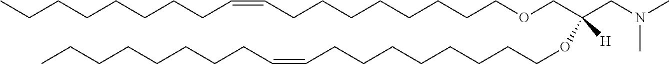 [SMILES: CCCCCCCC/C=C\CCCCCCCCOC[C@H](CN(C)C)OCCCCCCCC/C=C\CCCCCCCC]

DODMA

In some embodiments, the ionizable cationic lipid can be a branched ionizable lipid selected from ALC-0315 and SM-102:

TABLE 2B

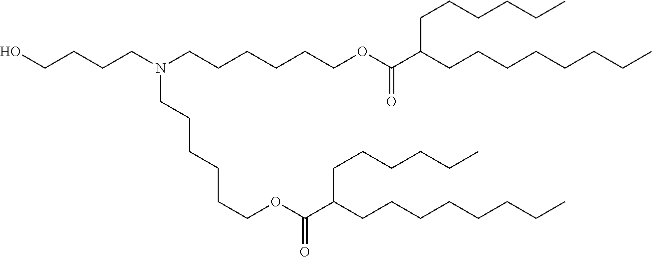 [SMILES: CCCCCCCCC(CCCCCC)C(=O)OCCCCCCN(CCCCO)CCCCCCOC(=O)C(CCCCCC)CCCCCCCC]

ALC-0315

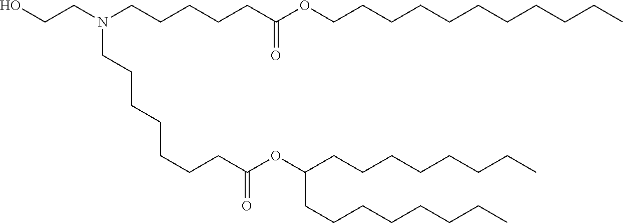 [SMILES: CCCCCCCCCCCOC(=O)CCCCCN(CCO)CCCCCCCC(=O)OC(CCCCCCCC)CCCCCCCC]

SM-102

In some embodiments the percentage of oxidative degradation products for the ionizable cationic lipid is less than 50% of that for a DLin-KC2-DMA or DLin-MC3-DMA control formulation.

An LNP composition may comprise an ionizable lipid at a concentration greater than about 0.1 mol %, e.g., of the total lipid content of the LNP. In an embodiment, the LNP comprises an ionizable lipid at a concentration of greater than about 1 mol %, about 2 mol %, about 4 mol %, about 8 mol %, about 20 mol %, about 40 mol %, about 50 mol %, about 60 mol %, about 80 mol %, e.g., of the total lipid content of the LNP. In an embodiment, the LNP comprises an ionizable lipid at a concentration of greater than about 20 mol %, about 40 mol %, or about 50 mol %. In an embodiment, the LNP comprises an ionizable lipid at a concentration between about 1 mol % to about 95 mol %, e.g., of the total lipid content of the LNP. In an embodiment, the LNP comprises an ionizable lipid at a concentration between about 2 mol % to about 90 mol %, about 4 mol % to about 80 mol %, about mol % to about 70 mol %, about 20 mol % to about 60 mol %, about 40 mol % to about 55 mol %, e.g., of the total lipid content of the LNP. In an embodiment, the LNP comprises an ionizable lipid at a concentration between about 20 mol % to about 60 mol %. In an embodiment, the LNP comprises an ionizable lipid at a concentration between about 40 mol % to about 55 mol %.

### (C) Phospholipids

In some embodiments, the LNP composition comprises one or more phospholipids. A phospholipid is a lipid that comprises a phosphate group and at least one alkyl, alkenyl, or heteroalkyl chain. A phospholipid may be naturally occurring or non-naturally occurring (e.g., a synthetic phospholipid). A phospholipid may comprise an amine, amide, ester, carboxyl, choline, hydroxyl, acetal, ether, carbohydrate, sterol, or a glycerol. In some embodiments, a phospholipid may comprise a phosphocholine, phosphosphingolipid, or a plasmalogen. Exemplary phospholipids include 1,2-dioleoyl-sn-glycero-3-phosphocholine (DOPC), 1,2-dipalmitoyl-sn-glycero-3-phosphocholine (DPPC), 1,2-dioleoyl-sn-glycero-3-phosphoethanolamine (DOPE), 1,2-distearoyl-sn-glycero-3-phosphocholine (DSPC), hydrogenated soy phosphatidylcholine (HSPC), 1,2-dilauroyl-sn-glycero-3-phosphocholine (DLPC), 1,2-dimyristoyl-sn-glycero-3-phosphocholine (DMPC), 1,2-distearoyl-sn-glycero-3-phosphoethanolamine (DSPE), 1-myristoyl-2-oleoyl-sn-glycero-3-phosphocholine (MOPC), 1,2-diarachidonoyl-sn-glycero-3-phosphocholine (DAPC), 1-palmitoyl-2-linoleoyl-sn-glycero-3-phosphatidylcholine (PLPC), 1-palmitoyl-2-oleoyl-glycero-3-phosphocholine (POPC), 1-stearoyl-2-myristoyl-sn-glycero-3-phosphocholine (SMPC), 1-palmitoyl-2-myristoyl-sn-glycero-3-phosphocholine (PMPC), bis(monoacylglycerol)phosphate (BMP), L-a-phosphatidylcholine, 1,2-Diheptadecanoyl-sn-glycero-3-phosphorylcholine (DHDPC), and 1-stearoyl-2-arachidonoyl-sn-glycero-3-phosphocholine (SAPC). Additional phospholipids that may be included in an LNP described herein are disclosed in Li, J. et al. (Asian J. Pharm. Sci. 10:81-98 (2015)), which is incorporated herein by reference in its entirety.

In some aspects, the one or more phospholipids comprises distearoylphosphatidylcholine (DSPC), hydrogenated soy phosphatidylcholine (HSPC), dipalmitoylphosphatidylcholine (DPPC) or a combination thereof. In some embodiments, the phospholipid is 1,2-distearoyl-sn-glycero-3-phosphocholine (DSPC). In some embodiments, the phospholipid is 1,2-dioleoyl-sn-glycero-3-phosphocholine(DOPC). In some embodiments, the phospholipid is 1,2-dipalmitoyl-sn-glycero-3-phosphocholine(DPPC). In some embodiments, the phospholipid is 1,2-dioleoyl-sn-glycero-3-phosphoethanolamine (DOPE). In some embodiments, the phospholipids consist of one or more phospholipids selected from the group consisting of: DSPC, DOPC, DPPC, HSPC, and SM.

A LNP composition may comprise one or more phospholipid(s) at a concentration greater than about 0.1 mol %, e.g., of the total lipid content of the LNP. In an embodiment, the LNP comprises a total phospholipid at a concentration of greater than about 0.5 mol %, about 1 mol %, about 1.5 mol %, about 2 mol %, about 3 mol %, about 4 mol %, about 5 mol %, about 6 mol %, about 8 mol %, about 10 mol %, about 12 mol %, about 15 mol %, about 20 mol %, about 50 mol %, e.g., of the total lipid content of the LNP. In an embodiment, the LNP comprises a total phospholipid at a concentration of greater than about 1 mol %, about 5 mol %, or about 10 mol %. In an embodiment, the LNP comprises a phospholipid at a concentration between about 0.1 mol % to about 50 mol %, e.g., of the total lipid content of the LNP. In an embodiment, the LNP comprises a phospholipid at a concentration between about 0.5 mol % to about 40 mol %, about 1 mol % to about 30 mol %, about 5 mol % to about 25 mol %, about 10 mol % to about 20 mol %, about 10 mol % to about 15 mol %, or about 15 mol % to about 20 mol %, e.g., of the total lipid content of the LNP. In an embodiment, the LNP comprises a phospholipid at a concentration between about 5 mol % to about 25 mol %. In an embodiment, the LNP comprises a phospholipid at a concentration between about 10 mol % to mol %.

### (D) Anionic Targeting Phospholipids

In some embodiments, LNP compositions comprise a targeting ligand directed against cell surface receptors to target lipid nanoparticles in a highly specific manner, including to dendritic cells. Some aspects of the disclosure relate to LNP comprising a ligand (also referred herein as targeting ligand) having a binding specificity for a cell surface antigen, wherein the binding of the ligand to the antigen induces the internalization of the ligand. Some embodiments relate to compositions comprising LNP comprising a ligand as described herein. LNP targeting can also accomplished by adding lipids to the formulation. For example, phosphatidylserine is known to redistribute to the external surface of the plasma membrane during apoptosis and is a molecular cue for phagocytotic cell attraction (Fadok et al. Curr Biol. 2003 Aug. 19; 13(16):R655-7).

In some embodiments, a LNP composition can further comprise an anionic phospholipid as a targeting moiety. Phosphatidylserine (PS) and phosphatidylglycerol (PG) are recognized by dendritic cells and can induce uptake and activation of dendritic cells LNP targeting can also accomplished by adding certain anionic phospholipids to the formulation. For example, phosphatidylserine is known to redistribute to the external surface of the plasma membrane during apoptosis and is a molecular cue for phagocytotic cell attraction (Fadok et al. Curr Biol. 2003 Aug. 19; 13(16):R655-7). Phosphatidylserine (PS) and phosphatidylglycerol (PG) are recognized by dendritic cells and can induce uptake and activation of dendritic cells (Caronni et al., Nat Comm. 2021 April 14; 12: 2237-2253; Ischihashi et al., PLOS One 2013). Although anionic phospholipids have been used previously in the context of liposomes, their inclusion in lipidic nanoparticles that include condensed nucleic acids is unexpected since anionic headgroups may compete for binding sites of the ionizable cationic lipids with the phosphate backbone of mRNA, may inhibit intracellular escape by altering the surface charge, or may result in aggregation of LNPs during formation or storage.

Some embodiments relate to the use of a (L-Serine) PS lipid and/or a phosphatidylglycerol (PG) anionic phospholipid in combination with an ionizable cationic lipid for targeting of the LNP to dendritic or other cells. In some embodiments, a LNP composition comprises one or more phospholipids in a total amount of phospholipids of 5-25 mol % of the total lipid content of the LNP composition, and comprising a phosphatidylglycerol (PG) in a total amount of 1.0-10 mol % of the total lipid content of the LNP composition; and (e) a conjugated lipid in a total amount of 0.5-2.5 mol % of the total lipid content of the LNP composition.

In some embodiments, the LNP composition comprises a phosphatidyl-L-serine compound as a targeting ligand, such as dipalmitoylphosphatidyl-L-serine (DPPS), or distearoylphosphatidyl-L-serine (DSPS). In some embodiments, the LNP composition comprises a phosphatidyl-L-serine compound as a targeting ligand and an anionic phospholipid. In some embodiments, the PS lipid is (L-Serine) DSPS, (L-Serine) DPPS, or a mixture thereof, and the LNP composition further comprises cholesterol and a second phospholipid selected from the group consisting of: DSPC, DOPC, DPPC, HSPC, and SM. In some embodiments, LNP compositions comprise both a phosphatidyl-L-serine compound as a targeting ligand, and distearoylphosphatidylcholine (DSPC) as the second phospholipid. In some embodiments, LNP compositions comprise both a phosphatidyl-L-serine compound as a targeting ligand, and distearoylphosphatidylcholine (DSPC) as the second phospholipid without dipalmitoylphosphatidylcholine (DPPC). In some embodiments, the PS lipid is one or more L-serine lipids selected from the group consisting of DPPS and DSPS.

In some embodiments, the one or more phospholipids comprise at least two (L-Serine) PS lipids having mismatched acyl chain lengths. In some embodiments, the one or more phospholipids comprise at least two (L-Serine) PS lipids having identical acyl chain lengths.

In some embodiments, the composition comprises the PS lipid in a total amount selected from 1.25 mol %, 2.5 mol %, 5 mol %, 7.5 mol %, and 10 mol % of the total lipid content of the LNP composition.

In some embodiments, the LNP composition comprises a phosphatidylglycerol-containing compound as a targeting ligand such as distearoylphosphatidylglycerol (DSPG) or dipalmitoyphosphatidylglycerol (DPPG), for enhancing expression in human dendritic cells. In some embodiments, the anionic phospholipid used is phosphatidylglycerol. In another embodiment, the acyl chains for the phosphatidylglycerol are fully saturated, such as the case for dimyristoylphosphatidylglycerol (DMPG), dipalmitoylphosphatidylglycerol (DPPG), or distearoylphosphatidylglycerol (DSPG). In a preferred embodiment, the PG used is either DPPG or DSPG. The phosphatidylglycerol may also contain an asymmetric acyl chain composition, for example where one acyl chain is stearic acid and another is palmitic acid.

In some aspects, the one or more anionic phospholipids is a phosphatidylserine (PS) and/or phosphatidylglycerol (PG). In some aspects, the one or more anionic phospholipids is selected from the group consisting of: dipalmitoylphosphatidyl-L-serine (DPPS), or distearoylphosphatidyl-L-serine (DSPS), distearoylphosphatidylglycerol (DSPG), and dipalmitoyphosphatidylglycerol (DPPG).

In some embodiments, the anionic targeting ligands are selected from the group, phosphatidylserine (PS), phoshatidylglycerol (PG), N-glutaryl-phosphatidylethanolamine (N-glu-PE), or N-succinyl-phosphatidylethanolamine (N-Suc-PE). In some embodiments, the anionic phospholipid used is phosphatidylserine. In another embodiment, the phosphatidylserine contains the L-isomer of serine. In another embodiment, the acyl chains for the phosphatidylserine are fully saturated, such as the case for dimyristoylphosphatidyl-L-serine (DMPS), dipalmitoylphosphatidyl-L-serine (DPPS), or distearoylphosphatidyl-L-serine (DSPS). In a preferred embodiment, the PS used is the L-isomer of dioleoylphosphatidylserine (DOPS). The phosphatidylserine may also contain an asymmetric acyl chain composition, for example where one acyl chain is stearic acid and another is palmitic acid.

In some embodiments, the one or more phospholipids comprise a phosphatidylserine (PS) lipid. In some embodiments, the PS lipid in the LNP composition is DPPS. In some embodiments, the PS lipid in the LNP composition is present in a total of 5 mol %. In some embodiments, the LNP composition comprises a DSPC phospholipid. In some embodiments, the LNP composition comprises 7.5-20 mol % of a DSPC phospholipid

TABLE 3A
Anionic Phospholipid Targeting Moieties

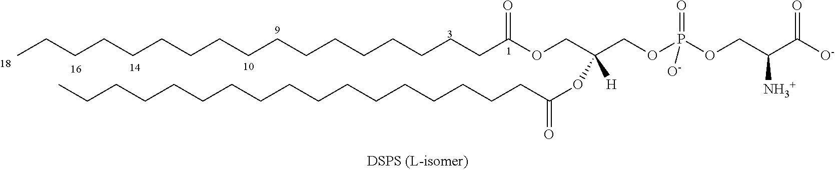 [SMILES: CCCCCCCCCCCCCCCCCC(=O)OC[C@H](COP(=O)([O-])OC[C@H]([NH3+])C(=O)[O-])OC(=O)CCCCCCCCCCCCCCCCC]

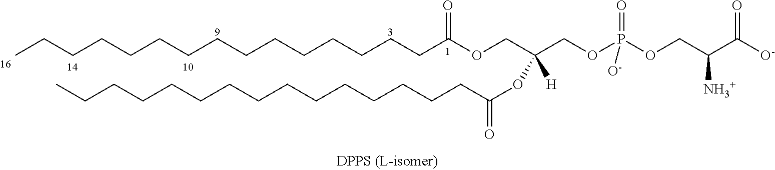 [SMILES: CCCCCCCCCCCCCCCC(=O)OC[C@H](COP(=O)([O-])OC[C@H]([NH3+])C(=O)[O-])OC(=O)CCCCCCCCCCCCCCC]

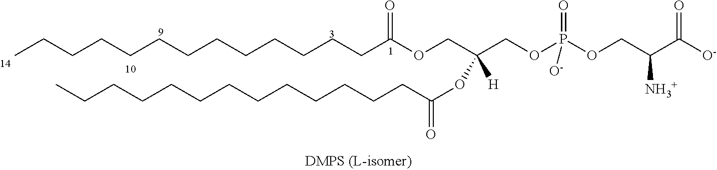 [SMILES: CCCCCCCCCCCCCC(=O)OC[C@H](COP(=O)([O-])OC[C@H]([NH3+])C(=O)[O-])OC(=O)CCCCCCCCCCCCC]

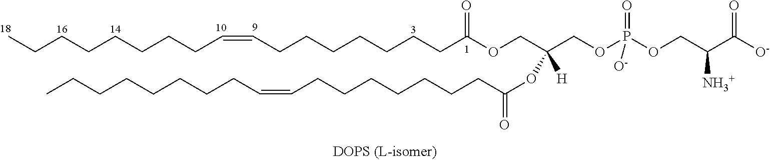 [SMILES: CCCCCCCC/C=C\CCCCCCCC(=O)OC[C@H](COP(=O)([O-])OC[C@H]([NH3+])C(=O)[O-])OC(=O)CCCCCCC/C=C\CCCCCCCC]

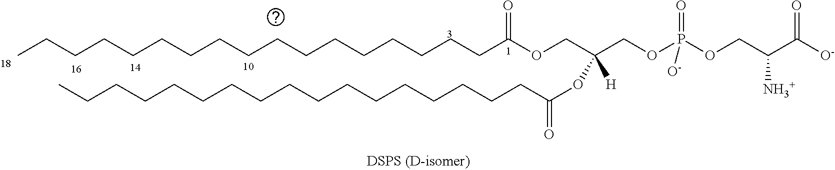 [SMILES: CCCCCCCCCCCCCCCCCC(=O)OC[C@H](COP(=O)([O-])OC[C@@H]([NH3+])C(=O)[O-])OC(=O)CCCCCCCCCCCCCCCCC]

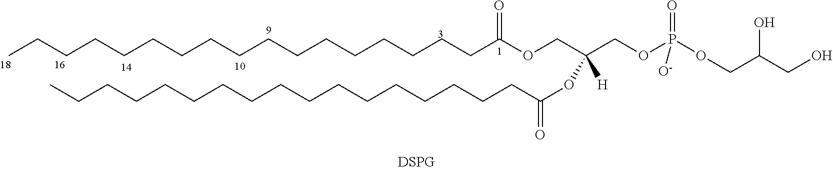 [SMILES: CCCCCCCCCCCCCCCCCC(=O)OC[C@H](COP(=O)([O-])OCC(O)CO)OC(=O)CCCCCCCCCCCCCCCCC]

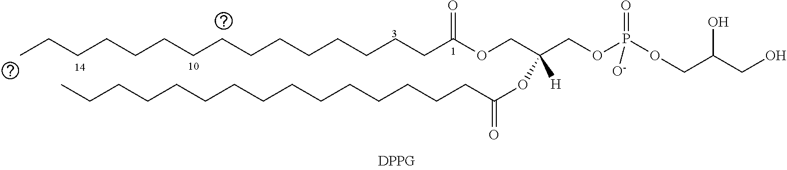 [SMILES: CCCCCCCCCCCCCCCC(=O)OC[C@H](COP(=O)([O-])OCC(O)CO)OC(=O)CCCCCCCCCCCCCCC]

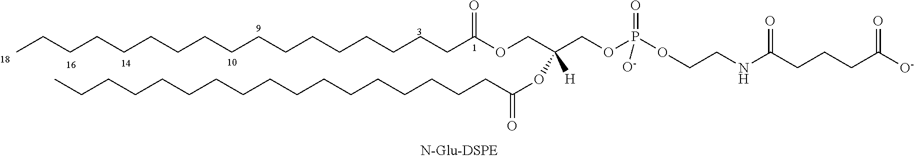 [SMILES: CCCCCCCCCCCCCCCCCC(=O)OC[C@H](COP(=O)([O-])OCCNC(=O)CCCC(=O)[O-])OC(=O)CCCCCCCCCCCCCCCCC]

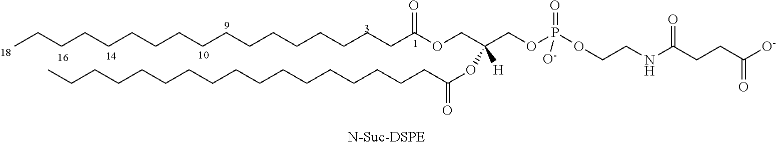 [SMILES: CCCCCCCCCCCCCCCCCC(=O)OC[C@H](COP(=O)([O-])OCCNC(=O)CCC(=O)[O-])OC(=O)CCCCCCCCCCCCCCCCC]

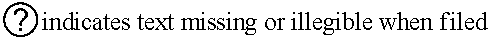

TABLE 3B
Nonphosphatidylserine anionic phospholipds

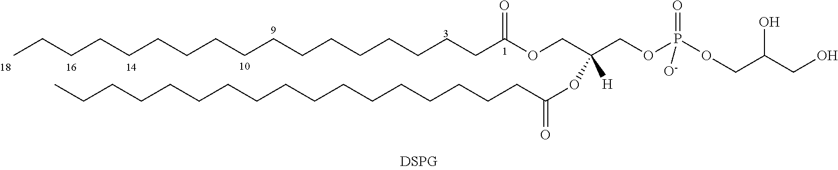 [SMILES: CCCCCCCCCCCCCCCCCC(=O)OC[C@H](COP(=O)([O-])OCC(O)CO)OC(=O)CCCCCCCCCCCCCCCCC]

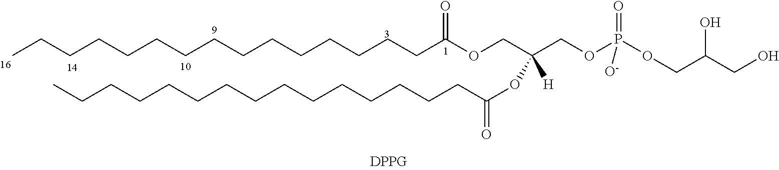 [SMILES: CCCCCCCCCCCCCCCC(=O)OC[C@H](COP(=O)([O-])OCC(O)CO)OC(=O)CCCCCCCCCCCCCCC]

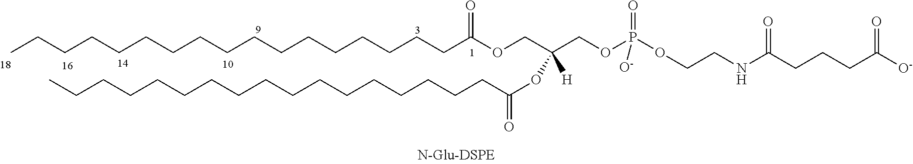 [SMILES: CCCCCCCCCCCCCCCCCC(=O)OC[C@H](COP(=O)([O-])OCCNC(=O)CCCC(=O)[O-])OC(=O)CCCCCCCCCCCCCCCCC]

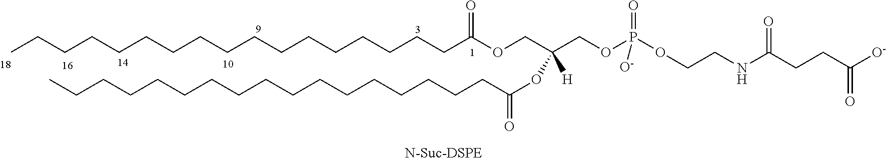 [SMILES: CCCCCCCCCCCCCCCCCC(=O)OC[C@H](COP(=O)([O-])OCCNC(=O)CCC(=O)[O-])OC(=O)CCCCCCCCCCCCCCCCC]

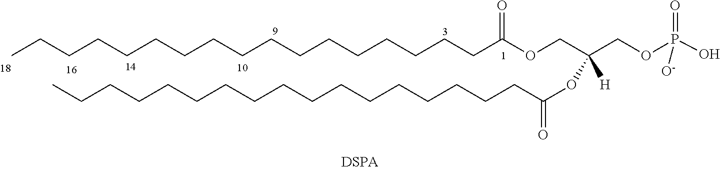 [SMILES: CCCCCCCCCCCCCCCCCC(=O)OC[C@H](COP(=O)([O-])O)OC(=O)CCCCCCCCCCCCCCCCC]

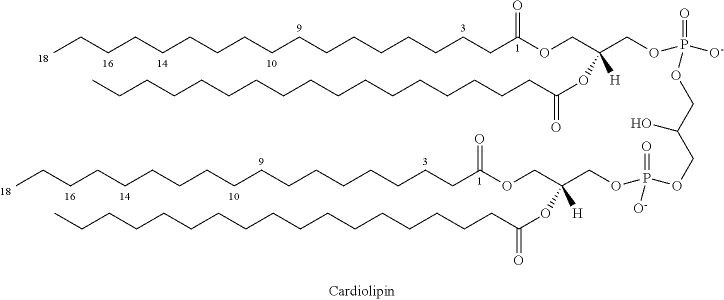 [SMILES: CCCCCCCCCCCCCCCCCC(=O)OC[C@H](COP(=O)([O-])OCC(O)COP(=O)([O-])OC[C@@H](COC(=O)CCCCCCCCCCCCCCCCC)OC(=O)CCCCCCCCCCCCCCCCC)OC(=O)CCCCCCCCCCCCCCCCC]

In some embodiments, the composition comprises an anionic targeting phospholipid other than phosphatidyl-L-serine. Other or additional anionic phospholipids, separate from phosphatidyl-L-serine, can also be considered as targeting lipids for LNPs. These include phosphatidylglycerol (PG), phosphatidic acid (PA), N-glutaryl-phosphatidylethanolamine (N-Glu-PE), N-succinyl-phosphatidylethanolamine (N-Suc-PE), and cardiolipin. In some embodiments, a LNP comprises anionic phospholipids, separate from phosphatidyl-L-serine, useful as targeting lipids for LNPs. In some embodiments, a LNP comprises anionic phospholipids selected from the group consisting of: phosphatidylglycerol (PG), phosphatidic acid (PA), N-glutaryl-phosphatidylethanolamine (N-Glu-PE), N-succinyl-phosphatidylethanolamine (N-Suc-PE), and cardiolipin. Distearoylphosphatidylglycerol (DSPG), dipalmitoyphosphatidylglycerol (DPPG), N-succinyl-distearoylphosphatidylethanolamine (N-Suc-DSPE), N-glutaryl-distearoylphosphatidylethanolamine (N-glu-DSPE), distearoylphosphatidic acid (DSPA), and cardiolipin are also provided as anionic phospholipids. In some embodiments, the composition comprises an anionic phospholipid selected from the group consisting of: DOPG, DSPG and DPPG. In some embodiments, the composition comprises an anionic phospholipid selected from the group consisting of: N-Glu-DSPE and N-Suc-DSPE. In some embodiments, the composition comprises a DSPA anionic phospholipid. In some embodiments, the composition comprises a Cardiolipin anionic phospholipid.

In some embodiments, LNP compositions further comprising ligands, such as antibody conjugates, directed against cell surface receptors to target lipid nanoparticles in a highly specific manner to dendritic cells are provided. In some embodiments, the composition further comprises a targeting ligand, wherein the targeting ligand is oriented to the outside of the nanoparticle. In some embodiments, the targeting ligand is an antibody.

### (E) Conjugated Lipid

In some embodiments, the LNP comprises an alkylene glycol-containing lipid. An alkylene glycol-containing lipid is a lipid that comprises at least one alkylene glycol moiety, for example, a methylene glycol or an ethylene glycol moiety. In some embodiments, the alkylene glycol-containing lipid comprises a polyethylene glycol (PEG). An alkylene glycol-containing lipid may be a PEG-containing lipid. Polymer-conjugated lipids may include poly(ethylene glycol)-conjugated (pegylated)phospholipids (PEG-lipids) such as PEG(Mol. weight 2,000) methoxy-poly(ethylene glycol)-1,2-distearoyl-sn-glycerol (PEG-DSG), PEG(Mol. weight 2,000) methoxy-poly(ethylene glycol)-1,2-palmitoyl-sn-glycerol (PEG-DPG), PEG(Mol. weight 2,000) 1,2-distearoyl-sn-glycero-3-phosphoethanolamine-N-[methoxy(polyethylene glycol)-2000](PEG-DSPE) or N-palmitoyl-sphingosine-1-{succinyl[methoxy(polyethylene glycol)2000]}(PEG-ceramide). The molecular weight of the PEG portion in the PEG-lipid component can also vary from 500-10,000 g/mol, from 1,500-6000 g/mol, but is preferably about 2,000 MW. Other polymers used for conjugation to lipid anchors may include poly(2-methyl-2-oxazoline) (PMOZ), poly(2-ethyl-2-oxazoline) (PEOZ), poly-N-vinylpyrrolidone (PVP), polyglycerol, poly(hydroxyethyl L-asparagine) (PHEA), and poly(hydroxyethyl L-glutamine) (PHEG).

A PEG-containing lipid may further comprise an amine, amide, ester, carboxyl, phosphate, choline, hydroxyl, acetal, ether, heterocycle, or carbohydrate. PEG-containing lipids may comprise at least one alkyl or alkenyl group, e.g., greater than six carbon atoms in length (e.g., greater than about 8 carbons, 10 carbons, 12 carbons, 14 carbons, 16 carbons, 18 carbons, 20 carbons or more in length), e.g., in addition to a PEG moiety. In an embodiment, a PEG-containing lipid comprises a PEG moiety comprising at least 20 PEG monomers, e.g., at least 30 PEG monomers, 40 PEG monomers, 45 PEG monomers, 50 PEG monomers, 100 PEG monomers, 200 PEG monomers, 300 PEG monomers, 500 PEG monomers, 1000 PEG monomers, or 2000 PEG monomers. Exemplary PEG-containing lipids include PEG-DMG (e.g., DMG-PEG2k), PEG-c-DMG, PEG-DSG, PEG-DPG, PEG-DSPE, PEG-DMPE, PEG-DPPE, PEG-DOPE, and PEG-DLPE. In some embodiments, the PEG-lipids include PEG-DMG (e.g., DMG-PEG2k), PEG-c-DMG, PEG-DSG, and PEG-DPG. Additional PEG-lipids that may be included in an LNP described herein are disclosed in Fahy, E. et al. (J. Lipid. Res. 46:839-862 (2005) which is incorporated herein by reference in its entirety.

In some embodiments, the PEG-lipid is PEG-DMG (e.g., DMG-PEG2k). In some embodiments, the PEG-lipid is α-(3′-{[1,2-di(myristyloxy)propanoxy]carbonylamino}propyl)-co-methoxy, polyoxyethylene (PEG-c-DMG). In some embodiments, the PEG-lipid is PEG-DSG. In some embodiments, the PEG-lipid is PEG-DPG. In some embodiments, the polymer-conjugated lipid is PEG(2000)-dimyristoylglycerol (PEG-DMG) or PEG(Mol. weight 2,000)-dimyristoylphosphatidylethanolamine (PEG-DMPE). In some aspects, the conjugated lipid is PEG(2000)-dimyristoylglycerol (PEG-DMG). In some aspects, the sterol is cholesterol.

An LNP may comprise an alkylene glycol-containing lipid at a concentration greater than about 0.1 mol %, e.g., of the total lipid content of the LNP. In an embodiment, the LNP comprises an alkylene glycol-containing lipid at a concentration of greater than about 0.5 mol %, about 1 mol %, about 1.5 mol %, about 2 mol %, about 3 mol %, about 4 mol %, about 5 mol %, about 6 mol %, about 8 mol %, about 10 mol %, about 12 mol %, about 15 mol %, about 20 mol %, about 50 mol %, e.g., of the total lipid content of the LNP. In an embodiment, the LNP comprises an alkylene glycol-containing lipid at a concentration of greater than about 1 mol %, about 4 mol %, or about 6 mol %. In an embodiment, the LNP comprises an alkylene glycol-containing lipid at a concentration between about 0.1 mol % to about 50 mol %, e.g., of the total lipid content of the LNP. In an embodiment, the LNP comprises an alkylene glycol-containing lipid at a concentration between about 0.5 mol % to about 40 mol %, about 1 mol % to about 35 mol %, about 1.5 mol % to about mol %, about 2 mol % to about 25 mol %, about 2.5 mol % to about 20%, about 3 mol % to about mol %, about 3.5 mol % to about 10 mol %, or about 4 mol % to 9 mol %, e.g., of the total lipid content of the LNP. In an embodiment, the LNP comprises an alkylene glycol-containing lipid at a concentration between about 3.5 mol % to about 10 mol %. In an embodiment, the LNP comprises an alkylene glycol-containing lipid at a concentration between about 4 mol % to 9 mol %.

In some embodiments, the LNP comprises the conjugated lipid in a total amount of less than 2 mol % of the total lipid content of the LNP composition. In some embodiments, the LNP comprises the conjugated lipid in a total amount of 0.5-2.0 mol % of the total lipid content of the LNP composition. In some embodiments, the conjugated lipid in a total amount of less than 2 mol % of the total lipid content of the LNP composition. In some embodiments, the LNP composition further 0.5-2.0 mol % PEG-DMG or PEG-DSG, based on the total lipid content in the LNP composition.

In some embodiments, the PEG-containing conjugated lipid in the LNP composition is PEG-DMG. In some embodiments, the LNP composition comprises 1.5-4.0 mol % PEG-containing conjugated lipid in the LNP composition is PEG-DMG. In some embodiments, the PEG-containing conjugated lipid in the LNP composition is PEG-DLG. In some embodiments, the LNP composition comprises 1.0-4.0 mol % PEG-containing conjugated lipid in the LNP composition is PEG-DLG.

### (F) Sterol

In an embodiment, the LNP comprises a sterol or ionizable sterol molecule. A sterol is a lipid that comprises a polycyclic structure and an optionally a hydroxyl or ether substituent, and may be naturally occurring or non-naturally occurring (e.g., a synthetic sterol). Sterols may comprise no double bonds, a single double bond, or multiple double bonds. Sterols may further comprise an alkyl, alkenyl, halo, ester, ketone, hydroxyl, amine, polyether, carbohydrate, or cyclic moiety. An exemplary listing of sterols includes cholesterol, dehydroergosterol, ergosterol, campesterol, β-sitosterol, stigmasterol, lanosterol, dihydrolanosterol, desmosterol, brassicasterol, lathosterol, zymosterol, 7-dehydrodesmosterol, avenasterol, campestanol, lupeol, and cycloartenol. In some embodiments, the sterol comprises cholesterol, dehydroergosterol, ergosterol, campesterol, β-sitosterol, or stigmasterol. Additional sterols that may be included in an LNP described herein are disclosed in Fahy, E. et al. (J. Lipid. Res. 46:839-862 (2005)).

In some embodiments, an LNP comprises a sterol. In some embodiments, the sterol is cholesterol. In some embodiments, the sterol is dehydroergosterol. In some embodiments, the sterol is ergosterol. In some embodiments, the sterol is campesterol. In some embodiments, the sterol is β-sitosterol. In some embodiments, the sterol is stigmasterol. In some embodiments, the sterol is a corticosteroid. (e.g., corticosterone, hydrocortisone, cortisone, or aldosterone).

An LNP may comprise a sterol at a concentration greater than about 0.1 mol %, e.g., of the total lipid content of the LNP. In an embodiment, the LNP comprises a sterol at a concentration greater than about 0.5 mol %, about 1 mol %, about 5 mol %, about 10 mol %, about 15 mol %, about mol %, about 25 mol %, about 35 mol %, about 40 mol %, about 45 mol %, about 50 mol %, about 55 mol %, about 60 mol %, about 65 mol %, or about 70 mol %, e.g., of the total lipid content of the LNP. In an embodiment, the LNP comprises a sterol at a concentration greater than about 10 mol %, about 15 mol %, about 20 mol %, or about 25 mol %. In an embodiment, the LNP comprises a sterol at a concentration between about 1 mol % to about 95 mol %, e.g., of the total lipid content of the LNP. In an embodiment, the LNP comprises a sterol at a concentration between about 5 mol % to about 90 mol %, about 10 mol % to about 85 mol %, about 20 mol % to about 80 mol %, about 20 mol % to about 60 mol %, about 20 mol % to about 50 mol %, or about 20 mol % to 40 mol %, e.g., of the total lipid content of the LNP. In an embodiment, the LNP comprises a sterol at a concentration between about 20 mol % to about 50 mol %. In an embodiment, the LNP comprises a sterol at a concentration between about 30 mol % to about 60 mol %.

In some embodiments, the sterol in the LNP composition is cholesterol. In some embodiments, the LNP composition comprises 25-40 mol % cholesterol. In some embodiments, the sterol in the LNP composition is beta sitosterol. In some embodiments, the LNP composition comprises 33-35.5 mol % beta sitosterol.

In some embodiments, the composition comprises the cholesterol in a total amount of 23.5 mol % of the total lipid content of the LNP composition. In some embodiments, the composition comprises the cholesterol in a total amount of 33.5 mol % of the total lipid content of the LNP composition. In some embodiments, the composition comprises the cholesterol in a total amount of 38.5 mol % of the total lipid content of the LNP composition. In some embodiments, the composition comprises the cholesterol in a total amount of 40.5 mol % of the total lipid content of the LNP composition. In some embodiments, the composition comprises the cholesterol in a total amount of 42.7 mol % of the total lipid content of the LNP composition. In some embodiments, the composition comprises the cholesterol in a total amount of 43.5 mol % of the total lipid content of the LNP composition. In some embodiments, the composition comprises the cholesterol in a total amount of 33.5-43.5 mol % of the total lipid content of the LNP composition. In some embodiments, the composition comprises the ionizable cationic lipid in a total amount of 45-55 mol % of the total lipid content of the LNP composition.

### (III) PREPARING LIPID NANOPARTICLE (LNP) COMPOSITIONS

The method of making an LNP can comprise mixing a first solution with a second solution. Mixing can be achieved using standard liquid mixing techniques, such as propellor mixing, vortexing solutions or preferably through microfluidic mixing or high efficiency T-mixing. In some embodiments, the first solution comprises a lipid or a plurality of lipids and a nucleic acid, where all components are solubilized, in water/solvent system. The solvent may be any water miscible solvent (e.g., ethanol, methanol, isopropanol, acetonitrile, dimethylformamide, dimethylsulfoxide, dioxane or tetrahydrofuran). In some embodiments, the first solution comprises a small percentage of water or pH buffered water. The first solution may comprise up to at least 60% by volume of water, e.g., up to at least about 0.05%, 0.1%, 0.5%, 1%, 2%, 3%, 4%, 5%, 10%, 15%, 20%, 25%, 30%, 35%, 40%, 45%, 50%, 55% or 60% by volume of water. In an embodiment, the first solution comprises between about 0.05% and 60% by volume of water, e.g., between about 0.05% and 50%, about 0.05% and 40%, or about 5% and 20% by volume of water.

In some embodiments, the first solution comprises a single type of lipid, for example, an ionizable lipid, a phospholipid, a sterol, or a PEG-containing lipid. In some embodiments, the first solution comprises a plurality of lipids. In some embodiments, the plurality comprises an ionizable lipid, a phospholipid, a sterol, or a PEG-containing lipid. In some embodiments, the plurality of lipids comprise cholesterol, 1,2-distearoyl-sn-glycero-3-phosphocholine (DSPC), 1,2-dimyristoyl-rac-glycero-3-methylpolyoxyethylene2000 (DMG-PEG2k) or α-(3′-{[1,2-di(myristyloxy)propanoxy]carbonylamino}propyl)-ω-methoxy, polyoxyethylene (PEG2000-C-DMG), and an ionizable lipid. The plurality of lipids may exist in any ratio. In an embodiment, the plurality of lipids comprises an ionizable lipid or sterol, a phospholipid, a sterol, a PEG-containing lipid of the above lipids or a combination thereof in a particular ratio (e.g., a ratio described herein).

In some embodiments, the second solution is water. In some embodiments, the second solution is an aqueous buffer with a pH between 3-6 (e.g., a pH of about 3, about 4, about 5, or about 6). The second solution may comprise a load component, e.g., a nucleic acid (e.g., mRNA). The second solution may comprise a small percentage of water-miscible organic solvent. The second solution may comprise up to at least 60% by volume of at least one water miscible organic solvent, e.g., up to at least about 0.05%, 0.1%, 0.5%, 1%, 2%, 3%, 4%, 5%, 6%, 7% 8%, 9%, 10%, 15%, 20%, 25%, 30%, 35%, 40%, 45%, 50%, 55%, 60% or any percent therebetween by volume of at least one organic solvent (e.g., a water miscible organic solvent). In an embodiment, the second solution comprises between about 0.05% and 60% by volume of organic solvent, e.g., between about 0.05% and 50%, about 0.05% and 40%, or about 5% and 20% by volume of organic solvent (e.g., a water miscible organic solvent). The aqueous buffer solution can be an aqueous solution of citrate buffer. In some embodiments, the aqueous buffer solution is a citrate buffer solution with a pH between 4-6 (e.g., a pH of about 4, about 5, or about 6). In an embodiment, the aqueous buffer solution is a citrate buffer solution with a pH of about 6.

In some embodiments, the solution comprising a mixture of the first and second solutions comprising the LNP suspension can be diluted. In some embodiments, the pH of the solution comprising a mixture of the first and second solutions comprising the LNP suspension can be adjusted. Dilution or adjustment of the pH of the LNP suspension can be achieved with the addition of water, acid, base or aqueous buffer. In some embodiments, no dilution or adjustment of the pH of the LNP suspension is carried out. In some embodiments, both dilution and adjustment of the pH of the LNP suspension is carried out.

In some embodiments, excess reagents, solvents, unencapsulated nucleic acid maybe removed from the LNP suspension by tangential flow filtration (TFF) (e.g., diafiltration). The organic solvent (e.g., ethanol) and buffer may also be removed from the LNP suspension with TFF. In some embodiments, the LNP suspension is subjected to dialysis and not TFF. In some embodiments, the LNP suspension is subjected to TFF and not dialysis. In some embodiments, the LNP suspension is subjected to both dialysis and TFF.

In one aspect, the present disclosure features a method comprising treating a sample of LNPs comprising nucleic acid, with a fluid comprising a detergent (e.g., Triton X-100, or anionic detergents (such as, but not limited to, sodium dodecyl sulfate (SDS), or non-ionic detergent, such as but not limited to P-octylglucoside, or Zwittergent 3-14) for a period of time suitable to degrade the lipid layer and thereby release the encapsulated and/or entrapped nucleic acid(s). In an embodiment, the method further comprises analyzing the sample for the presence, absence, and/or amount of the released nucleic acid(s).

Some embodiments relate to the use of a salt form of the anionic phospholipid composition in the preparation of a liposomal nanoparticle (LNP) composition. In some embodiments, the use is in combination with one or more of the following LNP components during the preparation of the LNP composition: a TLR-activating nucleic acid; an ionizable cationic lipid (ICL); cholesterol; a (L-Serine) PS lipid; one or more phospholipids; and a conjugated lipid. In some embodiments, the use comprises the step of combining the ammonium or salt form of a compound of Formula (V-A-1) with one or more of the following LNP components during the preparation of the LNP composition: a TLR-activating nucleic acid; an ionizable cationic lipid (ICL); cholesterol; a (L-Serine) PS lipid; one or more phospholipids; and a conjugated lipid.

In some embodiments, a LNP composition is prepared using a sodium or ammonium salt of an anionic phospholipid. The salt form of the targeting lipid can influence its solubility in alcohol containing solvents used in the preparation of lipid nanoparticles. In some embodiments, certain salts of the phosphatidylserine targeting lipids are provided are useful in the preparation of the LNP compositions. For example, in some embodiments, the phosphatidylserine targeting lipids can be provided as an ammonium salt of DPPS having improved biophysical properties and higher solubility in the presence of ethanol, a preferred solvent for preparation of LNPs. The sodium salts of DSPS or DSPS were insoluble in ethanol and required both the presence of methanol and heating to allow for their formation, as did the ammonium salt of DSPS. It is contemplated that the other ammonium salts of phosphatidylserine will give rise to the same advantages in solubility and biophysical properties.

In some embodiments, a LNP composition is prepared using a sodium or ammonium salt of an anionic phospholipid. In some embodiments, the anionic phospholipid salt is a compound of Formula (V-A-1), having the chemical structure:

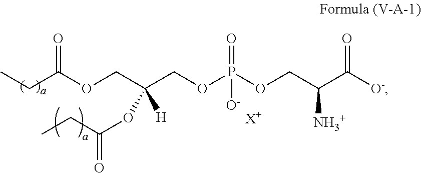

wherein
   X⁺ is an ammonium (NH₄⁺) or sodium (Na⁺) cation; and
   a is 14, 15 or 16.

In some embodiments, a is 14 or 16. In some embodiments, X⁺ is ammonium cation (NH₄⁺). In some embodiments, X⁺ is sodium cation (Na⁺) In some embodiments, X in Formula (V-A-1) is an ammonium cation selected from the group consisting of: ammonium (NH₄⁺), an alkylammonium, a di alkyl ammonium, and a tri alkyl ammonium salt. In some embodiments, X is X is an ammonium cation selected from the group consisting of: ammonium, dimethylamine, diethylamine, triethylamine, trimethylamine, 2-(dimethyamino)ethanol, diethanolamine, 2-(diethyamino)ethanol, ethanolamine, ethyl enedi amine, N-methyl-glucamine, imidazole, histidine, lysine, arginine, 4-(2-hydroxyethyl)-morpholine, piperazine, 1-(2-hydroxyethyl)-pyrrolidine, tri ethanol amine, and tromethamine (tris(hydroxymethyl)aminomethane).

In some embodiments, the anionic phospholipid of Formula (V-A-1) is a sodium salt of distearoylphosphatidyl-L-serine (DSPS L-isomer). In some embodiments, the anionic phospholipid of Formula (V-A-1) is an ammonium salt of distearoylphosphatidyl-L-serine (DSPS L-isomer). In some embodiments, the anionic phospholipid of Formula (V-A-1) is a sodium salt of DPPS (L-isomer). In some embodiments, the anionic phospholipid of Formula (V-A-1) is an ammonium salt of DPPS (L-isomer).

In some embodiments, the anionic phospholipid salt is selected from the group consisting of:

TABLE 4

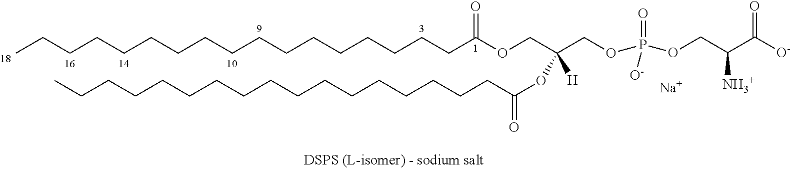 [SMILES: CCCCCCCCCCCCCCCCCC(=O)OC[C@H](COP(=O)([O-])OC[C@H]([NH3+])C(=O)[O-])OC(=O)CCCCCCCCCCCCCCCCC ; [Na+]]

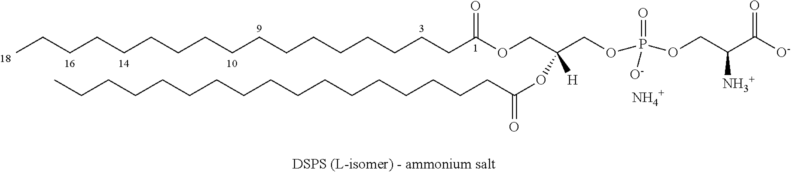 [SMILES: CCCCCCCCCCCCCCCCCC(=O)OC[C@H](COP(=O)([O-])OC[C@H]([NH3+])C(=O)[O-])OC(=O)CCCCCCCCCCCCCCCCC ; [NH4+]]

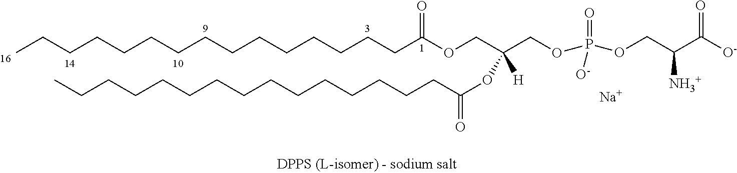 [SMILES: CCCCCCCCCCCCCCCC(=O)OC[C@H](COP(=O)([O-])OC[C@H]([NH3+])C(=O)[O-])OC(=O)CCCCCCCCCCCCCCC ; [Na+]]

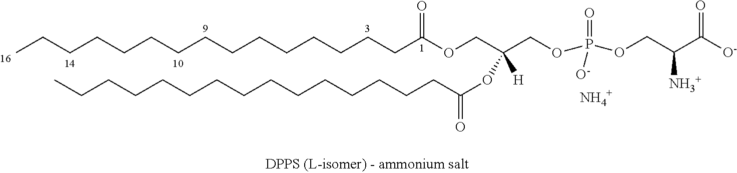 [SMILES: CCCCCCCCCCCCCCCC(=O)OC[C@H](COP(=O)([O-])OC[C@H]([NH3+])C(=O)[O-])OC(=O)CCCCCCCCCCCCCCC ; [NH4+]]

In some embodiments, the anionic phospholipid salt is DSPS (L-isomer) sodium salt. In some embodiments, the anionic phospholipid salt is DSPS (L-isomer) ammonium salt. In some embodiments, the anionic phospholipid salt is DPPS (L-isomer) sodium salt. In some embodiments, the anionic phospholipid salt is DPPS (L-isomer) ammonium salt. In some embodiments the targeting lipid is a sodium or ammonium salt of dipalmitoylphosphatidyl-L-serine (DPPS) or distearoylphosphatidyl-L-serine (DSPS). In some embodiments the targeting lipid is a sodium or ammonium salt of dipalmitoylphosphatidyl-L-serine (DPPS) or distearoylphosphatidyl-L-serine (DSPS).

In some embodiments, a LNP composition can comprise an anionic phospholipid selected from the group consisting of:

TABLE 5

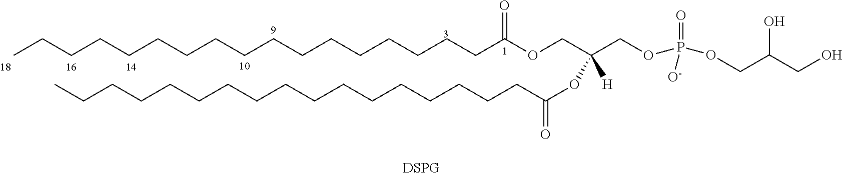 [SMILES: CCCCCCCCCCCCCCCCCC(=O)OC[C@H](COP(=O)([O-])OCC(O)CO)OC(=O)CCCCCCCCCCCCCCCCC]

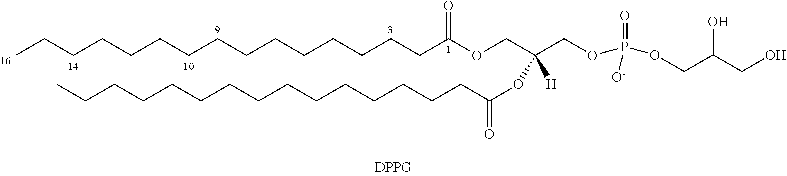 [SMILES: CCCCCCCCCCCCCCCC(=O)OC[C@H](COP(=O)([O-])OCC(O)CO)OC(=O)CCCCCCCCCCCCCCC]

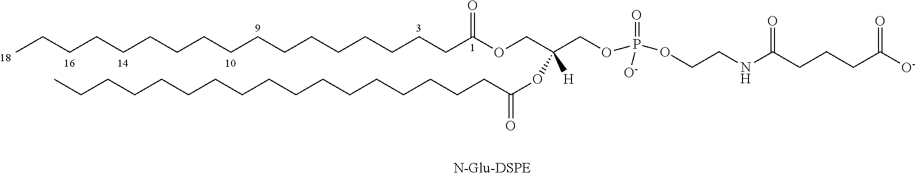 [SMILES: CCCCCCCCCCCCCCCCCC(=O)OC[C@H](COP(=O)([O-])OCCNC(=O)CCCC(=O)[O-])OC(=O)CCCCCCCCCCCCCCCCC]

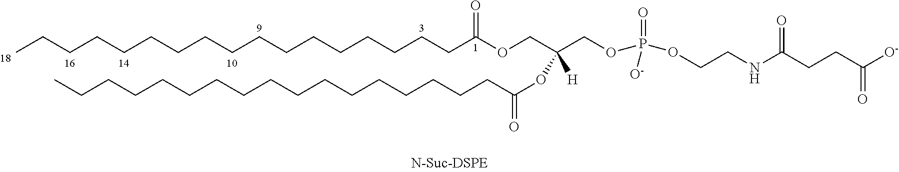 [SMILES: CCCCCCCCCCCCCCCCCC(=O)OC[C@H](COP(=O)([O-])OCCNC(=O)CCC(=O)[O-])OC(=O)CCCCCCCCCCCCCCCCC]

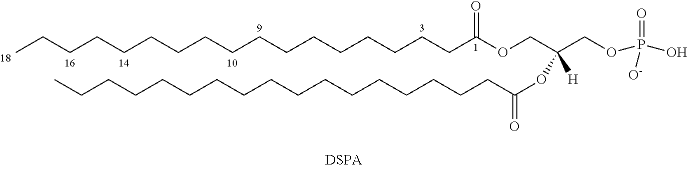 [SMILES: CCCCCCCCCCCCCCCCCC(=O)OC[C@H](COP(=O)([O-])O)OC(=O)CCCCCCCCCCCCCCCCC]

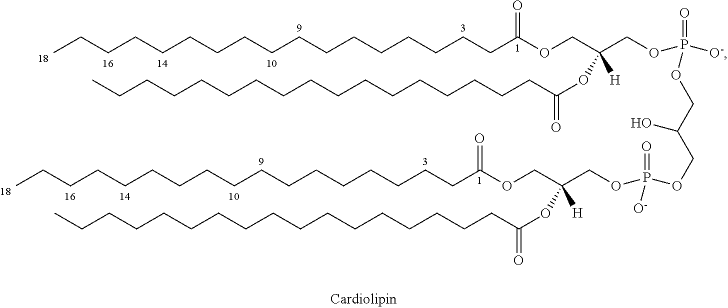

or an ammonium or sodium salt thereof.

In some embodiments, the salt form of phosphatidylserine is highly soluble in ethanol. In some embodiments it is soluble at greater than 0.5 mg/ml, greater than 1 mg/mL, greater than 5 mg/mL, greater than 10 mg/mL, or greater than 20 mg/mL. In some embodiments, the salt is an ammonium salt. In some embodiments, the salt is ammonium itself, an alkylammonium, a dialkylammonium, or a trialkylammonium salt. In some embodiments, the amine is chosen from ammonia, dimethylamine, diethylamine, triethylamine, trimethylamine, 2-(dimethyamino)ethanol, diethanolamine, 2-(diethyamino)ethanol, ethanolamine, ethylenediamine, N-methyl-glucamine, imidazole, histidine, lysine, arginine, 4-(2-hydroxyethyl)-morpholine, piperazine, 1-(2-hydroxyethyl)-pyrrolidine, triethanolamine, and tromethamine (tris(hydroxymethyl)aminomethane), In some embodiments, this targeting lipid is an ammonium salt of DPPS.

In some embodiments, the salt form of phosphatidylglycerol or phosphatidylserine is highly soluble in ethanol. In some embodiments, the salt form of phosphatidylserine is highly soluble in ethanol. In some embodiments it is soluble at greater than 0.5 mg/ml, greater than 1 mg/mL, greater than 5 mg/mL, greater than 10 mg/mL, or greater than 20 mg/mL. In some embodiments the salt form of phosphatidylglycerol or phosphatidylserine is soluble is at least 0.3 mM, at least 0.4 mM, at least 0.5 mM, at least 0.6 mM, or at least 0.8 mM, as determined by a shake flask method in 200 proof ethanol, at the temperature of 22° C. of less. In some embodiments, the salt is an ammonium salt. In some embodiments, the phosphatidylserine is added to the LNP lipids in the form of ammonium or a substituted ammonium salt. Substituted ammonium salt can be mono-, di-. tri-, or tetraalkylammonium having alkyl groups with one to six, one to four, one to three, one, two, or three carbon atoms each. One or more alkyl groups can be n-alkyl, or branched alkyl groups (such as, for example, isopropyl groups), or form a ring (such as for example, cyclohexyl group). An alkyl group and the nitrogen ammonium atom may form a heterocyclic ring. The substituted ammonium salt may be also formed by an alkylenediamine. Tris(hydroxymethyl)aminomethane and triethanolamine can also be used as the amine bases to form PS salts. In some embodiments, the amine is chosen from ammonia, dimethylamine, diethylamine, triethylamine, trimethylamine, 2-(dimethyamino)ethanol, diethanolamine, 2-(diethyamino)ethanol, ethanolamine, ethylenediamine, N-methyl-glucamine, imidazole, histidine, lysine, arginine, 4-(2-hydroxyethyl)-morpholine, piperazine, 1-(2-hydroxyethyl)-pyrrolidine, triethanolamine, and tromethamine (tris(hydroxymethyl)aminomethane), In some embodiments, this targeting lipid is an ammonium salt of DPPS.

TABLE 6
Ammonium and sodium salt forms of dipalmitoyl- or distearoyl-phosphatidylserine.

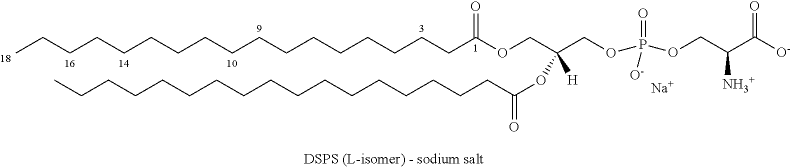 [SMILES: CCCCCCCCCCCCCCCCCC(=O)OC[C@H](COP(=O)([O-])OC[C@H]([NH3+])C(=O)[O-])OC(=O)CCCCCCCCCCCCCCCCC ; [Na+]]

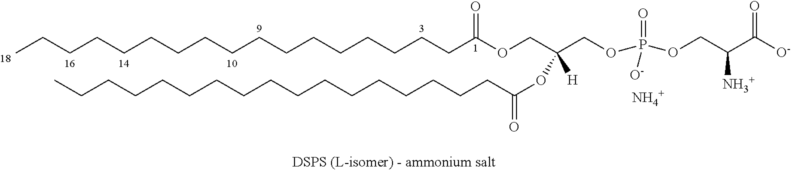 [SMILES: CCCCCCCCCCCCCCCCCC(=O)OC[C@H](COP(=O)([O-])OC[C@H]([NH3+])C(=O)[O-])OC(=O)CCCCCCCCCCCCCCCCC ; [NH4+]]

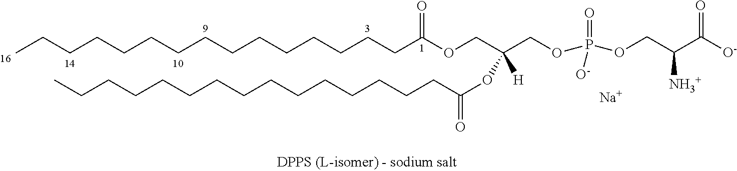 [SMILES: CCCCCCCCCCCCCCCC(=O)OC[C@H](COP(=O)([O-])OC[C@H]([NH3+])C(=O)[O-])OC(=O)CCCCCCCCCCCCCCC ; [Na+]]

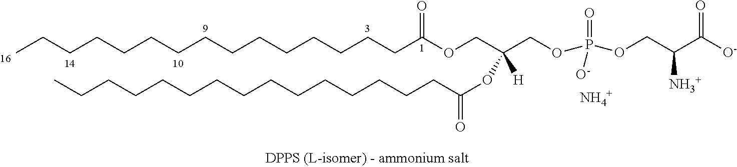 [SMILES: CCCCCCCCCCCCCCCC(=O)OC[C@H](COP(=O)([O-])OC[C@H]([NH3+])C(=O)[O-])OC(=O)CCCCCCCCCCCCCCC ; [NH4+]]

To obtain phosphatidylserine in the form of ammonium or substituted ammonium salt, any method known in the art may be used. In some embodiments, a sodium salt of phosphatidylserine (PS) is dissolved in a monophase system of chloroform, methanol, and water, containing a chloride salt of ammonium or substituted ammonium (a Bligh-Dyer monophase), and the system is brought to the two-phase state by adding extra methanol and/or water containing the ammonium or substituted ammonium chloride. The chloroform-rich phase, containing the PS, is separated, and the process is repeated. Finally, the chloroform-rich phase is washed with water to remove excess chloride, and the ammonium (substituted ammonium) salt of PS is obtained by evaporation of the chloroform-rich phase. Optionally, the obtained ammonium or substituted ammonium salt of PS is vacuum dried or dissolved in cyclohexane and lyophilized. In another embodiment, the PS as a sodium or potassium salt is dissolved in a water-immiscible organic solvent, such as chloroform or a chloroform-methanol mixture, and washed with diluted aqueous solution of an acid, such as HCl, to obtain a free acid form of the PS, which is then neutralized with ammonium hydroxide or substituted amine in free base form. In yet another embodiment, the organic solution of PS as a sodium or potassium salt is treated with a cation-exchange resin in the ammonium of substituted ammonium form. In yet another embodiment, the PS is prepared in the form of a calcium or magnesium salt and treated with ammonium or substituted ammonium salt of a chelator, such as EDTA, or with ammonium or substituted ammonium phosphate, in the presence of an organic solvent, causing displacement of calcium or magnesium ion in the form of a chelate or a yet less soluble phosphate, which is separated, e.g., by filtration, while ammonium or substituted ammonium salt of PS is left in the organic (e.g., ethanol) solution.

In some embodiments, PS or PG are added to the LNP lipid formulation at a concentration between about 0.1 mol % to about 20 mol %, about 0.1 mol % to about 10 mol %, about 0.1 mol % to about 5 mol %, about 0.5 mol % to about 20 mol %, about 0.5 mol % to about 10 mol %, about 0.5 mol % to about 5 mol %, about 1 mol % to about 20 mol %, about 1 mol % to about 10 mol %, or about 1 mol % to about 5 mol %, of the total lipid content of the LNP. In some embodiments, the PS is added to the LNP lipid formulation at a concentration between about 1 mol % to about 20 mol %, about 2.5 mol % to about 10 mol %, about 3 mol % to about 9 mol %, or about 4 mol % to about 8 mol %, of the total lipid content of the LNP.

In some embodiments, the PS or PG lipid is included in the LNP composition comprising ionizable cationic lipids known in the art, including DODAP, DODMA, AKG-OA-DM2, O-11769, DLin-MC3-DMA, DLin-KC2-DMA, DLin-KC3-DMA, ALC-0315, and SM-102.

In another embodiment the PS lipid is included in the LNP composition comprising ICLs of Formula I, II, III, IV-B, V-A-1, combinations thereof or pharmaceutically salts thereof. In another embodiment the PS lipid is included in the LNP composition using N/P ratios between 3 and 8, between 4 and 7, or between 5 and 6.

The TLR agonist may be formulated with the LNP composition in any manner known in the art. In some embodiments, the agonist is simply admixed in the LNP-containing formulation in the form of a solution. An effective amount of the agonist in the form of a stock solution in a suitable solvent (e.g., water or aqueous buffer) or as a neat solid is added to the LNPs in a physiologically acceptable medium and mixed to achieve dissolution.

In some embodiments, the agonist is encapsulated in the LNP. Encapsulation of the agonist is achieved by any method known in the art, including, without limitation, addition of the agonist to the aqueous solution, optionally comprising a nucleic acid component, and/or in the organic solvent solution comprising the LNP lipids, and combining them to effect formation of LNPs, as described herein in the section “Making Lipid Nanoparticles”. In one embodiment the combining is in a flow-through fluid mixer. The nucleic acid may comprise a TLR-activating nucleic acid, an mRNA nucleic acid, a DNA nucleic acid, or any combination of the foregoing. The fluid mixer may comprise a T- or Y-shaped fluid junction, optionally with a microfluidic pattern providing for a desired in-flow mixing characteristics (flow rate, shear forces, turbulent or lamellar flow). Alternatively, LNPs can be formed using any method known for production of liposomes, such as lipid film hydration or solvent injection, followed by extrusion through track-etched (for example, polycarbonate) membranes with defined pore size, and the encapsulation of a TLR agonist is by direct entrapment, by hydrophobic capture in the bilayer membrane, or by transmembrane gradient methods known in the art (Lasic D., 1993, Liposomes: From Physics to Applications, Elsevier, 567 p.; Drummond et al, 1999, Pharm Rev. 51:691-744; Kirpotin et al., 2012, Meth. Enzymol., 502:139-166).

In some embodiments, the agonist has characteristics of a lipid, such as TLR4 agonist monophosphoryl Lipid A, disclosed in more detail below, and can be used for LNP encapsulation without further modification. In some embodiments, the agonist is prepared in the form of a conjugate with a lipophilic moiety such as a lipid molecule. Lipophilic conjugates of TLR agonists, for example, telratolimod (3M-052; U.S. Pat. No. 9,242,980; Smirnov et al. 2011, Vaccine, 29(33), 5434-5442), and 1V270 (TMX-201; Chan et al. 2009, Bioconjugate Chem., 20:1194-1200; Wu et al., 2014, J Innate Immun. 6:315-324) are known in the art. In some embodiments, the agonist-lipid conjugate is encapsulated into LNP by adding it to the lipid solution prior to contacting of the lipids with the aqueous medium. If the agonist-lipid conjugate is sufficiently soluble in aqueous media, it can be added to the aqueous medium further combined with the LNP lipids. In some embodiments the lipophilic moiety is a lipid-hydrophilic polymer conjugate, and the agonist molecule is conjugated to the terminal region of the polymer moiety contralateral to the lipid. The conjugate is prepared by the reaction of a terminally activated lipid-polymer conjugate with the reactive group introduced in the structure of the agonist. The suitable conjugation chemistry is well known in the art (Hermanson G., Bioconjugate Techniques, 3^{rd} Ed., Academic Press, 2013).

In some embodiments, the lipid-hydrophilic polymer conjugate is a PEG-lipid. The molecular weight of the PEG moiety is selected to provide for sufficient aqueous solubility of the agonist-PEG-lipid conjugate. In some embodiments, the PEG molecular weight of the PEG moiety is between 500-10,000 Da, between 1,000-10,000 Da, or between 2,000-5,000 Da. In some embodiments, the lipid moiety of the conjugate comprises one or two of the C₁₂-C₁₈ hydrocarbon chains. In some embodiments, the lipid moiety of the conjugate comprises conjugated diacylglycerol or diacylphosphatidylethanolamine wherein the acyl is C12:0 (lauroyl), C14:0 (myristoyl), C16:0 (palmitoyl), C18:0 (stearoyl), or C18:1(9Z) (oleoyl). When dissolved in an aqueous medium, the agonist-polymer-lipid conjugate typically forms micelles. In some embodiments, an effective amount of the agonist-polymer-lipid-conjugate micellar solution is simply admixed to the pre-formed LNP formulation. In some embodiments, the agonist-polymer-lipid conjugate micelles are contacted with the pre-formed LNPs, optionally comprising a nucleic acid, under the conditions that allow the lipid part of the conjugate to “insert” itself in the outer lipid layer of the LNP and become bound to the LNP surface via a polymer spacer. In this way, the agonist moiety of the conjugate is advantageously exposed on the surface of the LNP and available for unimpeded interaction with the TLR receptors located on the inner surface of endocytic vesicles upon endocytosis of the LNP. Especially advantageous are LNP compositions that comprise both an agonist-polymer-lipid ligand and a ligand that promotes endocytosis of the LNP, such as, without limitation, L-phosphatidylserine (L-PS), or phosphatidylglycerol (PG), in the LNP composition, as described in more detail herein.

In some embodiments, after encapsulation, the unencapsulated agonist or agonist conjugate is removed from the LNP formulation. Removing unencapsulated agonist or agonist conjugate from the LNP formulation can be achieved, without limitation, by the methods of dialysis, adsorption, ion exchange, ultrafiltration, or diafiltration, including tangential flow filtration.

The agonist may be co-encapsulated with the nucleic acid within the same LNP, or they can be separately encapsulated, and the resulting agonist LNP composition and nucleic acid LNP composition are mixed to achieve effective concentrations of both the agonist and the nucleic acid. In some embodiments, the amount of a small molecule TLR agonist-lipid conjugate or agonist-polymer-lipid conjugate in the LNP composition lies in the range of 0.05-10 mol %, 0.1-10 mol %. 0.1-5 ml %, 0.1-2 mol %, or 0.1-1 mol % of the total lipid.

In some embodiments, the composition further comprises a pharmaceutical excipient.

In some embodiments, the lipidic nanoparticles are in an aqueous medium.

In some embodiments, the nucleic acid is entrapped in the lipidic nanoparticle with an ionizable cationic lipid compound provided herein or combinations thereof, wherein the nucleic acid is either RNA or DNA. In some embodiments, the nucleic acid is mRNA. In some embodiments, the nucleic acid is siRNA. In some embodiments, the nucleic acid is DNA.

In some embodiments, the lipidic nanoparticle comprises a membrane comprising phosphatidylcholine and a sterol. In some embodiments, the sterol is cholesterol. In some embodiments, the lipidic nanoparticle comprises a membrane comprising phosphatidylcholine, ionizable cationic lipid (ICL). In some embodiments, the ICL have a structure of Formula I, and cholesterol, wherein the membrane separates the inside of the lipidic nanoparticles from the aqueous medium. In some embodiment, the ICL have a structure as shown in Table 1A. In some embodiments, the phosphatidylcholine is distearoylphosphatidylcholine (DSPC) or hydrogenated soy phosphatidylcholine (HSPC). In some embodiments, the ionizable cationic lipid to cholesterol molar ratios is from about 65:35 to 40:60. In some embodiments, the ICL to cholesterol molar ratio is from about 60:40 to about 45:55. In some embodiments, the phosphatidylcholine to cholesterol molar ratio is from about 1:5 to about 1:2. In some embodiments, the membrane further comprises a polymer-conjugated lipid. In some embodiments, the lipidic nanoparticle comprises ICL, DSPC, cholesterol and polymer-conjugated lipid in a about 49.5:10.3:39.6:2.5 molar ratio.

In some embodiments, the polymer-conjugated lipid is PEG(2000)-dimyristoylglycerol (PEG-DMG) or PEG(Mol. weight 2,000)-dimyristoylphosphatidylethanolamine (PEG-DMPE).

### (IV) PHARMACEUTICAL COMPOSITIONS

The compositions of this disclosure may be administered by various routes, for example, to effect systemic delivery via intravenous, parenteral, intraperitoneal, or topical routes. The compositions may be administered intravenously, subcutaneously, or intraperitoneally to a subject.

In some embodiments, the composition is a liquid pharmaceutical formulation for parenteral administration. In some embodiments, the composition is a liquid pharmaceutical formulation for subcutaneous, intramuscular, or intradermal administration. In some embodiments, the composition is in the form of a lyophilized powder, that is subsequently reconstituted with aqueous medium prior to administration.

In some embodiments, an LNP described herein has a diameter between 5 and 500 nm, e.g., between 10 and 400 nm, 20 and 350 nm, 25 and 325 nm, 30 and 300 nm, 50 and 250 nm, 60 and 200 nm, 75 and 190 nm, 80 and 180 nm, 100 and 200 nm, 200 and 300 nm, and 150 and 250 nm. The diameter of an LNP may be determined by any method known in the art, for example, dynamic light scattering, transmission electron microscopy (TEM) or scanning electron microscopy (SEM). In some embodiments, an LNP has a diameter between 50 and 100 nm, between 70 and 100 nm, and between 80 and 100 nm. In an embodiment, an LNP has a diameter of about 90 nm. In some embodiments, an LNP described herein has a diameter greater than about nm. In some embodiments, an LNP has a diameter greater than about 35 nm, about 40 nm, about 45 nm, about 50 nm, about 60 nm, about 70 nm, about 80 nm, about 90 nm, about 100 nm, about 120 nm, about 140 nm, about 160 nm, about 180 nm, about 200 nm, about 225 nm, about 250 nm, about 275 nm or about 300 nm. In an embodiment, an LNP has a diameter greater than about 70 nm. In an embodiment, an LNP has a diameter greater than about 90 nm. In an embodiment, an LNP has a diameter greater than about 180 nm.

In some embodiments, a plurality of LNPs described herein has an average diameter ranging from about 40 nm to about 180 nm. In some embodiments, a plurality of LNPs described herein has an average diameter from about 50 nm to about 150 nm. In some embodiments, a plurality of LNPs described herein has an average diameter from about 50 nm to about 120 nm.

In some embodiments, a plurality of LNPs described herein has an average diameter from about 60 nm to about 120 nm. In some embodiments, a plurality of LNPs has an average diameter of about 40 nm, about 45 nm, about 50 nm, about 60 nm, about 70 nm, about 80 nm, about 90 nm, about 100 nm, about 120 nm, about 140 nm, about 160 nm, about 180 nm.

In some embodiments, a nanoparticle or plurality of nanoparticles described herein has an average neutral to negative surface charge of less than −100 mv, for example, less than −90 mv, −80 mv, −70 mv, −60 mv, −50 mv, −40 mv, −30 mv, and −20 mv. In some embodiments, a nanoparticle or plurality of nanoparticles has a neutral to negative surface charge of between −100 my and 100 my, between −75 my to 0, or between −50 my and −10 mv.

In some embodiments, at least 5% (e.g., at least 10%, at least 15%, at least 20%, at least 25%, at least 30%, at least 40%, at least 50%, at least 60%, at least 70%, at least 80%, at least 90%, at least 95%, or at least 99%) of the nanoparticles of a plurality of nanoparticles have an average neutral to negative surface charge of less than −100 mv. In some embodiments, a nanoparticle or plurality of nanoparticles has an average surface charge of between −20 my to +20, between −10 my and +10 mv, or between −5 my and +5 my at pH 7.4. LNPs that are neutral in charge have improved pharmacokinetics and biological performance compared to cationic LNPs.

### (V) USE OF LIPOSOMAL NANOPARTICLE (LNP) COMPOSITIONS

In some aspects, a method of delivering a nucleic acid to a cell is provided, the method comprising: contacting the cell with a composition comprising an LNP comprising a ligand (also referred herein as targeting ligand) having a binding specificity for a cell surface antigen, wherein the binding of the ligand to the antigen induces the internalization of the ligand. In some embodiments, the targeting ligand can be, but is not limited to, an internalizing antibody, or a fragment thereof, a small molecule conjugates or gylcoconjugates. In some embodiments, the binding of the targeting ligand to a specific cell surface antigen induces the internalization of the LNP with the targeting ligand attached by a cell expressing at least 100,000 or at least 1,000,000 molecules of the antigen when contacted and incubated with the cell under internalizing conditions.

According to some embodiments, a proportion of LNPs are taken up DC cells, while most will accumulate in the liver and spleen. The DC cells can express the antigenic peptide, process it for MHC I presentation and travel to the lymph node for presentation to naïve T cells inducing an education of memory T-cells towards the antigen.

The main types of professional antigen—presenting cells are dendritic cells, which have the broadest range of antigen presentation, and are probably the most important antigen—presenting cells, macrophages, B-cells, and certain activated epithelial cells. Dendritic cells (DCs) are leukocyte populations that present antigens captured in peripheral tissues to T cells via both MHC class II and I antigen presentation pathways. It is well known that dendritic cells are potent inducers of immune responses and the activation of these cells is a critical step for the induction of antitumoral immunity. Dendritic cells are conveniently categorized as “immature” and “mature” cells, which can be used as a simple way to discriminate between two well characterized phenotypes.

However, this nomenclature should not be construed to exclude all possible intermediate stages of differentiation. Immature dendritic cells are characterized as antigen presenting cells with a high capacity for antigen uptake and processing, which correlates with the high expression of Fcγ receptor and mannose receptor. The mature phenotype is typically characterized by a lower expression of these markers, but a high expression of cell surface molecules responsible for T cell activation such as class I and class II MHC, adhesion molecules (e.g. CD54 and CD11) and costimulatory molecules (e.g., CD40, CD80, CD86 and 4-1 BB1). Dendritic cell maturation is referred to as the status of dendritic cell activation at which such antigen—presenting dendritic cells lead to T cell priming, while presentation by immature dendritic cells results in tolerance. Dendritic cell maturation is chiefly caused by biomolecules with microbial features detected by innate receptors (bacterial DNA, viral RNA, endotoxin, etc), pro-inflammatory cytokines (TNF, IL-1, IFNs), ligation of CD40 on the dendritic cell surface by CD4OL, and substances released from cells undergoing stressful cell death. The dendritic cells can be derived by culturing bone marrow cells in vitro with cytokines, such as granulocyte-macrophage colony-stimulating factor (GM CSF) and tumor necrosis factor alpha. Non-professional antigen-presenting cells do not constitutively express the MHC class II proteins required for interaction with naive T cells; these are expressed only upon stimulation of the non-professional antigen-presenting cells by certain cytokines such as IFNγ. “Antigen presenting cells” can be loaded with MHC class I presented peptides by transducing the cells with nucleic acid, preferably mRNA, encoding a peptide or polypeptide comprising the peptide to be presented, e.g. a nucleic acid encoding the antigen.

In some embodiments, a pharmaceutical composition comprising a gene delivery vehicle that targets a dendritic or other antigen presenting cell is administered to a patient, resulting in transfection that occurs in vivo. In some embodiments, methods of targeting nucleic acid expression to dendritic cells comprises contacting a dendritic cell with a LNP composition disclosed herein. Dendritic cells (DCs) are specialized antigen-presenting cells that play a central role in initiating and regulating adaptive immunity. Owing to their potent antigen (Ag) presentation capacity and ability to generate distinct T-cell responses, efficient and specific delivery of Ags to DCs is the cornerstone for generating Ag-specific effector and memory cells against tumors or pathogens. Dendritic cells can be generated from human blood monocytes by adding granulocyte-macrophage colony-stimulating factor (GM-CSF), IL-4, and IFN-gamma to differentiate monocyte-derived DC in vitro. Cells in culture exhibit both dendritic and veiled morphologies, the former being adherent, and the latter suspended. Phenotypically, they are CD1a−/dim, CD11a+, CD11b++, CD11c+, CD14dim/−, CD16a−/dim, CD18+, CD32dim/−, CD33+, CD40+, CD45R0+, CD50+, CD54+, CD64−/dim, CD68+, CD71+, CD80dim, CD86+/++, MHC class I++/, HLA-DR++/, HLA-DP+, and HLA-DQ (Geiseler et al. Dev Immunol. 1998; 6(1-2):25-39). Alternatively, human primary blood dendritic cell lines have been developed and are commercially available from Creative Biolabs. In some embodiments, LNPs are added to cultured human dendritic cells at an appropriate concentration, (e.g. 1-5 μg/mL mRNA). After some time to allow for cellular uptake and antigen expression, human T cells (HemaCare) can be added, and the cell culture media is sampled at various times for INF-7 by Elisa (R&D Systems, DIF50C). Alternatively, the cells can be analyzed by flow cytometry for CD8+ marker or intracellular INFγ production (PE anti-human IFN-γ antibody, Biolegend).

In some embodiments, methods of targeting nucleic acid expression of epitopes for CD8 cells are provided comprising the use or administration of a LNP composition comprising a nucleic acid encoding a CD8 epitope. CD8+ T cells can produce IL2, IFN-γ, and TNF, cytokines that are known to have critical functions during Mycobacterium tuberculosis infection. Importantly, CD8+ T cells have cytolytic functions to kill Mycobacterium tuberculosis-infected cells via granule-mediated function (via perforin, granzymes, and granulysin) or Fas-Fas ligand interaction to induce apoptosis. In humans, CD8+ T cell can produce granulysin, which can kill Mycobacterium tuberculosis directly. Therefore, it is anticipated that antigen generating mRNA LNPs delivered to DC will stimulate a CD8+ T cell response to fight against Mycobacterium tuberculosis infection. CD8+ T cells are able to recognize M. tuberculosis specific antigens (as peptides) presented by classical and non-classical MHC molecules. Classically restricted CD8+ T cells have been identified that recognize antigens presented by antigen presenting cells in the context of classical MHC Ia (HLA-A, -B, -C) molecules. Non-classically restricted CD8+ T cells include those CD8+ T cells that are capable of recognizing Mg antigen in the context of HLA-E molecules (non-MHC 1a), glycolipids associated with group 1 CD1 molecules and MHC I-related molecules (MR1) such as mucosal associated invariant T cells (MAIT). Finally, γδ T cells represent a separate population of CD8 (and CD4) T cells that have both innate and adaptive functions in response to Mycobacterium tuberculosis infection. CD8+ T cells have been shown to play direct functions in response to Mycobacterium tuberculosis infection but they also play important roles in orchestrating many different functions in the overall host immune response (e.g., interaction to provide optimal CD4 T cell function).

In some embodiments, LNPs that have been modified with a targeting ligand such as phosphatidylserine are administered into a subject at a dose of about 1 μg to about 500 μg mRNA. In other embodiments, the targeting ligand is phosphatidylglycerol. In some embodiments the targeted LNPs are administered at a reduced dose of about 1 μg to about 100 ug mRNA. According to some embodiments, a higher proportion of LNPs can be taken up DC cells, allowing for increased production of antigenic peptide compared to non-targeted LNP and a more efficient vaccination against the pathogen. For example, assessing the CD8+ reactivity to the in vivo produced antigen could be accomplished by measuring INFγ plasma levels by species specific IFN-gamma Quantikine ELISA Kits from R&D Systems.

In some embodiments, the disclosure provides methods for in vivo delivery of nucleic acids to a subject. In some embodiments, the composition is administered subcutaneously, intramuscularly, or intradermally. In some embodiments, the lipidic nanoparticle is administered parenterally. In some embodiments the lipid nanoparticle is administered intramuscularly (IM). In general, administration to a patient is by intradermal injection is possible. However, injection may also be carried out intranodally into a lymph node (Maloy et al. (2001), Proc Natl Acad Sci USA 98:3299-3033). The resulting cells present the complex of interest and are recognized by autologous cytotoxic T lymphocytes which then propagate. In some embodiments, the composition is administered by inhalation. In some embodiments, the composition is formulated as nasal spray, and/or aerosol.

In some embodiments, the lipidic nanoparticle composition is administered as part of a single injection. In some embodiments the lipid nanoparticle is administered in multiple injections spaced in time to optimize the T-cell response to them.

Disclosed herein are compounds, compositions and methods related to the treatment of infections, such as mycobacterial infections. In some embodiments, the bacterial infection is Mycobacterium tuberculosis infection. In some embodiments, the bacterial infection is a form of nontuberculosis mycobacterium.

Aspects of the disclosure relate to a method of preventing a bacterial or viral infection, the method comprising administering to a subject in need thereof an effective amount of the composition provided herein to elicit an immune response. Aspects of the disclosure provide methods of vaccinating a subject comprising administering to the subject a single dosage of the compositions described herein comprising a nucleic acid (e.g. mRNA) encoding a polypeptide in an effective amount to vaccinate the subject. In some embodiments, the nucleic acid is formulated within a cationic lipidic nanoparticle. In some embodiments, the lipidic nanoparticle composition is administered as a single injection.

Other aspects of the disclosure relate to the use of these ionizable lipids or lipidic nanoparticles compositions comprising ionizable lipids in vaccines for the prevention of infectious diseases. Disclosed herein are methods for preventing mycobacteria infection, such as Mycobacterium tuberculosis. Additional mycobacteria include, but are not limited to, Mycobacterium avium complex, Mycobacterium leprae, Mycobacterium gordonae, Mycobacterium abscessus, Mycobacterium abscessus, Mycobacterium mucogenicum, and Mycobacterium. Other aspects of the disclosure relate to a method of preventing a bacterial or viral infection, the method comprising administering to a subject in need thereof an effective amount of the composition provided herein to elicit an immune response. Some embodiments provide methods of vaccinating a subject in need thereof, the method comprising administering the composition comprising a nucleic acid encoding an antigenic protein. In some embodiments, the compositions described herein can be used to prevent infections related to tuberculosis. In some embodiments, the vaccine is used for the prevention mycobacterium infections. In some embodiments, the vaccine can be used for the prevention of tuberculosis, nontuberculous mycobacteria (NTM), nontuberculosis lung disease, leprosy, Mycobacterium avium-intracellulare, mycobacterium kansasii, mycobacterium marinum, mycobacterium ulcerans, mycobacterium chelonae, mycobacterium fortuitum, or Mycobacterium abscessus.

Administration of a vaccine for inducing a second immune response may provide MHC class II-presented epitopes that are capable of eliciting a CD4+ helper T cell response against cells expressing antigens from which the MHC presented epitopes are derived. Alternatively or additionally, administration of a vaccine for inducing a second immune response may provide MHC class I-presented epitopes that are capable of eliciting a CD8+ T cell response against cells expressing antigens from which the MHC presented epitopes are derived. Furthermore, administration of a vaccine for inducing a second immune response may provide one or more neo epitopes (including known neo epitopes) as well as one or more epitopes not containing cancer specific somatic mutations but being expressed by cancer cells and preferably inducing an immune response against cancer cells, preferably a cancer specific immune response. In some embodiments, administration of a vaccine for inducing a second immune response provides neo-epitopes that are MHC class II-presented epitopes and/or are capable of eliciting a CD4+ helper T cell response against cells expressing antigens from which the MHC presented epitopes are derived as well as epitopes not containing cancer-specific somatic mutations that are MHC class I-presented epitopes and/or are capable of eliciting a CD8+ T cell response against cells expressing antigens from which the MHC presented epitopes are derived. In some embodiments, the epitopes do not contain cancer-specific somatic mutations.

Actual dosage levels of the active agents in the pharmaceutical compositions disclosed herein may be varied so as to obtain an amount of the active agent which is effective to achieve the desired therapeutic response for a particular patient, composition, and mode of administration, without being toxic to the patient. Dosage regimens can be adjusted to provide the optimum desired response (e.g., a therapeutic response). For example, one or more doses may be administered over time or the dose may be proportionally reduced or increased as indicated by the exigencies of the therapeutic situation. In some embodiments, LNPs are administered into a subject at a dose of about 0.01 to about 5 mg/kg mRNA by any route of administration known in the art and/or outlined above.

In some embodiments, the dose comprises between 0.01 to 5 mg/kg of nucleic acid. In some embodiments, the dose comprises between 0.01 to 5 mg/kg of mRNA. In some embodiments, the dose comprises between 0.01 to 3 mg/kg of nucleic acid. In some embodiments, the dose comprises between 0.01 to 3 mg/kg of mRNA. In some embodiments, the dose comprises between 0.01 to 1 mg/kg of nucleic acid. In some embodiments, the dose comprises between 0.01 to 1 mg/kg of mRNA. In some embodiments, the dose comprises between 0.01 to 0.5 mg/kg of nucleic acid. In some embodiments, the dose comprises between 0.01 to 0.5 mg/kg of mRNA. In some embodiments, the dose comprises between 0.01 to 1 mg/kg of mRNA. In some embodiments, the dose comprises between 0.01 to 0.1 mg/kg of nucleic acid. In some embodiments, the dose comprises between 0.01 to 0.05 mg/kg of mRNA. In some embodiments, the dose comprises between 0.01 to 0.1 mg/kg of nucleic acid. In some embodiments, the dose comprises between 0.01 to 0.05 mg/kg of mRNA.

The dosage of the compounds and/or of their pharmaceutically acceptable salts or the LNPs comprising the compounds and/or of their pharmaceutically acceptable salts may vary within wide limits and should naturally be adjusted, in each particular case, to the individual conditions and to the pathogenic agent to be controlled.

### (VI) ADDITIONAL EMBODIMENTS

Provided in some aspect of the disclosure is a TLR-activating nucleic acid lipid nanoparticle (LNP) composition comprising: a nucleic acid having at least 90% identity (e.g. 90%, 91%, 92%, 93%, 94%, 95%, 96%, 97%, 98%, 99% or more) with a nucleic acid sequence set forth herein; a KC3 ionizable cationic lipid in a total amount of 40-65 mol % of the total lipid content of the LNP composition; cholesterol in a total amount of 23.5-43.5 mol % of the total lipid content of the LNP composition; a (L-Serine) PS lipid in a total amount of 2.5-10 mol % of the total lipid content of the LNP composition; DSPC or HSPC phospholipid in a total amount of 5-25 mol % of the total lipid content of the LNP composition; and a PEG-containing conjugated lipid in a total amount of 0.5 mol % to 2.5 mol % of the total lipid content of the LNP composition.

Aspects of the disclosure relate to a TLR-activating nucleic acid lipid nanoparticle (LNP) composition comprising: a TLR-activating nucleic acid; an ionizable cationic lipid in a total amount of 40-65 mol % of the total lipid content of the LNP composition; cholesterol in a total amount of 23.5-43.5 mol % of the total lipid content of the LNP composition; a (L-Serine) PS lipid in a total amount of 2.5-10 mol % of the total lipid content of the LNP composition; DSPC or HSPC phospholipid in a total amount of 5-25 mol % of the total lipid content of the LNP composition; and a PEG-containing conjugated lipid in a total amount of 0.5 mol % to 2.5 mol % of the total lipid content of the LNP composition. In some embodiments, the TLR-activating nucleic acid is an RNA oligonucleotide. In some embodiments, the TLR-activating nucleic acid is an DNA oligonucleotide. In some embodiments, the N/P ratio is 3 to 8. In some embodiments, the ionizable lipid is selected from AKG-OA-DM2, AKG-UO1, DODMA, DODAP, AKG-OA-DM-3, KC3-OA, KC3-01, and KC3-PA. In some embodiments, the ionizable cationic lipid is selected from the group consisting of: KC3-OA, KC3-PA, KC3-C17 (8:1), and KC3-C15 (C8:1). In some embodiments, the KC3 ionizable cationic lipid is KC3-OA. In some embodiments, the KC3 ionizable cationic lipid is KC3-PA. In some embodiments, the KC3 ionizable cationic lipid is KC3-C17(C8:1). In some embodiments, the KC3 ionizable cationic lipid is KC3-C15(C8:1). In some embodiments, the conjugated lipid is PEG-DMG or PEG-DSG. In some embodiments, the composition comprises the PEG-containing conjugated lipid in a total amount of 0.5-2.0 mol % of the total lipid content of the LNP composition. In some embodiments, the composition comprises the ionizable cationic lipid in a total amount of 48 mol % of the total lipid content of the LNP composition. In some embodiments, the composition comprises DSPC and DSPS in a total amount of 10 mol % of the total lipid content of the LNP composition. In some embodiments, the composition comprises 5% DSPC or HSPC in a total amount of 5 mol % of the total lipid content of the LNP composition. In some embodiments, the composition comprises PEG-DMG in a total of 1.5 mol % of the total lipid content of the LNP composition. In some embodiments, the composition comprises cholesterol in a total amount of 40.5 mol % cholesterol of the total lipid content of the LNP composition. In some embodiments, the composition comprises the DSPC phospholipid in a total amount of 10 mol % of the total lipid content of the LNP composition. In some embodiments, the PEG-containing conjugated lipid is PEG₂₀₀₀-DMG.

Aspects of the disclosure relate to a nucleic acid lipid nanoparticle (LNP) composition comprising: a nucleic acid; an ionizable cationic lipid in a total amount of 40-65 mol % of the total lipid content of the LNP composition; a sterol in a total amount of 25-45 mol % of the total lipid content of the LNP composition; and one or more phospholipids in a total amount of phospholipids of 5-25 mol % of the total lipid content of the LNP composition, and comprising a phosphatidylserine (PS) in a total amount of 2.5-10 mol % of the total lipid content of the LNP composition; and optionally a conjugated lipid in a total amount of 0.5-2.5 mol % of the total lipid content of the LNP composition. In some embodiments, the one or more phospholipids consist of: DSPC and a L-serine PS. In some embodiments, the composition comprises the PS in a total amount of 2.5-7.5 mol % of the total lipid in the composition. In some embodiments, the conjugated lipid comprises PEG. In some embodiments, conjugated lipid is PEG-DMG.

In some embodiments, the disclosure provides certain LNP compositions. In some aspects, the LNP compositions comprise: a nucleic acid; an ionizable cationic lipid at a N/P ratio of 3 to 8 relative to the nucleic acid in a total amount of 40-65 mol % of the total lipid content of the LNP composition; a sterol in a total amount of 0.5-50 mol % of the total lipid content of the LNP composition; one or more phospholipids in a total amount of phospholipids of 5-50 mol % of the total lipid content of the LNP composition; and a conjugated lipid in a total amount of 0.5-2.5 mol % of the total lipid content of the LNP composition. In some aspects, the LNP composition is further characterized in that: the nucleic acid is mRNA; the ionizable cationic lipid is present in the LNP composition at a N/P ratio of 4 to 7 relative to the nucleic acid; the sterol is cholesterol; and the conjugated lipid is a PEG-containing conjugated lipid. In some aspects, the one or more phospholipids in the LNP comprise at least two phospholipids having mismatched acyl chain lengths. In some aspects, the one or more phospholipids in the LNP comprise a phosphatidylserine (PS) lipid in a total amount of 2.5-10 mol % of the total lipid content of the LNP composition. In some aspects, the phosphatidylserine (PS) lipid in the LNP consists of, consists essentially of or comprises dipalmitoylphosphatidyl-L-serine ((L-serine)DPPS). In some aspects, the one or more phospholipids in the LNP comprise a phospholipid selected from the group consisting of: distearoylphosphatidylcholine (DSPC) and hydrogenated soy phosphatidylcholine (HSPC). In some aspects, the one or more phospholipids in the LNP consist of distearoylphosphatidylcholine (DSPC) and dipalmitoylphosphatidyl-L-serine ((L-serine)DPPS). In some aspects, the PEG-containing conjugated lipid in the LNP is PEG(2000)-dimyristoylglycerol (PEG-DMG).

In some embodiments, a lipid nanoparticle (LNP) vaccine composition comprises: a nucleic acid; a KC3 ionizable cationic lipid at a N/P ratio of 3 to 7 relative to the nucleic acid in a total amount of 46-54 mol % of the total lipid content of the LNP composition; one or more phospholipids in a total amount of phospholipids of 5-20 mol % of the total lipid content of the LNP composition; a conjugated lipid in a total amount of 1.0-3.5 mol % of the total lipid content of the LNP composition; and cholesterol. In some embodiments, the one or more phospholipids in the LNP comprises an anionic phospholipid in a total of 2-8 mol % of the total lipid content of the LNP composition. In some aspects, the anionic phospholipid is a phosphatidylserine (PS). In some embodiments, the anionic phospholipid is an anionic phospholipid selected from the group consisting of: distearoylphosphatidylglycerol (DSPG) and dipalmitoyphosphatidylglycerol (DPPG). In some embodiments, the anionic phospholipid is an anionic phospholipid selected from the group consisting of: dipalmitoylphosphatidyl-L-serine (DPPS), or distearoylphosphatidyl-L-serine (DSPS).

In some aspects, the LNP composition has 5-50 mol % total phospholipid, including compositions with 5, 10, 15, 20, 25, 30, 35, 40, 45 or 50 mol % total phospholipid. In some embodiments, the LNP composition is further characterized by: the sterol in a total amount of 0.5-45.5 mol % of the total lipid content of the LNP composition; and the one or more phospholipids in a total amount of phospholipids of 5-50 mol % of the total lipid content of the LNP composition. In some embodiments, the sterol in the LNP composition is cholesterol. In some embodiments, the ionizable cationic lipid is KC3-OA (Racemic). In some embodiments, the ionizable cationic lipid is KC3-OA(S). In some embodiments, the ionizable cationic lipid is KC3-OA(R). In some embodiments, the ionizable cationic lipid is KC4-OA (Racemic). In some embodiments, the ionizable cationic lipid is KC4-OA(S). In some embodiments, the ionizable cationic lipid is KC3-OA(R). In some embodiments, the ionizable cationic lipid is a mixture of KC3-OA and KC4-OA. In some embodiments, the LNP composition comprises a total of 48-54 mol % of the ionizable cationic lipid.

In some embodiments, the LNP comprises at least two types of lipids. In an embodiment, the LNP comprises two of an ionizable lipid, a phospholipid, a sterol, and an alkylene glycol-containing lipid. In some embodiments, the LNP comprises at least three types of lipids. In an embodiment, the LNP comprises three of an ionizable lipid, a phospholipid, a sterol, and an alkylene glycol-containing lipid. In some embodiments, the LNP comprises at least four types of lipids. In an embodiment, the LNP comprises each of an ionizable lipid, a phospholipid, a sterol, and an alkylene glycol-containing lipid.

The LNP (e.g., as described herein) may comprise one or more of the following components: (i) an ionizable cationic lipid at a concentration between about 1 mol % to about 95 mol % (e.g. about 20 mol % to about 80 mol %); (ii) a phospholipid at a concentration between 0.1 mol % to about 50 mol % (e.g. between about 2.5 mol % to about 20 mol %); (iii) a sterol at a concentration between about 1 mol % to about 95 mol % (e.g. about 20 mol % to about 80 mol %); and (iv) a PEG-containing lipid at a concentration between about 0.1 mol % to about 50 mol % (e.g. between about 2.5 mol % to about 20 mol %). In an embodiment, the LNP comprises one of (i)-(iv). In an embodiment, the LNP comprises two of (i)-(iv). In an embodiment, the LNP comprises three of (i)-(iv). In an embodiment, the LNP comprises each of (i)-(iv). In some embodiments, the LNP comprises (i) and (ii). In some embodiments, the LNP comprises (i) and (iii). In some embodiments, the LNP comprises (i) and (iv). In some embodiments, the LNP comprises (ii) and (iii). In some embodiments, the LNP comprises (ii) and (iv). In some embodiments, the LNP comprises (iii) and (iv). In some embodiments, the LNP comprises (i), (ii), and (iii). In some embodiments, the LNP comprises (i), (ii), and (iv). In some embodiments, the LNP comprises (ii), (iii), and (iv).

The LNP (e.g., as described herein) may comprise one or more of the following components: (i) Ionizable cationic lipid (ICL) at a concentration between about 1 mol % to about 95 mol % (e.g. about 20 mol % to about 80 mol %); (ii) DSPC at a concentration between 0.1 mol % to about 50 mol % (e.g. between about 2.5 mol % to about 20 mol %); (iii) cholesterol at a concentration between about 1 mol % to about 95 mol % (e.g. about 20 mol % to about 80 mol %); and (iv) DMG-PEG2k at a concentration between about 0.1 mol % to about 50 mol % (e.g. between about 2.5 mol % to about 20 mol %). In an embodiment, the LNP comprises two of (i)-(iv). In an embodiment, the LNP comprises three of (i)-(iv). In an embodiment, the LNP comprises each of (i)-(iv). In some embodiments, the LNP comprises (i) and (ii). In some embodiments, the LNP comprises (i) and (iii). In some embodiments, the LNP comprises (i) and (iv). In some embodiments, the LNP comprises (ii) and (iii). In some embodiments, the LNP comprises (ii) and (iv). In some embodiments, the LNP comprises (iii) and (iv). In some embodiments, the LNP comprises (iii) and (iv). In some embodiments, the LNP comprises (i), (ii), and (iii). In some embodiments, the LNP comprises (i), (ii), and (iv). In some embodiments, the LNP comprises (ii), (iii), and (iv).

In some embodiments, the ionizable cationic lipid in a total amount of 45-55 mol % of the total lipid content of the LNP composition; cholesterol is in a total amount of 35-45 mol % of the total lipid content of the LNP composition; the total amount of the one more phospholipid is 7-15 mol % of the total lipid content of the LNP composition; the one or more phospholipids consist of DSPC and the PS lipid is one or more lipids selected from the group consisting of the L-serine configuration of DPPS and DSPS; and the total amount of the PS lipid is about 5 mol % of the total lipid content of the LNP composition.

In an embodiment, the LNP comprises a ratio of ionizable lipid to phospholipid of about 50:1 to about 1:1 (e.g., 40:1, 32:3, 6:1, 7:1, 5:1, 24:5, 26:5, 10:3, 15:2, 16:7, 18:1, 3:1, 3:2, or 1:1). In an embodiment, the LNP comprises a ratio of ionizable lipid to phospholipid of about 15:2. In an embodiment, the LNP comprises a ratio of ionizable lipid to phospholipid of about 5:1. In an embodiment, the LNP comprises a ratio of ionizable lipid to a sterol of about 10:1 to about 1:10 (e.g., 9:1, 8:1, 8:7, 7:1, 7:5, 7:3, 6:1, 6:5, 5:1, 5:3, 4:1, 4:3, 3:1, 2:1, 1:1, 1:2, 1:3, 3:4, 1:4, 3:5, 1:5, 4:5, 1:6, 5:6, 7:6, 7:8, or 8:9). In an embodiment, the LNP comprises a ratio of ionizable lipid to an alkylene-containing lipid of about 1:10 to about 10:1 (e.g., 1:9, 1:8, 7:8, 7:1, 7:5, 7:3, 6:1, 6:5, 5:1, 5:3, 4:1, 4:3, 3:1, 2:1, 1:1, 1:2, 1:3, 3:4, 1:4, 3:5, 1:5, 4:5, 1:6, 5:6, 7:6, 7:8, or 8:9). In an embodiment, the LNP comprises a ratio of phospholipid to an alkylene-containing lipid of about 10:1 to about 1:10 (e.g., 9:1, 8:1, 8:7, 7:1, 7:5, 7:3, 6:1, 6:5, 5:1, 5:3, 4:1, 4:3, 3:1, 2:1, 1:1, 1:2, 1:3, 3:4, 1:4, 3:5, 1:5, 4:5, 1:6, 5:6, 7:6, 7:8, or 8:9). In an embodiment, the LNP comprises a ratio of a sterol to an alkylene-containing lipid of about 50:1 to about 1:1 (e.g., 40:1, 32:3, 6:1, 7:1, 5:1, 24:1, 22:1, 20:1, 22:5, 24:5, 26:5, 10:3, 15:2, 16:7, 18:1, 3:1, 3:2, or 1:1).

In an embodiment, a LNP (e.g., described herein) comprises two of an ionizable lipid, a phospholipid, a sterol, and an alkylene glycol-containing lipid (e.g., PEG-containing lipid). In another embodiment, a LNP (e.g., described herein) comprises three of an ionizable lipid, a phospholipid, a sterol, and an alkylene glycol-containing lipid (e.g., PEG-containing lipid). In an embodiment LNP (e.g., described herein) comprises each of an ionizable lipid, a phospholipid, a sterol, and an alkylene glycol-containing lipid (e.g., PEG-containing lipid).

The present disclosure features a lipid nanoparticle comprising mRNA and lipids. Exemplary lipids include ionizable cationic lipids (ICLs), phospholipids, sterol lipids, alkylene glycol lipids (e.g., polyethylene glycol lipids), sphingolipids, glycerolipids, glycerophospholipids, prenol lipids, saccharolipids, fatty acids, and polyketides. In some embodiments, the LNP comprises a single type of lipid. In some embodiments, the LNP comprises a plurality (e.g. two or more) of lipids. An LNP may comprise one or more of an ionizable cationic lipid, a phospholipid, a sterol, or an alkylene glycol lipid (e.g., a polyethylene glycol lipid).

In some embodiments, the lipidic nanoparticle comprises a membrane comprising phosphatidylcholine and a sterol. In some embodiments, the sterol is cholesterol. In some embodiments, the lipidic nanoparticle comprises a membrane comprising phosphatidylcholine, ionizable cationic lipid (ICL).

The LNP (e.g., as described herein) may comprise one or more of the following components: (i) Ionizable cationic lipid (ICL) containing a C16 alkyl or C16 alkenyl group or C18 alkyl or C18 alkenyl group at a concentration between about 1 mol % to about 95 mol % (or any value therebetween, e.g. about 20 mol % to about 80 mol %); (ii) A phospholipid at a concentration between 0.1 mol % to about 20 mol % (or any value there between, e.g. between about 2.5 mol % to about 10 mol %) where the phospholipid also contains C16 or C18 alkyl or alkenyl groups; (iii) cholesterol at a concentration between about 1 mol % to about 95 mol % (or any value therebetween, e.g. about 20 mol % to about 80 mol %); (iv) a phosphatidylserine (PS) or phosphatidylglycerol (PG) added to the LNP lipid formulation at a concentration between about 0.5 mol % to about 20 mol %, about 2.5 mol % to about 10 mol %, about 4 mol % to about 8 mol %, or any value therebetween of the total lipid content of the LNP, and (v) a polyethyleneglycol (PEG)-2000-containing lipid (e.g., DPG-PEG2000, DPPE-PEG2000, DMPE-PEG2000, DMG-PEG2000) at a concentration between about 0.1 mol % to about 5 mol % (or any value therebetween, e.g. between about 1 mol % to about 2.5 mol %). In an embodiment, the LNP comprises two of (i)-(v). In an embodiment, the LNP comprises three of (i)-(v). In an embodiment, the LNP comprises four of (i)-(v). In an embodiment, the LNP comprises each of (i)-(v). In some embodiments, the LNP comprises (i) and (ii). In some embodiments, the LNP comprises (i) and (iii). In some embodiments, the LNP comprises (i) and (v). In some embodiments, the LNP comprises (ii) and (iii). In some embodiments, the LNP comprises (ii) and (v). In some embodiments, the LNP comprises (iii) and (iv). In some embodiments, the LNP comprises (iii) and (v). In some embodiments, the LNP comprises (i), (ii), and (iii). In some embodiments, the LNP comprises (i), (ii), and (v). In some embodiments, the LNP comprises (ii), (iii), and (v). In some embodiments, the LNP comprises (ii), (iii), (iv) and (v). In an embodiment, the LNP consists or consists essentially of four of (i)-(v). In an embodiment, the LNP consists or consists essentially of each of (i)-(v). In some embodiments, the LNP consists or consists essentially of (i) and (ii). In some embodiments, the LNP consists or consists essentially of (i) and (iii). In some embodiments, the LNP consists or consists essentially of (i) and (v). In some embodiments, the LNP consists or consists essentially of (ii) and (iii). In some embodiments, the LNP comprises (ii) and (v). In some embodiments, the LNP consists or consists essentially of (iii) and (iv). In some embodiments, the LNP consists or consists essentially of (iii) and (v). In some embodiments, the LNP consists or consists essentially of (i), (ii), and (iii). In some embodiments, the LNP consists or consists essentially of (i), (ii), and (v). In some embodiments, the LNP comprises (ii), (iii), and (v). In some embodiments, the LNP consists or consists essentially of (ii), (iii), (iv) and (v).

In some embodiments, the ICL have a structure of Formula I, II, III, IV-B, V-A-1, and cholesterol, wherein the membrane separates the inside of the lipidic nanoparticles from the aqueous medium. In some embodiment, the ICL have a structure as shown in Table 1. In some embodiments, the phosphatidylcholine is distearoylphosphatidylcholine (DSPC) or hydrogenated soy phosphatidylcholine (HSPC). In some embodiments, the ionizable cationic lipid to cholesterol molar ratios is from about 65:35 to 40:60. In some embodiments, the ICL to cholesterol molar ratio is from about 60:40 to about 45:55.

In some embodiments, the phosphatidylcholine to cholesterol molar ratio is from about 1:5 to about 1:2.

In some embodiments, the membrane further comprises a polymer-conjugated lipid.

In some embodiments, the lipidic nanoparticle comprises ICL, DSPC, cholesterol and polymer-conjugated lipid in a about 49.5:10.3:39.6:2.5 molar ratio.

Aspects of the disclosure relate to a nucleic acid lipid nanoparticle (LNP) composition comprising: a nucleic acid, ionizable cationic lipid KC3-PA, and a (L-Serine) PS lipid in a total amount of 2.5-10 mol % of the total lipid content of the LNP composition.

In some embodiments, the nucleic acid is mRNA, the PS lipid is (L-Serine) DSPS, (L-Serine) DPPS, or a mixture thereof, and the LNP composition further comprises cholesterol and a second phospholipid selected from the group consisting of: DSPC, DOPC, DPPC, HSPC, and SM.

Aspects of the disclosure relate to a nucleic acid lipid nanoparticle (LNP) composition comprising: a nucleic acid, an ionizable cationic lipid selected from KC3-C17 (C8:1); and a (L-Serine) PS lipid in a total amount of 2.5-10 mol % of the total lipid content of the LNP composition.

In some embodiments, the phospholipids are DSPC and DPPS. In some embodiments, the DSPC and DPPS are each present in the LNP at a total amount of 5 mol % each, based on the total lipid content of the LNP composition.

Aspects of the disclosure relate to a nucleic acid lipid nanoparticle (LNP) composition comprising: a nucleic acid, ionizable cationic lipid KC3-PA or KC3-OA, and a (L-Serine) PS lipid in a total amount of 2.5-10 mol % of the total lipid content of the LNP composition.

In some embodiments, the nucleic acid is mRNA, the PS lipid is (L-Serine) DSPS, (L-Serine) DPPS, or a mixture thereof, and the LNP composition further comprises cholesterol and a second phospholipid selected from the group consisting of: DSPC, DPPC, HSPC, and SM.

In some embodiments, the LNP composition further comprises 0.5-2.0 mol % PEG-DMG or PEG-DSG, based on the total lipid content in the LNP composition.

In some embodiments, the ionizable cationic lipid is KC3-PA. In some embodiments, the ionizable cationic lipid KC3-OA.

Aspects of the disclosure relate to a nucleic acid lipid nanoparticle (LNP) composition comprising: a nucleic acid, a KC3-C17 (C8:1) ionizable cationic lipid; and a (L-Serine) PS lipid in a total amount of 2.5-10 mol % of the total lipid content of the LNP composition.

Aspects of the disclosure relate to a nucleic acid lipid nanoparticle (LNP) composition comprising: a nucleic acid; an ionizable cationic lipid, the ionizable cationic lipid in a total amount of 45-55 mol % of the total lipid content of the LNP composition; a sterol, wherein the sterol is cholesterol in a total amount of 35-45 mol % of the total lipid content of the LNP composition; one or more phospholipids, wherein the one or more phospholipids in a total amount of phospholipids of 10 mol % of the total lipid content of the LNP composition, and comprising a phosphatidylserine (PS) in a total amount of 3-9 mol % of the total lipid content of the LNP composition; and a conjugated lipid, the conjugated lipid in a total amount of 0.5-2.0 mol % of the total lipid content of the LNP composition.

In some embodiments, the one or more phospholipid is selected from the group consisting of: DSPS (L-isomer), DPPS (L-isomer), DMPS (L-isomer), DOPS (L-isomer), and DSPS (D-isomer).

In some embodiments, the conjugated lipid is PEG-DMG; and the PS lipid is selected from the group consisting of: DSPS (L-isomer) and DPPS.

In some embodiments, the ionizable cationic lipid is one or more compounds selected from the group consisting of: KC3-OA, KC3-PA, KC3-C17 (8:1), KC3-C15 (C8:1), AKG-OA-DM2, AKG-OA-DM3, DODAP, and DODMA. In some embodiments, the ionizable cationic lipid is AKG-OA-DM2. In some embodiments, the ionizable cationic lipid is KC3-OA. In some embodiments, the ionizable cationic lipid is DODMA.

In some embodiments, the LNP comprises a nucleic acid; an ionizable cationic lipid in a total amount of 50 mol % of the total lipid content of the LNP composition; cholesterol in a total amount of 38.5 mol % of the total lipid content of the LNP composition; one or more phospholipids in a total amount of 7-15 mol % of the total lipid content of the LNP composition, and comprising a phosphatidylserine (PS) lipid in a total amount of 3-9 mol % of the total lipid content of the LNP composition; and a PEG-containing lipid in a total amount of 0.5-2.0 mol % of the total lipid content of the LNP composition.

In some embodiments, the comprises a nucleic acid; the ionizable lipid described herein, a sterol; one or more phospholipids comprising a phosphatidylserine (PS) lipid; and optionally a conjugated lipid. In some embodiments, the nucleic acid is mRNA. In some embodiments, the sterol is cholesterol. In some embodiments, the one or more phospholipids consist of: one or more phospholipids selected from the group consisting of: SM, DSPC, HSPC, DPPC and DOPC; and a PS lipid selected from the group consisting of: DPPS, and DSPS. In some embodiments, the one or more phospholipids consist of: DSPC; and one or more PS lipids selected from the group consisting of (L-Serine) DPPS and (L-Serine) DSPS. In some embodiments, the composition comprises the PS lipid in a total amount of 2.5-10 mol % of the total lipid in the composition. In some embodiments, the conjugated lipid comprises PEG.

In some embodiments, the conjugated lipid is PEG-DMG; and the PS lipid is selected from the group consisting of: DSPS (L-isomer) and DPPS.

In some embodiments, lipid nanoparticle (LNP) compositions comprising an ionizable cationic lipid compositions are provided. In some embodiments, lipid nanoparticle (LNP) compositions comprising an ionizable cationic lipid are provided. In some embodiments, the LNP composition comprises a TLR-activating nucleic acid. In some embodiments, a lipid nanoparticle (LNP) composition further comprises the PS lipid in a total amount of 2.5-10 mol % of the total lipid in the composition of the LNP. In some embodiments, a lipid nanoparticle (LNP) composition further comprises a PS lipid selected from the group consisting of: DSPS (L-isomer) and DPPS. In some embodiments, a lipid nanoparticle (LNP) composition comprises a conjugated lipid in a total amount of 0.5-2.0 mol % of the total lipid content of the LNP composition. In some embodiments, a lipid nanoparticle (LNP) composition comprises the conjugated lipid in a total amount of less than 2 mol % of the total lipid content of the LNP composition, and the conjugated lipid is PEG-DMG.

In some embodiments, a lipid nanoparticle (LNP) composition comprises a nucleic acid; an ionizable lipid disclosed herein; a sterol; one or more phospholipids comprising a phosphatidylserine (PS) lipid; and optionally further comprising a conjugated lipid. In some embodiments, a lipid nanoparticle (LNP) composition comprises a TLR-activating nucleic acid; an ionizable lipid disclosed herein; cholesterol; one or more phospholipids selected from the group consisting of: SM, DSPC, HSPC, DPPC and DOPC; and a PS lipid selected from the group consisting of: DPPS and DSPS; and optionally further comprising a conjugated lipid comprising PEG. In some embodiments, a nucleic acid lipid nanoparticle (LNP) composition comprises: a nucleic acid; an ionizable cationic lipid in a total amount of 40-65 mol % of the total lipid content of the LNP composition; a sterol in a total amount of 25-45 mol % of the total lipid content of the LNP composition; and one or more phospholipids in a total amount of phospholipids of 5-25 mol % of the total lipid content of the LNP composition, and comprising a phosphatidylserine (PS) in a total amount of 2.5-10 mol % of the total lipid content of the LNP composition; and optionally further comprising a conjugated lipid in a total amount of 0.5-2.5 mol % of the total lipid content of the LNP composition.

In some embodiments, the LNP composition further comprises an anionic lipid selected from the group consisting of: DSPS (L-isomer), DPPS (L-isomer), DMPS (L-isomer), DOPS (L-isomer), and DSPS (D-isomer).

In some aspects, the LNP composition consists of: 48 mol % KC3-OA; 5 mol % DPPS or DSPG; 5-10 mol % DSPC or HSPC; 1.5 mol % PEG-DMG; and cholesterol. In some aspects, the LNP composition consists of: 48 mol % KC3-OA; 5 mol % DPPS or DSPG; 5 mol % DSPC or HSPC; 1.5 mol % PEG-DMG; and 40.5 mol % cholesterol. In some aspects, the LNP composition consists of: 48 mol % KC3-OA; 5 mol % DPPS or DSPG; 10 mol % DSPC or HSPC; 1.5 mol % PEG-DMG; and 35.5 mol % cholesterol. In some aspects, the LNP composition consists of: 48 mol % ionizable cationic lipid; 5 mol % DPPS or DSPG; 5-10 mol % DSPC or HSPC; 1.5 mol % PEG-DMG; and cholesterol. In some aspects, the LNP composition consists of: 48 mol % ionizable cationic lipid; 5 mol % DPPS or DSPG; 5 mol % DSPC or HSPC; 1.5 mol % PEG-DMG; and 40.5 mol % cholesterol. In some aspects, the LNP composition consists of: 48 mol % ionizable cationic lipid; 5 mol % DPPS or DSPG; 10 mol % DSPC or HSPC; 1.5 mol % PEG-DMG; and 35.5 mol % cholesterol.

In some embodiments, the composition comprises 48 mol % of the ionizable cationic lipid, 40.5 mol % cholesterol, 5 mol % (L-Serine) DPPS lipid, 5 mol % DSPC or DPPC; and a total of 10 mol % phospholipid concentration, wherein each mol % refers to the mol % of the total lipid content of the LNP composition.

Aspects of the disclosure relate to the use of a (L-Serine) PS lipid in combination with an ionizable cationic lipid described herein in the LNP for targeting of the LNP to dendritic cells. In some embodiments, the LNP comprises TLR-activating RNA or DNA. In some embodiments, the LNP further comprises cholesterol. In some embodiments, the total amount of (L-Serine) PS lipid in the LNP is 2.5-10 mol % of the total lipid content of the LNP composition. In some embodiments, the LNP further comprises one or more additional phospholipids including DSPC. In some embodiments, the LNP further comprises a conjugated lipid. In some embodiments, the LNP comprises: a TLR-activating nucleic acid with a N/P ratio of 3 to 8; a AKG-OA-DM2 or DODMA ionizable cationic lipid (ICL), in a total amount of 40-65 mol % of the total lipid content of the LNP composition; cholesterol in a total amount of 25-40 mol % of the total lipid content of the LNP composition; a (L-Serine) PS lipid in a total amount of 2.5-10 mol % of the total lipid content of the LNP composition; DSPC phospholipid in a total amount of 5-25 mol % of the total lipid content of the LNP composition; and a conjugated lipid in a total amount of 0-2.5 mol % of the total lipid content of the LNP composition. In some embodiments, the ICL is AKG-OA-DM2. In some embodiments, the ICL is DODMA.

Aspects of the disclosure relate to a nucleic acid lipid nanoparticle (LNP) vaccine composition comprising: a nucleic acid with a N/P ratio of 4 to 7; an KC3-PA ionizable cationic lipid in a total amount of 40-65 mol % of the total lipid content of the LNP composition; cholesterol in a total amount of 25-40 mol % of the total lipid content of the LNP composition; a (L-Serine) PS lipid in a total amount of 2.5-10 mol % of the total lipid content of the LNP composition; DSPC phospholipid in a total amount of 5-25 mol % of the total lipid content of the LNP composition; and PEG-DMG in a total amount of 0-2.5 mol % of the total lipid content of the LNP composition.

Aspects of the disclosure relate to a nucleic acid lipid nanoparticle (LNP) vaccine composition comprising: a TLR-activating nucleic acid with a N/P ratio of 3 to 8; a KC3-C17 (C8:1) ionizable cationic lipid in a total amount of 40-65 mol % of the total lipid content of the LNP composition; cholesterol in a total amount of 25-40 mol % of the total lipid content of the LNP composition; a (L-Serine) PS lipid in a total amount of 2.5-10 mol % of the total lipid content of the LNP composition; DSPC phospholipid in a total amount of 5-25 mol % of the total lipid content of the LNP composition; and PEG-DMG in a total amount of 0-2.5 mol % of the total lipid content of the LNP composition.

Aspects of the disclosure relate to a nucleic acid lipid nanoparticle (LNP) vaccine composition comprising: a nucleic acid with a N/P ratio of 4 to 7; a KC3-OA-DM2 ionizable cationic lipid in a total amount of 40-65 mol % of the total lipid content of the LNP composition; cholesterol in a total amount of 25-40 mol % of the total lipid content of the LNP composition; a (L-Serine) PS lipid in a total amount of 2.5-10 mol % of the total lipid content of the LNP composition; DSPC phospholipid in a total amount of 5-25 mol % of the total lipid content of the LNP composition; and PEG-DMG in a total amount of 0-2.5 mol % of the total lipid content of the LNP composition.

Aspects of the disclosure relate to a nucleic acid lipid nanoparticle (LNP) vaccine composition comprising: a nucleic acid with a N/P ratio of 3 to 8; a DODMA ionizable cationic lipid in a total amount of 40-65 mol % of the total lipid content of the LNP composition; cholesterol in a total amount of 25-40 mol % of the total lipid content of the LNP composition; a (L-Serine) PS lipid in a total amount of 2.5-10 mol % of the total lipid content of the LNP composition; DSPC phospholipid in a total amount of 5-25 mol % of the total lipid content of the LNP composition; and PEG-DMG in a total amount of 0-2.5 mol % of the total lipid content of the LNP composition.

In some embodiments, the nucleic acid is a DNA comprises a TLR9-activating DNA oligonucleotide sequence. In some embodiments, the RNA comprises a TLR7 and/or TLR8-activating RNA oligonucleotide sequence. In some embodiments, nucleic acid includes both a TLR-activating DNA oligonucleotide and a TLR-activating RNA oligonucleotide.

In some embodiments, the LNP is a TLR-activating nucleic acid lipid nanoparticle vaccine composition comprising: a TLR-activating nucleic acid with a N/P ratio of 4 to 7; an ionizable cationic lipid in a total amount of 40-65 mol % of the total lipid content of the LNP composition; cholesterol in a total amount of 25-40 mol % of the total lipid content of the LNP composition; a (L-Serine) PS lipid in a total amount of 2.5-10 mol % of the total lipid content of the LNP composition; DSPC phospholipid in a total amount of 5-25 mol % of the total lipid content of the LNP composition; and PEG-DMG in a total amount of 0-2.5 mol % of the total lipid content of the LNP composition.

In some embodiments, the LNP composition comprises the ionizable cationic lipid in a total amount of 46-65 mol % of the total lipid content of the LNP composition. In some embodiments, the LNP composition comprises the PS in a total amount of about 5 mol % of the total lipid in the composition. In some embodiments, the LNP composition comprises the conjugated lipid in a total amount of about 1.5 mol % of the total lipid content of the LNP composition. In some embodiments, the conjugated lipid is PEG-DMG; and the PS lipid is selected from the group consisting of: DSPS (L-isomer) and DPPS. In some embodiments, the ionizable cationic lipid is one or more compounds selected from the group consisting of: AKG-OA-DM2, DODMA, KC3-OA, KC3-PA, KC3-C17 (C8:1), and KC3-C15 (C8:1). In some embodiments, the ionizable cationic lipid is KC3-PA. In some embodiments, the ionizable cationic lipid is KC3-OA. In some embodiments, the ionizable cationic lipid is AKG-OA-DM2.

In some embodiments, the LNP comprises mRNA. In some embodiments, the LNP further comprises cholesterol. In some embodiments, the total amount of (L-Serine) PS lipid in the LNP is 2.5-10 mol % of the total lipid content of the LNP composition. In some embodiments, the LNP further comprises one or more additional phospholipids including DSPC. In some embodiments, the LNP further comprises a conjugated lipid.

In some embodiments, the LNP comprises: a TLR-activating nucleic acid with a N/P ratio of 3 to 8; a KC3-PA or KC3-C17 (C8:1) ionizable cationic lipid (ICL), in a total amount of 40-65 mol % of the total lipid content of the LNP composition; cholesterol in a total amount of 25-40 mol % of the total lipid content of the LNP composition; a (L-Serine) PS lipid in a total amount of 2.5-10 mol % of the total lipid content of the LNP composition; DSPC phospholipid in a total amount of 5-25 mol % of the total lipid content of the LNP composition; and a conjugated lipid in a total amount of 0-2.5 mol % of the total lipid content of the LNP composition.

In some embodiments, the ICL is KC3-PA. In some embodiments, the ICL is KC3-C17 (C8:1).

In some embodiments, the composition comprises an anionic phospholipid selected from the group consisting of: DOPG, DSPG, and DPPG, in a total amount of 2.5-7.5% of the total lipid content of the LNP composition. In some embodiments, the composition comprises DSPG anionic phospholipid in a total amount of 2.5-7.5% of the total lipid content of the LNP composition. In some embodiments, the composition comprises DPPG anionic phospholipid in a total amount of 2.5-7.5% of the total lipid content of the LNP composition.

In some embodiments, the LNP further comprises one or more additional phospholipids including DSPC.

Aspects of the disclosure relate to a TLR-activating nucleic acid lipid nanoparticle (LNP) composition comprising: a TLR-activating nucleic acid; a KC3 ionizable cationic lipid selected from the group consisting of: KC3-OA, KC3-PA, KC3-C17 (8:1), and KC3-C15 (C8:1), in a total amount of 45-55 mol % of the total lipid content of the LNP composition; cholesterol in a total amount of 33.5-43.5 mol % of the total lipid content of the LNP composition; a (L-Serine) DPPS lipid in a total amount of 5 mol % of the total lipid content of the LNP composition; DSPC or HSPC phospholipid in a total amount of 5 mol % of the total lipid content of the LNP composition; and a PEG-DMG conjugated lipid in a total amount of 1.5 mol % of the total lipid content of the LNP composition.

Aspects of the disclosure relate to a lipid nanoparticle (LNP) composition comprising a KC3 ionizable cationic lipid, a (L-Serine) PS lipid, cholesterol, one or more phospholipids comprising at least one anionic phospholipid, and a conjugated lipid, wherein the LNP is obtained by a process comprising the step of dissolving a sodium or ammonium salt of the anionic phospholipid.

In some embodiments, the composition comprises a TLR-activating nucleic acid. In some embodiments, the TLR-activating nucleic acid is an RNA oligonucleotide.

In some embodiments, the total amount of phospholipids in the composition is 5-25 mol % of the total lipid content of the LNP composition, and the total amount of the phosphatidylserine (PS) is 2.5-10 mol % of the total lipid content of the LNP composition; and the total amount of the conjugated lipid in the composition is a total amount of 0.5-2.5 mol % of the total lipid content of the LNP composition.

In some embodiments, the composition comprises 48 mol % of the KC3 ionizable cationic lipid, 40.5 mol % cholesterol, and 5 mol % (L-Serine) DPPS lipid, wherein each mol % refers to the mol % of the total lipid content of the LNP composition.

In some embodiments, the composition comprises 48 mol % of the KC3 ionizable cationic lipid, 38.5 mol % cholesterol, and 5 mol % (L-Serine) DPPS lipid, wherein each mol % refers to the mol % of the total lipid content of the LNP composition.

In some embodiments, the composition comprises 46-54 mol % of the KC3 ionizable cationic lipid, and 5 mol % (L-Serine) DPPS lipid, wherein each mol % refers to the mol % of the total lipid content of the LNP composition.

In some embodiments, the composition comprises 45 mol % of the KC3 ionizable cationic lipid, 42.7 mol % cholesterol, and 5 mol % (L-Serine) DPPS lipid, wherein each mol % refers to the mol % of the total lipid content of the LNP composition.

In some embodiments, the composition comprises 50 mol % of the KC3 ionizable cationic lipid, 38.5 mol % cholesterol, 5 mol % (L-Serine) DPPS lipid, and a total of 10 mol % phospholipid concentration; wherein each mol % refers to the mol % of the total lipid content of the LNP composition.

In some embodiments, the composition comprises 48 mol % of the KC3 ionizable cationic lipid, 40.5 mol % cholesterol, 5 mol % (L-Serine) DPPS lipid, and a total of 10 mol % phospholipid concentration; wherein each mol % refers to the mol % of the total lipid content of the LNP composition.

In some embodiments, the composition comprises 48 mol % of the KC3 ionizable cationic lipid, 40.5 mol % cholesterol, 5 mol % (L-Serine) DPPS lipid, 5 mol % DSPC or DPPC; and a total of 10 mol % phospholipid concentration; wherein each mol % refers to the mol % of the total lipid content of the LNP composition.

In some embodiments, the composition comprises 46.5 mol % of the KC3 ionizable cationic lipid, 42 mol % cholesterol, 5 mol % (L-Serine) DPPS lipid, wherein each mol % refers to the mol % of the total lipid content of the LNP composition.

In some embodiments, the composition further comprises a total of 5 mol % DSPC or HSPC of the total lipid content of the LNP composition.

In some embodiments, the composition further comprises a total of 1.5 mol % PEG-DMG of the total lipid content of the LNP composition.

In some embodiments, the composition comprises a total of 10 mol % of DSPC/DPPC phospholipid of the total lipid content of the LNP composition.

Aspects of the disclosure relate to a phosphatidylserine salt selected from the group consisting of DSPS sodium, DPPS sodium, DSPS ammonium and DPPS ammonium.

Aspects of the disclosure relate to the use of a DSPS-Na salt or a DPPS-NH₄ salt in the preparation of a LNP comprising a (L-Serine) PS lipid, a sterol, a conjugated lipid, a phospholipid for targeting the LNP to dendritic cells.

Aspects of the disclosure relate to a solution comprising ethanol and DSPS or DPPS, the solution obtained by a process comprising the step of dissolving a phosphatidylserine salt in ethanol, wherein the phosphatidylserine salt is selected from the group consisting of DSPS sodium, DPPS sodium, DOPS sodium, DOPS ammonium, DSPS ammonium and DPPS ammonium.

In some embodiments, the TLR-activating nucleic acid is a DNA oligonucleotide TLR 9 agonist, the ionizable cationic lipid in a total amount of 45-55 mol % of the total lipid content of the LNP composition; a sterol is cholesterol in a total amount of 35-45 mol % of the total lipid content of the LNP composition; the total amount of phospholipid of 7-15 mol % of the total lipid content of the LNP composition; the one or more phospholipids consist of DSPC and the PS lipid is one or more lipids selected from the group consisting of the L-serine configuration of DPPS and DSPS; and the total amount of the PS lipid is about 5 mol % of the total lipid content of the LNP composition.

The present disclosure also provides the following first set of additional numbered embodiments, and all combinations thereof:

1. A lipid nanoparticle (LNP) composition comprising a TLR-activating nucleic acid.

2. The composition of embodiment 1, wherein the TLR-activating nucleic acid is a RNA oligonucleotide or a DNA oligonucleotide.

3. The composition of embodiment 1, wherein the TLR-activating nucleic acid is a TLR9-activating DNA oligonucleotide and/or a TLR7/8 activating RNA oligonucleotide agonist.

4. The composition of embodiment 1, further comprising a TLR4 agonist.

5. The composition of embodiment 4, wherein the TLR4 agonist is selected from the group consisting of Monophosphoryl Lipid A (MPL), Monophosphoryl Lipid A-504, Monophosphoryl 3-Deacyl Lipid A, and Monophosphoryl Hexa-acyl Lipid A, and 3-Deacyl.

6. The composition of embodiment 1, wherein the TLR-activating nucleic acid is a TLR9-activating oligonucleotide, optionally CPG1018.

7. The composition of embodiment 1, wherein the TLR-activating nucleic acid comprises a TLR7 and/or TLR-8 activating RNA oligonucleotide.

8. The composition of embodiment 1, wherein the TLR-activating nucleic acid comprises both a TLR-activating DNA oligonucleotide and a TLR-activating RNA oligonucleotide

9. The composition of embodiment 1, wherein the TLR-activating nucleic acid is a DNA comprising a CpG site.

10. The composition of embodiment 9, wherein the TLR-activating nucleic acid activates human TLR9.

11. The composition of embodiment 10, wherein the CpG is selected from the group consisting of: CpG-ODN 1018 (SEQ ID NO. 2), CpG-ODN 2006 (SEQ ID NO. 3; also known as ODN 7909 or PF3512676), CpG-ODN 2216 (SEQ ID NO. 6), CpG-ODN 2336 (SEQ ID NO. 7) and CpG-ODN 2395 (SEQ ID NO. 8)

12. The composition of embodiment 1, wherein the TLR-activating nucleic acid comprises a TLR7 and/or TLR-8 activating nucleic acid selected from the group consisting of: R-0006 (SEQ ID NO. 25), R-1075 (SEQ ID NO. 11), 9.2dr (SEQ ID NO. 212), 9.3as (SEQ ID NO. 113), 9.2s (SEQ ID NO. 114) and HIV sequence 1 (SEQ ID NO. 116).

13. The composition of embodiment 1, wherein the TLR-activating nucleic acid activates RIG-I.

14. The composition of embodiment 13, wherein the TLR-activating nucleic acid is selected from the group consisting of: 3P-GFP2 (SEQ ID NO. 127) annealed with either SEQ ID NO. 128 or SEQ ID NO. 129, 3P-A24 (SEQ ID NO. 130) annealed with either SEQ ID NO. 133 or SEQ ID NO. 134, 3P-G24 (SEQ ID NO. 130) annealed with either SEQ ID NO. 140 or SEQ ID NO. 141, 3P-C₂₄ (SEQ ID NO. 130) annealed with either SEQ ID NO. 147 or SEQ ID NO. 148 and 3P-U24 (SEQ ID NO. 151) annealed with either SEQ ID NO. 154 or SEQ ID NO. 155.

15. A lipid nanoparticle (LNP) composition, comprising or consisting of:
a. the TLR-activating nucleic acid of any one of embodiments 1-14;
b. an ionizable cationic lipid comprising an ionizable cationic lipid at a N/P ratio of 4 to 6 relative to the mRNA, the ionizable cationic lipid present in the LNP composition in a total amount of 46-54 mol % of a total lipid content of the LNP composition;
c. one or more phospholipids selected from the group consisting of distearoylphosphatidylcholine (DSPC), hydrogenated soy phosphatidylcholine (HSPC), and dipalmitoylphosphatidylcholine (DPPC), in a total amount of 10-18 mol % of the total lipid content of the LNP composition;
d. one or more anionic phospholipids selected from the group consisting of dipalmitoylphosphatidyl-L-serine (DPPS), or distearoylphosphatidyl-L-serine (DSPS), distearoylphosphatidylglycerol (DSPG), and dipalmitoyphosphatidylglycerol (DPPG) in a total amount of 2-8 mol % of the total lipid content of the LNP composition;
e. PEG(2000)-dimyristoylglycerol (PEG-DMG) in a total amount of 1-3.5 mol % of the total lipid content of the LNP composition; and
f. cholesterol.

16. A lipid nanoparticle (LNP) composition comprising:
a. the TLR-activating nucleic acid of any one of embodiments 1-14;
b. an ionizable cationic lipid comprising a KC3 ionizable cationic lipid at a N/P ratio of 3 to 7 relative to the nucleic acid, the ionizable cationic lipid present in the LNP composition in a total amount of 46-54 mol % of a total lipid content of the LNP composition;
c. one or more phospholipids in a total amount of 5-20 mol % of the total lipid content of the LNP composition;
d. one or more anionic phospholipids in a total amount of 2-8 mol % of the total lipid content of the LNP composition;
e. a conjugated lipid in a total amount of 1-3.5 mol % of the total lipid content of the LNP composition; and f. a sterol.

17. The composition of embodiments 16, wherein the one or more anionic phospholipids is a phosphatidylserine (PS) or phosphatidylglycerol (PG).

18. The composition of embodiments 17, wherein the one or more anionic phospholipids is selected from the group consisting of: dipalmitoylphosphatidyl-L-serine (DPPS), or distearoylphosphatidyl-L-serine (DSPS), distearoylphosphatidylglycerol (DSPG), and dipalmitoyphosphatidylglycerol (DPPG).

19. The composition of any one of embodiments 15-18, wherein the one or more phospholipids comprises distearoylphosphatidylcholine (DSPC), hydrogenated soy phosphatidylcholine (HSPC), dipalmitoylphosphatidylcholine (DPPC) or a combination thereof.

20. The composition of embodiment 19, wherein the conjugated lipid is PEG(2000)-dimyristoylglycerol (PEG-DMG).

21. The composition of embodiment 20, wherein the sterol is cholesterol.

22. The composition of any one of embodiments 15-21, wherein the ionizable cationic lipid comprises 3-((S)-2,2-di((Z)-octadec-9-en-1-yl)-1,3-dioxolan-4-yl)-N,N-dimethylpropan-1-amine (KC3-OA).

23. The composition of any one of embodiments 15-21, wherein the ionizable cationic lipid further comprises a KC4 ionizable cationic lipid.

24. The composition of any one of embodiments 15-21, wherein the ionizable cationic lipid is 4-rac-2,2-di((Z)-octadec-9-en-1-yl)-1,3-dioxolan-4-yl)-N,N-dimethylbutan-1-amine (AKG-KC4-OA).

25. The composition of any one of embodiments 15-22, wherein the composition consists of:
a. 48 mol % KC3-OA;
b. 5 mol % DPPS or DSPG;
c. 5-10 mol % DSPC or HSPC;
d. 1.5 mol % PEG-DMG; and
e. 35.5-40.5 mol % cholesterol.

26. A lipid nanoparticle (LNP) composition comprising:
a. the TLR-activating nucleic acid of any one of embodiments 1-14;
b. an ionizable cationic lipid comprising a KC3 ionizable cationic lipid at a N/P ratio of 3 to 7 relative to the nucleic acid, the ionizable cationic lipid present in the LNP composition in a total amount of 46-54 mol % of a total lipid content of the LNP composition;
c. one or more phospholipids in a total amount of 5-20 mol % of the total lipid content of the LNP composition;
d. one or more anionic phospholipids in a total amount of 2-8 mol % of the total lipid content of the LNP composition;
e. a conjugated lipid in a total amount of 1-3.5 mol % of the total lipid content of the LNP composition; and
f. a sterol.

27. The composition of embodiment 26, wherein the composition comprises cholesterol in a total amount of 35.5-42.7 mol % of total lipid in the LNP composition.

28. The composition of any one of embodiments 26-27, wherein the composition comprises 48 mol % of a KC3 ionizable cationic lipid, 40.5 mol % cholesterol, and 5 mol % (L-Serine) DPPS lipid, wherein each mol % refers to the mol % of the total lipid content of the LNP composition.

29. The composition of any one of embodiments 26-27, wherein the composition comprises 48 mol % of a KC3 ionizable cationic lipid, 38.5 mol % cholesterol, and 5 mol % (L-Serine) DPPS lipid, wherein each mol % refers to the mol % of the total lipid content of the LNP composition.

30. the composition of any one of embodiments 26-27, wherein the composition comprises 45 mol % of a KC3 ionizable cationic lipid, 42.7 mol % cholesterol, and 5 mol % (L-Serine) DPPS lipid, wherein each mol % refers to the mol % of the total lipid content of the LNP composition.

31. The composition of any one of embodiments 26-27, wherein the composition comprises 50 mol % of a KC3 ionizable cationic lipid, 38.5 mol % cholesterol, 5 mol % (L-Serine) DPPS lipid, and a total of 10 mol % phospholipid concentration, wherein each mol % refers to the mol % of the total lipid content of the LNP composition.

32. The composition of any one of embodiments 26-27, wherein the composition comprises 48 mol % of a KC3 ionizable cationic lipid, 40.5 mol % cholesterol, 5 mol % (L-Serine) DPPS lipid, and a total of 10 mol % phospholipid concentration, wherein each mol % refers to the mol % of the total lipid content of the LNP composition.

33. The composition of any one of embodiments 26-27, wherein the composition comprises 48 mol % of a KC3 ionizable cationic lipid, 40.5 mol % cholesterol, 5 mol % (L-Serine) DPPS lipid, 5 mol % DSPC or DPPC; and a total of 10 mol % phospholipid concentration, wherein each mol % refers to the mol % of the total lipid content of the LNP composition.

34. The composition of any one of embodiments 26-27, wherein the composition comprises 46.5 mol % of a KC3 ionizable cationic lipid, 42 mol % cholesterol, 5 mol % (L-Serine) DPPS lipid, wherein each mol % refers to the mol % of the total lipid content of the LNP composition.

35. The composition of any one of embodiments 26-27, wherein the composition comprises mol % total phospholipid and 35.5 mol % cholesterol.

36. The composition of any one of embodiments 26-27, wherein the composition comprises mol % total phospholipid and 40.5 mol % cholesterol.

37. The composition of any one of embodiments 26-27, wherein the composition comprises 40.5 mol % cholesterol, 5% anionic lipid (DPPS) and 5% PC (DSPC or DPPC) and a total of 10 mol % phospholipid concentration.

38. The composition of any one of embodiments 26-27, wherein the composition comprises 48 mol % cationic ionizable lipid, 5 mol % PC (DPPC), 5 mol % anionic lipid (DPPS), 40.5 mol % cholesterol, 1.5 mol % conjugated lipid (PEG-DMG).

39. The composition of any one of embodiments 1-38, wherein the composition is a vaccine.

40. A pharmaceutical composition comprising the LNP composition of any one of embodiments 1-38, and a pharmaceutically acceptable carrier.

The present disclosure also provides the following second set of additional numbered embodiments, and all combinations thereof:

1. A lipid nanoparticle (LNP) composition comprising a TLR-activating nucleic acid.

2. The composition of embodiment 1, wherein the TLR-activating nucleic acid is a RNA oligonucleotide or a DNA oligonucleotide.

3. The composition of embodiment 1, wherein the TLR-activating nucleic acid is a TLR9-activating DNA oligonucleotide and/or a TLR7/8-activating RNA oligonucleotide.

4. The composition of embodiment 1, further comprising a TLR4 agonist.

5. The composition of embodiment 4, wherein the TLR4 agonist is selected from the group consisting of Monophosphoryl Lipid A (MPL), Monophosphoryl Lipid A-504, Monophosphoryl 3-Deacyl Lipid A, and Monophosphoryl Hexa-acyl Lipid A, and 3-Deacyl.

6. The composition of embodiment 1, wherein the TLR-activating nucleic acid is a TLR9-activating oligonucleotide, optionally CPG1018.

7. The composition of embodiment 1, wherein the TLR-activating nucleic acid comprises a TLR7 and/or TLR-8 activating RNA oligonucleotide.

8. The composition of embodiment 1, wherein the TLR-activating nucleic acid comprises both a TLR-activating DNA oligonucleotide and a TLR-activating RNA oligonucleotide

9. The composition of embodiment 1, wherein the TLR-activating nucleic acid is a DNA comprising a CpG site.

10. The composition of embodiment 9, wherein the TLR-activating nucleic acid activates human TLR9.

11. The composition of embodiment 10, wherein the CpG is selected from the group consisting of: CpG-ODN 1018 (SEQ ID NO. 2), CpG-ODN 2006 (SEQ ID NO. 3; also known as ODN 7909 or PF3512676), CpG-ODN 2216 (SEQ ID NO. 6), CpG-ODN 2336 (SEQ ID NO. 7) and CpG-ODN 2395 (SEQ ID NO. 8)

12. The composition of embodiment 1, wherein the TLR-activating nucleic acid comprises a TLR7 and/or TLR-8 activating nucleic acid selected from the group consisting of: R-0006 (SEQ ID NO. 25), R-1075 (SEQ ID NO. 11), 9.2dr (SEQ ID NO. 212), 9.3as (SEQ ID NO. 113), 9.2s (SEQ ID NO. 114) and HIV sequence 1 (SEQ ID NO. 116).

13. The composition of embodiment 1, wherein the TLR-activating nucleic acid activates RIG-I.

14. The composition of embodiment 13, wherein the TLR-activating nucleic acid is selected from the group consisting of: 3P-GFP2 (SEQ ID NO. 127) annealed with either SEQ ID NO. 128 or SEQ ID NO. 129, 3P-A24 (SEQ ID NO. 130) annealed with either SEQ ID NO. 133 or SEQ ID NO. 134, 3P-G24 (SEQ ID NO. 130) annealed with either SEQ ID NO. 140 or SEQ ID NO. 141, 3P-C₂₄ (SEQ ID NO. 130) annealed with either SEQ ID NO. 147 or SEQ ID NO. 148 and 3P-U24 (SEQ ID NO. 151) annealed with either SEQ ID NO. 154 or SEQ ID NO. 155.

15. A lipid nanoparticle (LNP) composition consisting of:
a. a nucleic acid antigen recognized by a germline-encoded pattern recognition receptor (PRR);
   b. an ionizable cationic lipid comprising an ionizable cationic lipid at a N/P ratio of 4 to 6 relative to the mRNA, the ionizable cationic lipid present in the LNP composition in a total amount of 46-54 mol % of a total lipid content of the LNP composition;
   c. one or more phospholipids selected from the group consisting of distearoylphosphatidylcholine (DSPC), hydrogenated soy phosphatidylcholine (HSPC), and dipalmitoylphosphatidylcholine (DPPC), in a total amount of 10-18 mol % of the total lipid content of the LNP composition;
   d. one or more anionic phospholipids selected from the group consisting of dipalmitoylphosphatidyl-L-serine (DPPS), or distearoylphosphatidyl-L-serine (DSPS), distearoylphosphatidylglycerol (DSPG), and dipalmitoyphosphatidylglycerol (DPPG) in a total amount of 2-8 mol % of the total lipid content of the LNP composition;
   e. PEG(2000)-dimyristoylglycerol (PEG-DMG) in a total amount of 1-3.5 mol % of the total lipid content of the LNP composition; and
   f. cholesterol.

16. The composition of embodiment 15, wherein the PRR recognizes a conserved microbial pathogen-associated molecular patterns (PAMPs) and danger-associated molecular patterns (DAMPs).

17. The composition of embodiment 16, wherein the nucleic acid antigen is a CpG oligodeoxynucleotide recognized by TLR9.

18. The composition of embodiment 16, wherein the nucleic acid antigen is a ssRNA oligonucleotides recognized by TLR7 or TLR8.

19. The composition of embodiment 16, wherein the nucleic acid antigen is a ssRNA oligonucleotides recognized by TLR7 and TLR8.

20. The composition of embodiment 16, wherein the nucleic acid antigen is a RIG-I recognizes uncapped 5′ triphosphate RNA (aka pppRNA, 3pRNA).

21. The composition of embodiment 20, wherein the nucleic acid antigen is a 5′ppp ssRNA with poly-U/UG, poly-U/UC, poly-A/AG.

22. The composition of embodiment 21, wherein the nucleic acid antigen comprises RNA regions enriched with polyuridine and interspersed guanosines (poly-U/UG), polyuridine with interspersed cytidines, polyadenosine interspersed with guanosines.

23. The composition of embodiment 16, wherein the nucleic acid antigen encodes MDA5 (melanoma differentiation-associated protein 5).

24. The composition of embodiment 16, wherein the nucleic acid antigen is selected from the group consisting of NOD2, LGP2, DDX1-DDX21-DHX36, DDX60, DHX9, DHX36, DDX41, AIM2, IF16, ZBP1, LRRFIP1, and STING.

25. The composition of embodiment 16, wherein the nucleic acid antigen is a CpG oligodeoxynucleotide (ODN) 1018 (SEQ ID NO: 2).

26. The composition of embodiment 16, wherein the nucleic acid antigen is a CpG-ODN 2006 (SEQ ID NO: 3).

27. The composition of embodiment 16, wherein the nucleic acid antigen is TLR7/8 ssRNA oligonucleotide 5′-UUGUUGUUGUUGUUGUUGUU-3′ (SEQ ID NO: 25).

28. The composition of embodiment 16, wherein the nucleic acid antigen is mRNA encoding for SARS-CoV-2 Spike protein (SEQ ID NO: 1).

29. The composition of embodiment 16, wherein the nucleic acid antigen is selected from the group consisting of SEQ ID NO:1, SEQ ID NO:2, or SEQ ID NO:3.

30. The composition of embodiment 16, wherein the nucleic acid antigen is a TLR-9 CpG-ODN adjuvant.

31. The composition of embodiment 16, wherein the nucleic acid antigen is selected from the group consisting of SEQ ID NO:11-SEQ ID NO:117 (Table 55).

32. The composition of embodiment 2, wherein the nucleic acid is selected from the group consisting of SEQ ID NO:118-SEQ ID NO:126 (Table 56) or SEQ ID NO:127-167 (Table 60).

33. The lipid nanoparticle (LNP) composition of any one of embodiments 1-32, comprising:
a. the TLR-activating nucleic acid antigen;
b. an ionizable cationic lipid comprising a KC3 ionizable cationic lipid at a N/P ratio of 3 to 7 relative to the nucleic acid, the ionizable cationic lipid present in the LNP composition in a total amount of 46-54 mol % of a total lipid content of the LNP composition;
c. one or more phospholipids in a total amount of 5-20 mol % of the total lipid content of the LNP composition;
d. one or more anionic phospholipids in a total amount of 2-8 mol % of the total lipid content of the LNP composition;
e. a conjugated lipid in a total amount of 1-3.5 mol % of the total lipid content of the LNP composition; and
f. a sterol.

34. The composition of embodiment 33, wherein the one or more anionic phospholipids is a phosphatidylserine (PS) or phosphatidylglycerol (PG).

35. The composition of embodiment 34, wherein the one or more anionic phospholipids is selected from the group consisting of: dipalmitoylphosphatidyl-L-serine (DPPS), or distearoylphosphatidyl-L-serine (DSPS), distearoylphosphatidylglycerol (DSPG), and dipalmitoyphosphatidylglycerol (DPPG).

36. The composition of embodiment 33, wherein the one or more phospholipids comprises distearoylphosphatidylcholine (DSPC), hydrogenated soy phosphatidylcholine (HSPC), dipalmitoylphosphatidylcholine (DPPC) or a combination thereof.

37. The composition of embodiment 36, wherein the conjugated lipid is PEG(2000)-dimyristoylglycerol (PEG-DMG).

38. The composition of embodiment 37, wherein the sterol is cholesterol.

39. The composition of embodiment 38, wherein the ionizable cationic lipid comprises 3-((S)-2,2-di((Z)-octadec-9-en-1-yl)-1,3-dioxolan-4-yl)-N,N-dimethylpropan-1-amine (KC3-OA).

40. The composition of embodiment 39, wherein the ionizable cationic lipid further comprises a DM2 ionizable cationic lipid.

41. The composition of embodiment 40, wherein the ionizable cationic lipid is 4-rac-2,2-di((Z)-octadec-9-en-1-yl)-1,3-dioxolan-4-yl)-N,N-dimethylbutan-1-amine (AKG-KC4-OA).

42. The composition of embodiment 38, wherein the composition consists of:
a. 48 mol % KC3-OA;
b. 5 mol % DPPS or DSPG;
c. 5-10 mol % DSPC or HSPC;
d. 1.5 mol % PEG-DMG; and
e. 35.5-40.5 mol % cholesterol.

43. The composition of any one of embodiments 33-42, wherein the ionizable cationic lipid is KC3-OA, KC3-PA, KC3-01, KC3-C17 (8:1), or KC3-C15 (C8:1).

44. The composition of any one of embodiments 1-14, wherein the lipid nanoparticle (LNP) composition comprises:
a. the TLR-activating nucleic acid antigen;
b. an ionizable cationic lipid comprising a KC3 ionizable cationic lipid at a N/P ratio of 3 to 7 relative to the nucleic acid, the ionizable cationic lipid present in the LNP composition in a total amount of 46-54 mol % of a total lipid content of the LNP composition;
c. one or more phospholipids in a total amount of 5-20 mol % of the total lipid content of the LNP composition;
d. one or more anionic phospholipids in a total amount of 2-8 mol % of the total lipid content of the LNP composition;
e. a conjugated lipid in a total amount of 1-3.5 mol % of the total lipid content of the LNP composition; and
f. a sterol.

45. The composition of embodiment 44, wherein the nucleic acid antigen has at least 90% identity to a polynucleotide sequence selected from the group consisting of: SEQ ID NO:11-SEQ ID NO:126.

46. The composition of any one of embodiments 1-45, wherein the composition is a vaccine.

47. A pharmaceutical composition comprising the lipid nanoparticle of any one of embodiments 1-45, and a pharmaceutically acceptable carrier.

While this disclosure has been described in relation to certain embodiments, and many details have been set forth for purposes of illustration, it will be apparent to those skilled in the art that this disclosure includes additional embodiments, and that some of the details described herein may be varied considerably without departing from this disclosure. This disclosure includes such additional embodiments, modifications and equivalents. In particular, this disclosure includes any combination of the features, terms, or elements of the various illustrative components and examples.

### (VII) EXAMPLES

Unless explicitly indicated otherwise, the isomer form of the phosphatidylserine lipids used in the Examples is phosphatidyl-L-serine.

Certain examples are provided below to illustrate various embodiments of the embodiments disclosed herein. One of ordinary skill in the art will recognize that the various embodiments disclosed herein are not limited to these specific illustrative examples.

### Example 1A. Synthesis of Ionizable Lipids

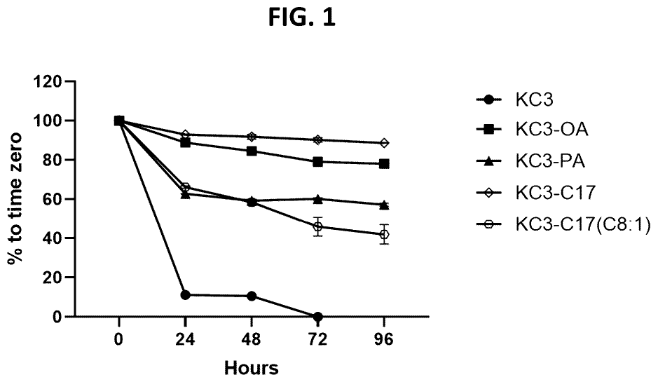

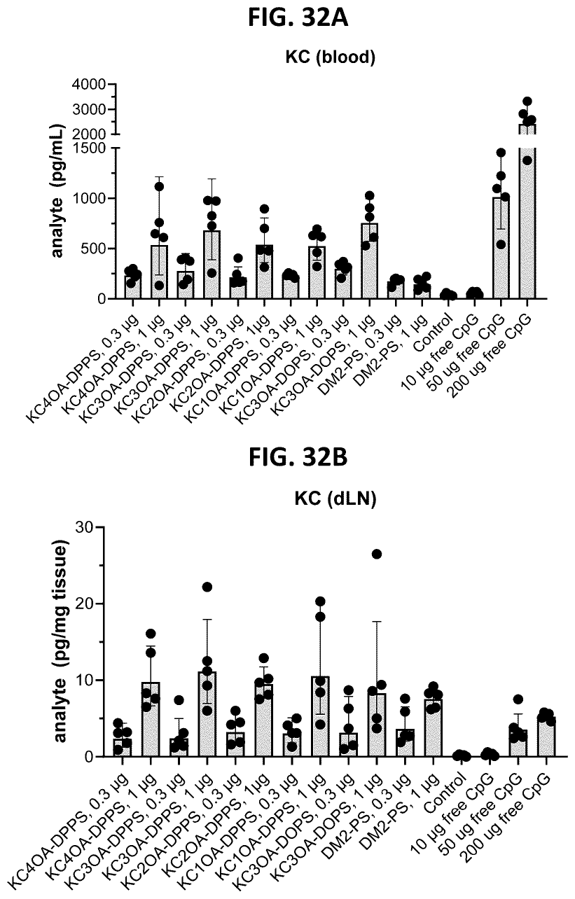

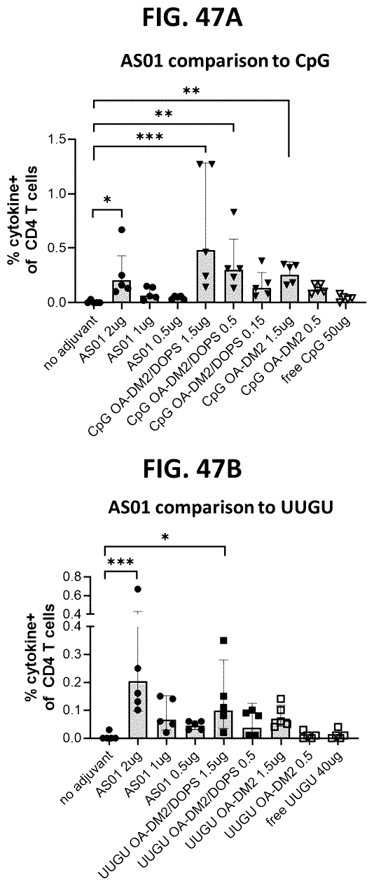

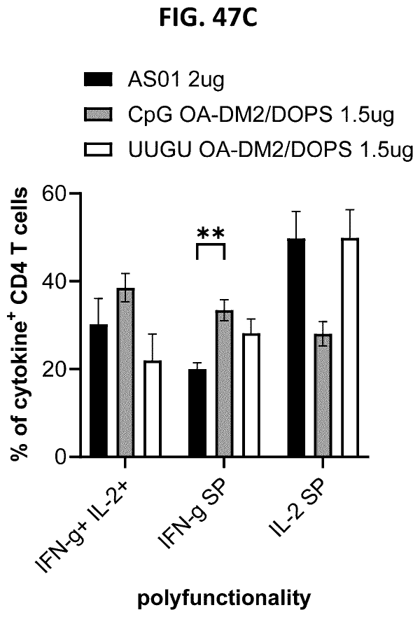

The chemical synthesis of ionizable cationic lipids disclosed in WO2022/115645 (Examples 1A and 1B, and FIG. 1), WO2023/230587 (Examples 1-Examples 1A-1E, and FIGS. 47A-51), and WO2024/138121 (Example 1 and FIGS. 32, 33, and 34), which disclosure from each of these documents is incorporated herein by reference in its entirety.

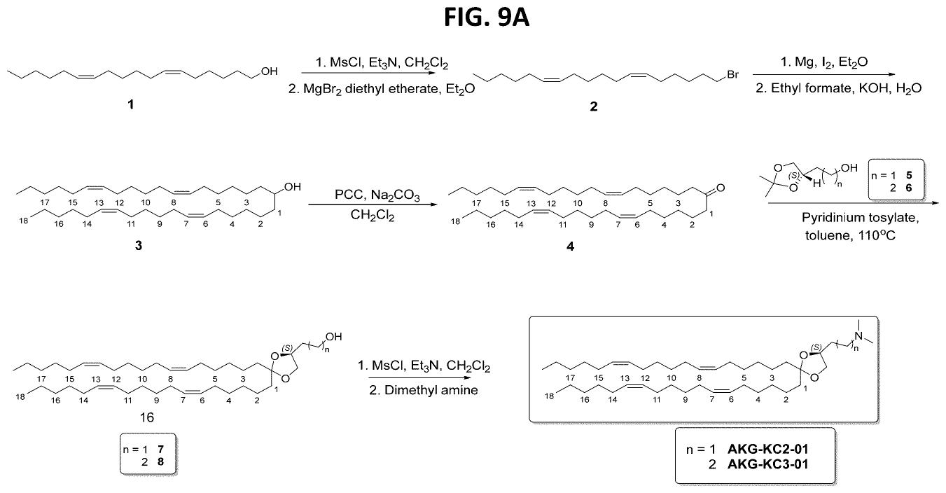

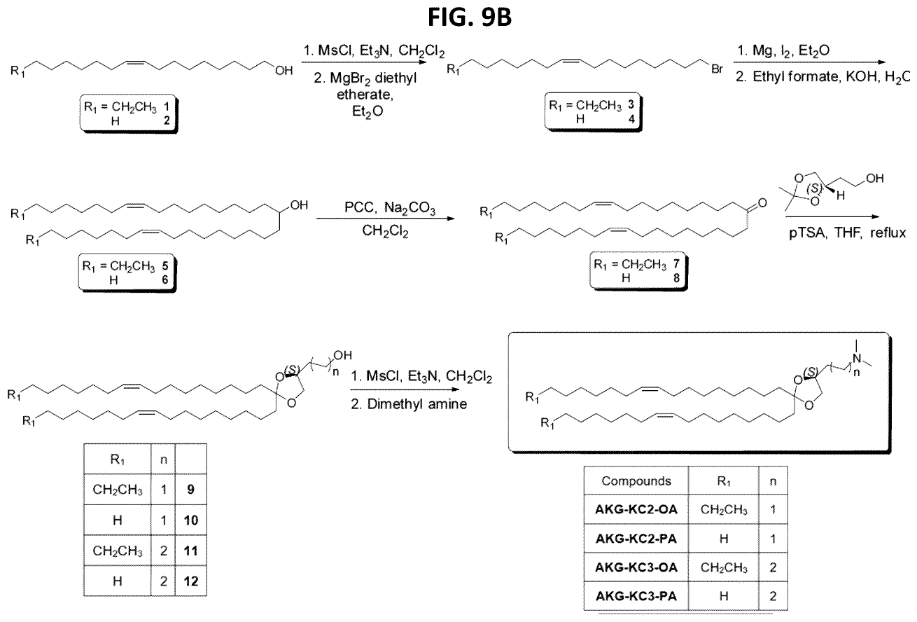

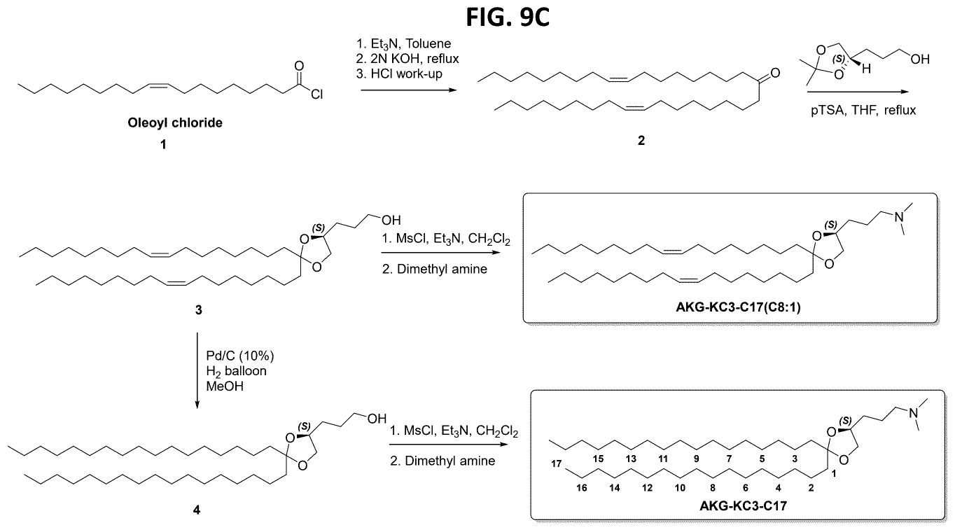

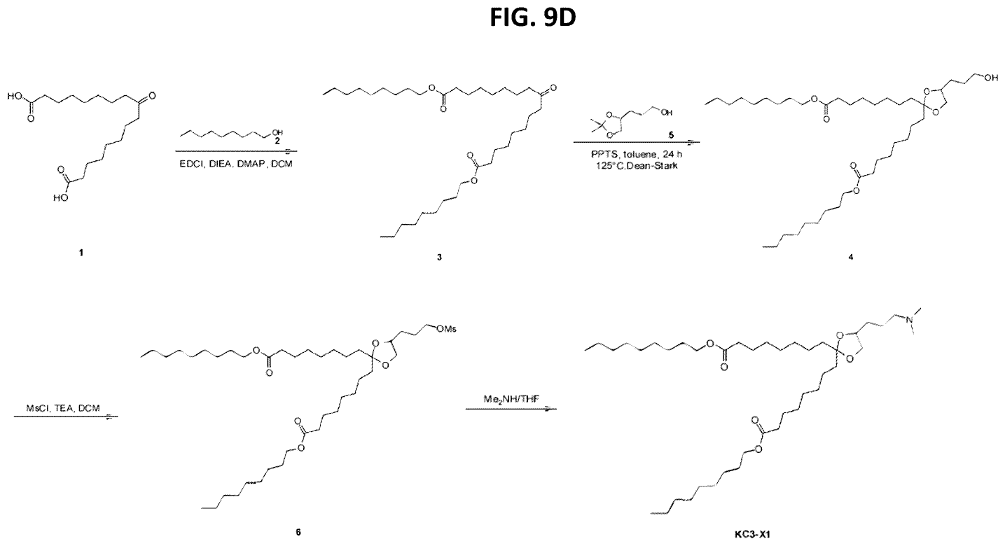

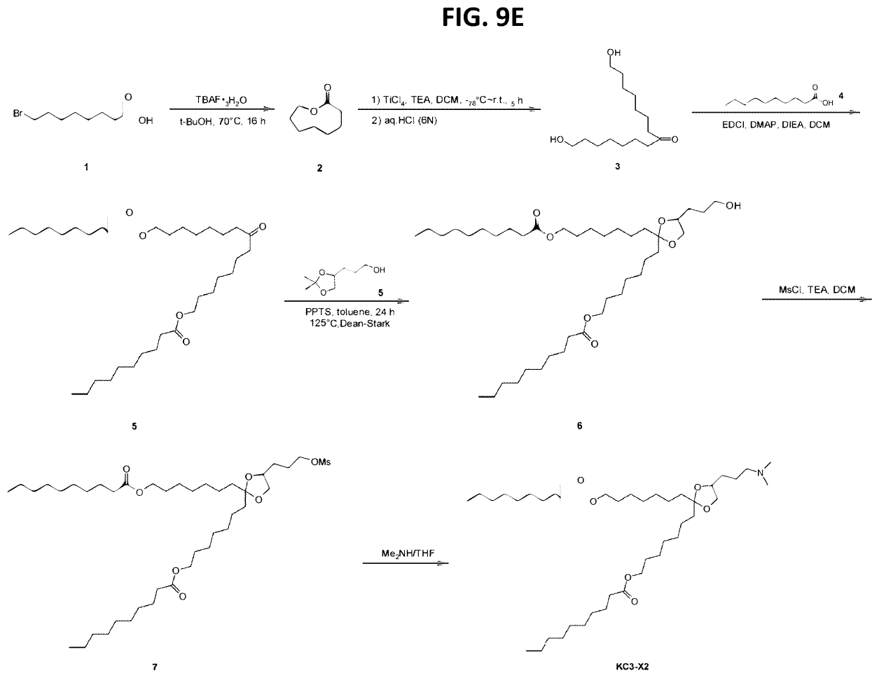

Chemical synthesis schemes for the preparation of various ionizable cationic lipids are described herein in FIG. 9A, FIG. 9B, FIG. 9C, FIG. 9D, and FIG. 9E, and in the reaction methods below.
1. 2-((S)-2,2-di((6Z,12Z)-octadeca-6,12-dien-1-yl)-1,3-dioxolan-4-yl)-N,N-dimethylethan-1-amine (AKG-KC2-01, O-12095)
2. 3-((S)-2,2-di((6Z,12Z)-octadeca-6,12-dien-1-yl)-1,3-dioxolan-4-yl)-N,N-dimethylpropan-1-amine (AKG-KC3-01, O-12096)
3. 2-((S)-2,2-di((Z)-octadec-9-en-1-yl)-1,3-dioxolan-4-yl)-N,N-dimethylethan-1-amine (AKG-KC2-OA, O-11880)
4. 2-((S)-2,2-di((Z)-hexadec-9-en-1-yl)-1,3-dioxolan-4-yl)-N,N-dimethylethan-1-amine (AKG-KC2-PA, O-11879)
5. 3-((S)-2,2-di((Z)-octadec-9-en-1-yl)-1,3-dioxolan-4-yl)-N,N-dimethylpropan-1-amine (AKG-KC3-OA, O-11957)
6. 3-((S)-2,2-di((Z)-hexadec-9-en-1-yl)-1,3-dioxolan-4-yl)-N,N-dimethylpropan-1-amine (AKG-KC3-PA, O-12418)
7. 3-((S)-2,2-di((Z)-heptadec-8-en-1-yl)-1,3-dioxolan-4-yl)-N,N-dimethylpropan-1-amine (AKG-KC3-C17(C8:1))
8. (S)-3-(2,2-diheptadecyl-1,3-dioxolan-4-yl)-N,N-dimethylpropan-1-amine (AKG-KC3-C17)

### Synthesis of 2-((S)-2,2-di((6Z,12Z)-octadeca-6,12-dien-1-yl)-1,3-dioxolan-4-yl)-N,N-dimethylethan-1-amine (AKG-KC2-01, O-12095)

### 3-((S)-2,2-di((6Z,12Z)-octadeca-6,12-dien-1-yl)-1,3-dioxolan-4-yl)-N,N-dimethylpropan-1-amine (AKG-KC3-01, O-12096)

FIG. 9A is a scheme showing the synthesis of AKG-KC2-01 and AKG-KC3-01.

### Experimental Procedure

### Synthesis of (6Z,12Z)-1-bromooctadeca-6,12-diene, 2

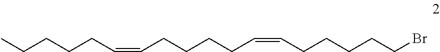 [SMILES: CCCCC/C=C\CCCC/C=C\CCCCCBr]

To a solution of (6Z,12Z)-octadeca-6,12-dien-1-ol, 1 (3.6 g, 13.7 mmol) in dichloromethane (50 mL) at 0° C. was added methane sulfonyl chloride (1.26 mL, 16.4 mmol) and triethylamine (3.6 mL, 20.5 mmol). The resulting solution was warmed to room temperature and stirred for 2 hours. The mixture was quenched with water and extracted with dichloromethane (2×100 mL). The combined organics were washed with brine then dried over magnesium sulfate then filtered. The filtrate was concentrated under vacuum to give a crude oil. The resulting oil was dissolved in diethyl ether (50 mL), added to a stirring slurry of magnesium bromide ethyl etherate (7 g, 27.4 mmol) in diethyl ether (50 mL) at OC. The mixture was warmed to room temperature and stirred for 2 hours. The reaction mixture was quenched with water and extracted with ethyl acetate (2×100 mL). The combined organics were washed with brine then dried over magnesium sulfate then filtered. The filtrate was concentrated under vacuum to give a crude oil. The crude oil was purified by chromatography on silica using 5-10% ethyl acetate in n-hexane as eluant to give (6Z,12Z)-1-bromooctadeca-6,12-diene, 3 (2.9 g, 8.89 mmol, 65%) as a yellow oil.

¹H NMR (300 MHz, CDCl₃): 5.36-5.33 (m, 4H), 3.42-3.37 (t, J=7.5 Hz, 2H), 2.04-1.97 (m, 8H), 1.83-1.83 (m, 2H), 1.37-1.28 (m, 14H), 0.90-0.86 (t, J=6.6 Hz, 3H).

### Synthesis of (6Z,12Z,25Z,31Z)-heptatriaconta-6,12,25,31-tetraen-19-ol, 3

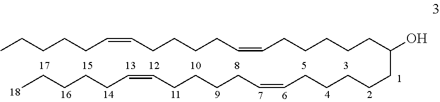 [SMILES: CCCCC/C=C\CCCC/C=C\CCCCCC(O)CCCCC/C=C\CCCC/C=C\CCCCC]

A solution of (6Z,12Z)-1-bromooctadeca-6,12-diene, 2 (2 g, 6.08 mmol) in ether (10 mL) was added to a mixture of magnesium turnings (162 mg, 6.69 mmol) and iodine in ether (2 mL) under argon at room temperature. The mixture stirred at room temperature for 90 minutes (magnesium turnings consumed) whereupon ethyl formate (0.24 mL, 3.04 mmol) was added. After stirring for one hour at room temperature, the reaction was quenched with 1N HCl solution. The mixture was extracted with ethyl acetate (2×100 mL) and the combined organics washed with water then brine. The organics were dried under magnesium sulfate, filtered, and the filtrate concentrated under vacuum to give a crude oil. The resulting oil was dissolved in ethanol (10 mL) and added to a solution of potassium hydroxide (260 mg) in water (3 mL). After stirring for 12 hours, the mixture pH was adjusted 4 with 2N HCl. The aqueous solution was extracted with dichloromethane (2×) and combined. The organics were washed with brine then dried under magnesium sulfate and filtered. The filtrate was concentrated under vacuum to give a crude oil. Purification of the crude oil on silica using 10-30% ethyl acetate in n-hexane as eluant to give (6Z,12Z,25Z,31Z)-heptatriaconta-6,12,25,31-tetraen-19-ol, 3 (0.29 g, 0.55 mmol, 18%) as a clear oil.

¹H NMR (300 MHz, CDCl₃): 5.36-5.32 (m, 8H), 3.57 (bs, 1H), 3.33-3.32, (m, 2H), 2.13-1.97 (m, 16H), 1.36-1.29 (m, 34H), 0.90-0.86 (t, J=6.6 Hz, 6H).

### Synthesis of (6Z,12Z,25Z,31Z)-heptatriaconta-6,12,25,31-tetraen-19-one, 4

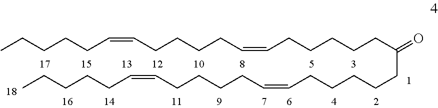 [SMILES: CCCCC/C=C\CCCC/C=C\CCCCCC(=O)CCCCC/C=C\CCCC/C=C\CCCCC]

To a mixture of (6Z,12Z,25Z,31Z)-heptatriaconta-6,12,25,31-tetraen-19-ol, 3 (0.29 g, 0.55 mmol) and sodium carbonate (3 mg, 0.03 mmol) in dichloromethane was added pyridinium chlorochromate (236 mg, 1.1 mmol) at 0° C. The mixture was warmed to room temperature and stirred for one hour. After one hour, silica gel (1 g) was added to reaction and the mixture filtered. The filtrate was concentrated, and the resulted oil purified on silica using 10-20% ethyl acetate in n-hexane as eluant to give (6Z,12Z,25Z,31Z)-heptatriaconta-6,12,25,31-tetraen-19-one, 4 (0.12 g, 0.23 mmol, 42%) as a clear oil.

¹H NMR (300 MHz, CDCl₃): 5.36-5.32 (m, 8H), 3.36-3.32, (m, 1H), 2.40-2.35 (t, J=6.6 Hz, 3H), 2.14-2.00 (m, 16H), 1.58-1.54 (m, 4H), 1.34-1.29 (m, 28H), 0.90-0.86 (t, J=6.6 Hz, 6H).

### Synthesis of 2-((S)-2,2-di((9Z,12Z)-octadeca-9,12-dien-1-yl)-1,3-dioxolan-4-yl) ethan-1-ol, 7

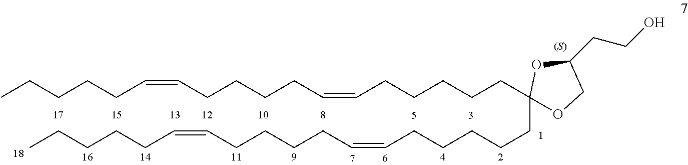

A mixture of (6Z,12Z,25Z,31Z)-heptatriaconta-6,12,25,31-tetraen-19-one, 4 (0.12 g, 0.23 mmol), (4S)-(+)-4-(2-hydroxyethyl)-2,2-dimethyl-1,3-dioxolane 5 (0.20 g, 1.38 mmol), and pyridinium p-toluene sulfonate (9 mg) in toluene (10 mL) was heated at reflux under nitrogen positive pressure. After 12 hours, the mixture was concentrated under vacuum to give a crude oil. The resulting crude oil was purified by chromatography on silica using 20-40% ethyl acetate in n-hexane as eluant to give 2-((S)-2,2-di((9Z,12Z)-octadeca-9,12-dien-1-yl)-1,3-dioxolan-4-yl) ethan-1-ol, 7 (0.11 g, 0.17 mmol, 77%) as a clear oil.

¹H NMR (300 MHz, CDCl₃): 5.36-5.32 (m, 8H), 4.25-4.20 (m, 1H), 4.10-4.06 (m, 1H), 3.82-3.77 (m, 1H), 3.54-3.49 (m, 1H), 2.23-2.19 (t, J=6.6 Hz, 3H), 2.14-2.00 (m, 16H), 1.84-1.78 (m, 2H), 1.62-1.51 (m, 6H), 1.34-1.29 (m, 28H), 0.90-0.86 (t, J=6.6 Hz, 6H).

### Synthesis of 3-((S)-2,2-di((9Z,12Z)-octadeca-9,12-dien-1-yl)-1,3-dioxolan-4-yl)propan-1-ol, 8

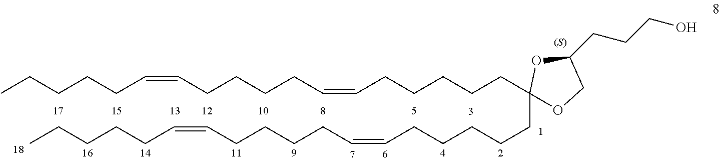

A mixture of (6Z,12Z,25Z,31Z)-heptatriaconta-6,12,25,31-tetraen-19-one, 4 (0.50 g, 0.95 mmol), (S)-(3)-(2,2-Dimethyl-1,3-dioxolane-4-yl)propanol 6 (0.76 g, 4.75 mmol), and pyridinium p-toluene sulfonate (36 mg) in toluene (10 mL) was heated at reflux under nitrogen positive pressure. After 12 hours, the mixture was concentrated under vacuum to give a crude oil. The resulting crude oil was purified by chromatography on silica using 20-40% ethyl acetate in n-hexane as eluant to 3-((S)-2,2-di((9Z,12Z)-octadeca-9,12-dien-1-yl)-1,3-dioxolan-4-yl)propan-1-ol, 8 (0.48 g, 0.76 mmol, 80%) as a clear oil.

¹H NMR (300 MHz, CDCl₃): 5.34-5.29 (m, 8H), 4.06-4.02 (m, 2H), 3.67-3.47 (m, 2H), 3.45-3.43 (m, 1H), 2.12-2.01 (m, 16H), 1.65-1.62 (m, 8H), 1.34-1.29 (m, 32H), 0.89-0.85 (t, J=6.6 Hz, 6H).

### Synthesis of 2-((S)-2,2-di((9Z,12Z)-octadeca-9,12-dien-1-yl)-1,3-dioxolan-4-yl)-N,N-dimethylethan-1-amine, (AKG-KC2-01, O-12095)

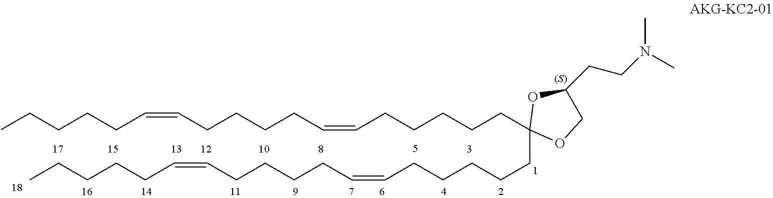

To a solution of 2-((S)-2,2-di((9Z,12Z)-octadeca-9,12-dien-1-yl)-1,3-dioxolan-4-yl) ethan-1-ol, 7 (0.49 g, 0.79 mmol) in dichloromethane (10 mL) at 0° C. was added methanesulfonyl chloride (73 L, 0.95 mmol) and triethylamine (0.26 mL, 1.2 mmol). The solution was warmed to room temperature and stirred for an addition hour. The reaction was quenched with water and extracted with dichloromethane (2×100 mL). The organics were washed with brine then dried over magnesium sulfate and filtered. The filtrate was concentrated under vacuum to give a crude oil. A solution of 2M dimethylamine (10 mL) was added to the resulting crude oil and allowed to stir for 24 hours. The mixture was then quenched with water and extracted with dichloromethane (2×100 mL). The combined organics were washed with brine then dried over magnesium sulfate then filtered. The filtrate was concentrated under vacuum to give a crude oil. The crude oil was purified by chromatography on silica using 5-100% ethyl acetate in n-hexane as eluant to give 2-((S)-2,2-di((9Z,12Z)-octadeca-9,12-dien-1-yl)-1,3-dioxolan-4-yl)-N,N-dimethylethan-1-amine, (AKG-KC2-01, O-12095), (206 mg, 0.32 mmol, 41%) as a clear oil.

¹H NMR (300 MHz, CDCl₃): 5.35-5.32 (m, 8H), 4.08-4.03 (m, 2H), 3.47 (t, J=6.8 Hz, 1H), 2.36-2.27 (m, 2H), 2.21 (s, 6H), 2.01-1.99 (m, 16H), 1.88-1.77 (m, 2H), 1.68-1.53 (m, 6H), 1.42-1.19 (m, 34H), 0.96-0.86 (t, J=3.7 Hz, 6H).

MS(APCI) for C₄₃H₇₉NO₂: 642.6

### Synthesis of 3-((S)-2,2-di((6Z, 12Z)-octadeca-6-12-dien-4-yl)-1,3-dioxolan-4-yl)-N,N-dimethylpropan-1-amine, AKG-KC3-01, O-12096)

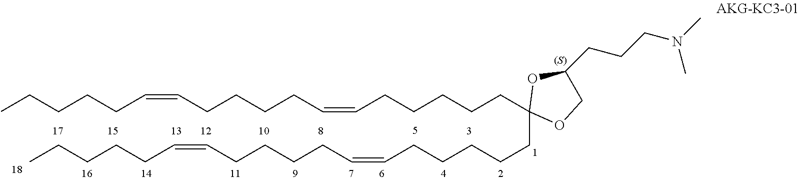

The procedure was previously described.

3-((S)-2,2-di((6Z, 12Z)-octadeca-6-12-dien-4-yl)-1,3-dioxolan-4-yl)-N,N-dimethylpropan-1-amine, (AKG-KC3-01, O-12096), (255 mg, 0.39 mmol, 51%) as a clear oil.

¹H NMR (300 MHz, CDCl₃): 5.39-5.32 (m, 8H), 4.06-4.02 (m, 2H), 3.48-3.44 (m, 1H), 2.35-2.30 (m, 2H), 2.25 (s, 6H), 2.01-1.98 (m, 16H), 1.70-1.51 (m, 12H), 1.35-1.25 (m, 32H), 0.90-0.85 (t, J=6.6 Hz, 6H).

MS(APCI) for C₄₄H₈₁NO₂: 656.6

### Synthesis of 2-((S)-2,2-di((Z)-octadec-9-en-1-yl)-1,3-dioxolan-4-yl)-N,N-dimethylethan-1-amine (AKG-KC2-OA, O-11880)

### 2-((S)-2,2-di((Z)-hexadec-9-en-1-yl)-1,3-dioxolan-4-yl)-N,N-dimethylethan-1-amine (AKG-KC2-PA, O-11879)

### 3-((S)-2,2-di((Z)-octadec-9-en-1-yl)-1,3-dioxolan-4-yl)-N,N-dimethylpropan-1-amine (AKG-KC3-OA, O-11957)

### 3-((S)-2,2-di((Z)-hexadec-9-en-1-yl)-1,3-dioxolan-4-yl)-N,N-dimethylpropan-1-amine, (AKG-KC3-PA, O-12418

FIG. 9B is a scheme showing the synthesis of AKG-KC2-OA, AKG-KC2-PA, AKG-KC3-OA, and AKG-KC3-PA

### Experimental Procedure (Refer to Previously Described Synthesis of AKG-KC2-01)

### Synthesis of (Z)-1-bromooctadec-9-ene 3

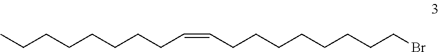 [SMILES: CCCCCCCC/C=C\CCCCCCCCBr]

The procedure was previously described.

(Z)-1-bromooctadec-9-ene, (6.4 g, 19.33 mmol) as a clear oil.

¹H NMR (300 MHz, CDCl₃): 5.36-5.32 (m, 2H), 3.41 (t, J=7.5 Hz, 2H), 2.01-1.99 (m, 4H), 1.87-1.82 (m, 2H), 1.44-1.26 (m, 22H), 0.87 (t, J=6.6 Hz, 3H).

### (Z)-16-bromohexadec-7-ene 4

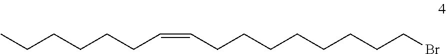 [SMILES: CCCCCC/C=C\CCCCCCCCBr]

¹H NMR (300 MHz, CDCl₃): 5.36-5.32 (m, 2H), 3.42 (t, J=7.5 Hz, 2H), 2.01-1.99 (m, 4H), 1.87-1.82 (m, 2H), 1.44-1.26 (m, 18H), 0.89 (t, J=6.6 Hz, 3H).

### Synthesis of (9Z,28Z)-heptatriaconta-9,28-dien-19-ol 5

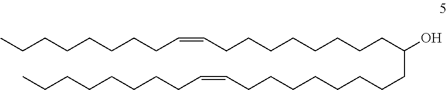 [SMILES: CCCCCCCC/C=C\CCCCCCCCC(O)CCCCCCCC/C=C\CCCCCCCC]

The procedure was previously described.

(9Z,28Z)-heptatriaconta-9,28-dien-19-ol (1.2 g, 2.25 mmol, 47%) as a solid.

¹H NMR (300 MHz, CDCl₃): 5.36-5.29 (m, 4H), 3.57 (bs, 1H), 2.01-1.97 (m, 8H), 1.42-1.26 (m, 53H), 0.89 (t, J=6.6 Hz, 6H).

### (7Z,26Z)-tritriaconta-7,26-dien-17-ol

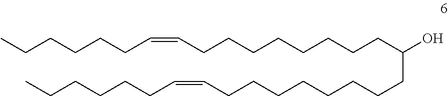 [SMILES: CCCCCC/C=C\CCCCCCCCC(O)CCCCCCCC/C=C\CCCCCC]

¹H NMR (300 MHz, CDCl₃): 5.36-5.29 (m, 4H), 3.57 (bs, 1H), 2.01-1.97 (m, 8H), 1.42-1.26 (m, 45H), 0.89 (t, J=6.6 Hz, 6H).

### Synthesis of (9Z,28Z)-heptatriaconta-9,28-dien-19-one

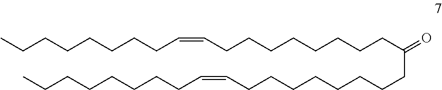 [SMILES: CCCCCCCC/C=C\CCCCCCCCC(=O)CCCCCCCC/C=C\CCCCCCCC]

The procedure was previously described.

(9Z,28Z)-heptatriaconta-9,28-dien-19-one (0.89 g, 1.67 mmol, 74%) as a clear oil.

¹H NMR (300 MHz, CDCl₃): 5.36-5.29 (m, 4H), 2.03-1.98 (m, 8H), 1.42-1.26 (m, 52H), 0.90-0.89 (t, J=6.6 Hz, 6H).

### (7Z,26Z)-tritriaconta-7,26-dien-17-one 8

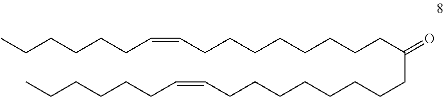 [SMILES: CCCCCC/C=C\CCCCCCCCC(=O)CCCCCCCC/C=C\CCCCCC]

¹H NMR (300 MHz, CDCl₃): 5.36-5.29 (m, 4H), 2.03-1.98 (m, 8H), 1.42-1.26 (m, 44H), 0.90-0.89 (t, J=6.6 Hz, 6H).

### Synthesis of 2-((S)-2,2-di((Z)-octadec-9-en-1-yl)-1,3-dioxolan-4-yl) ethan-1-ol 9

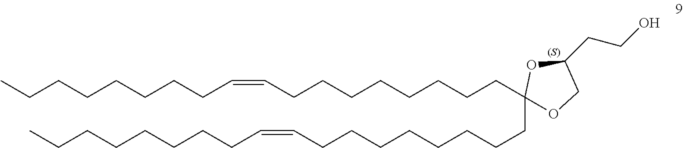

The procedure was previously described.

2-((S)-2,2-di((Z)-octadec-9-en-1-yl)-1,3-dioxolan-4-yl) ethan-1-ol (0.39 g, 0.63 mmol, 74%) as a clear oil.

¹H NMR (300 MHz, CDCl₃): 5.36-5.28 (m, 4H), 4.22-4.10 (m, 1H), 4.08-4.05 (m, 1H), 3.82-3.79 (m, 2H), 3.48 (t, J=6.8 Hz, 1H), 2.24-2.21 (m, 1H), 2.01-1.99 (m, 8H), 1.81-1.80 (m, 2H), 1.59-1.54 (m, 6H), 1.34-1.26 (m, 45H), 0.87 (t, J=6.3 Hz, 6H).

### Synthesis of 2-((S)-2,2-di((Z)-hexadec-9-en-1-yl)-1,3-dioxolan-4-yl)ethan-1-ol, 10

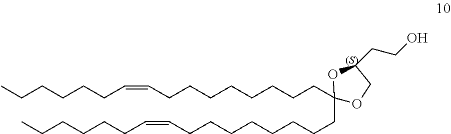

The procedure was previously described.

2-((S)-2,2-di((Z)-hexadec-9-en-1-yl)-1,3-dioxolan-4-yl)ethan-1-ol (1.02 g, 1.65 mmol, 51%) as a clear oil.

¹H NMR (300 MHz, CDCl₃): 5.36-5.29 (m, 4H), 4.23-4.10 (m, 1H), 4.07-4.05 (m, 1H), 3.82-3.79 (m, 2H), 3.48 (t, J=6.6 Hz, 1H), 2.24-2.12 (m, 1H), 2.01-1.97 (m, 8H), 1.84-1.78 (m, 2H), 1.57-1.55 (m, 8H), 1.34-1.29 (m, 35H), 0.87 (t, J=6.3 Hz, 6H).

### Synthesis of 3-((S)-2,2-di((Z)-octadec-9-en-1-yl)-1,3-dioxolan-4-yl)propan-1-ol, 11

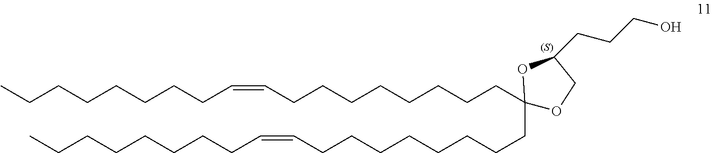

The procedure was previously described.

3-((S)-2,2-di((Z)-octadec-9-en-1-yl)-1,3-dioxolan-4-yl) propan-1-ol (0.41 g, 0.65 mmol, 76%) as a clear oil

¹H NMR (300 MHz, CDCl₃): 5.39-5.32 (m, 4H), 4.06-4.03 (m, 2H), 3.71-3.67 (m, 2H), 3.47-3.46 (m, 1H), 2.01-1.99 (m, 10H), 1.66-1.59 (m, 4H), 1.56-1.54 (m, 6H), 1.34-1.26 (m, 44H), 0.87 (t, J=6.3 Hz, 6H).

### Synthesis of 3-((S)-2,2-di((Z)-hexadec-9-en-1-yl)-1,3-dioxolan-4-yl)propan-1-ol

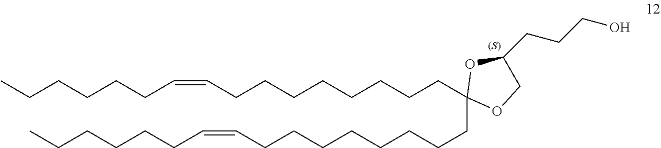

The procedure was previously described.

3-((S)-2,2-di((Z)-hexadec-9-en-1-yl)-1,3-dioxolan-4-yl)propan-1-ol, (0.9 g, 1.56 mmol, 80%) as a clear oil.

¹H NMR (300 MHz, CDCl₃): 5.39-5.28 (m, 4H), 4.06-4.01 (m, 2H), 3.71-3.67 (m, 2H), 3.47-3.46 (m, 1H), 2.01-1.99 (m, 10H), 1.66-1.59 (m, 4H), 1.56-1.54 (m, 6H), 1.34-1.26 (m, 37H), 0.87 (t, J=6.3 Hz, 6H).

### Synthesis of 2-((S)-2,2-di((Z)-octadec-9-en-1-yl)-1,3-dioxolan-4-yl)-N,N-dimethylethan-1-amine, (AKG-KC2-OA, O-11880)

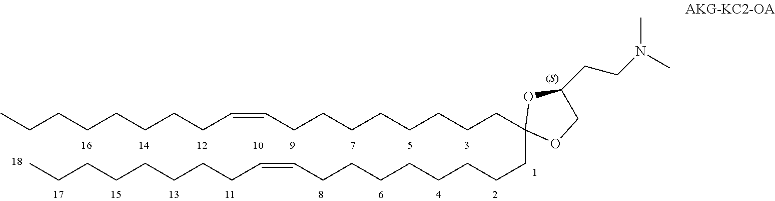

The procedure was previously described.

2-((S)-2,2-di((Z)-hexadec-9-en-1-yl)-1,3-dioxolan-4-yl)-N,N-dimethylethan-1-amine, (AKG-KC2-OA, O-11880), (200 mg, 0.31 mmol, 49%) as a clear oil.

¹H NMR (300 MHz, CDCl₃): 5.38-5.28 (m, 4H), 4.08-4.01 (m, 2H), 3.48 (t, J=6.8 Hz, 1H), 2.39-2.24 (m, 2H), 2.21 (s, 6H), 2.01-1.97 (m, 8H), 1.82-1.77 (m, 2H), 1.68-1.52 (m, 6H), 1.34-1.26 (m, 46H), 0.87 (t, J=6.3 Hz, 6H).

MS(APCI) for C₄₃H₈₃NO₂: 646.7

### Synthesis of 2-((S)-2,2-di((Z)-hexadec-9-en-1-yl)-1,3-dioxolan-4-yl)-N,N-dimethylethan-1-amine, (AKG-KC2-PA, O-11879)

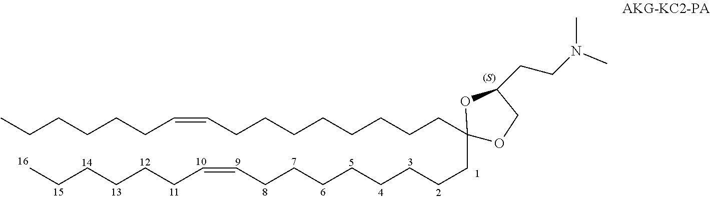

The procedure was previously described.

2-((S)-2,2-di((Z)-hexadec-9-en-1-yl)-1,3-dioxolan-4-yl)-N,N-dimethylethan-1-amine, (AKG-KC2-PA, O-11879), (195 mg, 0.33 mmol, 18%) as a clear oil.

¹H NMR (300 MHz, CDCl₃): 5.35-5.28 (m, 4H), 4.08-4.02 (m, 2H), 3.48 (t, J=6.6 Hz, 1H), 2.38-2.27 (m, 2H), 2.20 (s, 6H), 2.01-1.99 (m, 8H), 1.97-1.80 (m, 2H), 1.77-1.52 (m, 6H), 1.34-1.29 (m, 38H), 0.87 (t, J=6.3 Hz, 6H).

MS(APCI) for C₃₉H₇₅NO₂: 590.6

### Synthesis of 3-((S)-2,2-di((Z)-octadec-9-en-1-yl)-1,3-dioxolan-4-yl)-N,N-dimethylpropan-1-amine, (AKG-KC3-OA, O-11957)

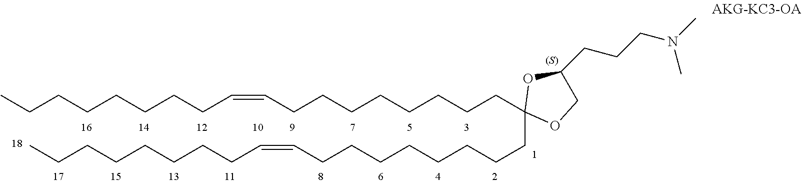

The procedure was previously described.

3-((S)-2,2-di((Z)-octadec-9-en-1-yl)-1,3-dioxolan-4-yl)-N,N-dimethylpropan-1-amine, (AKG-KC3-OA, O-11957), (160 mg, 0.24 mmol, 37%) as a clear oil.

¹H NMR (300 MHz, CDCl₃): 5.39-5.28 (m, 4H), 4.06-4.01 (m, 2H), 3.44 (t, J=6.8 Hz, 1H), 2.26 (t, J=6.8 Hz, 2H), 2.20 (s, 6H), 2.01-1.97 (m, 8H), 1.82-1.77 (m, 2H), 1.60-1.43 (m, 8H), 1.34-1.26 (m, 46H), 0.87 (t, J=6.3 Hz, 6H).

MS(APCI) for C₄₄H₈₅NO₂: 660.6

### Synthesis of 3-((S)-2,2-di((Z)-hexadec-9-en-1-yl)-1,3-dioxolan-4-yl)-N,N-dimethylpropan-1-amine, (AKG-KC3-PA, O-12418)

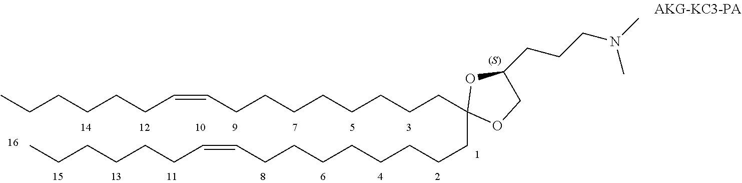

The procedure was previously described.

3-((S)-2,2-di((Z)-hexadec-9-en-1-yl)-1,3-dioxolan-4-yl)-N,N-dimethylpropan-1-amine, (AKG-KC3-PA, O-12418) (300 mg, 0.49 mmol, 32%) as a clear oil.

¹H NMR (300 MHz, CDCl₃): 5.39-5.28 (m, 4H), 4.06-4.01 (m, 2H), 3.47-3.42 (m, 1H), 2.43-2.41 (m, 2H), 2.31 (s, 6H), 2.01-1.97 (m, 8H), 1.70-1.52 (m, 6H), 1.27-1.18 (m, 42H), 0.87 (t, J=6.6 Hz, 6H).

MS(APCI) for C₄₄H₈₅NO₂: 604.6

### Synthesis of 3-((S)-2,2-di((Z)-heptadec-8-en-1-yl)-1,3-dioxolan-4-yl)-N,N-dimethylpropan-1-amine, AKG-KC3-C17(C8:1)

### Synthesis of (S)-3-(2,2-diheptadecyl-1,3-dioxolan-4-yl)-N,N-dimethylpropan-1-amine, AKG-KC3-C17

FIG. 9C is a scheme showing the synthesis of AKG-KC3-C17(C8:1) and AKG-KC3-C17.

### Experimental Procedure

### Synthesis of (9Z,26Z)-pentatriaconta-9,26-dien-18-one, 2

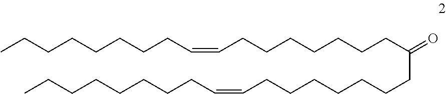 [SMILES: CCCCCCCC/C=C\CCCCCCCC(=O)CCCCCCC/C=C\CCCCCCCC]

To a stirring solution of oleoyl chloride (10 g, 33.3 mmol) in toluene (50 mL) at 0° C. was added triethylamine (5.8 mL, 33.3 mmol). A heavy precipitate formed, and the mixture was allowed to stir at room temperature for 8 hours. The mixture was quenched with 2% sulfuric acid solution and then extracted with ethyl acetate. The organics were washed with brine then dried over magnesium sulfate and filtered. The filtrate was concentrated under vacuum to give a crude oil. The resulting oil was diluted with ethanol (20 mL) and [2N NaOH](30 mL) was added. The mixture was heated at 100° C. for 12 hours then cooled. The mixture was diluted with 2N HCl solution until a pH 4 was obtained. The mixture was extracted with ethyl acetate. The combined organics were washed with brine then dried over magnesium sulfate and filtered. The filtrate was concentrated under vacuum to give a crude oil. Purification of the oil on silica using 10-20% ethyl acetate in n-hexane as eluant gave (9Z,26Z)-pentatriaconta-9,26-dien-18-one, 2 (3.8 g, 44%) as a yellow oil.

¹H NMR (300 MHz, CDCl₃): δ ppm 5.35-5.31 (m, 4H), 2.39-2.34 (m, 4H), 2.0-1.85 (m, 8H), 1.57-1.52 (m, 4H), 1.27-1.25 (m, 40H), 0.88 (t, J=6.6 Hz, 3H).

### Synthesis of 3-((S)-2,2-di((Z)-heptadec-8-en-1-yl)-1,3-dioxolan-4-yl)propan-1-ol, 3

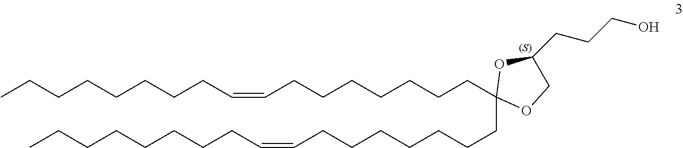

Procedure previously described synthesis of AKG-KC2-01

3-((S)-2,2-di((Z)-heptadec-8-en-1-yl)-1,3-dioxolan-4-yl) propan-1-ol, 3 (0.7 g, 1.15 mmol, 65%) as a clear oil.

¹H NMR (300 MHz, CDCl₃): δ ppm 5.38-5.31 (m, 4H), 4.08-4.02 (m, 2H), 3.67-3.66 (m, 2H), 3.48-3.43 (m, 1H), 2.15-2.13 (m, 1H), 2.00-1.98 (m, 8H), 1.65-1.56 (m, 8H), 1.27-1.25 (m, 44H), 0.88 (t, J=6.6 Hz, 6H).

### Synthesis of (S)-3-(2,2-diheptadecyl-1,3-dioxolan-4-yl)propan-1-ol, 4

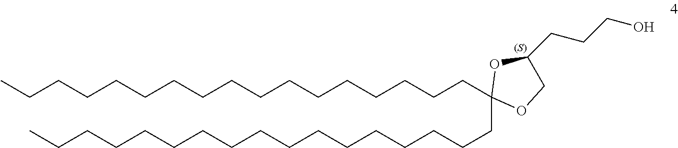

A solution of 3-((S)-2,2-di((Z)-heptadec-8-en-1-yl)-1,3-dioxolan-4-yl) propan-1-ol (1.3 g, 2.15 mmol) in methanol/ethyl acetate (20 mL, 1:1/v:v) was hydrogenated at 1 atm (hydrogen balloon) over 10% palladium on carbon (100 mg) for 2 hours. The mixture was evacuated of hydrogen and flowed with nitrogen. The mixture was filtered over celite, and the filtrate concentrated under vacuum to give (S)-3-(2,2-diheptadecyl-1,3-dioxolan-4-yl) propan-1-ol, 4 (1.3 g, quant.) as a clear oil.

¹H NMR (300 MHz, CDCl₃): δ ppm 4.10-4.01 (m, 2H), 3.71-3.64 (m, 1H), 3.47-3.43 (m, 2H), 2.05-2.01 (m, 1H), 1.63-1.51 (m, 8H), 1.42-1.22 (m, 58H), 0.87 (t, J=6.6 Hz, 6H).

### Synthesis of 3-((S)-2,2-di((Z)-heptadec-8-en-1-yl)-1,3-dioxolan-4-yl)-N,N-dimethylpropan-1-amine, AKG-KC3-C17(C8:1)(O-12620)

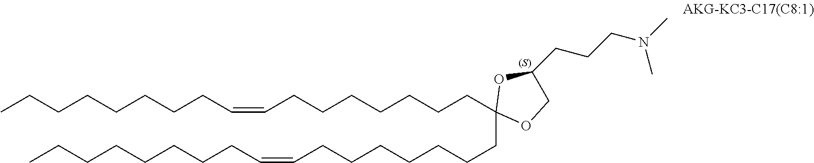

Procedure previously described synthesis of AKG-KC2-01

3-((S)-2,2-di((Z)-heptadec-8-en-1-yl)-1,3-dioxolan-4-yl)-N, N-dimethylpropan-1-amine, (AKG-KC3-C17 (C8:1), O-12620), (290 mg, 0.46 mmol, 40%) a clear oil.

MS (APCI+) for C₄₂H₈₁NO₂: 632.6

¹H NMR (300 MHz, CDCl₃): δ ppm 5.38-5.28 (m, 4H), 4.09-3.99 (m, 2H), 3.48-3.41 (m, 1H), 2.77-2.71 (m, 1H), 2.55 (s, 6H), 2.01-1.95 (m, 8H), 1.88-1.78 (m, 2H), 1.62-1.50 (m, 6H), 1.27-1.24 (m, 44H), 0.88 (t, J=6.8 Hz, 6H).

### Synthesis of (S)-3-(2,2-diheptadecyl-1,3-dioxolan-4-yl)-N,N-dimethylpropan-1-amine, AKG-KC3-C17 (O-12637)

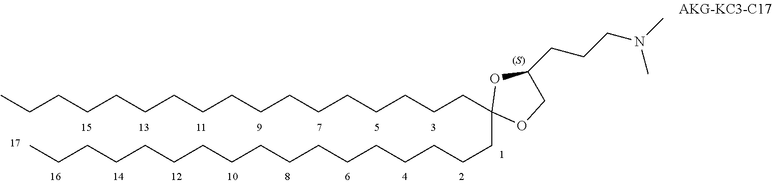

Procedure previously described synthesis of AKG-KC2-01

(S)-3-(2,2-diheptadecyl-1,3-dioxolan-4-yl)-N, N-dimethylpropan-1-amine, (AKG-KC3-C17, O-12637), (275 mg, 0.43 mmol, 22%) as a solid.

MS (APCI+) for C₄₂H₈₅NO₂: 636.6

¹H NMR (300 MHz, CDCl₃): δ ppm 4.06-4.00 (m, 2H), 3.47-3.43 (m, 1H), 2.29-2.25 (m, 2H), 2.21 (s, 6H), 1.60-1.48 (m, 8H), 1.29-1.24 (m, 60H), 0.87 (t, J=6.6 Hz, 6H).

### Synthesis of (S)-4-(dimethylamino)butane-1,2-diyl dioleate (AKG-OA-DM2, O-12137)

### Synthesis of (S)-5-(dimethylamino)pentane-1,2-diyl dioleate (AKG-OA-DM3, O-12138)

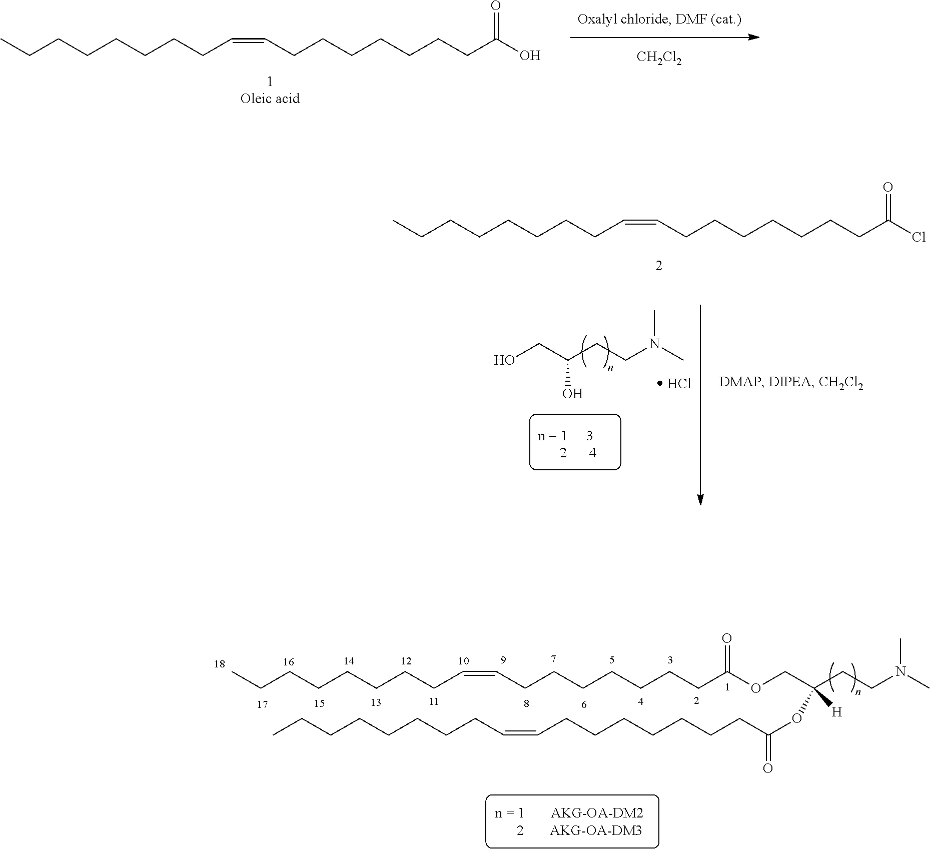

### Synthesis of (S)-4-(dimethylamino)butane-1,2-diyl dioleate (AKG-OA-DM2, O-12137)

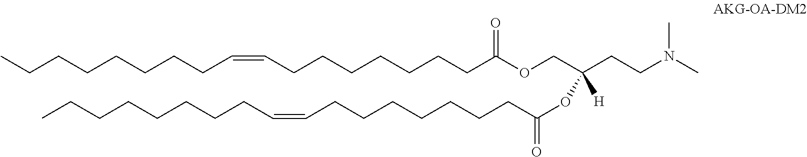 [SMILES: CCCCCCCC/C=C\CCCCCCCC(=O)OC[C@H](CCN(C)C)OC(=O)CCCCCCC/C=C\CCCCCCCC]

Oxalyl chloride (1.8 mL, 21.3 mmol) was added dropwise to a solution of oleic acid, 14 (2 g, 7.1 mmol) in dichloromethane/DMF (40 mL, 25 mL) at 0° C. and allowed reaction to warm to room temperature and stir for one hour. After one hour, the reaction was concentrated under vacuum to dryness. The acid chloride was dissolved in dichloromethane and added to a solution of (S)-4-(diethylamino)butane-1,2-diol hydrochloride salt (0.54 g, 3.22 mmol), 4-dimethylaminopyridine (0.78 g, 6.44 mmol), and N,N-diisopropylethylamine (5.2 mL, 32.2 mmol). The resulting mixture was stirred at room temperature for 20 hours then quenched with water. The mixture was extracted with dichloromethane (2×50 mL). The combined organics were washed with water then dried over magnesium sulfate, filtered and the filtrate concentrated under vacuum to give a crude oil. The crude oil was purified by chromatography on silica using 2% methanol in chloroform as eluant to give (S)-4-(dimethylamino)butane-1,2-diyl dioleate (820 mg, 38%) as a clear oil.

¹H NMR (300 MHz, CDCl₃): 5.35-5.31 (m, 4H), 5.13-5.07 (m, 1H), 4.26 (dd, J=11.8, 3.3 Hz, 1H), 4.08-4.02 (m, 1H), 2.36-2.26 (m, 6H), 2.20 (s, 6H), 2.00-1.96 (m, 8H), 1.79-1.74 (m, 2H), 1.60-1.59 (m. 4H), 1.28-1.25 (m, 40H), 0.92-0.85 (m, 6H).

MS (APCI+): 662.6 (M+1)

(S)-5-(dimethylamino)pentane-1,2-diyl dioleate (AKG-OA-DM3, O-12138)

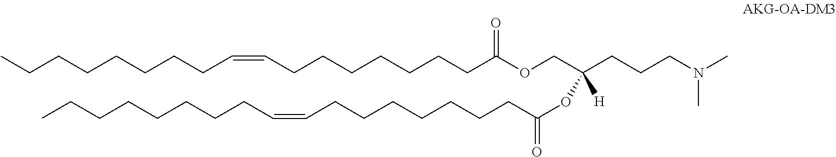 [SMILES: CCCCCCCC/C=C\CCCCCCCC(=O)OC[C@H](CCCN(C)C)OC(=O)CCCCCCC/C=C\CCCCCCCC]

Procedure as previously described.

¹H NMR (300 MHz, CDCl₃): 5.35-5.31 (m, 4H), 5.10-5.05 (m, 1H), 4.24 (dd, J=11.8, 3.3 Hz, 1H), 4.05-3.99 (m, 1H), 2.32-2.26 (m, 6H), 2.20 (s, 6H), 2.00-1.97 (m, 10H), 1.79-1.74 (m, 2H), 1.60-1.59 (m. 4H), 1.28-1.25 (m, 40H), 0.89-0.85 (m, 6H).

MS (APCI⁺): 676.6 (M+1)

### Example 1B. Synthesis of Ionizable Lipids

FIG. 9D is a reaction scheme for the preparation of a KC3-X1 ionizable lipid.

### Synthesis of KC3-X1-PH5 Ionizable Lipid

### Procedure for Preparation of Compound 2

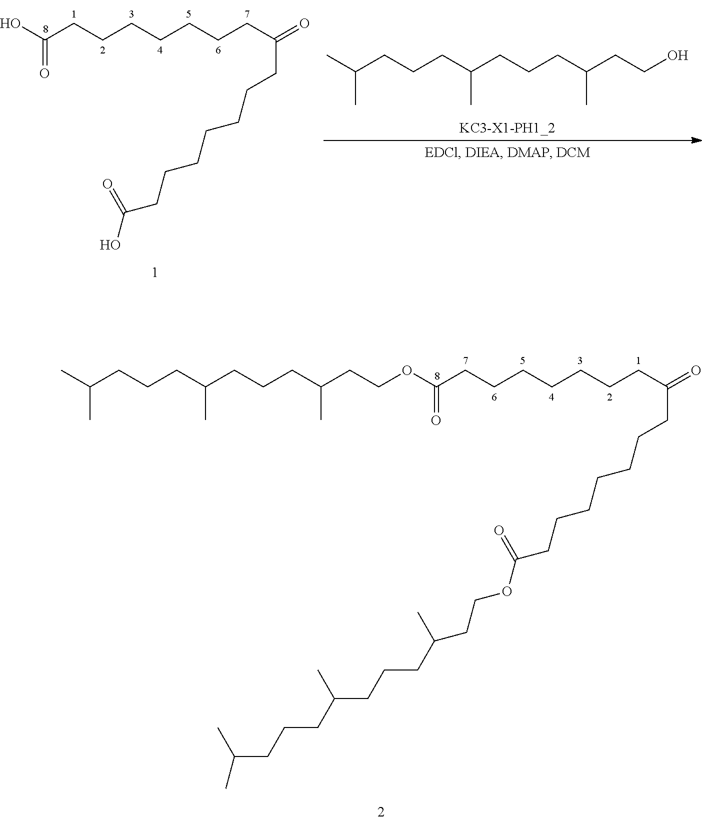

To a solution of compound 1 (3 g, 9.54 mmol, 1 eq) and KC3-X1-PH1_2 (4.36 g, 19.08 mmol, 2 eq) in DCM (30 mL) was added EDCI (7.32 g, 38.17 mmol, 4 eq) and DMAP (2.91 g, 23.85 mmol, 2.5 eq) and DIEA (12.33 g, 95.42 mmol, 16.62 mL, 10 eq). The mixture was stirred at 40° C. for 12 hr. Upon completion, the mixture was concentrated in vacuo to give a residue. The residue was purified by flash silica gel chromatography (ISCO®; 80 g SepaFlash® Silica Flash Column, Eluent of 0-4% Ethyl acetate/Petroleum ether gradient @100 mL/min) to give compound 2 (6.8 g, 9.24 mmol, 96.8% yield, 99.9% purity) as a yellow solid.

¹H NMR (400 MHz, CHLOROFORM-d) δ 4.08-3.97 (m, 4H), 2.30 (t, J=7.2 Hz, 4H), 2.21 (t, J=7.6 Hz, 4H), 1.61-1.42 (m, 15H), 1.39-1.27 (m, 5H), 1.25-0.96 (m, 34H), 0.84-0.75 (m, 24H).

### Procedure for Preparation of Compound 4

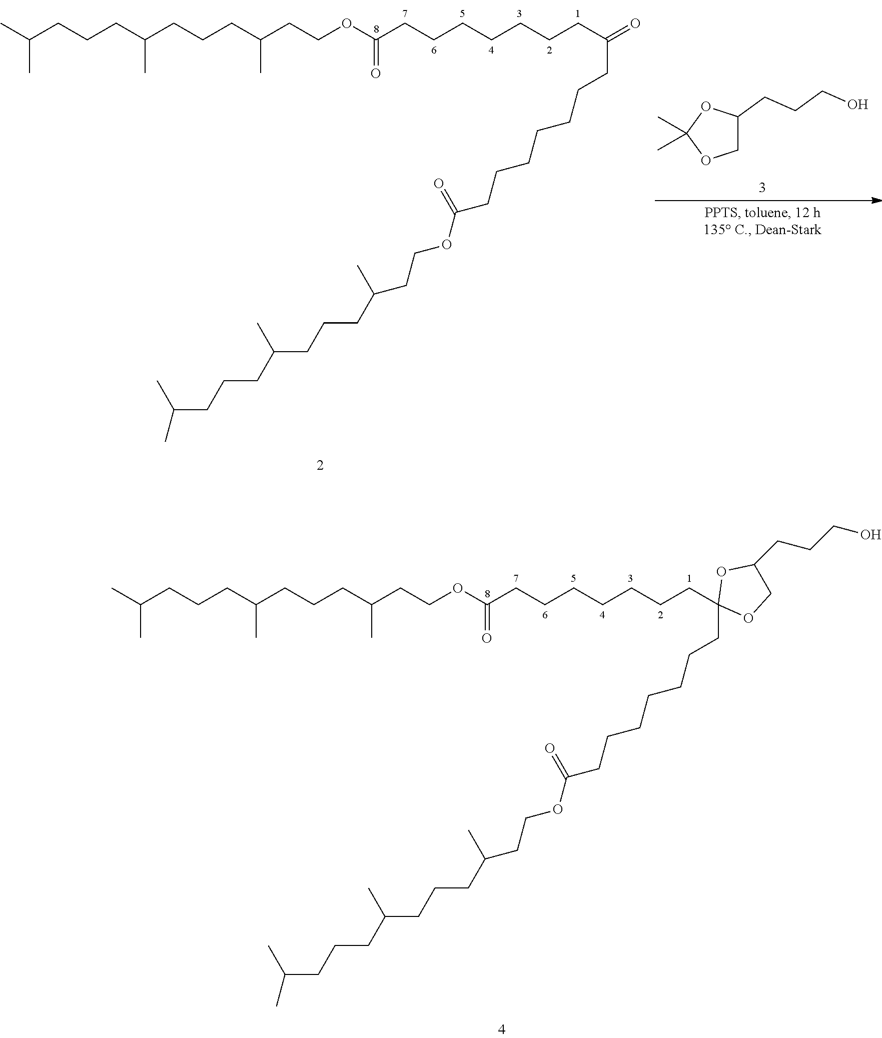 [SMILES: CC(C)CCCC(C)CCCC(C)CCOC(=O)CCCCCCCC(=O)CCCCCCCC(=O)OCCC(C)CCCC(C)CCCC(C)C ; CC1(C)OCC(CCCO)O1 ; CC(C)CCCC(C)CCCC(C)CCOC(=O)CCCCCCCC1(CCCCCCCC(=O)OCCC(C)CCCC(C)CCCC(C)C)OCC(CCCO)O1]

To a mixture of compound 2 (2 g, 2.72 mmol, 1 eq) and compound 3 (1.09 g, 6.80 mmol, 2.5 eq) in Tol. (20 mL) was added PPTS (341.81 mg, 1.36 mmol, 0.5 eq), and the mixture was heated to reflux and remove water by Dean-Stark trap for 12 hr. The reaction solution was added into saturated NaHCO₃ (30 mL) solution, and the aqueous phase was extracted three times with DCM (20 mL*3). The combined organic layers were washed with brine (20 mL), dried over Na₂SO₄, filtered and concentrated in vacuo. The residue was purified by flash silica gel chromatography (ISCO®; 40 g SepaFlash® Silica Flash Column, Eluent of 0˜20% Ethyl acetate/Petroleum ether gradient @60 mL/min) to give compound 4 (1.8 g, 2.15 mmol, 79.1% yield) as a yellow oil.

1H NMR (400 MHz, CHLOROFORM-d, EW58311-220-P1A1) δ 4.18-4.07 (m, 6H), 3.72 (qd, J=5.2, 10.2 Hz, 2H), 3.54-3.45 (m, 1H), 2.32 (t, J=7.6 Hz, 4H), 1.72-1.52 (m, 18H), 1.50-1.22 (m, 34H), 1.21-1.04 (m, 10H), 1.01-0.78 (m, 24H).

### Procedure for Preparation of Compound 5

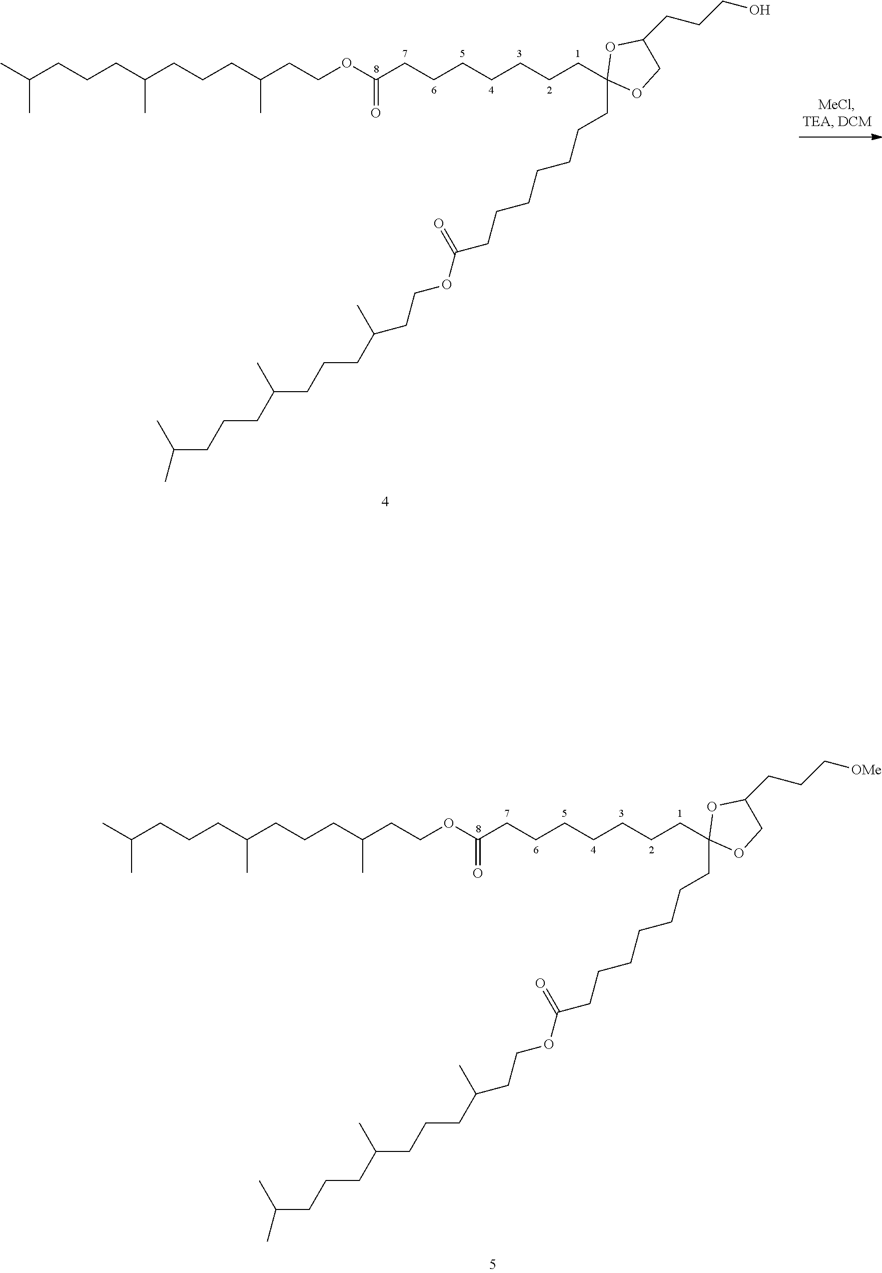 [SMILES: CC(C)CCCC(C)CCCC(C)CCOC(=O)CCCCCCCC1(CCCCCCCC(=O)OCCC(C)CCCC(C)CCCC(C)C)OCC(CCCO)O1 ; COCCCC1COC(CCCCCCCC(=O)OCCC(C)CCCC(C)CCCC(C)C)(CCCCCCCC(=O)OCCC(C)CCCC(C)CCCC(C)C)O1]

To a solution of compound 4 (1.8 g, 2.15 mmol, 1 eq) in DCM (20 mL) was added TEA (1.09 g, 10.75 mmol, 1.50 mL, 5 eq) and MsCl (410 mg, 3.58 mmol, 277.03 μL, 1.67 eq). The mixture was stirred at 0° C. for 1 hr. The residue was poured into ice water (20 mL) and stirred for 2 min. The aqueous phase was extracted with DCM (20 mL*3). The combined organic phases were washed with brine (10 mL*3), dried with anhydrous Na₂SO₄, filtered and concentrated in vacuum. The residue was purified by flash silica gel chromatography (ISCO®; 40 g SepaFlash® Silica Flash Column, Eluent of 0-15% Ethyl acetate/Petroleum ether gradient @60 mL/min) to give compound 5 (1.6 g, 1.75 mmol, 81.3% yield) was as a yellow oil.

¹H NMR (400 MHz, CHLOROFORM-d, EW58311-223-P1A1) δ 4.35-4.25 (m, 2H), 4.15-4.04 (m, 6H), 3.52-3.45 (m, 1H), 3.04 (s, 3H), 2.30 (t, J=7.6 Hz, 4H), 2.00-1.79 (m, 2H), 1.71-1.52 (m, 17H), 1.50-1.23 (m, 34H), 1.18-1.04 (m, 10H), 0.94-0.85 (m, 24H).

### Procedure for Preparation of Compound KC3-X1-PH5

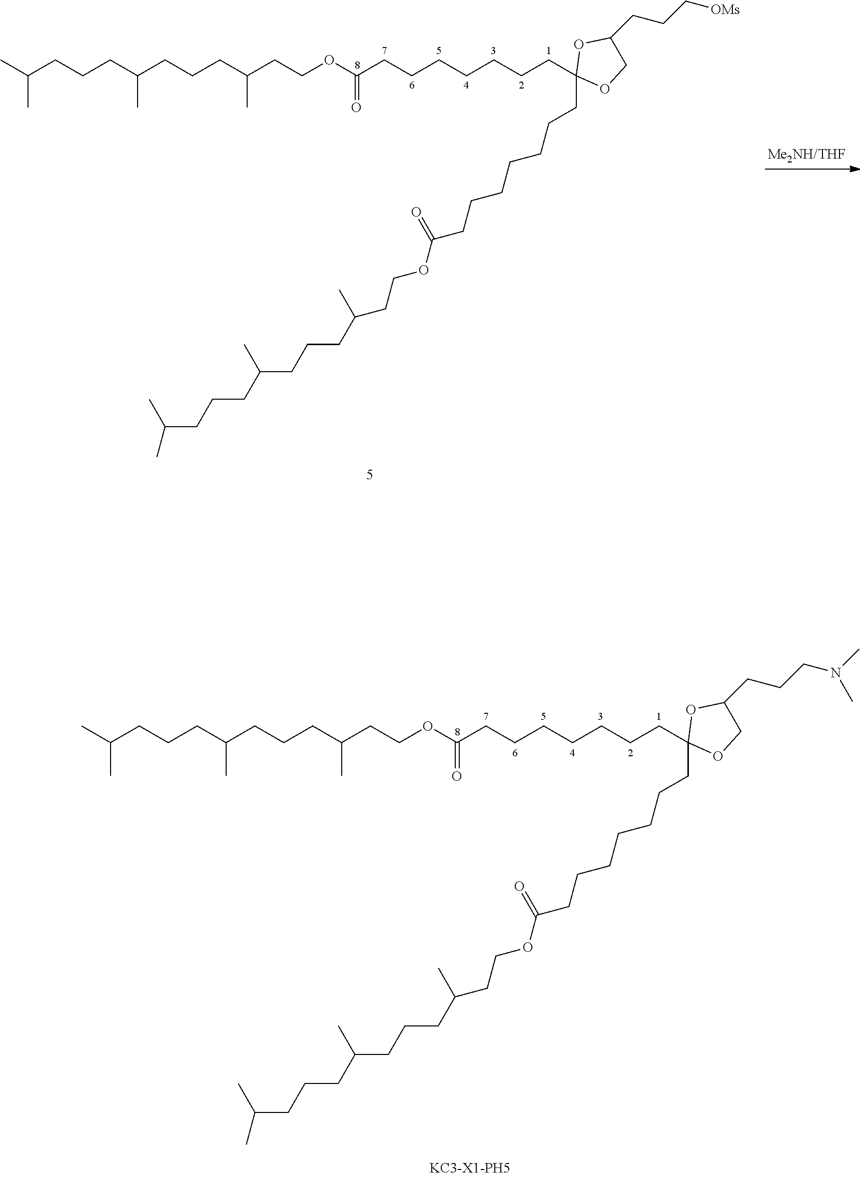 [SMILES: CC(C)CCCC(C)CCCC(C)CCOC(=O)CCCCCCCC1(CCCCCCCC(=O)OCCC(C)CCCC(C)CCCC(C)C)OCC(CCCOS(C)(=O)=O)O1 ; CC(C)CCCC(C)CCCC(C)CCOC(=O)CCCCCCCC1(CCCCCCCC(=O)OCCC(C)CCCC(C)CCCC(C)C)OCC(CCCN(C)C)O1]

A solution of compound 5 (1.5 g, 1.64 mmol, 1 eq) in Me₂NH/THF (2 M, 15 mL) was stirred at 55° C. for 12 hr. The mixture was concentrated in vacuo to give a residue. The residue was purified by flash silica gel chromatography (ISCO®; 20 g SepaFlash® Silica Flash Column, Eluent of 0-8% Ethyl acetate/(Petroleum ether/Dichloromethane=3/1, 0.1% TEA as eluant) @40 mL/min) to give KC3-X1-PH5 (553.7 mg, 624.54 mol, 38.11% yield, 97.5% purity) as a yellow oil.

LCMS (EW58311-226-P1C₁): CAD, m/z, M+H=865.0.

HRMS (ESI) m/z: [M+H]⁺ Calcd for C₅₄H₁₀₆NO₆⁺ 864.8015, found 864.8044.

¹H NMR (400 MHz, CHLOROFORM-d, EW58311-226-P1A1) δ 4.20-3.98 (m, 6H), 3.52-3.41 (m, 1H), 2.35-2.17 (m, 12H), 1.75-1.49 (m, 18H), 1.48-1.20 (m, 34H), 1.19-0.98 (m, 10H), 0.95-0.83 (m, 24H).

### Example 1C. Synthesis of Ionizable Lipids

FIG. 9E is a reaction scheme for the preparation of a KC3-X2 ionizable lipid.

### Synthesis of KC3-X2 Ionizable Lipids

The KC3-X2 ionizable lipid was prepared according to the following synthetic scheme, as further detailed below. FIG. 9E is a reaction scheme for the preparation of a KC3-X2 ionizable lipid. The KC3-X2 ionizable lipid was prepared according to the following synthetic scheme, as further detailed in FIG. 9E.

### Step 1: Preparation of Intermediate Compound 2.

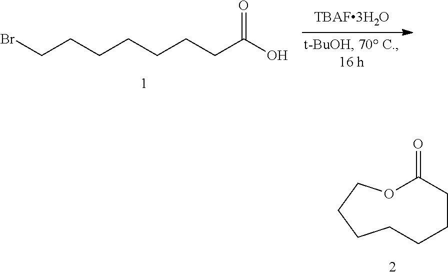 [SMILES: O=C(O)CCCCCCCBr ; O=C1CCCCCCCO1]

To a solution of compound 1 (50 g, 224.11 mmol, 1 eq) in t-BuOH (500 mL) was added TBAF·3H₂O (141.42 g, 448.22 mmol, 2 eq). The mixture was stirred at 70° C. for 16 hrs. The reaction was diluted with water (500 mL) and EtOAc (500 mL). The organic layer was washed with water (500 mL*2), dried over anhydrous Na₂SO₄, filtered and concentrated under reduced pressure. The residue was purified by column chromatography (SiO₂, Petroleum ether/Ethyl acetate=10/1 to 5/1) to give compound 2 (7.5 g, 52.74 mmol, 12% yield) as a white solid.

¹H NMR (CHLOROFORM-d, 400 MHz): δ 4.09-4.16 (m, 2H), 2.33 (t, J=6.8 Hz, 2H), 1.59-1.71 (m, 4H), 1.27-1.44 (m, 6H)

### Step 2: Preparation of Intermediate Compound 3.

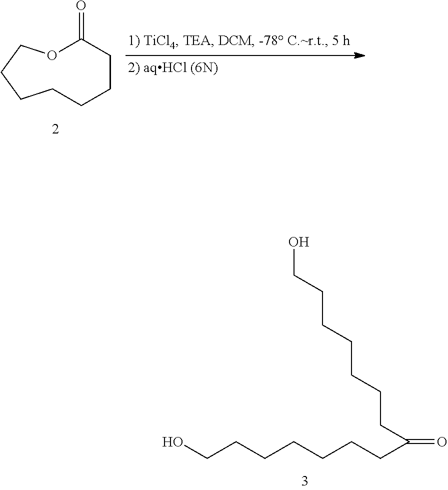 [SMILES: O=C1CCCCCCCO1 ; O=C(CCCCCCCO)CCCCCCCO]

To a solution of compound 2 (3.6 g, 25.32 mmol, 2 eq) in DCM (50 mL) was added TEA (4.48 g, 44.31 mmol, 6.17 mL, 3.5 eq) and TiCl₄ (4.80 g, 25.32 mmol, 2 eq). The mixture was stirred at −78° C. for 4 hrs. The reaction solution was added into ice water (60 mL) and extracted with the mixture of DCM/MeOH=10:1 (50 mL*3). The combined organic layers were washed with brine (50 mL), dried over Na₂SO₄, and filtered concentrated under reduced pressure to give a residue. Then HCl (6N, 1.58 mL, 1 eq) was added and the mixture was stirred at 80° C. for 4 hr, then the mixture was extracted with the solution of DCM/MeOH=10:1 (50 mL*3), the organic layer was concentrated under reduced pressure, the residue was purified by column chromatography (SiO2, DCM: MeOH=100:1 to 10:1) to give Compound 3 (1.8 g, 6.97 mmol, 28% yield) as a white solid.

¹H NMR (CHLOROFORM-d, 400 MHz): δ 3.64 (t, J=6.4 Hz, 4H), 2.39 (t, J=7.6 Hz, 4H), 1.53-1.62 (m, 8H), 1.28-1.38 (m, 12H)

### Step 3: Preparation of Intermediate Compound 5.

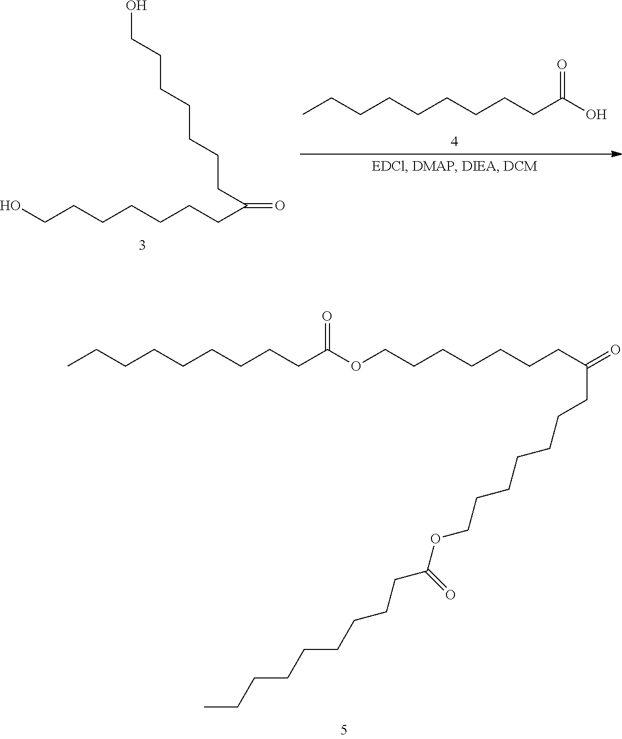 [SMILES: O=C(CCCCCCCO)CCCCCCCO ; CCCCCCCCCC(=O)O ; CCCCCCCCCC(=O)OCCCCCCCC(=O)CCCCCCCOC(=O)CCCCCCCCC]

To a solution of compound 3 (1 g, 3.87 mmol, 1 eq) and compound 4 (2.00 g, 11.61 mmol, 2.24 mL, 3 eq) in DCM (20 mL) was added DIEA (3.00 g, 23.22 mmol, 4.04 mL, 6 eq), EDCI (2.23 g, 11.61 mmol, 3 eq) and DMAP (945.58 mg, 7.74 mmol, 2 eq). The mixture was stirred at 30° C. for 12 hrs. The reaction mixture was concentrated under reduced pressure to remove solvent. The residue was purified by column chromatography (SiO2, Petroleum ether/Ethyl acetate=20/1 to 10/1) to give compound 5 (2.01 g, 3.55 mmol, 92% yield) was obtained as a white solid.

¹H NMR (CHLOROFORM-d, 400 MHz): δ 4.05 (t, J=6.8 Hz, 4H), 2.39 (t, J=7.6 Hz, 4H), 2.29 (t, J=7.6 Hz, 4H), 1.61-1.68 (m, 5H), 1.52-1.59 (m, 5H), 1.19-1.39 (m, 37H), 0.85-0.92 (m, 6H)

### Step 4: Preparation of Intermediate Compound 6.

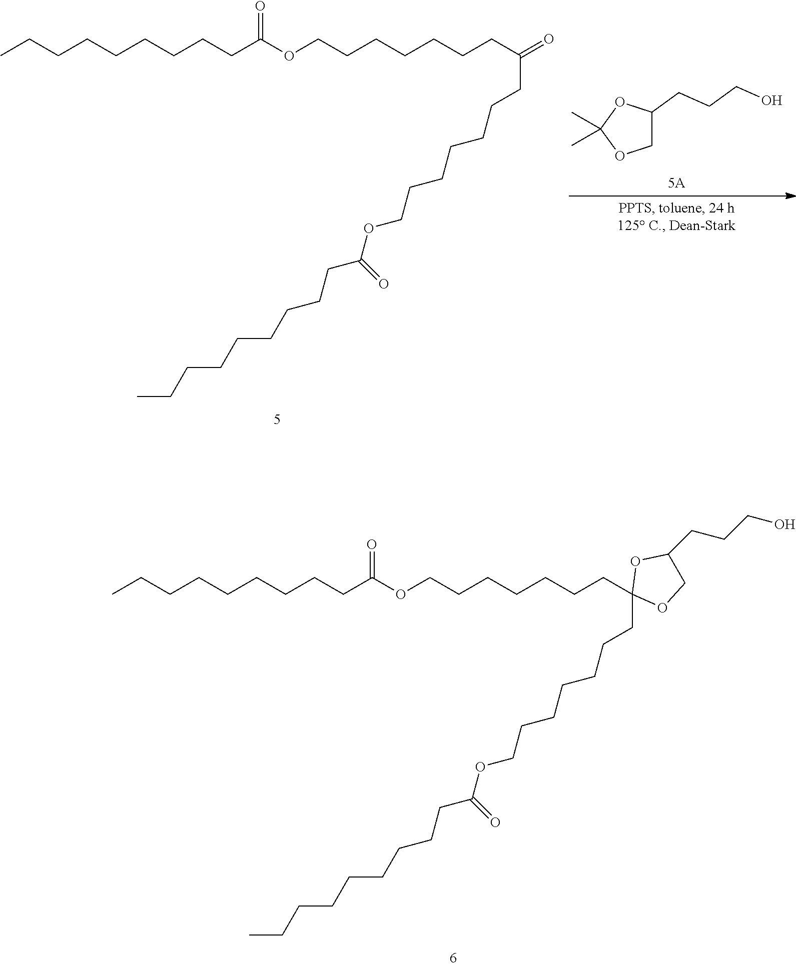 [SMILES: CCCCCCCCCC(=O)OCCCCCCCC(=O)CCCCCCCOC(=O)CCCCCCCCC ; CC1(C)OCC(CCCO)O1 ; CCCCCCCCCC(=O)OCCCCCCCC1(CCCCCCCOC(=O)CCCCCCCCC)OCC(CCCO)O1]

To a mixture of compound 5 (2 g, 3.53 mmol, 1 eq), compound 5A (1.70 g, 10.58 mmol, 3 eq) in Tol. (30 mL) was added PPTS (443.30 mg, 1.76 mmol, 0.5 eq). The mixture was heated to reflux with the Dean-Stark trap. The mixture was stirred at 125° C. for 12 hrs. The reaction solution was treated with saturated NaHCO₃ solution (20 mL) and the aqueous phase was extracted three times with DCM (50 mL*3), dried over Na₂SO₄, filtered and concentrated under reduced pressure. The residue was purified by column chromatography (SiO₂, Petroleum ether/Ethyl acetate=20/1 to 10/1) to give Compound 6 (1.02 g, 1.52 mmol, 43% yield) as a colorless oil.

¹H NMR (CHLOROFORM-d, 400 MHz): δ 4.03-4.09 (m, 6H), 3.69 (br d, J=4.4 Hz, 2H), 3.42-3.53 (m, 1H), 2.29 (t, J=7.6 Hz, 4H), 2.08-2.21 (m, 1H), 1.53-1.73 (m, 17H), 1.19-1.40 (m, 41H), 0.83-0.94 (m, 6H)

### Step 5: Preparation of Intermediate Compound 7.

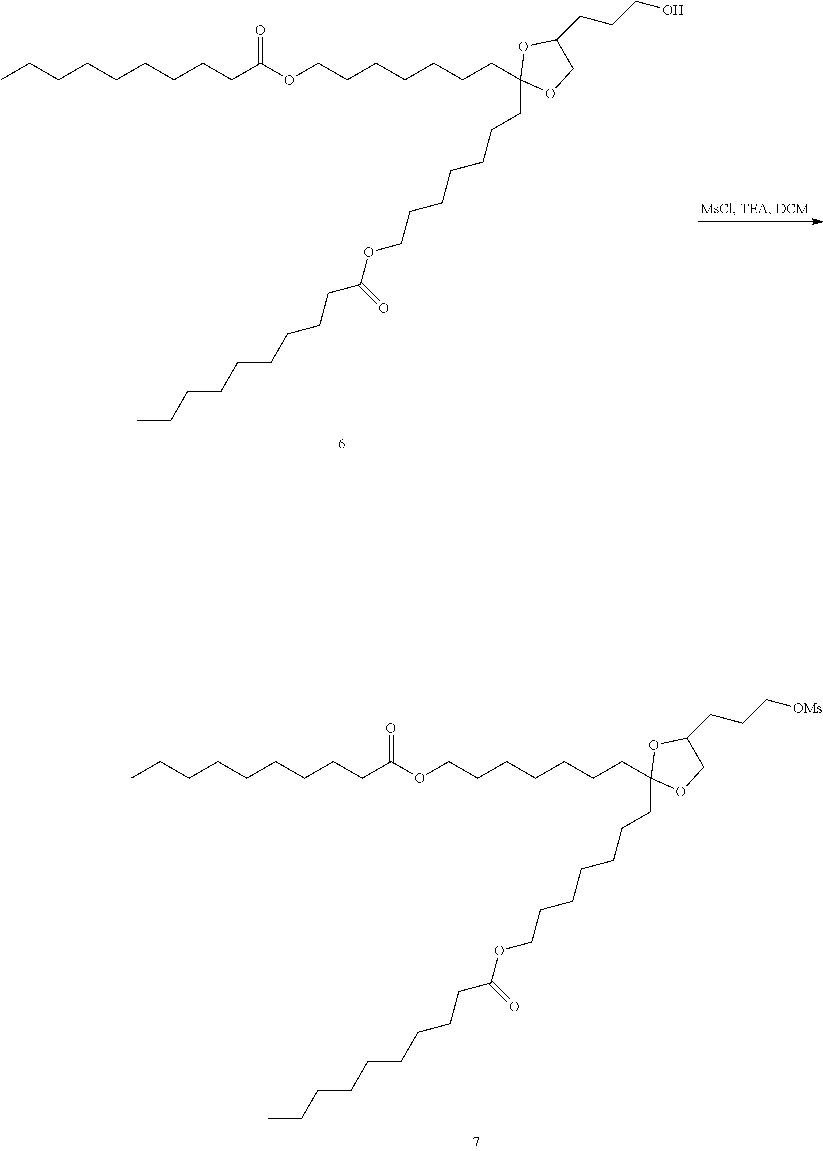 [SMILES: CCCCCCCCCC(=O)OCCCCCCCC1(CCCCCCCOC(=O)CCCCCCCCC)OCC(CCCO)O1 ; CCCCCCCCCC(=O)OCCCCCCCC1(CCCCCCCOC(=O)CCCCCCCCC)OCC(CCCOS(C)(=O)=O)O1]

To a solution of compound 6 (1.02 g, 1.52 mmol, 1 eq) in DCM (10 mL) was added TEA (1.09 g, 10.78 mmol, 1.5 mL, 7.07 eq) and then slowly added MsCl (643 mg, 5.61 mmol, 434.46 μL, 3.68 eq) at 0° C. The mixture was stirred at 25° C. for 2 hrs. The reaction mixture was diluted with water (10 mL) and extracted with DCM (10 mL*3). The organic layer was dried over Na₂SO₄, filtered and concentrated to give the crude product. The residue was purified by column chromatography (SiO2, Petroleum ether/Ethyl acetate=10/1 to 3/1) to give compound 7 (1.22 g, 1.50 mmol, 99% yield, 92% purity) as a colorless oil.

¹H NMR (CHLOROFORM-d, 400 MHz): δ 4.19-4.36 (m, 2H), 3.99-4.13 (m, 6H), 3.41-3.50 (m, 1H), 3.01 (s, 3H), 2.28 (t, J=7.6 Hz, 4H), 1.73-1.99 (m, 2H), 1.51-1.65 (m, 12H), 1.17-1.42 (m, 38H), 0.80-0.95 (m, 6H)

Step 6: Preparation of KC3-X2 Ionizable Lipid from Intermediate Compound 7.

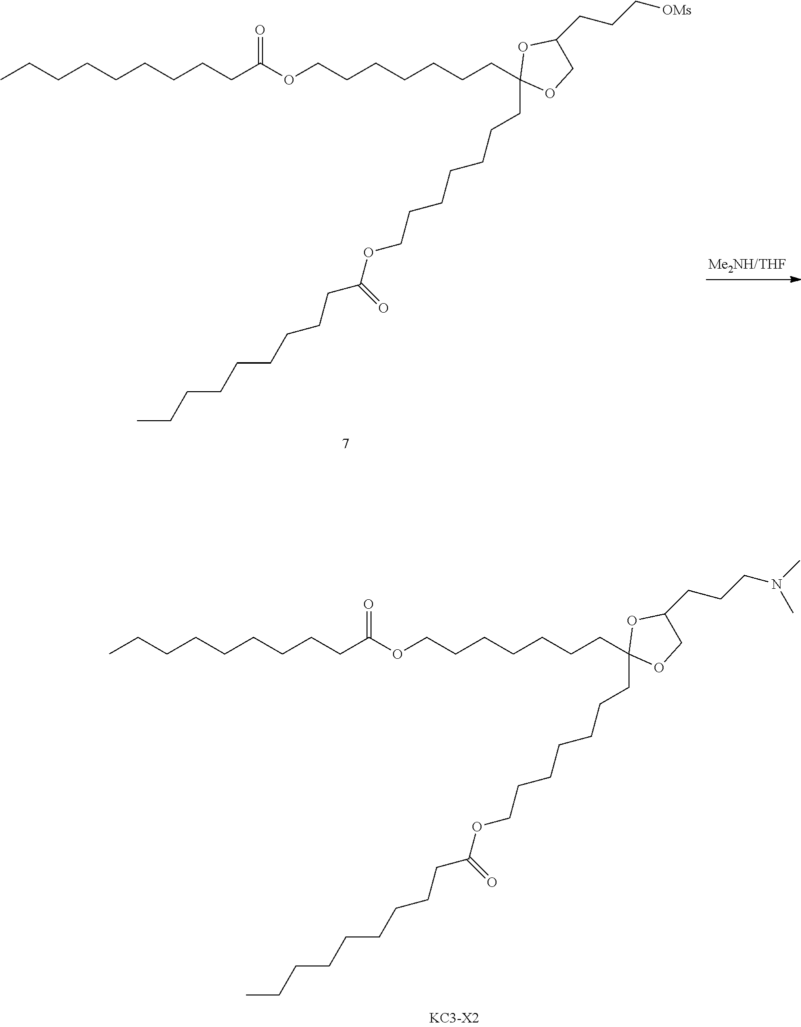 [SMILES: CCCCCCCCCC(=O)OCCCCCCCC1(CCCCCCCOC(=O)CCCCCCCCC)OCC(CCCOS(C)(=O)=O)O1 ; CCCCCCCCCC(=O)OCCCCCCCC1(CCCCCCCOC(=O)CCCCCCCCC)OCC(CCCN(C)C)O1]

A solution of compound 7 (1.22 g, 1.63 mmol, 1 eq) in Me₂NH/THF (2 M, 30 mL) was stirred at 55° C. for 12 hr. The reaction mixture was concentrated under reduced pressure to remove solvent. The residue was purified by column chromatography (SiO2, Petroleum ether/Ethyl acetate=50/1 to 5/1, 0.1% TEA as eluant) to give KC3-X2 (300 mg, 413.74 mol, 48% yield, 96% purity) as a yellow oil.

¹H NMR (CHLOROFORM-d, 400 MHz): δ (ppm) 4.06 (t, J=6.8 Hz, 6H), 3.41-3.50 (m, 1H), 20.18-2.35 (m, 12H), 1.53-1.70 (m, 17H), 1.22-1.39 (m, 41H), 0.83-0.94 (m, 6H)

LCMS: ESI+, m/z, [M+H]⁺=696.7

### Example 1D: Synthesis of Cationic Lipids with Asymmetric Chains and Also where One Chain is Saturated Alkyl and Another is Monounsaturated, C15 or C17

### Synthesis of (7Z, 24Z)-tritriaconta-7,24-dien-16-one (asymmetric C₁₅(8:1)-C₁₇(8:1) ketone)

An equimolar mixture of oleoyl chloride and palmitoleoyl chloride is processed essentially as described above for the synthesis of (9Z,26Z)-pentatriaconta-9,26-dien-18-one (2). The products (C₁₅(8:1)-C₁₅(8:1), C₁₇(8:1)-C₁₇(8:1), and C₁₅(8:1)-C₁₇(8:1) ketones) are separated using column chromatography to isolate the asymmetric C₁₅(8:1)-C₁₇(8:1) ketone. The structure is confirmed by NMR.

### Synthesis of 3-(S)-2-(8Z)-pentadec-8-en-1-yl-2′-(8Z)-heptadec-8-en-1-yl-1,3-dioxolan-4-yl)-N,N-dimethylpropan-1-amine

The procedure for the synthesis of 3-((S)-2,2-di((Z)-heptadec-8-en-1-yl)-1,3-dioxolan-4-yl)-N,N-dimethylpropan-1-amine, AKG-KC3-C17(C8:1)), described above, is essentially followed using (7Z, 24Z)-tritriaconta-7,24-dien-16-one as a starting material. A cationic lipid having general structure I-A with one C₁₇ monounsaturated and one Cis monounsaturated R₁ hydrocarbon chain is obtained.

### Synthesis of (9Z)-pentatriacont-9-en-18-one (asymmetric C₁₇(8:1)-C₁₇ ketone)

An equimolar mixture of oleoyl chloride and stearoyl chloride is processed essentially as described above for the synthesis of (9Z,26Z)-pentatriaconta-9,26-dien-18-one (2). The products (C₁₇(8:1)-C₁₇(8:1), C₁₇(8:1)-C₁₇, and C₁₇-C₁₇ketones) are separated using column chromatography to isolate the asymmetric C₁₇(8:1)-C₁₇ ketone. The structure is confirmed by NMR.

### Synthesis of 3-(S)-2-(8Z)-heptadec-8-en-1-yl-2′-heptadec-1-yl-1,3-dioxolan-4-yl)-N,N-dimethylpropan-1-amine

The procedure for the synthesis of 3-((S)-2,2-di((Z)-heptadec-8-en-1-yl)-1,3-dioxolan-4-yl)-N,N-dimethylpropan-1-amine, AKG-KC3-C17(C8:1)), described above, is essentially followed using (9Z)-pentatriacont-9-en-18-one as a starting material. A cationic lipid having general structure I-A with one C₁₇ monounsaturated and one C₁₇ saturated (alkyl) R₁ hydrocarbon chain is obtained.

### Synthesis of (24Z)-tritriacont-24-en-16-one (asymmetric C₁₅-C₁₇(8:1) ketone)

An equimolar mixture of oleoyl chloride and palmitoyl chloride is processed essentially as described above for the synthesis of (9Z,26Z)-pentatriaconta-9,26-dien-18-one (2). The products (C₁₇(8:1)-C₁₇(8:1), C₁₅-C₁₅, and C₁₅-C₁₇(8:1) ketones) are separated using column chromatography to isolate the asymmetric C₁₅-C₁₇(8:1) ketone. The structure is confirmed by NMR.

### Synthesis of 3-(S)-2-(8Z)-pentadec-8-en-1-yl-2′-heptadec-1-yl-1,3-dioxolan-4-yl)-N,N-dimethylpropan-1-amine

The procedure for the synthesis of 3-((S)-2,2-di((Z)-heptadec-8-en-1-yl)-1,3-dioxolan-4-yl)-N,N-dimethylpropan-1-amine, AKG-KC3-C17(C8:1)), described above, is essentially followed using (24Z)-tritriacont-24-en-16-one as a starting material. A cationic lipid having general structure I-A with one C₁₇ monounsaturated and one C₁₅ saturated (alkyl) R₁ hydrocarbon chain is obtained.

### Example 2A. Preparation of Lipidic Nanoparticles (LNPs)

mRNA modified with 5-methoxyuridine (5moU) and coding for mCherry (Cat #L-7203) was obtained from Trilink Biotechnologies (San Diego, CA). All uridine nucleosides were substituted with N1-methyl-pseudouridine. To produce the mRNA, a synthetic gene encoding the mRNA sequence was cloned into a DNA plasmid. The synthetic gene was comprised of an RNA promoter, a 5′ untranslated region, mCherry protein coding sequence, a 3′ untranslated region, and a poly(A) tail region of approximately 120 As. The open reading frame sequence for the mCherry mRNA from TriLink (Cat #L-7203) corresponds to SEQ ID NO: 168:

AUGGUGAGCAAGGGCGAGGAGGACAACAUGGCCAUCAUCAAGGAG
UUCAUGCGGUUCAAGGUGCACAUGGAGGGCAGCGUGAACGGCCAC
GAGUUCGAGAUCGAGGGCGAGGGCGAGGGCCGGCCCUACGAGGGC
ACCCAGACCGCCAAGCUGAAGGUGACCAAGGGCGGCCCCCUGCCC
UUCGCCUGGGACAUCCUGAGCCCCCAGUUCAUGUACGGCAGCAAG
GCCUACGUGAAGCACCCCGCCGACAUCCCCGACUACCUGAAGCUG
AGCUUCCCCGAGGGCUUCAAGUGGGAGCGGGUGAUGAACUUCGAG
GACGGCGGCGUGGUGACCGUGACCCAGGACAGCAGCCUGCAGGAC
GGCGAGUUCAUCUACAAGGUGAAGCUGCGGGGCACCAACUUCCCC
AGCGACGGCCCCGUGAUGCAGAAGAAGACCAUGGGCUGGGAGGCC
AGCAGCGAGCGGAUGUACCCCGAGGACGGCGCCCUGAAGGGCGAG
AUCAAGCAGCGGCUGAAGCUGAAGGACGGCGGCCACUACGACGCC
GAGGUGAAGACCACCUACAAGGCCAAGAAGCCCGUGCAGCUGCCC
GGCGCCUACAACGUGAACAUCAAGCUGGACAUCACCAGCCACAAC
GAGGACUACACCAUCGUGGAGCAGUACGAGCGGGCCGAGGGCCGG
CACAGCACCGGCGGCAUGGACGAGCUGUACAAGAGCGGCAACUGA

Stock solutions of each lipid were prepared. Ionizable lipids were weighed out in 4 mL glass vials (Thermo B7999-2) and dissolved in ethanol (Sigma-Aldrich 200 proof, RNase free) to a final concentration of 10 mM. Other lipids such as DSPC, DPPC-NH₄, Cholesterol and PEG-DMG were weighed out and dissolved in ethanol to a concentration of 1 mM. DSPS-Na was dissolved in methanol (Sulpelco, Omnisolve) at a concentration of 1 mM and briefly heated to 70° C. to complete its dissolution.

Lipid mixtures for each individual LNP were prepared by adding the desired volume of each lipid stock solution to a new vial, adding ethanol if needed to achieve a final volume of 1.2 mL. For example, an LNP formulation of AKG-UO-1/DSPC/DSPS/Chol/PEG-DMG (50/2.5/7.5/38.5/1.5 mol %), with an N/P of 5 contained 1500 nmol AKG-UO-1, 75 nmol DSPC, 225 nmol DSPS, 1155 nmol Chol and 45 nmol PEG-DMG for every 100 g of mRNA used.

mRNA solutions were prepared by thawing frozen mRNA (mCherry mRNA, Trilink) vials and diluting mRNA in 6.25 mM sodium acetate (pH 5.0) to a final concentration of 0.033 mg/mL. To prepare LNPs, a NanoAssemblr Benchtop microfluidic device (from Precision Nanosystems) was used. If LNPs contained the sodium or ammonium salts of DSPS, or sodium salt of DPPS the heating block accessory set to 70° C. was used, otherwise LNPs were mixed at room temperature. 3 mL of mRNA solution was loaded into a 3 mL disposable syringe (BD 309656) and 1 ml of lipid mixture in a 1 ml syringe (BD309659) and placed in the NanoAssemblr heating block for 4 min prior to mixing. LNP formation was achieved by pumping the liquid streams through a disposable microfluidics cassette at 3:1 aqueous: alcohol volume ratio at 6 mL/min mixing speed. After mixing, 3.6 mL of LNP mixture was collected, while the initial mixed volume of 0.35 mL and last 0.05 mL of mix was discarded. Ethanol was removed by buffer exchange using SpectraPor dialysis tubing (12-14k MWCO) in PBS (Cytivia, SH30256.01) or by sequential concentration and dilution using Amicon Ultra-4 centrifugal concentrators (10k MWCO, at 500 g).

LNPs were typically exchanged into PBS, pH 7.4 and then 15 mM Tris, pH 7.4, 20% sucrose, concentrated to 20-50 ug/mL mRNA using an Amicon-Ultra 4 (100,000 MWCO) spin column, sterile filtered (Thermo Nalgene 0.2 um #720-1320) prior to freezing by immersion in liquid nitrogen for 5 min and long-term storage at −80° C.

### Example 2B: Generation of Lipid Nanoparticles (LNP) Containing mRNA

Stock solutions of each lipid were prepared. Ionizable lipids were weighed out in 4 mL glass vials (Thermo B7999-2) and dissolved in ethanol (Sigma-Aldrich 200 proof, RNase free) to a final concentration of 10 mM. Other lipids such as DSPC (Avanti Polar Lipids), Cholesterol (Dishman) and PEG-DMG (NOF) were weighed out and dissolved in ethanol to a concentration of 1 mM. DSPS-Na (NOF) was dissolved in methanol (Sulpelco, Omnisolve) at a concentration of 1 mM and briefly heated to 70° C. to complete its dissolution.

Lipid mixtures for each individual LNP were prepared by adding the desired volume of each lipid stock solution to a new vial, adding ethanol if needed to achieve a final volume of 1.2 mL. For example, a LNP formulation of AKG-UO-1/DSPC/DSPS/Chol/PEG-DMG (50/2.5/7.5/38.5/1.5 mol %), with an N/P of 5 contained 1500 nmol AKG-UO-1, 75 nmol DSPC, 225 nmol DSPS, 1155 nmol Chol and 45 nmol PEG-DMG for every 100 g of mRNA used. mRNA solutions were prepared by thawing frozen mRNA (SARS-CoV-2 spike mRNA, Vernal) vials and diluting mRNA in 6.25 mM sodium acetate (pH 5.0) to a final concentration of 0.033 mg/mL, where the concentration is confirmed by absorbance on a Nanodrop.

To prepare LNPs, a NanoAssemblr Benchtop microfluidic device (from Precision Nanosystems) was used. If LNPs contained DSPS, the heating block accessory set to 70° C. was used, otherwise LNPs were mixed at room temperature. 3 mL of mRNA solution was loaded into a 3 mL disposable syringe (BD 309656) and 1 ml of lipid mixture in a 1 ml syringe (BD309659) and placed in the NanoAssemblr heating block for 4 min prior to mixing. LNP formation was achieved by pumping the liquid streams through a disposable microfluidics cassette at 3:1 aqueous: alcohol volume ratio at 6 mL/min mixing speed. After mixing, 3.6 mL of LNP mixture was collected, while the initial mixed volume of 0.35 mL and last 0.05 mL of mix was discarded. Ethanol was removed by buffer exchange using SpectraPor dialysis tubing (12-14k MWCO) in PBS (Cytivia, SH30256.01). LNPs were typically exchanged into PBS, pH 7.4 and then 15 mM Tris, pH 7.4, 20% sucrose, concentrated to 20-50 ug/mL mRNA, sterile filtered (Thermo Nalgene 0.2 um #720-1320) prior to freezing by immersion in liquid nitrogen for 5 min and long-term storage at −20° C. For this study, samples were concentrated to >40 μg/mL mRNA, and diluted with varying volumes of 15 mM Tris, 20% Sucrose, pH 7.4 to a target concentration of 40 μg mRNA and then frozen on LN2. Characterization of LNPs was undertaken after an aliquot of the LNPs were thawed and diluted 1:1 (vol:vol) with 15 mM Tris, pH 7.4 such that the final concentration was 20 μg/mL mRNA in 15 mM Tris, 10% sucrose, pH 7.4. This simulated the conditions of sample preparation that were performed prior to dosing the animals with an injection of 1 μg mRNA in 50 μL volume via IM injection into a hind limb.

LNP Characterization. mRNA encapsulation and mRNA concentration within the LNPs was measured using a Ribogreen assay. Nanoparticle size and zeta potential were measured by a zetasizer (Malvern).

### Example 3. LNP Characterization

A. mRNA Concentration and Relative Encapsulation Efficiency Determination by Fluorescent Binding Dye

Materials: Ribogreen reagent (Thermo #11491), 3×96-well plates with lids, PBS, dissociation buffer (PBS with 10% DMSO and 1% (wt/wt) Zwittergent 3-14 (Sigma-Aldrich #693017), mRNA, general pipette tips & repeater pipette tips.
1. 5 mL of 2 g/mL mRNA stock were prepared in DPBS or PBS
2. Diluted standards were prepared as follows in single wells in a 96-well plate (Plate A);

| Final [mRNA] ng/mL | Vol. stock 2 μg/mL (μL) | Vol. PBS (μL) |
|---|---|---|
| 2000 | 400 | 0 |
| 1500 | 300 | 100 |
| 1000 | 200 | 200 |
| 500 | 100 | 300 |
| 0 | 0 | 400 |

3. Using different wells in Plate A, sample mRNA concentration was estimated and were diluted to be within the standard curve. For example, if the approximate mRNA concentration should be ˜30 μg/mL in the sample, a 20× dilution was performed (Dilution Factor). (20 uL sample added to 380 μL PBS in a well). No lid was used on plate A. Samples were mixed by gentle pipetting up & down.

### Example of Plate A

| | | | | | | | | | |
|---|---|---|---|---|---|---|---|---|---|
| A | 0 | 500 | 1000 | 1500 | 2000 | | | | |
| B | | | | | | | | | |
| C | S 1 | S 2 | S 3 | S 4 | S 5 | S 6 | S 7 | S 8 | Etc. |
| D | | | | | | | | | |
| E | | | | | | | | | |
| F | | | | | | | | | |
| G | | | | | | | | | |
| H | | | | | | | | | |

4. Two more plates, plates B & C were used. Using a multichannel pipettor, 60 μL of each standard 2 were pipetted into wells each (duplicate), and sample into 3 wells each (triplicate) Example of Plate B and C

| | | | | | | | | | |
|---|---|---|---|---|---|---|---|---|---|
| A | 0 | 500 | 1000 | 1500 | 2000 | | | | |
| B | 0 | 500 | 1000 | 1500 | 2000 | | | | |
| C | S 1 | S 2 | S 3 | S 4 | S 5 | S 6 | S 7 | S 8 | Etc. |
| D | S 1 | S 2 | S 3 | S 4 | S 5 | S 6 | S 7 | S 8 | Etc. |
| E | S 1 | S 2 | S 3 | S 4 | S 5 | S 6 | S 7 | S 8 | Etc. |
| F | | | | | | | | | |
| G | | | | | | | | | |
| H | | | | | | | | | |

5. The number of wells used on each plate was counted and 4 was added to this number. For plate B, PBS was prepared with Ribogreen diluted 1:100. For example, for 40 wells, 44 was used as the number. 44×60 μL=2.64 mL Ribogreen solution needed, so that would be 2.61 mL PBS with 26.4 μL Ribogreen.
6. For plate C, 2.61 mL Dissociation buffer and 26.4 uL Ribogreen was pipetted.
7. Using a repeater pipette set for 60 μL, PBS+RiboGreen was added to each well on plate B and 60 μL Dissociation Buffer+Ribogreen to plate C. Both plates B and C were mixed on an orbital mixer (120 rpm) for 1 min. Plate B was placed in the dark for 15 min. Plate C was incubated at 37° C. in the dark for 10 min, followed by 5 min at RT.
8. Both plates were read one after the other, using Ex. 485, Em. 530 nm
9. Using the standard curve, the slope and intercept were calculated and by extrapolation the mRNA concentrations of the samples on plate B & C were calculated (average and std.dev)
10. Percent encapsulation efficiency (% EE) by [mRNA] plate B/[mRNA] plate C×100 was calculated
11. Total [mRNA] by taking [mRNA] plate C X dilution factor was calculated.

### B. LNP Particle Size

1. 30 μL of LNP was mixed with 1.5 mL PBS in a polystyrene cuvette (Sarstedt, #67.754) and analyzed for size using a ZetaSizer Pro (Malvern) using ZS Xplorer software, version number 1.4.0.105. The Z-average size and polydispersity index value were recorded. Typically, size measurements of LNPs were taken post LNP mixing, post buffer exchange and post sterile filtering.

### C. LNP Zeta Potential

1. 30 μL of LNP was mixed with 1.5 mL PBS and injected into a disposable folded capillary cell (Malvern Nanoseries DTS1070) and zeta potential measured on a ZetaSizer Pro at 25° C.

### Example 4. Determination of Transfection Efficiency in Murine Dendritic Cells of LNPs Using mCherry mRNA

### A. Cell Propagation, Transfection, Harvesting and Staining Protocol

1. MutuDC1940 cells (Applied Biological Materials, T0528) were grown according to supplier's instructions in T75 flasks. They were plated at 180,000 cells/well into 24-well plate one day prior to transfection.
2. LNPs were added in triplicate to each well at the desired mRNA concentration (e.g. 1 μg/mL) in 1 mL media and after 24 h the cells were washed once with DPBS (VWR 02-0119-1000).
3. 0.2 mL of DPBS (plus 5 mM EDTA, pH 7.4) was then added to facilitate detachment.
4. The cells were placed at 37° C. for 3 min, until detached.
5. 0.5 ml DPBS added to each well and the liquid transferred to a flow cytometry tube (Falcon 5 mL #352054).
6. The tube was centrifuged at 1100 rpm for 3-5 min and the liquid poured off.
7. 100 μL of Zombie Violet (Biolegend) (diluted 1:500 in PBS) was added to each tube.
8. The tubes were gently tapped to resuspend cells and placed in the dark for 15 min at RT.
9. To the cells 0.5 mL of (paraformaldehyde 4% in PBS:DPBS 1:1) was added and the cells flicked gently to resuspend and put on ice for 30 min. Another 2 ml PBS was added.
10. The cells were pelleted as above and resuspended in 0.5 mL DPBS with 5% BSA and placed in the fridge until needed.

### B. Cell Analysis

Cells suspensions were analyzed by an Attune NxT flow cytometer using the VL1 and YL2 for live/dead and mCherry fluorescence signals respectively. Gating analysis was performed on FloJo software.

### Example 5. Additional Exemplary Phosphatidyl-L-Serine Targeted LNP Formulations

Additional formulations shown below could also be prepared using the methods described in the Examples above. All lipid concentrations are shown as mol % as a percentage of the total lipid in the LNP. The below compositions vary in conjugated lipid content from 0.5 to 2.5 mol %, in sterol content from 25-45 mol %, in ICL content from 40-65 mol %, in saturated phosphatidyl-L-serine content from 2-10 mol %, and in total noncationic phospholipid content from 5-20 mol %. Most compositions would contain two phospholipids, typically phosphatidyl-L-serine (DSPS or DPPS being preferred) and phosphatidylcholine, although some exemplary formulations may contain more than two phospholipids, including phosphatidylethanolamines, like dioleoylphosphatidylethanolamine (DOPE).

TABLE 15
Exemplary phosphatidyl-L-serine containing LNP formulations

| | Ionizable | | Additional | | Conjugated |
|---|---|---|---|---|---|
| Formulation | Cationic Lipid | PS | Phospholipid(s) | Sterol | lipid |
| (#) | (mol %) | (mol %) | (mol %) | (mol %) | (mol %) |
| 1 | KC3-0A (47.5) | DSPS (7.5) | DSPC (3.5) | Chol (40) | PEG-DMG (1.5) |
| 2 | KC3-0A (45) | DSPS (8) | DSPC (4) | Chol (42) | PEG-DMG (1) |
| 3 | AKG-KC2-01 (40) | DSPS (5) | DSPC (5) | Chol (44) | PEG-DMG (1) |
| | | | DOPE (5) | | |
| 4 | KC3-PA (42.5) | DSPS (7.5) | DSPC (6.5) | Chol (42) | PEG-DMG (1.5) |
| 5 | AKG-KC3-01 (65) | DSPS (7.5) | DSPC (2.5) | Chol (24.5) | PEG-DMG (0.5) |
| 6 | AKG-KC32-01 (60) | DSPS (6) | DSPC (4) | Chol (29) | PEG-DMG (1) |
| 7 | KC3-PA (55) | DSPS (7) | DSPC (3) | Chol (34) | PEG-DMG (1) |
| 8 | AKG-KC2-01 (57) | DSPS (6.5) | DSPC (3.5) | Chol (28) | PEG-DMG (1) |
| 9 | AKG-KC2-01 (60) | DSPS (7.5) | DSPC (2.5) | Chol (28.5) | PEG-DMG (1.5) |
| | | | DOPE (4) | | |
| 10 | AKG-KC2-01 (48) | DSPS (6) | DSPC (4) | Chol (41.5) | PEG-DMG (0.5) |
| 11 | KC3-PA (48) | DPPS (6) | DSPC (4) | Chol (41.5) | PEG-DMG (0.5) |
| 12 | AKG-KC2-01 (48) | DSPS (6) | DPPC (4) | Chol (41.5) | PEG-DMG (0.5) |
| 13 | AKG-KC2-PA (47.5) | DSPS (6) | DSPC (4) | Chol (41.5) | PEG-DMG (1) |
| 14 | KC3-PA (47.5) | DPPS (6) | DSPC (4) | Chol (41.5) | PEG-DMG (1) |
| 15 | AKG-KC2-PA(47.5) | DSPS (6) | DPPC (4) | Chol (41.5) | PEG-DMG (1) |
| 16 | KC3-0A (48) | DSPS (7.5) | DSPC (2.5) | Chol (41.5) | PEG-DMG (0.5) |
| 17 | KC3-C17(8:1) | DPPS (7.5) | DSPC (2.5) | Chol (41.5) | PEG-DMG (0.5) |
| | (48) | | | | |
| 18 | KC3-C17(8:1) | DSPS (7.5) | DPPC (2.5) | Chol (41.5) | PEG-DMG (0.5) |
| | (48) | | | | |
| 19 | KC3-0A (47.5) | DSPS (7.5) | DSPC (4.5) | Chol (39) | PEG-DMG (1.5) |
| 20 | KC3-0A (47.5) | DPPS (7.5) | DSPC (4.5) | Chol (39) | PEG-DMG (1.5) |
| 21 | KC3-0A (47.5) | DSPS (7.5) | DSPC (2.5) | β-sitosterol (42) | PEG-DMG (0.5) |
| 22 | KC3-0A (50) | DSPS (7.5) | DSPC (2.5) | β-sitosterol (39) | PEG-DMG (1) |
| 23 | KC3-0A (52.5) | DSPS (7.5) | DSPC (2.5) | β-sitosterol (39) | PEG-DMG (1) |
| 24 | KC3-0A (55) | DSPS (7.5) | DSPC (2.5) | β-sitosterol (39) | PEG-DMG (1) |
| 25 | KC3-0A (55) | DSPS (7.5) | DSPC (2.5) | Chol (34) | PEG-DMG (1) |
| 26 | KC3-0A (57) | DSPS (6.5) | DSPC (3.5) | Chol (28) | PEG-DMG (1) |
| | | | DOPE (4) | | |
| 27 | KC3-0A (52.5) | DSPS (7.5) | DSPC (6.5) | Chol (28) | PEG-DMG (1.5) |
| | | | DOPE (4) | | |
| 28 | KC3-0A (48) | DSPS (8) | DSPC (2) | Chol (41.5) | PEG-DMG (0.5) |
| 29 | KC3-PA (48) | DSPS (7) | DSPC (3) | Chol (41) | PEG-DMG (1) |
| 30 | AKG-KC3-01 (45) | DSPS (6) | DSPC (4) | Chol (44) | PEG-DMG (1) |
| 31 | KC3-PA (42.5) | DSPS (7.5) | DSPC (6.5) | Chol (42) | PEG-DMG (1.5) |
| 32 | KC3-0A (48) | DPPS (8) | DSPC (2) | Chol (41.5) | PEG-DMG (0.5) |
| 33 | KC3-C17(8:1) | DSPS (5) | DSPC (5) | Chol (41.5) | PEG-DMG (0.5) |
| | (48) | | | | |
| 34 | KC3-C17(8:1) | DSPS (7.5) | DSPC (2.5) | Chol (41) | PEG-DMG (1) |
| | (48) | | | | |

TABLE 15
Exemplary phosphatidyl-L-serine containing LNP formulations (continued)

| | Ionizable | | Additional | | Conjugated |
|---|---|---|---|---|---|
| Formulation | Cationic Lipid | PS | Phospholipid(s) | Sterol | lipid |
| (#) | (mol %) | (mol %) | (mol %) | (mol %) | (mol %) |
| 35 | KC3-C17(8:1) | DSPS (5) | DSPC (5) | Chol (39) | PEG-DMG (1) |
| | (50) | | | | |
| 36 | KC3-C17(8:1) | DSPS (7.5) | DSPC (2.5) | Chol (39) | PEG-DMG (1) |
| | (50) | | | | |
| 37 | KC3-OA (48) | DSPS (5) | DSPC (5) | Chol (41.5) | PEG-DMG (0.5) |
| 38 | ALC-0315 (46) | DSPS (5) | DSPC (5) | Chol (43) | PEG-DMG (1) |
| 39 | ALC-0315 (48) | DSPS (5) | DSPC (5) | Chol (41) | PEG-DMG (1) |
| 40 | SM-102 (50) | DSPS (5) | DSPC (5) | Chol (39.5) | PEG-DMG (0.5) |
| 41 | SM-102 (50) | DSPS (7.5) | DSPC (2.5) | Chol (39) | PEG-DMG (1) |
| 42 | SM-102 (48) | DSPS (5) | DSPC (5) | Chol (41) | PEG-DMG (1) |
| 43 | KC3-C17(8:1) | DSPS (7.5) | — | Chol (43.5) | PEG-DMG (1) |
| | (48) | | | | |
| 44 | KC3-PA (48) | DSPS (7.5) | — | Chol (43.5) | PEG-DMG (1) |
| 45 | AKG-KC3-01 (48) | DSPS (5) | DSPC (5) | Chol (26) | PEG-DMG (1) |
| | | | DOPE (15) | | |
| 46 | AKG-KC3-01 (48) | DSPS (5) | DSPC (5) | Chol (31) | PEG-DMG (1) |
| | | | DOPE (10) | | |
| 47 | AKG-KC3-01 (48) | DSPS (6) | DSPC (4) | Chol (40) | PEG-DMG (2) |
| 48 | AKG-KC3-01 (48) | DSPS (6) | DSPC (4) | Chol (39.5) | PEG-DMG (2.5) |
| 49 | KC3-PA (46) | DSPS (7) | DSPE (5) | Chol (41.5) | PEG-DMG (0.5) |
| 50 | KC3-PA (48) | DSPS (7.5) | eggSM (2.5) | Chol (41.5) | PEG-DMG (0.5) |
| 51 | AKG-KC3-01 (48) | DSPS (5) | HSPC (5) | Chol (41.5) | PEG-DMG (0.5) |
| 52 | KC3-PA (47.5) | DSPS (7.5) | DSPC (2.5) | Chol (42) | PEG-DPG (0.5) |
| 53 | KC3-PA (47) | DSPS (7.5) | DSPC (2.5) | Chol (42) | PEG-DPG (1) |

### Example 6. Determination of Transfection Efficiency in Human Dendritic Cells of LNPs Using mCherry mRNA

Four days prior to transfection, monocytes were isolated using CD14 isolation kit (StemCell) from PBMCs of healthy donors. Purity CD14=90.3%, viability=93.2%. The monocytes were cultured with IL-4 (R&D 1000 IU/mL) and GM-CSF (R&D, 800 IU/mL) in a 6 well dish at 1×106 cells/mL at 37° C., 5% CO2. After 4 days the immature DCs were harvested and seeded in a 96 well round bottom plate at 50,000 cells/well. LNPs were thawed by placing the vials in a 37° C. water bath for 30 seconds, or until the sample had almost fully melted. The vials were immediately placed on ice until use. The LNPs were added to final concentrations of 1 μg/ml and 0.1 μg/mL mRNA. For 1 μg/mL treatment, LNPs were added directly to each well then wells were mixed by pipetting. For 0.1 μg/mL treatment, LNPs were diluted 1:10 in complete media then added to each well and mixed by pipetting. After 4 h a maturation cytokine cocktail was added directly to each well consisting of TNF-α (R&D, 10 ng/mL), IL-1b (R&D, 2 ng/mL), IL-6 (R&D, 1000 IU/mL), and PGE1 (R&D, 1 μg/mL). After 24 h, the cells were centrifuged, washed in PBS and analyzed by flow cytometry for mCherry fluorescence. Gating analysis was performed using CytExpert software.

### Example 7. Stability of Ionizable Cationic Lipids Under Accelerated Oxidation Conditions

The aim of this studies was to compare stability of ionizable cationic lipids under accelerated oxidation.

Ethanol (Sigma-Aldrich, cat #459836) stocks of 1 mM cationic lipids (KC3, KC3-OA, KC3-PA, KC3-C17 and KC3-C17(C8:1)) were prepared and stored at −20° C. Prior to the experiment, 75 μl of a lipid stock was mixed with 75 μl of ultra-pure water (Rx Biosciences, cat #P01-UPW02-1000) to make 0.5 mM of cationic lipids and 5 mM of linoleic acid respectively. A combined stock in water of 10% H₂O₂ (Sigma-Aldrich, cat #H1009) and 1 mM of Fe(III)Cl (Sigma-Aldrich, cat #372870) was freshly by prepared prior to the experiment. To make 1% final concentration of H₂O₂ and 100 μM Fe(III)Cl, 15 μl of 10% H₂O₂/1 mM Fe(III)C₁ stock was added to 135 μl of individual lipids The liposomes and individual lipids were incubated with H₂O₂/Fe(III)C₁ at 37° C. and then 5 μl from each sample was taken at different time points (0, 24, 48, 72 and 96 hours) and dissolved in 90 μl of MeOH for HPLC analysis. Degradation of the main lipid peak was analyzed using Thermo Scientific Vanquish Flex UHPLC occupied with Charged Aerosol Detector (CAD) and Phenomenex™ Kinetex C8 column (L=150 mm, D=2.1 mm, Particle Size=1.7 μm, 100 A). The UHPLC operating conditions are listed in Table 24.

TABLE 16
Chromatographic Conditions

| | |
|---|---|
| HPLC Instrument | Thermo Scientific Vanquish Flex UHPLC |
| HPLC Column | PhenomenexTM Kinetex C8 column (L = 150 mm, |
| | D = 2.1 mm, Particle Size = 1.7 μm, 100 A) |
| Column | 50° C. |
| Temperature | |
| Flow Rate | 0.5 mL/min |
| Injection Volume | 5 μL |
| CAD | 10 Hz |
| Run Time | 11 min |
| Sample | 21° C. |
| Temperature | |
| Sample Solvent | MeOH |
| Mobile Phase | Mobile Phase A: 100 mM ammonium acetate |
| | in water (pH 4) |
| | Mobile Phase B: Methanol |

Mobile Phase Program:

| Time, | Mobile | Mobile |
|---|---|---|
| min | Phase A,% | Phase B,% |
| −0.5 | 18 | 82 |
| 0 | 18 | 82 |
| 1 | 16 | 84 |
| 4 | 10 | 90 |
| 6 | 0 | 100 |
| 9 | 0 | 100 |
| 10 | 18 | 82 |
| 11 | 18 | 82 |

The data are presented as a percentage of the main lipid peak measured at different time points relative to the lipid peak measured at time zero.

As shown in FIG. 1 and Table 17, KC3 demonstrated the fastest degradation relative to other cationic lipids under the forced oxidation with H₂O₂.

TABLE 17
Effect of hydrogen peroxide on the stability
of individual ionizable cationic lipids

% of main lipid peak to Tₒ

| Lipid | 24 h. | 48 h. | 72 h. |
|---|---|---|---|
| KC3 | 11.2 ± 1.0 | 10.5 ± 0.5 | 0 ± 0 |
| KC3-OA | 88.9 ± 0.5 | 84.6 ± 0.9 | 79.0 ± 0.1 |
| KC3-PA | 62.7 ± 0.1 | 59.2 ± 0.8 | 60.2 ± 0.6 |
| KC3-17 | 92.9 ± 0.3 | 91.8 ± 1.2 | 90.2 ± 1.2 |
| KC3-17 (C8:1) | 66.1 ± 0.6 | 58.4 ± 1.5 | 45.9 ± 4.7 |

### Example 8. Comparison of KC2 and KC3-Family Lipids in PS-Targeted LNP Expression Murine Dendritic Cells

The aim of this study was to explore the effect of different KC2 and KC3 ICLs on transfection efficiency in murine dendritic cells. The KC2 (DLin-KC2-DMA) and KC3 (DLin-KC3-DMA) lipids both have linoleic chains with two olefins each but differ in the ionizable amine group coming off the dioxolane ring, with KC2 having a dimethylaminoethyl group at this position and KC3 has a dimethylaminopropyl group at this position. Two other variants evaluated have monounsaturated alkyl chains with KC3-OA being 18 carbons in length and KC3C17(8:1) having 17 carbons. LNPs were prepared as described in Example 2A, characterized for particle size and zeta potential as described in Example 3, and evaluated for transfection efficiency in murine dendritic cells as described in Example 4. The LNPs used the ionizable lipids in Tables 29-30 in the ICL with a constant N/P ratio of 5.25, the DPPS (NH4+ salt) lipid was either 0 or 5 mol % and the DSPC phospholipid constant at 10 or 5 mol % (total of DPPS and DSPC was 10 mol %), and the cholesterol constant at 38.5 mol %.

TABLE 18
Physicochemical properties of LNPs used in Example 8.

| | | Particle | |
|---|---|---|---|
| | | Size (nm) | |
| | Particle | Post- | Encapsulation |
| LNP formulation | Size (nm) | Freeze/Thaw | Efficiency (%) |
| KC2/DSPC/DSPS | 87.8 | | 85.6 ± 4.6 |
| KC3/DSPC | 91.2 | 90.1 | 95.1 ± 5.6 |
| KC3/DSPC/DPPS | 90.5 | 90.2 | 95.0 ± 9.8 |
| KC3-OA/DSPC | 88.4 | 84.7 | 96.0 ± 13.1 |
| KC3-OA/DSPC/DPPS | 97.7 | 97.7 | 96.5 ± 7.3 |
| KC3C17(8:1)/DSPC | 85.3 | 83.0 | 94.4 ± 6.2 |
| KC3C17(8:1)/DSPC/DPPS | 100.2 | 96.8 | 94.7 ± 8.4 |
| KC3C17/DSPC | 73.9 | 77.5 | 95.6 ± 5.5 |
| KC3C17/DSPC/DPPS | 85.2 | 81.4 | 97.0 ± 8.4 |

TABLE 19
mCherry expression in murine dendritic cells

| | [mRNA] | | | vs KC3 |
|---|---|---|---|---|
| LNP formulation | (μg/mL) | MFI | T/NT | LNP (%) |
| Untreated (UT) | | 234 ± 6 | | |
| KC2/DSPC/DSPS | 1 | 13,570 ± 2,324 | | 17.3% |
| KC3/DSPC | 1 | 14,962 ± 711 | | |
| | 0.3 | 6,656 ± 125 | | |
| KC3/DSPC/DPPS | 1 | 78,539 ± 15,239 | 5.32 | |
| | 0.3 | 30,525 ± 2,946 | 4.72 | |
| KC3-OA/DSPC | 1 | 27,513 ± 10,579 | | 185% |
| | 0.3 | 12,719 ± 1,316 | | 194% |
| KC3-OA/DSPC/DPPS | 1 | 163,844 ± 24,614 | 6.00 | 209% |
| | 0.3 | 85,584 ± 10,863 | 6.84 | 282% |
| KC3C17(8:1)/DSPC | 1 | 16,075 ± 1,956 | | 108% |
| | 0.3 | 6,701 ± 440 | | 101% |
| KC3C17(8:1)/DSPC/DPPS | 1 | 145,159 ± 35,387 | 9.15 | 185% |
| | 0.3 | 61,788 ± 7,404 | 9.52 | 203% |
| KC3C17DSPC | 1 | 344 ± 15 | | 0.8% |
| | 0.3 | 270 ± 8 | | 0.6% |
| KC3C17/DSPC/DPPS | 1 | 428 ± 30 | 1.77 | 0.3% |
| | 0.3 | 330 ± 5 | 2.64 | 0.3% |

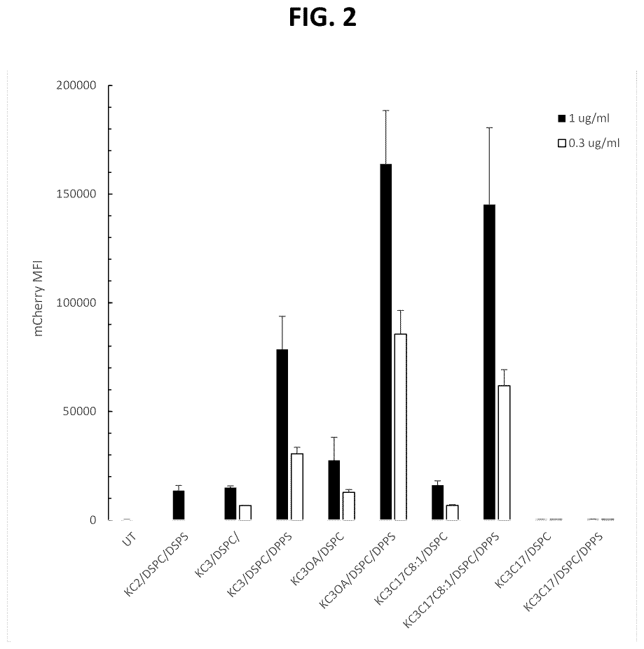

The effect of targeting and ICL choice on transfection activity was evaluated using both nontargeted and 5 mol % DSPS-targeted LNPs (FIG. 2). The five ICLs used in this study had either a diacyl structure that varied in the specific ionizable amine used, and thus it's apparent pKa. This data shows that the two KC3 lipids with a single olefin, KC3OA and KC3C17(8:1) showed the highest activity when incorporated into LNPs. These two lipids when incorporated into 5 mol % DPPS-targeted LNPs showed between 185-282% of the activity of the KC3 lipid containing LNP formulation and 1,087-1,227% of the transfection activity of LNPs containing the KC2 lipid. Previously, Semple and colleagues (Semple et al., (2010) Nature Biotech. 28 (2) 172-176) had shown that KC2 was 6-fold more active compared to KC3, and all the lipids were designed with the understanding that linoleyl lipids with two cis double bonds would be significantly less active based on prior work with DLinDMA (Heyes et al., (2005) J Control Release 107, 276-287), and thus monounsaturated lipids were never evaluated. Here, the data clearly show using two different monounsaturated ICLs, KC3-OA and KC3C17(8:1), that the monounsaturated lipids were clearly superior to lipids containing dilioleyl chains and compared to those containing fully saturated alkyl chains (KC3C17), which was completely inactive, even in the presence of DPPS targeting lipid. The data also show these new lipids were significantly more active than the lead, and most active lipid, KC2 (DLin-KC2-DMA) arising from this early work. Finally, LNPs comprised of both DPPS and one of these two monounsaturated ICLs, showed a 6-10 fold targeting effect compared to similar LNPs without DPPS.

### Example 9. Comparison of KC2 and KC3-Family Lipids in PS-Targeted LNP Expression Human Dendritic Cells

The aim of this study was to explore the effect of different KC2 and KC3 ICLs on transfection efficiency in human dendritic cells. The KC2 (DLin-KC2-DMA) and KC3 (DLin-KC3-DMA) lipids both have linoleic chains with two olefins each but differ in the ionizable amine group coming off the dioxolane ring, with KC2 having a dimethylaminoethyl group at this position and KC3 has a dimethylaminopropyl group at this position. Three other variants evaluated have monounsaturated alkyl chains with KC3-OA being 18 carbons in length, KC3-PA having 16 carbons, and KC3C₁₇(8:1) having 17 carbons. The aim of this study was also to evaluate the impact of the PC component on transfection by comparing the fully saturated C16 DPPC and the fully saturated DSPC using both KC3-OA and KC3C17(8:1) containing LNPS. LNPs were prepared as described in Example 2A, characterized for particle size and zeta potential as described in Example 3, and evaluated for transfection efficiency in human dendritic cells as described in Example 6. The LNPs used the ionizable lipids in Tables 31-33 with the ICL held constant at an N/P ratio of 5.25 and 48 mol 0, the DPPS (NH4+ salt) lipid was included at mol 0 and the DSPC phospholipid constant at 9 mol 0 (total of 0DPPS and DSPC/DPPC was 10 mol 3), and the cholesterol constant at 38.5 mol % The final ALC-0315/DSPC formulation was composed of 46.3 mol 0 ALC-0315, 9.4 mol 0 DSPC, 42.7 mol 0 cholesterol and 1.56 mol % PEG-DMG with a N/P of 6.2. The final SM-102/DSPC formulation was composed of 50 mol 9 SM-102, 10 mol % DSPC, 38.5 mol % cholesterol and 1.5 mol % PEG-DMG with a N/P of 5.

TABLE 19
Physicochemical properties of LNPs used in Example 9.

| | | Particle | |
|---|---|---|---|
| | | Size (nm) | |
| | Particle | Post- | Encapsulation |
| LNP formulation | Size (nm) | Freeze/Thaw | Efficiency (%) |
| KC2/DSPC/DSPS | 71.8 | 69.6 | 85.4 ± 1.5 |
| KC3-01/DSPC/DPPS | 67.9 | 71.7 | 84.3 ± 3.6 |
| KC3/DSPC/DPPS | 103.4 | 100.3 | 91.5 ± 6.2 |
| KC3-OA/DSPC/DPPS | 87.9 | 87.7 | 92.2 ± 8.6 |
| KC3-OA/DPPC/DPPS | 89.8 | 98.2 | 91.7 ± 6.3 |
| KC3-PA/DSPC/DPPS | 104.7 | 98.0 | 90.1 ± 10.3 |
| KC3C17(8:1)/DSPC/DPPS | 91.6 | 92.7 | 91.7 ± 5.3 |
| KC3C17(8:1)/DPPC/DPPS | 86.6 | 88.7 | 92.9 ± 2.6 |
| ALC-0315/DSPC | 69.8 | 71.5 | 88.0 ± 4.1 |
| SM-102/DSPC | 82.1 | 83.4 | 92.4 |

TABLE 20
mCherry expression in human dendritic
cells following incubation at 1 μg/ml

| | | vs KC3 | vs DPPC |
|---|---|---|---|
| | | LNP | LNP |
| LNP formulation | MFI | (fold) | (fold) |
| Untreated (UT) | 2,279 ± 129 | | |
| KC2/DSPC/DSPS | 11,150 ± 1,556 | 0.08 | |
| KC3-01/DSPC/DPPS | 1,149,875 ± 111,037 | 10.08 | 2.35 |
| KC3-OA/DSPC/DPPS | 963,929 ± 95,910 | 8.44 | |
| KC3-OA/DPPC/DPPS | 412,204 ± 37,299 | 3.60 | |
| KC3-PA/DSPC/DPPS | 757,849 ± 95,345 | 6.64 | |
| KC3C17(8:1)/DSPC/DPPS | 534,708 ± 80,921 | 4.68 | 3.60 |
| KC3C17(8:1)/DPPC/DPPS | 149,980 ± 34,195 | 1.30 | |
| KC3/DSPC/DPPS | 116,155 ± 30,093 | 1.00 | |
| ALC-0315/DSPC | 10,327 ± 1,916 | 0.07 | |
| SM-102/DSPC | 258,967 ± 16,828 | 2.25 | |

TABLE 21
mCherry expression in human dendritic
cells following incubation at 0.1 μg/ml

| | | vs KC3 | vs DPPC |
|---|---|---|---|
| | | LNP | LNP |
| LNP formulation | MFI | (fold) | (fold) |
| Untreated (UT) | 2,502 ± 293 | | |
| KC2/DSPC/DSPS | 3,048 ± 193 | 0.09 | |
| KC3-01/DSPC/DPPS | 46,489 ± 4,173 | 7.45 | |
| KC3-OA/DSPC/DPPS | 44,197 ± 4,407 | 7.06 | 2.61 |
| KC3-OA/DPPC/DPPS | 18,485 ± 1,488 | 2.71 | |
| KC3-PA/DSPC/DPPS | 35,177 ± 3,396 | 5.53 | |
| KC3C17(8:1)/DSPC/DPPS | 25,044 ± 2,144 | 3.82 | 2.82 |
| KC3C17(8:1)/DPPC/DPPS | 10,499 ± 979 | 1.35 | |
| KC3/DSPC/DPPS | 8,409 ± 660 | 1.00 | |
| ALC-0315/DSPC | 3,783 ± 1,090 | 0.22 | |
| SM-102/DSPC | 9,177 ± 1,494 | 1.13 | |

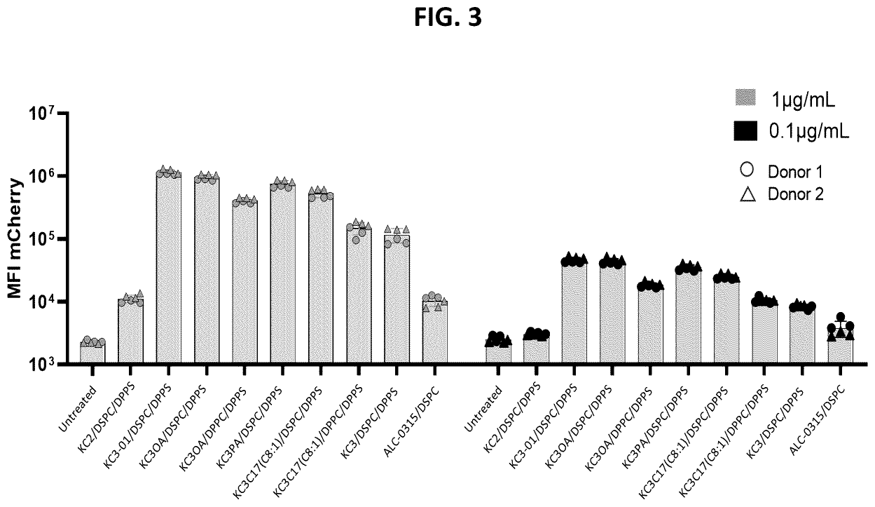

The effect of targeting and ICL choice on transfection activity was evaluated using both nontargeted and 5 mol % DPPS-targeted LNPs (FIG. 3). The five ICLs used in this study had either a diacyl structure that varied in the specific ionizable amine used. This data shows that the three KC3 lipids with a single olefin, KC3OA, KC3-PA and KC3C₁₇(8:1), or the polyunsaturated KC3-01 lipid with four methylenes between the two olefins showed the highest activity when incorporated into LNPs (Tables 20 and 21). The four lipids showed a 4.7-10.8 fold increase in transfection activity compared to KC3-containing LNPs in human dendritic cells at 1 μg/ml and 3.8-7.5 fold increase at 0.1 μg/mL. The increase was a further 10-12 fold higher when compared to targeted LNPs containing KC2. Both KC2 and KC3 containing linoleoyl alkyl chains (two olefins separated by one methylene). Previously, Semple (Semple et al., (2010) Nature Biotech. 28 (2) 172-176) had shown that KC2 was 6-fold more active compared to KC3, and all the lipids were designed with the understanding that linoleyl lipids with two cis double bonds would be significantly less active based on prior work with DLinDMA (Heyes et al., (2005) J Control Release 107, 276-287), and thus monounsaturated lipids were never evaluated. Here, the data clearly show using three different monounsaturated ICLs, KC3-OA, KC3-PA, and KC3C17(8:1), that the monounsaturated lipids were clearly superior to lipids containing dilioleyl chains. However, the largest improvement at both concentrations came when using the KC3-01 ICL with four methylenes between two olefins. The data also show these new lipids were significantly more active than the lead, and most active lipid, KC2 (DLin-KC2-DMA) arising from this early work and compared to LNPs containing either ALC-0315 or SM-102. Finally, two of the DPPS-targeted LNPs comprised of both DPPS and either KC3-OA or KC3-C17(8:1) ICLs, were evaluated with either the C16 DPPC or the C18 DSPC as the phosphatidylcholine component. For both sets of formulations, the DSPC-containing formulation showed higher transfection activity in human dendritic cells compared to the DPPC formulation, 2.61-2.82-fold higher 0.1 μg/mL and 2.35-3.60-fold higher at 1 μg/mL. This suggests that the DSPC containing LNPs provide for greater transfection activity when compared to lower phase transition lipids like DPPC.

### Example 10. Impact of ICL Concentration in PS-Targeted LNPs on Expression in Human Dendritic Cells

The aim of this study was to explore the effect of KC3-OA concentration in DSPS-targeted LNPs on transfection efficiency in human dendritic cells. The concentrations of KC3-OA were varied from 46-54 mol %, while keeping the DSPS and DSPC concentrations constant at 5 mol % each and PEG-DMG at 1.5 mol %. The increase in % KC3OA was commensurate with a proportional reduction in the % of cholesterol. ALC-0315 and SM-102 comparator formulations were also evaluated. The final ALC-0315/DSPC formulation was composed of 546.3 mol 0 ALC-0315, 9.4 mol 0 DSPC, 42.7 mol 0 cholesterol and 1.56 mol % PEG-DMG with a N/P of 6.2. The final SM-102/DSPC formulation was composed of 50 mol SM-102, 10 mol v DSPC, 38.5 mol % cholesterol and 1.5 mol % PEG-DMG with a(N/P of 5.

LNPs were prepared as described in Example 2A, characterized for particle size and zeta potential as described in Example 3, and evaluated for transfection efficiency in human dendritic cells as described in Example 6.

TABLE 22
Physicochemical properties and characterization of DSPS-targeted
KC3-OA LNPs containing various concentrations of KC3-OA

| | | Particle | |
|---|---|---|---|
| | | Size (nm) | |
| | Particle | Post- | Encapsulation |
| LNP formulation | size (nm) | Freeze/Thaw | Efficiency (%) |
| SM-102/DSPC | 71.0 | 74.1 | 84.1 ± 10.7 |
| ALC-0315/DSPC | 63.0 | 62.3 | 89.0 ± 5.9 |
| KC3-OA (46)/DSPC/DSPS | 101.9 | 110.0 | 94.1 ± 10.0 |
| KC3-OA(48)/DSPC/DSPS | 96.9 | 110.6 | 93.8 ± 16.8 |
| KC3-OA(50/DSPC/DSPS | 109.0 | 108.0 | 93.1 ± 14.4 |
| KC3-OA(52)/DSPC/DSPS | 108.6 | 110.9 | 93.8 ± 10.0 |
| KC3-OA(54)/DSPC/DSPS | 121.2 | 122.8 | 93.8 ± 17.0 |

All DSPS-targeted KC3-OA formulations showed a particle size around 100-120 nm and greater than 90% encapsulation efficiency. All particles were stable to freezing and thawing at −80° C. There was no apparent impact of the concentration of KC3-OA used in the range of 46-54 mol % on these properties.

TABLE 23
mCherry expression in human dendritic cells following
incubation at 1 μg/ml for DSPS-targeted KC3-OA
LNPs containing various concentrations of KC3-OA

| | | vs | vs |
|---|---|---|---|
| | | SM-102 | ALC-0315 |
| LNP formulation | MFI | (fold) | (fold) |
| Untreated (UT) | 1,343 ± 104 | | |
| SM-102/DSPC | 4,242 ± 1,942 | | |
| ALC-0315/DSPC | 3,760 ± 1,625 | | |
| KC3-OA (46)/DSPC/DSPS | 100,521 ± 44,930 | 34 | 41 |
| KC3-OA(48)/DSPC/DSPS | 116,994 ± 52,141 | 40 | 48 |
| KC3-OA(50/DSPC/DSPS | 107,911 ± 46,614 | 37 | 44 |
| KC3-OA(52)/DSPC/DSPS | 67,845 ± 37,779 | 23 | 28 |
| KC3-OA(54/DSPC/DSPS | 101,338 ± 46,945 | 34 | 41 |

TABLE 24
mCherry expression in human dendritic cells following
incubation at 0.1 μg/ml for DSPS-targeted KC3-OA
LNPs containing various concentrations of KC3-OA

| | | vs | vs |
|---|---|---|---|
| | | SM-102 | ALC-0315 |
| LNP formulation | MFI | (fold) | (fold) |
| Untreated (UT) | 1,295 ± 116 | | |
| SM-102/DSPC | 1,492 ± 26 | | |
| ALC-0315/DSPC | 1,405 ± 28 | | |
| KC3-OA (46)/DSPC/DSPS | 6,915 ± 1,659 | 29 | 51 |
| KC3-OA(48)/DSPC/DSPS | 10,491 ± 3,164 | 47 | 84 |
| KC3-OA(50/DSPC/DSPS | 11,033 ± 3,043 | 49 | 89 |
| KC3-OA(52)/DSPC/DSPS | 8,393 ± 2,447 | 36 | 65 |
| KC3-OA(54)/DSPC/DSPS | 7,496 ± 1,855 | 31 | 57 |

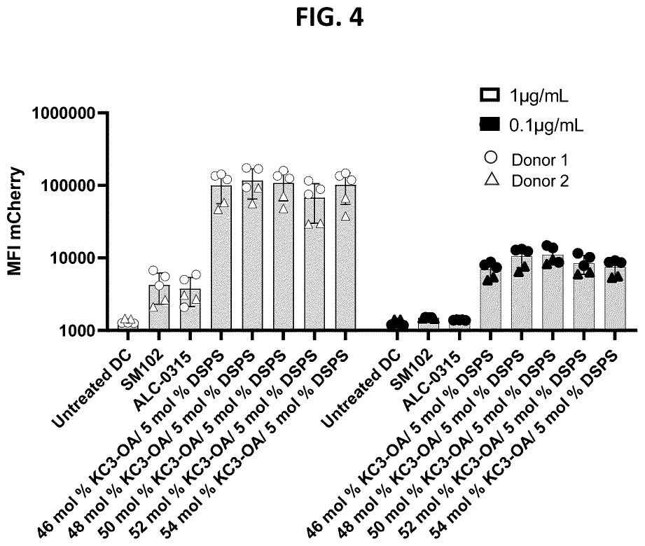

The effect of targeting and ICL choice on transfection activity was evaluated using 5 mol % DSPS-targeted LNPs comprised of KC3-OA at concentrations ranging from 46-54 mol % (FIG. 4). This targeted composition was highly active at all concentrations of KC3-OA at both 0.1 and 1 μg/mL, and only showed a slight peak at 48-50 mol % KC3-OA. However, all variations of this DSPS-targeted KC3-OA LNP formulation were significantly more active than either SM-102/DSPC or ALC-0315/DSPC controls (Tables 34-36).

### Example 11. Impact of N/P Ratio in PS-Targeted LNPs on Expression in Human Dendritic Cells

The aim of this study was to explore the effect of varying the N/P ratio in DSPS-targeted KC2-01 LNPs on transfection efficiency in human dendritic cells. The N/P ratio was varied from 4-7, while keeping the DSPS and DSPC concentrations constant at 5 mol % each, the concentration of KC2-01 constant at 50 mol %, and PEG-DMG at 1.5 mol %. ALC-0315 and SM-102 comparator formulations were also evaluated. The final ALC-0315/DSPC formulation was composed of 46.3 mol % ALC-0315, 9.4 mol 0 DSPC, 42.7 mol cholesterol and 1.56 mol % PEG-DMG with a N/P of 6.2. The final SM-102/DSPC formulation was composed of 50 mol % SM-102, 10 mol 00 DSPC, 38.5 mol 0 cholesterol and 1.5 mol %. PEG-DMG with a N/P of 5.

LNPs were prepared as described in Example 2A, characterized for particle size and zeta potential as described in Example 3, and evaluated for transfection efficiency in human dendritic cells as described in Example 6.

TABLE 25
Physicochemical properties and characterization of DSPS-targeted
KC2-O1 LNPs containing KC2-O1 at various N/P ratios

| | | Particle Size | |
|---|---|---|---|
| | Particle | (nm) Post- | Encapsulation |
| LNP formulation | size | Freeze/Thaw | Efficiency (%) |
| KC2-O1 (N/P = 4) | 85.4 | 91.3 | 83.2 ± 4.7 |
| KC2-O1/DSPS (N/P = 4) | 84.1 | 90.2 | 85.5 ± 3.9 |
| KC2-O1 (N/P = 5) | 84.9 | 87.0 | 88.0 ± 4.7 |
| KC2-O1/DSPS (N/P = 5) | 80.91 | 88.0 | 88.3 ± 4.0 |
| KC2-O1 (N/P = 6) | 81.1 | 84.0 | 88.0 ± 1.3 |
| KC2-O1/DSPS (N/P = 6) | 82.2 | 88.0 | 89.6 ± 2.7 |
| KC2-O1 (N/P = 7) | 75.7 | 84.0 | 89.0 ± 3.1 |
| KC2-O1/DSPS (N/P = 7) | 79.5 | 81.5 | 88.8 ± 4.1 |

All DSPS-targeted KC2-O1 formulations showed a particle size around 76-85 nm and greater than 80% encapsulation efficiency. All particles were stable to freezing and thawing at −80° C. There was no apparent impact of the N/P used in the range of 4-7 on these properties.

TABLE 26
mCherry expression in human dendritic cells following
incubation at 0.1 μg/ml for DSPS-targeted KC2-O1
LNPs containing KC2-O1 at various N/P ratios

| LNP formulation | MFI | T/NT | vs N/P = 4 |
|---|---|---|---|
| Untreated (UT) | 1,245 ± 3 | | |
| KC2-O1 (N/P = 4) | 2,077 ± 40 | | |
| KC2-O1/DSPS (N/P = 4) | 12,466 ± 2,359 | 13.5 | 1 |
| KC2-O1 (N/P = 5) | 1,966 ± 163 | | |
| KC2-O1/DSPS (N/P = 5) | 35,574 ± 13,780 | 47.6 | 3.1 |
| KC2-O1 (N/P = 6) | 3,389 ± 1,157 | | |
| KC2-O1/DSPS (N/P = 6) | 29,617 ± 3,345 | 13.2 | 2.5 |
| KC2-O1 (N/P = 7) | 5,852 ± 1,961 | | |
| KC2-O1/DSPS (N/P = 7) | 39,097 ± 6,398 | 8.2 | 3.4 |

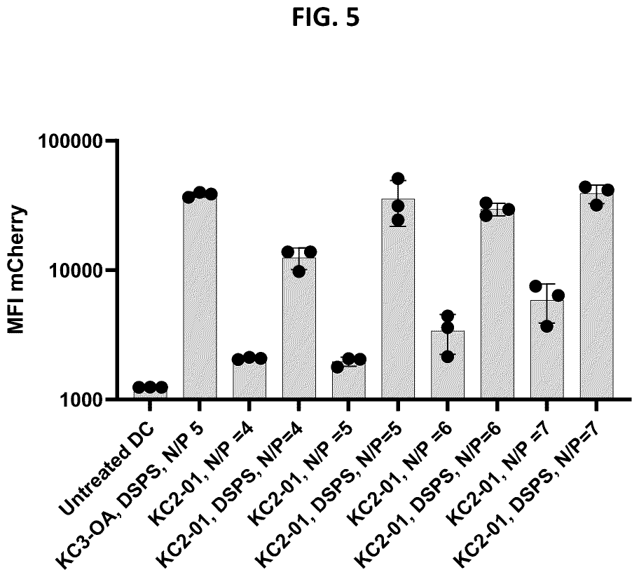

The effect of targeting and N/P ratio on transfection activity was evaluated using 5 mol % DSPS-targeted LNPs comprised of KC2-01 at ratios of 4-7 (FIG. 5). This targeted composition was highly active at all N/P ratios, but did display an approximately 3-fold drop off going from N/P of 5 to N/P of 4. The targeted to nontargeted (T/NT) ratio displayed a clear peak at N/P=5 (Table 26).

### Example 12. Preparation of Ammonium Salts of DPPS and DSPS

Dissolve 1.5 g PS sodium salt (DPPS-Na or DSPS-Na) in 500 ml of CHCl₃:Methanol:Water (CMW) 1:3:1 by volume. with 1M NH₄Cl in the water, to make a single-phase solution in a separatory funnel. Swirl 3-5 minutes, apply minimal heat if PS is not completely dissolved. Add CHCl3 and distilled water (approximately 200 mL each) and shake the funnel 2-3 minutes until a clear two-phase partition is achieved. Collect the bottom layer and re-partition with CMW 1:1:1 (by volume) with 1M NH₄Cl in the water. Collect the bottom layer and wash it 2× with fresh methanol-water (the volume of methanol is 50% of the volume of CHCl₃ and the volume of water is 20% of the total volume of chloroform and methanol) until a clear two-phase partition is achieved. Remove solvents under vacuum and freeze-dry from cyclohexane. Yield 1.2 g.

### Example 13. Apparent Solubility of Phosphatidylserine Salts in Ethanol

Apparent solubilities of phosphatidylserine salts in ethanol were determined by shake flask technique. Sodium and ammonium salts of phosphatidylserines in the powder form were obtained from Avanti Polar Lipids (Alabama, USA). Aliquots of phosphatidylserine salt powder were placed in 12×75 borosilicate glass tubes in triplicate. Three mL of 200 proof ethanol (catalog number E-7023, MilliporeSigma, USA) were added, the tubes were closed with polyethylene snap caps, agitated using vortex mixer for 10-15 s to obtain uniform suspensions, and placed in a horizontal position on a rocking platform at room temperature (20-22° C.) overnight. The ambient temperature at the end of incubation was 20.4-20.9° C. After 24 hours incubation, the suspensions were visually checked for the presence of a solid phase, allowed to settle under gravity, and the supernatant solutions were passed through a 0.2-μm PTFE syringe membrane filters. The first 0.5 ml of the filtrate was discarded, the next 1 ml was collected, and phosphatidylserine in the filtrates was quantified by phospholipid phosphate assay as follows: 20 μL aliquots of the filtrate were taken in triplicate into glass tubes, ethanol was evaporated in a stream of argon at 70° C., and the residue was digested in a mixture of 60 μL concentrated sulfuric acid and 20 μL 30% hydrogen peroxide for 10 min at 180-190° C. The digested samples were diluted with 1 mL of deionized water, 20 μL of 10% sodium sulfite was added to destroy any residual peroxide, and the samples were incubated on a boiling water bath for 15 min to hydrolyze any condensed phosphate species. The samples were chilled down to room temperature, mixed with 0.2 mL of 2% (w/w) ammonium molybdate solution and 20 μL of 10% (w/w) ascorbic acid solution, incubated on a boiling water bath for 10 min, and chilled down in a water bath at room temperature. The phosphate concentration was determined from the optical density of the formed blue phosphomolybdic acid at 825 nm using five-point standard curve from concurrently run standards prepared from the NIST-traceable commercial phosphate standard solution diluted to cover the range of 0-2 mM phosphate (coefficient of determination R² >0.9999). Solubility of phosphatidylserine salts was expressed in molarity units of phosphatidylserine (Table 40). The data are average±SD (N=3).

TABLE 27
Solubility of phosphatidylserine salts
in 100% ethanol at room temperature.

| Phosphatidylserine salt | Solubility, mM phosphatidylserine |
|---|---|
| DSPS sodium | 0.111 ± 0.009 |
| DPPS sodium | 0.201 ± 0.004 |
| DSPS ammonium | 0.034 ± 0.004 |
| DPPS ammonium | 0.865 ± 0.060 |

Ammonium salt of dipalmitoylphosphatidylserine (DPPS) unexpectedly showed several-fold higher ethanol solubility than ammonium salt of distearoylphosphatidylserine (DSPS) or sodium salts of any of these compounds.

### Example 14. Targeting of Human Dendritic Cells with Phosphatidylglycerol

The aim of this study was to explore the effect of targeting LNPs containing UO-1 or KC3-01 PCLs with various densities of distearoylphosphatidylglycerol (DSPG) on transfection efficiency in human dendritic cells. DSPG was incorporated in the LNPs at densities ranging from 0-10 mol % of the total lipid content. Control LNPs included KC3-OA LNPs with the previously established 5 mol % of DSPS as the targeting lipid, and nontargeted SM-102 or ALC-0315 containing LNPs. LNPs were prepared as described in Example 2A, characterized for particle size and zeta potential as described in Example 3, and evaluated for transfection efficiency in human dendritic cells as described in Example 6. The LNPs used the ionizable lipids in Table 45 in the ICL with a constant N/P ratio of 5.25 and 48 mol 00, the DSPG (Na⁺ salt) lipid was included at 0-mol 0 and the DSPC phospholipid constant at 10 mol 84. mol 1 incorporated of DSPG (total of DSPG and DSPC was 10 mol %), and the cholesterol constant at 40.5 mol % PEG-DMG was held constant in all formulations at 1.5 mol % The final ALC-0315/DSPC formulation was composed of 46.3 mol 0 ALC-0315, 10 mol-DSPC, 42.7 mol cholesterol and 1.5 mol PEG-DMG. The SM-102/DSPC formulation was composed of 50 mol 0 SM12, 10 mol 0 DSPC, 38.5 mol 0 cholesterol and 1.5 mol 0 PEG-DMG.

TABLE 28
Physicochemical properties and characterization
of LNPs used in Example 14

| | | Zeta | Zeta | Encap- |
|---|---|---|---|---|
| | Particle | Potential | Potential | sulation |
| | size | (mV) | (mV) | Efficiency |
| LNP formulation | (nm) | pH 5 | pH 7 | (%) |
| KC3-OA, 5% DSPS | 85.2 | 13.9 | −1.9 | 90.3 ± 6.4 |
| SM102 | 71.0 | 6.0 | −3.5 | 84.1 ± 10.7 |
| ALC-0315 | 63.0 | 5.2 | −5.2 | 89.0 ± 5.9 |
| UO1 | 99.2 | 25.4 | 2.9 | 86.9 ± 2.5 |
| UO1, 1.25% DSPG | 101.2 | 17.7 | 0.4 | 85.3 ± 2.4 |
| UO1, 2.5% DSPG | 89.7 | 13.5 | −2.3 | 83.8 ± 8.4 |
| UO1, 5.0% DSPG | 81.2 | 17.3 | −0.7 | 90.9 ± 2.7 |
| UO1, 7.5% DSPG | 65.9 | 15.9 | −1.8 | 91.0 ± 8.1 |
| UO1, 10% DSPG | 67.7 | 16.7 | −2.9 | 90.5 ± 6.6 |
| KC3-01 | 95.0 | 26.4 | 1.5 | 90.3 ± 11.5 |
| KC3-01, 1.25% DSPG | 98.4 | 27.4 | 0.0 | 90.7 ± 3.1 |
| KC3-01, 2.5% DSPG | 82.1 | 18.5 | −0.8 | 90.6 ± 3.2 |
| KC3-01, 5.0% DSPG | 72.4 | 18.1 | −2.1 | 89.9 ± 3.8 |
| KC3-01, 7.5% DSPG | 71.0 | 14.8 | −2.2 | 89.4 ± 3.6 |
| KC3-01, 10% DSPG | 74.6 | 13.0 | −2.3 | 89.5 ± 13.5 |

TABLE 29
mCherry expression in human dendritic
cells following incubation at 1 μg/ml

| | | | vs | vs |
|---|---|---|---|---|
| | | | ALC-0315 | SM-102 |
| | | | LNP | LNP |
| LNP formulation | MFI | T/NT | (fold) | (fold) |
| Untreated (UT) | 1,453 ± 263 | | | |
| KC3-OA, 5% DSPS | 89,815 ± 16,603 | | 76.8 | 8.4 |
| SM102 | 11,924 ± 1,089 | | | |
| ALC-0315 | 2,604 ± 376 | | | |
| UO1 | 2,049 ± 297 | | 0.5 | 0.1 |
| UO1, 1.25% DSPG | 2,728 ± 950 | 2.1 | 1.1 | 0.1 |
| UO1, 2.5% DSPG | 8,134 ± 3,763 | 11.2 | 5.8 | 0.6 |
| UO1, 5.0% DSPG | 22,206 ± 3,488 | 34.8 | 18.0 | 2.0 |
| UO1, 7.5% DSPG | 12,424 ± 1,939 | 18.4 | 9.5 | 1.0 |
| UO1, 10% DSPG | 5,561 ± 639 | 6.9 | 3.6 | 0.4 |
| KC3-01 | 49,931 ± 14,344 | | 42.1 | 4.6 |
| KC3-01, 1.25% DSPG | 119,467 ± 21,513 | 2.4 | 102.6 | 11.3 |
| KC3-01, 2.5% DSPG | 112,191 ± 12,156 | 2.3 | 96.2 | 10.6 |
| KC3-01, 5.0% DSPG | 86,696 ± 10,231 | 1.8 | 74.1 | 8.1 |
| KC3-01, 7.5% DSPG | 25,230 ± 2,061 | 0.5 | 20.7 | 2.3 |
| KC3-01, 10% DSPG | 8,250 ± 2,442 | 0.1 | 5.9 | 0.6 |

TABLE 30
mCherry expression in human dendritic
cells following incubation at 0.1 μg/ml

| | | | vs | vs |
|---|---|---|---|---|
| | | | ALC-0315 | SM-102 |
| | | T/NT | LNP | LNP |
| LNP formulation | MFI | (fold) | (fold) | (fold) |
| Untreated (UT) | 1,403 ± 190 | | | |
| KC3-OA, 5% DSPS | 10,347 ± 3,118 | | 308.6 | 48.0 |
| SM102 | 1,590 ± 243 | | | |
| ALC-0315 | 1,432 ± 198 | | | |
| UO1 | 1,393 ± 237 | | | |
| UO1, 1.25% DSPG | 1,409 ± 262 | 0.5 | 0.2 | 0 |
| UO1, 2.5% DSPG | 1,474 ± 280 | 6.7 | 2.4 | 0.4 |
| UO1, 5.0% DSPG | 2,253 ± 1,089 | 81.0 | 29.3 | 4.6 |
| UO1, 7.5% DSPG | 1,887 ± 400 | 46.2 | 16.7 | 2.6 |
| UO1, 10% DSPG | 1,515 ± 252 | 10.7 | 3.9 | 0.6 |
| KC3-01 | 1,976 ± 112 | | 19.8 | 3.1 |
| KC3-01, 1.25% DSPG | 2,377 ± 329 | 1.7 | 33.6 | 5.2 |
| KC3-01, 2.5% DSPG | 3,801 ± 485 | 4.2 | 82.7 | 12.9 |
| KC3-01, 5.0% DSPG | 8,580 ± 786 | 12.5 | 247.6 | 38.5 |
| KC3-01, 7.5% DSPG | 3,075 ± 279 | 2.9 | 57.7 | 9.0 |
| KC3-01, 10% DSPG | 1,619 ± 146 | 0.4 | 7.4 | 1.2 |

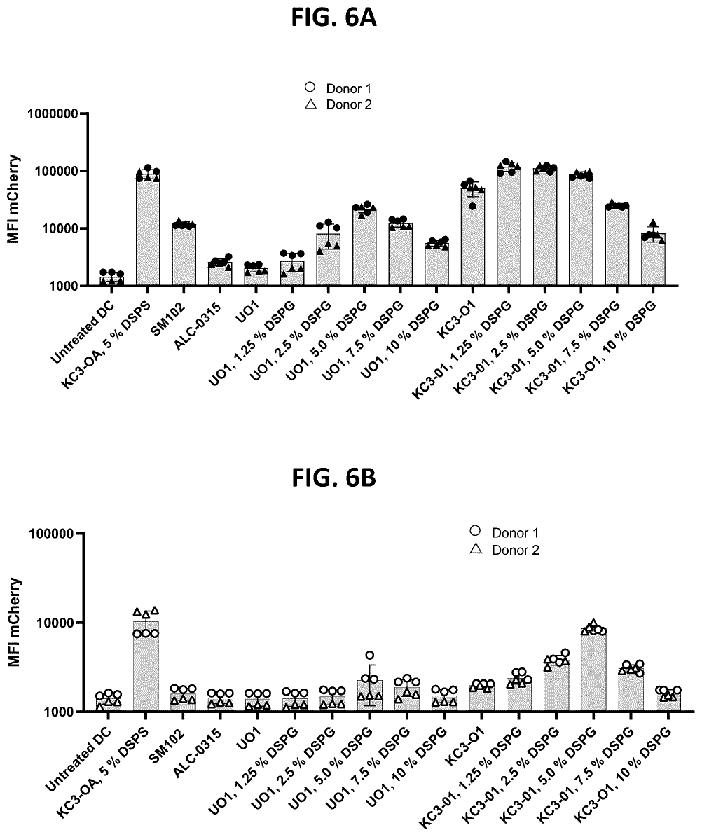

The effect of targeting two different LNPs with DSPG on transfection activity was evaluated using 0-10 mol % DSPG-targeted LNPs (FIGS. 6A-6B). For UO-1 containing LNPs the optimum DSPG concentration was 5 mol % at both 1 and 0.1 ug/ml, resulting in a 34.8 folding targeting effect at 1 μg/ml and 81-fold improvement over nontargeted LNPs at 0.1 ug/ml. For KC3-01 containing LNPs the optimum DSPG concentration was also at 5 mol % for the 0.1 ug/mL, but showed a broader peak of 1.25-5 mol % at the higher concentration of 1 μg/mL with a targeting effect of 2.4-fold at 1 μg/mL and 12.5-fold at the lower concentration of 0.1 ug/mL. Both of the peak targeted LNPs showed increased transfection activity compared to nontargeted ALC-0315 and SM-102 LNPs (Tables 46 and 47), with an up to 102.6% increase compared to ALC-0315 for DSPG-targeted KC3-01 LNPs at 1 μg/mL and an up to 247.6 fold increase at 0.1 ug/mL. The improvement over SM-102 LNPs was as high as 38.50-fold for the 5 mol % DSPG targeted KC3-01 LNP and 48-fold for the DSPS-targeted targeted KC3-OA LNP control. This data shows that DSPG can be a potent targeting anionic lipid to increase transfection efficiency in human dendritic cells.

### Example 15. Comparison of DSPC and DPPC as Helper Lipids in a KC3-PA-Based LNP Formulation

The aim of this study was to explore comparing DSPC and DPPC in KC3-PA DPPS-targeted and non-targeted LNPs. The concentration of KC3-OA was 48 mol %, cholesterol 40.5 mol %, PEG-DMG 1.5 mol %. The phospholipid composition was kept constant at 10 mol %, but for those that were targeted with DPPS, the DPPS concentration was 5 mol % and either DSPC or DPPC at 5 mol %. All formulations had an N/P of 5.25.

LNPs were prepared as described in Example 2A, and were characterized for particle size, mRNA concentration and % mRNA entrapment after ethanol removal by dialysis and after sterile filtration as described in Example 3.

TABLE 31
Physicochemical properties and characterization
of KC3-PA-targeted and non-targeted LNPs

| | Particle Size | Encapsulation | % mRNA |
|---|---|---|---|
| | (nm) after | Efficiency | Loss upon |
| LNP formulation | filtration | (%) | filtration |
| KC3-PA/DPPC | 108.5 | 82.2 ± 2.4 | 35.5 |
| KC3-PA/DPPC/DPPS-NH⁴ | 107.7 | 90.6 ± 6.1 | 10.6 |
| KC3-PA/DSPC | 114.0 | 86.4 ± 5.7 | 12.5 |
| KC3-PA/DSPC/DPPS-NH⁴ | 91.6 | 91.3 ± 6.7 | 4.4 |

Once the LNPs have been prepared and the ethanol removed by dialysis, the measurement of mRNA concentration before and after filtration provides another metric to evaluate the formulations. It is advantageous if LNPs have a high efficiency of sterile filtration, where mRNA loss is minimized and eliminates membrane fouling and poor product yields. In the non-targeted group, we found a 2.8-fold improvement in mRNA recovery with the DSPC sample compared to DPPC and in the targeted group we found a 2.4-fold increase in mRNA recovery. This suggests that DSPC is a more compatible helper lipid in formulations with KC3-PA than DPPC with and without 5 mol % DPPS.

### Example 16. Impact of PG and PS Targeting of KC3-OA Containing LNPs on mRNA Expression in Human Dendritic Cells

The aim of this study was to explore the effect of targeting LNPs containing KC3-OA ICLs with various forms of phosphatidylglycerol (PG), including distearoylphosphatidylglycerol (DSPG), dipalmitoylphosphatidylglycerol (DPPG), dioleoylphosphatidylglycerol (DOPG), and dimyristoylphosphatidylglycerol (DMPG) on transfection efficiency in human dendritic cells. A second aim was to compare these targeted LNPs to those containing DSPS, and finally to compare formulations that incorporated two anionic phospholipids (DSPG and DSPS).

PG in various forms was incorporated in the LNPs at densities ranging from 0-5 mol % of the total lipid content. Control LNPs included KC3-OA LNPs with the previously established 5 mol % of DSPS as the targeting lipid. LNPs were prepared as described in Example 2A, characterized for particle size and zeta potential as described in Example 3, and evaluated for transfection efficiency in human dendritic cells as described in Example 6. The LNPs used the ionizable lipids in Table 52 in the ICL with a constant N/P ratio of 5.25 and 48 mol %, the DSPG (Na⁺ salt) lipid was included at 0-10 mol % and the DSPC phospholipid was also varied from 0-mol %, and the cholesterol constant at 40.5 mol %. Some formulations included DSPS and DSPG, with or without DSPC. PEG-DMG was held constant in all formulations at 1.5 mol %.

TABLE 32
Physicochemical properties and characterization
of LNPs used in Example

| | | Zeta | Zeta | Encap- |
|---|---|---|---|---|
| | Particle | Potential | Potential | sulation |
| | size | (mV) | (mV) | Efficiency |
| LNP formulation | (nm) | pH 5 | pH 7 | (%) |
| 10% DSPC | 91.9 | 22.19 | 1.712 | 92.7 ± 3.1 |
| 5% DSPG/5% DSPC | 94.0 | 15.01 | −6.99 | 90.3 ± 2.1 |
| 5% DOPG/5% DSPC | 77.0 | 15.64 | 2.303 | 92.0 ± 7.5 |
| 5% DPPG/5% DSPC | 93.6 | 14.7 | −0.2016 | 90.6 ± 2.7 |
| 5% DMPG/5% DSPC | 66.0 | 19.29 | 0.8961 | 92.8 ± 8.0 |
| 5% DSPS/5% DSPC | 87.9 | 23.18 | −1.219 | 92.7 ± 4.5 |
| 5% DSPG/5% DSPS | 69.9 | 25.61 | −1.651 | 93.3 ± 6.1 |
| 2.5% DSPG/5% DSPS/ | 72.2 | 19.36 | −1.596 | 92.7 ± 6.0 |
| 2.5% DSPC | | | | |
| 5% DSPG/5% DSPS/ | 66.6 | 16.72 | −5.128 | 92.6 ± 2.0 |
| 2.5% DSPC | | | | |
| 5% DSPG/5% DSPS/ | 69.1 | 17.75 | −4.506 | 93.5 ± 3.3 |
| 5% DSPC | | | | |
| 5% DSPG/5% DSPS/ | 66.9 | 11.11 | −5.389 | 93.4 ± 9.0 |
| 7.5% DSPC | | | | |
| 5% DSPG/5% DSPS/ | 64.6 | 15.43 | −6.708 | 93.2 ± 4.2 |
| 10% DSPC | | | | |

All formulations showed particle sizes between 65-100 nm with a slightly negative charge at pH 7.4 and a clearly positive zeta potential of 10-25 mV at pH 5. All particles had greater than 90% encapsulation efficiency, despite the presence of anionic phospholipids that could compete for binding with the negative charged phosphates on mRNA.

TABLE 33
mCherry expression in human dendritic cells following incubation at 1 μg/ml

| | MFI | T/NT | MFI | T/NT |
|---|---|---|---|---|
| LNP formulation | (donor 1) | (donor 1) | (donor 2) | (donor 2) |
| Untreated (UT) | 2,735 ± 56 | | 1,354 ± 19 | |
| 10% DSPC | 14,152 ± 661 | | 14,034 ± 5,257 | |
| 5% DSPG/5% DSPC | 43,413 ± 5,029 | 3.56 | 53,670 ± 1,974 | 4.13 |
| 5% DOPG/5% DSPC | 16,783 ± 1,375 | 1.23 | 62,404 ± 2,841 | 4.81 |
| 5% DPPG/5% DSPC | 39,042 ± 6,957 | 3.18 | 58,557 ± 3,456 | 4.51 |
| 5% DMPG/5% DSPC | 70,516 ± 1,584 | 1.79 | 51,862 ± 5,953 | 3.98 |
| 5% DSPS/5% DSPC | 70,516 ± 1,584 | 5.94 | 225,546 ± 11,751 | 17.68 |
| 5% DSPG/5% DSPS | 10,148 ± 313 | 0.65 | 15,158 ± 289 | 1.09 |
| 2.5% DSPG/5% DSPS/ | 35,797 ± 2,364 | 2.90 | 85,066 ± 8,576 | 6.60 |
| 2.5% DSPC | | | | |
| 5% DSPG/5% DSPS/ | 27,349 ± 4,520 | 2.16 | 59,403 ± 4,237 | 4.58 |
| 2.5% DSPC | | | | |
| 5% DSPG/5% DSPS/ | 52,500 ± 1,351 | 4.36 | 98,242 ± 1,421 | 7.64 |
| 5% DSPC | | | | |
| 5% DSPG/5% DSPS/ | 49,439 ± 3,660 | 4.09 | 80,055 ± 7,036 | 6.21 |
| 7.5% DSPC | | | | |
| 5% DSPG/5% DSPS/ | 32,566 ± 4,525 | 2.61 | 47,190 ± 9,456 | 3.61 |
| 10% DSPC | | | | |

TABLE 34
mCherry expression in human dendritic cells following incubation at 0.1 μg/ml

| | MFI | T/NT | MFI | T/NT |
|---|---|---|---|---|
| LNP formulation | (donor 1) | (donor 1) | (donor 2) | (donor 2) |
| Untreated (UT) | 2,687 ± 27 | | 1,390 ± 11 | |
| 10% DSPC | 3,330 ± 216 | | 1,721 ± 36 | |
| 5% DSPG/5% DSPC | 6,292 ± 155 | 5.61 | 4,289 ± 19 | 8.76 |
| 5% DOPG/5% DSPC | 4,288 ± 52 | 2.49 | 5,951 ± 370 | 13.78 |
| 5% DPPG/5% DSPC | 6,754 ± 312 | 6.32 | 5,243 ± 535 | 11.64 |
| 5% DMPG/5% DSPC | 4,912 ± 647 | 3.46 | 4,202 ± 199 | 8.49 |
| 5% DSPS/5% DSPC | 10,776 ± 161 | 12.58 | 21,283 ± 1,303 | 60.10 |
| 5% DSPG/5% DSPS | 3,379 ± 112 | 1.08 | 3,574 ± 178 | 6.60 |
| 2.5% DSPG/5% DSPS/ | 6,190 ± 184 | 5.45 | 11,379 ± 133 | 30.18 |
| 2.5% DSPC | | | | |
| 5% DSPG/5% DSPS/ | 4,967 ± 291 | 3.55 | 6,028 ± 65 | 14.01 |
| 2.5% DSPC | | | | |
| 5% DSPG/5% DSPS/ | 8,353 ± 153 | 8.81 | 9,758 ± 214 | 25.28 |
| 5% DSPC | | | | |
| 5% DSPG/5% DSPS/ | 10,604 ± 126 | 12.31 | 10,602 ± 1,620 | 27.83 |
| 7.5% DSPC | | | | |
| 5% DSPG/5% DSPS/ | 11,761 ± 502 | 14.11 | 10,139 ± 339 | 26.43 |
| 10% DSPC | | | | |

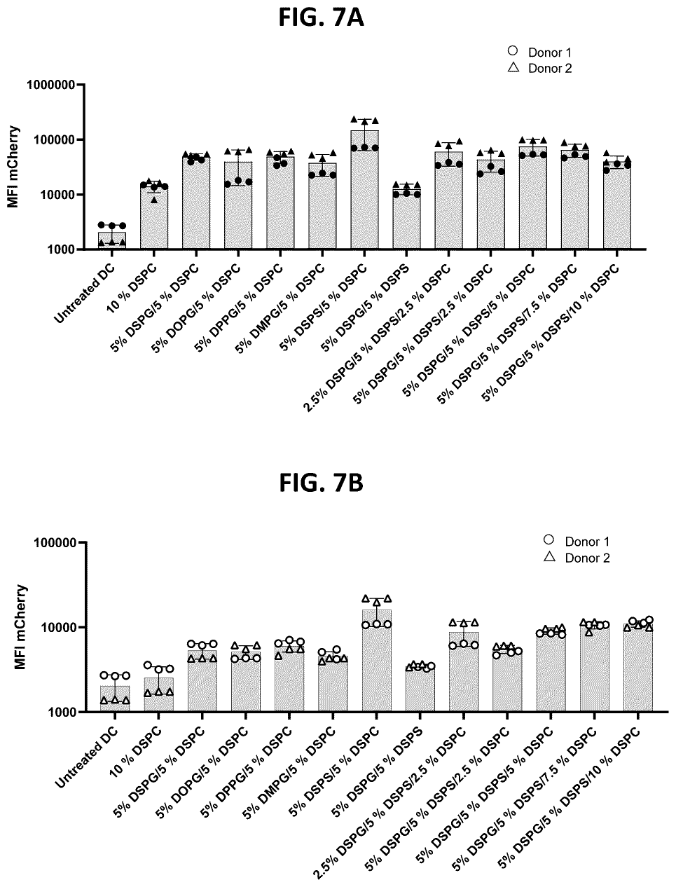

The effect of targeting two different LNPs with DSPG on transfection activity was evaluated using PG-targeted LNPs of various compositions (FIGS. 7A-7B). For KC3-OA containing LNPs, all forms of PG showed some level of increased expression relatively to 10% DSPC controls, and although there was small preference for the saturated DPPG or DSPG forms of PG with one donor, in a separate donor DOPG and DMPG showed similar activity. The highest increase over DSPC control came from the 5% DSPS targeted LNP, and combining DSPS with DSPG did not improve transfection activity further, and in most instances was antagonistic when compared to those formulations containing DSPS as the sole anionic phospholipid. In fact, those LNP formulations where all the phospholipids were either DSPG or DSPS (e.g. no DSPC) showed the lowest transfection activity of the targeted LNP formulations. This data shows that both PG and DSPS can be potent targeting anionic lipids to increase transfection efficiency in human dendritic cells, although combining the two does not appear to further improve activity.

### Example 17. Use of Ethanol Soluble DPPS-NH4 as Opposed to the Less Soluble DSPS-Na Allows for Room Temperature Preparation of Phosphatidylserine-Targeted KC3OA-Based LNPs

The aim of this study was to compare DSPS-Na and DPPS-NH₄ as targeting ligands in LNPs by measuring their transfection efficiency in murine DC cells. LNPs were prepared as in Example 3. Prior to LNP preparation stock solutions of each lipid were made in ethanol before use, except DSPS-Na, which because of its low solubility in ethanol (see Example 15) was dissolved in methanol at elevated temperatures (70° C.) to ensure complete dissolution. Once cooled to room temperature, the methanol solution will maintain DSPS-Na solubility for a short period of time (<1 hour). Therefore, after addition of KC3OA, DSPC, Chol and PEG-DMG and DSPS-Na from their respective lipid stocks, the resulting mixture contains about 16% methanol by volume in ethanol. To make DSPS-targeted LNPs, and to ensure complete solubility of DSPS-Na, the ethanol/methanol solution is incubated at 70 C in a syringe holder heating block prior to mixing with pre-warmed mRNA. Care must be taken to maintain elevated temperature control prior to LNP formation, otherwise DSPS aggregation or LNP aggregation can occur. Once the LNPs were formed, the suspension was allowed to cool naturally to room temperature before solvent removal by dialysis. KC3OA was kept constant at 48 mol %, the DSPC concentration was mol % with either 5 mol % DSPS-Na or DPPS-NH₄, 38.5 mol % cholesterol and 1.5 mol % PEG-DMG. The N/P was 5.25. The KC3OA/DSPS sample contained 1575 nmol KC3OA, 164.1 nmol DSPC, 164.1 nmol DSPS-Na, 1328.9 nmol cholesterol and 49.2 nmol PEG-DMG per 100 μg mRNA. In contrast, because of the higher solubility of DPPS-NH₄, LNPs can be prepared from all ethanol stock solutions, thereby eliminating the need for high temperature incubation before mixing and eliminates methanol from the process, which are both advantageous from a scale-up and residual solvent perspective. The KC3OA/DPPS sample contained 1575 nmol KC3OA, 164.1 nmol DSPC, 164.1 nmol DPPS-NH₄, 1328.9 nmol cholesterol and 49.2 nmol PEG-DMG per 100 μg mRNA and was mixed at room temperature. The final ALC-0315/DSPC formulation was composed of 46.3 mol % ALC-0315, 10 mol % DSPC, 42.7 mol % cholesterol and 1.5 mol % PEG-DMG. The SM-102/DSPC formulation was composed of 50 mol % SM102, 10 mol % DSPC, 38.5 mol % cholesterol and 1.5 mol % PEG-DMG, both controls prepared at room temperature.

LNPs were exchanged into PBS, pH 7.4 and then 15 mM Tris, pH 7.4, 20% sucrose, concentrated to 20-50 ug/mL mRNA using an Amicon-Ultra 4 (100,000 MWCO) spin column, sterile filtered (Thermo Nalgene 0.2 um #720-1320) prior to freezing by immersion in liquid nitrogen for 5 min and long-term storage at −80° C. LNPs were analyzed as described in Example 3, and were tested in murine DC cells as described in Example 4.

TABLE 35

| | | Zeta | Zeta | Encap- |
|---|---|---|---|---|
| | Particle | Potential | Potential | sulation |
| | Size | (mV) | (mV) | Efficiency |
| LNP formulation | (nm) | pH 5 | pH 7 | (%) |
| ALC-0315 | 71.0 | 6.0 | −3.5 | 84.1 ± 10.7 |
| SM102 | 63.0 | 5.2 | −5.2 | 89.0 ± 5.9 |
| KC3-OA/DSPS-Na | 83.9 | 19.04 | −0.8 | 92.6 ± 8.7 |
| KC3-OA/DPPS-NH₄ | 78.3 | 11.91 | −0.1 | 93.3 ± 1.2 |

TABLE 36

| | | vs | vs |
|---|---|---|---|
| | | ALC-0315 | SM-102 |
| LNP formulation | mCherry MFI | LNP (fold) | LNP (fold) |
| Untreated (UT) | 282.7 ± 3.8 | | |
| ALC-0315 | 1336.3 ± 91.6 | | |
| SM-102 | 6978.3 ± 1388 | | |
| KC3-OA/DSPS-Na | 16039.3 ± 4046.4 | 12.0 | 2.3 |
| KC3-OA/DPPS- NH₄ | 32326.3 ± 735.9 | 24.2 | 4.6 |

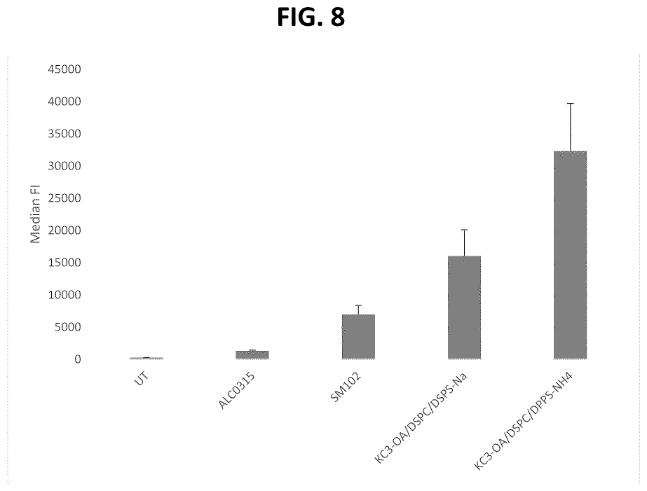

The KC3-OA/DPPS-NH₄ LNP was comparable to the DSPS-targeted LNP from a biophysical point of view, all measured characteristics were very similar. In addition, it was found that inclusion of DPPS induced an approximately 2-fold increase in mCherry expression (FIG. 8). The comparable biophysical LNP characteristics combined with the comparable and slightly improved mRNA expression, suggests that DPPS-NH₄ is an attractive PS-targeting ligand targeting for KC3-OA LNPs.

### Example 18. Impact of PL/Chol Ratio in PS-Targeted LNPs on Biophysical Characteristics and Transfection Efficiency in Murine DCs

The aim of this study was to explore the effect of varying the phospholipid/cholesterol ratio (PL/Chol ratio) in DPPS-targeted KC3-OA LNPs on biophysical characteristics and transfection efficiency in murine dendritic cells. The PL/Chol ratio was varied from 0.11-100 (5-50 mol % PL and 45.5-0.5 mol % Chol), while keeping the DPPS-NH₄, PEG-DMG and KC3-OA constant at 5 mol %, 1.5 mol %, and 48 mol % respectively with an N/P ratio of 5.25 using mCherry encoding mRNA. All mRNAs used N1-methylpseudouridine (N1MeU). An ALC-0315 based formulation composed of 46.3 mol % ALC-0315, 9.4 mol % DSPC, 42.7 mol % cholesterol and 1.56 mol % PEG-DMG with a N/P of 6.2 was also made and evaluated.

LNPs were prepared, characterized for particle size and zeta potential, mRNA encapsulation efficiency and mRNA concentration as described above and evaluated for transfection efficiency in murine dendritic cells as described in Example 4.

TABLE 37
Physicochemical properties and characterization of DPPS-targeted KC3-OA LNPs with varying
PL/Chol ratios, described in terms of PL/Chol mol %/mol % ratio. Particle size refers
to the Z-Average particle size. Post-F/T refers to samples that were measured after
a freeze/thaw event, and Zeta refers to zeta potential measurements at either pH 5 or
pH 7 in units of mV. mRNA encapsulation efficiency is referred to as (E.E, %).

| | PL/Chol | Particle size | Particle Size | | | |
|---|---|---|---|---|---|---|
| ICL | (mol %/mol %) | (nm) | (nm) Post-F/T | E.E, % | Zeta pH 5 | Zeta pH 7 |
| ALC-0315 | 9.4/42.7 | 94.00 | 93.14 | 81.0 ± 4.5 | 5.44 | −4.45 |
| KC3-OA | 5/45.5 | 116.30 | 114.40 | 91.6 ± 10.9 | 12.76 | −2.31 |
| KC3-OA | 10/40.5 | 109.50 | 110.30 | 91.6 ± 10.7 | 17.33 | −1.18 |
| KC3-OA | 15/35.5 | 99.02 | 97.60 | 92.7 ± 1.5 | 11.22 | −1.13 |
| KC3-OA | 20/30.5 | 82.74 | 81.50 | 91.9 ± 2.6 | 14.54 | −2.47 |
| KC3-OA | 25/25.5 | 80.92 | 79.80 | 91.5 ± 2.3 | 17.40 | −3.11 |
| KC3-OA | 30/20.5 | 82.85 | 83.48 | 90.7 ± 1.2 | 12.41 | 0.08 |
| KC3-OA | 35/15.5 | 96.60 | 95.90 | 90.1 ± 2.5 | 19.81 | −0.43 |
| KC3-OA | 40/10.5 | 117.4 | 118.6 | 85.9 ± 2.5 | 18.47 | −0.45 |
| KC3-OA | 45/5.5 | 113.7 | 116.1 | 85.8 ± 2.9 | 13.94 | 1.62 |
| KC3-OA | 50/0.5 | 96.25 | 97.27 | 83.0 ± 2.5 | 9.82 | 1.45 |

KC3-OA formulations showed a dependence of PL/Chol on particle size. The Z-average LNP particle size was highest (>100 nm) at the lowest and highest PL/Chol ratios tested, but at intermediate ratios (15/35.5-35.5/15 mol %/mol %) the size was found to be less than 100 nm and smallest at a PL ratio of 25/25.5 (mol %/mol %). At higher PL/Chol ratios (at 40 mol % PL or more) the E.E, % was reduced below 900 while >908 E.E, 4 was found with all KC3-OA LNPs with <40 mol % PL. All particles were stable to freezing and thawing at −80° C. as judged by the small changes in Z-Ave particle sizes after F/T.

TABLE 38
mCherry expression in murine dendritic cells MutuDC
1940 following incubation of mCherry containing mRNA-
LNPs at 0.1 μg/ml mRNA concentration for 18 h.

| | PL/Chol | |
|---|---|---|
| ICL | (mol %/mol %) | Med FI |
| Untreated cells | | 84.3 ± 3.7 |
| ALC-0315 | 9.4/42.7 | 1181.0 ± 80.5 |
| KC3-OA | 5/45.5 | 772.7 ± 431.4 |
| KC3-OA | 10/40.5 | 10476.7 ± 1749.9 |
| KC3-OA | 15/35.5 | 11377.3 ± 2361.1 |
| KC3-OA | 20/30.5 | 3799.7 ± 578.5 |
| KC3-OA | 25/25.5 | 1272.3 ± 97.9 |
| KC3-OA | 30/20.5 | 404.7 ± 32.5 |
| KC3-OA | 35/15.5 | 126.3 ± 4.2 |
| KC3-OA | 40/10.5 | 108.0 ± 2.6 |
| KC3-OA | 45/5.5 | 101.7 ± 4.6 |
| KC3-OA | 50/0.5 | 105.0 ± 2.6 |

MutuDC1940 cells were plated and LNPs added to cell culture media as described in Example 4. A striking dependence on in vitro mCherry activity to PL/Chol was observed. It was unexpectedly found that the transfection efficiency could be further optimized by increasing the total phospholipid content to 15 mol %. For example, the most mCherry expression was found with LNPs consisting of PL/Chol ratios of 10/40.5 and 15/35.5 which were both ˜100-fold higher than the 3 formulations tested with >40 mol % PL. In vitro mCherry expression varied with LNP particle size. For example, while the LNPs with 10/40.5, 15/35.5 PL/Chol had sizes ˜110 and 98 nm respectively and had the highest mCherry expression, LNPs with larger or smaller sizes displayed lower mCherry expression.

### Example 19. Preparation of Lipidic Nanoparticles

Synthetic CpG-oligodeoxynucleotide (ODN) 1018 (SEQ ID NO: 2) with phosphorothioate linkages was purchased from MedChemExpress (Cat. No. HY-150724). CpG-ODN 2006 (SEQ ID NO: 3) was purchased from InvivoGen (ODN 2006 VacciGrade, Cat. No. vac-2006-1). The TLR7/8 ssRNA oligonucleotide 5′-UUGUUGUUGUUGUUGUUGUU-3′ (SEQ ID NO: 25, referred to hereafter as “UUGU”) with phosphorothioate linkages was synthesized by AxoLabs and incorporated into LNPs at an N/P ratio of 5.25.

Stock solutions of each lipid were prepared. The ionizable lipid KC3-OA and cholesterol were weighed out in 4 mL glass vials (Thermo B7999-2) and dissolved in ethanol (Sigma-Aldrich 200 proof, RNase free) to a final concentration of 10 mM. Other lipids such as DSPC, DPPS-NH₄, and PEG-DMG were weighed out and dissolved in ethanol to a concentration of 1 mM. Lipid mixtures for each individual LNP were prepared by adding the desired volume of each lipid stock solution to a new vial, adding ethanol if needed to achieve a final volume of 1.2 mL. For example, an LNP formulation of KC3-OA/DSPC/DPPS/Chol/PEG-DMG (48/5/5/40.5/1.5 mol %), with an N/P of 5.25 contained 2362.5 nmol KC3-OA, 246.1 nmol DSPC, 246.1 nmol DPPS, 1993.4 nmol Chol and 73.8 nmol PEG-DMG for every 150 g of UUGU oligo used. UUGU oligo solutions were prepared by thawing frozen stock (1 mg/mL) vials and diluting in 6.25 mM sodium acetate (pH 5.0) to a final concentration of 0.05 mg/mL.

To prepare LNPs, a NanoAssemblr Benchtop microfluidic device (from Precision Nanosystems) was used. LNPs were mixed at room temperature. 3 mL of UUGU oligo solution was loaded into a 3 mL disposable syringe (BD 309656) and 1 mL of lipid mixture in a 1 ml syringe (BD309659) and syringes were inserted into the NanoAssemblr Benchtop microfluidics disposable cassette. LNP formation was achieved by pumping the liquid streams through the microfluidics cassette at 3:1 aqueous: alcohol volume ratio at 6 mL/min mixing speed. After mixing, 3.6 mL of LNP mixture was collected, while the initial mixed volume of 0.35 mL and last 0.05 mL of mix was discarded. Ethanol was removed by buffer exchange using Amicon Ultra-4 centrifugal concentrators (100k MWCO, at 500 g).

LNPs were exchanged into PBS, pH 7.4 and then 15 mM Tris, pH 7.4, 20% sucrose, concentrated to 20-50 ug/mL ssDNA UUGU oligo using an Amicon-Ultra 4 (100,000 MWCO) centrifugal concentrators, sterile filtered (Thermo Nalgene 0.2 um #720-1320) prior to freezing by immersion in liquid nitrogen for 5 min and long-term storage at −80° C.

### Example 20. LNP Characterization

Oligonucleotide encapsulation and concentration within the LNPs was measured using a Ribogreen assay. Nanoparticle size and zeta potential were measured by a zetasizer (Malvern). mRNA and DNA oligonucleotide encapsulation and concentration within the LNPs was measured using a Ribogreen or Oligreen assay respectively. Nanoparticle size and zeta potential were measured by a zetasizer (Malvern).

A. ssRNA Oligo (e.g. UUGU) Concentration and Relative Encapsulation Efficiency Determination by Fluorescent Binding Dye.

Materials: Quant-iT Ribogreen reagent (Thermo #11491), 96-well plates with lids, PBS, dissociation buffer (PBS, pH 7.4 with 10% DMSO and 1% (wt/wt) Zwittergent 3-14 (Sigma-Aldrich #693017), UUGU oligo, general pipette tips & repeater pipette tips. 1 mg/mL premade oligo stock (e.g. UUGU) was diluted 1:10 in PBS (100 μL oligo added to 900 μL PBS). Based on the UV absorption of this stock solution at 260 nm, the oligo concentration was estimated as equal 94.6 μg/mL using the absorbance characteristics of this oligo provided by the vendor of 1 O.D=35 μg/mL. A second dilution to 8 μg/mL was made in PBS, pH 7.4. From the stock solution at 8 μg/mL, diluted standards were prepared as follows in single wells in a deep 96-well plate.

TABLE 39

| Final [oligo] μg/mL | Vol. stock 8 μg/mL (μL) | Vol. PBS (μL) |
|---|---|---|
| 8 | 500 | 0 |
| 6 | 375 | 125 |
| 4 | 250 | 250 |
| 2 | 125 | 375 |
| 1 | 62.5 | 437.5 |
| 0 | 0 | 500 |

RNA oligo LNP samples were diluted prior to measurement so that the oligo concentrations would be within the selected standard curve. Two 96 well plates (A and B) were used for the assay. On each plate, 80 μL of each standard concentration were added into 2 wells each (duplicate), and 80 μL of diluted sample into 3 wells each (triplicate). 80 μl of Ribogreen diluted in PBS (diluted 1:100) to each well on plate A and 80 μL Ribogreen diluted 1:100 in Dissociation Buffer was added to plate B. Both plates A and B were mixed on an orbital mixer (120 rpm) for 1 min. Plate A was placed in the dark for 15 min. Plate B was incubated at 37° C. in the dark for 10 min, followed by 5 min at RT. Both plates were read one after the other, using Ex. 485, Em. 530 nm. Using the standard curve, the slope and intercept were calculated and by extrapolation the UUGU oligo concentrations of the samples on plate A & B were calculated (average and std dev). Percent encapsulation efficiency (% EE) was calculated as follows: RNA oligo concentration on plate A/RNA oligo concentration on plate B X 100. Total RNA oligo concentration was calculated by taking the RNA oligo concentration on plate B and multiplying by the dilution factor.

B. ssDNA Oligo (e.g. CpG) Concentration and Relative Encapsulation Efficiency Determination by Fluorescent Binding Dye.

Materials: Quant-iT OliGreen reagent (Thermo #07582), 96-well plates with lids, PBS, dissociation buffer (PBS, pH 7.4 with 10% DMSO and 1% (wt/wt) Zwittergent 3-14 (Sigma-Aldrich #693017), CpG oligo, general pipette tips & repeater pipette tips. Based on the UV absorption of this stock solution at 260 nm, the oligo concentration was calculated using the absorbance characteristics of this DNA oligo provided by the vendor of 1 O.D=33 μg/mL. A dilution to 2 μg/mL was made in PBS, pH 7.4. From the stock solution at 2 μg/mL, diluted standards were prepared and total CpG concentration and % E.E calculated in the same manner as described for mRNA analysis in Example 3.

### LNP Particle Size

30 μL of LNP was mixed with 1.5 mL PBS in a polystyrene cuvette (Sarstedt, #67.754) and analyzed for size using a ZetaSizer Pro (Malvern) using ZS Xplorer software, version number 1.4.0.105. The Z-average size and polydispersity index value were recorded. Typically, size measurements of LNPs were taken post LNP mixing, post buffer exchange and post sterile filtering.

### LNP Zeta Potential

30 μL of LNP was mixed with 1.5 mL PBS and injected into a disposable folded capillary cell (Malvern Nanoseries DTS1070) and zeta potential measured on a ZetaSizer Pro at 25° C.

### Freeze/Thaw Protocol

To simulate conditions of storage and sample preparation steps involved before dosing animals, a Freeze/Thaw Protocol was established. LNP freezing is accomplished by partial immersion of sample vial in liquid nitrogen (LN2) for 5 min, followed by storage at −80° C. Before dosing, a vial of each LNP is placed in a floater and put in a 37° C. water bath for −30 seconds until the ice has almost fully melted inside the vials. LNPs are diluted with an equal volume of 15 mM Tris, pH 7.4 prior to dosing. UUGU oligo encapsulation efficiency and LNP size were compared after buffer exchange by centrifugal concentrators and 0.2 um filtering (Post-filter) and freeze/thaw cycle (Post F/T).

TABLE 40
Physicochemical properties and characterization of LNP formulations loaded with UUGU oligo.

| | | | | | Post F/T | | | | | |
|---|---|---|---|---|---|---|---|---|---|---|
| LNP | Post-filter | | | | | | | | Zeta Potential | Zeta Potential |
| formulation | Z-Ave | PDI | % EE | stdev | Z-Ave | PDI | % EE | stdev | (mV) pH 5 | (mV) pH 7 |
| KC3-OA¹ | 93.4 | 0.01 | 96.7 | 5.8 | 91.61 | 0.03 | 92.8 | 1.8 | 23.15 | 2.3 |
| KC3-OA/PS² | 92.27 | 0.08 | 93.5 | 3.6 | 93.11 | 0.07 | 91.7 | 3.8 | 21.28 | 1.83 |

Abbreviations:
¹Non-targeted KC3-OA/DSPC/Chol/PEG-DMG at 48/10/40.5/1.5 mol %;
²Targeted KC3-OA/DSPC/DPPS/Chol/PEG-DMG 48/5/5/40.5/1.5 mol %.
% EE, UUGU oligo encapsulation efficiency; stdev, % EE standard deviation; post C/F, post buffer exchange, concentration and filtration using 0.2-μm filter; post F/T, after freeze-thaw cycle; Z-Ave, z-average particle size by DLS, nm; PDI, size polydispersity index. Zeta refers to zeta potential measurements at either pH 5 or pH 7 in units of mV.

As shown in Table 40, the LNP formulations used in this study were <100 nm in diameter and displayed good UUGU oligo entrapment above 90%. The formulations were freeze-thawed to simulate conditions of sample preparation that were performed prior to dosing the animals. After freezing and thawing % EE changed less than 3% and particle size less than 1.5%.

### Example 21. In Vivo Inflammatory Profile of a Synthetic Nucleic Acid TLR7/8 Agonist Delivered by a PS-Targeted Lipid Nanoparticle

The primary aim of this study was to determine if the biological activity of a TLR7/8 UUGU oligonucleotide improved when delivered in an LNP versus unencapsulated or “naked”. A secondary aim was to determine if the efficiency of delivery improved with the PS-targeting of LNPs. The study design is shown in Table 2. All LNPs were prepared with the based formulation of KC3-OA/DSPC/DPPS-NH4/Chol/PEG-DMG (48/5/5/40.5/1.5 mol %). In groups 3 and 5, non-targeted LNPs were formulated by replacing DPPS with 5 mol % DSPC for a total of 10 mol % DSPC.

TABLE 41
Table 41. Study design. BALB/c mice, 5 per group, were
injected with UUGU oligonucleotides encapsulated in
LNPs. Groups 6 and 7 received unencapsulated “naked”
UUGU oligos. Injections were given i.m. in a volume of
20 μL into the left caudal thigh. n/a, not applicable.

| Group | LNP composition | Oligo | Oligo dose |
|---|---|---|---|
| 1 | KC3OA-DPPS | UUGU | 0.1 |
| 2 | KC3OA-DPPS | UUGU | 0.3 |
| 3 | KC3OA | UUGU | 0.3 |
| 4 | KC3OA-DPPS | UUGU | 1 |
| 5 | KC3OA | UUGU | 1 |
| 6 | n/a | UUGU | 1 ug UUGU oligo |
| 7 | n/a | UUGU | 10 ug UUGU oligo |
| 8 | n/a (vehicle) | n/a | n/a |

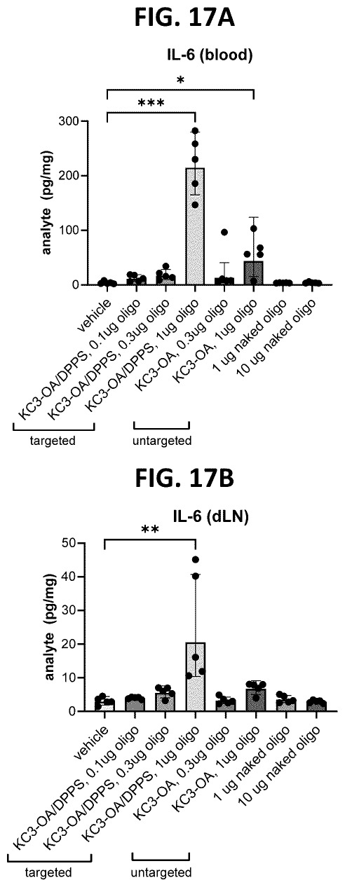

Blood and draining lymph nodes (ipsilateral, inguinal and iliac LNs) were collected 6 hrs post injection. Serum was isolated in serum isolation tubes using standard protocols. LNs were placed in microcentrifuge tubes containing 0.25 mL T-PER tissue lysis buffer (ThermoFisher, Cat No. 78510) containing Halt Protease Inhibitor Cocktail (ThermoFisher, Cat. No. 78441) and Universal Nuclease for Cell Lysis (ThermoFisher, Cat. No. 88700) and lysed using a pestle. Samples were centrifuged at 10,000×g for 10 min to pellet all non-soluble material and clarified supernatants were harvested. Cytokines and chemokines were quantified using the LEGENDplex 13-plex Mouse Anti-Virus Response Panel (BioLegend, Cat. No. 740622). Total protein content in the lysis supernatant was quantified using detergent-compatible Bradford reagent at absorbance of OD595. The pg/mL concentration of analytes from the LEGENDplex were normalized to 1 mg/mL total protein content and reported as pg/mg of protein. Results are shown in FIGS. 17A to 23B as the geometric mean and geometric SD. Statistical changes relative to vehicle were calculated using the nonparametric Krustal-Wallis test with Dunn's multiple comparisons post-test.

Expression levels of analytes are summarized for the blood in Table 42 and for the dLNs in Table 43.

TABLE 42
Concentrations of cytokine analytes in the blood following administration
of UUGU oligoribonucleotides encapsulated in LNPs. Data shown as Mean (SD).

| Group (blood) | IFN-γ | | IL-6 | | TNF-α | | IL-1β | | IL-12p70 | | IL-10 | | IFN-α | |
|---|---|---|---|---|---|---|---|---|---|---|---|---|---|---|
| vehicle | 1.7 | (0.5) | 4 | (2.5) | 10.7 | (8.7) | 1.6 | (1) | 1.3 | (0.7) | 5.3 | (0) | 8.3 | (3.9) |
| KC3-OA/DPPS | | | | | | | | | | | | | | |
| 0.1 ug oligo | 4.8 | (2.6) | 12.4 | (5.9) | 15.8 | (7.2) | 2.7 | (1.9) | 2.2 | (1.1) | 18.2 | (18) | 15.1 | (4.3) |
| 0.3 ug oligo | 7 | (6.4) | 18.8 | (9.8) | 13.1 | (1.2) | 1.1 | (0.5) | 0.6 | (0.2) | 8.6 | (7.4) | 22.3 | (12.8) |
| 1 ug oligo | 20.1 | (10) | 220.7 | (54.9) | 17.5 | (4.1) | 3 | (1.8) | 1.6 | (0.8) | 4.5 | (1.7) | 38.1 | (6.8) |
| KC3-OA | | | | | | | | | | | | | | |
| 0.3 ug oligo | 3.2 | (0.8) | 25.8 | (39.6) | 15 | (4.3) | 1.6 | (1.4) | 1.1 | (0.7) | 8.2 | (6.6) | 8.8 | (2.6) |
| 1 ug oligo | 4.3 | (2.1) | 58.2 | (34.5) | 17.8 | (4.3) | 3.8 | (4.3) | 1.6 | (1.1) | 8.1 | (6.3) | 18.6 | (9.4) |
| Naked Oligo | | | | | | | | | | | | | | |
| 1 ug | 2.8 | (1.5) | 3.7 | (0.4) | 8.4 | (2.2) | 0.9 | (0.3) | 0.9 | (0.5) | 9.5 | (10) | 7.7 | (3.9) |
| 10 ug | 2.3 | (0.8) | 4.1 | (1.2) | 8.7 | (2.7) | 0.6 | (0.3) | 1.4 | (0.5) | 9.6 | (9.8) | 6.9 | (1.9) |

| Group (blood) | IP-10/CXCL10 | | GM-CSF | | KC | | MCP-1/CCL2 | | RANTES/CCL5 | |
|---|---|---|---|---|---|---|---|---|---|---|
| vehicle | 392.3 | (111.7) | 3.3 | (1.5) | 58.3 | (20.5) | 21.8 | (23.6) | 8.7 | (8.8) |
| KC3-OA/DPPS | | | | | | | | | | |
| 0.1 ug oligo | 483.2 | (116.3) | 3.4 | (2.1) | 58.6 | (12.7) | 47.2 | (24.9) | 18.2 | (6.4) |
| 0.3 ug oligo | 670.2 | (158.1) | 2.5 | (2.2) | 131 | (96.4) | 67.1 | (13.1) | 22.4 | (10.7) |
| 1 ug oligo | 1217.4 | (83.5) | 3.1 | (1.2) | 476.1 | (94.3) | 174.6 | (15.2) | 23.1 | (9.5) |
| KC3-OA | | | | | | | | | | |
| 0.3 ug oligo | 575.3 | (100) | 3.2 | (1.3) | 112.7 | (62.4) | 72.5 | (21.5) | 38.4 | (20.9) |
| 1 ug oligo | 999.7 | (272.9) | 3.3 | (1.2) | 175.9 | (69.3) | 112.6 | (33.1) | 37.2 | (34.9) |
| Naked Oligo | | | | | | | | | | |
| 1 ug | 413.9 | (89.8) | 4 | (0) | 92.5 | (100) | 31.2 | (15.4) | 12.8 | (10.8) |
| 10 ug | 343 | (58.1) | 3.3 | (1.4) | 53.8 | (7.2) | 22.7 | (3.5) | 11.1 | (6.7) |

TABLE 43
Concentrations of cytokine analytes (pg per mg of protein) in the draining LNs following administration
of UUGU oligoribonucleotides encapsulated in LNPs. Data shown as Mean (SD).

| Group (dLN) | IFN-γ | | IL-6 | | TNF-α | | IL-1β | | IL-12p70 | | IL-10 | | IFN-α | |
|---|---|---|---|---|---|---|---|---|---|---|---|---|---|---|
| vehicle | 5.4 | (1.1) | 3.1 | (1.1) | 6.1 | (3.7) | 13.6 | (7.4) | 2 | (1) | 4.1 | (1.7) | 26.9 | (26.7) |
| KC3-OA/DPPS | | | | | | | | | | | | | | |
| 0.1 ug oligo | 19.4 | (11.4) | 4 | (0.3) | 20.2 | (5) | 31.5 | (8.1) | 3.7 | (2.5) | 12.3 | (5.8) | 24.4 | (9.1) |
| 0.3 ug oligo | 65.4 | (54.1) | 5.7 | (1.6) | 15.6 | (3) | 33 | (7.5) | 2.5 | (0.9) | 8.1 | (4) | 30.3 | (21.2) |
| 1 ug oligo | 231.6 | (144.5) | 24.8 | (16.5) | 26.1 | (3.3) | 91.1 | (23.2) | 2 | (0.6) | 6 | (2.5) | 55.6 | (16.2) |
| KC3-OA | | | | | | | | | | | | | | |
| 0.3 ug oligo | 10.8 | (3.8) | 3.3 | (1) | 15.4 | (3.8) | 22.6 | (5) | 2.9 | (1.3) | 9.2 | (4.9) | 23.7 | (9) |
| 1 ug oligo | 39.6 | (40.8) | 7 | (1.7) | 14.6 | (4.7) | 44.9 | (12) | 2.9 | (1.3) | 7.5 | (5.2) | 35.6 | (14.4) |
| Naked Oligo | | | | | | | | | | | | | | |
| 1 ug | 7 | (4.3) | 3.6 | (1.1) | 5.8 | (3.9) | 12.5 | (2.8) | 2.8 | (1.9) | 6 | (1.7) | 13.7 | (2.3) |
| 10 ug | 6.1 | (1) | 2.9 | (0.4) | 9.3 | (1.4) | 11.5 | (2.6) | 2 | (1) | 5 | (0.7) | 17 | (5.5) |

| Group (dLN) | IP-10/CXCL10 | | GM-CSF | | KC | | MCP-1/CCL2 | | RANTES/CCL5 | |
|---|---|---|---|---|---|---|---|---|---|---|
| vehicle | 110.1 | (25.7) | 2.9 | (1.3) | 1.7 | (1) | 13.5 | (8.5) | 590.2 | (94.1) |
| KC3-OA/DPPS | | | | | | | | | | |
| 0.1 ug oligo | 1217.4 | (613.2) | 1.8 | (1) | 3.6 | (0.9) | 60.3 | (19.1) | 677.8 | (119.4) |
| 0.3 ug oligo | 1424.8 | (520.5) | 3.2 | (0.9) | 7.8 | (6.6) | 70.4 | (27.2) | 585.8 | (75) |
| 1 ug oligo | 4990.7 | (2102.6) | 5 | (1.5) | 23.9 | (6.4) | 148.9 | (42.6) | 548.1 | (33.3) |
| KC3-OA | | | | | | | | | | |
| 0.3 ug oligo | 1114.5 | (610.8) | 2.3 | (1.3) | 6 | (3.2) | 59.9 | (24.8) | 578.6 | (65.5) |
| 1 ug oligo | 2554.7 | (1336.9) | 4 | (3.3) | 7.6 | (3) | 93.8 | (45.2) | 547.7 | (73.7) |
| Naked Oligo | | | | | | | | | | |
| 1 ug | 132.9 | (40.3) | 2.4 | (2.1) | 1.3 | (0.4) | 17.4 | (7.6) | 639.9 | (99.4) |
| 10 ug | 118.5 | (43.2) | 1.9 | (1.5) | 2.3 | (0.5) | 11.7 | (5.1) | 595.2 | (92.5) |

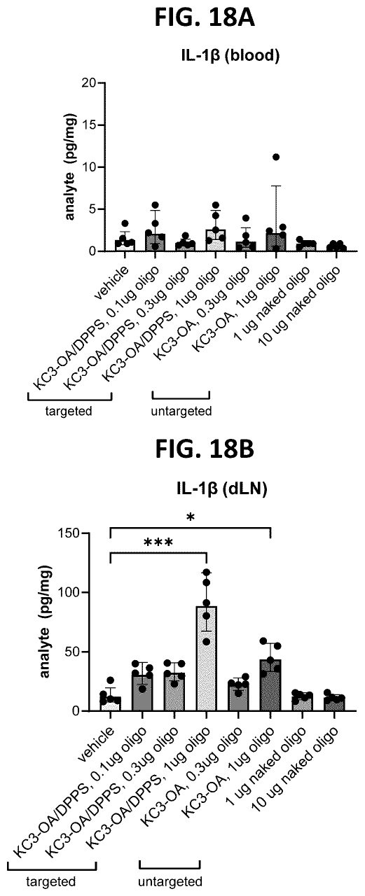

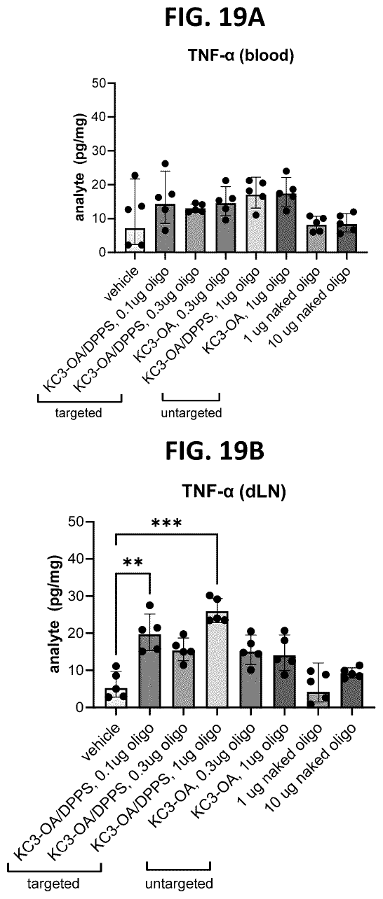

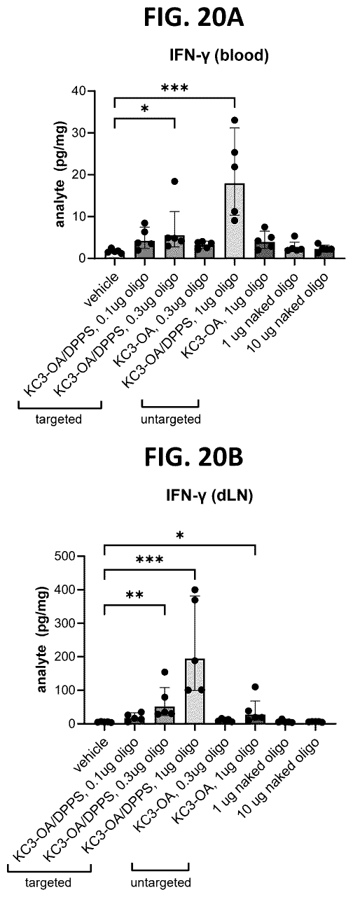

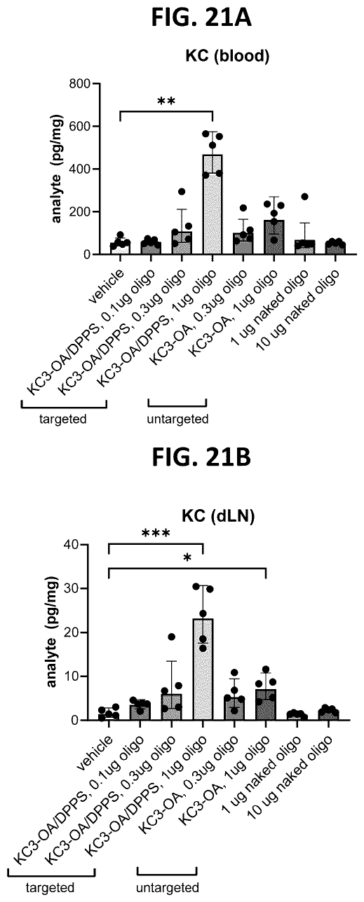

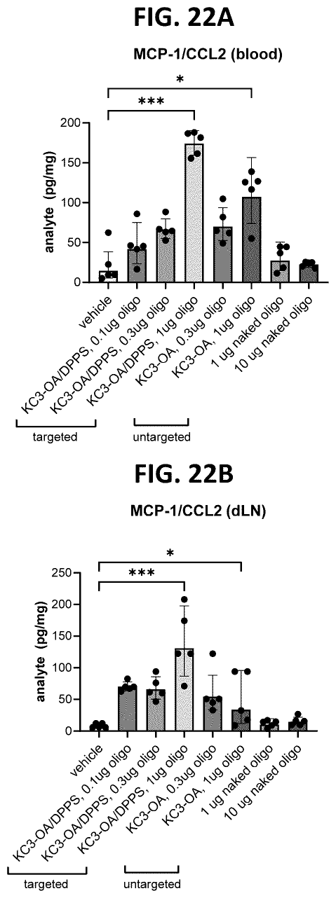

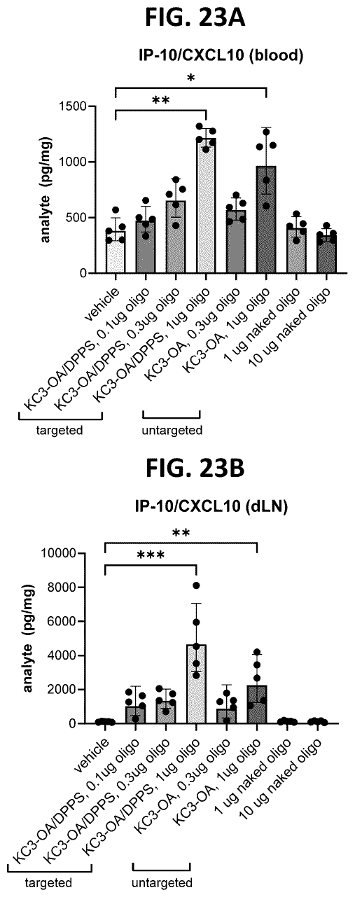

Injection with 1 or 10 g unencapsulated “naked” UUGU oligos did not increase any analyte above vehicle, indicating that the oligos did not efficiently enter into the endolysosomal compartments of innate immune cells expressing TLR7. This is likely due to a combination of rapid degradation by nucleases, poor accumulation in the dLNs, and inefficient uptake by TLR7 expressing innate immune cells (Nguyen D N et al. Proc Natl Acad Sci USA. 2012 Apr. 3; 109(14):E797-803; PMID: 22421433). In contrast, 1 g UUGU delivered by DPPS-targeted LNPs significantly increased the cytokines IL-6 in the blood (FIG. 17A) and dLNs (FIG. 17B), IL-1β in the blood (FIG. 18A) and dLNs (FIG. 18B), TNF-α in the blood (FIG. 19A) and dLNs (FIG. 19B), IFN-γ in the blood (FIG. 20A) and dLNs (FIG. 32B), the neutrophil chemoattractant KC in the blood (FIG. 21A) and dLNs (FIG. 21B), the monocyte chemoattractant CCL2 in the blood (FIG. 22A) and dLNs (FIG. 22B), and CXCL10 in the blood (FIG. 23A) and dLNs (FIG. 23B). The lower doses of 0.1 and 0.3 μg UUGU encapsulated in DPPS-targeted LNPs increased expression of some analytes (e.g. TNF-α in FIGS. 19A-19B and IFN-γ in FIGS. 20A-20B), but at lower levels compared to the 1 g dose. The fold differences in mean analyte concentrations relative to vehicle control are shown for the blood in Table 44A and dLNs in Table 44B. Statistical comparisons between the targeted KC3-OA/DPPS and non-targeted KC3-OA LNPs are shown for each analyte in the blood and dLNs in Table 45. In conclusion, LNP delivery of UUGU oligoribonucleotides to TLR7/8-expressing innate allowed for significantly reduced doses relative to unencapsulated UUGU to induce biological activity. Additionally, PS-targeting of UUGU-containing LNPs was important to the increase in delivery efficiency: the 1 g dose of UUGU encapsulated in non-targeted LNPs was consistently lower relative to PS-targeted LNPs.

TABLE 44A
Fold differences in mean analyte concentrations
relative to vehicle control in blood.

| Group (blood) | IFN-γ | KC | TNF-α | MCP-1 | RANTES |
|---|---|---|---|---|---|
| vehicle | 1 | 1 | 1 | 1 | 1 |
| KC3-OA/DPPS | | | | | |
| 0.1 ug oligo | 2.8 | 1 | 1.5 | 2.2 | 2.1 |
| 0.3 ug oligo | 4.1 | 2.2 | 1.2 | 3.1 | 2.6 |
| 1 ug oligo | 11.8 | 8.2 | 1.6 | 8 | 2.7 |
| KC3-OA | | | | | |
| 0.3 ug oligo | 1.9 | 1.9 | 1.4 | 3.3 | 4.4 |
| 1 ug oligo | 2.5 | 3 | 1.7 | 5.2 | 4.3 |
| Naked Oligo | | | | | |
| 1 ug | 1.6 | 1.6 | 0.8 | 1.4 | 1.5 |
| 10 ug | 1.4 | 0.9 | 0.8 | 1 | 1.3 |

| Group (blood) | IL-1β | IP-10 | GM-CSF | IFN-α | IL-6 |
|---|---|---|---|---|---|
| vehicle | 1 | 1 | 1 | 1 | 1 |
| KC3-OA/DPPS | | | | | |
| 0.1 ug oligo | 1.7 | 1.2 | 1 | 1.8 | 3.1 |
| 0.3 ug oligo | 0.7 | 1.7 | 0.8 | 2.7 | 4.7 |
| 1 ug oligo | 1.9 | 3.1 | 0.9 | 4.6 | 55.2 |
| KC3-OA | | | | | |
| 0.3 ug oligo | 1 | 1.5 | 1 | 1.1 | 6.5 |
| 1 ug oligo | 2.4 | 2.5 | 1 | 2.2 | 14.6 |
| Naked Oligo | | | | | |
| 1 ug | 0.6 | 1.1 | 1.2 | 0.9 | 0.9 |
| 10 ug | 0.4 | 0.9 | 1 | 0.8 | 1 |

TABLE 44B
Fold differences in mean analyte concentrations
relative to vehicle control in lymph nodes.

| Group (blood) | IFN-γ | KC | TNF-α | MCP-1 | RANTES |
|---|---|---|---|---|---|
| vehicle | 1 | 1 | 1 | 1 | 1 |
| KC3-OA/DPPS | | | | | |
| 0.1 ug oligo | 3.6 | 2.1 | 3.3 | 4.5 | 1.1 |
| 0.3 ug oligo | 12.1 | 4.6 | 2.6 | 5.2 | 1.0 |
| 1 ug oligo | 42.9 | 14.1 | 4.3 | 11.0 | 0.9 |
| KC3-OA | | | | | |
| 0.3 ug oligo | 2.0 | 3.5 | 2.5 | 4.4 | 1.0 |
| 1 ug oligo | 7.3 | 4.5 | 2.4 | 6.9 | 0.9 |
| Naked Oligo | | | | | |
| 1 ug | 1.3 | 0.8 | 1.0 | 1.3 | 1.1 |
| 10 ug | 1.1 | 1.4 | 1.5 | 0.9 | 1.0 |

| Group (blood) | IL-1β | IP-10 | GM-CSF | IFN-α | IL-6 |
|---|---|---|---|---|---|
| vehicle | 1 | 1 | 1 | 1 | 1 |
| KC3-OA/DPPS | | | | | |
| 0.1 ug oligo | 2.3 | 11.1 | 0.6 | 0.9 | 1.3 |
| 0.3 ug oligo | 2.4 | 12.9 | 1.1 | 1.1 | 1.8 |
| 1 ug oligo | 6.7 | 45.3 | 1.7 | 2.1 | 8.0 |
| KC3-OA | | | | | |
| 0.3 ug oligo | 1.7 | 10.1 | 0.8 | 0.9 | 1.1 |
| 1 ug oligo | 3.3 | 23.2 | 1.4 | 1.3 | 2.3 |
| Naked Oligo | | | | | |
| 1 ug | 0.9 | 1.2 | 0.8 | 0.5 | 1.2 |
| 10 ug | 0.8 | 1.1 | 0.7 | 0.6 | 0.9 |

TABLE 45
Statistical comparisons between the targeted KC3-OA/DPPS and non-
targeted KC3-OA LNPs are shown for each analyte in the blood and
dLNs. Statistical methods: nonparametric Mann-Whitney test with
FDR multiple comparisons test using the Two-stage setup (Benjamini,
Krieger, and Yekutieli). Q value set at 5%. ns, not significant.

| Group (blood) | IFN-γ | KC | TNF-α | MCP-1 | RANTES |
|---|---|---|---|---|---|
| blood | 0.010 | 0.010 | ns | 0.010 | ns |
| dLN | 0.040 | 0.017 | 0.017 | ns | ns |

| Group (blood) | IL-1β | IP-10 | GM-CSF | IFN-α | IL-6 |
|---|---|---|---|---|---|
| blood | ns | ns | ns | 0.017 | 0.010 |
| dLN | 0.025 | ns | ns | ns | 0.017 |

### Example 22. In Vivo Inflammatory Profile of a Synthetic Nucleic Acid TLR9 Agonist Delivered by a PS-Targeted Lipid Nanoparticle

The primary aim of this study was to identify an in vivo dose range of the TLR9 agonists CpG 1018 and CpG 2006 when delivered by a PS-targeted LNP. The secondary aim was to determine whether the biological activity of CpG was increased when delivered by LNP compared to non-encapsulated free CpG-1018; in other words, is CpG more potent at lower doses when delivered by LNPs. The proinflammatory profiles of CpG-LNPs were examined in an mRNA vaccine setting where all treatment groups received LNPs encapsulated with mRNA encoding for the SARS-CoV-2 Spike protein (SEQ ID NO: 1). The study design is shown in Table 47. All LNPs were prepared with the based formulation of KC3-OA/DSPC/DPPS-NH4/Chol/PEG-DMG (48/5/5/40.5/1.5 mol %). Nucleic acids were incorporated into LNPs at an N/P ratio of 5.25.

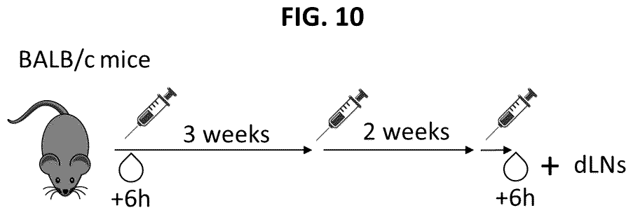

FIG. 10 is a schematic for an in vivo murine inflammatory profile of a synthetic nucleic acid TLR9 agonist delivered by a PS targeted lipid nanoparticle (Example 22).

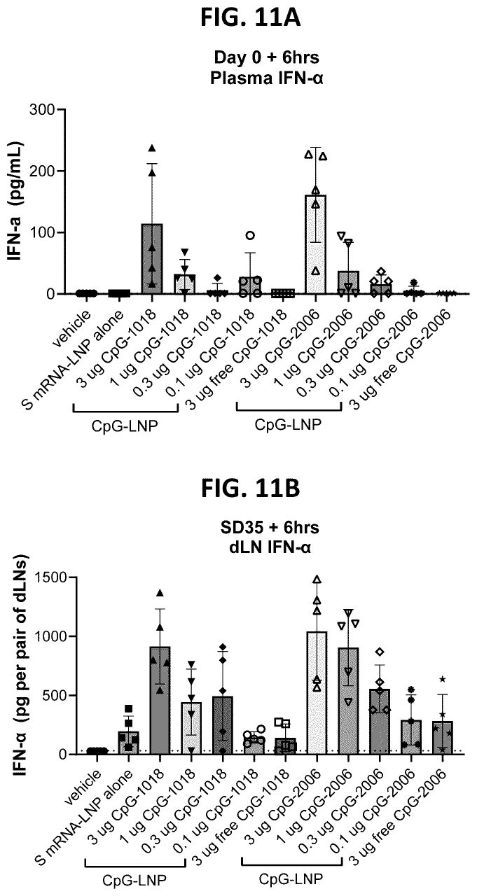

FIG. 11A is a graph showing IFN-α detected in plasma of mice in an in vivo murine inflammatory profile of a synthetic nucleic acid TLR9 agonist delivered by a PS targeted lipid nanoparticle (Example 22). FIG. 11B is a graph showing INF-α detected in draining lymph nodes of mice in an in vivo murine inflammatory profile of a synthetic nucleic acid TLR9 agonist delivered by a PS targeted lipid nanoparticle (Example 22).

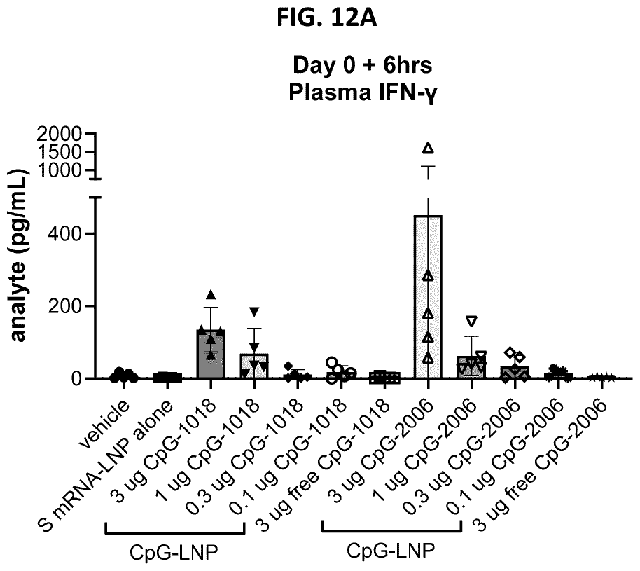

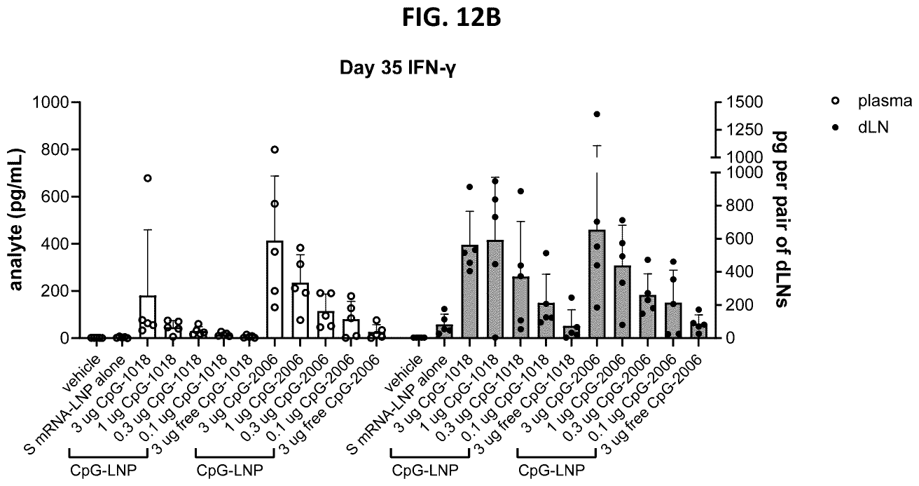

FIG. 12A is a graph showing IFN-γ detected in plasma of mice in an in vivo murine inflammatory profile of a synthetic nucleic acid TLR9 agonist delivered by a PS targeted lipid nanoparticle (Example 22). FIG. 12B is a graph showing IFN-γ detected in draining lymph nodes of mice in an in vivo murine inflammatory profile of a synthetic nucleic acid TLR9 agonist delivered by a PS targeted lipid nanoparticle (Example 22).

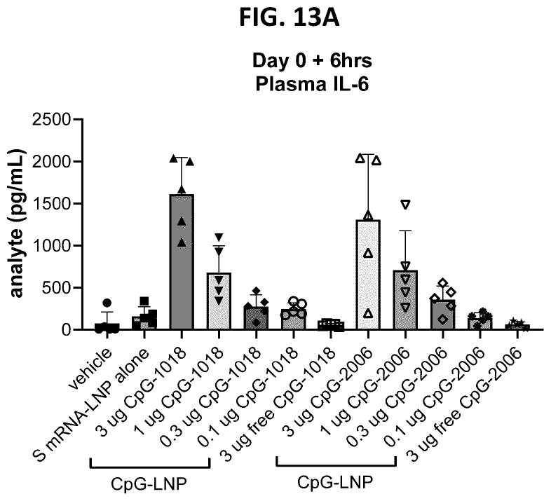

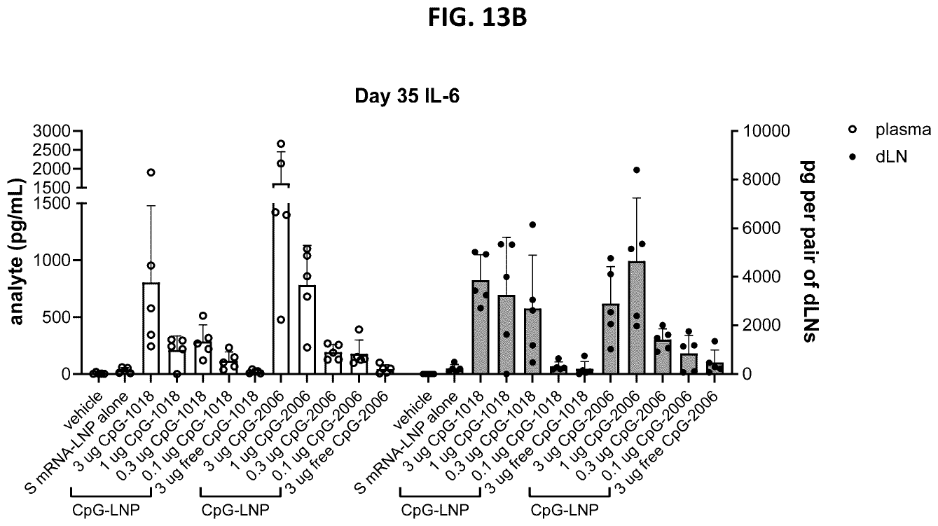

FIG. 13A is a graph showing IL-6 detected in plasma of mice in an in vivo murine inflammatory profile of a synthetic nucleic acid TLR9 agonist delivered by a PS targeted lipid nanoparticle (Example 22). FIG. 13B is a graph showing IL-6 detected in draining lymph nodes of mice in an in vivo murine inflammatory profile of a synthetic nucleic acid TLR9 agonist delivered by a PS targeted lipid nanoparticle (Example 22).

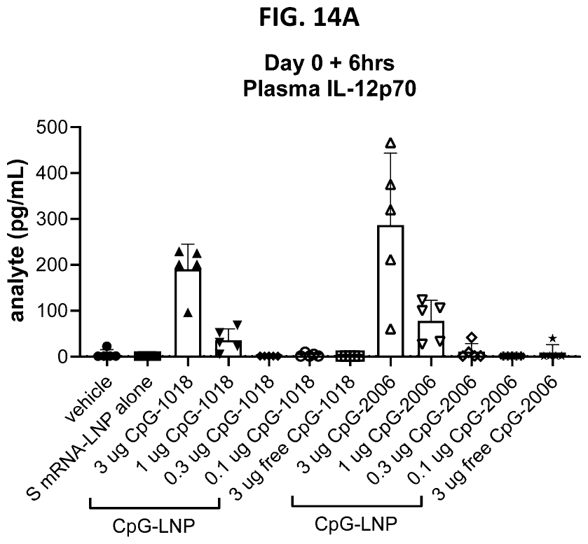

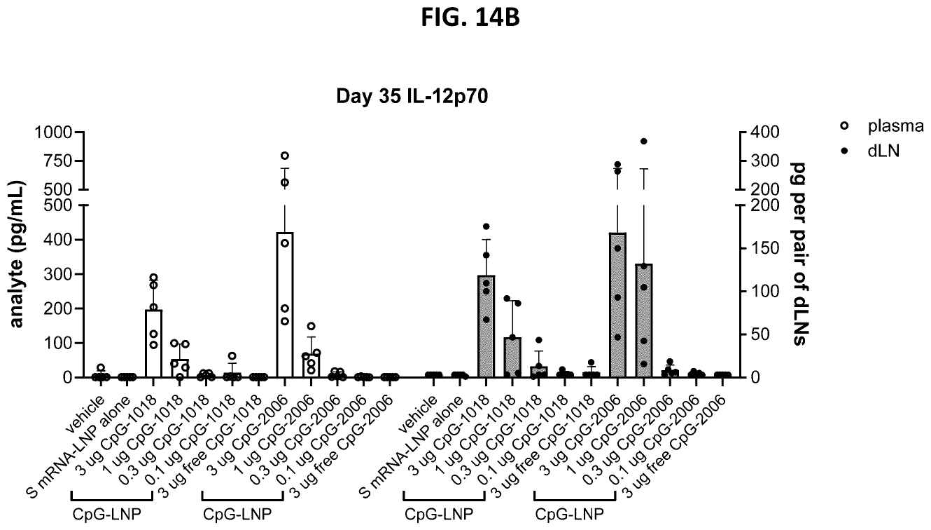

FIG. 14A is a graph showing IL-12p70 detected in plasma of mice in an in vivo murine inflammatory profile of a synthetic nucleic acid TLR9 agonist delivered by a PS targeted lipid nanoparticle (Example 22). FIG. 14B is a graph showing IL-12p70 detected in draining lymph nodes of mice in an in vivo murine inflammatory profile of a synthetic nucleic acid TLR9 agonist delivered by a PS targeted lipid nanoparticle (Example 22).

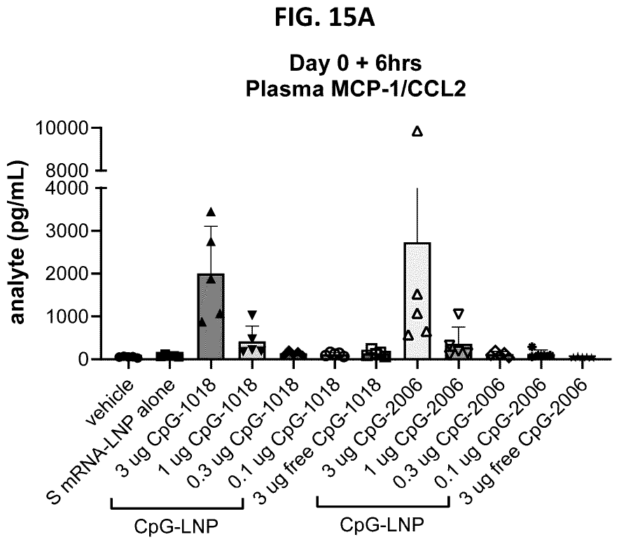

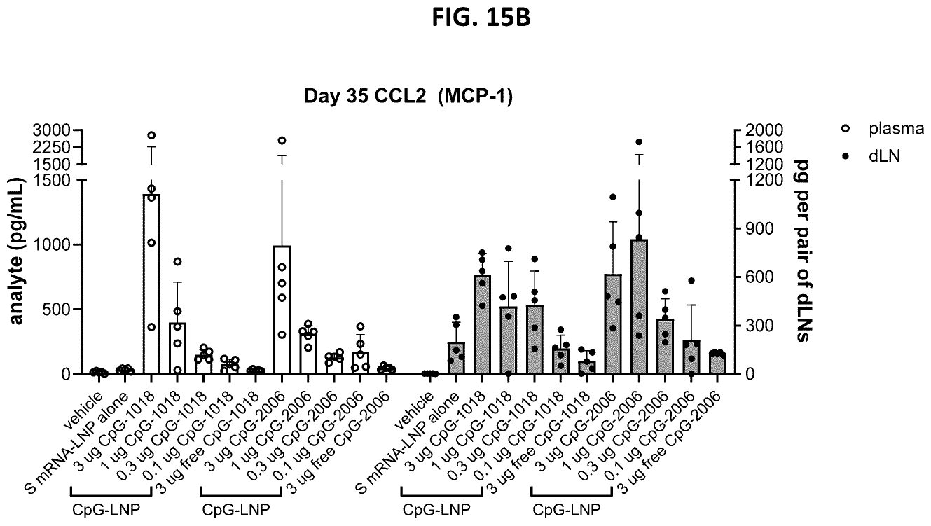

FIG. 15A is a graph showing chemokine CCL2/MCP-1 detected in plasma of mice in an in vivo murine inflammatory profile of a synthetic nucleic acid TLR9 agonist delivered by a PS targeted lipid nanoparticle (Example 22). FIG. 15B is a graph showing chemokine CCL2/MCP-1 detected in draining lymph nodes of mice in an in vivo murine inflammatory profile of a synthetic nucleic acid TLR9 agonist delivered by a PS targeted lipid nanoparticle (Example 22).

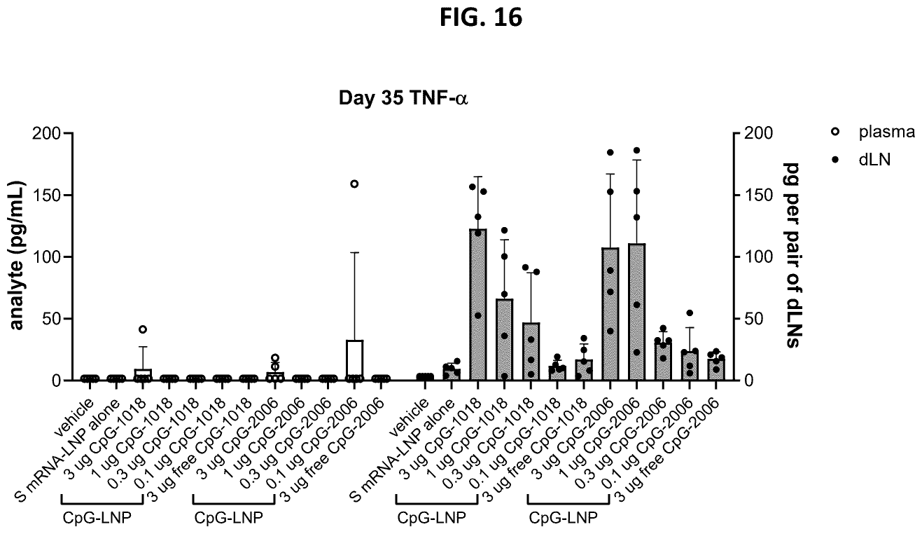

FIG. 16 is a graph showing TNF-α detected in plasma and draining lymph nodes of mice in an in vivo murine inflammatory profile of a synthetic nucleic acid TLR9 agonist delivered by a PS targeted lipid nanoparticle (Example 22). FIG. 17A is a graph showing levels of cytokine IL-6 in the blood of mice observed in the in vivo inflammatory profile of a synthetic nucleic acid TLR 7/8 agonist delivered by a PS-targeted nanoparticle (Example 21).

TABLE 46
Physicochemical properties and characterization of LNPs loaded with CpG-ODN oligos.

| | | Post-Filter | | Post F/T | | Post-Filter | | zeta | |
|---|---|---|---|---|---|---|---|---|---|
| Formulation | nucleic acid | Z-Average | PDI | Z-Average | PDI | % EE | stdev | ph 5 | ph 7 |
| KC3OA/5% PS | mRNA | 110.70 | 0.08 | 111.50 | 0.08 | 91.1 | 6.1 | 10.02 | 0.16 |
| | (SEQ ID NO: 1) | | | | | | | | |
| KC3OA/5% PS | CpG-1018 oligo | 97.03 | 0.07 | 96.06 | 0.10 | 89.8 | 4.6 | 15.46 | 0.19 |
| | (SEQ ID NO: 2) | | | | | | | | |
| KC3OA/5% PS | CpG-2006 oligo | 105.50 | 0.10 | 107.40 | 0.15 | 83.7 | 5.8 | 14.73 | 0.08 |
| | (SEQ ID NO: 3) | | | | | | | | |

% EE, oligonucleotide encapsulation efficiency; stdev, % EE standard deviation; post-mix, after mixing of the lipids and UUGU oligo solutions; post filter, after buffer exchange by centrifugal concentrators and 0.2-μm filtration; post F/T, after freeze-thaw cycle; Z-Ave, z-average particle size by DLS, nm; PDI, size polydispersity index. Zeta refers to zeta potential measurements at either pH 5 or pH 7 in units of mV.

TABLE 47
Study design to examine in vivo inflammatory profiles of CpG-
ODNs. All BALB/c mice except for Group 1 received Spike mRNA-
LNPs. Group 3-12 were co-administered CpG-1018 or CpG-2006
encapsulated in LNPs with the exception of Groups 7 and 12,
which received non-encapsulated “naked” CpG. Injections
were i.m. in the left caudal thigh in a volume of 50 μL.

| | | | Adjuvants co-administered | |
|---|---|---|---|---|
| | vaccine | | with vaccine | |
| | | S mRNA | CpG | CpG |
| Group | immunization | dose | type | dose (ug) |
| 1 | vehicle | n/a | n/a | n/a |
| 2 | Spike | 0.3 | vehicle | |
| 3 | mRNA-LNPs | | 1018 | 3 |
| 4 | | | 1018 | 1 |
| 5 | | | 1018 | 0.3 |
| 6 | | | 1018 | 0.1 |
| 7 | | | 1018 | 3 (naked) |
| 8 | | | 2006 | 3 |
| 9 | | | 2006 | 1 |
| 10 | | | 2006 | 0.3 |
| 11 | | | 2006 | 0.1 |
| 12 | | | 2006 | 3 (naked) |

All groups were given a prime/boost/boost vaccination regimen with the 1^{st} boost occurring on day 21 and the 2^{nd} boost occurring on day 35. Blood was collected 6 hrs after the initial injection (Day 0+6 hrs). After the 2^{nd} boost on day 35, blood and draining lymph nodes (dLNs; ipsilateral inguinal and iliac LNs) were collected 6 hrs post injection (Day 35+6 hrs) (FIG. 10). Plasma was isolated in lithium heparin tubes. LNs were lysed in T-PER buffer as described in Example 3, the only difference being that 0.2 mL T-PER buffer was used. Type I IFN-α was quantified using the VeriKine Mouse IFN-alpha ELISA kit (PBL Assay Science, Cat. No. 42120-1). A 10-plex Luminex Discovery Assay (Eve Technologies, Calgary, Cat. No. MDF10) was used to determine analyte levels of canonical acute pro-inflammatory factors.

Non-encapsulated CpG ODN is typically co-administered to mice as an adjuvant in a dose range >25 μg (Pham N L et al. J Immunol. 2009 Aug. 15; 183(4):2337-48. PMID: 19635915; Badovinac V P et al. Nat Immunol. 2004 August; 5(8):809-17. PMID: 15247915). High ratios of CpG ODN adjuvant to antigen are required due to the inefficient accumulation in draining LNs and uptake into the endolysosomal compartment of TLR9-expressing innate immune cells (limited to plasmacytoid DCs and B cells in humans; more broadly expressed by monocytes, macrophages and myeloid DC populations in rodents) and short half-life (Liu H et al. Nature. 2014 Mar. 27; 507(7493):519-22. PMID: 24531764; Hartmann G. Adv Immunol. 2017; 133:121-169. PMID: 28215278; Immgen.org; Campbell J D. Methods Mol Biol. 2017; 1494:15-27. PMID: 27718183; Bode C et al. Expert Rev Vaccines. 2011 April; 10(4):499-511. PMID: 21506647). For instance, in the approved hepatitis B vaccine Heplisav B, 3 mg CpG 1018 is co-administered with 20 g HBsAg (Campbell J D et al.).

Injection of Spike mRNA-LNPs alone did not induce detectable IFN-α in the blood on Day 0+6 hrs nor did non-encapsulated “free” CpG-1018 or CpG-2006. In contrast, CpG-LNPs dosed at 3, 1, 0.3 or 0.1 g induced IFN-α in a dose-specific manner (FIG. 11A). Qualitatively similar results were seen on Day 35+6 hrs in the dLNs (FIG. 11B). Spike mRNA-LNPs induced low levels of IFN-α, and 3 μg free CpG or 0.1 μg CpG-LNP did not statistically increase levels further. However, CpG-LNPs at 0.3, 1 and 3 μg showed biological activity. Similar findings were observed for the induction of IFN-γ (FIGS. 12A and 12B), IL-6 (FIGS. 13A and 13B), IL-12p70 (FIGS. 14A and 14B), the chemokine CCL2/MCP-1 (FIGS. 15A and 15B), and TNF-α (FIG. 16): while a 3 g dose of non-encapsulated CpG-1018 or CpG-2006 was insufficient to induce cytokines or chemokines, a dose as low as 0.3 ug CpG-LNP showed biological activity with the induction of most analytes. Table 48 shows analyte pg/mL concentrations in the blood plasma on Day 0+6 hrs. Table 51 shows fold-changes over vehicle controls for plasma analyte concentrations on Day 0+6 hrs. Table 49 shows analyte pg/mL concentrations in the blood plasma on Day 35+6 hrs. Table 52 shows fold-changes over vehicle controls for plasma analyte concentrations on Day 35+6 hrs. Table 50 shows analyte pg/mL concentrations in the dLNs on Day 35+6 hrs. Table 53 shows fold-changes over vehicle controls for dLN tissue analyte concentrations on Day 35+6 hrs. In conclusion, CpG-ODN formulated in PS-targeted LNPs increases the efficiency of CpG to TLR9-expressing innate immune cells. Significantly larger doses of unencapsulated CpG-ODN are necessary to comparable biological activity. We expect that co-administration of PS-targeted CpG-LNPs with vaccine antigens will enhance antigen-specific B cell and T cell immune responses over what non-encapsulated CpG is able to achieve and have a superior therapeutic index and safety profile.

TABLE 48
Summary data of plasma analyte concentrations from Day 0 + 6 hrs post i.m. injection. Data shown as
Mean (SD) of 5 mice. Analyte concentrations that fell below the level of detection were given a value of 1.5.

| Group (Day 0 + 6 hrs) | IFN-a | | IFN-g | | IL-6 | | IL-12p70 | | CCL2 | |
|---|---|---|---|---|---|---|---|---|---|---|
| vehicle | 1.04 | (0) | 7.5 | (7.9) | 74.5 | (137.2) | 5.6 | (9.5) | 52.8 | (14.8) |
| S mRNA-LNP alone | 1.04 | (0) | 2.6 | (1.8) | 159.3 | (115.1) | 1.5 | (0) | 79.4 | (22.6) |
| 3 ug CpG-1018 | 114.2 | (97.8) | 134.6 | (61.3) | 1609.2 | (437.3) | 190.7 | (54.3) | 2006.9 | (1097.3) |
| 1 ug CpG-1018 | 31.9 | (24.4) | 69.2 | (69.1) | 680.2 | (318.8) | 35.9 | (24.9) | 416.2 | (362) |
| 0.3 ug CpG-1018 | 6.1 | (11.3) | 11.2 | (14.1) | 275.1 | (139.6) | 1.4 | (0.2) | 143.7 | (53.9) |
| 0.1 ug CpG-1018 | 28.2 | (38.9) | 18 | (17.4) | 245.3 | (73.7) | 3.9 | (3.6) | 116.4 | (40.7) |
| 3 ug free CpG-1018 | 1.04 | (0) | 2.8 | (2.5) | 57.1 | (16.5) | 1.5 | (0) | 140.1 | (69.2) |
| 3 ug CpG-2006 | 161.3 | (77.2) | 451.4 | (657.4) | 1307.2 | (779.4) | 286.8 | (156.6) | 2736.5 | (4001.5) |
| 1 ug CpG-2006 | 37.8 | (46.4) | 62.8 | (54.1) | 706.6 | (471.5) | 78 | (44.7) | 360.8 | (395.7) |
| 0.3 ug CpG-2006 | 16.1 | (15.1) | 33.2 | (31.2) | 356.4 | (164.3) | 11.2 | (17.4) | 120.2 | (61.8) |
| 0.1 ug CpG-2006 | 4.9 | (7.9) | 14.7 | (11.5) | 138.9 | (64.3) | 1.5 | (0) | 123.9 | (94) |
| 3 ug free CpG-2006 | 1.04 | (0) | 3.1 | (1.8) | 62.4 | (34.6) | 9 | (17.1) | 40.4 | (11.7) |

TABLE 49
Summary data of plasma analyte concentrations from Day 35 + 6 hrs post i.m. injection.
Data shown as Mean (SD) of 5 mice. IFN-α was not assayed in the plasma at this time
point. Analyte concentrations that fell below the level of detection were given a value of 1.5.

| Plasma (D 35 + 6 hrs) | IFN-g | | IL-6 | | IL-12p70 | | CCL2 | | TNF-a | |
|---|---|---|---|---|---|---|---|---|---|---|
| vehicle | 1.4 | (0.3) | 6 | (7.4) | 7 | (12.4) | 14.2 | (7.3) | 1.5 | (0) |
| S mRNA-LNP alone | 3.5 | (2.9) | 32.8 | (23.5) | 1.5 | (0) | 33.7 | (10.7) | 1.5 | (0) |
| 3 ug CpG-1018 | 181.5 | (278.4) | 805.7 | (673.4) | 197 | (85.6) | 1390.7 | (883.3) | 9.5 | (17.8) |
| 1 ug CpG-1018 | 47.4 | (27.7) | 211.7 | (123.3) | 53.6 | (43.4) | 397.5 | (313) | 1.5 | (0) |
| 0.3 ug CpG-1018 | 30.7 | (17.9) | 287.9 | (145.3) | 6.7 | (5.3) | 149.5 | (39.4) | 1.5 | (0) |
| 0.1 ug CpG-1018 | 16 | (7.7) | 117.7 | (76.3) | 14.2 | (27.3) | 75.8 | (38.9) | 1.5 | (0) |
| 3 ug free CpG-1018 | 6.7 | (5.5) | 20.4 | (14.4) | 1.5 | (0) | 28 | (6.8) | 1.5 | (0) |
| 3 ug CpG-2006 | 413.5 | (274.2) | 1621 | (829.3) | 422.5 | (262.8) | 994.4 | (891.3) | 6.8 | (7.7) |
| 1 ug CpG-2006 | 235.8 | (117.9) | 783 | (347.8) | 69.1 | (49) | 308.5 | (67.2) | 1.5 | (0) |
| 0.3 ug CpG-2006 | 115 | (71.8) | 193.8 | (68) | 8.3 | (8.1) | 128.9 | (30.3) | 1.5 | (0) |
| 0.1 ug CpG-2006 | 80.3 | (75.8) | 179.9 | (120) | 1.7 | (1.1) | 172.1 | (133.6) | 33 | (70.5) |
| 3 ug free CpG-2006 | 26.2 | (30.6) | 43.6 | (37.5) | 1.5 | (0) | 46.3 | (12.9) | 1.5 | (0) |

TABLE 50
Summary data of dLN analyte concentrations from Day 35 + 6 hrs post i.m. injection. Data shown as Mean (SD)
of 5 mice. Individual analyte concentrations that fell below the level of detection were given a value of 1.5.

| dLNs (D35 + 6 hrs) | IFN-g | | IL-6 | | IL-12p70 | | CCL2 | | TNF-a | | IFN-a | |
|---|---|---|---|---|---|---|---|---|---|---|---|---|
| vehicle | 3.4 | (0) | 2.6 | (1.7) | 3.4 | (0) | 3.1 | (0.8) | 3.4 | (0) | 31 | (0) |
| S mRNA-LNP | 83.7 | (61.4) | 233.1 | (159.6) | 2.9 | (1) | 198.3 | (122.1) | 9.4 | (4.6) | 195.4 | (130.6) |
| alone | | | | | | | | | | | | |
| 3 ug CpG-1018 | 564.3 | (202) | 3864.9 | (1044) | 118.9 | (41.4) | 615.7 | (129.9) | 122.7 | (42.1) | 914.8 | (317.4) |
| 1 ug CpG-1018 | 593.5 | (379) | 3257 | (2366.5) | 46.7 | (42.4) | 418 | (278.9) | 66.2 | (47.6) | 443.6 | (279.6) |
| 0.3 ug CpG-1018 | 372.3 | (332.1) | 2695.5 | (2191.8) | 13 | (17.8) | 423.7 | (213.4) | 46.8 | (40.4) | 494.4 | (378.1) |
| 0.1 ug CpG-1018 | 213.2 | (173) | 314.6 | (187.4) | 4.6 | (2.7) | 157.6 | (82.2) | 11.9 | (4.5) | 144.6 | (48) |
| 3 ug free CpG-1018 | 73.9 | (98.4) | 213.7 | (302.7) | 6.3 | (6.4) | 80.2 | (63.7) | 17.1 | (12.5) | 141.8 | (116.1) |
| 3 ug CpG-2006 | 654.8 | (453.7) | 2898.7 | (1524.6) | 168.3 | (105.2) | 618.6 | (322.6) | 107.6 | (59.5) | 1043 | (414.2) |
| 1 ug CpG-2006 | 439.4 | (242.7) | 4651.5 | (2596.6) | 132.3 | (140.1) | 833.7 | (593.1) | 111 | (67.3) | 905.4 | (323) |
| 0.3 ug CpG-2006 | 260.8 | (128.1) | 1420.4 | (438.2) | 8.6 | (6) | 339.9 | (124.7) | 30.7 | (8.8) | 555 | (203.5) |
| 0.1 ug CpG-2006 | 214.6 | (195.6) | 850.2 | (738.7) | 4.5 | (1.8) | 207.9 | (219.6) | 23.9 | (18.8) | 292.2 | (213.1) |
| 3 ug free CpG-2006 | 88.3 | (53) | 478.1 | (507.8) | 3.4 | (0.1) | 128.2 | (6.9) | 17.5 | (5.7) | 283.4 | (225) |

TABLE 51
Fold changes of mean analyte pg/mL concentrations for plasma
Day 0 + 6 hr samples relative to vehicle controls.

| Plasma (Day 0 + 6 hrs) | IFN-a | IFN-g | IL-6 | IL-12p70 | CCL2 |
|---|---|---|---|---|---|
| vehicle | 1 | 1 | 1 | 1 | 1 |
| S mRNA-LNP alone | 1 | 0.3 | 2.1 | 0.3 | 1.5 |
| 3 ug CpG-1018 | 109.8 | 17.9 | 21.6 | 34.1 | 38 |
| 1 ug CpG-1018 | 30.7 | 9.2 | 9.1 | 6.4 | 7.9 |
| 0.3 ug CpG-1018 | 5.9 | 1.5 | 3.7 | 0.3 | 2.7 |
| 0.1 ug CpG-1018 | 27.1 | 2.4 | 3.3 | 0.7 | 2.2 |
| 3 ug free CpG-1018 | 1 | 0.4 | 0.8 | 0.3 | 2.7 |
| 3 ug CpG-2006 | 155.1 | 60.2 | 17.5 | 51.2 | 51.8 |
| 1 ug CpG-2006 | 36.3 | 8.4 | 9.5 | 13.9 | 6.8 |
| 0.3 ug CpG-2006 | 15.5 | 4.4 | 4.8 | 2 | 2.3 |
| 0.1 ug CpG-2006 | 4.7 | 2 | 1.9 | 0.3 | 2.3 |
| 3 ug free CpG-2006 | 1 | 0.4 | 0.8 | 1.6 | 0.8 |

TABLE 52
Fold changes of mean analyte pg/mL concentrations for plasma
Day 35 + 6 hr samples relative to vehicle controls.

| Plasma (Day 35 + 6 hrs) | IFN-g | IL-6 | IL-12p70 | CCL2 | TNF-a |
|---|---|---|---|---|---|
| vehicle | 1 | 1 | 1 | 1 | 1 |
| S mRNA-LNP alone | 2.5 | 5.5 | 0.2 | 2.4 | 1 |
| 3 ug CpG-1018 | 129.6 | 134.3 | 28.1 | 97.9 | 6.3 |
| 1 ug CpG-1018 | 33.9 | 35.3 | 7.7 | 28 | 1 |
| 0.3 ug CpG-1018 | 21.9 | 48 | 1 | 10.5 | 1 |
| 0.1 ug CpG-1018 | 11.4 | 19.6 | 2 | 5.3 | 1 |
| 3 ug free CpG-1018 | 4.8 | 3.4 | 0.2 | 2 | 1 |
| 3 ug CpG-2006 | 295.4 | 270.2 | 60.4 | 70 | 4.5 |
| 1 ug CpG-2006 | 168.4 | 130.5 | 9.9 | 21.7 | 1 |
| 0.3 ug CpG-2006 | 82.1 | 32.3 | 1.2 | 9.1 | 1 |
| 0.1 ug CpG-2006 | 57.4 | 30 | 0.2 | 12.1 | 22 |
| 3 ug free CpG-2006 | 18.7 | 7.3 | 0.2 | 3.3 | 1 |

TABLE 53
Fold changes of mean analyte concentrations for dLN
Day 35 + 6 hr samples relative to vehicle controls.

| dLNs (Day 35 + 6 hrs) | IFN-g | IL-6 | IL-12p70 | CCL2 | TNF-a | IFN-a |
|---|---|---|---|---|---|---|
| vehicle | 1 | 1 | 1 | 1 | 1 | 1 |
| S mRNA-LNP alone | 24.6 | 89.7 | 0.9 | 64 | 2.8 | 6.3 |
| 3 ug CpG-1018 | 166 | 1486.5 | 35 | 198.6 | 36.1 | 29.5 |
| 1 ug CpG-1018 | 174.6 | 1252.7 | 13.7 | 134.8 | 19.5 | 14.3 |
| 0.3 ug CpG-1018 | 109.5 | 1036.7 | 3.8 | 136.7 | 13.8 | 15.9 |
| 0.1 ug CpG-1018 | 62.7 | 121 | 1.4 | 50.8 | 3.5 | 4.7 |
| 3 ug free CpG-1018 | 21.7 | 82.2 | 1.9 | 25.9 | 5 | 4.6 |
| 3 ug CpG-2006 | 192.6 | 1114.9 | 49.5 | 199.5 | 31.6 | 33.6 |
| 1 ug CpG-2006 | 129.2 | 1789 | 38.9 | 268.9 | 32.6 | 29.2 |
| 0.3 ug CpG-2006 | 76.7 | 546.3 | 2.5 | 109.6 | 9 | 17.9 |
| 0.1 ug CpG-2006 | 63.1 | 327 | 1.3 | 67.1 | 7 | 9.4 |
| 3 ug free CpG-2006 | 26 | 183.9 | 1 | 41.4 | 5.1 | 9.1 |

Nucleic acid sequence of mRNA encoding the
SARS-CoV-2 spike protein.
SEQ ID NO: 1
GGGAAUAAACUAGUAUUCUUCUGGUCCCCACAGACUCAGAGAGAA
CCCGCCACCAUGUUCGUGUUCCUGGUGCUGCUGCCUCUGGUGUCC
AGCCAGUGUGUGAACCUGACCACCAGAACACAGCUGCCUCCAGCC
UACACCAACAGCUUUACCAGAGGCGUGUACUACCCCGACAAGGUG
UUCAGAUCCAGCGUGCUGCACUCUACCCAGGACCUGUUCCUGCCU
UUCUUCAGCAACGUGACCUGGUUCCACGCCAUCCACGUGUCCGGC
ACCAAUGGCACCAAGAGAUUCGACAACCCCGUGCUGCCCUUCAAC
GACGGGGUGUACUUUGCCAGCACCGAGAAGUCCAACAUCAUCAGA
GGCUGGAUCUUCGGCACCACACUGGACAGCAAGACCCAGAGCCUG
CUGAUCGUGAACAACGCCACCAACGUGGUCAUCAAAGUGUGCGAG
UUCCAGUUCUGCAACGACCCCUUCCUGGGCGUCUACUACCACAAG
AACAACAAGAGCUGGAUGGAAAGCGAGUUCCGGGUGUACAGCAGC
GCCAACAACUGCACCUUCGAGUACGUGUCCCAGCCUUUCCUGAUG
GACCUGGAAGGCAAGCAGGGCAACUUCAAGAACCUGCGCGAGUUC
GUGUUUAAGAACAUCGACGGCUACUUCAAGAUCUACAGCAAGCAC
ACCCCUAUCAACCUCGUGCGGGAUCUGCCUCAGGGCUUCUCUGCU
CUGGAACCCCUGGUGGAUCUGCCCAUCGGCAUCAACAUCACCCGG
UUUCAGACACUGCUGGCCCUGCACAGAAGCUACCUGACACCUGGC
GAUAGCAGCAGCGGAUGGACAGCUGGUGCCGCCGCUUACUAUGUG
GGCUACCUGCAGCCUAGAACCUUCCUGCUGAAGUACAACGAGAAC
GGCACCAUCACCGACGCCGUGGAUUGUGCUCUGGAUCCUCUGAGC
GAGACAAAGUGCACCCUGAAGUCCUUCACCGUGGAAAAGGGCAUC
UACCAGACCAGCAACUUCCGGGUGCAGCCCACCGAAUCCAUCGUG
CGGUUCCCCAAUAUCACCAAUCUGUGCCCCUUCGGCGAGGUGUUC
AAUGCCACCAGAUUCGCCUCUGUGUACGCCUGGAACCGGAAGCGG
AUCAGCAAUUGCGUGGCCGACUACUCCGUGCUGUACAACUCCGCC
AGCUUCAGCACCUUCAAGUGCUACGGCGUGUCCCCUACCAAGCUG
AACGACCUGUGCUUCACAAACGUGUACGCCGACAGCUUCGUGAUC
CGGGGAGAUGAAGUGCGGCAGAUUGCCCCUGGACAGACAGGCAAG
AUCGCCGACUACAACUACAAGCUGCCCGACGACUUCACCGGCUGU
GUGAUUGCCUGGAACAGCAACAACCUGGACUCCAAAGUCGGCGGC
AACUACAAUUACCUGUACCGGCUGUUCCGGAAGUCCAAUCUGAAG
CCCUUCGAGCGGGACAUCUCCACCGAGAUCUAUCAGGCCGGCAGC
ACCCCUUGUAACGGCGUGGAAGGCUUCAACUGCUACUUCCCACUG
CAGUCCUACGGCUUUCAGCCCACAAAUGGCGUGGGCUAUCAGCCC
UACAGAGUGGUGGUGCUGAGCUUCGAACUGCUGCAUGCCCCUGCC
ACAGUGUGCGGCCCUAAGAAAAGCACCAAUCUCGUGAAGAACAAA
UGCGUGAACUUCAACUUCAACGGCCUGACCGGCACCGGCGUGCUG
ACAGAGAGCAACAAGAAGUUCCUGCCAUUCCAGCAGUUUGGCCGG
GAUAUCGCCGAUACCACAGACGCCGUUAGAGAUCCCCAGACACUG
GAAAUCCUGGACAUCACCCCUUGCAGCUUCGGCGGAGUGUCUGUG
AUCACCCCUGGCACCAACACCAGCAAUCAGGUGGCAGUGCUGUAC
CAGGACGUGAACUGUACCGAAGUGCCCGUGGCCAUUCACGCCGAU
CAGCUGACACCUACAUGGCGGGUGUACUCCACCGGCAGCAAUGUG
UUUCAGACCAGAGCCGGCUGUCUGAUCGGAGCCGAGCACGUGAAC
AAUAGCUACGAGUGCGACAUCCCCAUCGGCGCUGGAAUCUGCGCC
AGCUACCAGACACAGACAAACAGCCCUCGGAGAGCCAGAAGCGUG
GCCAGCCAGAGCAUCAUUGCCUACACAAUGUCUCUGGGCGCCGAG
AACAGCGUGGCCUACUCCAACAACUCUAUCGCUAUCCCCACCAAC
UUCACCAUCAGCGUGACCACAGAGAUCCUGCCUGUGUCCAUGACC
AAGACCAGCGUGGACUGCACCAUGUACAUCUGCGGCGAUUCCACC
GAGUGCUCCAACCUGCUGCUGCAGUACGGCAGCUUCUGCACCCAG
CUGAAUAGAGCCCUGACAGGGAUCGCCGUGGAACAGGACAAGAAC
ACCCAAGAGGUGUUCGCCCAAGUGAAGCAGAUCUACAAGACCCCU
CCUAUCAAGGACUUCGGCGGCUUCAAUUUCAGCCAGAUUCUGCCC
GAUCCUAGCAAGCCCAGCAAGCGGAGCUUCAUCGAGGACCUGCUG
UUCAACAAAGUGACACUGGCCGACGCCGGCUUCAUCAAGCAGUAU
GGCGAUUGUCUGGGCGACAUUGCCGCCAGGGAUCUGAUUUGCGCC
CAGAAGUUUAACGGACUGACAGUGCUGCCUCCUCUGCUGACCGAU
GAGAUGAUCGCCCAGUACACAUCUGCCCUGCUGGCCGGCACAAUC
ACAAGCGGCUGGACAUUUGGAGCAGGCGCCGCUCUGCAGAUCCCC
UUUGCUAUGCAGAUGGCCUACCGGUUCAACGGCAUCGGAGUGACC
CAGAAUGUGCUGUACGAGAACCAGAAGCUGAUCGCCAACCAGUUC
AACAGCGCCAUCGGCAAGAUCCAGGACAGCCUGAGCAGCACAGCA
AGCGCCCUGGGAAAGCUGCAGGACGUGGUCAACCAGAAUGCCCAG
GCACUGAACACCCUGGUCAAGCAGCUGUCCUCCAACUUCGGCGCC
AUCAGCUCUGUGCUGAACGAUAUCCUGAGCAGACUGGACCCUCCU
GAGGCCGAGGUGCAGAUCGACAGACUGAUCACAGGCAGACUGCAG
AGCCUCCAGACAUACGUGACCCAGCAGCUGAUCAGAGCCGCCGAG
AUUAGAGCCUCUGCCAAUCUGGCCGCCACCAAGAUGUCUGAGUGU
GUGCUGGGCCAGAGCAAGAGAGUGGACUUUUGCGGCAAGGGCUAC
CACCUGAUGAGCUUCCCUCAGUCUGCCCCUCACGGCGUGGUGUUU
CUGCACGUGACAUAUGUGCCCGCUCAAGAGAAGAAUUUCACCACC
GCUCCAGCCAUCUGCCACGACGGCAAAGCCCACUUUCCUAGAGAA
GGCGUGUUCGUGUCCAACGGCACCCAUUGGUUCGUGACACAGCGG
AACUUCUACGAGCCCCAGAUCAUCACCACCGACAACACCUUCGUG
UCUGGCAACUGCGACGUCGUGAUCGGCAUUGUGAACAAUACCGUG
UACGACCCUCUGCAGCCCGAGCUGGACAGCUUCAAAGAGGAACUG
GACAAGUACUUUAAGAACCACACAAGCCCCGACGUGGACCUGGGC
GAUAUCAGCGGAAUCAAUGCCAGCGUCGUGAACAUCCAGAAAGAG
AUCGACCGGCUGAACGAGGUGGCCAAGAAUCUGAACGAGAGCCUG
AUCGACCUGCAAGAACUGGGGAAGUACGAGCAGUACAUCAAGUGG
CCCUGGUACAUCUGGCUGGGCUUUAUCGCCGGACUGAUUGCCAUC
GUGAUGGUCACAAUCAUGCUGUGUUGCAUGACCAGCUGCUGUAGC
UGCCUGAAGGGCUGUUGUAGCUGUGGCAGCUGCUGCAAGUUCGAC
GAGGACGAUUCUGAGCCCGUGCUGAAGGGCGUGAAACUGCACUAC
ACAUGAUGACUCGAGCUGGUACUGCAUGCACGCAAUGCUAGCUGC
CCCUUUCCCGUCCUGGGUACCCCGAGUCUCCCCCGACCUCGGGUC
CCAGGUAUGCUCCCACCUCCACCUGCCCCACUCACCACCUCUGCU
AGUUCCAGACACCUCCCAAGCACGCAGCAAUGCAGCUCAAAACGC
UUAGCCUAGCCACACCCCCACGGGAAACAGCAGUGAUUAACCUUU
AGCAAUAAACGAAAGUUUAACUAAGCUAUACUAACCCCAGGGUUG
GUCAAUUUCGUGCCAGCCACACCCUGGAGCUAGCAGCGGCCGCGG
CCGCAAAAAAAAAAAAAAAAAAAAAAAAAAAAAAAAAAAAAAAAA
AAAAAAAAAAAAAAAAAAAAAAAAAAAAAAAAAAAAAAAAAAAAA
AAAAAAAAAAAAAAAAAAAAAAAAAAAAAAAAAAAAAAAAAAAA.

### Example 23. TLR9 CpG-ODN Adjuvant Sequences

Table 54 shows representative TLR9 CPG-ODN sequences; this list is not meant to be exhaustive. Underlined sequences indicate palindromes. Capital letters indicate phosphodiester linkages, lower case letters indicate phosphorothioate linkages. CpG-ODN 2006 is also referred to as ODN 7909 or PF3512676. In SEQ ID NO:10, N are nucleosides, x=0-3, y=1-4, w=−2, −1, 0, 1 or 2, p=0 or 1, q=0, 1 or 2, and z=1-20, X1 and X1′ are self-complementary nucleosides, X2 and X2′ are self-complementary nucleosides, and wherein the 5′ T of the (TCG(Nq))y sequence is 0-3 bases from the 5′ end of the polynucleotide; and (b) a palindromic sequence at least 8 bases in length wherein the palindromic sequence comprises the first (X1X2CGX2′X1′) of the (X1X2CGX2′X1′(CG)p)z sequences. In some embodiments, X1 and X2 are each either A or T.

TABLE 54
Representative TLR9 CpG-ODN adjuvants
and sequences

| SEQ | | |
|---|---|---|
| ID | | |
| NO | Description | Sequence |
| 2 | CpG-ODN 1018. Class B | 5′-tgactgtgaac |
| | | gttcgagatga-3′ |
| 3 | CpG-ODN 2006. Class B | 5′-tcgtcgttttg |
| | | tcgttttgtcgtt-3′ |
| 4 | CpG-ODN 1585. Class A | 5′-ggGGTCAACGT |
| | | TGAgggggG-3′ |
| 5 | CpG-ODN 1668. Class B | 5′-tccatgacgtt |
| | | cctgatgct-3′ |
| 6 | CpG-ODN 2216. Class A | 5′-ggGGGACGA: |
| | | TCGTCgggggg-3′ |
| 7 | CpG-ODN 2336. Class A | 5′-gggGACGAC: |
| | | GTCGTGgggggg-3′ |
| 8 | CpG-ODN 2395. Class C | 5′-tcgtcgttttc |
| | | ggcgc:gcgccg-3′ |
| 9 | CpG-ODN 10103. Class B | 5′-tcgtcgttttt |
| | | cggtcgtttt-3′ |
| 10 | CpG-ODN 7909 | 5′-Nx(TCG(Nq))y |
| | | Nw(X1X2CGX2′X1′ |
| | | (CG)p)z |

### Example 24. Sequences of Representative TLR 7/8 Agonist Uridine Rich Oligoribonucleotides

Table 55 contains a non-exhaustive list of oligoribonucleotides that bind and cause TLR7 and/or TLR8 downstream signaling.

TABLE 55
Representative TLR 7/8 uridine rich
oligoribonucleotide sequences

| SEQ | | |
|---|---|---|
| ID | | |
| NO | Description | Sequence |
| 11 | R-1075 | CCGUCUGUUGUGUGACUC |
| | R-1211 | dNdNdNdNdNGUUGUGUd |
| | | NdNdNdNdNdN |
| | R-1212 | dNdNdNdNdNdNUUGUGd |
| | | NdNdNdNdNdNdN |
| | R-1213 | dNdNdNdNdNdNUUGUdNd |
| | | NdNdNdNdNdNdN |
| 15 | R-1312 | GCCACCGAGCGUUGUGUACC |
| 16 | R-1321 | GCCACCGAGCAUUGUGAACC |
| 17 | R-1322 | GCCACCGAGCAUUGAGAACC |
| 18 | R-1323 | GCCACCGAGCAAUGAGAACC |
| 19 | R-1324 | GCCACCGAGCAAGGUGAACC |
| 20 | R-0001 | UAUAUAUAUAUAUAUAUAUA |
| 21 | R-0002 | UUAUUAUUAUUAUUAUUAUU |
| 22 | R-0003 | UUUAUUUAUUUAUUUAUUUA |
| 23 | R-0004 | UUUUAUUUUAUUUUAUUUUA |
| 24 | R-0005 | UGUGUGUGUGUGUGUGUGUG |
| 25 | R-0006 | UUGUUGUUGUUGUUGUUGUU |
| 26 | R-0007 | UUUGUUUGUUUGUUUGUUUG |
| 27 | R-0008 | UUUUGUUUUGUUUUGUUUUG |
| 28 | ORN 1 UUGU 4-mer | CCGAGCCGCUUGUCCC |
| 29 | ORN 1 UUUC 4-mer | CCGAGCCGCUUUCCCC |
| 30 | ORN 1 UTUT 4-mer | CCGAGCCGCUTUTCCC |
| 31 | ORN 1 UGUU 4-mer | CCGAGCCGCUGUUCCC |
| 32 | ORN 1 CUGU 4-mer | CCGAGCCGCCUGUCCC |
| 33 | ORN 1 UAUU 4-mer | CCGAGCCGCUAUUCCC |
| 34 | ORN 1 UUUT 4-mer | CCGAGCCGCUUUTCCC |
| 35 | ORN 1 TUGU 4-mer | CCGAGCCGCTUGUCCC |
| 36 | ORN 1 GUUU 4-mer | CCGAGCCGCGUUUCCC |
| 37 | ORN 1 AUUU 4-mer | CCGAGCCGCAUUUCCC |
| 38 | ORN 1 AUGU 4-mer | CCGAGCCGCAUGUCCC |
| 39 | ORN 1 CUUU 4-mer | CCGAGCCGCCUUUCCC |
| 40 | ORN 1 UUUA 4-mer | CCGAGCCGCUUUACCC |
| 41 | ORN 1 UUGC 4-mer | CCGAGCCGCUUGCCCC |
| 42 | ORN 1 UCUU 4-mer | CCGAGCCGCUCUUCCC |
| 43 | ORN 1 TUUC 4-mer | CCGAGCCGCTUUCCCC |
| 44 | ORN 1 AUUC 4-mer | CCGAGCCGCAUUCCCC |
| 45 | ORN 1 UUCT 4-mer | CCGAGCCGCUUCTCCC |
| 46 | ORN 1 UCUC 4-mer | CCGAGCCGCUCUCCCC |
| 47 | ORN 1 TUUU 4-mer | CCGAGCCGCTUUUCCC |
| 48 | ORN 1 UUGA 4-mer | CCGAGCCGCUUGACCC |
| 49 | ORN 1 UAUA 4-mer | CCGAGCCGCUAUACCC |
| 50 | ORN 1 GUUC 4-mer | CCGAGCCGCGUUCCCC |
| 51 | ORN 1 AUAU 4-mer | CCGAGCCGCAUAUCCC |
| 52 | ORN 1 AUAC 4-mer | CCGAGCCGCAUACCCC |
| 53 | ORN 1 UAUC 4-mer | CCGAGCCGCUAUCCCC |
| 54 | ORN 1 GCUC 4-mer | CCGAGCCGCGCUCCCC |
| 55 | ORN 1 CUAC 4-mer | CCGAGCCGCCUACCCC |
| 56 | ORN 1 GUAC 4-mer | CCGAGCCGCGUACCCC |
| 57 | ORN 1 GUGC 4-mer | CCGAGCCGCGUGCCCC |
| 58 | ORN 1 CUGC 4-mer | CCGAGCCGCCUGCCCC |
| 59 | ORN 1 UUTU 4-mer | CCGAGCCGCUUTUCCC |
| 60 | ORN 1 UUCU 4-mer | CCGAGCCGCUUCUCCC |
| 61 | ORN 1 AUAA 4-mer | CCGAGCCGCAUAACCC |
| 62 | ORN 1 CUAA 4-mer | CCGAGCCGCCUAACCC |
| 63 | ORN 1 UUUU 4-mer | CCGAGCCGCUUUUCCC |
| 64 | ORN 1 AUUA 4-mer | CCGAGCCGCAUUACCC |
| 65 | ORN 1 UUIU 4-mer | CCGAGCCGCUUIUCCC |
| 66 | ORN 1 UUAU 4-mer | CCGAGCCGCUUAUCCC |
| 67 | ORN 1 CUCC 4-mer | CCGAGCCGCCUCCCCC |
| 68 | ORN 1 AUCC 4-mer | CCGAGCCGCAUCCCCC |
| 69 | ORN 1 GUCC 4-mer | CCGAGCCGCGUCCCCC |
| 70 | ORN 2 UUGU 4-mer | CCGAGCCGAUUGUACC |
| 71 | ORN 2 UUUC 4-mer | CCGAGCCGAUUUCACC |
| 72 | ORN 2 UTUT 4-mer | CCGAGCCGAUTUTACC |
| 73 | ORN 2 UGUU 4-mer | CCGAGCCGAUGUUACC |
| 74 | ORN 2 CUGU 4-mer | CCGAGCCGACUGUACC |
| 75 | ORN 2 UAUU 4-mer | CCGAGCCGAUAUUACC |
| 76 | ORN 2 UUUT 4-mer | CCGAGCCGAUUUTACC |
| 77 | ORN 2 TUGU 4-mer | CCGAGCCGATUGUACC |
| 78 | ORN 2 GUUU 4-mer | CCGAGCCGAGUUUACC |
| 79 | ORN 2 AUUU 4-mer | CCGAGCCGAAUUUACC |
| 80 | ORN 2 AUGU 4-mer | CCGAGCCGAAUGUACC |
| 81 | ORN 2 CUUU 4-mer | CCGAGCCGACUUUACC |
| 82 | ORN 2 UUUA 4-mer | CCGAGCCGAUUUAACC |
| 83 | ORN 2 UUGC 4-mer | CCGAGCCGAUUGCACC |
| 84 | ORN 2 UCUU 4-mer | CCGAGCCGAUCUUACC |
| 85 | ORN 2 TUUC 4-mer | CCGAGCCGATUUCACC |
| 86 | ORN 2 AUUC 4-mer | CCGAGCCGAAUUCACC |
| 87 | ORN 2 UUCT 4-mer | CCGAGCCGAUUCTACC |
| 88 | ORN 2 UCUC 4-mer | CCGAGCCGAUCUCACC |
| 89 | ORN 2 TUUU 4-mer | CCGAGCCGATUUUACC |
| 90 | ORN 2 UUGA 4-mer | CCGAGCCGAUUGAACC |
| 91 | ORN 2 UAUA 4-mer | CCGAGCCGAUAUAACC |
| 92 | ORN 2 GUUC 4-mer | CCGAGCCGAGUUCACC |
| 93 | ORN 2 AUAU 4-mer | CCGAGCCGAAUAUACC |
| 94 | ORN 2 AUAC 4-mer | CCGAGCCGAAUACACC |
| 95 | ORN 2 UAUC 4-mer | CCGAGCCGAUAUCACC |
| 96 | ORN 2 GCUC 4-mer | CCGAGCCGAGCUCACC |
| 97 | ORN 2 CUAC 4-mer | CCGAGCCGACUACACC |
| 98 | ORN 2 GUAC 4-mer | CCGAGCCGAGUACACC |
| 99 | ORN 2 GUGC 4-mer | CCGAGCCGAGUGCACC |
| 100 | ORN 2 CUGC 4-mer | CCGAGCCGACUGCACC |
| 101 | ORN 2 UUTU 4-mer | CCGAGCCGAUUTUACC |
| 102 | ORN 2 UUCU 4-mer | CCGAGCCGAUUCUACC |
| 103 | ORN 2 AUAA 4-mer | CCGAGCCGAAUAAACC |
| 104 | ORN 2 CUAA 4-mer | CCGAGCCGACUAAACC |
| 105 | ORN 2 UUUU 4-mer | CCGAGCCGAUUUUACC |
| 106 | ORN 2 AUUA 4-mer | CCGAGCCGAAUUAACC |
| 107 | ORN 2 UUIU 4-mer | CCGAGCCGAUUIUACC |
| 108 | ORN 2 UUAU 4-mer | CCGAGCCGAUUAUACC |
| 109 | ORN 2 CUCC 4-mer | CCGAGCCGACUCCACC |
| 110 | ORN 2 AUCC 4-mer | CCGAGCCGAAUCCACC |
| 111 | ORN 2 GUCC 4-mer | CCGAGCCGAGUCCACC |
| 112 | 9.2dr | UGUCCUUCAAUGUCCUUCAA |
| 113 | 9.3as | UGGUAAUUGAAGGACAGGU |
| 114 | 9.2s | AGCUUAACCUGUCCUUCAA |
| 115 | US8658607B2 | GUUGUGGUUGUGGUUGUG |
| 116 | HIV sequence 1 | GUAGUGUGUG |
| 117 | HIV sequence 2 | GUCUGUUGUGUG |

TLR7/8 agonist nucleotide sequences. Phosphodiester or phosphorothioate linkages can be used. d, deoxyribonucleotides. Source for 9.2dr and 9.3as sequences: Bourquin C et al. J Immunol. 2009 Nov. 15; 183(10):6078-86. PMID: 19890064. Source for 9.2s: Hornung V et al. Nat Med. 2005 March; 11(3):263-70. PMID: 15723075. HIV sequence 1 and 2 are from U.S. Pat. No. 8,658,607B2. All other sequences are from Forsbach A et al. J Immunol. 2008 Mar. 15; 180(6):3729-38. PMID: 18322178.

### Example 25. Exemplary Phosphatidyl-L-Serine and Phosphatidylglycerol Targeted LNP Adjuvant and Antigen Combinations

Additional formulations shown below could also be prepared using the methods described in the Examples above. All lipid concentrations are shown as mol % as a percentage of the total lipid in the LNP. The below compositions vary in conjugated lipid content from 1.5 to 4 mol %, in sterol content from 25-37 mol %, in ICL content from 48-54 mol %, in saturated phosphatidyl-L-serine content from 2.5-7.5 mol %, and in total noncationic phospholipid content from 12-25 mol %. Most compositions would contain two phospholipids, typically phosphatidyl-L-serine (DSPS or DPPS being preferred) or phosphatidylglycerol (e.g. DSPG) and phosphatidylcholine, although some exemplary formulations may contain more than two phospholipids. LNPs may contain mRNA or other nucleic acid at N/P of 5-7. These LNP compositions containing oligonucleotide adjuvants of SEQ ID NOs: 2-126 can be combined with protein antigens, in either a soluble or nanoparticle anchored format. Listed in Table 57 is a non-exhaustive list of recombinant protein subunit vaccine antigens and hapten antigens (e.g. heroin and fentanyl haptens) that require or would likely require co-administration with an adjuvant in order to generate protective and durable immunity. Antigen valency is a critical parameter of immunogenicity: antigen multivalency increases crosslinking of B cell receptor molecules that potentiates downstream signaling and B cell activation that generally increases antibody titers, and improved uptake of antigen that results in a higher density of peptide/MHC molecules presented on the cell surface of antigen presenting cells to T cells (Kato Y et al. Immunity. 2020 Sep. 15; 53(3):548-563.e8. PMID: 3285795; Nguyen B, Tolia N H. NPJ Vaccines. 2021 May 13; 6(1):70. PMID: 33986287). Examples of potential nanoparticles antigen presenting formats include virus-like particles, encapsulation into carriers (e.g. liposomes), genetic fusion of antigen with self-assembling multimerization domains (e.g. foldon, ferritin, lumazine synthase), chemical conjugation of antigen to a self-assembling protein nanoparticle, and conjugation of tagged antigens with protein nanoparticles displaying a tag receptor/catcher (Nguyen B, Tolia N H. NPJ Vaccines. 2021 May 13; 6(1):70. PMID: 33986287). Table 58 shows examples of multimerization domains that have been fused to vaccine antigens to create self-assembling oligomers.

TABLE 56

| LNP | | SEQ ID |
|---|---|---|
| Formulation | Adjuvant | NO: |
| PS/KC3-OA | CpG 1018 (5′-TGACTGTGAA | 2 |
| LNP1 | CGTTCGAGATGA-3′) with | |
| | phosphorothioate linkages | |
| PG/KC3-OA | CpG 2006 (5′-TCGTCGTTTTG | 3 |
| LNP2 | TCGTTTTGTCGTT-3′) | |
| | with phosphorothioate linkages | |

TABLE 57
Examples of vaccine antigens requiring co-administration of adjuvant

| | | |
|---|---|---|
| infectious | | Reference |
| pathogen/disease | vaccine antigen description | (PMID) |

Viral

| | | |
|---|---|---|
| human VZV | envelope glycoprotein E (gE) | 25916341 |
| RSV | RSVpreF (DS-Cav1) and subsequent iterative structural | 24179220, |
| | improvements and nanoparticle scaffolds. For instance, | 27478931, |
| | sc9-10 DS-Cav1 A149C Y458C is a more stable version | 30849373, |
| | of DS-Cav1. DS-Cav1-I53-50A is an icosahedral | 37689061 |
| | scaffold that displays 20 trimeric complexes. | |
| HIV | gp120 eOD-GT-based immunogens (engineered outer | 23539181, |
| | domain, germline-targeting) | 31515132 |
| | Examples of 60mers: eOD-GT5, eOD-GT6, eOD-GT8 | |
| | Example of eOD-GT nanoparticle: eOD-GT8 fused to | |
| | lumazine synthase | |
| HIV | BG505 SOSIP Env trimer and related proteins (e.g. | 25892233, |
| | BG505 DS-SOSIP, BG505 DS-SOSIP.Dgly4), Env-gp41 | 27174988, |
| | fusion peptide, fusion peptide (e.g. FP8-KLH | 29867235 |
| | immunogen) | |
| HIV | conserved protease cleavage sites | 32853182 |
| human influenza | Quadrivalent inactivated virus (QIV). Split virus | approved |
| virus | | vaccines such as |
| | | Fluarix QIV |
| | | (GSK), Fluzone |
| | | QIV (Sanofi) |
| human influenza | HA stabilized stem nanoparticles. H1ssF, H3ssF, H7ssF, | 26301691, |
| A virus (IAV) | H10ssF | 26303961, |
| | | 30808695, |
| | | 36356572 |
| human influenza | neuraminidase | 35869085, |
| virus | | 35383176, |
| | | 34809451 |
| Lassa virus | stabilized LASV GPC | 38177144 |
| SARS-CoV-2 | Spike glycoprotein (2P) | 32075877 |
| MERS-CoV | MERS-CoV prefusion spike | 28807998 |
| Ebola virus | glycoprotein (GP), nucleoprotein (NP). For example, the | 32418798, |
| (EBOV) | antigen in the approved Ervebo vaccine. | 31589088, |
| | | 32234486 |
| Marburg | glycoprotein (GP), nucleoprotein (NP) | 31589088, |
| (MARV) | | 32234486 |
| HMPV | Human metapneumovirus F glycoprotein | 34551978 |
| parainfluenza | prefusion stabilized fusion proteins (e.g. prefusion PIV3 | 30420505 |
| virus type 1 and | F) | |
| 4 | | |

Bacterial

| | | |
|---|---|---|
| Mycobacterium | For example, M72 fusion protein (Mtb32A and Mtb39A) | U.S. Pat. No. |
| tuberculosis | of the vaccine candidate M72/AS01E | 10,639,361B2, |
| | | SEQ ID NO: 4 |
| | | (SEQ ID NO: 170) |

Protozoan

| | | |
|---|---|---|
| Plasmodium | RTS, S/AS01, R21/Matrix-M | 27355532, |
| falciparim | | 33964223 |
| (malaria) | | |

Fungal

| | | |
|---|---|---|
| Blastomyces | Blastomyces endoglucanase 2 (Bl-Eng2) | 34399628 |
| dermatitidis | | |

non-infectious

| | | |
|---|---|---|
| opioids | heroin and fentanyl hapten (CRM) | 33301675, |
| | | 37758302 |
| Alzheimer's | AV-1959R (Ab) and AV-1980R (tau) | 31847886 |
| Alzheimer's | AV-1980R/A fused with MultiTEP platform | 31664089 |

SEQ ID NO: 170:
GARVQRVVGSAPAASLGISTGDVITAVDGAPINSATAMADALNGHH
PGDVISVTWQTKSGGTRTGNVTLAEGPPAEFMVDFGALPPEINSA
RMYAGPGSASLVAAAQMWDSVASDLFSAASAFQSVVWGLTVGSWI
GSSAGLMVAAASPYVAWMSVTAGQAELTAAQVRVAAAAYETAYGL
TVPPPVIAENRAELMILIATNLLGQNTPAIAVNEAEYGEMWAQDA
AAMFGYAAATATATATLLPFEEAPEMTSAGGLLEQAAAVEEASDT
AAANQLMNNVPQALQQLAQPTQGTTPSSKLGGLWKTVSPHRSPIS
NMVSMANNHMSMTNSGVSMTNTLSSMLKGFAPAAAAQAVQTAAQN
GVRAMSSLGSSLGSSGLGGGVAANLGRAASVGSLSVPQAWAAANQ
AVTPAARALPLTSLTSAAERGPGQMLGGLPVGQMGARAGGGLSGV
LRVPPRPVMPHSPAAGDIAPPALSQDRFADFPALPLDPSAMVAQV
GPQVVNINTKLGYNNAVGAGTGIVIDPNGVVLTNNHVIAGATDIN
AFSVGSGQTYGVDVVGYDRTQDVAVLQLRGAGGLPSAAIGGGVAV
GEPVVAMGNSGGQGGTPRAVPGRVVALGQTVQASDSLTGAEETLN
GLIQFDAAIQPGDAGGPVVNGLGQVVGMNTAAS

TABLE 58
Examples of multimerization domains that can be genetically fused
to vaccine antigens to create self-assembling nanoparticles.

| Name | Size | # of monomer subunits |
|---|---|---|
| IMX313 | 4.4 | 7 |
| Nsp10 | 8.4 | 12 |
| T3 | 11 | 12 |
| T32 | 11 | 24 |
| Ferritin | 12 | 24 |
| T33 | 12.2 | 24 |
| O3 | 13 | 24 |
| lumazine synthase | 15 | 60 |
| T3 + 2 | 16 | 12 |
| O3 + 4CC | 18 | 24 |
| HbsAg VLP | 22 | ~96 |
| O3 + 2 | 22.5 | 24 |
| E2p | 23.2 | 60 |
| I3 | 25 | 60 |
| I3 + 5CC | 25 | 60 |
| I52 | 25.3 | 120 |
| AP205 VLP | 27.2 | 180 |
| I53 | 27.5 | 120 |
| I32 | 28.6 | 120 |
| Ico532-1 | 30 | 60 |

### Example 26. Exemplary Sequences of Synthetically-Produced Double-Stranded RNA that Bind to and Activated RIG-I Signaling

RIG-I is a cytosolic PRR that evolved to recognize infection by RNA viruses and RNA intermediates from microbial DNA. It recognizes blunt-ended double-stranded RNA (dsRNA) with an uncapped 5′ triphosphate or 5′ diphosphate that has an unmethylated 5′ terminal nucleotide (Schlee M et al. Immunity. 2009 Jul. 17; 31(1):25-34. PMID: 19576794; Goubau D et al. Nature. 2014 Oct. 16; 514(7522):372-375. PMID: 25119032). Additionally, some viral genomic 5′ triphosphate ssRNA with uridine- or adenine-rich stretches are recognized by RIG-I (Saito T et al. Nature. 2008 Jul. 24; 454(7203):523-7. PMID: 18548002; Schnell G et al. PLoS Pathog. 2012; 8(8):e1002839. PMID: 22912574). Using synthetic dsRNA, it has been demonstrated that while it is critical that the 5′ triphosphate end of the dsRNA be blunt, overhangs are acceptable on the opposing end within limits. For example, if the 5′ triphosphate strand contains 24 nucleotides, the anti-sense strand can be 23-20 nucleotides and still maintain activity. Anti-sense strands of less than 20 nucleotides may still maintain activity but it may be diminished or inactive (Schlee M et al. Immunity. 2009 Jul. 17; 31(1):25-34. PMID: 19576794). Activation of RIG-I results in downstream activation of the transcription factors IRF-3, IRF-7 and NFxB. This induces antiviral programs that include immunomodulatory cytokines and chemokines. These factors cumulatively shape the downstream innate and adaptive immune responses that are critical for pathogen clearance. Due to the natural activation of RIG-I by pathogenic viruses such as influenza A virus, hepatitis C virus, dengue, herpesviruses, coronaviruses (e.g. SARS-CoV-1 and SARS-CoV-2) and Zika viruses. RIG-I agonists have been shown to improve vaccine-induced immunity and allow for lower doses; consequently, RIG-I agonists such as poly(I:C) and the small molecule benzobisthiazole KIN1148 are promising vaccine adjuvant candidates (Hemann E A et al. J Immunol. 2023 May 1; 210(9):1247-1256. PMID: 36939421; Rehwinkel J et al. Nat Rev Immunol. 2020 September; 20(9):537-551. PMID: 32203325). As discussed earlier, translation of poly(I:C) to a vaccine adjuvant has been hindered by toxicity caused by off-target TLR7/8 and RIG-I activation. A similar toxicity profile is likely to exist for KIN1148, and the therapeutic index of these agonists could be improved with targeted nanoparticle delivery.

Table 60 contains single-stranded RNA sequences that activate the RIG-I signaling pathway when produced using in vitro transcription (IVT). Notably, IVT of single-stranded RNA templates can produce unintended side products consisting of double-stranded RNA and complementary single-stranded RNAs that anneal with the sense strand to form dsRNA, and these can bind to and activate RIG-I (Schlee M et al. Immunity. 2009 Jul. 17; 31(1):25-34. doi: 10.1016/j.immuni.2009.05.008PMID: 19576794).

Table 61 contains a non-exhaustive list of synthetic double-stranded RNA sequences consisting of an uncapped 5′ triphosphate (or diphosphate) sense strand paired with anti-sense strands of various lengths with a 5′ hydroxyl terminus. At a minimum, the 5′ terminal nucleotide of the triphosphate strand must be unmethylated in the 2′ position.

TABLE 60
Sequences of 5′ triphosphate oligoribonucleotides
derived from hepatitis C virus that bind and
activate RIG-I.

| SEQ ID | | |
|---|---|---|
| NO | Description | Sequence |
| 118 | poly-U/UG | UAAUACGACUCACUAUAGGGUUGGUUGGUUUUUUUUUGGG |
| | | UUUUUUUUUUUGUUUUUUUUUUUUUUUUUUUUUUUUUUUU |
| | | UUUUUUGUGGUUUUUUUUUGGUGUUUUUUUGGUUUUGUUU |
| | | GGUUU |
| 119 | poly-U/UC | UAAUACGACUCACUAUAGGCCAUCCUGUUUUUUUCCCUUUU |
| | | UUUUUUUCUUUUUUUUUUUUUUUUUUUUUUUUUUUUUUUU |
| | | UUCUCCUUUUUUUUUCCUCUUUUUUUCCUUUUCUUUCCUUU |
| 120 | poly-A/AG | UAAUACGACUCACUAUAGGGAAGGAAGGAAAAAAAAAGGG |
| | | AAAAAAAAAAGAAAAAAAAAAAAAAAAAAAAAAAAAAAAA |
| | | AAAGAGGAAAAAAAAAGGAGAAAAAAAGGAAAAGAAAGGA |
| | | AA |
| 121 | poly-U/UC 50 | UAAUACGACUCACUAUAGGCCAUCCUGUUUUUUUCCCUUUU |
| | | UUUUUUUCUUUUUUUUUUUUUUUUUU |
| 122 | poly-U/UC 25 | UAAUACGACUCACUAUAGGCCAUCCUGUUUUUUUCCCUUUU |
| | | U |
| 123 | replication | UAAUACGACUCACUAUAGGGAAAGGAAAGAAAAGGAAAAA |
| | intermediate | AAGAGGAAAAAAAAAGGAGAAAAAAAAAAAAAAAAAAAAA |
| | poly U/UC | AAAAAAAAAAAAAGAAAAAAAAAAAGGGAAAAAAACAGGA |
| | | UGGCC |
| 124 | 3′ NTR | UAAUACGACUCACUAUAGGGACUUGAUCUGCAGAGAGGCCA |
| | | G |
| 125 | 5′ NTR | UAAUACGACUCACUAUAGGGCCAGCCCCCGAUUGGGGGCGA |
| 126 | poly A | UAAUACGACUCACUAUGGGAAAAAAAAAAAAAAAAAAAAA |
| | | AAAAAAAAAAAAAAAAAAAAAAAAAAAAAAAAAAAAAAAA |
| | | AAAAAAAAAAAAAAAAAAAAAAAAAAAAAAAAAAAAA |

All oligoribonucleotides have 5′ triphosphate and are produced using in vitro transcription. Sources: Saito T et al. Nature. 2008 Jul. 24; 454(7203):523-7. PMID: 18548002. Schnell G et al. PLoS Pathog. 2012; 8(8):e1002839. PMGD: 22912574.

TABLE 61
Sequences of synthetic 5′ triphosphate dsRNA that bind and activate RIG-I
signaling. The table also includes examples of viruses whose genomes
contain panhandle structures that form blunt 5′ triphosphate
dsRNA structures. 3P indicates sense strands with a 5′
triphosphate.

| SEQ ID | | |
|---|---|---|
| NO | Description | Sequence or GenBank identifier |
| 127 | 3P-GFP2 | GACGCUGACCCUGAAGUUCAUCUU |
| 128 | 3P-GFP2 anti-sense (24mer) | AAGAUGAACUUCAGGGUCAGCGUC |
| 129 | 3P-GFP2 anti-sense (23mer) | AGAUGAACUUCAGGGUCAGCGUC |
| 130 | 3P-A24 | AACACACACACACACACACACUUU |
| 131 | anti-sense A21 (ass. with 3P-A24) | GUGUGUGUGUGUGUGUGUGUU |
| 132 | anti-sense A22 (ass. with 3P-A24) | AGUGUGUGUGUGUGUGUGUGUU |
| 133 | anti-sense A23 (ass. with 3P-A24) | AAGUGUGUGUGUGUGUGUGUGUU |
| 134 | anti-sense A24 (ass. with 3P-A24) | AAAGUGUGUGUGUGUGUGUGUGU |
| | | U |
| 135 | anti-sense A24+A (ass. with 3P- | AAAAGUGUGUGUGUGUGUGUGUG |
| | A24) | UU |
| 136 | anti-sense A24+2A (ass. with 3P- | AAAAAGUGUGUGUGUGUGUGUGU |
| | A24) | GUU |
| 137 | 3P-G24 | GACACACACACACACACACACUUU |
| 138 | anti-sense G21 (ass. with 3P-G24) | GUGUGUGUGUGUGUGUGUGUC |
| 139 | anti-sense G22 (ass. with 3P-G24) | AGUGUGUGUGUGUGUGUGUGUC |
| 140 | anti-sense G23 (ass. with 3P-G24) | AAGUGUGUGUGUGUGUGUGUGUC |
| 141 | anti-sense G24 (ass. with 3P-G24) | AAAGUGUGUGUGUGUGUGUGUGU |
| | | C |
| 142 | anti-sense G24+A (ass. with 3P- | AAAAGUGUGUGUGUGUGUGUGUG |
| | G24) | UC |
| 143 | anti-sense G24+2A (ass. with 3P- | AAAAAGUGUGUGUGUGUGUGUGU |
| | G24) | GUC |
| 144 | 3P-C24 | CACACACACACACACACACACUUU |
| 145 | anti-sense C21 (ass. with 3P-C24) | GUGUGUGUGUGUGUGUGUGUG |
| 146 | anti-sense C22 (ass. with 3P-C24) | AGUGUGUGUGUGUGUGUGUGUG |
| 147 | anti-sense C23 (ass. with 3P-C24) | AAGUGUGUGUGUGUGUGUGUGUG |
| 148 | anti-sense C24 (ass. with 3P-C24) | AAAGUGUGUGUGUGUGUGUGUGU |
| | | G |
| 149 | anti-sense C24+A (ass. with 3P- | AAAAGUGUGUGUGUGUGUGUGUG |
| | C24) | UG |
| 150 | anti-sense C24+2A (ass. with 3P- | AAAAAGUGUGUGUGUGUGUGUGU |
| | C24) | GUG |
| 151 | 3P-U24 | UACACACACACACACACACACUUU |
| 152 | anti-sense U21 (ass. with 3P-U24) | GUGUGUGUGUGUGUGUGUGUA |
| 153 | anti-sense U22 (ass. with 3P-U24) | AGUGUGUGUGUGUGUGUGUGUA |
| 154 | anti-sense U23 (ass. with 3P-U24) | AAGUGUGUGUGUGUGUGUGUGUA |
| 155 | anti-sense U24 (ass. with 3P-U24) | AAAGUGUGUGUGUGUGUGUGUGU |
| | | A |
| 156 | anti-sense U24+A (ass. with 3P- | AAAAGUGUGUGUGUGUGUGUGUG |
| | U24) | UA |
| 157 | anti-sense U24+2A (ass. with 3P- | AAAAAGUGUGUGUGUGUGUGUGU |
| | U24) | GUA |
| 158 | rabies panhandle | GACGCUUAACAAAUAAACAACAAA |
| | | AAUGAGAAAAACAAUCAUAUGUC |
| | | UGUUUUUUCUUUGAUCUGGUUGU |
| | | UAAGCGUC |
| 159 | influenza A, segment 5 | NC_002019 |
| 160 | rabies genome | NC_001542 |
| 161 | NDV genome | NC_002617 |
| 162 | VSV genome | NC_001560 |
| 163 | measles genome | NC_001498 |
| 164 | RSV genome | NC_001803 |
| 165 | Sendai genome | NC_001552 |
| 166 | 3P-G19 | GCAUGCGACCUCUGUUUGA |
| 167 | anti-sense G19 (ass. with 3P-G19) | CGUACGCUGGAGACAAACU |

Sources: Schlee M et al. Immunity. 2009 Jul. 17; 31(1):25-34. PMID: 19576794; Wang Y et al. Nat Struct Mol Biol. 2010 July; 17(7):781-7. PMID: 20581823; Hornung V et al. Science. 2006 Nov. 10; 314(5801):994-7. PMID: 17038590.

### Example 27. In Vivo Inflammatory Profile of CpG-ODN 1018 Delivered in Various LNP Formulations

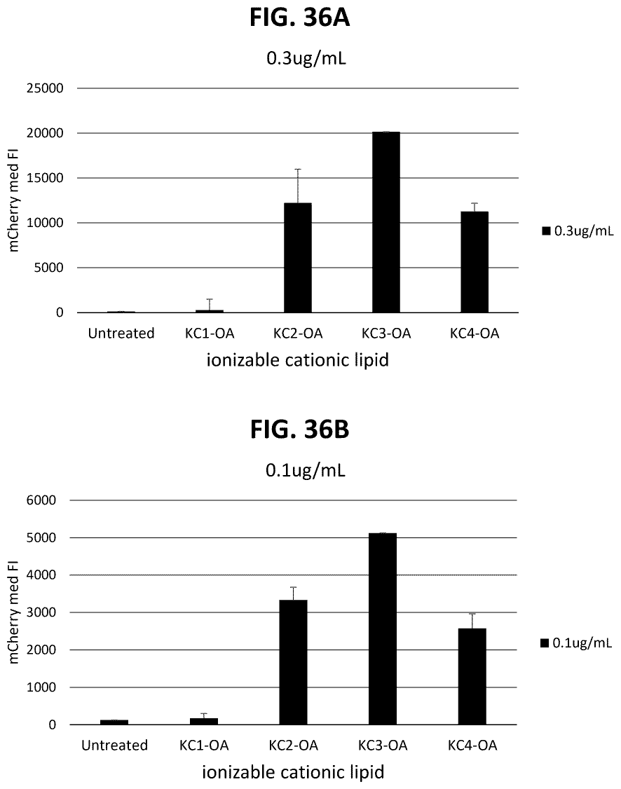

The primary aim of this study was to examine the effect of various ionizable cationic lipids (ICLs) on the delivery of oligonucleotide adjuvants to endosomal TLRs. Since TLR9 is located in the endosome, ICLs that are inefficient at mediating endosomal escape of mRNA and subsequent translation may be sufficient for endosomal delivery of oligonucleotides to TLR9. We have shown that the KC-OA family of ICLs with 2 or more methylenes in the head group (i.e. KC2-OA, KC3-OA and KC4-OA) are relatively efficient at mediating mRNA endosomal escape and translation compared to KC1-OA, which has a single methylene in the head group (FIGS. 36A-36B, Table 63). AKG-OA-DM2 (DM2) and AKG-OA-DM3 (DM3) are also relatively poor for mRNA transfection (Tables 64 and 65; compare fluorescence intensities to those in Table 63 using the same cytometer settings for KC2-OA, KC3-OA and KC4-OA). To this end, CpG-ODN 1018 was encapsulated in LNPs containing different ionizable cationic lipids while DSPC phospholipid, cholesterol and PEG-DMG were held constant. The study design is shown in Table 66. All LNPs were prepared with the based formulation of ICL/DSPC/DPPS-NH4/Chol/PEG-DMG (48/5/5/40.5/1.5 mol %). Nucleic acids were incorporated into LNPs at an N/P ratio of 5.25. All LNP exhibited greater than 90% CpG oligo entrapment and sizes ranged from 65-139 nm. All particles were stable to freezing and thawing at −80° C. as judged by the small changes in Z-Ave particle sizes after F/T (Table 67).

TABLE 62
Physicochemical properties and characterization of LNPs formulated
with the KC-OA family of ICLs and mCherry mRNA.

| | | Post-Filter | | Post F/T | | | | | |
|---|---|---|---|---|---|---|---|---|---|
| | nucleic | Z- | | Z- | | Post F/T | | zeta | |
| Formulation | acid | Average | PDI | Average | PDI | % EE | stdev | ph 5 | ph 7 |
| KC1-OA/PS | mCherry | 67.4 | 0.1 | 67.47 | 0.11 | 84.8 | 5.0 | 6.24 | −12.08 |
| KC2-OA/PS | | 82.8 | 0.05 | 81.55 | 0.05 | 87.7 | 3.5 | 14.59 | −4.39 |
| KC3-OA/PS | | 103.4 | 0.03 | 101.5 | 0.07 | 90.2 | 6.2 | 16.46 | −2.9 |
| KC4-OA/PS | | 115.8 | 0.12 | 116.6 | 0.09 | 92.2 | 4.9 | 18.06 | −0.08 |

% EE, oligonucleotide encapsulation efficiency; stdev, % EE standard deviation; post filter, after buffer exchange by centrifugal concentrators and 0.2-μm filtration; post F/T, after freeze-thaw cycle; Z-Ave, z-average particle size by DLS, nm; PDI, size polydispersity index. Zeta refers to zeta potential measurements at either pH 5 or pH 7 in units of mV.

TABLE 63
In vitro mCherry expression in MutuDC1940 cells 24 hrs post
transfection with LNPs formulated with KC-OA family ICLs.

| | 0.3 ug/mL | | 0.1 ug/mL | | Fold change relative | |
|---|---|---|---|---|---|---|
| | mCherry-LNP | | mCherry-LNP | | to KC1-OA | |
| Transfection | medFI | | medFI | | 0.3 | 0.1 |
| group | mean | stdev | mean | stdev | ug/mL | ug/mL |
| untreated | 123.0 | 2.0 | 123.0 | 2.0 | n/a | n/a |
| KC1-OA/PS | 282.7 | 6.8 | 175.3 | 4.6 | 1.0 | 1.0 |
| KC2-OA/PS | 12205.0 | 904.0 | 3335.0 | 383.5 | 43.2 | 19.0 |
| KC3-OA/PS | 20129.0 | 3571.4 | 5123.3 | 376.8 | 71.2 | 29.2 |
| KC4-OA/PS | 11263.7 | 1100.0 | 2575.7 | 192.9 | 39.8 | 14.7 |

Cells were transfected with either 0.3 or 0.1 ug/mL mRNA-LNP and mCherry expression was quantified 24 hrs later by flow cytometry.

TABLE 64
Physicochemical properties and characterization of LNPs formulated
with AKG-UO-1, AKG-OA-DM2 or AKG-OA-DM3 and mCherry mRNA.

| | | | Post-Filter | | Post F/T | | | |
|---|---|---|---|---|---|---|---|---|
| | mol % | nucleic | Z- | | Z- | | Post F/T | |
| Formulation | DSPS | acid | Average | PDI | Average | PDI | % EE | stdev |
| AKG-UO-1 | 0 | mCherry | 76.55 | 0.102 | N.D | N.D | 87.6 | 4.63 |
| (083021-1) | | | | | | | | |
| AKG-OA-DM2 | 7.5 | | 94.4 | 0.166 | 109.0 | 0.105 | 91.6 | 9.3 |
| (092321-7) | | | | | | | | |
| AKG-OA-DM3 | 7.5 | | 95.1 | 0.214 | 113.2 | 0.166 | 90.9 | 7.8 |
| (092321-10) | | | | | | | | |

% EE, oligonucleotide encapsulation efficiency; stdev, % EE standard deviation; post filter, after buffer exchange by centrifugal concentrators and 0.2-μm filtration; post F/T, after freeze-thaw cycle; Z-Ave, z-average particle size by DLS, nm; PDI, size polydispersity index. N.D is not determined.

TABLE 65
In vitro mCherry expression in MutuDC1940 cells 24 hrs post
transfection with LNPs formulated with AKG-UO-1, AKG-OA-DM2
or AKG-OA-DM3 ICLs. Cells were transfected with either 1, 0.33,
or 0.11 ug/mL mRNA-LNP and mCherry expression was quantified
24 hrs later by flow cytometry. For comparisons in transfection
efficiency, compare to median fluorescence intensities to those
in Table 63 using the same cytometer settings.

| | dose | mCherry | | Fold change |
|---|---|---|---|---|
| mRNA-LNP | (ug/mL) | medFI | stdev | over untreated |
| untreated | n/a | 291 | 13 | 1 |
| AKG-OA-DM2 | 1 | 1721 | 312 | 5.9 |
| 7.5 mol % DSPS | 0.33 | 590 | 59 | 2.0 |
| | 0.11 | 386 | 22 | 1.3 |
| AKG-OA-DM3 | 1 | 977 | 138 | 3.4 |
| 7.5 mol % DSPS | 0.33 | 489 | 29 | 1.7 |
| | 0.11 | 360 | 7 | 1.2 |
| AKG-UO-1 | 1 | 3287 | 697 | 11.3 |
| 0 mol % DSPS | 0.33 | 1029 | 85 | 3.5 |
| | 0.11 | 634 | 59 | 2.2 |

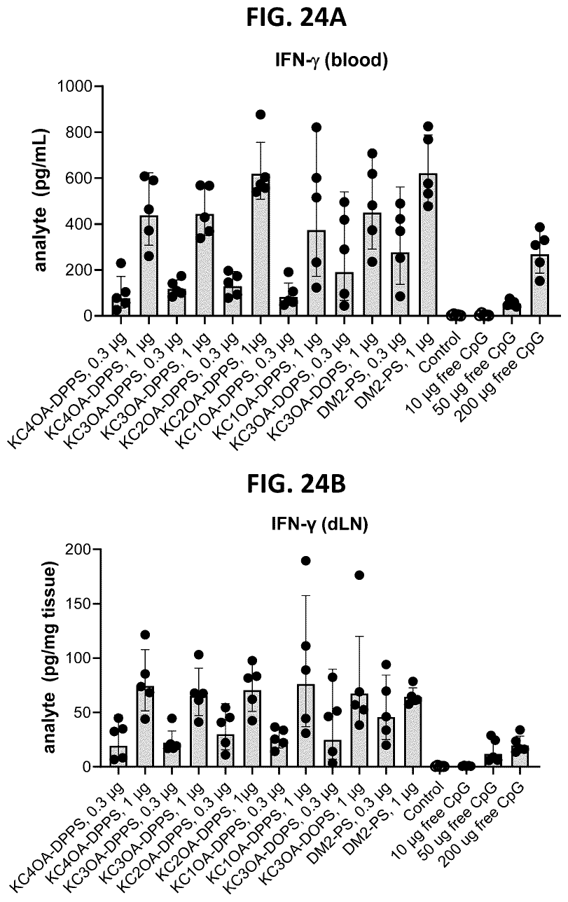

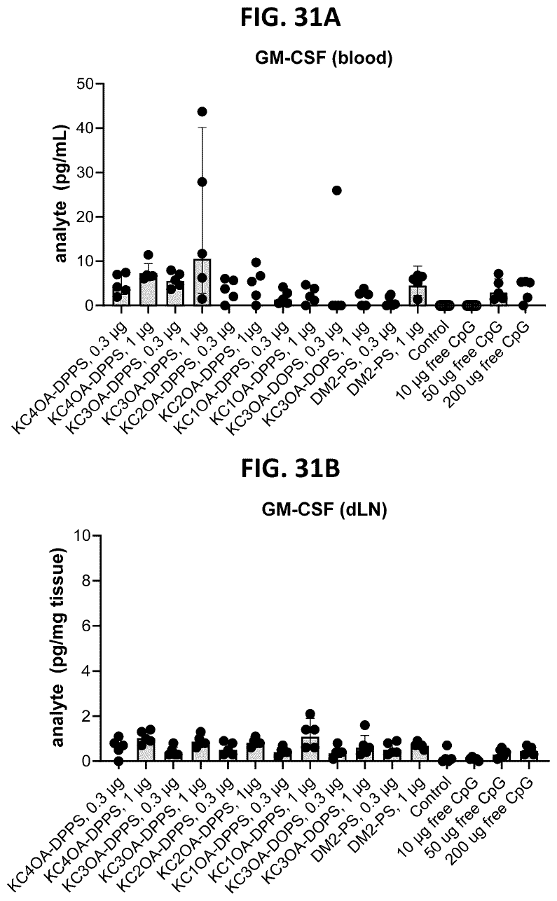

Blood and dLNs (pooled iliac, popliteal and inguinal LNs) were harvested 6 hrs post-injection. Serum and tissues were prepared as described in Example 21. Cytokines and chemokines were quantified using the LEGENDplex 13-plex Mouse Anti-Virus Response Panel (BioLegend, Cat. No. 740622). LNs were weighed prior to lysis, and analyte concentrations were converted from pg/mL to pg/mg of tissue mass. Results shown in FIGS. 24A-35B are shown as the geometric mean and geometric SD. The analyte concentrations in the blood serum are reported in Table 68 and the mean fold changes over vehicle treatment are shown in Table 69. The analyte concentrations in the dLNs are reported in Table 70 and the mean fold changes over vehicle treatment are shown in Table 71.

TABLE 66
Study design to examine in vivo inflammatory profiles of CpG-
ODNs delivered by LNPs with varying ICLs. All BALB/c mice except
for Group 1 received CpG-ODN 1018. Group 2-13 were administered
CpG-1018 encapsulated in PS-targeted LNPs. DPPS was used in
Groups 2-9 and DOPS was used in Groups 10 and 11. Groups 14-16
received non-encapsulated “naked” CpG-ODN 1018. Injections
were i.m. in the left caudal thigh in a volume of 20 μL.

| | | | Dose per mRNA |
|---|---|---|---|
| Group | CpG-ODN | LNP | (ug) |
| 1 | vehicle | n/a | n/a |
| 2 | CpG 1018 | KC4OA-DPPS | 0.3 |
| 3 | CpG 1018 | KC4OA-DPPS | 1 |
| 4 | CpG 1018 | KC3OA-DPPS | 0.3 |
| 5 | CpG 1018 | KC3OA-DPPS | 1 |
| 6 | CpG 1018 | KC2OA-DPPS | 0.3 |
| 7 | CpG 1018 | KC2OA-DPPS | 1 |
| 8 | CpG 1018 | KC1OA-DPPS | 0.3 |
| 9 | CpG 1018 | KC1OA-DPPS | 1 |
| 10 | CpG 1018 | KC3OA-DOPS | 0.3 |
| 11 | CpG 1018 | KC3OA-DOPS | 1 |
| 12 | CpG 1018 | DM2-DPPS | 0.3 |
| 13 | CpG 1018 | DM2-DPPS | 1 |
| 14 | CpG 1018 | n/a | 10 |
| 15 | CpG 1018 | n/a | 50 |
| 16 | CpG 1018 | n/a | 200 |

TABLE 67
Physicochemical properties and characterization of LNPs loaded with
CpG-ODN oligos that were used in Example 27, FIGS. 24A-35B.

| | | Particle | | | |
|---|---|---|---|---|---|
| | Particle | Size (nm) | | Zeta | Zeta |
| ICL | size (nm) | Post-F/T | E.E, % | pH 5 | pH 7 |
| KC4-OA/DPPS | 97.57 | 98.33 | 92.0 ± 2.2 | 21.05 | 1.959 |
| KC3-OA/DPPS | 95.31 | 95.45 | 90.9 ± 1.0 | 22.25 | −1.506 |
| KC2-OA/DPPS | 78.45 | 78.78 | 91.1 ± 0.9 | 14.15 | −4.07 |
| KC1-OA/DPPS | 65.17 | 64.93 | 91.4 ± 1.8 | 6.331 | −8.18 |
| OA-DM2/DPPS | 137.4 | 138.6 | 91.1 ± 0.9 | 27.87 | 4.142 |
| KC3-OA/DOPS | 104 | 104.3 | 90.7 ± 0.9 | 17.63 | 0.6418 |

% EE, oligonucleotide encapsulation efficiency; stdev, % EE standard deviation; post-mix, after mixing of the lipids and UUGU oligo solutions; post filter, after buffer exchange by centrifugal concentrators and 0.2-μm filtration; post F/T, after freeze-thaw cycle; Z-Ave, z-average particle size by DLS, nm; PDI, size polydispersity index. Zeta refers to zeta potential measurements at either pH 5 or pH 7 in units of mV.

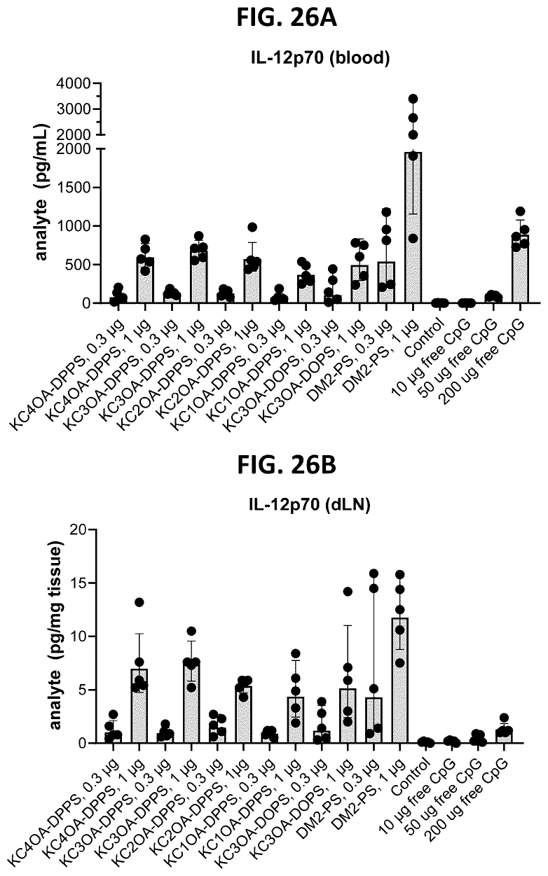

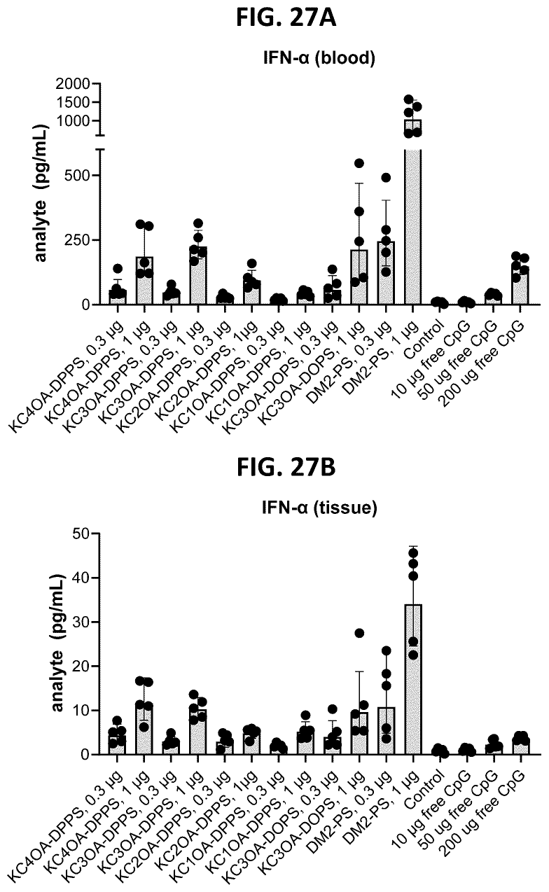

PS-targeted LNPs efficiently delivered CpG-ODN 1018 to TLR9-expressing innate immune cells at the lowest tested dose of 0.3 μg CpG, resulting in downstream signaling and upregulation of most analytes systemically in the blood and locally in the dLNs (FIGS. 24A-35B). Within the KC series of ionizable cationic lipids, KC4-OA, KC3-OA, KC2-OA and KC1-OA had similar biological activity. AKG-OA-DM2 was unique compared to the KC-series in that it specifically induced the highest levels of IL-12p70 (FIGS. 26A-26B) and type I IFN-α (FIGS. 27A-27B) in the blood and dLNs at the 1 μg dose; otherwise its proinflammatory profile was quantitatively similar to the other ICLs. It was unexpected that KC1-OA and AKG-OA-DM2 LNP formulations performed as well as LNPs comprised of either KC2-OA, KC3-OA or KC4-OA considering their relatively poor mRNA transfection efficiency. CpG delivered by OA-DM2-containing LNPs to endosomes may have unique pharmacokinetics from KC series ICLs that favors some cytokines, and depending on the vaccine and type of protective immunity, different ICLs may be preferential depending on the desired induction of cytokines such as IL-12p70 and IFN-α that are important in shaping adaptive immunity.

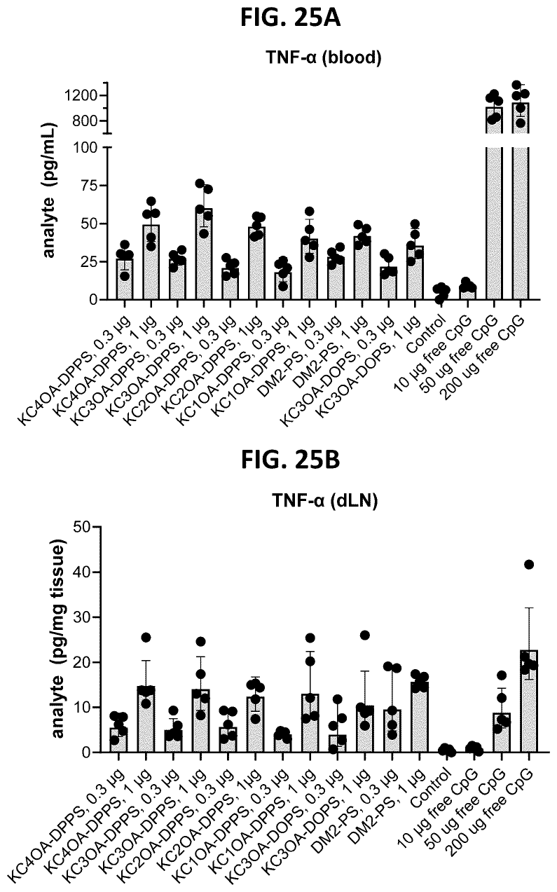

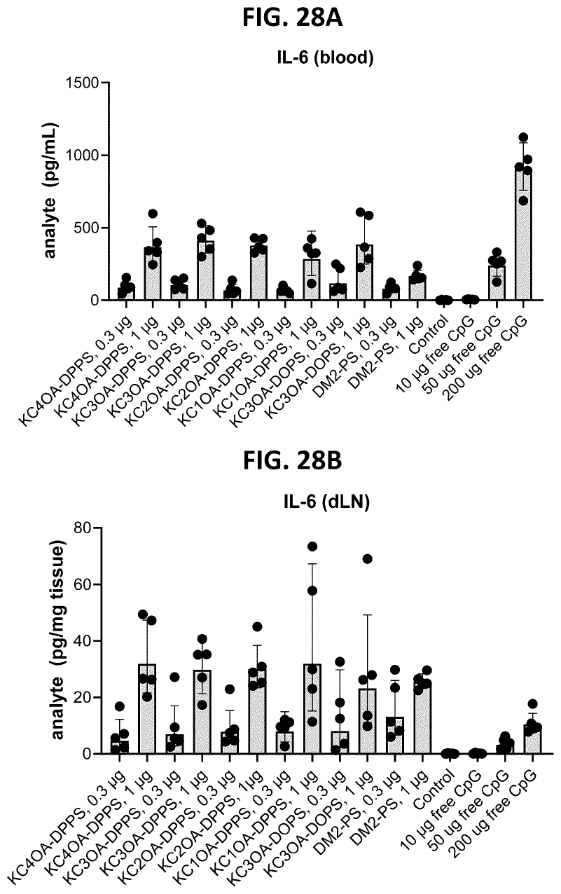

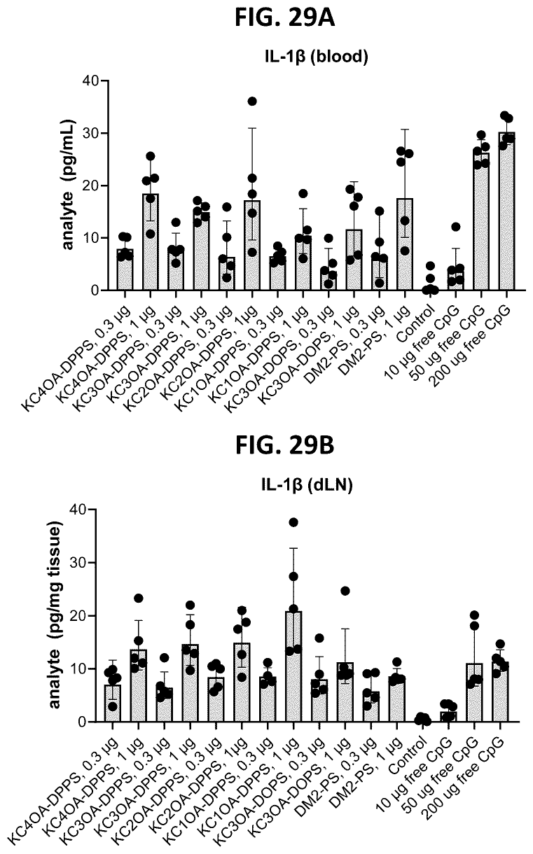

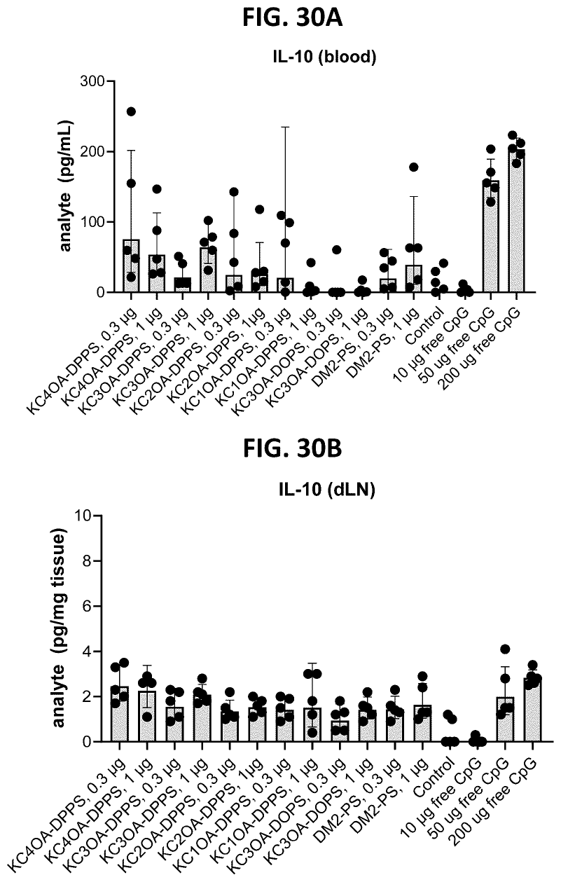

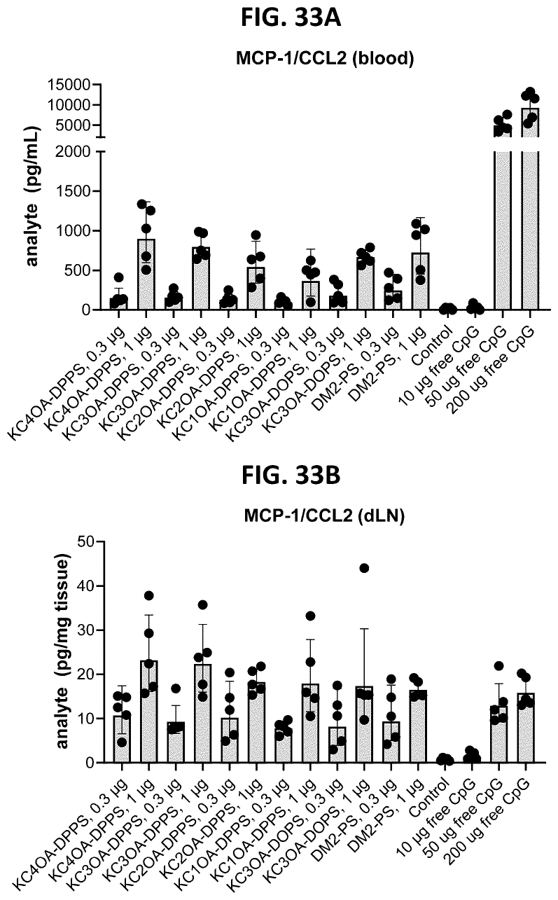

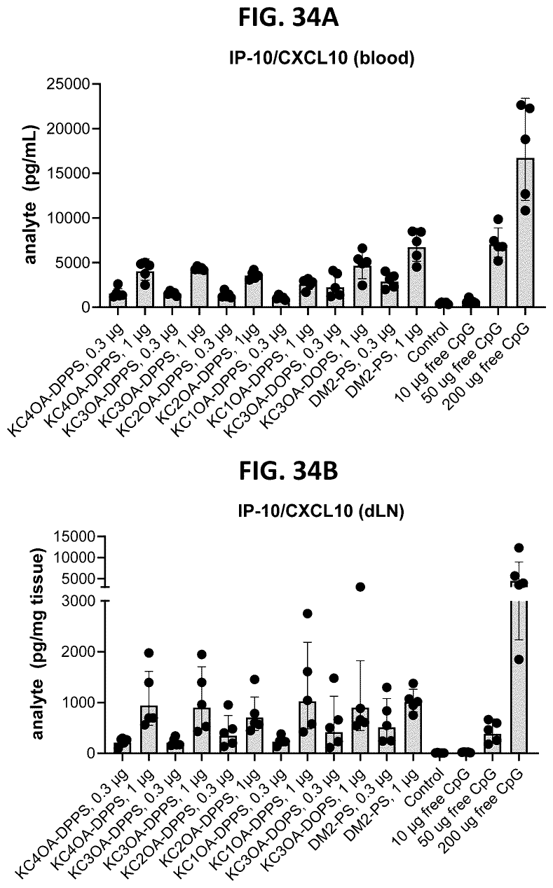

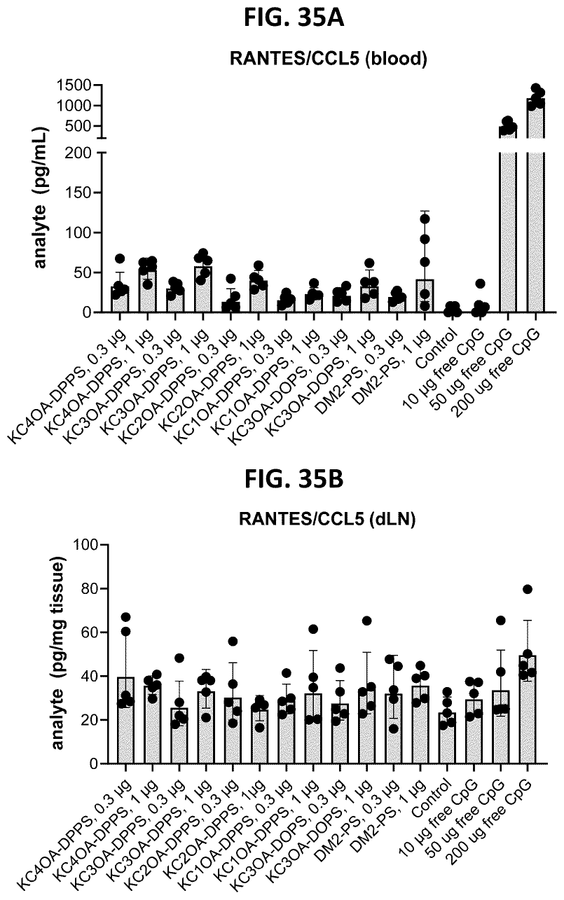

In contrast to CpG-LNPs, the inflammatory profile of unencapsulated “free” CpG-ODN 1018 was markedly different than delivery by LNPs. Injection of 10 μg free CpG-ODN 1018 did not elevate immunomodulatory cytokines/chemokines above baseline vehicle concentrations in the blood or dLNs for any of the analytes tested, demonstrating that CpG was inefficiently delivered and internalized by innate immune cells. At a minimum, delivery of LNP encapsulated CpG was >30-fold more efficient (activity at 0.3 g with LNPs, no measurable activity at 10 g unencapsulated). At the higher doses of 50 and 200 g, unencapsulated CpG was biologically active, but this modality favored the induction and systemic release of some analytes that was not observed with CpG-LNPs. Specifically, higher doses of unencapsulated CpG (50 and/or 200 g depending on the analyte) caused high systemic levels of TNF-α (FIG. 25A), IL-6 (FIG. 28A), IL-1β (FIG. 29A), IL-10 (FIG. 30A), KC (FIG. 32A), MCP-1/CCL2 (FIG. 33A), IP10/CXCL10 (FIG. 34A) and RANTES/CCL5 (FIG. 35A) in the blood. These elevated levels relative to LNP delivery did not occur locally in the dLNs (with the exception of CXCL10). The cytokines TNF-α, IL-6 and IL-1β are pleiotropic and induce the downstream production of many acute-phase proinflammatory cytokines and cause other physiological changes (Tarrant J M. Toxicol Sci. 2010 September; 117(1):4-16. PMID: 20447938; Tahtinen S et al. Nat Immunol. 2022 April; 23(4):532-542. PMID: 35332327; Norelli M et al. Nat Med. 2018 June; 24(6):739-748. PMID: 29808007; Hunter C A, Jones S A. Nat Immunol. 2015 May; 16(5):448-57. PMID: 25898198.). CCL2 and KC mobilize monocytes and neutrophils, respectively, to site of inflammation and especially monocytes have been shown to potent producers of IL-1β and IL-6.

As the B cell and T cell responses are primed in the local draining LN, it is desirable to maximize local immunomodulatory cytokines and minimize systemic release that are in excess and may not improve the adaptive immune response. Compared to the systemic upregulation of proinflammatory cytokines and chemokines caused by i.m. injection of unencapsulated CpG, delivery by LNPs promoted local release of analytes and at higher concentrations than free CpG. For instance, at the 1 g dose, CpG-LNPs were more efficient at inducing local IFN-γ (FIG. 24B), IL-12p70 (FIG. 26B), IFN-α (FIG. 26B), IL-6 (FIG. 28B) and KC (FIG. 32B) than a 200 μg dose of free CpG.

In conclusion, delivery of free CpG-ODNs requires high doses when used as a vaccine adjuvant. Delivery of CpG-ODN via LNPs efficiently targets innate immune cells in the draining LNs, induces acute immunomodulatory cytokines and chemokines that instruct the development of humoral and cellular immunity to vaccine antigens. Relative to free CpG, CpG-LNPs allow for reduced adjuvant dosing, induces a more focused, local acute inflammatory response within the dLN with lower systemic levels of proinflammatory cytokines, and qualitatively signal differently than free CpG to induce local IL-12p70 and type I IFNs expression. LNPs formulated with a variety of ionizable cationic lipids allows for efficient release of CpG-ODN in the endosomal compartment. Unexpectedly, some ICLs that are ineffective in mRNA-LNP formulations at promoting endosomal escape of mRNA into the cytosol are efficient in this setting.

### Example 28. In Vivo Inflammatory Profile of UUGU Oligoribonucleotides Delivered in LNP Formulations Using Different ICLs

The primary aim of this study was to examine the effect of various ionizable cationic lipids (ICLs) on the delivery of a UUGU oligoribonucleotide to TLR7/8 located intracellularly in endolysosomal compartments. We also sought to determine whether PS-targeting of LNPs influenced activity. Similar to the rationale in Example 27, ICLs that are inefficient at mRNA transfection in immune cells may be able to efficiently deliver UUGU oligos to TLR-7/8. UUGU oligos were encapsulated in LNPs formulated with KC-OA family ICLs, AKG-UO-DM2, DODMA, or DODAP. We also tested the ALC-0315 and SM-102 LNP formulations. The effect of PS targeting on biological activity was also assessed using either DOPS or DPPS formats.

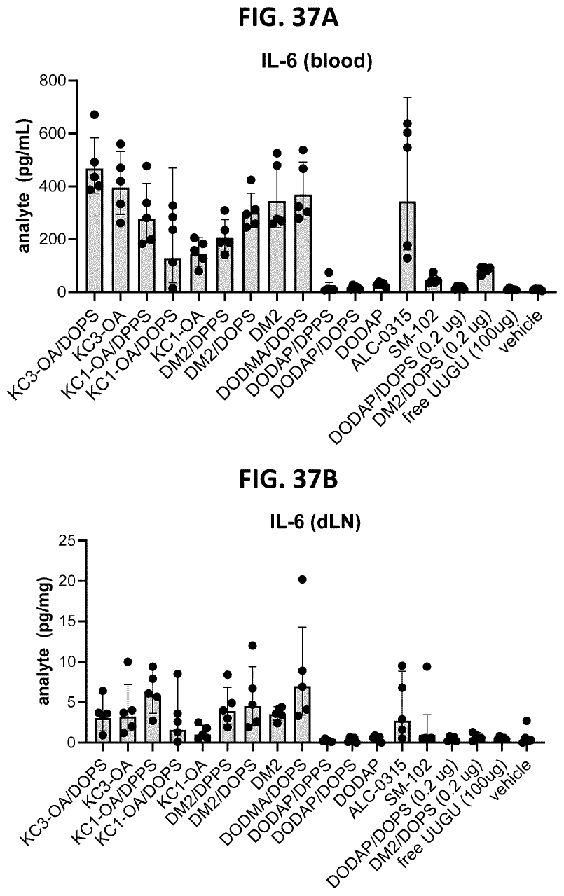

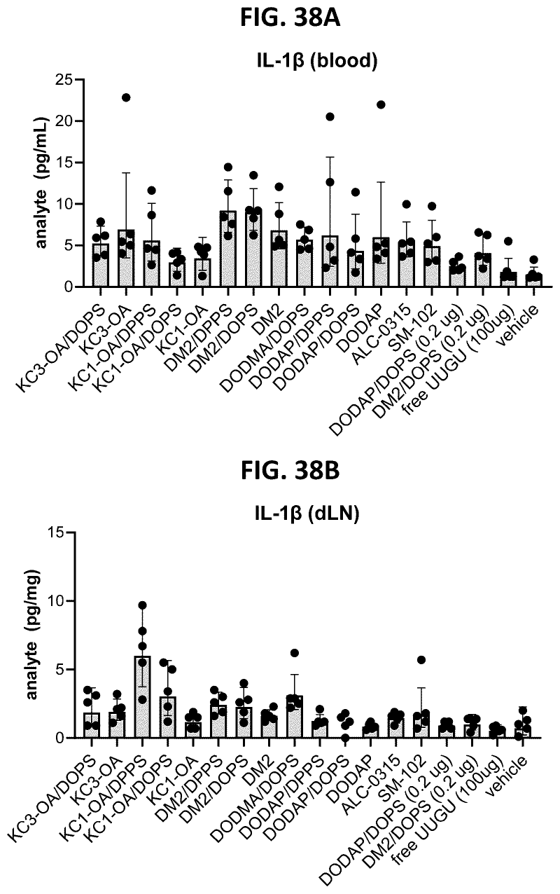

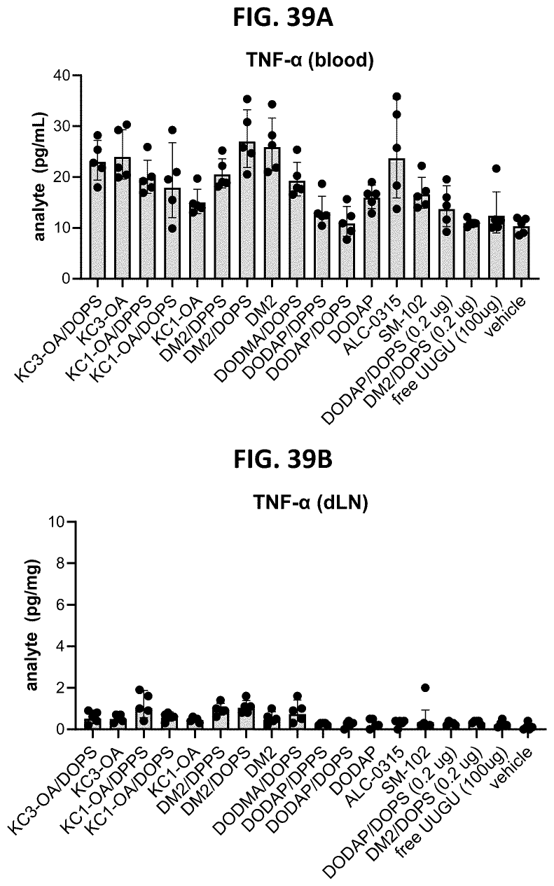

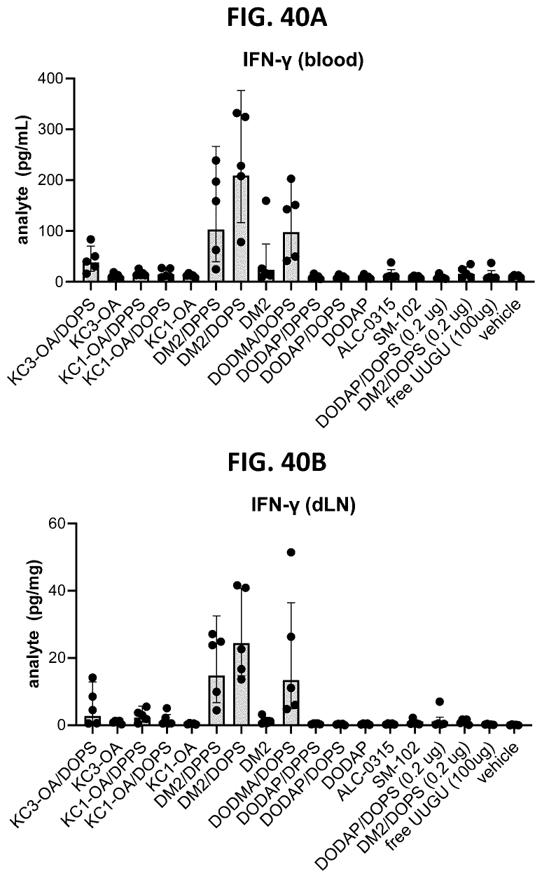

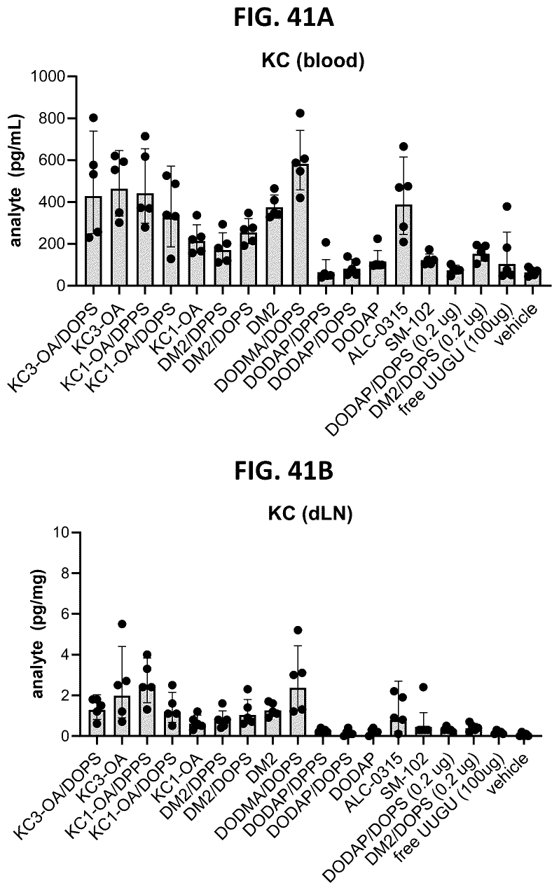

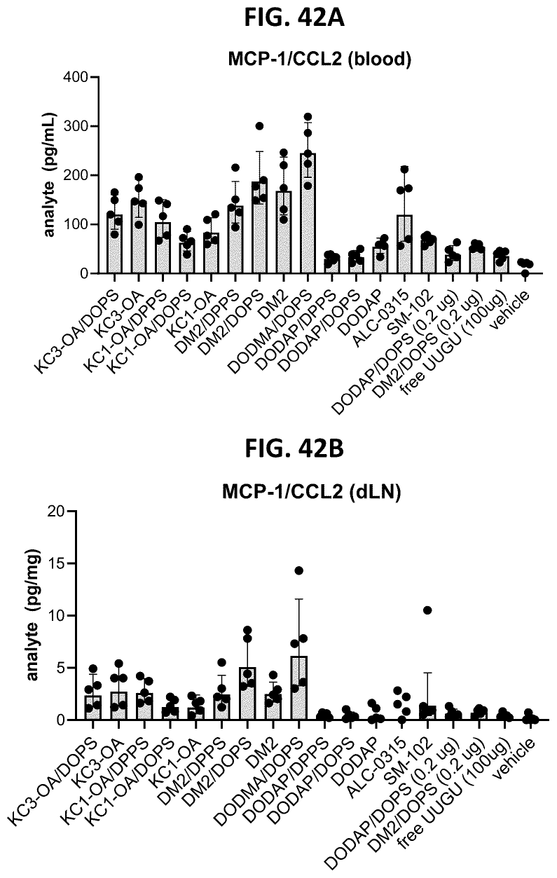

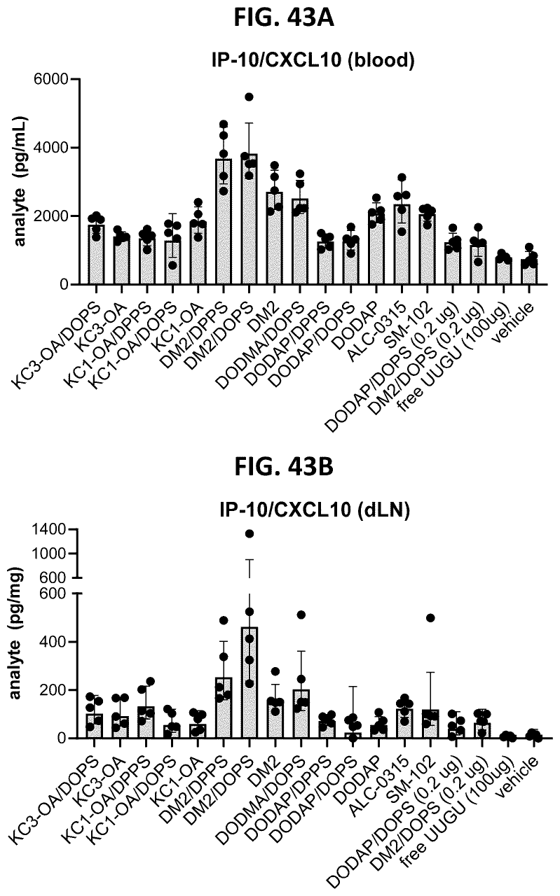

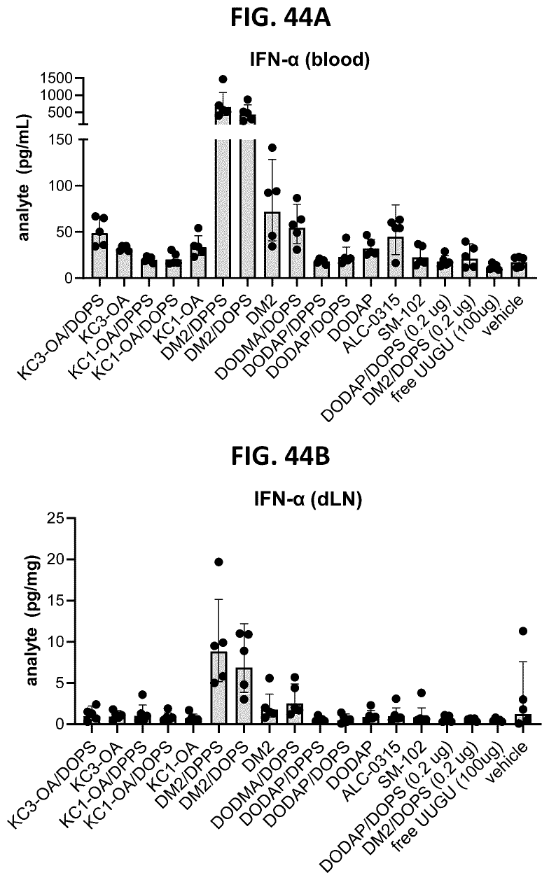

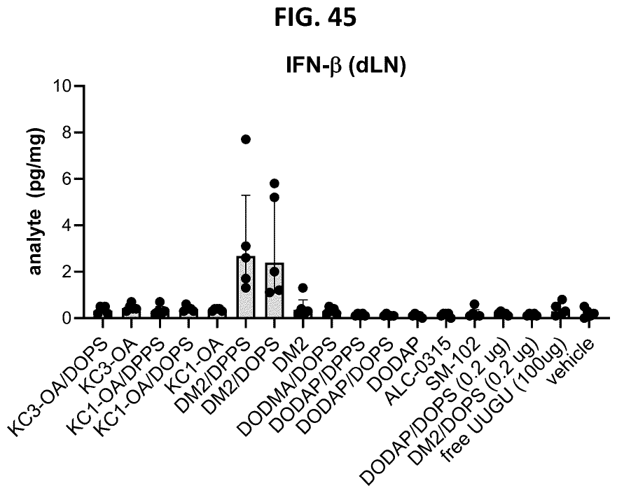

Unencapsulated UUGU ribooligonucleotide was highly inefficient at inducing expression of any of the 13 analytes, including type I IFN-α, even at a high dose of 100 ug. In contrast, LNPs formulated with multiple different ICLs were able to induce biological activity at a 1 ug UUGU dose (TABLES 72-75). In particular, KC1-OA, KC3-OA, AKG-UO-DM2 and DODMA ICLs were the most efficient at inducing a proinflammatory signature primarily consisting of IFN-g (FIGS. 40A-40B), IFN-α (FIGS. 44A-44B), IFN-b (FIG. 45), IL-6 (FIGS. 37A-37B), IL-1b (FIGS. 38A-38B), TNFα (FIGS. 39A-39B) KC/CCL5 (FIG. 41A-41B), MCP-1/CCL2 (FIGS. 42A-42B), and IP10/CXCL10 (FIGS. 43A-43B). In contrast to CpG-LNPs, UUGU was a poor inducer of IL-12p70 and TNF-α (TABLES 72-75). Of the ICLs tested, DM2 was the most potent LNP adjuvant formulation. ALC-0315 and SM-102 LNP formulations had relatively poor activity, and DODAP LNPs had the least activity. Importantly, both DOPS and DSPS targeting increased the efficiency of UUGU delivery to TLR7/8. Untargeted KC1-OA, KC3-OA, and DM2 induced levels of cytokines such as IFN-g, IFN-α, IFN-b and the chemokines KC/CCL5, MCP-1/CCL2, and IP-10/CXCL10. PS could be increasing UUGU activity by increasing LNP uptake and/or promoting LNP uptake by TLR7 expressing cell subsets such as pDCs and B cells.

TABLE 68
Concentration (pg/mL) of proinflammatory analytes in blood serum 6 hrs post i.m. injection of
CpG-based adjuvants. There were 5 animals per group and values are shown as the mean (stdev).

| | Dose | | | | | | |
|---|---|---|---|---|---|---|---|
| delivery | CpG-ODN | | | | | | |
| format | (μg) | IFN-g | | TNF-a | | IL-12p70 | |
| vehicle | n/a | 3.6 | (2.6) | 4.9 | (3.5) | 1.7 | (2.1) |
| KC4-OA/DPPS | 0.3 | 98.8 | (78.6) | 28 | (7.6) | 100.1 | (72) |
| | 1 | 458.8 | (147.3) | 50.7 | (12.3) | 611.2 | (158.4) |
| KC3-OA/DPPS | 0.3 | 121.6 | (36.3) | 27 | (4.4) | 132.2 | (33.7) |
| | 1 | 454.5 | (108.4) | 61.3 | (13.4) | 690.8 | (125.9) |
| KC2-OA/DPPS | 0.3 | 137.4 | (50.7) | 21.3 | (5) | 133.3 | (44.2) |
| | 1 | 630 | (140) | 48.3 | (6.3) | 598.3 | (221.4) |
| KC1-OA/DPPS | 0.3 | 94.4 | (58.1) | 19.2 | (6.7) | 81.6 | (61) |
| | 1 | 458.6 | (282) | 41.3 | (11.4) | 383 | (126.6) |
| KC3-OA/DOPS | 0.3 | 268.9 | (196.7) | 22.3 | (6.1) | 189.6 | (179.6) |
| | 1 | 483.1 | (188.3) | 36.6 | (9.9) | 544.6 | (241.6) |
| DM2/DPPS | 0.3 | 323.6 | (159.3) | 28.4 | (4.6) | 679.2 | (436.1) |
| | 1 | 636 | (152.5) | 42.2 | (5.7) | 2161.9 | (952.5) |
| unencapsulated | 10 | 4.6 | (4.6) | 9 | (1.8) | 0.9 | (1.3) |
| CpG | 50 | 54.7 | (14.1) | 1034 | (185.4) | 91.4 | (14.6) |
| | 200 | 281.9 | (90.9) | 1111.2 | (234.4) | 900.1 | (184.5) |

| delivery | | | | | | | | |
|---|---|---|---|---|---|---|---|---|
| format | IFN-a | | IL-6 | | IL-1β | | IL-10 | |
| vehicle | 9.3 | (4.1) | 1.8 | (2.5) | 1.5 | (2) | 18 | (17.3) |
| KC4-OA/DPPS | 65.7 | (42.2) | 95 | (41) | 8.1 | (1.9) | 108.2 | (97.2) |
| | 204 | (96.3) | 382.2 | (132) | 19.2 | (5.6) | 67.2 | (51) |
| KC3-OA/DPPS | 49.1 | (17.1) | 109.3 | (35.6) | 8.2 | (2.9) | 26 | (18.5) |
| | 231.1 | (57.1) | 418.9 | (93.4) | 15 | (1.6) | 68.6 | (26) |
| KC2-OA/DPPS | 29.4 | (8.1) | 74.6 | (38.3) | 7.8 | (5.3) | 56 | (58.4) |
| | 99.4 | (36.7) | 379.2 | (46.3) | 19.6 | (10.7) | 39.8 | (44.5) |
| KC1-OA/DPPS | 20.8 | (5) | 68.1 | (20.9) | 6.6 | (1.3) | 58.6 | (49.2) |
| | 43.5 | (10) | 309.9 | (116.1) | 11.1 | (4.6) | 10.9 | (17.9) |
| KC3-OA/DOPS | 68.4 | (44.4) | 138.2 | (88.9) | 4.6 | (3.3) | 12.1 | (27) |
| | 269 | (191.4) | 413.9 | (173.6) | 13.1 | (6.4) | 4.3 | (7.5) |
| DM2/DPPS | 271.4 | (137.1) | 84.7 | (28.8) | 7.6 | (5) | 29.6 | (22.8) |
| | 1101.9 | (415.2) | 171.3 | (39.6) | 19.6 | (8.7) | 65.9 | (67.7) |
| unencapsulated | 9.4 | (3.8) | 5.8 | (2) | 4.8 | (4.3) | 3.6 | (5) |
| CpG | 40.5 | (5.3) | 250.5 | (76) | 26.4 | (2.4) | 161.2 | (28.1) |
| | 151.5 | (34.4) | 918.5 | (157.7) | 30.3 | (2.6) | 203.7 | (15.4) |

| | Dose | | | | | | | | | | |
|---|---|---|---|---|---|---|---|---|---|---|---|
| delivery | CpG-ODN | | | | | | | | | | |
| format | (μg) | KC | | MCP-1 | | IP-10 | | RANTES | | GM-CSF | |
| vehicle | n/a | 38.9 | (13.2) | 14.6 | (14.2) | 439.8 | (108.9) | 4.1 | (4) | 0 | (0) |
| KC4-OA/DPPS | 0.3 | 239.1 | (57.1) | 177.3 | (133.2) | 1599.9 | (579.2) | 35.6 | (18.2) | 4.8 | (2.3) |
| | 1 | 653 | (353.1) | 960.3 | (360.1) | 4156.3 | (1056.9) | 54.2 | (11.9) | 7.5 | (2.2) |
| KC3-OA/DPPS | 0.3 | 301.1 | (125) | 165.6 | (67.2) | 1538.7 | (226.7) | 30.7 | (7) | 5.8 | (1.8) |
| | 1 | 751.5 | (297.1) | 808.2 | (160.7) | 4322.2 | (191.6) | 59.3 | (14.1) | 18.2 | (17.4) |
| KC2-OA/DPPS | 0.3 | 228.9 | (104.2) | 138.9 | (66.1) | 1369.4 | (367.3) | 17.5 | (15) | 3.5 | (2.5) |
| | 1 | 573.4 | (226.4) | 588.5 | (258.1) | 3575 | (448.9) | 41.2 | (11.6) | 4.8 | (3.8) |
| KC1-OA/DPPS | 0.3 | 233.3 | (19.8) | 104 | (39.2) | 1062.4 | (237) | 16.2 | (6.2) | 1.9 | (1.6) |
| | 1 | 544.7 | (151.6) | 428.7 | (196.7) | 2639.1 | (580.3) | 23.9 | (7.8) | 2.3 | (1.9) |
| KC3-OA/DOPS | 0.3 | 305.2 | (64) | 213.9 | (132.2) | 2526.9 | (1338.9) | 22.1 | (8.3) | 5.2 | (11.6) |
| | 1 | 777 | (206.2) | 672 | (88.2) | 4881.3 | (1514.3) | 35.8 | (17.1) | 1.8 | (1.7) |
| DM2/DPPS | 0.3 | 178.5 | (36.4) | 282.4 | (152) | 2981.2 | (845.6) | 20.1 | (5.7) | 1 | (1.2) |
| | 1 | 153.3 | (57.9) | 787 | (322.3) | 6924.2 | (1737.5) | 60.6 | (45.7) | 5.1 | (2.2) |
| unencapsulated | 10 | 57.6 | (15.6) | 30.3 | (34.4) | 653.4 | (262.6) | 10.4 | (14.9) | 0 | (0) |
| CpG | 50 | 1065.5 | (337.8) | 5161.9 | (1720.7) | 7208.1 | (1695.5) | 500.6 | (114.4) | 3.6 | (2.5) |
| | 200 | 2517.5 | (715.2) | 9861 | (3490.6) | 17448 | (5448) | 1190.3 | (185.8) | 3.5 | (2.5) |

TABLE 69
Fold change in mean serum analyte concentrations over vehicle
6 hrs post i.m. injection of CpG-based adjuvants.

| | Dose | | | | | | | |
|---|---|---|---|---|---|---|---|---|
| delivery | CpG-ODN | | | | | | | |
| format | (μg) | IFN-g | TNF-a | IL-12p70 | IFN-a | IL-6 | IL-1β | IL-10 |
| vehicle | n/a | 1 | 1 | 1 | 1 | 1 | 1 | 1 |
| KC4-OA/DPPS | 0.3 | 27.4 | 5.7 | 58.9 | 7.1 | 52.8 | 5.4 | 6 |
| | 1 | 127.4 | 10.3 | 359.5 | 21.9 | 212.3 | 12.8 | 3.7 |
| KC3-OA/DPPS | 0.3 | 33.8 | 5.5 | 77.8 | 5.3 | 60.7 | 5.5 | 1.4 |
| | 1 | 126.3 | 12.5 | 406.4 | 24.8 | 232.7 | 10 | 3.8 |
| KC2-OA/DPPS | 0.3 | 38.2 | 4.3 | 78.4 | 3.2 | 41.4 | 5.2 | 3.1 |
| | 1 | 175 | 9.9 | 351.9 | 10.7 | 210.7 | 13.1 | 2.2 |
| KC1-OA/DPPS | 0.3 | 26.2 | 3.9 | 48 | 2.2 | 37.8 | 4.4 | 3.3 |
| | 1 | 127.4 | 8.4 | 225.3 | 4.7 | 172.2 | 7.4 | 0.6 |
| KC3-OA/DOPS | 0.3 | 74.7 | 4.6 | 111.5 | 7.4 | 76.8 | 3.1 | 0.7 |
| | 1 | 134.2 | 7.5 | 320.4 | 28.9 | 229.9 | 8.7 | 0.2 |
| DM2/DPPS | 0.3 | 89.9 | 5.8 | 399.5 | 29.2 | 47.1 | 5.1 | 1.6 |
| | 1 | 176.7 | 8.6 | 1271.7 | 118.5 | 95.2 | 13.1 | 3.7 |
| unencapsulated | 10 | 1.3 | 1.8 | 0.5 | 1 | 3.2 | 3.2 | 0.2 |
| CpG | 50 | 15.2 | 211 | 53.8 | 4.4 | 139.2 | 17.6 | 9 |
| | 200 | 78.3 | 226.8 | 529.5 | 16.3 | 510.3 | 20.2 | 11.3 |

| | Dose | | | | |
|---|---|---|---|---|---|
| delivery | CpG-ODN | | | | |
| format | (μg) | KC | MCP-1 | IP-10 | RANTES |
| vehicle | n/a | 1 | 1 | 1 | 1 |
| KC4-OA/DPPS | 0.3 | 6.1 | 12.1 | 3.6 | 8.7 |
| | 1 | 16.8 | 65.8 | 9.5 | 13.2 |
| KC3-OA/DPPS | 0.3 | 7.7 | 11.3 | 3.5 | 7.5 |
| | 1 | 19.3 | 55.4 | 9.8 | 14.5 |
| KC2-OA/DPPS | 0.3 | 5.9 | 9.5 | 3.1 | 4.3 |
| | 1 | 14.7 | 40.3 | 8.1 | 10 |
| KC1-OA/DPPS | 0.3 | 6 | 7.1 | 2.4 | 4 |
| | 1 | 14 | 29.4 | 6 | 5.8 |
| KC3-OA/DOPS | 0.3 | 7.8 | 14.7 | 5.7 | 5.4 |
| | 1 | 20 | 46 | 11.1 | 8.7 |
| DM2/DPPS | 0.3 | 4.6 | 19.3 | 6.8 | 4.9 |
| | 1 | 3.9 | 53.9 | 15.7 | 14.8 |
| unencapsulated | 10 | 1.5 | 2.1 | 1.5 | 2.5 |
| CpG | 50 | 27.4 | 353.6 | 16.4 | 122.1 |
| | 200 | 64.7 | 675.4 | 39.7 | 290.3 |

The fold change was not calculated for IFN-β because the vehicle was below the level of detection.

TABLE 70
Concentration (pg/mg of tissue) of proinflammatory analytes in
draining lymph nodes 6 hrs post i.m. injection of adjuvants.

| | Dose | | | | | | | | |
|---|---|---|---|---|---|---|---|---|---|
| delivery | CpG-ODN | | | | | | | | |
| format | (μg) | IFN-g | | TNF-a | | IL-12p70 | | IFN-a | |
| vehicle | n/a | 0.6 | (0.5) | 0.5 | (0.4) | 0.1 | (0.1) | 0.9 | (0.5) |
| KC4-OA/DPPS | 0.3 | 25.4 | (17.1) | 6 | (2.3) | 1.3 | (0.9) | 4.7 | (2.1) |
| | 1 | 78.5 | (28.4) | 15.5 | (5.8) | 7.5 | (3.3) | 12.3 | (4.4) |
| KC3-OA/DPPS | 0.3 | 23.8 | (11.8) | 5.3 | (2.4) | 1 | (0.5) | 3.1 | (1.1) |
| | 1 | 68.2 | (22.4) | 15.1 | (6.3) | 7.6 | (1.9) | 10.5 | (2.4) |
| KC2-OA/DPPS | 0.3 | 34.7 | (17.8) | 6.3 | (2.9) | 1.7 | (0.8) | 3.3 | (1.5) |
| | 1 | 73.4 | (21.4) | 12.8 | (3.3) | 5.4 | (0.7) | 4.9 | (1.1) |
| KC1-OA/DPPS | 0.3 | 26.5 | (9) | 4.1 | (0.7) | 0.9 | (0.3) | 2 | (0.5) |
| | 1 | 93 | (63) | 14.7 | (7.9) | 4.9 | (2.5) | 5.5 | (2.1) |
| KC3-OA/DOPS | 0.3 | 39.5 | (31.5) | 5.8 | (4.3) | 1.8 | (1.5) | 4.8 | (3.3) |
| | 1 | 78.6 | (55.7) | 12 | (8) | 6.4 | (4.8) | 11.8 | (9.2) |
| DM2/DPPS | 0.3 | 52.7 | (29.5) | 11.4 | (7.1) | 7.6 | (7.2) | 13.4 | (8.4) |
| | 1 | 64.8 | (8.2) | 15.7 | (1.4) | 12.2 | (3.3) | 35.4 | (10.6) |
| unencapsulated | 10 | 0.9 | (0.3) | 1 | (0.5) | 0.2 | (0.1) | 1.1 | (0.4) |
| CpG | 50 | 14.9 | (11) | 9.7 | (4.9) | 0.4 | (0.4) | 2.4 | (1) |
| | 200 | 20.7 | (8.3) | 24 | (10) | 1.3 | (0.6) | 3.7 | (0.6) |

| delivery | | | | | | | |
|---|---|---|---|---|---|---|---|
| format | IL-6 | | IL-1β | | IL-10 | IFN-β | |
| vehicle | 0.1 | (0.1) | 0.6 | (0.4) | 0.4 (0.6) | 0 | (0) |
| KC4-OA/DPPS | 6.6 | (6.1) | 7.7 | (2.8) | 2.6 (0.8) | 0.9 | (1.2) |
| | 33.9 | (13.4) | 14.4 | (5.3) | 2.4 (0.7) | 4.2 | (1.3) |
| KC3-OA/DPPS | 9.8 | (10) | 6.9 | (3) | 1.7 (0.6) | 0.1 | (0.2) |
| | 31.1 | (9.1) | 15.3 | (4.9) | 2.1 (0.5) | 3.9 | (1.5) |
| KC2-OA/DPPS | 9.6 | (7.6) | 8.7 | (2.4) | 1.4 (0.5) | 0.3 | (0.6) |
| | 30.8 | (8.4) | 15.7 | (5.1) | 1.6 (0.4) | 1.5 | (0.8) |
| KC1-OA/DPPS | 8.9 | (3.8) | 8.7 | (1.6) | 1.5 (0.5) | 0 | (0) |
| | 39.1 | (25.7) | 22.7 | (10.2) | 1.9 (1.1) | 0.8 | (1.2) |
| KC3-OA/DOPS | 13.6 | (12.6) | 8.7 | (4.2) | 1.1 (0.5) | 1 | (1.8) |
| | 29.3 | (23.5) | 12.3 | (7) | 1.5 (0.5) | 4 | (5.1) |
| DM2/DPPS | 15.8 | (10.3) | 6.3 | (2.8) | 1.5 (0.5) | 6.4 | (4.3) |
| | 25.7 | (2.6) | 8.7 | (1.5) | 1.8 (0.8) | 15.2 | (5.9) |
| unencapsulated | 0.2 | (0.2) | 2.3 | (1.3) | 0.1 (0.1) | 0 | (0) |
| CpG | 3.7 | (2) | 12.3 | (6.3) | 2.2 (1.2) | 0 | (0) |
| | 10.9 | (3.9) | 11.5 | (2.1) | 2.8 (0.3) | 2.1 | (2.2) |

| | Dose | | | | | | | | | | |
|---|---|---|---|---|---|---|---|---|---|---|---|
| delivery | CpG-ODN | | | | | | | | | | |
| format | (μg) | KC | | MCP-1 | | IP-10 | | RANTES | | GM-CSF | |
| vehicle | n/a | 0.2 | (0.1) | 0.8 | (0.3) | 9.7 | (4) | 24.1 | (6.4) | 0.2 | (0.3) |
| KC4-OA/DPPS | 0.3 | 2.7 | (1.3) | 11.5 | (4.3) | 228.9 | (80.8) | 42.9 | (19.2) | 0.6 | (0.4) |
| | 1 | 10.4 | (4.2) | 24.5 | (9.2) | 1064.4 | (605.4) | 35.8 | (4.2) | 1 | (0.3) |
| KC3-OA/DPPS | 0.3 | 3 | (2.5) | 9.8 | (3.9) | 221.8 | (82.5) | 27.3 | (12.3) | 0.4 | (0.2) |
| | 1 | 12.2 | (6.1) | 23.4 | (8) | 1051.9 | (630.6) | 33.9 | (7.7) | 0.9 | (0.3) |
| KC2-OA/DPPS | 0.3 | 3.6 | (1.9) | 11.6 | (6.3) | 434.3 | (324.1) | 32.6 | (14.6) | 0.6 | (0.3) |
| | 1 | 9.7 | (2.1) | 18.4 | (2.7) | 769.7 | (401.5) | 25.2 | (5.1) | 0.8 | (0.2) |
| KC1-OA/DPPS | 0.3 | 3.3 | (1.4) | 7.9 | (1.4) | 243.1 | (90) | 29.4 | (7.4) | 0.5 | (0.2) |
| | 1 | 12.2 | (6.8) | 19.4 | (8.9) | 1280.1 | (943.4) | 35.2 | (17) | 1.2 | (0.6) |
| KC3-OA/DOPS | 0.3 | 4.2 | (3.2) | 9.8 | (5.9) | 597.3 | (537.6) | 28.7 | (9.7) | 0.4 | (0.2) |
| | 1 | 10.6 | (9.2) | 20 | (13.6) | 1149.9 | (1071.8) | 36.5 | (16.8) | 0.7 | (0.5) |
| DM2/DPPS | 0.3 | 4.2 | (2.5) | 10.9 | (6.1) | 633.4 | (445.9) | 34.3 | (12.7) | 0.6 | (0.3) |
| | 1 | 7.6 | (1.3) | 16.6 | (2) | 1031.9 | (227.6) | 36.3 | (7.2) | 0.7 | (0.1) |
| unencapsulated | 10 | 0.3 | (0.2) | 1.5 | (0.9) | 21.4 | (6.7) | 30.2 | (7.7) | 0.1 | (0.1) |
| CpG | 50 | 3.9 | (2.1) | 13.5 | (5) | 431.7 | (210.7) | 36.4 | (17.8) | 0.4 | (0.2) |
| | 200 | 5.3 | (0.4) | 16.1 | (3.4) | 5458.7 | (4049.5) | 51.3 | (16.3) | 0.5 | (0.2) |

The draining popliteal, iliac, and inguinal lymph nodes were pooled. Data are the mean (stdev) from 5 mice per group.

TABLE 71
Fold change in mean draining lymph node analyte concentrations
over vehicle 6 hrs post i.m. injection of CpG-based adjuvants.

| | Dose | | | | | | | |
|---|---|---|---|---|---|---|---|---|
| delivery | CpG-ODN | | | | | | | |
| format | (μg) | IFN-g | TNF-a | IL-12p70 | IFN-a | IL-6 | IL-1β | IL-10 |
| vehicle | n/a | 1 | 1 | 1 | 1 | 1 | 1 | 1 |
| KC4-OA/DPPS | 0.3 | 42.3 | 12 | 13 | 5.2 | 66 | 12.8 | 6.5 |
| | 1 | 130.8 | 31 | 75 | 13.7 | 339 | 24 | 6 |
| KC3-OA/DPPS | 0.3 | 39.7 | 10.6 | 10 | 3.4 | 98 | 11.5 | 4.3 |
| | 1 | 113.7 | 30.2 | 76 | 11.7 | 311 | 25.5 | 5.3 |
| KC2-OA/DPPS | 0.3 | 57.8 | 12.6 | 17 | 3.7 | 96 | 14.5 | 3.5 |
| | 1 | 122.3 | 25.6 | 54 | 5.4 | 308 | 26.2 | 4 |
| KC1-OA/DPPS | 0.3 | 44.2 | 8.2 | 9 | 2.2 | 89 | 14.5 | 3.8 |
| | 1 | 155 | 29.4 | 49 | 6.1 | 391 | 37.8 | 4.8 |
| KC3-OA/DOPS | 0.3 | 65.8 | 11.6 | 18 | 5.3 | 136 | 14.5 | 2.8 |
| | 1 | 131 | 24 | 64 | 13.1 | 293 | 20.5 | 3.8 |
| DM2/DPPS | 0.3 | 87.8 | 22.8 | 76 | 14.9 | 158 | 10.5 | 3.8 |
| | 1 | 108 | 31.4 | 122 | 39.3 | 257 | 14.5 | 4.5 |
| unencapsulated | 10 | 1.5 | 2 | 2 | 1.2 | 2 | 3.8 | 0.3 |
| CpG | 50 | 24.8 | 19.4 | 4 | 2.7 | 37 | 20.5 | 5.5 |
| | 200 | 34.5 | 48 | 13 | 4.1 | 109 | 19.2 | 7 |

| | Dose | | | | | |
|---|---|---|---|---|---|---|
| delivery | CpG-ODN | | | | | |
| format | (μg) | KC | MCP-1 | IP-10 | RANTES | GM-CSF |
| vehicle | n/a | 1 | 1 | 1 | 1 | 1 |
| KC4-OA/DPPS | 0.3 | 13.5 | 14.4 | 23.6 | 1.8 | 3 |
| | 1 | 52 | 30.6 | 109.7 | 1.5 | 5 |
| KC3-OA/DPPS | 0.3 | 15 | 12.3 | 22.9 | 1.1 | 2 |
| | 1 | 61 | 29.3 | 108.4 | 1.4 | 4.5 |
| KC2-OA/DPPS | 0.3 | 18 | 14.5 | 44.8 | 1.4 | 3 |
| | 1 | 48.5 | 23 | 79.4 | 1 | 4 |
| KC1-OA/DPPS | 0.3 | 16.5 | 9.9 | 25.1 | 1.2 | 2.5 |
| | 1 | 61 | 24.3 | 132 | 1.5 | 6 |
| KC3-OA/DOPS | 0.3 | 21 | 12.3 | 61.6 | 1.2 | 2 |
| | 1 | 53 | 25 | 118.5 | 1.5 | 3.5 |
| DM2/DPPS | 0.3 | 21 | 13.6 | 65.3 | 1.4 | 3 |
| | 1 | 38 | 20.8 | 106.4 | 1.5 | 3.5 |
| unencapsulated | 10 | 1.5 | 1.9 | 2.2 | 1.3 | 0.5 |
| CpG | 50 | 19.5 | 16.9 | 44.5 | 1.5 | 2 |
| | 200 | 26.5 | 20.1 | 562.8 | 2.1 | 2.5 |

The fold change was not calculated for IFN-β since the vehicle was below the level of detection.

TABLE 72
Concentration (pg/mL) of proinflammatory analytes in blood serum 6 hrs post i.m. injection of UUGU oligoribonucleotide-
based LNP adjuvants using various ICLs. Data are the mean (stdev) from 5 mice per group.

| delivery | Dose UUGU | | | | | | | | | | | | | | |
|---|---|---|---|---|---|---|---|---|---|---|---|---|---|---|---|
| format | oligo (μg) | IFN-g | | TNF-a | | IL-12p70 | | IFN-a | | IL-6 | | IL-1β | IL-10 | | IFN-β |
| KC3-OA/DOPS | 1 | 43.9 | (25.4) | 23.2 | (3.9) | 5.5 | (1.7) | 50.4 | (15.4) | 477.3 | (115.6) | 5.5 (1.8) | 117.8 | (73.9) | 0 (0) |
| KC3-OA | 1 | 11.9 | (4.6) | 24.4 | (5) | 5.3 | (1.5) | 32.1 | (2.7) | 409.3 | (116.4) | 8.7 (7.9) | 181.9 | (130.5) | 0 (0) |
| KC1-OA/DPPS | 1 | 16.3 | (5.5) | 20 | (3.5) | 4.7 | (0.6) | 20 | (3.1) | 295.3 | (119.4) | 6.4 (3.7) | 125.8 | (105.7) | 0 (0) |
| KC1-OA/DOPS | 1 | 16.8 | (9.1) | 19 | (7.2) | 5.1 | (1.5) | 21 | (6.7) | 195 | (128.7) | 3.2 (1.1) | 54.1 | (32.3) | 0 (0) |
| KC1-OA | 1 | 12.4 | (3.2) | 15.2 | (2.6) | 6.6 | (4.1) | 34.8 | (11.8) | 150.7 | (49.3) | 3.8 (1.4) | 80.7 | (104.4) | 0 (0) |
| DM2/DPPS | 1 | 136.4 | (90.4) | 20.7 | (3) | 10.5 | (3.8) | 732.5 | (424.7) | 212 | (63.6) | 9.6 (3.3) | 114.5 | (159.7) | 0 (0) |
| DM2/DOPS | 1 | 234.2 | (103.5) | 27.4 | (5.7) | 9.1 | (2.4) | 485 | (248.7) | 307.1 | (71) | 9.3 (2.6) | 103.6 | (81.2) | 0 (0) |
| DM2 | 1 | 44.8 | (64.5) | 26.3 | (5.4) | 6.1 | (2.3) | 81.5 | (42.8) | 361.6 | (129.7) | 7.3 (3.1) | 41.5 | (36.2) | 0 (0) |
| DODMA/DOPS | 1 | 117.6 | (70) | 19.5 | (3.5) | 6.6 | (3.6) | 57.6 | (20.4) | 381.7 | (114) | 5.8 (1.4) | 63 | (44.3) | 0 (0) |
| DODAP/DPPS | 1 | 9.3 | (4.3) | 13.3 | (3.1) | 2.7 | (0.6) | 18.2 | (2.5) | 22.8 | (28.9) | 8.7 (7.8) | 28.7 | (31.5) | 0 (0) |
| DODAP/DOPS | 1 | 9.8 | (2.9) | 11.2 | (3) | 3.6 | (1) | 24.5 | (11) | 17.9 | (6.6) | 5.3 (3.7) | 83.2 | (80.8) | 0 (0) |
| DODAP | 1 | 9.4 | (3.5) | 16.1 | (2.3) | 4 | (1.2) | 33.1 | (9.1) | 29.6 | (7.3) | 7.9 (7.9) | 69.3 | (53.9) | 0 (0) |
| ALC-0315 | 1 | 15.8 | (12.5) | 25.2 | (9.3) | 3.6 | (0.8) | 49.6 | (19) | 418.3 | (245.6) | 5.7 (2.5) | 73.4 | (97.5) | 0 (0) |
| SM-102 | 1 | 9.7 | (2.1) | 16.8 | (3.3) | 3.6 | (1) | 24.2 | (10.7) | 47.1 | (16.9) | 5.4 (2.7) | 52.3 | (50.8) | 0 (0) |
| DODAP/DOPS | 0.2 | 8.9 | (4.5) | 14.2 | (4) | 3.3 | (1.8) | 18.6 | (6.3) | 16.5 | (5.5) | 2.6 (0.7) | 24 | (31.5) | 0 (0) |
| DM2/DOPS | 0.2 | 18.5 | (11.3) | 11 | (0.7) | 3.1 | (0.9) | 23.8 | (12.3) | 85.1 | (13.9) | 4.4 (1.9) | 86.8 | (109.5) | 0 (0) |
| free UUGU | 100 (free) | 13.9 | (13.1) | 13 | (4.9) | 3.2 | (0.8) | 12.2 | (3.2) | 10.5 | (3.6) | 2.2 (1.9) | 15.5 | (9.4) | 0 (0) |
| (100 ug) | | | | | | | | | | | | | | | |
| vehicle | NA | 11.1 | (2) | 10.4 | (1.6) | 4.2 | (1.7) | 18 | (5.8) | 9.5 | (3) | 1.7 (0.9) | 69 | (77.2) | 0 (0) |

| | Dose | | | | | | | | | | |
|---|---|---|---|---|---|---|---|---|---|---|---|
| delivery | CpG-ODN | | | | | | | | | | |
| format | (μg) | KC | | MCP-1 | | IP-10 | | RANTES | | GM-CSF | |
| KC3-OA/DOPS | 1 | 479.7 | (239.3) | 124.1 | (34.2) | 1769.5 | (262.9) | 28.7 | (11.5) | 4.2 | (2.1) |
| KC3-OA | 1 | 483.6 | (147.1) | 151.8 | (36.6) | 1411.6 | (131.6) | 44.6 | (24.2) | 8.5 | (4.8) |
| KC1-OA/DPPS | 1 | 470.3 | (185.5) | 110.3 | (36.9) | 1370.5 | (231.1) | 22.5 | (6.1) | 3.5 | (1.6) |
| KC1-OA/DOPS | 1 | 362.2 | (157.2) | 65.1 | (19) | 1383.9 | (493.5) | 31.5 | (11) | 6.7 | (5.8) |
| KC1-OA | 1 | 222.5 | (72.6) | 86.8 | (26.8) | 1877.9 | (383.5) | 27.8 | (7.7) | 5.1 | (7.6) |
| DM2/DPPS | 1 | 181.2 | (73.5) | 143.9 | (45.3) | 3752.6 | (812.6) | 31.8 | (6) | 3.5 | (1.7) |
| DM2/DOPS | 1 | 259.9 | (61.3) | 194.1 | (61.8) | 3894.3 | (910.2) | 44.1 | (31.1) | 4.9 | (3) |
| DM2 | 1 | 377.8 | (57.8) | 176.3 | (58) | 2756.3 | (570.8) | 28.9 | (8.2) | 4.9 | (2.8) |
| DODMA/DOPS | 1 | 597.1 | (146.7) | 250.5 | (54.7) | 2549.3 | (506.6) | 41.3 | (14.1) | 8 | (2.8) |
| DODAP/DPPS | 1 | 80.3 | (71.1) | 31 | (8.7) | 1271.2 | (201) | 30.1 | (29.7) | 3.4 | (1.8) |
| DODAP/DOPS | 1 | 87.9 | (38.6) | 34.8 | (11.5) | 1288.3 | (282.3) | 19.7 | (11.4) | 3.2 | (3.5) |
| DODAP | 1 | 124 | (56.2) | 56.1 | (14) | 2093.1 | (297.3) | 72.5 | (77.3) | 1.1 | (1) |
| ALC-0315 | 1 | 420.4 | (179.9) | 136.3 | (69) | 2412.4 | (590.6) | 23.6 | (2.6) | 0.9 | (0.8) |
| SM-102 | 1 | 125.5 | (28.6) | 68.6 | (9) | 2065.5 | (208.5) | 15.5 | (3.4) | 14.5 | (20.8) |
| DODAP/DOPS | 0.2 | 76.1 | (20.4) | 40.9 | (15.5) | 1258.7 | (254.4) | 10.7 | (4) | 4.2 | (4.9) |
| DM2/DOPS | 0.2 | 156.6 | (38.2) | 54.7 | (5.7) | 1207 | (362.2) | 18.1 | (8) | 1.6 | (1.6) |
| free UUGU | 100 (free) | 147 | (140.5) | 36.9 | (10.2) | 799.7 | (83.4) | 21 | (5.9) | 2.2 | (3.7) |
| (100 ug) | | | | | | | | | | | |
| vehicle | NA | 66 | (18.5) | 16.8 | (9.5) | 762.7 | (214.8) | 18.8 | (2.5) | 9.6 | (14.6) |

TABLE 73
Fold change in mean serum analyte concentrations over vehicle
6 hrs post i.m. injection of UUGU-LNP adjuvants.

| delivery | Dose UUGU | | | | | | | |
|---|---|---|---|---|---|---|---|---|
| format | oligo (μg) | IFN-g | TNF-a | IL-12p70 | IFN-a | IL-6 | IL-1β | IL-10 |
| KC3-OA/DOPS | 1 | 4.0 | 2.2 | 1.3 | 2.8 | 50.2 | 3.2 | 1.7 |
| KC3-OA | 1 | 1.1 | 2.3 | 1.3 | 1.8 | 43.1 | 5.1 | 2.6 |
| KC1-OA/DPPS | 1 | 1.5 | 1.9 | 1.1 | 1.1 | 31.1 | 3.8 | 1.8 |
| KC1-OA/DOPS | 1 | 1.5 | 1.8 | 1.2 | 1.2 | 20.5 | 1.9 | 0.8 |
| KC1-OA | 1 | 1.1 | 1.5 | 1.6 | 1.9 | 15.9 | 2.2 | 1.2 |
| DM2/DPPS | 1 | 12.3 | 2.0 | 2.5 | 40.7 | 22.3 | 5.6 | 1.7 |
| DM2/DOPS | 1 | 21.1 | 2.6 | 2.2 | 26.9 | 32.3 | 5.5 | 1.5 |
| DM2 | 1 | 4.0 | 2.5 | 1.5 | 4.5 | 38.1 | 4.3 | 0.6 |
| DODMA/DOPS | 1 | 10.6 | 1.9 | 1.6 | 3.2 | 40.2 | 3.4 | 0.9 |
| DODAP/DPPS | 1 | 0.8 | 1.3 | 0.6 | 1.0 | 2.4 | 5.1 | 0.4 |
| DODAP/DOPS | 1 | 0.9 | 1.1 | 0.9 | 1.4 | 1.9 | 3.1 | 1.2 |
| DODAP | 1 | 0.8 | 1.5 | 1.0 | 1.8 | 3.1 | 4.6 | 1.0 |
| ALC-0315 | 1 | 1.4 | 2.4 | 0.9 | 2.8 | 44.0 | 3.4 | 1.1 |
| SM-102 | 1 | 0.9 | 1.6 | 0.9 | 1.3 | 5.0 | 3.2 | 0.8 |
| DODAP/DOPS | 0.2 | 0.8 | 1.4 | 0.8 | 1.0 | 1.7 | 1.5 | 0.3 |
| DM2/DOPS | 0.2 | 1.7 | 1.1 | 0.7 | 1.3 | 9.0 | 2.6 | 1.3 |
| free UUGU | 100 (free) | 1.3 | 1.3 | 0.8 | 0.7 | 1.1 | 1.3 | 0.2 |
| (100 ug) | | | | | | | | |
| vehicle | NA | 1.0 | 1.0 | 1.0 | 1.0 | 1.0 | 1.0 | 1.0 |

| delivery | Dose UUGU | | | | | |
|---|---|---|---|---|---|---|
| format | oligo (μg) | KC | MCP-1 | IP-10 | RANTES | GM-CSF |
| KC3-OA/DOPS | 1 | 7.3 | 7.4 | 2.3 | 1.5 | 0.4 |
| KC3-OA | 1 | 7.3 | 9.0 | 1.9 | 2.4 | 0.9 |
| KC1-OA/DPPS | 1 | 7.1 | 6.6 | 1.8 | 1.2 | 0.4 |
| KC1-OA/DOPS | 1 | 5.5 | 3.9 | 1.8 | 1.7 | 0.7 |
| KC1-OA | 1 | 3.4 | 5.2 | 2.5 | 1.5 | 0.5 |
| DM2/DPPS | 1 | 2.7 | 8.6 | 4.9 | 1.7 | 0.4 |
| DM2/DOPS | 1 | 3.9 | 11.6 | 5.1 | 2.3 | 0.5 |
| DM2 | 1 | 5.7 | 10.5 | 3.6 | 1.5 | 0.5 |
| DODMA/DOPS | 1 | 9.0 | 14.9 | 3.3 | 2.2 | 0.8 |
| DODAP/DPPS | 1 | 1.2 | 1.8 | 1.7 | 1.6 | 0.4 |
| DODAP/DOPS | 1 | 1.3 | 2.1 | 1.7 | 1.0 | 0.3 |
| DODAP | 1 | 1.9 | 3.3 | 2.7 | 3.9 | 0.1 |
| ALC-0315 | 1 | 6.4 | 8.1 | 3.2 | 1.3 | 0.1 |
| SM-102 | 1 | 1.9 | 4.1 | 2.7 | 0.8 | 1.5 |
| DODAP/DOPS | 0.2 | 1.2 | 2.4 | 1.7 | 0.6 | 0.4 |
| DM2/DOPS | 0.2 | 2.4 | 3.3 | 1.6 | 1.0 | 0.2 |
| free UUGU | 100 (free) | 2.2 | 2.2 | 1.0 | 1.1 | 0.2 |
| (100 ug) | | | | | | |
| vehicle | NA | 1.0 | 1.0 | 1.0 | 1.0 | 1.0 |

The fold change was not calculated for IFN-β because the vehicle was below the level of detection.

TABLE 74
Concentration (pg/mg of tissue) of proinflammatory analytes in draining lymph nodes 6 hrs
post i.m. injection of UUGU oligoribonucleotide-based LNP adjuvants using various ICLs.

| delivery | Dose UUGU | | | | | | | | | | | | | | | |
|---|---|---|---|---|---|---|---|---|---|---|---|---|---|---|---|---|
| format | oligo (ug) | IFN-g | | TNF-a | IL-12p70 | | IFN-a | | IL-6 | | IL-1β | | IL-10 | | IFN-β | |
| KC3-OA/DOPS | 1 | 5.7 | (5.8) | 0.6 (0.3) | 0.1 | (0) | 1.2 | (0.8) | 3.6 | (1.9) | 2.2 | (1.2) | 0.4 | (0.2) | 0.4 | (0.1) |
| KC3-OA | 1 | 0.9 | (0.4) | 0.5 (0.2) | 0.1 | (0) | 1.1 | (0.5) | 4.2 | (3.5) | 2 | (0.8) | 0.4 | (0.1) | 0.4 | (0.1) |
| KC1-OA/DPPS | 1 | 2.9 | (1.9) | 1.1 (0.6) | 0.9 | (1.9) | 1.4 | (1.3) | 6.4 | (2.5) | 6.5 | (2.6) | 0.4 | (0.2) | 0.3 | (0.2) |
| KC1-OA/DOPS | 1 | 1.8 | (2) | 0.6 (0.2) | 0.2 | (0.1) | 0.9 | (0.6) | 3.2 | (3.2) | 3.5 | (1.8) | 0.7 | (0.3) | 0.4 | (0.1) |
| KC1-OA | 1 | 0.4 | (0.2) | 0.5 (0.1) | 0.1 | (0) | 0.8 | (0.5) | 1.3 | (0.9) | 1.3 | (0.5) | 0.6 | (0.1) | 0.4 | (0.1) |
| DM2/DPPS | 1 | 18 | (10.2) | 0.9 (0.3) | 0.4 | (0.4) | 10 | (5.8) | 4.4 | (2.5) | 2.5 | (0.8) | 0.3 | (0.1) | 3.3 | (2.5) |
| DM2/DOPS | 1 | 27.1 | (13.3) | 1.1 (0.3) | 0.1 | (0.1) | 7.7 | (3.7) | 5.6 | (4) | 2.5 | (1.1) | 0.3 | (0.2) | 3.1 | (2.3) |
| DM2 | 1 | 1.4 | (1) | 0.5 (0.3) | 0.1 | (0) | 2.2 | (1.9) | 3.6 | (0.8) | 1.6 | (0.4) | 0.3 | (0.1) | 0.5 | (0.5) |
| DODMA/DOPS | 1 | 19.9 | (19.6) | 0.9 (0.5) | 0.3 | (0.5) | 3 | (2) | 8.7 | (6.8) | 3.3 | (1.6) | 0.2 | (0.1) | 0.3 | (0.1) |
| DODAP/DPPS | 1 | 0.4 | (0.1) | 0.3 (0.1) | 0.1 | (0) | 0.6 | (0.3) | 0.2 | (0.1) | 1.3 | (0.4) | 0.3 | (0) | 0.1 | (0.1) |
| DODAP/DOPS | 1 | 0.3 | (0.2) | 0.3 (0.2) | 0.1 | (0) | 0.6 | (0.5) | 0.3 | (0.3) | 1.1 | (0.7) | 0.2 | (0.1) | 0.1 | (0.1) |
| DODAP | 1 | 0.3 | (0.2) | 0.3 (0.2) | 0.1 | (0.1) | 1.1 | (0.7) | 0.6 | (0.3) | 0.9 | (0.2) | 0.2 | (0.1) | 0.1 | (0.1) |
| ALC-0315 | 1 | 0.3 | (0.2) | 0.3 (0.2) | 0.1 | (0) | 1.2 | (1.1) | 4.2 | (3.8) | 1.5 | (0.4) | 0.2 | (0.1) | 0.1 | (0.1) |
| SM-102 | 1 | 0.8 | (0.8) | 0.6 (0.8) | 0.1 | (0.1) | 1.2 | (1.4) | 2.3 | (4) | 2.2 | (2) | 0.5 | (0.4) | 0.2 | (0.2) |
| DODAP/DOPS | 0.2 | 1.7 | (3) | 0.3 (0.1) | 0.1 | (0) | 0.6 | (0.4) | 0.5 | (0.3) | 0.9 | (0.2) | 0.4 | (0.2) | 0.2 | (0.1) |
| DM2/DOPS | 0.2 | 1 | (0.7) | 0.3 (0.1) | 0.1 | (0) | 0.6 | (0.2) | 0.7 | (0.4) | 1.1 | (0.5) | 0.2 | (0) | 0.1 | (0) |
| free UUGU | 100 (free) | 0.2 | (0.1) | 0.3 (0.1) | 0.1 | (0.1) | 0.5 | (0.2) | 0.6 | (0.2) | 0.6 | (0.3) | 0.4 | (0.1) | 0.4 | (0.3) |
| (100 ug) | | | | | | | | | | | | | | | | |
| vehicle | NA | 0.1 | (0.1) | 0.1 (0.1) | 0.1 | (0.1) | 3.4 | (4.6) | 0.7 | (1.1) | 1 | (0.7) | 0.3 | (0.2) | 0.2 | (0.2) |

| delivery | Dose UUGU | | | | | | | | | | |
|---|---|---|---|---|---|---|---|---|---|---|---|
| format | oligo (ug) | KC | | MCP-1 | | IP-10 | | RANTES | | GM-CSF | |
| KC3-OA/DOPS | 1 | 1.4 | (0.5) | 2.7 | (1.5) | 114.6 | (53.3) | 25.8 | (6.2) | 0.2 | (0.1) |
| KC3-OA | 1 | 2.5 | (1.9) | 3.2 | (1.8) | 106 | (58.6) | 26.5 | (7.3) | 0.2 | (0) |
| KC1-OA/DPPS | 1 | 2.7 | (1) | 2.8 | (1.1) | 145.4 | (70.2) | 35.6 | (19.6) | 0.3 | (0.2) |
| KC1-OA/DOPS | 1 | 1.4 | (0.7) | 1.4 | (0.6) | 67.8 | (43.2) | 34.5 | (11.7) | 0.4 | (0) |
| KC1-OA | 1 | 0.7 | (0.3) | 1.4 | (0.7) | 69.1 | (37.2) | 43.1 | (11) | 0.3 | (0.1) |
| DM2/DPPS | 1 | 0.8 | (0.5) | 2.8 | (1.6) | 276.8 | (136.7) | 30 | (9.6) | 0.3 | (0) |
| DM2/DOPS | 1 | 1.2 | (0.7) | 5.5 | (2.5) | 563.6 | (442.8) | 35.8 | (8.8) | 0.2 | (0.1) |
| DM2 | 1 | 1.3 | (0.3) | 2.6 | (1) | 168.3 | (63.5) | 35.2 | (11.1) | 0.2 | (0) |
| DODMA/DOPS | 1 | 2.8 | (1.6) | 7.2 | (4.5) | 235.8 | (160.9) | 29.5 | (6.4) | 0.3 | (0.1) |
| DODAP/DPPS | 1 | 0.3 | (0.1) | 0.4 | (0.2) | 74.8 | (21.2) | 24.3 | (4.2) | 0.2 | (0) |
| DODAP/DOPS | 1 | 0.2 | (0.2) | 0.4 | (0.4) | 52.6 | (33.1) | 17.2 | (10.5) | 0.1 | (0.1) |
| DODAP | 1 | 0.2 | (0.1) | 0.6 | (0.7) | 60.7 | (31) | 25 | (5.7) | 0.1 | (0.1) |
| ALC-0315 | 1 | 1.2 | (0.9) | 1.4 | (1.1) | 127.3 | (37.9) | 22.6 | (2.5) | 0.1 | (0.1) |
| SM-102 | 1 | 0.7 | (0.9) | 2.8 | (4.3) | 168.6 | (185.3) | 50.2 | (52.5) | 0.3 | (0.2) |
| DODAP/DOPS | 0.2 | 0.3 | (0.1) | 0.7 | (0.4) | 53.4 | (36) | 27.8 | (5.6) | 0.2 | (0.1) |
| DM2/DOPS | 0.2 | 0.4 | (0.2) | 0.7 | (0.3) | 73 | (32.7) | 27.3 | (5.2) | 0.2 | (0.1) |
| free UUGU | 100 (free) | 0.2 | (0.1) | 0.4 | (0.2) | 7.7 | (5) | 19.7 | (13.2) | 0.3 | (0.1) |
| (100 ug) | | | | | | | | | | | |
| vehicle | NA | 0.1 | (0.1) | 0.2 | (0.3) | 12.6 | (8.5) | 20.6 | (11.7) | 0.1 | (0.1) |

The draining popliteal, iliac, and inguinal lymph nodes were pooled. Data are the mean (stdev) from 5 mice per group.

TABLE 75
Fold change in mean draining lymph node analyte concentrations over vehicle 6 hrs post
i.m. injection of UUGU oligoribonucleotide-based LNP adjuvants using various ICLs.

| delivery | Dose UUGU | | | | | | | | |
|---|---|---|---|---|---|---|---|---|---|
| format | oligo (μg) | IFN-g | TNF-a | IL-12p70 | IFN-a | IL-6 | IL-1β | IL-10 | IFN-β |
| KC3-OA/DOPS | 1 | 57 | 6 | 1 | 0.4 | 5.1 | 2.2 | 1.3 | 2.0 |
| KC3-OA | 1 | 9 | 5 | 1 | 0.3 | 6.0 | 2 | 1.3 | 2.0 |
| KC1-OA/DPPS | 1 | 29 | 11 | 9 | 0.4 | 9.1 | 6.5 | 1.3 | 1.5 |
| KC1-OA/DOPS | 1 | 18 | 6 | 2 | 0.3 | 4.6 | 3.5 | 2.3 | 2.0 |
| KC1-OA | 1 | 4 | 5 | 1 | 0.2 | 1.9 | 1.3 | 2.0 | 2.0 |
| DM2/DPPS | 1 | 180 | 9 | 4 | 2.9 | 6.3 | 2.5 | 1.0 | 16.5 |
| DM2/DOPS | 1 | 271 | 11 | 1 | 2.3 | 8.0 | 2.5 | 1.0 | 15.5 |
| DM2 | 1 | 14 | 5 | 1 | 0.6 | 5.1 | 1.6 | 1.0 | 2.5 |
| DODMA/DOPS | 1 | 199 | 9 | 3 | 0.9 | 12.4 | 3.3 | 0.7 | 1.5 |
| DODAP/DPPS | 1 | 4 | 3 | 1 | 0.2 | 0.3 | 1.3 | 1.0 | 0.5 |
| DODAP/DOPS | 1 | 3 | 3 | 1 | 0.2 | 0.4 | 1.1 | 0.7 | 0.5 |
| DODAP | 1 | 3 | 3 | 1 | 0.3 | 0.9 | 0.9 | 0.7 | 0.5 |
| ALC-0315 | 1 | 3 | 3 | 1 | 0.4 | 6.0 | 1.5 | 0.7 | 0.5 |
| SM-102 | 1 | 8 | 6 | 1 | 0.4 | 3.3 | 2.2 | 1.7 | 1.0 |
| DODAP/DOPS | 0.2 | 17 | 3 | 1 | 0.2 | 0.7 | 0.9 | 1.3 | 1.0 |
| DM2/DOPS | 0.2 | 10 | 3 | 1 | 0.2 | 1.0 | 1.1 | 0.7 | 0.5 |
| free UUGU | 100 (free) | 2 | 3 | 1 | 0.1 | 0.9 | 0.6 | 1.3 | 2.0 |
| (100 ug) | | | | | | | | | |
| vehicle | NA | 1 | 1 | 1 | 1.0 | 1.0 | 1 | 1 | 1 |

| delivery | Dose UUGU | | | | | |
|---|---|---|---|---|---|---|
| format | oligo (μg) | KC | MCP-1 | IP-10 | RANTES | GM-CSF |
| KC3-OA/DOPS | 1 | 14.0 | 13.5 | 9.1 | 1.3 | 2.0 |
| KC3-OA | 1 | 25.0 | 16.0 | 8.4 | 1.3 | 2.0 |
| KC1-OA/DPPS | 1 | 27.0 | 14.0 | 11.5 | 1.7 | 3.0 |
| KC1-OA/DOPS | 1 | 14.0 | 7.0 | 5.4 | 1.7 | 4.0 |
| KC1-OA | 1 | 7.0 | 7.0 | 5.5 | 2.1 | 3.0 |
| DM2/DPPS | 1 | 8.0 | 14.0 | 22.0 | 1.5 | 3.0 |
| DM2/DOPS | 1 | 12.0 | 27.5 | 44.7 | 1.7 | 2.0 |
| DM2 | 1 | 13.0 | 13.0 | 13.4 | 1.7 | 2.0 |
| DODMA/DOPS | 1 | 28.0 | 36.0 | 18.7 | 1.4 | 3.0 |
| DODAP/DPPS | 1 | 3.0 | 2.0 | 5.9 | 1.2 | 2.0 |
| DODAP/DOPS | 1 | 2.0 | 2.0 | 4.2 | 0.8 | 1.0 |
| DODAP | 1 | 2.0 | 3.0 | 4.8 | 1.2 | 1.0 |
| ALC-0315 | 1 | 12.0 | 7.0 | 10.1 | 1.1 | 1.0 |
| SM-102 | 1 | 7.0 | 14.0 | 13.4 | 2.4 | 3.0 |
| DODAP/DOPS | 0.2 | 3.0 | 3.5 | 4.2 | 1.3 | 2.0 |
| DM2/DOPS | 0.2 | 4.0 | 3.5 | 5.8 | 1.3 | 2.0 |
| free UUGU | 100 (free) | 2.0 | 2.0 | 0.6 | 1.0 | 3.0 |
| (100 ug) | | | | | | |
| vehicle | NA | 1.0 | 1.0 | 1.0 | 1.0 | 1.0 |

### Example 29. Preparing a TLR7/8 Agonist 1-PEG-Lipid Conjugate and Incorporation into LNP

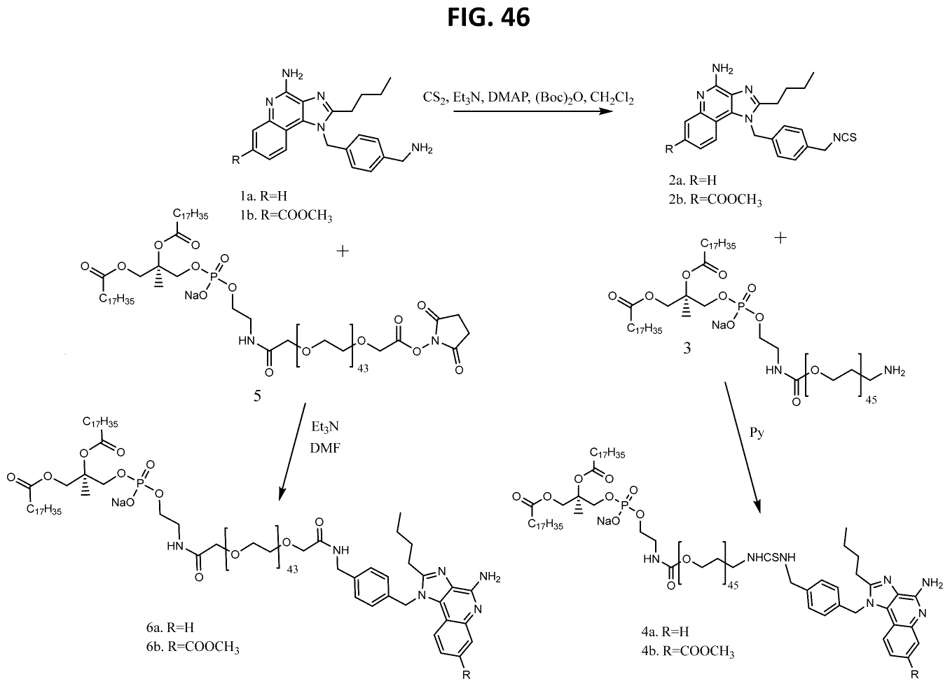

The synthetic scheme is represented on FIG. 46. TLR7/8 Agonist 1 (Cat. No HY-103698, MedChemExpress, New Jersey, USA) (1a) is converted into thiocyanate 2a as described in Shukla et al., 2010, Bioorg Med Chem Lett. 20:6384-6386 (Compound 5d). Two mol parts of isothiocyanate 2a are treated with one mol part of DSPE-PEG(2000)-amine (1,2-distearoyl-sn-glycero-3-phosphoethanolamine-N-(amino(polyethylene glycol)-2000) NH₄ salt, Cat. No. 880132, Avanti Polar Lipids, Alabama, USA) (3) in pyridine at 45° C. for 18 hours as described in Shukla, et al, 2010, above. After removal of pyridine under vacuum, the residue is purified by column chromatography in methanol-chloroform to obtain conjugate 4a.

Alternatively, TLR7/8 Agonist 1 (1a) is treated with 1 mol part DSPE-PEG(2000)-carboxyNHS (1,2-distearoyl-sn-glycero-3-phosphoethanolamine-N-(carboxy(polyethylene glycol)-2000, NHS ester, Na salt, Cat. No. 880138, Avanti Polar Lipids, Alabama, USA) (5) in the presence of triethylamine in DMF at 30° C. overnight. The conjugate (6a) is purified by column chromatography.

The prepared conjugate is dissolved in ethanol, added to the ethanolic solution of the LNP lipids, and the LNP are prepared as in Example 2B, using conjugate 4a (or 6a), KC3-OA ionizable cationic lipid, DSPC, DPPS, Cholesterol and PEG-DMG in relative molar amounts X/48/5/5/(40.5−X)/1.5, where X varies from 0.1 to 2, and the final formulation buffer is 10% sucrose, 10 mM Tris-HCl pH 7.4. The amount of the conjugate is determined by HPLC after solubilization of the LNPs in isopropanol. LNPs are tested for antibody production and T-cell response in mice essentially as described in Example 4 above.

Alternatively, the conjugate is dissolved in the aqueous buffer containing mRNA, and the LNPs are formed by combining the solution with the ethanolic solution of the lipids KC3-OA, DSPC, DPPS, Cholesterol, and PEG-DMG (48/5/5/40.5/1.5 molar parts) in a microfluidic Y-junction mixing device (Example 2B). The LNPs are purified from the free (unencapsulated) conjugate by buffer exchange using Amicon centrifugal concentrators. The purified LNPs are tested for antibody production and T-cell response in mice as described in Example 4.

### Example 30. Preparing a TLR7/8 Agonist-6-PEG-Lipid Conjugate and Incorporation into LNP

The protocols of Example 29 are applied to TLR7/8 Agonist 6 (Cat. No. HY-145885. MedChem Express, New Jersey, USA, 1b) (Yang et al. 2022, Bioorg Med Chem Lett. 59:128548) to prepare conjugates 4b and 6b (FIG. 46), incorporate into LNP, and test the LNP for the antibody production and T-cell response in mice.

### Example 31. Preparation and Analysis of LNPs Containing OA-DM2

The purpose of this study was to compare the following LNP formulated TLR adjuvants for activity in vivo. OA-DM2 based LNPs containing either the TLR 9 agonist 1018 CpG or the TLRs 7/8 agonist UUGU were prepared and characterized as described in Examples above including Example 16. A LNP formulation of each oligo was prepared with 48 mol % OA-DM2, mol % DSPC, 5 mol % DOPS, 38.5 mol % cholesterol, and 1.5 mol % PEG-DMG with an N/P of 5.25 or 48 mol % OA-DM2, 10 mol % DSPC, 38.5 mol % cholesterol, and 1.5 mol % PEG-DMG with an N/P of 5.25

Particle size refers to the Z-Average particle size. Post-F/T refers to samples that were measured after a freeze/thaw event, and Zeta refers to zeta potential measurements at either pH 5 or pH 7 in units of mV. Oligonucleotide encapsulation efficiency is referred to as (E.E,%).

TABLE 76
Physicochemical properties and characterization of agonist containing
LNPs. In the column on the left, for the appropriate formulations,
the presence of the anionic lipid DOPS is indicated.

| | | Particle | Particle | | | |
|---|---|---|---|---|---|---|
| Lipid | | Size | Size (nm) | E.E, %, | Zeta | Zeta |
| Composition | Oligo | (nm) | Post-F/T | stdev | pH 5 | pH 7 |
| OA-DM2 | CpG | 103.6 | 102.5 | 82.76 ± 1.37 | 28.49 | 10.90 |
| | 1018 | | | | | |
| OA-DM2/ | CpG | 116.2 | 119.9 | 86.69 ± 1.98 | 27.79 | 8.14 |
| DOPS | 1018 | | | | | |
| OA-DM2 | UUGU | 94.46 | 93.22 | 88.36 ± 2.95 | 29.43 | 10.50 |
| OA-DM2/ | UUGU | 101.6 | 101.9 | 91.25 ± 1.92 | 25.84 | 6.48 |
| DOPS | | | | | | |

The formulations ranged in size from approximately 93 to 120 nm in diameter where the formulations containing DOPS were larger than those without DOPS and all formulations had less than 3.2% change in particle size after freezing and thawing. The % entrapped oligo ranged from 83-91% and LNPs exhibited positive surface charge at pH 5 (+25 to +29 mV) and less positively charged at pH 7 (+6.5 to 10.9 mV).

### Example 32. Varicella Zoster Virus gE Subunit Vaccination and the Adjuvant Activity of TLR9 Agonist CpG-LNPs, TLR7/8 UUGU-LNPs, and the Commercially Available Adjuvant AS01

The primary purpose of adjuvants is to improve adaptive immune responses against vaccine antigens. The shingles vaccine is an approved vaccine, trade name Shingrix (GlaxoSmithKline), to prevent reactivation of the varicella zoster virus (VZV) and the development of herpes zoster, also referred to as shingles. The FDA-approved vaccine consists of recombinant VZV surface glycoprotein E (gE) and ASO1_{B} adjuvant. The purpose of this study was to examine a dose range of the TLR agonist CpG-1018 ODN (SEQ ID NO: 2) or TLR7/8 ssRNA oligonucleotide UUGU (SEQ ID NO: 25) encapsulated in LNPs. The adjuvant activity of these LNP adjuvants was compared to AS01, which was obtained from purchased Shingrix vaccine.

OA-DM2-based LNPs containing either CpG-1018 or UUGU were prepared and characterized as described in Examples 19 and 20. A PS-targeted LNP formulation of each oligo was prepared with 48 mol % OA-DM2, 5 mol % DSPC, 5 mol % DOPS, 38.5 mol % cholesterol, and 1.5 mol % PEG-DMG with an N/P of 5.25. Untargeted LNPs were prepared with 48 mol % OA-DM2, 10 mol % DSPC, 38.5 mol % cholesterol, and 1.5 mol % PEG-DMG with an N/P of 5.25. Particle size refers to the Z-Average particle size. Post-F/T refers to samples that were measured after a freeze/thaw event, and Zeta refers to zeta potential measurements at either pH 5 or pH 7 in units of mV. Oligonucleotide encapsulation efficiency is referred to as (E.E,%). The formulations ranged in size from approximately 93 to 120 nm in diameter where the formulations containing DOPS were larger than those without DOPS and all formulations had less than 3.2% change in particle size after freezing and thawing. The % entrapped oligo ranged from 83-91% and LNPs exhibited positive surface charge at pH 5 (˜+25 to +29 mV) and less positively charged at pH 7 (+6.5 to 10.9 mV).

TABLE 77
Physicochemical properties and characterization of TLR agonist-
containing LNPs. In the column on the left, for the appropriate
formulations, the presence of the anionic lipid DOPS is indicated.

| | | Particle | Particle | | | |
|---|---|---|---|---|---|---|
| Lipid | | Size | Size (nm) | E.E, %, | Zeta | Zeta |
| Composition | Oligo | (nm) | Post-F/T | stdev | pH 5 | pH 7 |
| OA-DM2 | CpG | 103.6 | 102.5 | 82.76 ± 1.37 | 28.49 | 10.90 |
| | 1018 | | | | | |
| OA-DM2/ | CpG | 116.2 | 119.9 | 86.69 ± 1.98 | 27.79 | 8.14 |
| DOPS | 1018 | | | | | |
| OA-DM2 | UUGU | 94.46 | 93.22 | 88.36 ± 2.95 | 29.43 | 10.50 |
| OA-DM2/ | UUGU | 101.6 | 101.9 | 91.25 ± 1.92 | 25.84 | 6.48 |
| DOPS | | | | | | |

All CB6F1 mice were injected i.m. with 2 ug gE sourced from a purchased vial of Shingrix vaccine. The protein sequence for gE is shown in SEQ ID NO: 171. Adjuvant was co-injected with resuspended gE protein. Specifically, due to the presence of Tween 80 (polysorbate 80) in the gE lyophilization product, which breaks down LNPs, adjuvant was injected first immediately followed by the gE antigen in the same site. Mice were given a homologous boost 24 days later, and 12 days later spleens were collected and splenocytes were stimulated with a gE overlapping peptide pool to identify cytokine-producing T cells by flow, defined as T cells that produced IFN-g or IL-2. Nonparametric one-way ANOVA was used with multiple post-tests to compare all groups with the adjuvant alone negative control group.

Mice that received adjuvant alone did not have a detectable T cell response (FIG. 47A-47C). Three AS01 adjuvant dose levels were used: 2, 1 and 0.5 ug. Only the 2 ug dose induced a CD4 response above background. No CD8 T cell responses were observed, indicating that gE likely does not contain a good MHC class I epitope. Soluble antigens are also known to be poor inducers of CD8 T cell responses. Of the groups that received CpG-1018 in various formats, the OA-DM2/DOPS LNPs at 1.5 and 0.5 ug and OA-DM2 LNPs at the 1.5 ug dose generated robust CD4 T cell responses (FIG. 47A). Two out of five mice in the 1.5 ug OA-DM2/DOPS group had CD4 responses >1% of CD4 T cells. FIG. 47B shows the results from the UUGU adjuvant groups in comparison with AS01. Only the 1.5 ug UUGU OA/DM2/DOPS adjuvant induced a CD4 response above background. Unencapsulated free CpG-1018 or UUGU did not induce CD4 response above the non-adjuvant group, demonstrating that this format is highly inefficient, which is well known in the vaccine field. Of the adjuvant groups that had detectable gE-specific CD4 T cell responses, there was a difference in the cytokine profiles: relative to AS01, a greater proportion of CD4 T cells activated in the presence of CpG OA-DM2/DOPS were IFN-g single-producers (FIG. 47C). AS01 and UUGU OA-DM2/DOPS adjuvants tended to create more IL-2 single-producing CD4s.

In conclusion, OA-DM2 LNP delivery of CpG is greater than 100-fold more efficient (compare the 0.5 ug OA-DM2/DOPS dose to the free 50 ug CpG) and LNP delivery of UUGU is at least 25-fold more efficient than free UUGU (compare 1.5 ug OA/DM2/DOPS to 40 ug free UUGU). Although not statistically significant, DOPS targeting of CpG or UUGU trended toward superior activity. These formulations were as efficient as 2 ug AS01, which is known as a potent adjuvant used in multiple approved vaccines.

Human varicella zoster virus gE antigen.
SEQ ID NO: 171
MGTVNKPVVGVLMGFGIITGTLRITNPVRASVLRYDDFHIDEDKL
DTNSVYEPYYHSDHAESSWVNRGESSRKAYDHNSPYIWPRNDYDG
FLENAHEHHGVYNQGRGIDSGERLMQPTQMSAQEDLGDDTGIHVI
PTLNGDDRHKIVNVDQRQYGDVFKGDLNPKPQGQRLIEVSVEENH
PFTLRAPIQRIYGVRYTETWSFLPSLTCTGDAAPAIQHICLKHTT
CFQDVVVDVDCAENTKEDQLAEISYRFQGKKEADQPWIVVNTSTL
FDELELDPPEIEPGVLKVLRTEKQYLGVYIWNMRGSDGTSTYATF
LVTWKGDEKTRNPTPAVTPQPRGAEFHMWNYHSHVFSVGDTFSLA
MHLQYKIHEAPFDLLLEWLYVPIDPTCQPMRLYSTCLYHPNAPQC
LSHMNSGCTFTSPHLAQRVASTVYQNCEHADNYTAYCLGISHMEP
SFGLILHDGGTTLKFVDTPESLSGLYVFVVYFNGHVEAVAYTVVS
TVDHFVNAIEERGFPPTAGQPPATTKPKEITPVNPGTSPLLRYAA
WTGGLAAVVLLCLVIFLICTAKRMRVKAYRVDKSPYNQSMYYAGL
PVDDFEDSESTDTEEEFGNAIGGSHGGSSYTVYIDKTR.

### Example 33. CpG/UUGU-LNP Adjuvants Induce Robust B Cell Responses and Antibody Titers

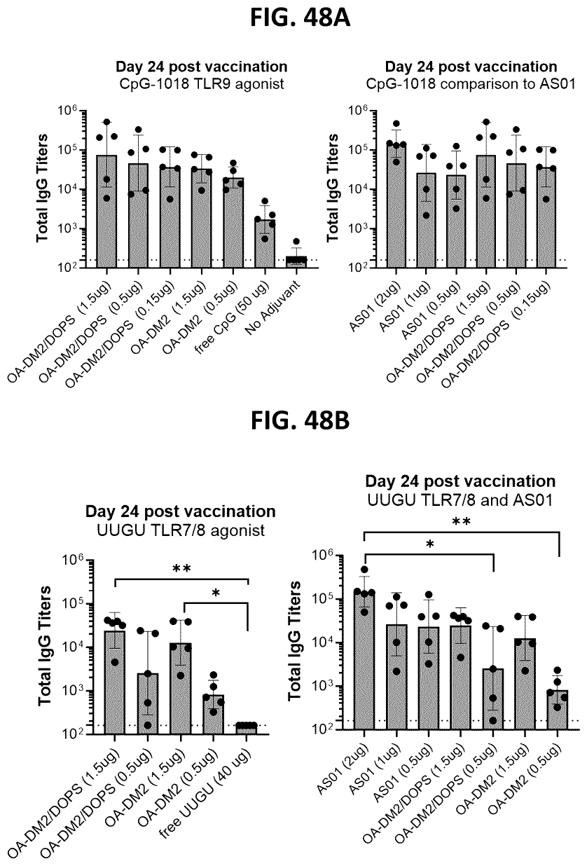

Within the same experiment as Example 31, total anti-gE antibody titers were quantified by standard limiting-dilution ELISA. As with the T cell response, both the UUGU and CpG oligos encapsulated in OA-DM2 LNPs were effective at generating IgG titers. Total binding IgG titers 24 days after the first immunization are shown in FIG. 48A-48B. In the absence of adjuvant, no titers were observed at a dilution >1:200, which was the limit of detection used in the ELISAs. CpG OA-DM2 was similarly effective at 1.5, 0.5 and 0.15 ug (FIG. 48A, left plot), and induced ˜10-fold high serum antibody levels than 50 ug free CpG. The CpG-LNP adjuvants were also as effective as AS01 at the 2 ug dose (FIG. 48A, right plot). There was no difference between DOPS or untargeted CpG-LNPs.

For UUGU adjuvant, there was a decline in IgG Ab titers from the 1.5 ug to 0.5 ug dose, and there was no difference between DOPS-targeted and untargeted LNP formulations (FIG. 48B, left panel). Free UUGU was an inefficient agonist, as no titers were observed at a dilution >1:200, which was the limit of detection used in the ELISAs. The 1.5 ug UUGU-LNP adjuvants were as effective as AS01 at all dose levels (FIG. 48B, right panel).

In conclusion, free CpG-1018 or ssRNA UUGU oligos are highly inefficient TLR agonists, and this is despite having phosphorothioate linkages to improve resistance to nuclease degradation. This is likely a result of extracellular degradation by nucleases, poor uptake by TLR7/8/9-expressing innate immune cells, and rapid dissemination out of the draining LN. By encapsulating these in an LNP, these limitations are overcome and their potency as adjuvants is vastly improved.

### Example 34. Blood Inflammatory Profiles of CpG-LNP and UUGU-LNP Adjuvants

Within the same study in Examples 31 and 32, blood was collected 6 hours after i.m. vaccination and acute pro-inflammatory analyte concentrations were measured in the blood using the LEGENDplex platform. We demonstrated in Example 21 and Example 22 that UUGU-LNPs and CpG-LNPs, respectively, induce acute production of cytokines and chemokines downstream from TLR7/8 and TLR9 signaling pathways in the draining lymph node and blood. The induction of pro-inflammatory analytes for both UUGU- and CpG-LNPs was more potent with PS-targeted LNPs compared to non-targeted LNPs. See Example 27 for a comparison of targeted vs. untargeted CpG-LNPs and Example 28 for UUGU-LNPs.

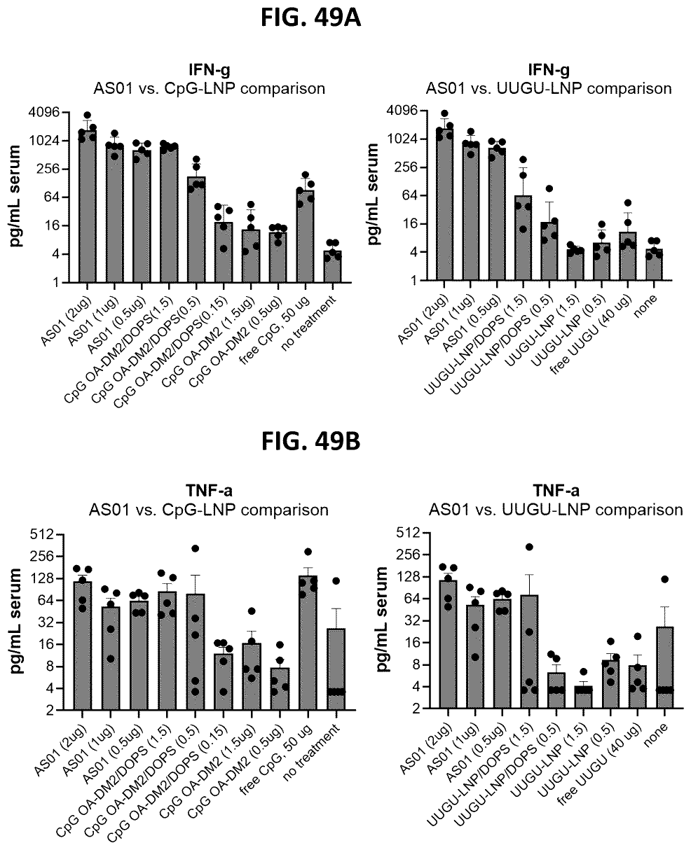

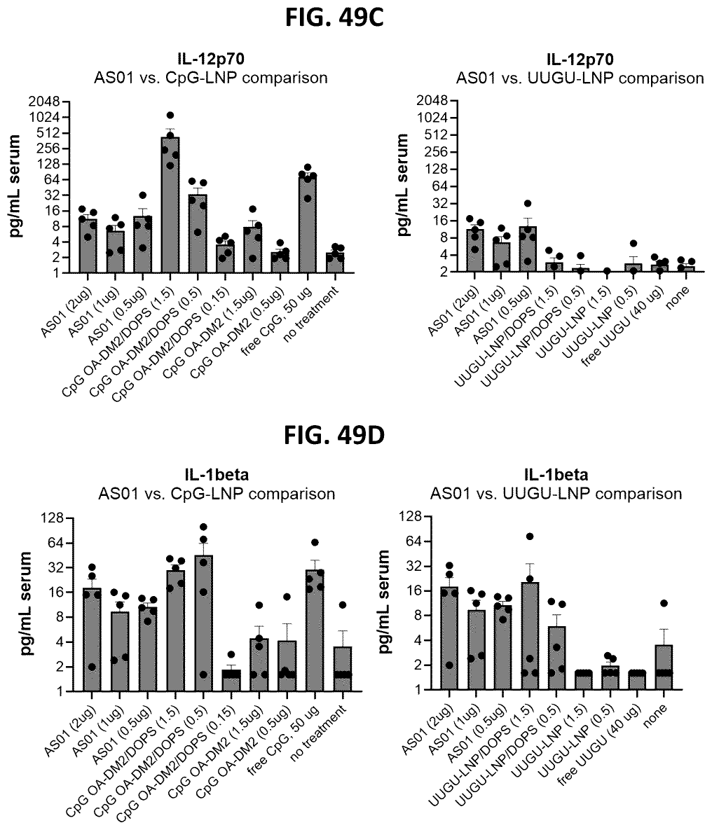

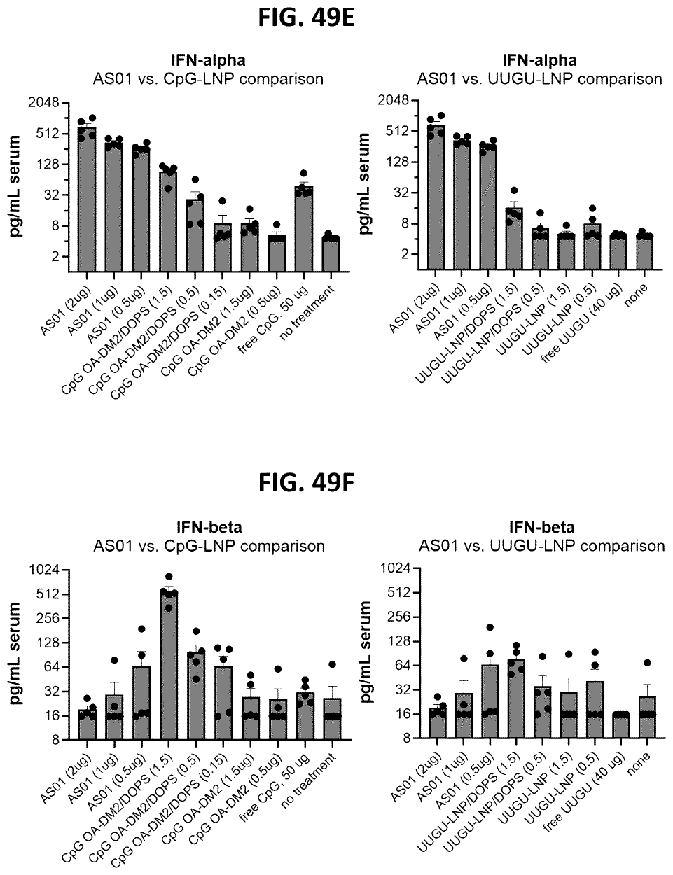

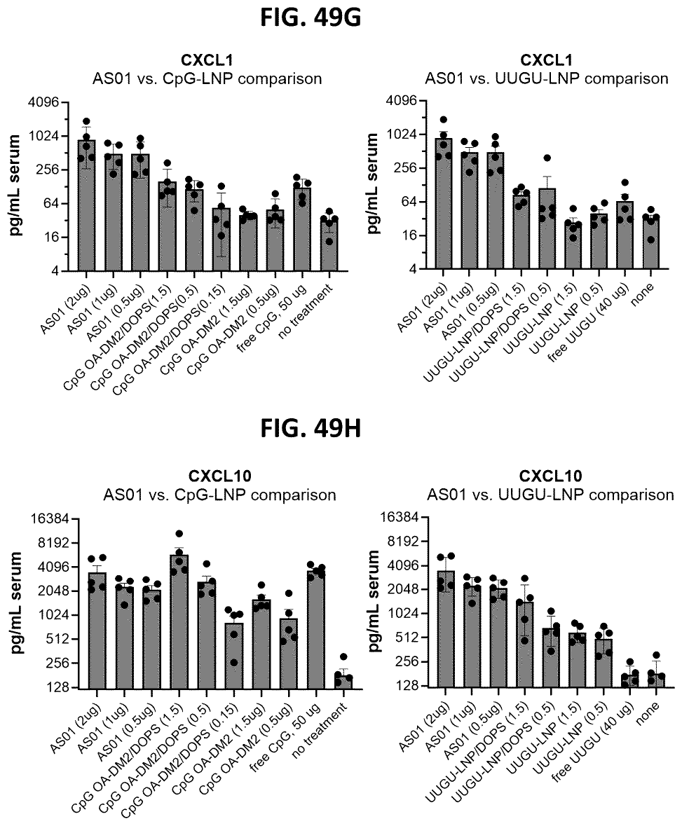

When co-administered with gE antigen, the previously observed difference in potency was again apparent between PS-targeted (DOPS) and untargeted formulations (FIG. 49A-49H). There were similar cytokine patterns between AS01 and CpG OA-DM2/DOPS of IFN-g (FIG. 49A), TNF-α (FIG. 49B), IL-1beta (FIG. 49D), CXCL1 (FIG. 49G) and CXCL10 (FIG. 49H). Several notable differences between AS01 and CpG OA-DM2/DOPS were observed: the induction of IL-12 by CpG but not by AS01 (FIG. 49C), lower levels of IFN-alpha were induced by CpG compared to AS01 (FIG. 49E), IFN-beta was induced by CpG whereas levels were minimal for AS01 (FIG. 49F).

The blood analyte concentrations in UUGU-LNP-treated mice were distinct from AS01 and CpG-LNP. UUGU-LNPs induced minimal production of all of these cytokines and chemokines with the exception of CXCL10. This is consistent with other studies where we examined the inflammatory profile of UUGU-LNPs in the absence of any antigen. Given the robust levels of anti-gE IgG in these same animals (FIG. 48A-48B), UUGU-LNPs are clearly active and are able to stimulate the innate immune system. We may be capturing the acute response after UUGU-LNP injection due to a difference in kinetics (we only sampled at 6 hrs, when we know levels are high after CpG-LNP treatment), and the panel of analytes may have been too limited.

### Example 35. Anti-HBsAg Antibody Titers after Immunization with CpG- or UUGU-LNPs

Hepatitis B virus (HBV) infects the liver and can cause cirrhosis and hepatocellular carcinoma. The WHO estimates that approximately 250 million people are chronically infected despite the approval of multiple effective HBV vaccines. The major vaccine antigen is the HBV surface antigen (HBsAg) and early HBV vaccines consisted of HBsAg absorbed to alum (e.g. PREHEVBRIO and ENGERIX-B), which acts as an adjuvant and antigen depot. A protein sequence for HBsAg is shown in SEQ ID NO: 178. More recently, a new and more effective HBV vaccine, trade name HEPLISAV-B, was approved that uses CpG-1018. A standard vaccine dose in humans consists of 20 ug HBsAg and 3000 ug CpG-1018 ODN. The high adjuvant to antigen ratio is a testament to how inefficiently naked CpG-1018 is taken up by TLR9-expressing immune cells, which in humans is primarily limited to B cells and plasmacytoid DCs. The purpose of this study was to examine how well CpG 1018- or UUGU-LNPs boost antibody titers to HBsAg. CB6F1 mice were immunized with 1 ug HBsAg in combination with various formulations of CpG and UUGU oligos. Serum was collected on day 24 and analyzed by ELISA for total binding IgG titers.

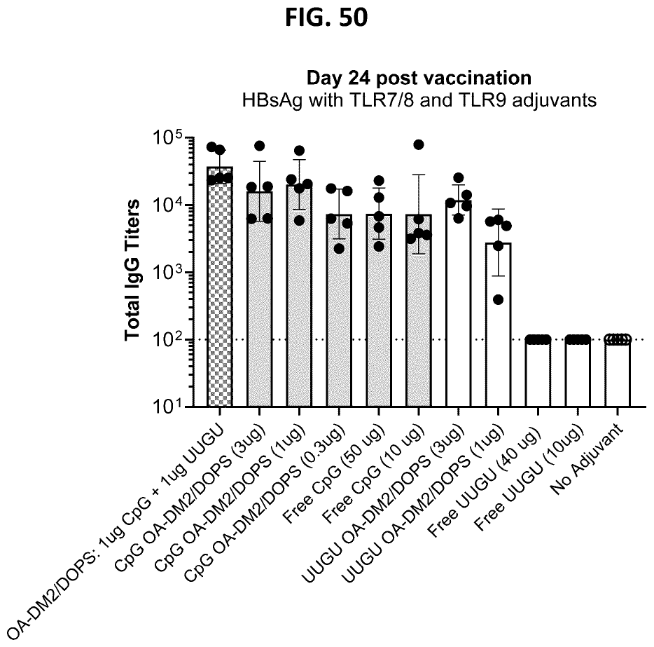

In the absence of any adjuvant, anti-HBsAg titers were below a titer of 1:200, which was the lower limit of detection for this ELISA (FIG. 50). The same was true for 40 μg and 10 μg of free UUGU oligo. In contrast, both 50 and 10 ug free CpG induced titers. Equivalent titers were generated when CpG OA-DM2/DOPS adjuvant was used at 3, 1 or 0.3 ug or UUGU OA-DM2/DOPS adjuvant was used at 3 or 1 ug. In one group, 1 ug CpG OA-DM2/DOPS and 1 ug UUGU OA-DM2/DOPS was co-administered with HbsAg to test for synergy. The geometric mean titer for this group was the highest of all groups, and although this was not statistically significant, the spread in titers between animals was less compared to the other groups.

In conclusion, CpG-1018 and UUGU oligos formulated with OA-DM2/DOPS LNPs were highly active as an adjuvant for HBsAg and could be a replacement for free CpG and further increase vaccine efficacy.

HBsAg. S antigen.
SEQ ID NO: 178
MENITSGFLGPLLVLQAGFFLLTRILTIPQSLDSWWTSLNFLGGS
PVCLGQNSQSPTSNHSPTSCPPICPGYRWMCLRRFIIFLFILLLC
LIFLL VLLDYQGMLPVCPLIPGSTTTNTGPCKTCTTPAQGNSMF
PSCCCTKPTDGNCTCIPIPSSWAFAKYLWEWASVRFSWLSLLVPF
VQWFVGLSPTVWLSAIWMMWYWGPSLYSIVSPFIPLLPIFFCLWV
YI.

### Example 36. Anti-RSVpreF Antibody Titers After Immunization with CpG-LNPs

Respiratory syncytial virus (RSV) is the most common cause of hospitalization of infants and children <5 years of age for lower respiratory disease and the elderly are also at increased risk of severe disease. An RSV vaccine containing the AS01 adjuvant, trade name Arexvy, was recently approved in older adults. The antigen is a prefusion-stabilized F glycoprotein (RSVpreF) (SEQ ID NO: 175).

The purpose of this study was to compare the activity of CpG OA-DM2/DOPS to AS01. To this end, BALB/c mice were immunized with 4.8 ug RSVpreF plus 1 ug AS01 of purchased Arexvy. Using the RSVpreF antigen from Arexvy, mice were also immunized with a dose range of CpG OA-DM2/DOPS (3, 1, 0.3 or 0.1 ug) or free CpG (50 or 10 ug). Mice were bled on day 25 post-vaccination and total binding IgG titers were determined by limiting dilution ELISA.

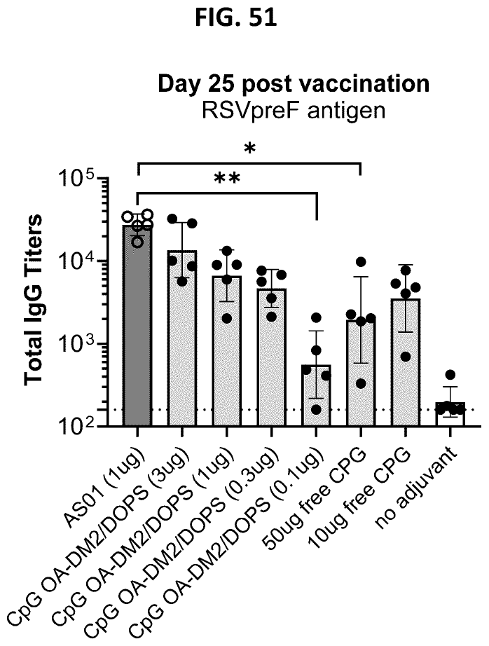

The AS01 adjuvant induced robust levels of anti-RSVpreF antibodies, and titers were comparable at the 3, 1 and 0.3 ug CpG OA-DM2/DOPS doses with a downward trend as the dose decreased. 0.1 ug CpG OA-DM2/DOPS was poorly immunogenic (FIG. 51). Free CpG was less effective at inducing antibodies, although this was not statistically significant. The geometric mean and SD for 3 ug CpG OA-DM2/DOPS was 13560 (2.159), 1946 (3.327) for 50 ug free CpG, and 3546 (2.554) for 10 ug free CpG. In conclusion, the CpG OA-DM2/DOPS adjuvant was able to efficiently boost antibody titers to the RSVpreF antigen.

RSV prefusion F. DS-Cav1 vaccine antigen.
SEQ ID NO: 175
MELLILKANAITTILTAVTFCFASGQNITEEFYQSTCSAVSKGYL
SALRTGWYTSVITIELSNIKENKCNGTDAKVKLIKQELDKYKNAV
TELQLLMQSTPPTNNRARRELPRFMNYTLNNAKKTNVTLSKKRKR
RFLGFLLGVGSAIASGVAVSKVLHLEGEVNKIKSALLSTNKAVVS
LSNGVSVLTSKVLDLKNYIDKQLLPIVNKQSCSISNIETVIEFQQ
KNNRLLEITREFSVNAGVTTPVSTYMLTNSELLSLINDMPITNDQ
KKLMSNNVQIVRQQSYSIMSIIKEEVLAYVVQLPLYGVIDTPCWK
LHTSPLCTTNTKEGSNICLTRTDRGWYCDNAGSVSFFPQAETCKV
QSNR VFCDTMNSLTLPSEINLCNVDIFNPKYDCKIMTSKTDVSS
SVITSLGAIVSCYGKTKCTASNKNRGIIKTFSNGCDYVSNKGMDT
VSVGNTLYYVNKQEGKSLYVKGEPIINFYDPLVFPSDEFDASISQ
VNEKINQSLAFIRKSDELLHNVNAGKSTTNIMITTIIIVIIVILL
SLIAVGLLLYCKARSTPVTLSKDQLSGINNIAFSN.

### Example 37. Potential Uses of Adjuvanted LNPs with Subunit Antigens

Vaccines using recombinant protein antigens often lack the pathogen-associated molecular patterns (PAMPs) or danger-associated molecular patterns (DAMPs) needed to engage with germline-encoded pattern recognition receptors and activate innate immune cells. By co-delivering an adjuvant with the antigen, innate and adaptive immune responses become more robust and provide better immunity and memory T and B cells form longer lasting memory. Adjuvants are also dose sparing—they can reduce the number of vaccinations needed to protect against infection or disease, and less antigen is required. There are few approved adjuvants available, which limits access to these adjuvants. The same short list of adjuvants is used to protect against many types of infectious diseases, and the adjuvant may not be optimal for the type of desired immune response, (e.g. Th1 vs. Th17 CD4 T cell differentiation). The use of TLR7/8 and TLR9 adjuvants, which promote robust Th1-like immune responses, have been limited in their use largely due to unacceptable off-target effects (e.g. TLR7/8 agonists such as Resiquimod and R848) and inefficient delivery (e.g. CpG ODNs).

TLR9 CpG-LNP and TLR7/8-LNP adjuvants could improve immune responses to many vaccine antigens. In one embodiment, hepatitis B vaccine (recombinant) is a sterile suspension of noninfectious HBsAg (SEQ ID NO: 178) for intramuscular administration. It contains purified surface antigen of the virus obtained by culturing genetically engineered Saccharomyces cerevisiae cells, which carry the surface antigen gene of the hepatitis B virus. The HBsAg is formulated as a suspension of the antigen with CpG encapsulated in PS-targeted LNPs or UUGU encapsulated in PS-targeted LNPs.

In another embodiment, hepatitis B vaccine (recombinant) is a sterile suspension of noninfectious HBsAg for intramuscular administration and it is prepared by combining purified HBsAg together with the CpG-LNP adjuvant consisting of a 22-mer phosphorothioate linked oligodeoxynucleotide encapsulated in LNPs in a phosphate buffered saline.

In another embodiment, hepatitis B vaccine (recombinant) is a sterile suspension of noninfectious HBsAg for intramuscular administration and it is prepared by combining purified HBsAg together with UUGU-LNP adjuvant.

In another embodiment, hepatitis B vaccine (recombinant) is a sterile suspension of noninfectious HBsAg consisting of the small (S), middle (pre-S2) and large (preS1) proteins co-purified from genetically modified CHO (Chinese Hamster Ovary) cells cultured in growth medium containing vitamins, amino acids, minerals, and fetal bovine serum. Each dose is formulated to contain 10 mcg hepatitis B surface antigens suspended with CpG-LNP adjuvant or UUGU-LNP adjuvant.

In another embodiment, Zoster Vaccine Recombinant (Adjuvanted) is a sterile injectable suspension for intramuscular use. The vaccine is supplied as a vial of lyophilized recombinant varicella zoster virus surface glycoprotein E (gE) antigen component, which must be reconstituted at the time of use with the accompanying vial of the CpG-LNP or UUGU-LNP adjuvant suspension component. The lyophilized gE antigen component is presented in the form of a sterile white powder.

In another embodiment, R21 malaria vaccine (recombinant, adjuvanted) is a sterile injectable suspension for intramuscular use. The vaccine is supplied as a vial of R21 antigen (SEQ ID NO: 176) consisting of a portion of P. falciparum Circumsporozoite protein fused with hepatitis B surface antigen in the form of non-infectious virus-like particles (VLPs) produced in yeast cells by recombinant DNA technology. The antigen is in suspension with CpG-LNP or UUGU-LNP adjuvant.

In another embodiment, RTS,S malaria vaccine (recombinant, adjuvanted) is a sterile injectable suspension for intramuscular use. The vaccine is supplied as a 25 ug dose of RTS,S antigen (SEQ ID NO: 177) consisting of a portion of P. falciparum Circumsporozoite protein fused with hepatitis B surface antigen in the form of non-infectious virus-like particles (VLPs) produced in yeast cells by recombinant DNA technology. The antigen is in suspension with CpG-LNP or UUGU-LNP adjuvant.

In another embodiment, M72 M. tuberculosis vaccine (recombinant, adjuvanted) is a sterile injectable suspension for intramuscular use. The vaccine consists of the M72 recombinant fusion protein (SEQ ID NO: 179) and CpG-LNP or UUGU-LNP adjuvant.

In another embodiment, respiratory syncytial virus (RSV) vaccine (recombinant, adjuvanted) is a sterile injectable suspension for intramuscular use. The vaccine is supplied as a vial of lyophilized recombinant respiratory syncytial virus glycoprotein F stabilized in pre-fusion conformation (RSVPreF3) as the antigen component (SEQ ID NO: 178), which must be reconstituted at the time of use with the accompanying vial of CpG-LNP or UUGU-LNP adjuvant as the suspension component. The RSVPreF3 antigen is expressed by culturing genetically engineered Chinese Hamster Ovary cells in media containing no antibiotics or animal-derived proteins. The RSVPreF3 protein is purified by several chromatographic and filtration steps, formulated with excipients, filled into vials, and lyophilized.

In another embodiment, RSV vaccine (recombinant, adjuvanted) is a sterile injectable suspension for intramuscular use. The vaccine is supplied as a vial of lyophilized recombinant RSVpreF from the A strain and the B strain as the antigen component, which must be reconstituted at the time of use with the accompanying vial of CpG-LNP or UUGU-LNP adjuvant as the suspension component.

In another embodiment, RSV vaccine (recombinant, adjuvanted) is a sterile injectable suspension for intramuscular use. The vaccine is supplied as a vial of lyophilized recombinant RSVpreF ferritin nanoparticles (SEQ ID NO: 174), which must be reconstituted at the time of use with the accompanying vial of CpG-LNP or UUGU-LNP adjuvant as the suspension component.

In another embodiment, influenza H3 hemagglutinin vaccine (recombinant, adjuvanted) is a sterile injectable suspension for intramuscular use. The vaccine is supplied as a vial of lyophilized recombinant stabilized stem subtype H3 ferritin nanoparticles (SEQ ID NO: 180), which must be reconstituted at the time of use with the accompanying vial of CpG-LNP or UUGU-LNP adjuvant as the suspension component.

In another embodiment, influenza H10 hemagglutinin vaccine (recombinant, adjuvanted) is a sterile injectable suspension for intramuscular use. The vaccine is supplied as a vial of lyophilized recombinant stabilized stem subtype H10 ferritin nanoparticles (SEQ ID NO: 181), which must be reconstituted at the time of use with the accompanying vial of CpG-LNP or UUGU-LNP adjuvant as the suspension component.

In another embodiment, influenza H1 hemagglutinin vaccine (recombinant, adjuvanted) is a sterile injectable suspension for intramuscular use. The vaccine is supplied as a vial of lyophilized recombinant stabilized stem subtype H1 ferritin nanoparticles (SEQ ID NO: 182), which must be reconstituted at the time of use with the accompanying vial of CpG-LNP or UUGU-LNP adjuvant as the suspension component.

In another embodiment, influenza H7 hemagglutinin vaccine (recombinant, adjuvanted) is a sterile injectable suspension for intramuscular use. The vaccine is supplied as a vial of lyophilized recombinant stabilized stem subtype H7 ferritin nanoparticles (SEQ ID NO: 183), which must be reconstituted at the time of use with the accompanying vial of CpG-LNP or UUGU-LNP adjuvant as the suspension component.

In another embodiment, influenza universal hemagglutinin vaccine (recombinant, adjuvanted) is a sterile injectable suspension for intramuscular use. The vaccine is supplied as a vial of lyophilized recombinant stabilized stem subtype H1/H3/H7/H10 ferritin nanoparticles (SEQ ID NO: 180-183), which must be reconstituted at the time of use with the accompanying vial of CpG-LNP or UUGU-LNP adjuvant as the suspension component.

In another embodiment, mumps virus vaccine (recombinant, adjuvanted) is a sterile injectable suspension for intramuscular use. The vaccine is supplied as a vial of lyophilized recombinant stabilized mumps virus F ectodomain trimer antigen (SEQ ID NO: 184), which must be reconstituted at the time of use with the accompanying vial of CpG-LNP or UUGU-LNP adjuvant as the suspension component.

In another embodiment, measles virus vaccine (recombinant, adjuvanted) is a sterile injectable suspension for intramuscular use. The vaccine is supplied as a vial of lyophilized recombinant stabilized measles virus F ectodomain trimer antigen. (SEQ ID NO: 185), which must be reconstituted at the time of use with the accompanying vial of CpG-LNP or UUGU-LNP adjuvant as the suspension component.

In another embodiment, human immunodeficiency virus vaccine (recombinant, adjuvanted) is a sterile injectable suspension for intramuscular use. The vaccine is supplied as a vial of lyophilized recombinant HIV envelop engineered outer domain antigen (eOD-GT8). (SEQ ID NO: 186), which must be reconstituted at the time of use with the accompanying vial of CpG-LNP or UUGU-LNP adjuvant as the suspension component.

In another embodiment, COVID-19 vaccine (adjuvanted) is a sterile injectable suspension for intramuscular use that is free from visible particles. Each 0.5 mL dose of COVID-19 vaccine contains 5 mcg of recombinant spike (rS) protein of the SARS-CoV-2 Omicron variant lineage JN.1 and CpG-LNP or UUGU-LNP adjuvant.

In another embodiment, Chikungunya Vaccine, Recombinant, is a sterile injectable suspension for intramuscular use. The vaccine contains purified virus-like particles (VLPs) in suspension consisting of CHIKV capsid protein (C) and envelope proteins E1 and E2, derived from CHIKV Senegal strain 37997. Each 0.8-mL dose contains approximately 40 mcg of CHIKV VLPs and CpG-LNP or UUGU-LNP adjuvant.

In another embodiment, Hepatitis A & Hepatitis B (Recombinant) vaccine is a bivalent vaccine containing the antigenic components used in producing HAVRIX (Hepatitis A Vaccine) and ENGERIX-B [Hepatitis B Vaccine (Recombinant)]. Hepatitis A & Hepatitis B vaccine is a sterile suspension for intramuscular administration that contains inactivated hepatitis A virus (strain HM175) and noninfectious HBsAg. The hepatitis A virus is propagated in MRC-5 human diploid cells and inactivated with formalin. A 1-mL dose of vaccine contains 720 ELISA Units of inactivated hepatitis A virus and 20 mcg of recombinant HBsAg protein. One dose of vaccine also contains CpG-LNP or UUGU-LNP adjuvant.

In another embodiment, Anthrax Vaccine, Adjuvanted, is a sterile injectable suspension for intramuscular use. The vaccine is prepared from sterile culture filtrates containing proteins released during growth, including the 83-kDa protective antigen protein, and contains no dead or live bacteria. The sterile culture filtrates containing the proteins are in suspension with CpG-LNP or UUGU-LNP adjuvant.

In another embodiment, Human Papillomavirus 9-valent Vaccine, Recombinant, is a non-infectious recombinant 9-valent vaccine prepared from the purified virus-like particles (VLPs) of the major capsid (L1) protein of HPV Types 6 (SEQ ID NOs: 187 and 188), 11 (SEQ ID NO: 189), 16 (SEQ ID NO: 190), 18 (SEQ ID NO: 191), 31 (SEQ ID NO: 192), 33 (SEQ ID NO: 193), 45 (SEQ ID NO: 194), 52 (SEQ ID NO: 195), and 58 (SEQ ID NO: 196). The L1 proteins are produced by separate fermentations using recombinant Saccharomyces cerevisiae and self-assembled into VLPs. The purified VLPs are in suspension with CpG-LNP or UUGU-LNP adjuvant.

In another embodiment, Human Papillomavirus bivalent Vaccine, Recombinant, is a non-infectious recombinant bivalent vaccine prepared from the purified virus-like particles (VLPs) of the major capsid (L1) protein of HPV Types 16 (SEQ ID NO: 190) and 18 (SEQ ID NO: 191). The L1 proteins are produced by separate fermentations using recombinant Saccharomyces cerevisiae and self-assembled into VLPs. The purified VLPs are in suspension with CpG-LNP or UUGU-LNP adjuvant.

In another embodiment, Human Papillomavirus quadrivalent Vaccine, Recombinant, is a non-infectious recombinant bivalent vaccine prepared from the purified virus-like particles (VLPs) of the major capsid (L1) protein of HPV Types 16 (SEQ ID NO: 190), 18 (SEQ ID NO: 191), 6 (SEQ ID NOs: 187 and 188) and 11 (SEQ ID NO: 189). The L1 proteins are produced by separate fermentations using recombinant Saccharomyces cerevisiae and self-assembled into VLPs. The purified VLPs are in suspension with CpG-LNP or UUGU-LNP adjuvant.

Various aspects of the present disclosure may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

While specific embodiments of the subject disclosure have been discussed, the above specification is illustrative and not restrictive. Many variations of the disclosure will become apparent to those skilled in the art upon review of this specification. The full scope of the disclosure should be determined by reference to the claims, along with their full scope of equivalents, and the specification, along with such variations.

All publications, patents and patent applications referenced in this specification are incorporated herein by reference in their entirety for all purposes to the same extent as if each individual publication, patent or patent application were specifically indicated to be so incorporated by reference.

1. A lipid nanoparticle (LNP) composition comprising a TLR-activating nucleic acid.

2. The composition of claim 1, wherein the TLR-activating nucleic acid is a RNA oligonucleotide or a DNA oligonucleotide.

3. The composition of claim 1, wherein the TLR-activating nucleic acid is a TLR9-activating DNA oligonucleotide and one or the nucleic acid is a TLR7/8-activating RNA oligonucleotide.

4. The composition of claim 1, further comprising a TLR4 agonist.

5. The composition of claim 4, wherein the TLR4 agonist is selected from the group consisting of Monophosphoryl Lipid A (MPL), Monophosphoryl Lipid A-504, Monophosphoryl 3-Deacyl Lipid A, and Monophosphoryl Hexa-acyl Lipid A, and 3-Deacyl.

6. The composition of claim 1, wherein the TLR-activating nucleic acid is a TLR9-activating oligonucleotide, optionally CPG1018.

7. The composition of claim 1, wherein the TLR-activating nucleic acid comprises a TLR7 and/or TLR-8 activating RNA oligonucleotide.

8. The composition of claim 1, wherein the TLR-activating nucleic acid comprises both a TLR-activating DNA oligonucleotide and a TLR-activating RNA oligonucleotide.

9. The composition of claim 1, wherein the TLR-activating nucleic acid is a DNA comprising a CpG site.

10. The composition of claim 9, wherein the TLR-activating nucleic acid activates human TLR9.

11. The composition of claim 10, wherein the CpG is selected from the group consisting of: CpG-ODN 1018 (SEQ ID NO. 2), CpG-ODN 2006 (SEQ ID NO. 3; also known as ODN 7909 or PF3512676), CpG-ODN 2216 (SEQ ID NO. 6), CpG-ODN 2336 (SEQ ID NO. 7) and CpG-ODN 2395 (SEQ ID NO. 8)

12. The composition of claim 1, wherein the TLR-activating nucleic acid comprises a TLR7 and/or TLR-8 activating nucleic acid selected from the group consisting of: R-0006 (SEQ ID NO. 25), R-1075 (SEQ ID NO. 11), 9.2dr (SEQ ID NO. 212), 9.3as (SEQ ID NO. 113), 9.2s (SEQ ID NO. 114) and HIV sequence 1 (SEQ ID NO. 116).

13. The composition of claim 1, wherein the TLR-activating nucleic acid activates RIG-I.

14. The composition of claim 13, wherein the TLR-activating nucleic acid is selected from the group consisting of: 3P-GFP2 (SEQ ID NO. 127) annealed with either SEQ ID NO. 128 or SEQ ID NO. 129, 3P-A24 (SEQ ID NO. 130) annealed with either SEQ ID NO. 133 or SEQ ID NO. 134, 3P-G24 (SEQ ID NO. 130) annealed with either SEQ ID NO. 140 or SEQ ID NO. 141, 3P-C24 (SEQ ID NO. 130) annealed with either SEQ ID NO. 147 or SEQ ID NO. 148 and 3P-U24 (SEQ ID NO. 151) annealed with either SEQ ID NO. 154 or SEQ ID NO. 155.

15. A lipid nanoparticle (LNP) composition consisting of:
a. a nucleic acid antigen recognized by a germline-encoded pattern recognition receptor (PRR);
b. an ionizable cationic lipid comprising an ionizable cationic lipid at a N/P ratio of 4 to 6 relative to the mRNA, the ionizable cationic lipid present in the LNP composition in a total amount of 46-54 mol % of a total lipid content of the LNP composition;
c. one or more phospholipids selected from the group consisting of distearoylphosphatidylcholine (DSPC), hydrogenated soy phosphatidylcholine (HSPC), and dipalmitoylphosphatidylcholine (DPPC), in a total amount of 10-18 mol % of the total lipid content of the LNP composition;
d. one or more anionic phospholipids selected from the group consisting of dipalmitoylphosphatidyl-L-serine (DPPS), or distearoylphosphatidyl-L-serine (DSPS), distearoylphosphatidylglycerol (DSPG), and dipalmitoyphosphatidylglycerol (DPPG) in a total amount of 2-8 mol % of the total lipid content of the LNP composition;
e. PEG(2000)-dimyristoylglycerol (PEG-DMG) in a total amount of 1-3.5 mol % of the total lipid content of the LNP composition; and
f. cholesterol.

16. The composition of claim 15, wherein the PRR recognizes a conserved microbial pathogen-associated molecular patterns (PAMPs) and danger-associated molecular patterns (DAMPs).

17. The composition of claim 16, wherein:
the nucleic acid antigen is a CpG oligodeoxynucleotide recognized by TLR9; or
the nucleic acid antigen is a ssRNA oligonucleotide recognized by TLR7 or TLR8; or
the nucleic acid antigen is a ssRNA oligonucleotide recognized by TLR7 and TLR8; or
the nucleic acid antigen is a RIG-I that recognizes uncapped 5′ triphosphate RNA (aka pppRNA, 3pRNA); or
the nucleic acid antigen is a 5′ppp ssRNA with polyuridine with interspersed guanosines (poly-U/UG), polyuridine with interspersed cytidines (poly-U/UC), or polyadenosine interspersed with guanosines (poly-A/AG) or with RNA regions enriched with polyuridine and interspersed guanosines (poly-U/UG), polyuridine with interspersed cytidines (poly-U/UC), polyadenosine interspersed with guanosines (poly-A/AG); or
the nucleic acid antigen encodes melanoma differentiation-associated protein 5 (MDA5); or
the nucleic acid antigen is selected from the group consisting of NOD2, LGP2, DDX1-DDX21-DHX36, DDX60, DHX9, DHX36, DDX41, AIM2, IF16, ZBP1, LRRFIP1, and STING; or
the nucleic acid antigen is a CpG oligodeoxynucleotide (ODN) 1018 (SEQ ID NO: 2); or
the nucleic acid antigen is a CpG-ODN 2006 (SEQ ID NO: 3); or
the nucleic acid antigen is TLR7/8 ssRNA oligonucleotide 5′-UUGUUGUUGUUGUUGUUGUU-3′ (SEQ ID NO: 25); or
the nucleic acid antigen is mRNA encoding for SARS-CoV-2 Spike protein (SEQ ID NO: 1); or
the nucleic acid antigen is selected from the group consisting of SEQ ID NO:1, SEQ ID NO:2, or SEQ ID NO:3; or
the nucleic acid antigen is a TLR-9 CpG-ODN adjuvant; or
the nucleic acid antigen is selected from the group consisting of SEQ ID NO:11-SEQ ID NO:117 (Table 55).

18. The composition of claim 2, wherein the nucleic acid is selected from the group consisting of SEQ ID NO:118-SEQ ID NO:126 (Table 56) or SEQ ID NO:127-167 (Table 60).

19. A pharmaceutical composition comprising the lipid nanoparticle composition of claim 1.

20. The pharmaceutical composition of claim 19, wherein the composition is a vaccine.
